SEQUENCE LISTING (1432 sequences)

SEQ ID NO: 1 - protein, 274 aa, SARS-CoV Urbani
  MISC_FEATURE (1)..(274): Amino acid sequence of protein X1
MDLFMRFFTLGSITAQPVKIDNASPASTVHATATIPLQASLPFGWLVIGVAFLAVFQSATKIIALNKRWQLALYKGFQFICNLLLLFVTIYSHLLLVAAGMEAQFLYLYALIYFLQCINACRIIMRCWLCWKCKSKNPLLYDANYFVCWHTHNYDYCIPYNSVTDTIVVTEGDGISTPKLKEDYQIGGYSEDRHSGVKDYVVVHGYFTEVYYQLESTQITTDTGIENATFFIFNKLVKDPPNVQIHTIDGSSGVANPAMDPIYDEPTTTTSVPL

SEQ ID NO: 2 - protein, 154 aa, SARS-CoV Urbani
  MISC_FEATURE (1)..(154): Amino acid sequence of protein X2
MMPTTLFAGTHITMTTVYHITVSQIQLSLLKVTAFQHQNSKKTTKLVVILRIGTQVLKTMSLYMAISPKFTTSLSLHKLLQTLVLKMLHSSSLTSLLKTHRMCKYTQSTALQELLIQQWIQFMMSRRRLLACLCKHKKVSTNLCTHSFRKKQVR

SEQ ID NO: 3 - protein, 76 aa, SARS-CoV Urbani
  MISC_FEATURE (1)..(76): Amino acid sequence of E protein
MYSFVSEETGTLIVNSVLLFLAFVVFLLVTLAILTALRLCAYCCNIVNVSLVKPTVYVYSRVKNLNSSEGVPDLLV

SEQ ID NO: 4 - protein, 221 aa, SARS-CoV Urbani
  MISC_FEATURE (1)..(221): Amino acid sequence of M protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQ

SEQ ID NO: 5 - protein, 63 aa, SARS-CoV Urbani
  MISC_FEATURE (1)..(63): Amino acid sequence of protein X3
MFHLVDFQVTIAEILIIIMRTFRIAIWNLDVIISSIVRQLFKPLTKKNYSELDDEEPMELDYP

SEQ ID NO: 6 - protein, 122 aa, SARS-CoV Urbani
  MISC_FEATURE (1)..(122): Amino acid sequence of protein X4
MKIILFLTLIVFTSCELYHYQECVRGTTVLLKEPCPSGTYEGNSPFHPLADNKFALTCTSTHFAFACADGTRHTYQLRARSVSPKLFIRQEEVQQELYSPLFLIVAALVFLILCFTIKRKTE

SEQ ID NO: 7 - protein, 84 aa, SARS-CoV Urbani
  MISC_FEATURE (1)..(84): Amino acid sequence of protein X5
MCLKILVRYNTRGNTYSTAWLCALGKVLPFHRWHTMVQTCTPNVTINCQDPAGGALIARCWYLHEGHQTAAFRDVLVVLNKRTN

SEQ ID NO: 8 - protein, 422 aa, SARS-CoV Urbani
  MISC_FEATURE (1)..(422): Amino acid sequence of N protein
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 9 - protein, 15 aa, Artificial sequence
  Peptide
RFFTLGSITAQPVKI

SEQ ID NO: 10 - protein, 15 aa, Artificial sequence
  Peptide
FFTLGSITAQPVKID

SEQ ID NO: 11 - protein, 15 aa, Artificial sequence
  Peptide
FTLGSITAQPVKIDN

SEQ ID NO: 12 - protein, 15 aa, Artificial sequence
  Peptide
TLGSITAQPVKIDNA

SEQ ID NO: 13 - protein, 15 aa, Artificial sequence
  Peptide
LGSITAQPVKIDNAS

SEQ ID NO: 14 - protein, 15 aa, Artificial sequence
  Peptide
GSITAQPVKIDNASP

SEQ ID NO: 15 - protein, 15 aa, Artificial sequence
  Peptide
SITAQPVKIDNASPA

SEQ ID NO: 16 - protein, 15 aa, Artificial sequence
  Peptide
ITAQPVKIDNASPAS

SEQ ID NO: 17 - protein, 15 aa, Artificial sequence
  Peptide
TAQPVKIDNASPAST

SEQ ID NO: 18 - protein, 15 aa, Artificial sequence
  Peptide
AQPVKIDNASPASTV

SEQ ID NO: 19 - protein, 15 aa, Artificial sequence
  Peptide
QPVKIDNASPASTVH

SEQ ID NO: 20 - protein, 15 aa, Artificial sequence
  Peptide
PVKIDNASPASTVHA

SEQ ID NO: 21 - protein, 15 aa, Artificial sequence
  Peptide
VKIDNASPASTVHAT

SEQ ID NO: 22 - protein, 15 aa, Artificial sequence
  Peptide
KIDNASPASTVHATA

SEQ ID NO: 23 - protein, 15 aa, Artificial sequence
  Peptide
IDNASPASTVHATAT

SEQ ID NO: 24 - protein, 15 aa, Artificial sequence
  Peptide
DNASPASTVHATATI

SEQ ID NO: 25 - protein, 15 aa, Artificial sequence
  Peptide
NASPASTVHATATIP

SEQ ID NO: 26 - protein, 15 aa, Artificial sequence
  Peptide
ASPASTVHATATIPL

SEQ ID NO: 27 - protein, 15 aa, Artificial sequence
  Peptide
SPASTVHATATIPLQ

SEQ ID NO: 28 - protein, 15 aa, Artificial sequence
  Peptide
PASTVHATATIPLQA

SEQ ID NO: 29 - protein, 15 aa, Artificial sequence
  Peptide
ASTVHATATIPLQAS

SEQ ID NO: 30 - protein, 15 aa, Artificial sequence
  Peptide
STVHATATIPLQASL

SEQ ID NO: 31 - protein, 15 aa, Artificial sequence
  Peptide
TVHATATIPLQASLP

SEQ ID NO: 32 - protein, 15 aa, Artificial sequence
  Peptide
VHATATIPLQASLPF

SEQ ID NO: 33 - protein, 15 aa, Artificial sequence
  Peptide
INACRIIMRCWLCWK

SEQ ID NO: 34 - protein, 15 aa, Artificial sequence
  Peptide
NACRIIMRCWLCWKC

SEQ ID NO: 35 - protein, 15 aa, Artificial sequence
  Peptide
ACRIIMRCWLCWKCK

SEQ ID NO: 36 - protein, 15 aa, Artificial sequence
  Peptide
CRIIMRCWLCWKCKS

SEQ ID NO: 37 - protein, 15 aa, Artificial sequence
  Peptide
RIIMRCWLCWKCKSK

SEQ ID NO: 38 - protein, 15 aa, Artificial sequence
  Peptide
IIMRCWLCWKCKSKN

SEQ ID NO: 39 - protein, 15 aa, Artificial sequence
  Peptide
IMRCWLCWKCKSKNP

SEQ ID NO: 40 - protein, 15 aa, Artificial sequence
  Peptide
MRCWLCWKCKSKNPL

SEQ ID NO: 41 - protein, 15 aa, Artificial sequence
  Peptide
RCWLCWKCKSKNPLL

SEQ ID NO: 42 - protein, 15 aa, Artificial sequence
  Peptide
CWLCWKCKSKNPLLY

SEQ ID NO: 43 - protein, 15 aa, Artificial sequence
  Peptide
WLCWKCKSKNPLLYD

SEQ ID NO: 44 - protein, 15 aa, Artificial sequence
  Peptide
LCWKCKSKNPLLYDA

SEQ ID NO: 45 - protein, 15 aa, Artificial sequence
  Peptide
CWKCKSKNPLLYDAN

SEQ ID NO: 46 - protein, 15 aa, Artificial sequence
  Peptide
YDANYFVCWHTHNYD

SEQ ID NO: 47 - protein, 15 aa, Artificial sequence
  Peptide
DANYFVCWHTHNYDY

SEQ ID NO: 48 - protein, 15 aa, Artificial sequence
  Peptide
ANYFVCWHTHNYDYC

SEQ ID NO: 49 - protein, 15 aa, Artificial sequence
  Peptide
NYFVCWHTHNYDYCI

SEQ ID NO: 50 - protein, 15 aa, Artificial sequence
  Peptide
YFVCWHTHNYDYCIP

SEQ ID NO: 51 - protein, 15 aa, Artificial sequence
  Peptide
FVCWHTHNYDYCIPY

SEQ ID NO: 52 - protein, 15 aa, Artificial sequence
  Peptide
VCWHTHNYDYCIPYN

SEQ ID NO: 53 - protein, 15 aa, Artificial sequence
  Peptide
CWHTHNYDYCIPYNS

SEQ ID NO: 54 - protein, 15 aa, Artificial sequence
  Peptide
WHTHNYDYCIPYNSV

SEQ ID NO: 55 - protein, 15 aa, Artificial sequence
  Peptide
HTHNYDYCIPYNSVT

SEQ ID NO: 56 - protein, 15 aa, Artificial sequence
  Peptide
THNYDYCIPYNSVTD

SEQ ID NO: 57 - protein, 15 aa, Artificial sequence
  Peptide
HNYDYCIPYNSVTDT

SEQ ID NO: 58 - protein, 15 aa, Artificial sequence
  Peptide
NYDYCIPYNSVTDTI

SEQ ID NO: 59 - protein, 15 aa, Artificial sequence
  Peptide
YDYCIPYNSVTDTIV

SEQ ID NO: 60 - protein, 15 aa, Artificial sequence
  Peptide
DYCIPYNSVTDTIVV

SEQ ID NO: 61 - protein, 15 aa, Artificial sequence
  Peptide
YCIPYNSVTDTIVVT

SEQ ID NO: 62 - protein, 15 aa, Artificial sequence
  Peptide
GDGISTPKLKEDYQI

SEQ ID NO: 63 - protein, 15 aa, Artificial sequence
  Peptide
DGISTPKLKEDYQIG

SEQ ID NO: 64 - protein, 15 aa, Artificial sequence
  Peptide
GISTPKLKEDYQIGG

SEQ ID NO: 65 - protein, 15 aa, Artificial sequence
  Peptide
ISTPKLKEDYQIGGY

SEQ ID NO: 66 - protein, 15 aa, Artificial sequence
  Peptide
STPKLKEDYQIGGYS

SEQ ID NO: 67 - protein, 15 aa, Artificial sequence
  Peptide
TPKLKEDYQIGGYSE

SEQ ID NO: 68 - protein, 15 aa, Artificial sequence
  Peptide
PKLKEDYQIGGYSED

SEQ ID NO: 69 - protein, 15 aa, Artificial sequence
  Peptide
KLKEDYQIGGYSEDR

SEQ ID NO: 70 - protein, 15 aa, Artificial sequence
  Peptide
LKEDYQIGGYSEDRH

SEQ ID NO: 71 - protein, 15 aa, Artificial sequence
  Peptide
KEDYQIGGYSEDRHS

SEQ ID NO: 72 - protein, 15 aa, Artificial sequence
  Peptide
EDYQIGGYSEDRHSG

SEQ ID NO: 73 - protein, 15 aa, Artificial sequence
  Peptide
DYQIGGYSEDRHSGV

SEQ ID NO: 74 - protein, 15 aa, Artificial sequence
  Peptide
YQIGGYSEDRHSGVK

SEQ ID NO: 75 - protein, 15 aa, Artificial sequence
  Peptide
QIGGYSEDRHSGVKD

SEQ ID NO: 76 - protein, 15 aa, Artificial sequence
  Peptide
IGGYSEDRHSGVKDY

SEQ ID NO: 77 - protein, 15 aa, Artificial sequence
  Peptide
GGYSEDRHSGVKDYV

SEQ ID NO: 78 - protein, 15 aa, Artificial sequence
  Peptide
GYSEDRHSGVKDYVV

SEQ ID NO: 79 - protein, 15 aa, Artificial sequence
  Peptide
YSEDRHSGVKDYVVV

SEQ ID NO: 80 - protein, 15 aa, Artificial sequence
  Peptide
SEDRHSGVKDYVVVH

SEQ ID NO: 81 - protein, 15 aa, Artificial sequence
  Peptide
EDRHSGVKDYVVVHG

SEQ ID NO: 82 - protein, 15 aa, Artificial sequence
  Peptide
DRHSGVKDYVVVHGY

SEQ ID NO: 83 - protein, 15 aa, Artificial sequence
  Peptide
RHSGVKDYVVVHGYF

SEQ ID NO: 84 - protein, 15 aa, Artificial sequence
  Peptide
HSGVKDYVVVHGYFT

SEQ ID NO: 85 - protein, 15 aa, Artificial sequence
  Peptide
SGVKDYVVVHGYFTE

SEQ ID NO: 86 - protein, 15 aa, Artificial sequence
  Peptide
GVKDYVVVHGYFTEV

SEQ ID NO: 87 - protein, 15 aa, Artificial sequence
  Peptide
ATFFIFNKLVKDPPN

SEQ ID NO: 88 - protein, 15 aa, Artificial sequence
  Peptide
TFFIFNKLVKDPPNV

SEQ ID NO: 89 - protein, 15 aa, Artificial sequence
  Peptide
FFIFNKLVKDPPNVQ

SEQ ID NO: 90 - protein, 15 aa, Artificial sequence
  Peptide
FIFNKLVKDPPNVQI

SEQ ID NO: 91 - protein, 15 aa, Artificial sequence
  Peptide
IFNKLVKDPPNVQIH

SEQ ID NO: 92 - protein, 15 aa, Artificial sequence
  Peptide
FNKLVKDPPNVQIHT

SEQ ID NO: 93 - protein, 15 aa, Artificial sequence
  Peptide
NKLVKDPPNVQIHTI

SEQ ID NO: 94 - protein, 15 aa, Artificial sequence
  Peptide
KLVKDPPNVQIHTID

SEQ ID NO: 95 - protein, 15 aa, Artificial sequence
  Peptide
LVKDPPNVQIHTIDG

SEQ ID NO: 96 - protein, 15 aa, Artificial sequence
  Peptide
VKDPPNVQIHTIDGS

SEQ ID NO: 97 - protein, 15 aa, Artificial sequence
  Peptide
KDPPNVQIHTIDGSS

SEQ ID NO: 98 - protein, 15 aa, Artificial sequence
  Peptide
DGSSGVANPAMDPIY

SEQ ID NO: 99 - protein, 15 aa, Artificial sequence
  Peptide
GSSGVANPAMDPIYD

SEQ ID NO: 100 - protein, 15 aa, Artificial sequence
  Peptide
SSGVANPAMDPIYDE

SEQ ID NO: 101 - protein, 15 aa, Artificial sequence
  Peptide
SGVANPAMDPIYDEP

SEQ ID NO: 102 - protein, 15 aa, Artificial sequence
  Peptide
GVANPAMDPIYDEPT

SEQ ID NO: 103 - protein, 15 aa, Artificial sequence
  Peptide
VANPAMDPIYDEPTT

SEQ ID NO: 104 - protein, 15 aa, Artificial sequence
  Peptide
ANPAMDPIYDEPTTT

SEQ ID NO: 105 - protein, 15 aa, Artificial sequence
  Peptide
NPAMDPIYDEPTTTT

SEQ ID NO: 106 - protein, 15 aa, Artificial sequence
  Peptide
PAMDPIYDEPTTTTS

SEQ ID NO: 107 - protein, 15 aa, Artificial sequence
  Peptide
AMDPIYDEPTTTTSV

SEQ ID NO: 108 - protein, 15 aa, Artificial sequence
  Peptide
MDPIYDEPTTTTSVP

SEQ ID NO: 109 - protein, 15 aa, Artificial sequence
  Peptide
DPIYDEPTTTTSVPL

SEQ ID NO: 110 - protein, 15 aa, Artificial sequence
  Peptide
MMPTTLFAGTHITMT

SEQ ID NO: 111 - protein, 15 aa, Artificial sequence
  Peptide
MPTTLFAGTHITMTT

SEQ ID NO: 112 - protein, 15 aa, Artificial sequence
  Peptide
PTTLFAGTHITMTTV

SEQ ID NO: 113 - protein, 15 aa, Artificial sequence
  Peptide
TTLFAGTHITMTTVY

SEQ ID NO: 114 - protein, 15 aa, Artificial sequence
  Peptide
TLFAGTHITMTTVYH

SEQ ID NO: 115 - protein, 15 aa, Artificial sequence
  Peptide
LFAGTHITMTTVYHI

SEQ ID NO: 116 - protein, 15 aa, Artificial sequence
  Peptide
FAGTHITMTTVYHIT

SEQ ID NO: 117 - protein, 15 aa, Artificial sequence
  Peptide
AGTHITMTTVYHITV

SEQ ID NO: 118 - protein, 15 aa, Artificial sequence
  Peptide
GTHITMTTVYHITVS

SEQ ID NO: 119 - protein, 15 aa, Artificial sequence
  Peptide
FQHQNSKKTTKLVVI

SEQ ID NO: 120 - protein, 15 aa, Artificial sequence
  Peptide
QHQNSKKTTKLVVIL

SEQ ID NO: 121 - protein, 15 aa, Artificial sequence
  Peptide
HQNSKKTTKLVVILR

SEQ ID NO: 122 - protein, 15 aa, Artificial sequence
  Peptide
QNSKKTTKLVVILRI

SEQ ID NO: 123 - protein, 15 aa, Artificial sequence
  Peptide
NSKKTTKLVVILRIG

SEQ ID NO: 124 - protein, 15 aa, Artificial sequence
  Peptide
SKKTTKLVVILRIGT

SEQ ID NO: 125 - protein, 15 aa, Artificial sequence
  Peptide
KKTTKLVVILRIGTQ

SEQ ID NO: 126 - protein, 15 aa, Artificial sequence
  Peptide
KTTKLVVILRIGTQV

SEQ ID NO: 127 - protein, 15 aa, Artificial sequence
  Peptide
TTKLVVILRIGTQVL

SEQ ID NO: 128 - protein, 15 aa, Artificial sequence
  Peptide
TKLVVILRIGTQVLK

SEQ ID NO: 129 - protein, 15 aa, Artificial sequence
  Peptide
KLVVILRIGTQVLKT

SEQ ID NO: 130 - protein, 15 aa, Artificial sequence
  Peptide
LRIGTQVLKTMSLYM

SEQ ID NO: 131 - protein, 15 aa, Artificial sequence
  Peptide
RIGTQVLKTMSLYMA

SEQ ID NO: 132 - protein, 15 aa, Artificial sequence
  Peptide
IGTQVLKTMSLYMAI

SEQ ID NO: 133 - protein, 15 aa, Artificial sequence
  Peptide
GTQVLKTMSLYMAIS

SEQ ID NO: 134 - protein, 15 aa, Artificial sequence
  Peptide
TQVLKTMSLYMAISP

SEQ ID NO: 135 - protein, 15 aa, Artificial sequence
  Peptide
QVLKTMSLYMAISPK

SEQ ID NO: 136 - protein, 15 aa, Artificial sequence
  Peptide
VLKTMSLYMAISPKF

SEQ ID NO: 137 - protein, 15 aa, Artificial sequence
  Peptide
LKTMSLYMAISPKFT

SEQ ID NO: 138 - protein, 15 aa, Artificial sequence
  Peptide
KTMSLYMAISPKFTT

SEQ ID NO: 139 - protein, 15 aa, Artificial sequence
  Peptide
MMSRRRLLACLCKHK

SEQ ID NO: 140 - protein, 15 aa, Artificial sequence
  Peptide
MSRRRLLACLCKHKK

SEQ ID NO: 141 - protein, 15 aa, Artificial sequence
  Peptide
SRRRLLACLCKHKKV

SEQ ID NO: 142 - protein, 15 aa, Artificial sequence
  Peptide
RRRLLACLCKHKKVS

SEQ ID NO: 143 - protein, 15 aa, Artificial sequence
  Peptide
RRLLACLCKHKKVST

SEQ ID NO: 144 - protein, 15 aa, Artificial sequence
  Peptide
RLLACLCKHKKVSTN

SEQ ID NO: 145 - protein, 15 aa, Artificial sequence
  Peptide
LLACLCKHKKVSTNL

SEQ ID NO: 146 - protein, 15 aa, Artificial sequence
  Peptide
LACLCKHKKVSTNLC

SEQ ID NO: 147 - protein, 15 aa, Artificial sequence
  Peptide
ACLCKHKKVSTNLCT

SEQ ID NO: 148 - protein, 15 aa, Artificial sequence
  Peptide
CLCKHKKVSTNLCTH

SEQ ID NO: 149 - protein, 15 aa, Artificial sequence
  Peptide
LCKHKKVSTNLCTHS

SEQ ID NO: 150 - protein, 15 aa, Artificial sequence
  Peptide
CKHKKVSTNLCTHSF

SEQ ID NO: 151 - protein, 15 aa, Artificial sequence
  Peptide
KHKKVSTNLCTHSFR

SEQ ID NO: 152 - protein, 15 aa, Artificial sequence
  Peptide
HKKVSTNLCTHSFRK

SEQ ID NO: 153 - protein, 15 aa, Artificial sequence
  Peptide
KKVSTNLCTHSFRKK

SEQ ID NO: 154 - protein, 15 aa, Artificial sequence
  Peptide
KVSTNLCTHSFRKKQ

SEQ ID NO: 155 - protein, 15 aa, Artificial sequence
  Peptide
VSTNLCTHSFRKKQV

SEQ ID NO: 156 - protein, 15 aa, Artificial sequence
  Peptide
STNLCTHSFRKKQVR

SEQ ID NO: 157 - protein, 15 aa, Artificial sequence
  Peptide
LCAYCCNIVNVSLVK

SEQ ID NO: 158 - protein, 15 aa, Artificial sequence
  Peptide
CAYCCNIVNVSLVKP

SEQ ID NO: 159 - protein, 15 aa, Artificial sequence
  Peptide
AYCCNIVNVSLVKPT

SEQ ID NO: 160 - protein, 15 aa, Artificial sequence
  Peptide
YCCNIVNVSLVKPTV

SEQ ID NO: 161 - protein, 15 aa, Artificial sequence
  Peptide
CCNIVNVSLVKPTVY

SEQ ID NO: 162 - protein, 15 aa, Artificial sequence
  Peptide
CNIVNVSLVKPTVYV

SEQ ID NO: 163 - protein, 15 aa, Artificial sequence
  Peptide
NIVNVSLVKPTVYVY

SEQ ID NO: 164 - protein, 15 aa, Artificial sequence
  Peptide
IVNVSLVKPTVYVYS

SEQ ID NO: 165 - protein, 15 aa, Artificial sequence
  Peptide
VNVSLVKPTVYVYSR

SEQ ID NO: 166 - protein, 15 aa, Artificial sequence
  Peptide
NVSLVKPTVYVYSRV

SEQ ID NO: 167 - protein, 15 aa, Artificial sequence
  Peptide
VSLVKPTVYVYSRVK

SEQ ID NO: 168 - protein, 15 aa, Artificial sequence
  Peptide
SLVKPTVYVYSRVKN

SEQ ID NO: 169 - protein, 15 aa, Artificial sequence
  Peptide
LVKPTVYVYSRVKNL

SEQ ID NO: 170 - protein, 15 aa, Artificial sequence
  Peptide
VKPTVYVYSRVKNLN

SEQ ID NO: 171 - protein, 15 aa, Artificial sequence
  Peptide
KPTVYVYSRVKNLNS

SEQ ID NO: 172 - protein, 15 aa, Artificial sequence
  Peptide
PTVYVYSRVKNLNSS

SEQ ID NO: 173 - protein, 15 aa, Artificial sequence
  Peptide
TVYVYSRVKNLNSSE

SEQ ID NO: 174 - protein, 15 aa, Artificial sequence
  Peptide
VYVYSRVKNLNSSEG

SEQ ID NO: 175 - protein, 15 aa, Artificial sequence
  Peptide
YVYSRVKNLNSSEGV

SEQ ID NO: 176 - protein, 15 aa, Artificial sequence
  Peptide
VYSRVKNLNSSEGVP

SEQ ID NO: 177 - protein, 15 aa, Artificial sequence
  Peptide
YSRVKNLNSSEGVPD

SEQ ID NO: 178 - protein, 15 aa, Artificial sequence
  Peptide
SRVKNLNSSEGVPDL

SEQ ID NO: 179 - protein, 15 aa, Artificial sequence
  Peptide
RVKNLNSSEGVPDLL

SEQ ID NO: 180 - protein, 15 aa, Artificial sequence
  Peptide
VKNLNSSEGVPDLLV

SEQ ID NO: 181 - protein, 15 aa, Artificial sequence
  Peptide
MADNGTITVEELKQL

SEQ ID NO: 182 - protein, 15 aa, Artificial sequence
  Peptide
ADNGTITVEELKQLL

SEQ ID NO: 183 - protein, 15 aa, Artificial sequence
  Peptide
DNGTITVEELKQLLE

SEQ ID NO: 184 - protein, 15 aa, Artificial sequence
  Peptide
NGTITVEELKQLLEQ

SEQ ID NO: 185 - protein, 15 aa, Artificial sequence
  Peptide
GTITVEELKQLLEQW

SEQ ID NO: 186 - protein, 15 aa, Artificial sequence
  Peptide
TITVEELKQLLEQWN

SEQ ID NO: 187 - protein, 15 aa, Artificial sequence
  Peptide
ITVEELKQLLEQWNL

SEQ ID NO: 188 - protein, 15 aa, Artificial sequence
  Peptide
TVEELKQLLEQWNLV

SEQ ID NO: 189 - protein, 15 aa, Artificial sequence
  Peptide
VEELKQLLEQWNLVI

SEQ ID NO: 190 - protein, 15 aa, Artificial sequence
  Peptide
EELKQLLEQWNLVIG

SEQ ID NO: 191 - protein, 15 aa, Artificial sequence
  Peptide
QFAYSNRNRFLYIIK

SEQ ID NO: 192 - protein, 15 aa, Artificial sequence
  Peptide
FAYSNRNRFLYIIKL

SEQ ID NO: 193 - protein, 15 aa, Artificial sequence
  Peptide
AYSNRNRFLYIIKLV

SEQ ID NO: 194 - protein, 15 aa, Artificial sequence
  Peptide
YSNRNRFLYIIKLVF

SEQ ID NO: 195 - protein, 15 aa, Artificial sequence
  Peptide
SNRNRFLYIIKLVFL

SEQ ID NO: 196 - protein, 15 aa, Artificial sequence
  Peptide
NRNRFLYIIKLVFLW

SEQ ID NO: 197 - protein, 15 aa, Artificial sequence
  Peptide
RNRFLYIIKLVFLWL

SEQ ID NO: 198 - protein, 15 aa, Artificial sequence
  Peptide
NRFLYIIKLVFLWLL

SEQ ID NO: 199 - protein, 15 aa, Artificial sequence
  Peptide
RFLYIIKLVFLWLLW

SEQ ID NO: 200 - protein, 15 aa, Artificial sequence
  Peptide
FLYIIKLVFLWLLWP

SEQ ID NO: 201 - protein, 15 aa, Artificial sequence
  Peptide
INWVTGGIAIAMACI

SEQ ID NO: 202 - protein, 15 aa, Artificial sequence
  Peptide
NWVTGGIAIAMACIV

SEQ ID NO: 203 - protein, 15 aa, Artificial sequence
  Peptide
WVTGGIAIAMACIVG

SEQ ID NO: 204 - protein, 15 aa, Artificial sequence
  Peptide
VTGGIAIAMACIVGL

SEQ ID NO: 205 - protein, 15 aa, Artificial sequence
  Peptide
TGGIAIAMACIVGLM

SEQ ID NO: 206 - protein, 15 aa, Artificial sequence
  Peptide
GGIAIAMACIVGLMW

SEQ ID NO: 207 - protein, 15 aa, Artificial sequence
  Peptide
GIAIAMACIVGLMWL

SEQ ID NO: 208 - protein, 15 aa, Artificial sequence
  Peptide
IAIAMACIVGLMWLS

SEQ ID NO: 209 - protein, 15 aa, Artificial sequence
  Peptide
LMWLSYFVASFRLFA

SEQ ID NO: 210 - protein, 15 aa, Artificial sequence
  Peptide
MWLSYFVASFRLFAR

SEQ ID NO: 211 - protein, 15 aa, Artificial sequence
  Peptide
WLSYFVASFRLFART

SEQ ID NO: 212 - protein, 15 aa, Artificial sequence
  Peptide
LSYFVASFRLFARTR

SEQ ID NO: 213 - protein, 15 aa, Artificial sequence
  Peptide
SYFVASFRLFARTRS

SEQ ID NO: 214 - protein, 15 aa, Artificial sequence
  Peptide
YFVASFRLFARTRSM

SEQ ID NO: 215 - protein, 15 aa, Artificial sequence
  Peptide
FVASFRLFARTRSMW

SEQ ID NO: 216 - protein, 15 aa, Artificial sequence
  Peptide
VASFRLFARTRSMWS

SEQ ID NO: 217 - protein, 15 aa, Artificial sequence
  Peptide
NILLNVPLRGTIVTR

SEQ ID NO: 218 - protein, 15 aa, Artificial sequence
  Peptide
ILLNVPLRGTIVTRP

SEQ ID NO: 219 - protein, 15 aa, Artificial sequence
  Peptide
LLNVPLRGTIVTRPL

SEQ ID NO: 220 - protein, 15 aa, Artificial sequence
  Peptide
LNVPLRGTIVTRPLM

SEQ ID NO: 221 - protein, 15 aa, Artificial sequence
  Peptide
NVPLRGTIVTRPLME

SEQ ID NO: 222 - protein, 15 aa, Artificial sequence
  Peptide
VPLRGTIVTRPLMES

SEQ ID NO: 223 - protein, 15 aa, Artificial sequence
  Peptide
PLRGTIVTRPLMESE

SEQ ID NO: 224 - protein, 15 aa, Artificial sequence
  Peptide
LRGTIVTRPLMESEL

SEQ ID NO: 225 - protein, 15 aa, Artificial sequence
  Peptide
RGTIVTRPLMESELV

SEQ ID NO: 226 - protein, 15 aa, Artificial sequence
  Peptide
GTIVTRPLMESELVI

SEQ ID NO: 227 - protein, 15 aa, Artificial sequence
  Peptide
TIVTRPLMESELVIG

SEQ ID NO: 228 - skipped

SEQ ID NO: 229 - protein, 15 aa, Artificial sequence
  Peptide
IVTRPLMESELVIGA

SEQ ID NO: 230 - protein, 15 aa, Artificial sequence
  Peptide
VTRPLMESELVIGAV

SEQ ID NO: 231 - protein, 15 aa, Artificial sequence
  Peptide
TRPLMESELVIGAVI

SEQ ID NO: 232 - protein, 15 aa, Artificial sequence
  Peptide
RPLMESELVIGAVII

SEQ ID NO: 233 - protein, 15 aa, Artificial sequence
  Peptide
VIGAVIIRGHLRMAG

SEQ ID NO: 234 - protein, 15 aa, Artificial sequence
  Peptide
IGAVIIRGHLRMAGH

SEQ ID NO: 235 - protein, 15 aa, Artificial sequence
  Peptide
GAVIIRGHLRMAGHP

SEQ ID NO: 236 - protein, 15 aa, Artificial sequence
  Peptide
AVIIRGHLRMAGHPL

SEQ ID NO: 237 - protein, 15 aa, Artificial sequence
  Peptide
VIIRGHLRMAGHPLG

SEQ ID NO: 238 - protein, 15 aa, Artificial sequence
  Peptide
IIRGHLRMAGHPLGR

SEQ ID NO: 239 - protein, 15 aa, Artificial sequence
  Peptide
IRGHLRMAGHPLGRC

SEQ ID NO: 240 - protein, 15 aa, Artificial sequence
  Peptide
RGHLRMAGHPLGRCD

SEQ ID NO: 241 - protein, 15 aa, Artificial sequence
  Peptide
GHLRMAGHPLGRCDI

SEQ ID NO: 242 - protein, 15 aa, Artificial sequence
  Peptide
HLRMAGHPLGRCDIK

SEQ ID NO: 243 - protein, 15 aa, Artificial sequence
  Peptide
LRMAGHPLGRCDIKD

SEQ ID NO: 244 - protein, 15 aa, Artificial sequence
  Peptide
RMAGHPLGRCDIKDL

SEQ ID NO: 245 - protein, 15 aa, Artificial sequence
  Peptide
MAGHPLGRCDIKDLP

SEQ ID NO: 246 - protein, 15 aa, Artificial sequence
  Peptide
AGHPLGRCDIKDLPK

SEQ ID NO: 247 - protein, 15 aa, Artificial sequence
  Peptide
GHPLGRCDIKDLPKE

SEQ ID NO: 248 - protein, 15 aa, Artificial sequence
  Peptide
HPLGRCDIKDLPKEI

SEQ ID NO: 249 - protein, 15 aa, Artificial sequence
  Peptide
PLGRCDIKDLPKEIT

SEQ ID NO: 250 - protein, 15 aa, Artificial sequence
  Peptide
LGRCDIKDLPKEITV

SEQ ID NO: 251 - protein, 15 aa, Artificial sequence
  Peptide
GRCDIKDLPKEITVA

SEQ ID NO: 252 - protein, 15 aa, Artificial sequence
  Peptide
TLSYYKLGASQRVGT

SEQ ID NO: 253 - protein, 15 aa, Artificial sequence
  Peptide
LSYYKLGASQRVGTD

SEQ ID NO: 254 - protein, 15 aa, Artificial sequence
  Peptide
SYYKLGASQRVGTDS

SEQ ID NO: 255 - protein, 15 aa, Artificial sequence
  Peptide
YYKLGASQRVGTDSG

SEQ ID NO: 256 - protein, 15 aa, Artificial sequence
  Peptide
YKLGASQRVGTDSGF

SEQ ID NO: 257 - protein, 15 aa, Artificial sequence
  Peptide
KLGASQRVGTDSGFA

SEQ ID NO: 258 - protein, 15 aa, Artificial sequence
  Peptide
LGASQRVGTDSGFAA

SEQ ID NO: 259 - protein, 15 aa, Artificial sequence
  Peptide
GASQRVGTDSGFAAY

SEQ ID NO: 260 - protein, 15 aa, Artificial sequence
  Peptide
ASQRVGTDSGFAAYN

SEQ ID NO: 261 - protein, 15 aa, Artificial sequence
  Peptide
IGNYKLNTDHAGSND

SEQ ID NO: 262 - protein, 15 aa, Artificial sequence
  Peptide
GNYKLNTDHAGSNDN

SEQ ID NO: 263 - protein, 15 aa, Artificial sequence
  Peptide
NYKLNTDHAGSNDNI

SEQ ID NO: 264 - protein, 15 aa, Artificial sequence
  Peptide
YKLNTDHAGSNDNIA

SEQ ID NO: 265 - protein, 15 aa, Artificial sequence
  Peptide
KLNTDHAGSNDNIAL

SEQ ID NO: 266 - protein, 15 aa, Artificial sequence
  Peptide
LNTDHAGSNDNIALL

SEQ ID NO: 267 - protein, 15 aa, Artificial sequence
  Peptide
NTDHAGSNDNIALLV

SEQ ID NO: 268 - protein, 15 aa, Artificial sequence
  Peptide
TDHAGSNDNIALLVQ

SEQ ID NO: 269 - protein, 15 aa, Artificial sequence
  Peptide
AEILIIIMRTFRIAI

SEQ ID NO: 270 - protein, 15 aa, Artificial sequence
  Peptide
EILIIIMRTFRIAIW

SEQ ID NO: 271 - protein, 15 aa, Artificial sequence
  Peptide
ILIIIMRTFRIAIWN

SEQ ID NO: 272 - protein, 15 aa, Artificial sequence
  Peptide
LIIIMRTFRIAIWNL

SEQ ID NO: 273 - protein, 15 aa, Artificial sequence
  Peptide
IIIMRTFRIAIWNLD

SEQ ID NO: 274 - protein, 15 aa, Artificial sequence
  Peptide
IIMRTFRIAIWNLDV

SEQ ID NO: 275 - protein, 15 aa, Artificial sequence
  Peptide
IMRTFRIAIWNLDVI

SEQ ID NO: 276 - protein, 15 aa, Artificial sequence
  Peptide
MRTFRIAIWNLDVII

SEQ ID NO: 277 - protein, 15 aa, Artificial sequence
  Peptide
RTFRIAIWNLDVIIS

SEQ ID NO: 278 - protein, 15 aa, Artificial sequence
  Peptide
VIISSIVRQLFKPLT

SEQ ID NO: 279 - protein, 15 aa, Artificial sequence
  Peptide
IISSIVRQLFKPLTK

SEQ ID NO: 280 - protein, 15 aa, Artificial sequence
  Peptide
ISSIVRQLFKPLTKK

SEQ ID NO: 281 - protein, 15 aa, Artificial sequence
  Peptide
SSIVRQLFKPLTKKN

SEQ ID NO: 282 - protein, 15 aa, Artificial sequence
  Peptide
SIVRQLFKPLTKKNY

SEQ ID NO: 283 - protein, 15 aa, Artificial sequence
  Peptide
IVRQLFKPLTKKNYS

SEQ ID NO: 284 - protein, 15 aa, Artificial sequence
  Peptide
VRQLFKPLTKKNYSE

SEQ ID NO: 285 - protein, 15 aa, Artificial sequence
  Peptide
RQLFKPLTKKNYSEL

SEQ ID NO: 286 - protein, 15 aa, Artificial sequence
  Peptide
QLFKPLTKKNYSELD

SEQ ID NO: 287 - protein, 15 aa, Artificial sequence
  Peptide
LFKPLTKKNYSELDD

SEQ ID NO: 288 - protein, 15 aa, Artificial sequence
  Peptide
FKPLTKKNYSELDDE

SEQ ID NO: 289 - protein, 15 aa, Artificial sequence
  Peptide
KPLTKKNYSELDDEE

SEQ ID NO: 290 - protein, 15 aa, Artificial sequence
  Peptide
PLTKKNYSELDDEEP

SEQ ID NO: 291 - protein, 15 aa, Artificial sequence
  Peptide
LTKKNYSELDDEEPM

SEQ ID NO: 292 - protein, 15 aa, Artificial sequence
  Peptide
TKKNYSELDDEEPME

SEQ ID NO: 293 - protein, 15 aa, Artificial sequence
  Peptide
KKNYSELDDEEPMEL

SEQ ID NO: 294 - protein, 15 aa, Artificial sequence
  Peptide
KNYSELDDEEPMELD

SEQ ID NO: 295 - protein, 15 aa, Artificial sequence
  Peptide
NYSELDDEEPMELDY

SEQ ID NO: 296 - protein, 15 aa, Artificial sequence
  Peptide
YSELDDEEPMELDYP

SEQ ID NO: 297 - protein, 15 aa, Artificial sequence
  Peptide
ELYHYQECVRGTTVL

SEQ ID NO: 298 - protein, 15 aa, Artificial sequence
  Peptide
LYHYQECVRGTTVLL

SEQ ID NO: 299 - protein, 15 aa, Artificial sequence
  Peptide
YHYQECVRGTTVLLK

SEQ ID NO: 300 - protein, 15 aa, Artificial sequence
  Peptide
HYQECVRGTTVLLKE

SEQ ID NO: 301 - protein, 15 aa, Artificial sequence
  Peptide
YQECVRGTTVLLKEP

SEQ ID NO: 302 - protein, 15 aa, Artificial sequence
  Peptide
QECVRGTTVLLKEPC

SEQ ID NO: 303 - protein, 15 aa, Artificial sequence
  Peptide
ECVRGTTVLLKEPCP

SEQ ID NO: 304 - protein, 15 aa, Artificial sequence
  Peptide
CVRGTTVLLKEPCPS

SEQ ID NO: 305 - protein, 15 aa, Artificial sequence
  Peptide
VRGTTVLLKEPCPSG

SEQ ID NO: 306 - protein, 15 aa, Artificial sequence
  Peptide
RGTTVLLKEPCPSGT

SEQ ID NO: 307 - protein, 15 aa, Artificial sequence
  Peptide
GTTVLLKEPCPSGTY

SEQ ID NO: 308 - protein, 15 aa, Artificial sequence
  Peptide
TTVLLKEPCPSGTYE

SEQ ID NO: 309 - protein, 15 aa, Artificial sequence
  Peptide
TVLLKEPCPSGTYEG

SEQ ID NO: 310 - protein, 15 aa, Artificial sequence
  Peptide
CPSGTYEGNSPFHPL

SEQ ID NO: 311 - protein, 15 aa, Artificial sequence
  Peptide
PSGTYEGNSPFHPLA

SEQ ID NO: 312 - protein, 15 aa, Artificial sequence
  Peptide
SGTYEGNSPFHPLAD

SEQ ID NO: 313 - protein, 15 aa, Artificial sequence
  Peptide
GTYEGNSPFHPLADN

SEQ ID NO: 314 - protein, 15 aa, Artificial sequence
  Peptide
TYEGNSPFHPLADNK

SEQ ID NO: 315 - protein, 15 aa, Artificial sequence
  Peptide
YEGNSPFHPLADNKF

SEQ ID NO: 316 - protein, 15 aa, Artificial sequence
  Peptide
EGNSPFHPLADNKFA

SEQ ID NO: 317 - protein, 15 aa, Artificial sequence
  Peptide
GNSPFHPLADNKFAL

SEQ ID NO: 318 - protein, 15 aa, Artificial sequence
  Peptide
NSPFHPLADNKFALT

SEQ ID NO: 319 - protein, 15 aa, Artificial sequence
  Peptide
SPFHPLADNKFALTC

SEQ ID NO: 320 - protein, 15 aa, Artificial sequence
  Peptide
PFHPLADNKFALTCT

SEQ ID NO: 321 - protein, 15 aa, Artificial sequence
  Peptide
FHPLADNKFALTCTS

SEQ ID NO: 322 - protein, 15 aa, Artificial sequence
  Peptide
HPLADNKFALTCTST

SEQ ID NO: 323 - protein, 15 aa, Artificial sequence
  Peptide
PLADNKFALTCTSTH

SEQ ID NO: 324 - protein, 15 aa, Artificial sequence
  Peptide
LADNKFALTCTSTHF

SEQ ID NO: 325 - protein, 15 aa, Artificial sequence
  Peptide
ADNKFALTCTSTHFA

SEQ ID NO: 326 - protein, 15 aa, Artificial sequence
  Peptide
DNKFALTCTSTHFAF

SEQ ID NO: 327 - protein, 15 aa, Artificial sequence
  Peptide
FIRQEEVQQELYSPL

SEQ ID NO: 328 - protein, 15 aa, Artificial sequence
  Peptide
IRQEEVQQELYSPLF

SEQ ID NO: 329 - protein, 15 aa, Artificial sequence
  Peptide
RQEEVQQELYSPLFL

SEQ ID NO: 330 - protein, 15 aa, Artificial sequence
  Peptide
QEEVQQELYSPLFLI

SEQ ID NO: 331 - protein, 15 aa, Artificial sequence
  Peptide
EEVQQELYSPLFLIV

SEQ ID NO: 332 - protein, 15 aa, Artificial sequence
  Peptide
EVQQELYSPLFLIVA

SEQ ID NO: 333 - protein, 15 aa, Artificial sequence
  Peptide
VQQELYSPLFLIVAA

SEQ ID NO: 334 - protein, 15 aa, Artificial sequence
  Peptide
RWHTMVQTCTPNVTI

SEQ ID NO: 335 - protein, 15 aa, Artificial sequence
  Peptide
WHTMVQTCTPNVTIN

SEQ ID NO: 336 - protein, 15 aa, Artificial sequence
  Peptide
HTMVQTCTPNVTINC

SEQ ID NO: 337 - protein, 15 aa, Artificial sequence
  Peptide
TMVQTCTPNVTINCQ

SEQ ID NO: 338 - protein, 15 aa, Artificial sequence
  Peptide
MVQTCTPNVTINCQD

SEQ ID NO: 339 - protein, 15 aa, Artificial sequence
  Peptide
PNVTINCQDPAGGAL

SEQ ID NO: 340 - protein, 15 aa, Artificial sequence
  Peptide
NVTINCQDPAGGALI

SEQ ID NO: 341 - protein, 15 aa, Artificial sequence
  Peptide
VTINCQDPAGGALIA

SEQ ID NO: 342 - protein, 15 aa, Artificial sequence
  Peptide
TINCQDPAGGALIAR

SEQ ID NO: 343 - protein, 15 aa, Artificial sequence
  Peptide
INCQDPAGGALIARC

SEQ ID NO: 344 - protein, 15 aa, Artificial sequence
  Peptide
NCQDPAGGALIARCW

SEQ ID NO: 345 - protein, 15 aa, Artificial sequence
  Peptide
CQDPAGGALIARCWY

SEQ ID NO: 346 - protein, 15 aa, Artificial sequence
  Peptide
QDPAGGALIARCWYL

SEQ ID NO: 347 - protein, 15 aa, Artificial sequence
  Peptide
IARCWYLHEGHQTAA

SEQ ID NO: 348 - protein, 15 aa, Artificial sequence
  Peptide
ARCWYLHEGHQTAAF

SEQ ID NO: 349 - protein, 15 aa, Artificial sequence
  Peptide
RCWYLHEGHQTAAFR

SEQ ID NO: 350 - protein, 15 aa, Artificial sequence
  Peptide
CWYLHEGHQTAAFRD

SEQ ID NO: 351 - protein, 15 aa, Artificial sequence
  Peptide
WYLHEGHQTAAFRDV

SEQ ID NO: 352 - protein, 15 aa, Artificial sequence
  Peptide
YLHEGHQTAAFRDVL

SEQ ID NO: 353 - protein, 15 aa, Artificial sequence
  Peptide
LHEGHQTAAFRDVLV

SEQ ID NO: 354 - protein, 15 aa, Artificial sequence
  Peptide
HEGHQTAAFRDVLVV

SEQ ID NO: 355 - protein, 15 aa, Artificial sequence
  Peptide
EGHQTAAFRDVLVVL

SEQ ID NO: 356 - protein, 15 aa, Artificial sequence
  Peptide
GHQTAAFRDVLVVLN

SEQ ID NO: 357 - protein, 15 aa, Artificial sequence
  Peptide
HQTAAFRDVLVVLNK

SEQ ID NO: 358 - protein, 15 aa, Artificial sequence
  Peptide
NNAATVLQLPQGTTL

SEQ ID NO: 359 - protein, 15 aa, Artificial sequence
  Peptide
NAATVLQLPQGTTLP

SEQ ID NO: 360 - protein, 15 aa, Artificial sequence
  Peptide
AATVLQLPQGTTLPK

SEQ ID NO: 361 - protein, 15 aa, Artificial sequence
  Peptide
ATVLQLPQGTTLPKG

SEQ ID NO: 362 - protein, 15 aa, Artificial sequence
  Peptide
TVLQLPQGTTLPKGF

SEQ ID NO: 363 - protein, 15 aa, Artificial sequence
  Peptide
VLQLPQGTTLPKGFY

SEQ ID NO: 364 - protein, 15 aa, Artificial sequence
  Peptide
LQLPQGTTLPKGFYA

SEQ ID NO: 365 - protein, 15 aa, Artificial sequence
  Peptide
QLPQGTTLPKGFYAE

SEQ ID NO: 366 - protein, 15 aa, Artificial sequence
  Peptide
LPQGTTLPKGFYAEG

SEQ ID NO: 367 - protein, 15 aa, Artificial sequence
  Peptide
PQGTTLPKGFYAEGS

SEQ ID NO: 368 - protein, 15 aa, Artificial sequence
  Peptide
QGTTLPKGFYAEGSR

SEQ ID NO: 369 - protein, 15 aa, Artificial sequence
  Peptide
GTTLPKGFYAEGSRG

SEQ ID NO: 370 - protein, 15 aa, Artificial sequence
  Peptide
TTLPKGFYAEGSRGG

SEQ ID NO: 371 - protein, 15 aa, Artificial sequence
  Peptide
TLPKGFYAEGSRGGS

SEQ ID NO: 372 - protein, 15 aa, Artificial sequence
  Peptide
NSPARMASGGGETAL

SEQ ID NO: 373 - protein, 15 aa, Artificial sequence
  Peptide
SPARMASGGGETALA

SEQ ID NO: 374 - protein, 15 aa, Artificial sequence
  Peptide
PARMASGGGETALAL

SEQ ID NO: 375 - protein, 15 aa, Artificial sequence
  Peptide
ARMASGGGETALALL

SEQ ID NO: 376 - protein, 15 aa, Artificial sequence
  Peptide
RMASGGGETALALLL

SEQ ID NO: 377 - protein, 15 aa, Artificial sequence
  Peptide
MASGGGETALALLLL

SEQ ID NO: 378 - protein, 15 aa, Artificial sequence
  Peptide
ASGGGETALALLLLD

SEQ ID NO: 379 - protein, 15 aa, Artificial sequence
  Peptide
QQGQTVTKKSAAEAS

SEQ ID NO: 380 - protein, 15 aa, Artificial sequence
  Peptide
QGQTVTKKSAAEASK

SEQ ID NO: 381 - protein, 15 aa, Artificial sequence
  Peptide
GQTVTKKSAAEASKK

SEQ ID NO: 382 - protein, 15 aa, Artificial sequence
  Peptide
QTVTKKSAAEASKKP

SEQ ID NO: 383 - protein, 15 aa, Artificial sequence
  Peptide
TVTKKSAAEASKKPR

SEQ ID NO: 384 - protein, 15 aa, Artificial sequence
  Peptide
VTKKSAAEASKKPRQ

SEQ ID NO: 385 - protein, 15 aa, Artificial sequence
  Peptide
TKKSAAEASKKPRQK

SEQ ID NO: 386 - protein, 15 aa, Artificial sequence
  Peptide
KKSAAEASKKPRQKR

SEQ ID NO: 387 - protein, 15 aa, Artificial sequence
  Peptide
KSAAEASKKPRQKRT

SEQ ID NO: 388 - protein, 15 aa, Artificial sequence
  Peptide
SAAEASKKPRQKRTA

SEQ ID NO: 389 - protein, 15 aa, Artificial sequence
  Peptide
AAEASKKPRQKRTAT

SEQ ID NO: 390 - protein, 15 aa, Artificial sequence
  Peptide
KPRQKRTATKQYNVT

SEQ ID NO: 391 - protein, 15 aa, Artificial sequence
  Peptide
PRQKRTATKQYNVTQ

SEQ ID NO: 392 - protein, 15 aa, Artificial sequence
  Peptide
RQKRTATKQYNVTQA

SEQ ID NO: 393 - protein, 15 aa, Artificial sequence
  Peptide
QKRTATKQYNVTQAF

SEQ ID NO: 394 - protein, 15 aa, Artificial sequence
  Peptide
KRTATKQYNVTQAFG

SEQ ID NO: 395 - protein, 15 aa, Artificial sequence
  Peptide
RTATKQYNVTQAFGR

SEQ ID NO: 396 - protein, 15 aa, Artificial sequence
  Peptide
TATKQYNVTQAFGRR

SEQ ID NO: 397 - protein, 15 aa, Artificial sequence
  Peptide
FGRRGPEQTQGNFGD

SEQ ID NO: 398 - protein, 15 aa, Artificial sequence
  Peptide
GRRGPEQTQGNFGDQ

SEQ ID NO: 399 - protein, 15 aa, Artificial sequence
  Peptide
RRGPEQTQGNFGDQD

SEQ ID NO: 400 - protein, 15 aa, Artificial sequence
  Peptide
RGPEQTQGNFGDQDL

SEQ ID NO: 401 - protein, 15 aa, Artificial sequence
  Peptide
GPEQTQGNFGDQDLI

SEQ ID NO: 402 - protein, 15 aa, Artificial sequence
  Peptide
PEQTQGNFGDQDLIR

SEQ ID NO: 403 - protein, 15 aa, Artificial sequence
  Peptide
EQTQGNFGDQDLIRQ

SEQ ID NO: 404 - protein, 15 aa, Artificial sequence
  Peptide
QTQGNFGDQDLIRQG

SEQ ID NO: 405 - protein, 15 aa, Artificial sequence
  Peptide
IKLDDKDPQFKDNVI

SEQ ID NO: 406 - protein, 15 aa, Artificial sequence
  Peptide
KLDDKDPQFKDNVIL

SEQ ID NO: 407 - protein, 15 aa, Artificial sequence
  Peptide
LDDKDPQFKDNVILL

SEQ ID NO: 408 - protein, 15 aa, Artificial sequence
  Peptide
DDKDPQFKDNVILLN

SEQ ID NO: 409 - protein, 15 aa, Artificial sequence
  Peptide
DKDPQFKDNVILLNK

SEQ ID NO: 410 - protein, 15 aa, Artificial sequence
  Peptide
KDPQFKDNVILLNKH

SEQ ID NO: 411 - protein, 15 aa, Artificial sequence
  Peptide
DPQFKDNVILLNKHI

SEQ ID NO: 412 - protein, 15 aa, Artificial sequence
  Peptide
PQFKDNVILLNKHID

SEQ ID NO: 413 - protein, 15 aa, Artificial sequence
  Peptide
QFKDNVILLNKHIDA

SEQ ID NO: 414 - protein, 15 aa, Artificial sequence
  Peptide
QPLPQRQKKQPTVTL

SEQ ID NO: 415 - protein, 15 aa, Artificial sequence
  Peptide
PLPQRQKKQPTVTLL

SEQ ID NO: 416 - protein, 15 aa, Artificial sequence
  Peptide
LPQRQKKQPTVTLLP

SEQ ID NO: 417 - protein, 15 aa, Artificial sequence
  Peptide
PQRQKKQPTVTLLPA

SEQ ID NO: 418 - protein, 15 aa, Artificial sequence
  Peptide
QRQKKQPTVTLLPAA

SEQ ID NO: 419 - protein, 15 aa, Artificial sequence
  Peptide
RQKKQPTVTLLPAAD

SEQ ID NO: 420 - protein, 15 aa, Artificial sequence
  Peptide
QKKQPTVTLLPAADM

SEQ ID NO: 421 - protein, 15 aa, Artificial sequence
  Peptide
DNASPASTVHATATI

SEQ ID NO: 422 - protein, 15 aa, Artificial sequence
  Peptide
NASPASTVHATATIP

SEQ ID NO: 423 - protein, 15 aa, Artificial sequence
  Peptide
ASPASTVHATATIPL

SEQ ID NO: 424 - protein, 15 aa, Artificial sequence
  Peptide
SPASTVHATATIPLQ

SEQ ID NO: 425 - protein, 15 aa, Artificial sequence
  Peptide
PASTVHATATIPLQA

SEQ ID NO: 426 - protein, 15 aa, Artificial sequence
  Peptide
ASTVHATATIPLQAS

SEQ ID NO: 427 - protein, 15 aa, Artificial sequence
  Peptide
STVHATATIPLQASL

SEQ ID NO: 428 - protein, 15 aa, Artificial sequence
  Peptide
TVHATATIPLQASLP

SEQ ID NO: 429 - protein, 15 aa, Artificial sequence
  Peptide
VHATATIPLQASLPF

SEQ ID NO: 430 - protein, 15 aa, Artificial sequence
  Peptide
AVFQSATKIIALNKR

SEQ ID NO: 431 - protein, 15 aa, Artificial sequence
  Peptide
VFQSATKIIALNKRW

SEQ ID NO: 432 - protein, 15 aa, Artificial sequence
  Peptide
FQSATKIIALNKRWQ

SEQ ID NO: 433 - protein, 15 aa, Artificial sequence
  Peptide
QSATKIIALNKRWQL

SEQ ID NO: 434 - protein, 15 aa, Artificial sequence
  Peptide
SATKIIALNKRWQLA

SEQ ID NO: 435 - protein, 15 aa, Artificial sequence
  Peptide
ATKIIALNKRWQLAL

SEQ ID NO: 436 - protein, 15 aa, Artificial sequence
  Peptide
TKIIALNKRWQLALY

SEQ ID NO: 437 - protein, 15 aa, Artificial sequence
  Peptide
KIIALNKRWQLALYK

SEQ ID NO: 438 - protein, 15 aa, Artificial sequence
  Peptide
IIALNKRWQLALYKG

SEQ ID NO: 439 - protein, 15 aa, Artificial sequence
  Peptide
IALNKRWQLALYKGF

SEQ ID NO: 440 - protein, 15 aa, Artificial sequence
  Peptide
MMPTTLFAGTHITMT

SEQ ID NO: 441 - protein, 15 aa, Artificial sequence
  Peptide
MPTTLFAGTHITMTT

SEQ ID NO: 442 - protein, 15 aa, Artificial sequence
  Peptide
PTTLFAGTHITMTTV

SEQ ID NO: 443 - protein, 15 aa, Artificial sequence
  Peptide
TTLFAGTHITMTTVY

SEQ ID NO: 444 - protein, 15 aa, Artificial sequence
  Peptide
TLFAGTHITMTTVYH

SEQ ID NO: 445 - protein, 15 aa, Artificial sequence
  Peptide
LFAGTHITMTTVYHI

SEQ ID NO: 446 - protein, 15 aa, Artificial sequence
  Peptide
FAGTHITMTTVYHIT

SEQ ID NO: 447 - protein, 15 aa, Artificial sequence
  Peptide
AGTHITMTTVYHITV

SEQ ID NO: 448 - protein, 15 aa, Artificial sequence
  Peptide
GTHITMTTVYHITVS

SEQ ID NO: 449 - protein, 15 aa, Artificial sequence
  Peptide
GTITVEELKQLLEQW

SEQ ID NO: 450 - protein, 15 aa, Artificial sequence
  Peptide
TITVEELKQLLEQWN

SEQ ID NO: 451 - protein, 15 aa, Artificial sequence
  Peptide
ITVEELKQLLEQWNL

SEQ ID NO: 452 - protein, 15 aa, Artificial sequence
  Peptide
TVEELKQLLEQWNLV

SEQ ID NO: 453 - protein, 15 aa, Artificial sequence
  Peptide
VEELKQLLEQWNLVI

SEQ ID NO: 454 - protein, 15 aa, Artificial sequence
  Peptide
EELKQLLEQWNLVIG

SEQ ID NO: 455 - protein, 15 aa, Artificial sequence
  Peptide
VIGAVIIRGHLRMAG

SEQ ID NO: 456 - protein, 15 aa, Artificial sequence
  Peptide
IGAVIIRGHLRMAGH

SEQ ID NO: 457 - protein, 15 aa, Artificial sequence
  Peptide
GAVIIRGHLRMAGHP

SEQ ID NO: 458 - protein, 15 aa, Artificial sequence
  Peptide
AVIIRGHLRMAGHPL

SEQ ID NO: 459 - protein, 15 aa, Artificial sequence
  Peptide
VIIRGHLRMAGHPLG

SEQ ID NO: 460 - protein, 15 aa, Artificial sequence
  Peptide
IIRGHLRMAGHPLGR

SEQ ID NO: 461 - protein, 15 aa, Artificial sequence
  Peptide
IRGHLRMAGHPLGRC

SEQ ID NO: 462 - protein, 15 aa, Artificial sequence
  Peptide
RGHLRMAGHPLGRCD

SEQ ID NO: 463 - protein, 15 aa, Artificial sequence
  Peptide
GHLRMAGHPLGRCDI

SEQ ID NO: 464 - protein, 15 aa, Artificial sequence
  Peptide
HLRMAGHPLGRCDIK

SEQ ID NO: 465 - protein, 15 aa, Artificial sequence
  Peptide
TYEGNSPFHPLADNK

SEQ ID NO: 466 - protein, 15 aa, Artificial sequence
  Peptide
YEGNSPFHPLADNKF

SEQ ID NO: 467 - protein, 15 aa, Artificial sequence
  Peptide
EGNSPFHPLADNKFA

SEQ ID NO: 468 - protein, 15 aa, Artificial sequence
  Peptide
GNSPFHPLADNKFAL

SEQ ID NO: 469 - protein, 15 aa, Artificial sequence
  Peptide
NSPFHPLADNKFALT

SEQ ID NO: 470 - protein, 15 aa, Artificial sequence
  Peptide
SPFHPLADNKFALTC

SEQ ID NO: 471 - protein, 15 aa, Artificial sequence
  Peptide
IARCWYLHEGHQTAA

SEQ ID NO: 472 - protein, 15 aa, Artificial sequence
  Peptide
ARCWYLHEGHQTAAF

SEQ ID NO: 473 - protein, 15 aa, Artificial sequence
  Peptide
RCWYLHEGHQTAAFR

SEQ ID NO: 474 - protein, 15 aa, Artificial sequence
  Peptide
CWYLHEGHQTAAFRD

SEQ ID NO: 475 - protein, 15 aa, Artificial sequence
  Peptide
WYLHEGHQTAAFRDV

SEQ ID NO: 476 - protein, 15 aa, Artificial sequence
  Peptide
YLHEGHQTAAFRDVL

SEQ ID NO: 477 - protein, 15 aa, Artificial sequence
  Peptide
LHEGHQTAAFRDVLV

SEQ ID NO: 478 - protein, 15 aa, Artificial sequence
  Peptide
HEGHQTAAFRDVLVV

SEQ ID NO: 479 - protein, 15 aa, Artificial sequence
  Peptide
AATVLQLPQGTTLPK

SEQ ID NO: 480 - protein, 15 aa, Artificial sequence
  Peptide
ATVLQLPQGTTLPKG

SEQ ID NO: 481 - protein, 15 aa, Artificial sequence
  Peptide
TVLQLPQGTTLPKGF

SEQ ID NO: 482 - protein, 15 aa, Artificial sequence
  Peptide
NSTPGSSRGNSPARM

SEQ ID NO: 483 - protein, 15 aa, Artificial sequence
  Peptide
STPGSSRGNSPARMA

SEQ ID NO: 484 - protein, 15 aa, Artificial sequence
  Peptide
TPGSSRGNSPARMAS

SEQ ID NO: 485 - protein, 15 aa, Artificial sequence
  Peptide
PGSSRGNSPARMASG

SEQ ID NO: 486 - protein, 15 aa, Artificial sequence
  Peptide
GSSRGNSPARMASGG

SEQ ID NO: 487 - protein, 15 aa, Artificial sequence
  Peptide
LDDKDPQFKDNVILL

SEQ ID NO: 488 - protein, 15 aa, Artificial sequence
  Peptide
DDKDPQFKDNVILLN

SEQ ID NO: 489 - protein, 15 aa, Artificial sequence
  Peptide
DKDPQFKDNVILLNK

SEQ ID NO: 490 - protein, 15 aa, Artificial sequence
  Peptide
KDPQFKDNVILLNKH

SEQ ID NO: 491 - protein, 15 aa, Artificial sequence
  Peptide
DPQFKDNVILLNKHI

SEQ ID NO: 492 - protein, 15 aa, Artificial sequence
  Peptide
AVFQSATKIIALNKR

SEQ ID NO: 493 - protein, 15 aa, Artificial sequence
  Peptide
VFQSATKIIALNKRW

SEQ ID NO: 494 - protein, 15 aa, Artificial sequence
  Peptide
FQSATKIIALNKRWQ

SEQ ID NO: 495 - protein, 15 aa, Artificial sequence
  Peptide
QSATKIIALNKRWQL

SEQ ID NO: 496 - protein, 15 aa, Artificial sequence
  Peptide
SATKIIALNKRWQLA

SEQ ID NO: 497 - protein, 15 aa, Artificial sequence
  Peptide
ATKIIALNKRWQLAL

SEQ ID NO: 498 - protein, 15 aa, Artificial sequence
  Peptide
TKIIALNKRWQLALY

SEQ ID NO: 499 - protein, 15 aa, Artificial sequence
  Peptide
KIIALNKRWQLALYK

SEQ ID NO: 500 - protein, 15 aa, Artificial sequence
  Peptide
IIALNKRWQLALYKG

SEQ ID NO: 501 - protein, 15 aa, Artificial sequence
  Peptide
IALNKRWQLALYKGF

SEQ ID NO: 502 - protein, 15 aa, Artificial sequence
  Peptide
ALNKRWQLALYKGFQ

SEQ ID NO: 503 - protein, 15 aa, Artificial sequence
  Peptide
LNKRWQLALYKGFQF

SEQ ID NO: 504 - protein, 15 aa, Artificial sequence
  Peptide
NKRWQLALYKGFQFI

SEQ ID NO: 505 - protein, 15 aa, Artificial sequence
  Peptide
LQCINACRIIMRCWL

SEQ ID NO: 506 - protein, 15 aa, Artificial sequence
  Peptide
QCINACRIIMRCWLC

SEQ ID NO: 507 - protein, 15 aa, Artificial sequence
  Peptide
CINACRIIMRCWLCW

SEQ ID NO: 508 - protein, 15 aa, Artificial sequence
  Peptide
INACRIIMRCWLCWK

SEQ ID NO: 509 - protein, 15 aa, Artificial sequence
  Peptide
NACRIIMRCWLCWKC

SEQ ID NO: 510 - protein, 15 aa, Artificial sequence
  Peptide
ACRIIMRCWLCWKCK

SEQ ID NO: 511 - protein, 15 aa, Artificial sequence
  Peptide
TAFQHQNSKKTTKLV

SEQ ID NO: 512 - protein, 15 aa, Artificial sequence
  Peptide
AFQHQNSKKTTKLVV

SEQ ID NO: 513 - protein, 15 aa, Artificial sequence
  Peptide
FQHQNSKKTTKLVVI

SEQ ID NO: 514 - protein, 15 aa, Artificial sequence
  Peptide
QHQNSKKTTKLVVIL

SEQ ID NO: 515 - protein, 15 aa, Artificial sequence
  Peptide
HQNSKKTTKLVVILR

SEQ ID NO: 516 - protein, 15 aa, Artificial sequence
  Peptide
QNSKKTTKLVVILRI

SEQ ID NO: 517 - protein, 15 aa, Artificial sequence
  Peptide
NSKKTTKLVVILRIG

SEQ ID NO: 518 - protein, 15 aa, Artificial sequence
  Peptide
SKKTTKLVVILRIGT

SEQ ID NO: 519 - protein, 15 aa, Artificial sequence
  Peptide
KKTTKLVVILRIGTQ

SEQ ID NO: 520 - protein, 15 aa, Artificial sequence
  Peptide
KTTKLVVILRIGTQV

SEQ ID NO: 521 - protein, 15 aa, Artificial sequence
  Peptide
TTKLVVILRIGTQVL

SEQ ID NO: 522 - protein, 15 aa, Artificial sequence
  Peptide
MADNGTITVEELKQL

SEQ ID NO: 523 - protein, 15 aa, Artificial sequence
  Peptide
ADNGTITVEELKQLL

SEQ ID NO: 524 - protein, 15 aa, Artificial sequence
  Peptide
DNGTITVEELKQLLE

SEQ ID NO: 525 - protein, 15 aa, Artificial sequence
  Peptide
NGTITVEELKQLLEQ

SEQ ID NO: 526 - protein, 15 aa, Artificial sequence
  Peptide
GTITVEELKQLLEQW

SEQ ID NO: 527 - protein, 15 aa, Artificial sequence
  Peptide
TITVEELKQLLEQWN

SEQ ID NO: 528 - protein, 15 aa, Artificial sequence
  Peptide
ITVEELKQLLEQWNL

SEQ ID NO: 529 - protein, 15 aa, Artificial sequence
  Peptide
TVEELKQLLEQWNLV

SEQ ID NO: 530 - protein, 15 aa, Artificial sequence
  Peptide
VEELKQLLEQWNLVI

SEQ ID NO: 531 - protein, 15 aa, Artificial sequence
  Peptide
FACADGTRHTYQLRA

SEQ ID NO: 532 - protein, 15 aa, Artificial sequence
  Peptide
ACADGTRHTYQLRAR

SEQ ID NO: 533 - protein, 15 aa, Artificial sequence
  Peptide
CADGTRHTYQLRARS

SEQ ID NO: 534 - protein, 15 aa, Artificial sequence
  Peptide
ADGTRHTYQLRARSV

SEQ ID NO: 535 - protein, 15 aa, Artificial sequence
  Peptide
DGTRHTYQLRARSVS

SEQ ID NO: 536 - protein, 15 aa, Artificial sequence
  Peptide
GTRHTYQLRARSVSP

SEQ ID NO: 537 - protein, 15 aa, Artificial sequence
  Peptide
TRHTYQLRARSVSPK

SEQ ID NO: 538 - protein, 15 aa, Artificial sequence
  Peptide
RHTYQLRARSVSPKL

SEQ ID NO: 539 - protein, 15 aa, Artificial sequence
  Peptide
HTYQLRARSVSPKLF

SEQ ID NO: 540 - protein, 15 aa, Artificial sequence
  Peptide
TYQLRARSVSPKLFI

SEQ ID NO: 541 - protein, 15 aa, Artificial sequence
  Peptide
YQLRARSVSPKLFIR

SEQ ID NO: 542 - protein, 15 aa, Artificial sequence
  Peptide
QLRARSVSPKLFIRQ

SEQ ID NO: 543 - protein, 15 aa, Artificial sequence
  Peptide
LRARSVSPKLFIRQE

SEQ ID NO: 544 - protein, 15 aa, Artificial sequence
  Peptide
RARSVSPKLFIRQEE

SEQ ID NO: 545 - protein, 15 aa, Artificial sequence
  Peptide
SGPDDQIGYYRRATR

SEQ ID NO: 546 - protein, 15 aa, Artificial sequence
  Peptide
GPDDQIGYYRRATRR

SEQ ID NO: 547 - protein, 15 aa, Artificial sequence
  Peptide
PDDQIGYYRRATRRV

SEQ ID NO: 548 - protein, 15 aa, Artificial sequence
  Peptide
DDQIGYYRRATRRVR

SEQ ID NO: 549 - protein, 15 aa, Artificial sequence
  Peptide
DQIGYYRRATRRVRG

SEQ ID NO: 550 - protein, 15 aa, Artificial sequence
  Peptide
QIGYYRRATRRVRGG

SEQ ID NO: 551 - protein, 15 aa, Artificial sequence
  Peptide
IGYYRRATRRVRGGD

SEQ ID NO: 552 - protein, 15 aa, Artificial sequence
  Peptide
GYYRRATRRVRGGDG

SEQ ID NO: 553 - protein, 15 aa, Artificial sequence
  Peptide
RNSTPGSSRGNSPAR

SEQ ID NO: 554 - protein, 15 aa, Artificial sequence
  Peptide
NSTPGSSRGNSPARM

SEQ ID NO: 555 - protein, 15 aa, Artificial sequence
  Peptide
STPGSSRGNSPARMA

SEQ ID NO: 556 - protein, 15 aa, Artificial sequence
  Peptide
TPGSSRGNSPARMAS

SEQ ID NO: 557 - protein, 15 aa, Artificial sequence
  Peptide
PGSSRGNSPARMASG

SEQ ID NO: 558 - protein, 15 aa, Artificial sequence
  Peptide
GSSRGNSPARMASGG

SEQ ID NO: 559 - protein, 15 aa, Artificial sequence
  Peptide
PRQKRTATKQYNVTQ

SEQ ID NO: 560 - protein, 15 aa, Artificial sequence
  Peptide
RQKRTATKQYNVTQA

SEQ ID NO: 561 - protein, 15 aa, Artificial sequence
  Peptide
QKRTATKQYNVTQAF

SEQ ID NO: 562 - protein, 15 aa, Artificial sequence
  Peptide
KRTATKQYNVTQAFG

SEQ ID NO: 563 - protein, 15 aa, Artificial sequence
  Peptide
RTATKQYNVTQAFGR

SEQ ID NO: 564 - protein, 15 aa, Artificial sequence
  Peptide
TATKQYNVTQAFGRR

SEQ ID NO: 565 - protein, 15 aa, Artificial sequence
  Peptide
ATKQYNVTQAFGRRG

SEQ ID NO: 566 - protein, 15 aa, Artificial sequence
  Peptide
TKQYNVTQAFGRRGP

SEQ ID NO: 567 - protein, 15 aa, Artificial sequence
  Peptide
KQYNVTQAFGRRGPE

SEQ ID NO: 568 - protein, 15 aa, Artificial sequence
  Peptide
QYNVTQAFGRRGPEQ

SEQ ID NO: 569 - protein, 15 aa, Artificial sequence
  Peptide
YNVTQAFGRRGPEQT

SEQ ID NO: 570 - protein, 15 aa, Artificial sequence
  Peptide
NVTQAFGRRGPEQTQ

SEQ ID NO: 571 - protein, 15 aa, Artificial sequence
  Peptide
VTQAFGRRGPEQTQG

SEQ ID NO: 572 - protein, 15 aa, Artificial sequence
  Peptide
TQAFGRRGPEQTQGN

SEQ ID NO: 573 - protein, 243 aa, Artificial sequence
  SC03-001
SMAEVQLVESGGGLVKPGGSLRLSCAASGFTFSGYSMNWVRQAPGKGLEWVSSISGGSTYYADSRKGRFTISRDNSKNTLYLQMNNLRAEDTAVYYCARHRFRHVFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 574 - protein, 249 aa, Artificial sequence
  SC03-002
SMAEVQLVESGGGLVKPGGSLRLSCAASGFTFSGYSMSWVRQAPGKGLEWVGRTRNKANSYTTEYAASVKGRFTISRDDSKNSLYLQMNSLKTEDTAVYYCARYYSRSLKAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 575 - protein, 245 aa, Artificial sequence
  SC03-003
SMAEVQLVESGGGLVQPGGSLRLSCAASGFTFSSYPMNWVRQAPGKGLEWVSAISGSGGSTYYADSVKGRFTISRDNSKNTLYLQMNSLRAEDTAVYYCARRSYFRRFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 576 - protein, 245 aa, Artificial sequence
  SC03-005
SMAEVQLVESGGGLIQPGGSLRLSCAASGFTFSGYTMSWVRQAPGQGLEWVSSISGGSTYYADSRKGRFTISRDNSKNTLYLQMNNLRAEDTAVYYCAKGGGRPYNPFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 577 - protein, 251 aa, Artificial sequence
  SC03-006
SMAEVQLVESGGGLVQPGGSLRLSCAASGFTFSGYPMHWVRQAPGKGLEWVAVISYDGSNKYYADSVKGRFTISRDNSKNTLYLQMNSLRAEDTAVYYCAKDGSPRTPSFDYWGQGTLVTVLEGTGGSGGTGSGTGTSELDIQMTQSPHSLSASVGDRVTITCRASQGISNYLAWYQQKPGKVPKLLIYAASTLQSGVPSRFSGSGSGTDFTLTISSLQPEDVGVYYCQQRFRTPVTFGQGTKLEIKRAAA

SEQ ID NO: 578 - protein, 247 aa, Artificial sequence
  SC03-007
SMAEVQLVESGGGLVQPRGSLRLSCAASGFTFSDYRMNWVRQAPGKGLERVAVISYDGSNKYYADSVKGRFTISRDNSKNTLYLQMNSLRAEDTAVYYCARGYWTSLTGFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 579 - protein, 245 aa, Artificial sequence
  SC03-008
SMAEVQLVESGGGVVQPGRSLRLSCAASGFTFSSYPMNWVRQAPGKGLEWVSAISGSGGSTYYADSVKGRFTISRDNSKNTLYLQMNSLRAEDTAVYYCARRVRPRRFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 580 - protein, 248 aa, Artificial sequence
  SC03-009
SMAEVQLVESGGGVVQPGRSLRLSCAASGFTFSDYPMNWVRQAPGKGLEWVSSISGSGGSTYYADSVKGRFTISRDNSKNTLYLQMNSLRAEDTAVYYCAKGLFMVTTYAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 581 - protein, 245 aa, Artificial sequence
  SC03-010
SMAEVQLVESGGGVVQPGRSLRLSCATSGFTFSGYTMHWVRQAPGKGLEWVSSISGGSTYYADSRKGRFTISRDNSKNTLYLQMNNLRAEDTAVYYCAKGGGLPYLSFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 582 - protein, 247 aa, Artificial sequence
  SC03-012
AMAQVQLVQSGAEVKKPGASVKVSCKASGYTFTSYGISWVRQAPGQGLEWMGWISAYNGNTNYAQKLQGRVTMTTDTSTSTAYMELSSLRSDDTAVYYCARMFRKSSFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 583 - protein, 249 aa, Artificial sequence
  SC03-013
AMAEVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAKGLTPLYFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 584 - protein, 249 aa, Artificial sequence
  SC03-014
AMAEVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGISPFYFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 585 - protein, 244 aa, Artificial sequence
  SC03-015
AMAEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGLSLRPWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 586 - protein, 247 aa, Artificial sequence
  SC03-018
AMAEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKFNPFTSFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 587 - protein, 9 aa, Artificial sequence
  CDR3 of SC03-018
FNPFTSFDY

SEQ ID NO: 588 - DNA, 1344 bp, Artificial sequence
  CDS (1)..(1344): Heavy chain of 03-018
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaaagtttaatccgtttacttcctttgactactggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 589 - protein, 448 aa, Artificial sequence
  Heavy chain of 03-018
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKFNPFTSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 590 - DNA, 642 bp, Artificial sequence
  CDS (1)..(642): Light chain of 03-018
gacattcagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaacggaccgtggccgctcccagcgtgttcatcttccccccctccgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggactccacctacagcctgagcagcaccctcaccctgagcaaggccgactacgagaagcacaaggtgtacgcctgcgaggtgacccaccagggcctgagcagccccgtgaccaagagcttcaaccggggcgagtgt

SEQ ID NO: 591 - protein, 214 aa, Artificial sequence
  Light chain of 03-018
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 592 - protein, 15 aa, Artificial sequence
  Peptide
NGPQSNQRSAPRITF

SEQ ID NO: 593 - protein, 15 aa, Artificial sequence
  Peptide
GPQSNQRSAPRITFG

SEQ ID NO: 594 - protein, 15 aa, Artificial sequence
  Peptide
PQSNQRSAPRITFGG

SEQ ID NO: 595 - protein, 15 aa, Artificial sequence
  Peptide
QSNQRSAPRITFGGP

SEQ ID NO: 596 - protein, 15 aa, Artificial sequence
  Peptide
SNQRSAPRITFGGPT

SEQ ID NO: 597 - protein, 15 aa, Artificial sequence
  Peptide
NQRSAPRITFGGPTD

SEQ ID NO: 598 - protein, 15 aa, Artificial sequence
  Peptide
QRSAPRITFGGPTDS

SEQ ID NO: 599 - protein, 15 aa, Artificial sequence
  Peptide
RSAPRITFGGPTDST

SEQ ID NO: 600 - protein, 15 aa, Artificial sequence
  Peptide
SAPRITFGGPTDSTD

SEQ ID NO: 601 - protein, 15 aa, Artificial sequence
  Peptide
APRITFGGPTDSTDN

SEQ ID NO: 602 - protein, 15 aa, Artificial sequence
  Peptide
PRITFGGPTDSTDNN

SEQ ID NO: 603 - protein, 15 aa, Artificial sequence
  Peptide
RITFGGPTDSTDNNQ

SEQ ID NO: 604 - protein, 15 aa, Artificial sequence
  Peptide
ITFGGPTDSTDNNQN

SEQ ID NO: 605 - protein, 9 aa, Artificial sequence
  Peptide
RSAPRITFG

SEQ ID NO: 606 - protein, 7 aa, Artificial sequence
  Peptide
QGTTLPK

SEQ ID NO: 607 - protein, 15 aa, Artificial sequence
  Peptide
MDLFMRFFTLGSITA

SEQ ID NO: 608 - protein, 15 aa, Artificial sequence
  Peptide
DLFMRFFTLGSITAQ

SEQ ID NO: 609 - protein, 15 aa, Artificial sequence
  Peptide
LFMRFFTLGSITAQP

SEQ ID NO: 610 - protein, 15 aa, Artificial sequence
  Peptide
FMRFFTLGSITAQPV

SEQ ID NO: 611 - protein, 15 aa, Artificial sequence
  Peptide
MRFFTLGSITAQPVK

SEQ ID NO: 612 - protein, 15 aa, Artificial sequence
  Peptide
HATATIPLQASLPFG

SEQ ID NO: 613 - protein, 15 aa, Artificial sequence
  Peptide
ATATIPLQASLPFGW

SEQ ID NO: 614 - protein, 15 aa, Artificial sequence
  Peptide
TATIPLQASLPFGWL

SEQ ID NO: 615 - protein, 15 aa, Artificial sequence
  Peptide
ATIPLQASLPFGWLV

SEQ ID NO: 616 - protein, 15 aa, Artificial sequence
  Peptide
TIPLQASLPFGWLVI

SEQ ID NO: 617 - protein, 15 aa, Artificial sequence
  Peptide
IPLQASLPFGWLVIG

SEQ ID NO: 618 - protein, 15 aa, Artificial sequence
  Peptide
PLQASLPFGWLVIGV

SEQ ID NO: 619 - protein, 15 aa, Artificial sequence
  Peptide
LQASLPFGWLVIGVA

SEQ ID NO: 620 - protein, 15 aa, Artificial sequence
  Peptide
QASLPFGWLVIGVAF

SEQ ID NO: 621 - protein, 15 aa, Artificial sequence
  Peptide
ASLPFGWLVIGVAFL

SEQ ID NO: 622 - protein, 15 aa, Artificial sequence
  Peptide
SLPFGWLVIGVAFLA

SEQ ID NO: 623 - protein, 15 aa, Artificial sequence
  Peptide
LPFGWLVIGVAFLAV

SEQ ID NO: 624 - protein, 15 aa, Artificial sequence
  Peptide
PFGWLVIGVAFLAVF

SEQ ID NO: 625 - protein, 15 aa, Artificial sequence
  Peptide
FGWLVIGVAFLAVFQ

SEQ ID NO: 626 - protein, 15 aa, Artificial sequence
  Peptide
GWLVIGVAFLAVFQS

SEQ ID NO: 627 - protein, 15 aa, Artificial sequence
  Peptide
WLVIGVAFLAVFQSA

SEQ ID NO: 628 - protein, 15 aa, Artificial sequence
  Peptide
LVIGVAFLAVFQSAT

SEQ ID NO: 629 - protein, 15 aa, Artificial sequence
  Peptide
VIGVAFLAVFQSATK

SEQ ID NO: 630 - protein, 15 aa, Artificial sequence
  Peptide
IGVAFLAVFQSATKI

SEQ ID NO: 631 - protein, 15 aa, Artificial sequence
  Peptide
GVAFLAVFQSATKII

SEQ ID NO: 632 - protein, 15 aa, Artificial sequence
  Peptide
VAFLAVFQSATKIIA

SEQ ID NO: 633 - protein, 15 aa, Artificial sequence
  Peptide
AFLAVFQSATKIIAL

SEQ ID NO: 634 - protein, 15 aa, Artificial sequence
  Peptide
FLAVFQSATKIIALN

SEQ ID NO: 635 - protein, 15 aa, Artificial sequence
  Peptide
LAVFQSATKIIALNK

SEQ ID NO: 636 - protein, 15 aa, Artificial sequence
  Peptide
KRWQLALYKGFQFIC

SEQ ID NO: 637 - protein, 15 aa, Artificial sequence
  Peptide
RWQLALYKGFQFICN

SEQ ID NO: 638 - protein, 15 aa, Artificial sequence
  Peptide
WQLALYKGFQFICNL

SEQ ID NO: 639 - protein, 15 aa, Artificial sequence
  Peptide
QLALYKGFQFICNLL

SEQ ID NO: 640 - protein, 15 aa, Artificial sequence
  Peptide
LALYKGFQFICNLLL

SEQ ID NO: 641 - protein, 15 aa, Artificial sequence
  Peptide
ALYKGFQFICNLLLL

SEQ ID NO: 642 - protein, 15 aa, Artificial sequence
  Peptide
LYKGFQFICNLLLLF

SEQ ID NO: 643 - protein, 15 aa, Artificial sequence
  Peptide
YKGFQFICNLLLLFV

SEQ ID NO: 644 - protein, 15 aa, Artificial sequence
  Peptide
KGFQFICNLLLLFVT

SEQ ID NO: 645 - protein, 15 aa, Artificial sequence
  Peptide
GFQFICNLLLLFVTI

SEQ ID NO: 646 - protein, 15 aa, Artificial sequence
  Peptide
FQFICNLLLLFVTIY

SEQ ID NO: 647 - protein, 15 aa, Artificial sequence
  Peptide
QFICNLLLLFVTIYS

SEQ ID NO: 648 - protein, 15 aa, Artificial sequence
  Peptide
FICNLLLLFVTIYSH

SEQ ID NO: 649 - protein, 15 aa, Artificial sequence
  Peptide
ICNLLLLFVTIYSHL

SEQ ID NO: 650 - protein, 15 aa, Artificial sequence
  Peptide
CNLLLLFVTIYSHLL

SEQ ID NO: 651 - protein, 15 aa, Artificial sequence
  Peptide
NLLLLFVTIYSHLLL

SEQ ID NO: 652 - protein, 15 aa, Artificial sequence
  Peptide
LLLLFVTIYSHLLLV

SEQ ID NO: 653 - protein, 15 aa, Artificial sequence
  Peptide
LLLFVTIYSHLLLVA

SEQ ID NO: 654 - protein, 15 aa, Artificial sequence
  Peptide
LLFVTIYSHLLLVAA

SEQ ID NO: 655 - protein, 15 aa, Artificial sequence
  Peptide
LFVTIYSHLLLVAAG

SEQ ID NO: 656 - protein, 15 aa, Artificial sequence
  Peptide
FVTIYSHLLLVAAGM

SEQ ID NO: 657 - protein, 15 aa, Artificial sequence
  Peptide
VTIYSHLLLVAAGME

SEQ ID NO: 658 - protein, 15 aa, Artificial sequence
  Peptide
TIYSHLLLVAAGMEA

SEQ ID NO: 659 - protein, 15 aa, Artificial sequence
  Peptide
IYSHLLLVAAGMEAQ

SEQ ID NO: 660 - protein, 15 aa, Artificial sequence
  Peptide
YSHLLLVAAGMEAQF

SEQ ID NO: 661 - protein, 15 aa, Artificial sequence
  Peptide
SHLLLVAAGMEAQFL

SEQ ID NO: 662 - protein, 15 aa, Artificial sequence
  Peptide
HLLLVAAGMEAQFLY

SEQ ID NO: 663 - protein, 15 aa, Artificial sequence
  Peptide
LLLVAAGMEAQFLYL

SEQ ID NO: 664 - protein, 15 aa, Artificial sequence
  Peptide
LLVAAGMEAQFLYLY

SEQ ID NO: 665 - protein, 15 aa, Artificial sequence
  Peptide
LVAAGMEAQFLYLYA

SEQ ID NO: 666 - protein, 15 aa, Artificial sequence
  Peptide
VAAGMEAQFLYLYAL

SEQ ID NO: 667 - protein, 15 aa, Artificial sequence
  Peptide
AAGMEAQFLYLYALI

SEQ ID NO: 668 - protein, 15 aa, Artificial sequence
  Peptide
AGMEAQFLYLYALIY

SEQ ID NO: 669 - protein, 15 aa, Artificial sequence
  Peptide
GMEAQFLYLYALIYF

SEQ ID NO: 670 - protein, 15 aa, Artificial sequence
  Peptide
MEAQFLYLYALIYFL

SEQ ID NO: 671 - protein, 15 aa, Artificial sequence
  Peptide
EAQFLYLYALIYFLQ

SEQ ID NO: 672 - protein, 15 aa, Artificial sequence
  Peptide
AQFLYLYALIYFLQC

SEQ ID NO: 673 - protein, 15 aa, Artificial sequence
  Peptide
QFLYLYALIYFLQCI

SEQ ID NO: 674 - protein, 15 aa, Artificial sequence
  Peptide
FLYLYALIYFLQCIN

SEQ ID NO: 675 - protein, 15 aa, Artificial sequence
  Peptide
LYLYALIYFLQCINA

SEQ ID NO: 676 - protein, 15 aa, Artificial sequence
  Peptide
YLYALIYFLQCINAC

SEQ ID NO: 677 - protein, 15 aa, Artificial sequence
  Peptide
LYALIYFLQCINACR

SEQ ID NO: 678 - protein, 15 aa, Artificial sequence
  Peptide
YALIYFLQCINACRI

SEQ ID NO: 679 - protein, 15 aa, Artificial sequence
  Peptide
ALIYFLQCINACRII

SEQ ID NO: 680 - protein, 15 aa, Artificial sequence
  Peptide
LIYFLQCINACRIIM

SEQ ID NO: 681 - protein, 15 aa, Artificial sequence
  Peptide
IYFLQCINACRIIMR

SEQ ID NO: 682 - protein, 15 aa, Artificial sequence
  Peptide
YFLQCINACRIIMRC

SEQ ID NO: 683 - protein, 15 aa, Artificial sequence
  Peptide
FLQCINACRIIMRCW

SEQ ID NO: 684 - protein, 15 aa, Artificial sequence
  Peptide
WKCKSKNPLLYDANY

SEQ ID NO: 685 - protein, 15 aa, Artificial sequence
  Peptide
KCKSKNPLLYDANYF

SEQ ID NO: 686 - protein, 15 aa, Artificial sequence
  Peptide
CKSKNPLLYDANYFV

SEQ ID NO: 687 - protein, 15 aa, Artificial sequence
  Peptide
KSKNPLLYDANYFVC

SEQ ID NO: 688 - protein, 15 aa, Artificial sequence
  Peptide
SKNPLLYDANYFVCW

SEQ ID NO: 689 - protein, 15 aa, Artificial sequence
  Peptide
KNPLLYDANYFVCWH

SEQ ID NO: 690 - protein, 15 aa, Artificial sequence
  Peptide
NPLLYDANYFVCWHT

SEQ ID NO: 691 - protein, 15 aa, Artificial sequence
  Peptide
PLLYDANYFVCWHTH

SEQ ID NO: 692 - protein, 15 aa, Artificial sequence
  Peptide
LLYDANYFVCWHTHN

SEQ ID NO: 693 - protein, 15 aa, Artificial sequence
  Peptide
LYDANYFVCWHTHNY

SEQ ID NO: 694 - protein, 15 aa, Artificial sequence
  Peptide
CIPYNSVTDTIVVTE

SEQ ID NO: 695 - protein, 15 aa, Artificial sequence
  Peptide
IPYNSVTDTIVVTEG

SEQ ID NO: 696 - protein, 15 aa, Artificial sequence
  Peptide
PYNSVTDTIVVTEGD

SEQ ID NO: 697 - protein, 15 aa, Artificial sequence
  Peptide
YNSVTDTIVVTEGDG

SEQ ID NO: 698 - protein, 15 aa, Artificial sequence
  Peptide
NSVTDTIVVTEGDGI

SEQ ID NO: 699 - protein, 15 aa, Artificial sequence
  Peptide
SVTDTIVVTEGDGIS

SEQ ID NO: 700 - protein, 15 aa, Artificial sequence
  Peptide
VTDTIVVTEGDGIST

SEQ ID NO: 701 - protein, 15 aa, Artificial sequence
  Peptide
TDTIVVTEGDGISTP

SEQ ID NO: 702 - protein, 15 aa, Artificial sequence
  Peptide
DTIVVTEGDGISTPK

SEQ ID NO: 703 - protein, 15 aa, Artificial sequence
  Peptide
TIVVTEGDGISTPKL

SEQ ID NO: 704 - protein, 15 aa, Artificial sequence
  Peptide
IVVTEGDGISTPKLK

SEQ ID NO: 705 - protein, 15 aa, Artificial sequence
  Peptide
VVTEGDGISTPKLKE

SEQ ID NO: 706 - protein, 15 aa, Artificial sequence
  Peptide
VTEGDGISTPKLKED

SEQ ID NO: 707 - protein, 15 aa, Artificial sequence
  Peptide
TEGDGISTPKLKEDY

SEQ ID NO: 708 - protein, 15 aa, Artificial sequence
  Peptide
EGDGISTPKLKEDYQ

SEQ ID NO: 709 - protein, 15 aa, Artificial sequence
  Peptide
VKDYVVVHGYFTEVY

SEQ ID NO: 710 - protein, 15 aa, Artificial sequence
  Peptide
KDYVVVHGYFTEVYY

SEQ ID NO: 711 - protein, 15 aa, Artificial sequence
  Peptide
DYVVVHGYFTEVYYQ

SEQ ID NO: 712 - protein, 15 aa, Artificial sequence
  Peptide
YVVVHGYFTEVYYQL

SEQ ID NO: 713 - protein, 15 aa, Artificial sequence
  Peptide
VVVHGYFTEVYYQLE

SEQ ID NO: 714 - protein, 15 aa, Artificial sequence
  Peptide
VVHGYFTEVYYQLES

SEQ ID NO: 715 - protein, 15 aa, Artificial sequence
  Peptide
VHGYFTEVYYQLEST

SEQ ID NO: 716 - protein, 15 aa, Artificial sequence
  Peptide
HGYFTEVYYQLESTQ

SEQ ID NO: 717 - protein, 15 aa, Artificial sequence
  Peptide
GYFTEVYYQLESTQI

SEQ ID NO: 718 - protein, 15 aa, Artificial sequence
  Peptide
YFTEVYYQLESTQIT

SEQ ID NO: 719 - protein, 15 aa, Artificial sequence
  Peptide
FTEVYYQLESTQITT

SEQ ID NO: 720 - protein, 15 aa, Artificial sequence
  Peptide
TEVYYQLESTQITTD

SEQ ID NO: 721 - protein, 15 aa, Artificial sequence
  Peptide
EVYYQLESTQITTDT

SEQ ID NO: 722 - protein, 15 aa, Artificial sequence
  Peptide
VYYQLESTQITTDTG

SEQ ID NO: 723 - protein, 15 aa, Artificial sequence
  Peptide
YYQLESTQITTDTGI

SEQ ID NO: 724 - protein, 15 aa, Artificial sequence
  Peptide
YQLESTQITTDTGIE

SEQ ID NO: 725 - protein, 15 aa, Artificial sequence
  Peptide
QLESTQITTDTGIEN

SEQ ID NO: 726 - protein, 15 aa, Artificial sequence
  Peptide
LESTQITTDTGIENA

SEQ ID NO: 727 - protein, 15 aa, Artificial sequence
  Peptide
ESTQITTDTGIENAT

SEQ ID NO: 728 - protein, 15 aa, Artificial sequence
  Peptide
STQITTDTGIENATF

SEQ ID NO: 729 - protein, 15 aa, Artificial sequence
  Peptide
TQITTDTGIENATFF

SEQ ID NO: 730 - protein, 15 aa, Artificial sequence
  Peptide
QITTDTGIENATFFI

SEQ ID NO: 731 - protein, 15 aa, Artificial sequence
  Peptide
ITTDTGIENATFFIF

SEQ ID NO: 732 - protein, 15 aa, Artificial sequence
  Peptide
TTDTGIENATFFIFN

SEQ ID NO: 733 - protein, 15 aa, Artificial sequence
  Peptide
TDTGIENATFFIFNK

SEQ ID NO: 734 - protein, 15 aa, Artificial sequence
  Peptide
DTGIENATFFIFNKL

SEQ ID NO: 735 - protein, 15 aa, Artificial sequence
  Peptide
TGIENATFFIFNKLV

SEQ ID NO: 736 - protein, 15 aa, Artificial sequence
  Peptide
GIENATFFIFNKLVK

SEQ ID NO: 737 - protein, 15 aa, Artificial sequence
  Peptide
IENATFFIFNKLVKD

SEQ ID NO: 738 - protein, 15 aa, Artificial sequence
  Peptide
ENATFFIFNKLVKDP

SEQ ID NO: 739 - protein, 15 aa, Artificial sequence
  Peptide
NATFFIFNKLVKDPP

SEQ ID NO: 740 - protein, 15 aa, Artificial sequence
  Peptide
DPPNVQIHTIDGSSG

SEQ ID NO: 741 - protein, 15 aa, Artificial sequence
  Peptide
PPNVQIHTIDGSSGV

SEQ ID NO: 742 - protein, 15 aa, Artificial sequence
  Peptide
PNVQIHTIDGSSGVA

SEQ ID NO: 743 - protein, 15 aa, Artificial sequence
  Peptide
NVQIHTIDGSSGVAN

SEQ ID NO: 744 - protein, 15 aa, Artificial sequence
  Peptide
VQIHTIDGSSGVANP

SEQ ID NO: 745 - protein, 15 aa, Artificial sequence
  Peptide
QIHTIDGSSGVANPA

SEQ ID NO: 746 - protein, 15 aa, Artificial sequence
  Peptide
IHTIDGSSGVANPAM

SEQ ID NO: 747 - protein, 15 aa, Artificial sequence
  Peptide
HTIDGSSGVANPAMD

SEQ ID NO: 748 - protein, 15 aa, Artificial sequence
  Peptide
TIDGSSGVANPAMDP

SEQ ID NO: 749 - protein, 15 aa, Artificial sequence
  Peptide
IDGSSGVANPAMDPI

SEQ ID NO: 750 - protein, 15 aa, Artificial sequence
  Peptide
THITMTTVYHITVSQ

SEQ ID NO: 751 - protein, 15 aa, Artificial sequence
  Peptide
HITMTTVYHITVSQI

SEQ ID NO: 752 - protein, 15 aa, Artificial sequence
  Peptide
ITMTTVYHITVSQIQ

SEQ ID NO: 753 - protein, 15 aa, Artificial sequence
  Peptide
TMTTVYHITVSQIQL

SEQ ID NO: 754 - protein, 15 aa, Artificial sequence
  Peptide
MTTVYHITVSQIQLS

SEQ ID NO: 755 - protein, 15 aa, Artificial sequence
  Peptide
TTVYHITVSQIQLSL

SEQ ID NO: 756 - protein, 15 aa, Artificial sequence
  Peptide
TVYHITVSQIQLSLL

SEQ ID NO: 757 - protein, 15 aa, Artificial sequence
  Peptide
VYHITVSQIQLSLLK

SEQ ID NO: 758 - protein, 15 aa, Artificial sequence
  Peptide
YHITVSQIQLSLLKV

SEQ ID NO: 759 - protein, 15 aa, Artificial sequence
  Peptide
HITVSQIQLSLLKVT

SEQ ID NO: 760 - protein, 15 aa, Artificial sequence
  Peptide
ITVSQIQLSLLKVTA

SEQ ID NO: 761 - protein, 15 aa, Artificial sequence
  Peptide
TVSQIQLSLLKVTAF

SEQ ID NO: 762 - protein, 15 aa, Artificial sequence
  Peptide
VSQIQLSLLKVTAFQ

SEQ ID NO: 763 - protein, 15 aa, Artificial sequence
  Peptide
SQIQLSLLKVTAFQH

SEQ ID NO: 764 - protein, 15 aa, Artificial sequence
  Peptide
QIQLSLLKVTAFQHQ

SEQ ID NO: 765 - protein, 15 aa, Artificial sequence
  Peptide
IQLSLLKVTAFQHQN

SEQ ID NO: 766 - protein, 15 aa, Artificial sequence
  Peptide
QLSLLKVTAFQHQNS

SEQ ID NO: 767 - protein, 15 aa, Artificial sequence
  Peptide
LSLLKVTAFQHQNSK

SEQ ID NO: 768 - protein, 15 aa, Artificial sequence
  Peptide
SLLKVTAFQHQNSKK

SEQ ID NO: 769 - protein, 15 aa, Artificial sequence
  Peptide
LLKVTAFQHQNSKKT

SEQ ID NO: 770 - protein, 15 aa, Artificial sequence
  Peptide
LKVTAFQHQNSKKTT

SEQ ID NO: 771 - protein, 15 aa, Artificial sequence
  Peptide
KVTAFQHQNSKKTTK

SEQ ID NO: 772 - protein, 15 aa, Artificial sequence
  Peptide
VTAFQHQNSKKTTKL

SEQ ID NO: 773 - protein, 15 aa, Artificial sequence
  Peptide
LVVILRIGTQVLKTM

SEQ ID NO: 774 - protein, 15 aa, Artificial sequence
  Peptide
VVILRIGTQVLKTMS

SEQ ID NO: 775 - protein, 15 aa, Artificial sequence
  Peptide
VILRIGTQVLKTMSL

SEQ ID NO: 776 - protein, 15 aa, Artificial sequence
  Peptide
ILRIGTQVLKTMSLY

SEQ ID NO: 777 - protein, 15 aa, Artificial sequence
  Peptide
TMSLYMAISPKFTTS

SEQ ID NO: 778 - protein, 15 aa, Artificial sequence
  Peptide
MSLYMAISPKFTTSL

SEQ ID NO: 779 - protein, 15 aa, Artificial sequence
  Peptide
SLYMAISPKFTTSLS

SEQ ID NO: 780 - protein, 15 aa, Artificial sequence
  Peptide
LYMAISPKFTTSLSL

SEQ ID NO: 781 - protein, 15 aa, Artificial sequence
  Peptide
YMAISPKFTTSLSLH

SEQ ID NO: 782 - protein, 15 aa, Artificial sequence
  Peptide
MAISPKFTTSLSLHK

SEQ ID NO: 783 - protein, 15 aa, Artificial sequence
  Peptide
AISPKFTTSLSLHKL

SEQ ID NO: 784 - protein, 15 aa, Artificial sequence
  Peptide
ISPKFTTSLSLHKLL

SEQ ID NO: 785 - protein, 15 aa, Artificial sequence
  Peptide
SPKFTTSLSLHKLLQ

SEQ ID NO: 786 - protein, 15 aa, Artificial sequence
  Peptide
PKFTTSLSLHKLLQT

SEQ ID NO: 787 - protein, 15 aa, Artificial sequence
  Peptide
KFTTSLSLHKLLQTL

SEQ ID NO: 788 - protein, 15 aa, Artificial sequence
  Peptide
FTTSLSLHKLLQTLV

SEQ ID NO: 789 - protein, 15 aa, Artificial sequence
  Peptide
TTSLSLHKLLQTLVL

SEQ ID NO: 790 - protein, 15 aa, Artificial sequence
  Peptide
TSLSLHKLLQTLVLK

SEQ ID NO: 791 - protein, 15 aa, Artificial sequence
  Peptide
SLSLHKLLQTLVLKM

SEQ ID NO: 792 - protein, 15 aa, Artificial sequence
  Peptide
LSLHKLLQTLVLKML

SEQ ID NO: 793 - protein, 15 aa, Artificial sequence
  Peptide
SLHKLLQTLVLKMLH

SEQ ID NO: 794 - protein, 15 aa, Artificial sequence
  Peptide
LHKLLQTLVLKMLHS

SEQ ID NO: 795 - protein, 15 aa, Artificial sequence
  Peptide
HKLLQTLVLKMLHSS

SEQ ID NO: 796 - protein, 15 aa, Artificial sequence
  Peptide
KLLQTLVLKMLHSSS

SEQ ID NO: 797 - protein, 15 aa, Artificial sequence
  Peptide
LLQTLVLKMLHSSSL

SEQ ID NO: 798 - protein, 15 aa, Artificial sequence
  Peptide
LQTLVLKMLHSSSLT

SEQ ID NO: 799 - protein, 15 aa, Artificial sequence
  Peptide
QTLVLKMLHSSSLTS

SEQ ID NO: 800 - protein, 15 aa, Artificial sequence
  Peptide
TLVLKMLHSSSLTSL

SEQ ID NO: 801 - protein, 15 aa, Artificial sequence
  Peptide
LVLKMLHSSSLTSLL

SEQ ID NO: 802 - protein, 15 aa, Artificial sequence
  Peptide
VLKMLHSSSLTSLLK

SEQ ID NO: 803 - protein, 15 aa, Artificial sequence
  Peptide
LKMLHSSSLTSLLKT

SEQ ID NO: 804 - protein, 15 aa, Artificial sequence
  Peptide
KMLHSSSLTSLLKTH

SEQ ID NO: 805 - protein, 15 aa, Artificial sequence
  Peptide
MLHSSSLTSLLKTHR

SEQ ID NO: 806 - protein, 15 aa, Artificial sequence
  Peptide
LHSSSLTSLLKTHRM

SEQ ID NO: 807 - protein, 15 aa, Artificial sequence
  Peptide
HSSSLTSLLKTHRMC

SEQ ID NO: 808 - protein, 15 aa, Artificial sequence
  Peptide
SSSLTSLLKTHRMCK

SEQ ID NO: 809 - protein, 15 aa, Artificial sequence
  Peptide
SSLTSLLKTHRMCKY

SEQ ID NO: 810 - protein, 15 aa, Artificial sequence
  Peptide
SLTSLLKTHRMCKYT

SEQ ID NO: 811 - protein, 15 aa, Artificial sequence
  Peptide
LTSLLKTHRMCKYTQ

SEQ ID NO: 812 - protein, 15 aa, Artificial sequence
  Peptide
TSLLKTHRMCKYTQS

SEQ ID NO: 813 - protein, 15 aa, Artificial sequence
  Peptide
SLLKTHRMCKYTQST

SEQ ID NO: 814 - protein, 15 aa, Artificial sequence
  Peptide
LLKTHRMCKYTQSTA

SEQ ID NO: 815 - protein, 15 aa, Artificial sequence
  Peptide
LKTHRMCKYTQSTAL

SEQ ID NO: 816 - protein, 15 aa, Artificial sequence
  Peptide
KTHRMCKYTQSTALQ

SEQ ID NO: 817 - protein, 15 aa, Artificial sequence
  Peptide
THRMCKYTQSTALQE

SEQ ID NO: 818 - protein, 15 aa, Artificial sequence
  Peptide
HRMCKYTQSTALQEL

SEQ ID NO: 819 - protein, 15 aa, Artificial sequence
  Peptide
RMCKYTQSTALQELL

SEQ ID NO: 820 - protein, 15 aa, Artificial sequence
  Peptide
MCKYTQSTALQELLI

SEQ ID NO: 821 - protein, 15 aa, Artificial sequence
  Peptide
CKYTQSTALQELLIQ

SEQ ID NO: 822 - protein, 15 aa, Artificial sequence
  Peptide
KYTQSTALQELLIQQ

SEQ ID NO: 823 - protein, 15 aa, Artificial sequence
  Peptide
YTQSTALQELLIQQW

SEQ ID NO: 824 - protein, 15 aa, Artificial sequence
  Peptide
TQSTALQELLIQQWI

SEQ ID NO: 825 - protein, 15 aa, Artificial sequence
  Peptide
QSTALQELLIQQWIQ

SEQ ID NO: 826 - protein, 15 aa, Artificial sequence
  Peptide
STALQELLIQQWIQF

SEQ ID NO: 827 - protein, 15 aa, Artificial sequence
  Peptide
TALQELLIQQWIQFM

SEQ ID NO: 828 - protein, 15 aa, Artificial sequence
  Peptide
ALQELLIQQWIQFMM

SEQ ID NO: 829 - protein, 15 aa, Artificial sequence
  Peptide
LQELLIQQWIQFMMS

SEQ ID NO: 830 - protein, 15 aa, Artificial sequence
  Peptide
QELLIQQWIQFMMSR

SEQ ID NO: 831 - protein, 15 aa, Artificial sequence
  Peptide
ELLIQQWIQFMMSRR

SEQ ID NO: 832 - protein, 15 aa, Artificial sequence
  Peptide
LLIQQWIQFMMSRRR

SEQ ID NO: 833 - protein, 15 aa, Artificial sequence
  Peptide
LIQQWIQFMMSRRRL

SEQ ID NO: 834 - protein, 15 aa, Artificial sequence
  Peptide
IQQWIQFMMSRRRLL

SEQ ID NO: 835 - protein, 15 aa, Artificial sequence
  Peptide
QQWIQFMMSRRRLLA

SEQ ID NO: 836 - protein, 15 aa, Artificial sequence
  Peptide
QWIQFMMSRRRLLAC

SEQ ID NO: 837 - protein, 15 aa, Artificial sequence
  Peptide
WIQFMMSRRRLLACL

SEQ ID NO: 838 - protein, 15 aa, Artificial sequence
  Peptide
IQFMMSRRRLLACLC

SEQ ID NO: 839 - protein, 15 aa, Artificial sequence
  Peptide
QFMMSRRRLLACLCK

SEQ ID NO: 840 - protein, 15 aa, Artificial sequence
  Peptide
FMMSRRRLLACLCKH

SEQ ID NO: 841 - protein, 15 aa, Artificial sequence
  Peptide
MYSFVSEETGTLIVN

SEQ ID NO: 842 - protein, 15 aa, Artificial sequence
  Peptide
YSFVSEETGTLIVNS

SEQ ID NO: 843 - protein, 15 aa, Artificial sequence
  Peptide
SFVSEETGTLIVNSV

SEQ ID NO: 844 - protein, 15 aa, Artificial sequence
  Peptide
VSEETGTLIVNSVLL

SEQ ID NO: 845 - protein, 15 aa, Artificial sequence
  Peptide
FVSEETGTLIVNSVL

SEQ ID NO: 846 - protein, 15 aa, Artificial sequence
  Peptide
SEETGTLIVNSVLLF

SEQ ID NO: 847 - protein, 15 aa, Artificial sequence
  Peptide
EETGTLIVNSVLLFL

SEQ ID NO: 848 - protein, 15 aa, Artificial sequence
  Peptide
ETGTLIVNSVLLFLA

SEQ ID NO: 849 - protein, 15 aa, Artificial sequence
  Peptide
TGTLIVNSVLLFLAF

SEQ ID NO: 850 - protein, 15 aa, Artificial sequence
  Peptide
GTLIVNSVLLFLAFV

SEQ ID NO: 851 - protein, 15 aa, Artificial sequence
  Peptide
TLIVNSVLLFLAFVV

SEQ ID NO: 852 - protein, 15 aa, Artificial sequence
  Peptide
LIVNSVLLFLAFVVF

SEQ ID NO: 853 - protein, 15 aa, Artificial sequence
  Peptide
IVNSVLLFLAFVVFL

SEQ ID NO: 854 - protein, 15 aa, Artificial sequence
  Peptide
VNSVLLFLAFVVFLL

SEQ ID NO: 855 - protein, 15 aa, Artificial sequence
  Peptide
NSVLLFLAFVVFLLV

SEQ ID NO: 856 - protein, 15 aa, Artificial sequence
  Peptide
SVLLFLAFVVFLLVT

SEQ ID NO: 857 - protein, 15 aa, Artificial sequence
  Peptide
VLLFLAFVVFLLVTL

SEQ ID NO: 858 - protein, 15 aa, Artificial sequence
  Peptide
LLFLAFVVFLLVTLA

SEQ ID NO: 859 - protein, 15 aa, Artificial sequence
  Peptide
LFLAFVVFLLVTLAI

SEQ ID NO: 860 - protein, 15 aa, Artificial sequence
  Peptide
FLAFVVFLLVTLAIL

SEQ ID NO: 861 - protein, 15 aa, Artificial sequence
  Peptide
LAFVVFLLVTLAILT

SEQ ID NO: 862 - protein, 15 aa, Artificial sequence
  Peptide
AFVVFLLVTLAILTA

SEQ ID NO: 863 - protein, 15 aa, Artificial sequence
  Peptide
FVVFLLVTLAILTAL

SEQ ID NO: 864 - protein, 15 aa, Artificial sequence
  Peptide
VVFLLVTLAILTALR

SEQ ID NO: 865 - protein, 15 aa, Artificial sequence
  Peptide
VFLLVTLAILTALRL

SEQ ID NO: 866 - protein, 15 aa, Artificial sequence
  Peptide
FLLVTLAILTALRLC

SEQ ID NO: 867 - protein, 15 aa, Artificial sequence
  Peptide
LLVTLAILTALRLCA

SEQ ID NO: 868 - protein, 15 aa, Artificial sequence
  Peptide
LVTLAILTALRLCAY

SEQ ID NO: 869 - protein, 15 aa, Artificial sequence
  Peptide
VTLAILTALRLCAYC

SEQ ID NO: 870 - protein, 15 aa, Artificial sequence
  Peptide
TLAILTALRLCAYCC

SEQ ID NO: 871 - protein, 15 aa, Artificial sequence
  Peptide
LAILTALRLCAYCCN

SEQ ID NO: 872 - protein, 15 aa, Artificial sequence
  Peptide
AILTALRLCAYCCNI

SEQ ID NO: 873 - protein, 15 aa, Artificial sequence
  Peptide
ILTALRLCAYCCNIV

SEQ ID NO: 874 - protein, 15 aa, Artificial sequence
  Peptide
LTALRLCAYCCNIVN

SEQ ID NO: 875 - protein, 15 aa, Artificial sequence
  Peptide
TALRLCAYCCNIVNV

SEQ ID NO: 876 - protein, 15 aa, Artificial sequence
  Peptide
ALRLCAYCCNIVNVS

SEQ ID NO: 877 - protein, 15 aa, Artificial sequence
  Peptide
LRLCAYCCNIVNVSL

SEQ ID NO: 878 - protein, 15 aa, Artificial sequence
  Peptide
RLCAYCCNIVNVSLV

SEQ ID NO: 879 - protein, 15 aa, Artificial sequence
  Peptide
ELKQLLEQWNLVIGF

SEQ ID NO: 880 - protein, 15 aa, Artificial sequence
  Peptide
LKQLLEQWNLVIGFL

SEQ ID NO: 881 - protein, 15 aa, Artificial sequence
  Peptide
KQLLEQWNLVIGFLF

SEQ ID NO: 882 - protein, 15 aa, Artificial sequence
  Peptide
QLLEQWNLVIGFLFL

SEQ ID NO: 883 - protein, 15 aa, Artificial sequence
  Peptide
LLEQWNLVIGFLFLA

SEQ ID NO: 884 - protein, 15 aa, Artificial sequence
  Peptide
LEQWNLVIGFLFLAW

SEQ ID NO: 885 - protein, 15 aa, Artificial sequence
  Peptide
EQWNLVIGFLFLAWI

SEQ ID NO: 886 - protein, 15 aa, Artificial sequence
  Peptide
QWNLVIGFLFLAWIM

SEQ ID NO: 887 - protein, 15 aa, Artificial sequence
  Peptide
WNLVIGFLFLAWIML

SEQ ID NO: 888 - protein, 15 aa, Artificial sequence
  Peptide
NLVIGFLFLAWIMLL

SEQ ID NO: 889 - protein, 15 aa, Artificial sequence
  Peptide
LVIGFLFLAWIMLLQ

SEQ ID NO: 890 - protein, 15 aa, Artificial sequence
  Peptide
VIGFLFLAWIMLLQF

SEQ ID NO: 891 - protein, 15 aa, Artificial sequence
  Peptide
IGFLFLAWIMLLQFA

SEQ ID NO: 892 - protein, 15 aa, Artificial sequence
  Peptide
GFLFLAWIMLLQFAY

SEQ ID NO: 893 - protein, 15 aa, Artificial sequence
  Peptide
FLFLAWIMLLQFAYS

SEQ ID NO: 894 - protein, 15 aa, Artificial sequence
  Peptide
LFLAWIMLLQFAYSN

SEQ ID NO: 895 - protein, 15 aa, Artificial sequence
  Peptide
FLAWIMLLQFAYSNR

SEQ ID NO: 896 - protein, 15 aa, Artificial sequence
  Peptide
LAWIMLLQFAYSNRN

SEQ ID NO: 897 - protein, 15 aa, Artificial sequence
  Peptide
AWIMLLQFAYSNRNR

SEQ ID NO: 898 - protein, 15 aa, Artificial sequence
  Peptide
WIMLLQFAYSNRNRF

SEQ ID NO: 899 - protein, 15 aa, Artificial sequence
  Peptide
IMLLQFAYSNRNRFL

SEQ ID NO: 900 - protein, 15 aa, Artificial sequence
  Peptide
MLLQFAYSNRNRFLY

SEQ ID NO: 901 - protein, 15 aa, Artificial sequence
  Peptide
LLQFAYSNRNRFLYI

SEQ ID NO: 902 - protein, 15 aa, Artificial sequence
  Peptide
LQFAYSNRNRFLYII

SEQ ID NO: 903 - protein, 15 aa, Artificial sequence
  Peptide
LYIIKLVFLWLLWPV

SEQ ID NO: 904 - protein, 15 aa, Artificial sequence
  Peptide
YIIKLVFLWLLWPVT

SEQ ID NO: 905 - protein, 15 aa, Artificial sequence
  Peptide
IIKLVFLWLLWPVTL

SEQ ID NO: 906 - protein, 15 aa, Artificial sequence
  Peptide
IKLVFLWLLWPVTLA

SEQ ID NO: 907 - protein, 15 aa, Artificial sequence
  Peptide
KLVFLWLLWPVTLAC

SEQ ID NO: 908 - protein, 15 aa, Artificial sequence
  Peptide
LVFLWLLWPVTLACF

SEQ ID NO: 909 - protein, 15 aa, Artificial sequence
  Peptide
VFLWLLWPVTLACFV

SEQ ID NO: 910 - protein, 15 aa, Artificial sequence
  Peptide
FLWLLWPVTLACFVL

SEQ ID NO: 911 - protein, 15 aa, Artificial sequence
  Peptide
LWLLWPVTLACFVLA

SEQ ID NO: 912 - protein, 15 aa, Artificial sequence
  Peptide
WLLWPVTLACFVLAA

SEQ ID NO: 913 - protein, 15 aa, Artificial sequence
  Peptide
LLWPVTLACFVLAAV

SEQ ID NO: 914 - protein, 15 aa, Artificial sequence
  Peptide
LWPVTLACFVLAAVY

SEQ ID NO: 915 - protein, 15 aa, Artificial sequence
  Peptide
WPVTLACFVLAAVYR

SEQ ID NO: 916 - protein, 15 aa, Artificial sequence
  Peptide
PVTLACFVLAAVYRI

SEQ ID NO: 917 - protein, 15 aa, Artificial sequence
  Peptide
VTLACFVLAAVYRIN

SEQ ID NO: 918 - protein, 15 aa, Artificial sequence
  Peptide
TLACFVLAAVYRINW

SEQ ID NO: 919 - protein, 15 aa, Artificial sequence
  Peptide
LACFVLAAVYRINWV

SEQ ID NO: 920 - protein, 15 aa, Artificial sequence
  Peptide
ACFVLAAVYRINWVT

SEQ ID NO: 921 - protein, 15 aa, Artificial sequence
  Peptide
CFVLAAVYRINWVTG

SEQ ID NO: 922 - protein, 15 aa, Artificial sequence
  Peptide
FVLAAVYRINWVTGG

SEQ ID NO: 923 - protein, 15 aa, Artificial sequence
  Peptide
VLAAVYRINWVTGGI

SEQ ID NO: 924 - protein, 15 aa, Artificial sequence
  Peptide
LAAVYRINWVTGGIA

SEQ ID NO: 925 - protein, 15 aa, Artificial sequence
  Peptide
AAVYRINWVTGGIAI

SEQ ID NO: 926 - protein, 15 aa, Artificial sequence
  Peptide
AVYRINWVTGGIAIA

SEQ ID NO: 927 - protein, 15 aa, Artificial sequence
  Peptide
VYRINWVTGGIAIAM

SEQ ID NO: 928 - protein, 15 aa, Artificial sequence
  Peptide
YRINWVTGGIAIAMA

SEQ ID NO: 929 - protein, 15 aa, Artificial sequence
  Peptide
RINWVTGGIAIAMAC

SEQ ID NO: 930 - protein, 15 aa, Artificial sequence
  Peptide
AIAMACIVGLMWLSY

SEQ ID NO: 931 - protein, 15 aa, Artificial sequence
  Peptide
IAMACIVGLMWLSYF

SEQ ID NO: 932 - protein, 15 aa, Artificial sequence
  Peptide
AMACIVGLMWLSYFV

SEQ ID NO: 933 - protein, 15 aa, Artificial sequence
  Peptide
MACIVGLMWLSYFVA

SEQ ID NO: 934 - protein, 15 aa, Artificial sequence
  Peptide
ACIVGLMWLSYFVAS

SEQ ID NO: 935 - protein, 15 aa, Artificial sequence
  Peptide
CIVGLMWLSYFVASF

SEQ ID NO: 936 - protein, 15 aa, Artificial sequence
  Peptide
IVGLMWLSYFVASFR

SEQ ID NO: 937 - protein, 15 aa, Artificial sequence
  Peptide
VGLMWLSYFVASFRL

SEQ ID NO: 938 - protein, 15 aa, Artificial sequence
  Peptide
GLMWLSYFVASFRLF

SEQ ID NO: 939 - protein, 15 aa, Artificial sequence
  Peptide
ASFRLFARTRSMWSF

SEQ ID NO: 940 - protein, 15 aa, Artificial sequence
  Peptide
SFRLFARTRSMWSFN

SEQ ID NO: 941 - protein, 15 aa, Artificial sequence
  Peptide
FRLFARTRSMWSFNP

SEQ ID NO: 942 - protein, 15 aa, Artificial sequence
  Peptide
RLFARTRSMWSFNPE

SEQ ID NO: 943 - protein, 15 aa, Artificial sequence
  Peptide
LFARTRSMWSFNPET

SEQ ID NO: 944 - protein, 15 aa, Artificial sequence
  Peptide
FARTRSMWSFNPETN

SEQ ID NO: 945 - protein, 15 aa, Artificial sequence
  Peptide
ARTRSMWSFNPETNI

SEQ ID NO: 946 - protein, 15 aa, Artificial sequence
  Peptide
RTRSMWSFNPETNIL

SEQ ID NO: 947 - protein, 15 aa, Artificial sequence
  Peptide
TRSMWSFNPETNILL

SEQ ID NO: 948 - protein, 15 aa, Artificial sequence
  Peptide
RSMWSFNPETNILLN

SEQ ID NO: 949 - protein, 15 aa, Artificial sequence
  Peptide
SMWSFNPETNILLNV

SEQ ID NO: 950 - protein, 15 aa, Artificial sequence
  Peptide
MWSFNPETNILLNVP

SEQ ID NO: 951 - protein, 15 aa, Artificial sequence
  Peptide
WSFNPETNILLNVPL

SEQ ID NO: 952 - protein, 15 aa, Artificial sequence
  Peptide
SFNPETNILLNVPLR

SEQ ID NO: 953 - protein, 15 aa, Artificial sequence
  Peptide
FNPETNILLNVPLRG

SEQ ID NO: 954 - protein, 15 aa, Artificial sequence
  Peptide
NPETNILLNVPLRGT

SEQ ID NO: 955 - protein, 15 aa, Artificial sequence
  Peptide
PETNILLNVPLRGTI

SEQ ID NO: 956 - protein, 15 aa, Artificial sequence
  Peptide
ETNILLNVPLRGTIV

SEQ ID NO: 957 - protein, 15 aa, Artificial sequence
  Peptide
TNILLNVPLRGTIVT

SEQ ID NO: 958 - protein, 15 aa, Artificial sequence
  Peptide
PLMESELVIGAVIIR

SEQ ID NO: 959 - protein, 15 aa, Artificial sequence
  Peptide
LMESELVIGAVIIRG

SEQ ID NO: 960 - protein, 15 aa, Artificial sequence
  Peptide
MESELVIGAVIIRGH

SEQ ID NO: 961 - protein, 15 aa, Artificial sequence
  Peptide
ESELVIGAVIIRGHL

SEQ ID NO: 962 - protein, 15 aa, Artificial sequence
  Peptide
SELVIGAVIIRGHLR

SEQ ID NO: 963 - protein, 15 aa, Artificial sequence
  Peptide
ELVIGAVIIRGHLRM

SEQ ID NO: 964 - protein, 15 aa, Artificial sequence
  Peptide
LVIGAVIIRGHLRMA

SEQ ID NO: 965 - protein, 15 aa, Artificial sequence
  Peptide
RCDIKDLPKEITVAT

SEQ ID NO: 966 - protein, 15 aa, Artificial sequence
  Peptide
CDIKDLPKEITVATS

SEQ ID NO: 967 - protein, 15 aa, Artificial sequence
  Peptide
DIKDLPKEITVATSR

SEQ ID NO: 968 - protein, 15 aa, Artificial sequence
  Peptide
IKDLPKEITVATSRT

SEQ ID NO: 969 - protein, 15 aa, Artificial sequence
  Peptide
KDLPKEITVATSRTL

SEQ ID NO: 970 - protein, 15 aa, Artificial sequence
  Peptide
DLPKEITVATSRTLS

SEQ ID NO: 971 - protein, 15 aa, Artificial sequence
  Peptide
LPKEITVATSRTLSY

SEQ ID NO: 972 - protein, 15 aa, Artificial sequence
  Peptide
PKEITVATSRTLSYY

SEQ ID NO: 973 - protein, 15 aa, Artificial sequence
  Peptide
KEITVATSRTLSYYK

SEQ ID NO: 974 - protein, 15 aa, Artificial sequence
  Peptide
EITVATSRTLSYYKL

SEQ ID NO: 975 - protein, 15 aa, Artificial sequence
  Peptide
ITVATSRTLSYYKLG

SEQ ID NO: 976 - protein, 15 aa, Artificial sequence
  Peptide
TVATSRTLSYYKLGA

SEQ ID NO: 977 - protein, 15 aa, Artificial sequence
  Peptide
VATSRTLSYYKLGAS

SEQ ID NO: 978 - protein, 15 aa, Artificial sequence
  Peptide
ATSRTLSYYKLGASQ

SEQ ID NO: 979 - protein, 15 aa, Artificial sequence
  Peptide
TSRTLSYYKLGASQR

SEQ ID NO: 980 - protein, 15 aa, Artificial sequence
  Peptide
SRTLSYYKLGASQRV

SEQ ID NO: 981 - protein, 15 aa, Artificial sequence
  Peptide
RTLSYYKLGASQRVG

SEQ ID NO: 982 - protein, 15 aa, Artificial sequence
  Peptide
SQRVGTDSGFAAYNR

SEQ ID NO: 983 - protein, 15 aa, Artificial sequence
  Peptide
QRVGTDSGFAAYNRY

SEQ ID NO: 984 - protein, 15 aa, Artificial sequence
  Peptide
RVGTDSGFAAYNRYR

SEQ ID NO: 985 - protein, 15 aa, Artificial sequence
  Peptide
VGTDSGFAAYNRYRI

SEQ ID NO: 986 - protein, 15 aa, Artificial sequence
  Peptide
GTDSGFAAYNRYRIG

SEQ ID NO: 987 - protein, 15 aa, Artificial sequence
  Peptide
TDSGFAAYNRYRIGN

SEQ ID NO: 988 - protein, 15 aa, Artificial sequence
  Peptide
DSGFAAYNRYRIGNY

SEQ ID NO: 989 - protein, 15 aa, Artificial sequence
  Peptide
SGFAAYNRYRIGNYK

SEQ ID NO: 990 - protein, 15 aa, Artificial sequence
  Peptide
GFAAYNRYRIGNYKL

SEQ ID NO: 991 - protein, 15 aa, Artificial sequence
  Peptide
FAAYNRYRIGNYKLN

SEQ ID NO: 992 - protein, 15 aa, Artificial sequence
  Peptide
AAYNRYRIGNYKLNT

SEQ ID NO: 993 - protein, 15 aa, Artificial sequence
  Peptide
AYNRYRIGNYKLNTD

SEQ ID NO: 994 - protein, 15 aa, Artificial sequence
  Peptide
YNRYRIGNYKLNTDH

SEQ ID NO: 995 - protein, 15 aa, Artificial sequence
  Peptide
NRYRIGNYKLNTDHA

SEQ ID NO: 996 - protein, 15 aa, Artificial sequence
  Peptide
RYRIGNYKLNTDHAG

SEQ ID NO: 997 - protein, 15 aa, Artificial sequence
  Peptide
YRIGNYKLNTDHAGS

SEQ ID NO: 998 - protein, 15 aa, Artificial sequence
  Peptide
RIGNYKLNTDHAGSN

SEQ ID NO: 999 - protein, 15 aa, Artificial sequence
  Peptide
MFHLVDFQVTIAEIL

SEQ ID NO: 1000 - protein, 15 aa, Artificial sequence
  Peptide
FHLVDFQVTIAEILI

SEQ ID NO: 1001 - protein, 15 aa, Artificial sequence
  Peptide
HLVDFQVTIAEILII

SEQ ID NO: 1002 - protein, 15 aa, Artificial sequence
  Peptide
LVDFQVTIAEILIII

SEQ ID NO: 1003 - protein, 15 aa, Artificial sequence
  Peptide
VDFQVTIAEILIIIM

SEQ ID NO: 1004 - protein, 15 aa, Artificial sequence
  Peptide
DFQVTIAEILIIIMR

SEQ ID NO: 1005 - protein, 15 aa, Artificial sequence
  Peptide
FQVTIAEILIIIMRT

SEQ ID NO: 1006 - protein, 15 aa, Artificial sequence
  Peptide
QVTIAEILIIIMRTF

SEQ ID NO: 1007 - protein, 15 aa, Artificial sequence
  Peptide
VTIAEILIIIMRTFR

SEQ ID NO: 1008 - protein, 15 aa, Artificial sequence
  Peptide
TIAEILIIIMRTFRI

SEQ ID NO: 1009 - protein, 15 aa, Artificial sequence
  Peptide
IAEILIIIMRTFRIA

SEQ ID NO: 1010 - protein, 15 aa, Artificial sequence
  Peptide
TFRIAIWNLDVIISS

SEQ ID NO: 1011 - protein, 15 aa, Artificial sequence
  Peptide
FRIAIWNLDVIISSI

SEQ ID NO: 1012 - protein, 15 aa, Artificial sequence
  Peptide
RIAIWNLDVIISSIV

SEQ ID NO: 1013 - protein, 15 aa, Artificial sequence
  Peptide
IAIWNLDVIISSIVR

SEQ ID NO: 1014 - protein, 15 aa, Artificial sequence
  Peptide
AIWNLDVIISSIVRQ

SEQ ID NO: 1015 - protein, 15 aa, Artificial sequence
  Peptide
IWNLDVIISSIVRQL

SEQ ID NO: 1016 - protein, 15 aa, Artificial sequence
  Peptide
WNLDVIISSIVRQLF

SEQ ID NO: 1017 - protein, 15 aa, Artificial sequence
  Peptide
NLDVIISSIVRQLFK

SEQ ID NO: 1018 - protein, 15 aa, Artificial sequence
  Peptide
LDVIISSIVRQLFKP

SEQ ID NO: 1019 - protein, 15 aa, Artificial sequence
  Peptide
DVIISSIVRQLFKPL

SEQ ID NO: 1020 - protein, 15 aa, Artificial sequence
  Peptide
MKIILFLTLIVFTSC

SEQ ID NO: 1021 - protein, 15 aa, Artificial sequence
  Peptide
KIILFLTLIVFTSCE

SEQ ID NO: 1022 - protein, 15 aa, Artificial sequence
  Peptide
IILFLTLIVFTSCEL

SEQ ID NO: 1023 - protein, 15 aa, Artificial sequence
  Peptide
ILFLTLIVFTSCELY

SEQ ID NO: 1024 - protein, 15 aa, Artificial sequence
  Peptide
LFLTLIVFTSCELYH

SEQ ID NO: 1025 - protein, 15 aa, Artificial sequence
  Peptide
FLTLIVFTSCELYHY

SEQ ID NO: 1026 - protein, 15 aa, Artificial sequence
  Peptide
LTLIVFTSCELYHYQ

SEQ ID NO: 1027 - protein, 15 aa, Artificial sequence
  Peptide
TLIVFTSCELYHYQE

SEQ ID NO: 1028 - protein, 15 aa, Artificial sequence
  Peptide
LIVFTSCELYHYQEC

SEQ ID NO: 1029 - protein, 15 aa, Artificial sequence
  Peptide
IVFTSCELYHYQECV

SEQ ID NO: 1030 - protein, 15 aa, Artificial sequence
  Peptide
VFTSCELYHYQECVR

SEQ ID NO: 1031 - protein, 15 aa, Artificial sequence
  Peptide
FTSCELYHYQECVRG

SEQ ID NO: 1032 - protein, 15 aa, Artificial sequence
  Peptide
TSCELYHYQECVRGT

SEQ ID NO: 1033 - protein, 15 aa, Artificial sequence
  Peptide
SCELYHYQECVRGTT

SEQ ID NO: 1034 - protein, 15 aa, Artificial sequence
  Peptide
CELYHYQECVRGTTV

SEQ ID NO: 1035 - protein, 15 aa, Artificial sequence
  Peptide
VLLKEPCPSGTYEGN

SEQ ID NO: 1036 - protein, 15 aa, Artificial sequence
  Peptide
LLKEPCPSGTYEGNS

SEQ ID NO: 1037 - protein, 15 aa, Artificial sequence
  Peptide
LKEPCPSGTYEGNSP

SEQ ID NO: 1038 - protein, 15 aa, Artificial sequence
  Peptide
KEPCPSGTYEGNSPF

SEQ ID NO: 1039 - protein, 15 aa, Artificial sequence
  Peptide
EPCPSGTYEGNSPFH

SEQ ID NO: 1040 - protein, 15 aa, Artificial sequence
  Peptide
PCPSGTYEGNSPFHP

SEQ ID NO: 1041 - protein, 15 aa, Artificial sequence
  Peptide
NKFALTCTSTHFAFA

SEQ ID NO: 1042 - protein, 15 aa, Artificial sequence
  Peptide
KFALTCTSTHFAFAC

SEQ ID NO: 1043 - protein, 15 aa, Artificial sequence
  Peptide
FALTCTSTHFAFACA

SEQ ID NO: 1044 - protein, 15 aa, Artificial sequence
  Peptide
ALTCTSTHFAFACAD

SEQ ID NO: 1045 - protein, 15 aa, Artificial sequence
  Peptide
LTCTSTHFAFACADG

SEQ ID NO: 1046 - protein, 15 aa, Artificial sequence
  Peptide
TCTSTHFAFACADGT

SEQ ID NO: 1047 - protein, 15 aa, Artificial sequence
  Peptide
CTSTHFAFACADGTR

SEQ ID NO: 1048 - protein, 15 aa, Artificial sequence
  Peptide
TSTHFAFACADGTRH

SEQ ID NO: 1049 - protein, 15 aa, Artificial sequence
  Peptide
STHFAFACADGTRHT

SEQ ID NO: 1050 - protein, 15 aa, Artificial sequence
  Peptide
THFAFACADGTRHTY

SEQ ID NO: 1051 - protein, 15 aa, Artificial sequence
  Peptide
HFAFACADGTRHTYQ

SEQ ID NO: 1052 - protein, 15 aa, Artificial sequence
  Peptide
FAFACADGTRHTYQL

SEQ ID NO: 1053 - protein, 15 aa, Artificial sequence
  Peptide
AFACADGTRHTYQLR

SEQ ID NO: 1054 - protein, 15 aa, Artificial sequence
  Peptide
ARSVSPKLFIRQEEV

SEQ ID NO: 1055 - protein, 15 aa, Artificial sequence
  Peptide
RSVSPKLFIRQEEVQ

SEQ ID NO: 1056 - protein, 15 aa, Artificial sequence
  Peptide
SVSPKLFIRQEEVQQ

SEQ ID NO: 1057 - protein, 15 aa, Artificial sequence
  Peptide
VSPKLFIRQEEVQQE

SEQ ID NO: 1058 - protein, 15 aa, Artificial sequence
  Peptide
SPKLFIRQEEVQQEL

SEQ ID NO: 1059 - protein, 15 aa, Artificial sequence
  Peptide
PKLFIRQEEVQQELY

SEQ ID NO: 1060 - protein, 15 aa, Artificial sequence
  Peptide
KLFIRQEEVQQELYS

SEQ ID NO: 1061 - protein, 15 aa, Artificial sequence
  Peptide
LFIRQEEVQQELYSP

SEQ ID NO: 1062 - protein, 15 aa, Artificial sequence
  Peptide
QQELYSPLFLIVAAL

SEQ ID NO: 1063 - protein, 15 aa, Artificial sequence
  Peptide
QELYSPLFLIVAALV

SEQ ID NO: 1064 - protein, 15 aa, Artificial sequence
  Peptide
ELYSPLFLIVAALVF

SEQ ID NO: 1065 - protein, 15 aa, Artificial sequence
  Peptide
LYSPLFLIVAALVFL

SEQ ID NO: 1066 - protein, 15 aa, Artificial sequence
  Peptide
YSPLFLIVAALVFLI

SEQ ID NO: 1067 - protein, 15 aa, Artificial sequence
  Peptide
SPLFLIVAALVFLIL

SEQ ID NO: 1068 - protein, 15 aa, Artificial sequence
  Peptide
PLFLIVAALVFLILC

SEQ ID NO: 1069 - protein, 15 aa, Artificial sequence
  Peptide
LFLIVAALVFLILCF

SEQ ID NO: 1070 - protein, 15 aa, Artificial sequence
  Peptide
FLIVAALVFLILCFT

SEQ ID NO: 1071 - protein, 15 aa, Artificial sequence
  Peptide
LIVAALVFLILCFTI

SEQ ID NO: 1072 - protein, 15 aa, Artificial sequence
  Peptide
IVAALVFLILCFTIK

SEQ ID NO: 1073 - protein, 15 aa, Artificial sequence
  Peptide
VAALVFLILCFTIKR

SEQ ID NO: 1074 - protein, 15 aa, Artificial sequence
  Peptide
AALVFLILCFTIKRK

SEQ ID NO: 1075 - protein, 15 aa, Artificial sequence
  Peptide
ALVFLILCFTIKRKT

SEQ ID NO: 1076 - protein, 15 aa, Artificial sequence
  Peptide
LVFLILCFTIKRKTE

SEQ ID NO: 1077 - protein, 15 aa, Artificial sequence
  Peptide
MCLKILVRYNTRGNT

SEQ ID NO: 1078 - protein, 15 aa, Artificial sequence
  Peptide
CLKILVRYNTRGNTY

SEQ ID NO: 1079 - protein, 15 aa, Artificial sequence
  Peptide
LKILVRYNTRGNTYS

SEQ ID NO: 1080 - protein, 15 aa, Artificial sequence
  Peptide
KILVRYNTRGNTYST

SEQ ID NO: 1081 - protein, 15 aa, Artificial sequence
  Peptide
ILVRYNTRGNTYSTA

SEQ ID NO: 1082 - protein, 15 aa, Artificial sequence
  Peptide
LVRYNTRGNTYSTAW

SEQ ID NO: 1083 - protein, 15 aa, Artificial sequence
  Peptide
VRYNTRGNTYSTAWL

SEQ ID NO: 1084 - protein, 15 aa, Artificial sequence
  Peptide
RYNTRGNTYSTAWLC

SEQ ID NO: 1085 - protein, 15 aa, Artificial sequence
  Peptide
YNTRGNTYSTAWLCA

SEQ ID NO: 1086 - protein, 15 aa, Artificial sequence
  Peptide
NTRGNTYSTAWLCAL

SEQ ID NO: 1087 - protein, 15 aa, Artificial sequence
  Peptide
TRGNTYSTAWLCALG

SEQ ID NO: 1088 - protein, 15 aa, Artificial sequence
  Peptide
RGNTYSTAWLCALGK

SEQ ID NO: 1089 - protein, 15 aa, Artificial sequence
  Peptide
GNTYSTAWLCALGKV

SEQ ID NO: 1090 - protein, 15 aa, Artificial sequence
  Peptide
NTYSTAWLCALGKVL

SEQ ID NO: 1091 - protein, 15 aa, Artificial sequence
  Peptide
TYSTAWLCALGKVLP

SEQ ID NO: 1092 - protein, 15 aa, Artificial sequence
  Peptide
YSTAWLCALGKVLPF

SEQ ID NO: 1093 - protein, 15 aa, Artificial sequence
  Peptide
STAWLCALGKVLPFH

SEQ ID NO: 1094 - protein, 15 aa, Artificial sequence
  Peptide
TAWLCALGKVLPFHR

SEQ ID NO: 1095 - protein, 15 aa, Artificial sequence
  Peptide
AWLCALGKVLPFHRW

SEQ ID NO: 1096 - protein, 15 aa, Artificial sequence
  Peptide
WLCALGKVLPFHRWH

SEQ ID NO: 1097 - protein, 15 aa, Artificial sequence
  Peptide
LCALGKVLPFHRWHT

SEQ ID NO: 1098 - protein, 15 aa, Artificial sequence
  Peptide
CALGKVLPFHRWHTM

SEQ ID NO: 1099 - protein, 15 aa, Artificial sequence
  Peptide
ALGKVLPFHRWHTMV

SEQ ID NO: 1100 - protein, 15 aa, Artificial sequence
  Peptide
LGKVLPFHRWHTMVQ

SEQ ID NO: 1101 - protein, 15 aa, Artificial sequence
  Peptide
GKVLPFHRWHTMVQT

SEQ ID NO: 1102 - protein, 15 aa, Artificial sequence
  Peptide
KVLPFHRWHTMVQTC

SEQ ID NO: 1103 - protein, 15 aa, Artificial sequence
  Peptide
VLPFHRWHTMVQTCT

SEQ ID NO: 1104 - protein, 15 aa, Artificial sequence
  Peptide
LPFHRWHTMVQTCTP

SEQ ID NO: 1105 - protein, 15 aa, Artificial sequence
  Peptide
PFHRWHTMVQTCTPN

SEQ ID NO: 1106 - protein, 15 aa, Artificial sequence
  Peptide
FHRWHTMVQTCTPNV

SEQ ID NO: 1107 - protein, 15 aa, Artificial sequence
  Peptide
HRWHTMVQTCTPNVT

SEQ ID NO: 1108 - protein, 15 aa, Artificial sequence
  Peptide
VQTCTPNVTINCQDP

SEQ ID NO: 1109 - protein, 15 aa, Artificial sequence
  Peptide
QTCTPNVTINCQDPA

SEQ ID NO: 1110 - protein, 15 aa, Artificial sequence
  Peptide
TCTPNVTINCQDPAG

SEQ ID NO: 1111 - protein, 15 aa, Artificial sequence
  Peptide
CTPNVTINCQDPAGG

SEQ ID NO: 1112 - protein, 15 aa, Artificial sequence
  Peptide
TPNVTINCQDPAGGA

SEQ ID NO: 1113 - protein, 15 aa, Artificial sequence
  Peptide
DPAGGALIARCWYLH

SEQ ID NO: 1114 - protein, 15 aa, Artificial sequence
  Peptide
PAGGALIARCWYLHE

SEQ ID NO: 1115 - protein, 15 aa, Artificial sequence
  Peptide
AGGALIARCWYLHEG

SEQ ID NO: 1116 - protein, 15 aa, Artificial sequence
  Peptide
GGALIARCWYLHEGH

SEQ ID NO: 1117 - protein, 15 aa, Artificial sequence
  Peptide
GALIARCWYLHEGHQ

SEQ ID NO: 1118 - protein, 15 aa, Artificial sequence
  Peptide
ALIARCWYLHEGHQT

SEQ ID NO: 1119 - protein, 15 aa, Artificial sequence
  Peptide
LIARCWYLHEGHQTA

SEQ ID NO: 1120 - protein, 15 aa, Artificial sequence
  Peptide
QTAAFRDVLVVLNKR

SEQ ID NO: 1121 - protein, 15 aa, Artificial sequence
  Peptide
TAAFRDVLVVLNKRT

SEQ ID NO: 1122 - protein, 15 aa, Artificial sequence
  Peptide
AAFRDVLVVLNKRTN

SEQ ID NO: 1123 - protein, 15 aa, Artificial sequence
  Peptide
MSDNGPQSNQRSAPR

SEQ ID NO: 1124 - protein, 15 aa, Artificial sequence
  Peptide
SDNGPQSNQRSAPRI

SEQ ID NO: 1125 - protein, 15 aa, Artificial sequence
  Peptide
DNGPQSNQRSAPRIT

SEQ ID NO: 1126 - protein, 15 aa, Artificial sequence
  Peptide
TFGGPTDSTDNNQNG

SEQ ID NO: 1127 - protein, 15 aa, Artificial sequence
  Peptide
FGGPTDSTDNNQNGG

SEQ ID NO: 1128 - protein, 15 aa, Artificial sequence
  Peptide
GGPTDSTDNNQNGGR

SEQ ID NO: 1129 - protein, 15 aa, Artificial sequence
  Peptide
GPTDSTDNNQNGGRN

SEQ ID NO: 1130 - protein, 15 aa, Artificial sequence
  Peptide
PTDSTDNNQNGGRNG

SEQ ID NO: 1131 - protein, 15 aa, Artificial sequence
  Peptide
TDSTDNNQNGGRNGA

SEQ ID NO: 1132 - protein, 15 aa, Artificial sequence
  Peptide
DSTDNNQNGGRNGAR

SEQ ID NO: 1133 - protein, 15 aa, Artificial sequence
  Peptide
STDNNQNGGRNGARP

SEQ ID NO: 1134 - protein, 15 aa, Artificial sequence
  Peptide
TDNNQNGGRNGARPK

SEQ ID NO: 1135 - protein, 15 aa, Artificial sequence
  Peptide
DNNQNGGRNGARPKQ

SEQ ID NO: 1136 - protein, 15 aa, Artificial sequence
  Peptide
NNQNGGRNGARPKQR

SEQ ID NO: 1137 - protein, 15 aa, Artificial sequence
  Peptide
NQNGGRNGARPKQRR

SEQ ID NO: 1138 - protein, 15 aa, Artificial sequence
  Peptide
QNGGRNGARPKQRRP

SEQ ID NO: 1139 - protein, 15 aa, Artificial sequence
  Peptide
NGGRNGARPKQRRPQ

SEQ ID NO: 1140 - protein, 15 aa, Artificial sequence
  Peptide
GGRNGARPKQRRPQG

SEQ ID NO: 1141 - protein, 15 aa, Artificial sequence
  Peptide
GRNGARPKQRRPQGL

SEQ ID NO: 1142 - protein, 15 aa, Artificial sequence
  Peptide
RNGARPKQRRPQGLP

SEQ ID NO: 1143 - protein, 15 aa, Artificial sequence
  Peptide
NGARPKQRRPQGLPN

SEQ ID NO: 1144 - protein, 15 aa, Artificial sequence
  Peptide
GARPKQRRPQGLPNN

SEQ ID NO: 1145 - protein, 15 aa, Artificial sequence
  Peptide
ARPKQRRPQGLPNNT

SEQ ID NO: 1146 - protein, 15 aa, Artificial sequence
  Peptide
RPKQRRPQGLPNNTA

SEQ ID NO: 1147 - protein, 15 aa, Artificial sequence
  Peptide
PKQRRPQGLPNNTAS

SEQ ID NO: 1148 - protein, 15 aa, Artificial sequence
  Peptide
KQRRPQGLPNNTASW

SEQ ID NO: 1149 - protein, 15 aa, Artificial sequence
  Peptide
QRRPQGLPNNTASWF

SEQ ID NO: 1150 - protein, 15 aa, Artificial sequence
  Peptide
RRPQGLPNNTASWFT

SEQ ID NO: 1151 - protein, 15 aa, Artificial sequence
  Peptide
RPQGLPNNTASWFTA

SEQ ID NO: 1152 - protein, 15 aa, Artificial sequence
  Peptide
PQGLPNNTASWFTAL

SEQ ID NO: 1153 - protein, 15 aa, Artificial sequence
  Peptide
QGLPNNTASWFTALT

SEQ ID NO: 1154 - protein, 15 aa, Artificial sequence
  Peptide
GLPNNTASWFTALTQ

SEQ ID NO: 1155 - protein, 15 aa, Artificial sequence
  Peptide
LPNNTASWFTALTQH

SEQ ID NO: 1156 - protein, 15 aa, Artificial sequence
  Peptide
PNNTASWFTALTQHG

SEQ ID NO: 1157 - protein, 15 aa, Artificial sequence
  Peptide
NNTASWFTALTQHGK

SEQ ID NO: 1158 - protein, 15 aa, Artificial sequence
  Peptide
NTASWFTALTQHGKE

SEQ ID NO: 1159 - protein, 15 aa, Artificial sequence
  Peptide
TASWFTALTQHGKEE

SEQ ID NO: 1160 - protein, 15 aa, Artificial sequence
  Peptide
ASWFTALTQHGKEEL

SEQ ID NO: 1161 - protein, 15 aa, Artificial sequence
  Peptide
SWFTALTQHGKEELR

SEQ ID NO: 1162 - protein, 15 aa, Artificial sequence
  Peptide
WFTALTQHGKEELRF

SEQ ID NO: 1163 - protein, 15 aa, Artificial sequence
  Peptide
FTALTQHGKEELRFP

SEQ ID NO: 1164 - protein, 15 aa, Artificial sequence
  Peptide
TALTQHGKEELRFPR

SEQ ID NO: 1165 - protein, 15 aa, Artificial sequence
  Peptide
ALTQHGKEELRFPRG

SEQ ID NO: 1166 - protein, 15 aa, Artificial sequence
  Peptide
LTQHGKEELRFPRGQ

SEQ ID NO: 1167 - protein, 15 aa, Artificial sequence
  Peptide
TQHGKEELRFPRGQG

SEQ ID NO: 1168 - protein, 15 aa, Artificial sequence
  Peptide
QHGKEELRFPRGQGV

SEQ ID NO: 1169 - protein, 15 aa, Artificial sequence
  Peptide
HGKEELRFPRGQGVP

SEQ ID NO: 1170 - protein, 15 aa, Artificial sequence
  Peptide
GKEELRFPRGQGVPI

SEQ ID NO: 1171 - protein, 15 aa, Artificial sequence
  Peptide
KEELRFPRGQGVPIN

SEQ ID NO: 1172 - protein, 15 aa, Artificial sequence
  Peptide
EELRFPRGQGVPINT

SEQ ID NO: 1173 - protein, 15 aa, Artificial sequence
  Peptide
ELRFPRGQGVPINTN

SEQ ID NO: 1174 - protein, 15 aa, Artificial sequence
  Peptide
LRFPRGQGVPINTNS

SEQ ID NO: 1175 - protein, 15 aa, Artificial sequence
  Peptide
RFPRGQGVPINTNSG

SEQ ID NO: 1176 - protein, 15 aa, Artificial sequence
  Peptide
FPRGQGVPINTNSGP

SEQ ID NO: 1177 - protein, 15 aa, Artificial sequence
  Peptide
PRGQGVPINTNSGPD

SEQ ID NO: 1178 - protein, 15 aa, Artificial sequence
  Peptide
RGQGVPINTNSGPDD

SEQ ID NO: 1179 - protein, 15 aa, Artificial sequence
  Peptide
GQGVPINTNSGPDDQ

SEQ ID NO: 1180 - protein, 15 aa, Artificial sequence
  Peptide
QGVPINTNSGPDDQI

SEQ ID NO: 1181 - protein, 15 aa, Artificial sequence
  Peptide
GVPINTNSGPDDQIG

SEQ ID NO: 1182 - protein, 15 aa, Artificial sequence
  Peptide
VPINTNSGPDDQIGY

SEQ ID NO: 1183 - protein, 15 aa, Artificial sequence
  Peptide
PINTNSGPDDQIGYY

SEQ ID NO: 1184 - protein, 15 aa, Artificial sequence
  Peptide
INTNSGPDDQIGYYR

SEQ ID NO: 1185 - protein, 15 aa, Artificial sequence
  Peptide
NTNSGPDDQIGYYRR

SEQ ID NO: 1186 - protein, 15 aa, Artificial sequence
  Peptide
TNSGPDDQIGYYRRA

SEQ ID NO: 1187 - protein, 15 aa, Artificial sequence
  Peptide
NSGPDDQIGYYRRAT

SEQ ID NO: 1188 - protein, 15 aa, Artificial sequence
  Peptide
YYRRATRRVRGGDGK

SEQ ID NO: 1189 - protein, 15 aa, Artificial sequence
  Peptide
YRRATRRVRGGDGKM

SEQ ID NO: 1190 - protein, 15 aa, Artificial sequence
  Peptide
RRATRRVRGGDGKMK

SEQ ID NO: 1191 - protein, 15 aa, Artificial sequence
  Peptide
RATRRVRGGDGKMKE

SEQ ID NO: 1192 - protein, 15 aa, Artificial sequence
  Peptide
ATRRVRGGDGKMKEL

SEQ ID NO: 1193 - protein, 15 aa, Artificial sequence
  Peptide
TRRVRGGDGKMKELS

SEQ ID NO: 1194 - protein, 15 aa, Artificial sequence
  Peptide
RRVRGGDGKMKELSP

SEQ ID NO: 1195 - protein, 15 aa, Artificial sequence
  Peptide
RVRGGDGKMKELSPR

SEQ ID NO: 1196 - protein, 15 aa, Artificial sequence
  Peptide
VRGGDGKMKELSPRW

SEQ ID NO: 1197 - protein, 15 aa, Artificial sequence
  Peptide
RGGDGKMKELSPRWY

SEQ ID NO: 1198 - protein, 15 aa, Artificial sequence
  Peptide
GGDGKMKELSPRWYF

SEQ ID NO: 1199 - protein, 15 aa, Artificial sequence
  Peptide
GDGKMKELSPRWYFY

SEQ ID NO: 1200 - protein, 15 aa, Artificial sequence
  Peptide
DGKMKELSPRWYFYY

SEQ ID NO: 1201 - protein, 15 aa, Artificial sequence
  Peptide
GKMKELSPRWYFYYL

SEQ ID NO: 1202 - protein, 15 aa, Artificial sequence
  Peptide
KMKELSPRWYFYYLG

SEQ ID NO: 1203 - protein, 15 aa, Artificial sequence
  Peptide
MKELSPRWYFYYLGT

SEQ ID NO: 1204 - protein, 15 aa, Artificial sequence
  Peptide
KELSPRWYFYYLGTG

SEQ ID NO: 1205 - protein, 15 aa, Artificial sequence
  Peptide
ELSPRWYFYYLGTGP

SEQ ID NO: 1206 - protein, 15 aa, Artificial sequence
  Peptide
LSPRWYFYYLGTGPE

SEQ ID NO: 1207 - protein, 15 aa, Artificial sequence
  Peptide
SPRWYFYYLGTGPEA

SEQ ID NO: 1208 - protein, 15 aa, Artificial sequence
  Peptide
PRWYFYYLGTGPEAS

SEQ ID NO: 1209 - protein, 15 aa, Artificial sequence
  Peptide
RWYFYYLGTGPEASL

SEQ ID NO: 1210 - protein, 15 aa, Artificial sequence
  Peptide
WYFYYLGTGPEASLP

SEQ ID NO: 1211 - protein, 15 aa, Artificial sequence
  Peptide
YFYYLGTGPEASLPY

SEQ ID NO: 1212 - protein, 15 aa, Artificial sequence
  Peptide
FYYLGTGPEASLPYG

SEQ ID NO: 1213 - protein, 15 aa, Artificial sequence
  Peptide
YYLGTGPEASLPYGA

SEQ ID NO: 1214 - protein, 15 aa, Artificial sequence
  Peptide
YLGTGPEASLPYGAN

SEQ ID NO: 1215 - protein, 15 aa, Artificial sequence
  Peptide
LGTGPEASLPYGANK

SEQ ID NO: 1216 - protein, 15 aa, Artificial sequence
  Peptide
GTGPEASLPYGANKE

SEQ ID NO: 1217 - protein, 15 aa, Artificial sequence
  Peptide
TGPEASLPYGANKEG

SEQ ID NO: 1218 - protein, 15 aa, Artificial sequence
  Peptide
GPEASLPYGANKEGI

SEQ ID NO: 1219 - protein, 15 aa, Artificial sequence
  Peptide
PEASLPYGANKEGIV

SEQ ID NO: 1220 - protein, 15 aa, Artificial sequence
  Peptide
EASLPYGANKEGIVW

SEQ ID NO: 1221 - protein, 15 aa, Artificial sequence
  Peptide
ASLPYGANKEGIVWV

SEQ ID NO: 1222 - protein, 15 aa, Artificial sequence
  Peptide
SLPYGANKEGIVWVA

SEQ ID NO: 1223 - protein, 15 aa, Artificial sequence
  Peptide
LPYGANKEGIVWVAT

SEQ ID NO: 1224 - protein, 15 aa, Artificial sequence
  Peptide
PYGANKEGIVWVATE

SEQ ID NO: 1225 - protein, 15 aa, Artificial sequence
  Peptide
YGANKEGIVWVATEG

SEQ ID NO: 1226 - protein, 15 aa, Artificial sequence
  Peptide
GANKEGIVWVATEGA

SEQ ID NO: 1227 - protein, 15 aa, Artificial sequence
  Peptide
ANKEGIVWVATEGAL

SEQ ID NO: 1228 - protein, 15 aa, Artificial sequence
  Peptide
NKEGIVWVATEGALN

SEQ ID NO: 1229 - protein, 15 aa, Artificial sequence
  Peptide
KEGIVWVATEGALNT

SEQ ID NO: 1230 - protein, 15 aa, Artificial sequence
  Peptide
EGIVWVATEGALNTP

SEQ ID NO: 1231 - protein, 15 aa, Artificial sequence
  Peptide
GIVWVATEGALNTPK

SEQ ID NO: 1232 - protein, 15 aa, Artificial sequence
  Peptide
IVWVATEGALNTPKD

SEQ ID NO: 1233 - protein, 15 aa, Artificial sequence
  Peptide
VWVATEGALNTPKDH

SEQ ID NO: 1234 - protein, 15 aa, Artificial sequence
  Peptide
WVATEGALNTPKDHI

SEQ ID NO: 1235 - protein, 15 aa, Artificial sequence
  Peptide
VATEGALNTPKDHIG

SEQ ID NO: 1236 - protein, 15 aa, Artificial sequence
  Peptide
ATEGALNTPKDHIGT

SEQ ID NO: 1237 - protein, 15 aa, Artificial sequence
  Peptide
TEGALNTPKDHIGTR

SEQ ID NO: 1238 - protein, 15 aa, Artificial sequence
  Peptide
EGALNTPKDHIGTRN

SEQ ID NO: 1239 - protein, 15 aa, Artificial sequence
  Peptide
GALNTPKDHIGTRNP

SEQ ID NO: 1240 - protein, 15 aa, Artificial sequence
  Peptide
ALNTPKDHIGTRNPN

SEQ ID NO: 1241 - protein, 15 aa, Artificial sequence
  Peptide
LNTPKDHIGTRNPNN

SEQ ID NO: 1242 - protein, 15 aa, Artificial sequence
  Peptide
NTPKDHIGTRNPNNN

SEQ ID NO: 1243 - protein, 15 aa, Artificial sequence
  Peptide
TPKDHIGTRNPNNNA

SEQ ID NO: 1244 - protein, 15 aa, Artificial sequence
  Peptide
PKDHIGTRNPNNNAA

SEQ ID NO: 1245 - protein, 15 aa, Artificial sequence
  Peptide
KDHIGTRNPNNNAAT

SEQ ID NO: 1246 - protein, 15 aa, Artificial sequence
  Peptide
DHIGTRNPNNNAATV

SEQ ID NO: 1247 - protein, 15 aa, Artificial sequence
  Peptide
HIGTRNPNNNAATVL

SEQ ID NO: 1248 - protein, 15 aa, Artificial sequence
  Peptide
IGTRNPNNNAATVLQ

SEQ ID NO: 1249 - protein, 15 aa, Artificial sequence
  Peptide
GTRNPNNNAATVLQL

SEQ ID NO: 1250 - protein, 15 aa, Artificial sequence
  Peptide
TRNPNNNAATVLQLP

SEQ ID NO: 1251 - protein, 15 aa, Artificial sequence
  Peptide
RNPNNNAATVLQLPQ

SEQ ID NO: 1252 - protein, 15 aa, Artificial sequence
  Peptide
NPNNNAATVLQLPQG

SEQ ID NO: 1253 - protein, 15 aa, Artificial sequence
  Peptide
PNNNAATVLQLPQGT

SEQ ID NO: 1254 - protein, 15 aa, Artificial sequence
  Peptide
NNNAATVLQLPQGTT

SEQ ID NO: 1255 - protein, 15 aa, Artificial sequence
  Peptide
LPKGFYAEGSRGGSQ

SEQ ID NO: 1256 - protein, 15 aa, Artificial sequence
  Peptide
PKGFYAEGSRGGSQA

SEQ ID NO: 1257 - protein, 15 aa, Artificial sequence
  Peptide
KGFYAEGSRGGSQAS

SEQ ID NO: 1258 - protein, 15 aa, Artificial sequence
  Peptide
GFYAEGSRGGSQASS

SEQ ID NO: 1259 - protein, 15 aa, Artificial sequence
  Peptide
FYAEGSRGGSQASSR

SEQ ID NO: 1260 - protein, 15 aa, Artificial sequence
  Peptide
YAEGSRGGSQASSRS

SEQ ID NO: 1261 - protein, 15 aa, Artificial sequence
  Peptide
AEGSRGGSQASSRSS

SEQ ID NO: 1262 - protein, 15 aa, Artificial sequence
  Peptide
EGSRGGSQASSRSSS

SEQ ID NO: 1263 - protein, 15 aa, Artificial sequence
  Peptide
GSRGGSQASSRSSSR

SEQ ID NO: 1264 - protein, 15 aa, Artificial sequence
  Peptide
SRGGSQASSRSSSRS

SEQ ID NO: 1265 - protein, 15 aa, Artificial sequence
  Peptide
RGGSQASSRSSSRSR

SEQ ID NO: 1266 - protein, 15 aa, Artificial sequence
  Peptide
GGSQASSRSSSRSRG

SEQ ID NO: 1267 - protein, 15 aa, Artificial sequence
  Peptide
GSQASSRSSSRSRGN

SEQ ID NO: 1268 - protein, 15 aa, Artificial sequence
  Peptide
SQASSRSSSRSRGNS

SEQ ID NO: 1269 - protein, 15 aa, Artificial sequence
  Peptide
QASSRSSSRSRGNSR

SEQ ID NO: 1270 - protein, 15 aa, Artificial sequence
  Peptide
ASSRSSSRSRGNSRN

SEQ ID NO: 1271 - protein, 15 aa, Artificial sequence
  Peptide
SSRSSSRSRGNSRNS

SEQ ID NO: 1272 - protein, 15 aa, Artificial sequence
  Peptide
SRSSSRSRGNSRNST

SEQ ID NO: 1273 - protein, 15 aa, Artificial sequence
  Peptide
RSSSRSRGNSRNSTP

SEQ ID NO: 1274 - protein, 15 aa, Artificial sequence
  Peptide
SSSRSRGNSRNSTPG

SEQ ID NO: 1275 - protein, 15 aa, Artificial sequence
  Peptide
SSRSRGNSRNSTPGS

SEQ ID NO: 1276 - protein, 15 aa, Artificial sequence
  Peptide
SRSRGNSRNSTPGSS

SEQ ID NO: 1277 - protein, 15 aa, Artificial sequence
  Peptide
RSRGNSRNSTPGSSR

SEQ ID NO: 1278 - protein, 15 aa, Artificial sequence
  Peptide
SRGNSRNSTPGSSRG

SEQ ID NO: 1279 - protein, 15 aa, Artificial sequence
  Peptide
RGNSRNSTPGSSRGN

SEQ ID NO: 1280 - protein, 15 aa, Artificial sequence
  Peptide
GNSRNSTPGSSRGNS

SEQ ID NO: 1281 - protein, 15 aa, Artificial sequence
  Peptide
NSRNSTPGSSRGNSP

SEQ ID NO: 1282 - protein, 15 aa, Artificial sequence
  Peptide
SRNSTPGSSRGNSPA

SEQ ID NO: 1283 - protein, 15 aa, Artificial sequence
  Peptide
SSRGNSPARMASGGG

SEQ ID NO: 1284 - protein, 15 aa, Artificial sequence
  Peptide
SRGNSPARMASGGGE

SEQ ID NO: 1285 - protein, 15 aa, Artificial sequence
  Peptide
RGNSPARMASGGGET

SEQ ID NO: 1286 - protein, 15 aa, Artificial sequence
  Peptide
GNSPARMASGGGETA

SEQ ID NO: 1287 - protein, 15 aa, Artificial sequence
  Peptide
SGGGETALALLLLDR

SEQ ID NO: 1288 - protein, 15 aa, Artificial sequence
  Peptide
GGGETALALLLLDRL

SEQ ID NO: 1289 - protein, 15 aa, Artificial sequence
  Peptide
GGETALALLLLDRLN

SEQ ID NO: 1290 - protein, 15 aa, Artificial sequence
  Peptide
GETALALLLLDRLNQ

SEQ ID NO: 1291 - protein, 15 aa, Artificial sequence
  Peptide
ETALALLLLDRLNQL

SEQ ID NO: 1292 - protein, 15 aa, Artificial sequence
  Peptide
TALALLLLDRLNQLE

SEQ ID NO: 1293 - protein, 15 aa, Artificial sequence
  Peptide
ALALLLLDRLNQLES

SEQ ID NO: 1294 - protein, 15 aa, Artificial sequence
  Peptide
LALLLLDRLNQLESK

SEQ ID NO: 1295 - protein, 15 aa, Artificial sequence
  Peptide
ALLLLDRLNQLESKV

SEQ ID NO: 1296 - protein, 15 aa, Artificial sequence
  Peptide
LLLLDRLNQLESKVS

SEQ ID NO: 1297 - protein, 15 aa, Artificial sequence
  Peptide
LLLDRLNQLESKVSG

SEQ ID NO: 1298 - protein, 15 aa, Artificial sequence
  Peptide
LLDRLNQLESKVSGK

SEQ ID NO: 1299 - protein, 15 aa, Artificial sequence
  Peptide
LDRLNQLESKVSGKG

SEQ ID NO: 1300 - protein, 15 aa, Artificial sequence
  Peptide
DRLNQLESKVSGKGQ

SEQ ID NO: 1301 - protein, 15 aa, Artificial sequence
  Peptide
RLNQLESKVSGKGQQ

SEQ ID NO: 1302 - protein, 15 aa, Artificial sequence
  Peptide
LNQLESKVSGKGQQQ

SEQ ID NO: 1303 - protein, 15 aa, Artificial sequence
  Peptide
NQLESKVSGKGQQQQ

SEQ ID NO: 1304 - protein, 15 aa, Artificial sequence
  Peptide
QLESKVSGKGQQQQG

SEQ ID NO: 1305 - protein, 15 aa, Artificial sequence
  Peptide
LESKVSGKGQQQQGQ

SEQ ID NO: 1306 - protein, 15 aa, Artificial sequence
  Peptide
ESKVSGKGQQQQGQT

SEQ ID NO: 1307 - protein, 15 aa, Artificial sequence
  Peptide
SKVSGKGQQQQGQTV

SEQ ID NO: 1308 - protein, 15 aa, Artificial sequence
  Peptide
KVSGKGQQQQGQTVT

SEQ ID NO: 1309 - protein, 15 aa, Artificial sequence
  Peptide
VSGKGQQQQGQTVTK

SEQ ID NO: 1310 - protein, 15 aa, Artificial sequence
  Peptide
SGKGQQQQGQTVTKK

SEQ ID NO: 1311 - protein, 15 aa, Artificial sequence
  Peptide
GKGQQQQGQTVTKKS

SEQ ID NO: 1312 - protein, 15 aa, Artificial sequence
  Peptide
KGQQQQGQTVTKKSA

SEQ ID NO: 1313 - protein, 15 aa, Artificial sequence
  Peptide
GQQQQGQTVTKKSAA

SEQ ID NO: 1314 - protein, 15 aa, Artificial sequence
  Peptide
QQQQGQTVTKKSAAE

SEQ ID NO: 1315 - protein, 15 aa, Artificial sequence
  Peptide
QQQGQTVTKKSAAEA

SEQ ID NO: 1316 - protein, 15 aa, Artificial sequence
  Peptide
AEASKKPRQKRTATK

SEQ ID NO: 1317 - protein, 15 aa, Artificial sequence
  Peptide
EASKKPRQKRTATKQ

SEQ ID NO: 1318 - protein, 15 aa, Artificial sequence
  Peptide
ASKKPRQKRTATKQY

SEQ ID NO: 1319 - protein, 15 aa, Artificial sequence
  Peptide
SKKPRQKRTATKQYN

SEQ ID NO: 1320 - protein, 15 aa, Artificial sequence
  Peptide
KKPRQKRTATKQYNV

SEQ ID NO: 1321 - protein, 15 aa, Artificial sequence
  Peptide
QAFGRRGPEQTQGNF

SEQ ID NO: 1322 - protein, 15 aa, Artificial sequence
  Peptide
AFGRRGPEQTQGNFG

SEQ ID NO: 1323 - protein, 15 aa, Artificial sequence
  Peptide
TQGNFGDQDLIRQGT

SEQ ID NO: 1324 - protein, 15 aa, Artificial sequence
  Peptide
QGNFGDQDLIRQGTD

SEQ ID NO: 1325 - protein, 15 aa, Artificial sequence
  Peptide
GNFGDQDLIRQGTDY

SEQ ID NO: 1326 - protein, 15 aa, Artificial sequence
  Peptide
NFGDQDLIRQGTDYK

SEQ ID NO: 1327 - protein, 15 aa, Artificial sequence
  Peptide
FGDQDLIRQGTDYKH

SEQ ID NO: 1328 - protein, 15 aa, Artificial sequence
  Peptide
GDQDLIRQGTDYKHW

SEQ ID NO: 1329 - protein, 15 aa, Artificial sequence
  Peptide
DQDLIRQGTDYKHWP

SEQ ID NO: 1330 - protein, 15 aa, Artificial sequence
  Peptide
QDLIRQGTDYKHWPQ

SEQ ID NO: 1331 - protein, 15 aa, Artificial sequence
  Peptide
DLIRQGTDYKHWPQI

SEQ ID NO: 1332 - protein, 15 aa, Artificial sequence
  Peptide
LIRQGTDYKHWPQIA

SEQ ID NO: 1333 - protein, 15 aa, Artificial sequence
  Peptide
IRQGTDYKHWPQIAQ

SEQ ID NO: 1334 - protein, 15 aa, Artificial sequence
  Peptide
RQGTDYKHWPQIAQF

SEQ ID NO: 1335 - protein, 15 aa, Artificial sequence
  Peptide
QGTDYKHWPQIAQFA

SEQ ID NO: 1336 - protein, 15 aa, Artificial sequence
  Peptide
GTDYKHWPQIAQFAP

SEQ ID NO: 1337 - protein, 15 aa, Artificial sequence
  Peptide
TDYKHWPQIAQFAPS

SEQ ID NO: 1338 - protein, 15 aa, Artificial sequence
  Peptide
DYKHWPQIAQFAPSA

SEQ ID NO: 1339 - protein, 15 aa, Artificial sequence
  Peptide
YKHWPQIAQFAPSAS

SEQ ID NO: 1340 - protein, 15 aa, Artificial sequence
  Peptide
KHWPQIAQFAPSASA

SEQ ID NO: 1341 - protein, 15 aa, Artificial sequence
  Peptide
HWPQIAQFAPSASAF

SEQ ID NO: 1342 - protein, 15 aa, Artificial sequence
  Peptide
WPQIAQFAPSASAFF

SEQ ID NO: 1343 - protein, 15 aa, Artificial sequence
  Peptide
PQIAQFAPSASAFFG

SEQ ID NO: 1344 - protein, 15 aa, Artificial sequence
  Peptide
QIAQFAPSASAFFGM

SEQ ID NO: 1345 - protein, 15 aa, Artificial sequence
  Peptide
IAQFAPSASAFFGMS

SEQ ID NO: 1346 - protein, 15 aa, Artificial sequence
  Peptide
AQFAPSASAFFGMSR

SEQ ID NO: 1347 - protein, 15 aa, Artificial sequence
  Peptide
QFAPSASAFFGMSRI

SEQ ID NO: 1348 - protein, 15 aa, Artificial sequence
  Peptide
FAPSASAFFGMSRIG

SEQ ID NO: 1349 - protein, 15 aa, Artificial sequence
  Peptide
APSASAFFGMSRIGM

SEQ ID NO: 1350 - protein, 15 aa, Artificial sequence
  Peptide
PSASAFFGMSRIGME

SEQ ID NO: 1351 - protein, 15 aa, Artificial sequence
  Peptide
SASAFFGMSRIGMEV

SEQ ID NO: 1352 - protein, 15 aa, Artificial sequence
  Peptide
ASAFFGMSRIGMEVT

SEQ ID NO: 1353 - protein, 15 aa, Artificial sequence
  Peptide
SAFFGMSRIGMEVTP

SEQ ID NO: 1354 - protein, 15 aa, Artificial sequence
  Peptide
AFFGMSRIGMEVTPS

SEQ ID NO: 1355 - protein, 15 aa, Artificial sequence
  Peptide
FFGMSRIGMEVTPSG

SEQ ID NO: 1356 - protein, 15 aa, Artificial sequence
  Peptide
FGMSRIGMEVTPSGT

SEQ ID NO: 1357 - protein, 15 aa, Artificial sequence
  Peptide
GMSRIGMEVTPSGTW

SEQ ID NO: 1358 - protein, 15 aa, Artificial sequence
  Peptide
MSRIGMEVTPSGTWL

SEQ ID NO: 1359 - protein, 15 aa, Artificial sequence
  Peptide
SRIGMEVTPSGTWLT

SEQ ID NO: 1360 - protein, 15 aa, Artificial sequence
  Peptide
RIGMEVTPSGTWLTY

SEQ ID NO: 1361 - protein, 15 aa, Artificial sequence
  Peptide
IGMEVTPSGTWLTYH

SEQ ID NO: 1362 - protein, 15 aa, Artificial sequence
  Peptide
GMEVTPSGTWLTYHG

SEQ ID NO: 1363 - protein, 15 aa, Artificial sequence
  Peptide
MEVTPSGTWLTYHGA

SEQ ID NO: 1364 - protein, 15 aa, Artificial sequence
  Peptide
EVTPSGTWLTYHGAI

SEQ ID NO: 1365 - protein, 15 aa, Artificial sequence
  Peptide
VTPSGTWLTYHGAIK

SEQ ID NO: 1366 - protein, 15 aa, Artificial sequence
  Peptide
TPSGTWLTYHGAIKL

SEQ ID NO: 1367 - protein, 15 aa, Artificial sequence
  Peptide
PSGTWLTYHGAIKLD

SEQ ID NO: 1368 - protein, 15 aa, Artificial sequence
  Peptide
SGTWLTYHGAIKLDD

SEQ ID NO: 1369 - protein, 15 aa, Artificial sequence
  Peptide
GTWLTYHGAIKLDDK

SEQ ID NO: 1370 - protein, 15 aa, Artificial sequence
  Peptide
TWLTYHGAIKLDDKD

SEQ ID NO: 1371 - protein, 15 aa, Artificial sequence
  Peptide
WLTYHGAIKLDDKDP

SEQ ID NO: 1372 - protein, 15 aa, Artificial sequence
  Peptide
LTYHGAIKLDDKDPQ

SEQ ID NO: 1373 - protein, 15 aa, Artificial sequence
  Peptide
TYHGAIKLDDKDPQF

SEQ ID NO: 1374 - protein, 15 aa, Artificial sequence
  Peptide
YHGAIKLDDKDPQFK

SEQ ID NO: 1375 - protein, 15 aa, Artificial sequence
  Peptide
HGAIKLDDKDPQFKD

SEQ ID NO: 1376 - protein, 15 aa, Artificial sequence
  Peptide
GAIKLDDKDPQFKDN

SEQ ID NO: 1377 - protein, 15 aa, Artificial sequence
  Peptide
AIKLDDKDPQFKDNV

SEQ ID NO: 1378 - protein, 15 aa, Artificial sequence
  Peptide
FKDNVILLNKHIDAY

SEQ ID NO: 1379 - protein, 15 aa, Artificial sequence
  Peptide
KDNVILLNKHIDAYK

SEQ ID NO: 1380 - protein, 15 aa, Artificial sequence
  Peptide
DNVILLNKHIDAYKT

SEQ ID NO: 1381 - protein, 15 aa, Artificial sequence
  Peptide
NVILLNKHIDAYKTF

SEQ ID NO: 1382 - protein, 15 aa, Artificial sequence
  Peptide
VILLNKHIDAYKTFP

SEQ ID NO: 1383 - protein, 15 aa, Artificial sequence
  Peptide
ILLNKHIDAYKTFPP

SEQ ID NO: 1384 - protein, 15 aa, Artificial sequence
  Peptide
LLNKHIDAYKTFPPT

SEQ ID NO: 1385 - protein, 15 aa, Artificial sequence
  Peptide
LNKHIDAYKTFPPTE

SEQ ID NO: 1386 - protein, 15 aa, Artificial sequence
  Peptide
NKHIDAYKTFPPTEP

SEQ ID NO: 1387 - protein, 15 aa, Artificial sequence
  Peptide
KHIDAYKTFPPTEPK

SEQ ID NO: 1388 - protein, 15 aa, Artificial sequence
  Peptide
HIDAYKTFPPTEPKK

SEQ ID NO: 1389 - protein, 15 aa, Artificial sequence
  Peptide
IDAYKTFPPTEPKKD

SEQ ID NO: 1390 - protein, 15 aa, Artificial sequence
  Peptide
DAYKTFPPTEPKKDK

SEQ ID NO: 1391 - protein, 15 aa, Artificial sequence
  Peptide
AYKTFPPTEPKKDKK

SEQ ID NO: 1392 - protein, 15 aa, Artificial sequence
  Peptide
YKTFPPTEPKKDKKK

SEQ ID NO: 1393 - protein, 15 aa, Artificial sequence
  Peptide
KTFPPTEPKKDKKKK

SEQ ID NO: 1394 - protein, 15 aa, Artificial sequence
  Peptide
TFPPTEPKKDKKKKT

SEQ ID NO: 1395 - protein, 15 aa, Artificial sequence
  Peptide
FPPTEPKKDKKKKTD

SEQ ID NO: 1396 - protein, 15 aa, Artificial sequence
  Peptide
PPTEPKKDKKKKTDE

SEQ ID NO: 1397 - protein, 15 aa, Artificial sequence
  Peptide
PTEPKKDKKKKTDEA

SEQ ID NO: 1398 - protein, 15 aa, Artificial sequence
  Peptide
TEPKKDKKKKTDEAQ

SEQ ID NO: 1399 - protein, 15 aa, Artificial sequence
  Peptide
EPKKDKKKKTDEAQP

SEQ ID NO: 1400 - protein, 15 aa, Artificial sequence
  Peptide
PKKDKKKKTDEAQPL

SEQ ID NO: 1401 - protein, 15 aa, Artificial sequence
  Peptide
KKDKKKKTDEAQPLP

SEQ ID NO: 1402 - protein, 15 aa, Artificial sequence
  Peptide
KDKKKKTDEAQPLPQ

SEQ ID NO: 1403 - protein, 15 aa, Artificial sequence
  Peptide
DKKKKTDEAQPLPQR

SEQ ID NO: 1404 - protein, 15 aa, Artificial sequence
  Peptide
KKKKTDEAQPLPQRQ

SEQ ID NO: 1405 - protein, 15 aa, Artificial sequence
  Peptide
KKKTDEAQPLPQRQK

SEQ ID NO: 1406 - protein, 15 aa, Artificial sequence
  Peptide
KKTDEAQPLPQRQKK

SEQ ID NO: 1407 - protein, 15 aa, Artificial sequence
  Peptide
KTDEAQPLPQRQKKQ

SEQ ID NO: 1408 - protein, 15 aa, Artificial sequence
  Peptide
TDEAQPLPQRQKKQP

SEQ ID NO: 1409 - protein, 15 aa, Artificial sequence
  Peptide
DEAQPLPQRQKKQPT

SEQ ID NO: 1410 - protein, 15 aa, Artificial sequence
  Peptide
EAQPLPQRQKKQPTV

SEQ ID NO: 1411 - protein, 15 aa, Artificial sequence
  Peptide
AQPLPQRQKKQPTVT

SEQ ID NO: 1412 - protein, 15 aa, Artificial sequence
  Peptide
KKQPTVTLLPAADMD

SEQ ID NO: 1413 - protein, 15 aa, Artificial sequence
  Peptide
KQPTVTLLPAADMDD

SEQ ID NO: 1414 - protein, 15 aa, Artificial sequence
  Peptide
QPTVTLLPAADMDDF

SEQ ID NO: 1415 - protein, 15 aa, Artificial sequence
  Peptide
PTVTLLPAADMDDFS

SEQ ID NO: 1416 - protein, 15 aa, Artificial sequence
  Peptide
TVTLLPAADMDDFSR

SEQ ID NO: 1417 - protein, 15 aa, Artificial sequence
  Peptide
VTLLPAADMDDFSRQ

SEQ ID NO: 1418 - protein, 15 aa, Artificial sequence
  Peptide
TLLPAADMDDFSRQL

SEQ ID NO: 1419 - protein, 15 aa, Artificial sequence
  Peptide
LLPAADMDDFSRQLQ

SEQ ID NO: 1420 - protein, 15 aa, Artificial sequence
  Peptide
LPAADMDDFSRQLQN

SEQ ID NO: 1421 - protein, 15 aa, Artificial sequence
  Peptide
PAADMDDFSRQLQNS

SEQ ID NO: 1422 - protein, 15 aa, Artificial sequence
  Peptide
AADMDDFSRQLQNSM

SEQ ID NO: 1423 - protein, 15 aa, Artificial sequence
  Peptide
ADMDDFSRQLQNSMS

SEQ ID NO: 1424 - protein, 15 aa, Artificial sequence
  Peptide
DMDDFSRQLQNSMSG

SEQ ID NO: 1425 - protein, 15 aa, Artificial sequence
  Peptide
MDDFSRQLQNSMSGA

SEQ ID NO: 1426 - protein, 15 aa, Artificial sequence
  Peptide
DDFSRQLQNSMSGAS

SEQ ID NO: 1427 - protein, 15 aa, Artificial sequence
  Peptide
DFSRQLQNSMSGASA

SEQ ID NO: 1428 - protein, 15 aa, Artificial sequence
  Peptide
FSRQLQNSMSGASAD

SEQ ID NO: 1429 - protein, 15 aa, Artificial sequence
  Peptide
SRQLQNSMSGASADS

SEQ ID NO: 1430 - protein, 15 aa, Artificial sequence
  Peptide
RQLQNSMSGASADST

SEQ ID NO: 1431 - protein, 15 aa, Artificial sequence
  Peptide
QLQNSMSGASADSTQ

SEQ ID NO: 1432 - protein, 15 aa, Artificial sequence
  Peptide
LQNSMSGASADSTQA

(19) United States
(12) Patent Application Publication
(10) Pub. No.: US 2007/0128217 A1
(43) Pub. Date: Jun. 7, 2007

(54) Antigenic peptides of SARS coronavirus and uses thereof

(75) Inventors: Jan H. ter Meulen, Amsterdam (NL); Jaap Goudsmit, Amsterdam (NL); Jelle Wouter Slootstra, Lelystad (NL); Peter Timmerman, Lelystad (NL); Cornelis Adriaan de Kruif, De Bilt (NL); Edward N. van den Brink, Halfweg (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/332,820

(22) Filed: Jan. 13, 2006

Related U.S. Application Data
(63) Continuation of application No. PCT/EP04/51498, filed on Jul. 15, 2004.

(30) Foreign Application Priority Data
Jul. 15, 2003 (WO) PCT/EP03/50308
Jul. 24, 2003 (WO) PCT/EP03/50333
Sep. 2, 2003 (WO) PCT/EP03/50392
Oct. 27, 2003 (WO) PCT/EP03/50761
Nov. 24, 2003 (WO) PCT/EP03/50883

(51) Int. Cl. C12Q 1/68; A61K 39/12

(52) U.S. Cl. 424/204.1; 435/6

(57) ABSTRACT

The present invention pertains to antigenic peptides of SARS-CoV and their use in diagnostic test methods and in the treatment of condition resulting from SARS-CoV. Furthermore, this invention provides antibodies capable of specifically recognizing the peptides of the invention. The antibodies can also advantageously be used in diagnostic test methods and in the treatment of condition resulting from SARS-CoV.

### CROSS-REFERENCE TO RELATED APPLICATIONS

This application is a continuation of PCT International Patent Application No. PCT/EP2004/051498, filed on Jul. 15, 2004, designating the United States of America, and published, in English, as PCT International Publication No. WO 2005/012337 A2 on Feb. 10, 2005, which application claims priority to International Patent Application No. PCT/EP03/50883 filed Nov. 24, 2003, which claims priority to International Patent Application No. PCT/EP03/50761 filed Oct. 27, 2003, which claims priority to International Patent Application No. PCT/EP03/50392 filed Sep. 2, 2003, which claims priority to International Patent Application No. PCT/EP03/50333 filed Jul. 24, 2003, which in turn claims priority to International Patent Application No. PCT/EP03/50308 filed Jul. 15, 2003, the contents of the entirety of each of which are incorporated by this reference.

### STATEMENT ACCORDING TO 37 C.F.R. § 1.52(e)(5) SEQUENCE LISTING SUBMITTED ON COMPACT DISC

Pursuant to 37 C.F.R. § 1.52(e)(1)(ii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “2578-7587US seq list” which is 395 KB and created on Mar. 13, 2006.

### FIELD OF THE INVENTION

Various embodiments generally relate to biotechnology. More specifically, various embodiments relate to medicine. Even more specifically, various embodiments relate to antigenic peptides of SARS coronavirus and uses thereof.

### BACKGROUND OF THE INVENTION

Recently, a new and in several cases deadly clinical syndrome was observed in the human population, now called severe acute respiratory syndrome (SARS) (Holmes, 2003). The syndrome is caused by a novel coronavirus (Ksiazek et al., 2003), referred to as the SARS-CoV. The genome sequence of SARS-CoV has been determined (Rota et al., 2003; Marra et al., 2003). However, much remains to be learnt about this virus, and means and methods for diagnostics and treatment of the virus and the syndrome are needed. The present invention provides means and methods for use in diagnostics, treatment and prevention of SARS-CoV.

### SUMMARY OF THE INVENTION

The present invention pertains to antigenic peptides of SARS-CoV. Furthermore, the invention provides fusion proteins comprising these peptides and antibodies against these peptides. The use of the peptides, fusion proteins and antibodies in the treatment of a condition resulting from SARS-CoV and a diagnostic test method for determining the presence of antibodies recognizing SARS-CoV in a sample or for determining the presence of SARS-CoV in a sample are also contemplated in the present invention.

### DETAILED DESCRIPTION OF THE INVENTION

In a first aspect, the invention provides antigenic peptides of SARS-CoV, particularly the SARS-CoV strain called Urbani. In the present invention, binding of sera from SARS patients to a series of overlapping 15-mer peptides, which were either in linear form or in looped/cyclic form, of the proteins from SARS-CoV Urbani was analyzed by means of PEPSCAN analysis (see inter alia WO 84/03564, WO 93/09872, Slootstra et al. 1996). The complete genome of SARS-CoV Urbani can be found under EMBL-database accession number AY278741, “SARS coronavirus Urbani, complete genome.” The coding sequence (CDS) of the proteins of SARS-CoV Urbani is also shown under EMBL-database accession number AY278741. In the present invention is disclosed that several of the SARS-CoV Urbani proteins (or potential proteins) called protein X1 (the protein-id of protein X1 is AAP13446, see also SEQ ID NO:1), protein X2 (the protein-id of protein X2 is AAP13447, see also SEQ ID NO:2), E protein (the protein-id of the envelope protein, E protein, is AAP13443, see also SEQ ID NO:3), M protein (the protein-id of the small membrane protein, M protein, is AAP13444, see also SEQ ID NO:4), protein X3 (the protein-id of protein X3 is AAP13448, see also SEQ ID NO:5), protein X4 (the protein-id of protein X4 is AAP13449, see also SEQ ID NO:6), protein X5 (the protein-id of protein X5 is AAP13450, see also SEQ ID NO:7), and N protein (the protein-id of the nucleocapsid protein, N protein, is AAP13445, see also SEQ ID NO:8) comprise antigenic peptides.

The complete genome and the amino acid sequence of (potential) proteins of other SARS-CoV strains including, but not limited to, TOR2, Frankfurt 1 and HSR 1 can also be found in the EMBL-database. The accession number in the EMBL-database of the complete genome of the strains TOR2, Frankfurt 1 and HSR 1 is AY274119, AY291315 and AY323977, respectively. Under these accession numbers the amino acid sequence of (potential) proteins of these strains can also be found. Because the Urbani proteins indicated above are also found in identical or highly homologous form in other SARS-CoV strains, the antigenic peptides found in the present invention may not only be used for detection of the SARS-CoV strain Urbani and the prevention and/or treatment of a condition resulting from the SARS-CoV strain Urbani, but may also be useful in detecting SARS-CoV in general and preventing and/or treating a condition resulting from SARS-CoV in general.

In one embodiment, the invention provides a peptide having an amino acid sequence selected from the group consisting of RFFTLGSITAQPVKI (SEQ ID NO:9), FFTLGSITAQPVKID (SEQ ID NO:10), FTLGSITAQPVKIDN (SEQ ID NO:11), TLGSITAQPVKIDNA (SEQ ID NO:12), LGSITAQPVKIDNAS (SEQ ID NO:13), GSITAQPVKIDNASP (SEQ ID NO:14), SITAQPVKIDNASPA (SEQ ID NO:15), ITAQPVKIDNASPAS (SEQ ID NO:16), TAQPVKIDNASPAST (SEQ ID NO:17), AQPVKIDNASPASTV (SEQ ID NO:18), QPVKIDNASPASTVH (SEQ ID NO:19), PVKIDNASPASTVHA (SEQ ID NO:20), VKIDNASPASTVHAT (SEQ ID NO:21), KIDNASPASTVHATA (SEQ ID NO:22), IDNASPASTVHATAT (SEQ ID NO:23), DNASPASTVHATATI (SEQ ID NO:24), NASPASTVHATATIP (SEQ ID NO:25), ASPASTVHATATIPL (SEQ ID NO:26), SPASTVHATATIPLQ (SEQ ID NO:27), PASTVHATATIPLQA (SEQ ID NO:28), ASTVHATATIPLQAS (SEQ ID NO:29), STVHATATIPLQASL (SEQ ID NO:30), TVHATATIPLQASLP (SEQ ID NO:31), VHATATIPLQASLPF (SEQ ID NO:32), INACRIIMRCWLCWK (SEQ ID NO:33), NACRIIMRCWLCWKC (SEQ ID NO:34), ACRIIMRCWLCWKCK (SEQ ID NO:35), CRIIMRCWLCWKCKS (SEQ ID NO:36), RIIMRCWLCWKCKSK (SEQ ID NO:37), IIMRCWLCWKCKSKN (SEQ ID NO:38), IMRCWLCWKCKSKNP (SEQ ID NO:39), MRCWLCWKCKSKNPL (SEQ ID NO:40), RCWLCWKCKSKNPLL (SEQ ID NO:41), CWLCWKCKSKNPLLY (SEQ ID NO:42), WLCWKCKSKNPLLYD (SEQ ID NO:43), LCWKCKSKNPLLYDA (SEQ ID NO:44), CWKCKSKNPLLYDAN (SEQ ID NO:45), YDANYFVCWHTHNYD (SEQ ID NO:46), DANYFVCWHTHNYDY (SEQ ID NO:47), ANYFVCWHTHNYDYC (SEQ ID NO:48), NYFVCWHTHNYDYCI (SEQ ID NO:49), YFVCWHTHNYDYCIP (SEQ ID NO:50), FVCWHTHNYDYCIPY (SEQ ID NO:51), VCWHTHNYDYCIPYN (SEQ ID NO:52), CWHTHNYDYCIPYNS (SEQ ID NO:53), WHTHNYDYCIPYNSV (SEQ ID NO:54), HTHNYDYCIPYNSVT (SEQ ID NO:55), THNYDYCIPYNSVTD (SEQ ID NO:56), HNYDYCIPYNSVTDT (SEQ ID NO:57), NYDYCIPYNSVTDTI (SEQ ID NO:58), YDYCIPYNSVTDTIV (SEQ ID NO:59), DYCIPYNSVTDTIVV (SEQ ID NO:60), YCIPYNSVTDTIVVT (SEQ ID NO:61), GDGISTPKLKEDYQI (SEQ ID NO:62), DGISTPKLKEDYQIG (SEQ ID NO:63), GISTPKLKEDYQIGG (SEQ ID NO:64), ISTPKLKEDYQIGGY (SEQ ID NO:65), STPKLKEDYQIGGYS (SEQ ID NO:66), TPKLKEDYQIGGYSE (SEQ ID NO:67), PKLKEDYQIGGYSED (SEQ ID NO:68), KLKEDYQIGGYSEDR (SEQ ID NO:69), LKEDYQIGGYSEDRH (SEQ ID NO:70), KEDYQIGGYSEDRHS (SEQ ID NO:71), EDYQIGGYSEDRHSG (SEQ ID NO:72), DYQIGGYSEDRHSGV (SEQ ID NO:73), YQIGGYSEDRHSGVK (SEQ ID NO:74), QIGGYSEDRHSGVKD (SEQ ID NO:75), IGGYSEDRHSGVKDY (SEQ ID NO:76), GGYSEDRHSGVKDYV (SEQ ID NO:77), GYSEDRHSGVKDYVV (SEQ ID NO:78), YSEDRHSGVKDYVVV (SEQ ID NO:79), SEDRHSGVKDYVVVH (SEQ ID NO:80), EDRHSGVKDYVVVHG (SEQ ID NO:81), DRHSGVKDYVVVHGY (SEQ ID NO:82), RHSGVKDYVVVHGYF (SEQ ID NO:83), HSGVKDYVVVHGYFT (SEQ ID NO:84), SGVKDYVVVHGYFTE (SEQ ID NO:85), GVKDYVVVHGYFTEV (SEQ ID NO:86), ATFFIFNKLVKDPPN (SEQ ID NO:87), TFFIFNKLVKDPPNV (SEQ ID NO:88), FFIFNKLVKDPPNVQ (SEQ ID NO:89), FIFNKLVKDPPNVQI (SEQ ID NO:90), IFNKLVKDPPNVQIH (SEQ ID NO:91), FNKLVKDPPNVQIHT (SEQ ID NO:92), NKLVKDPPNVQIHTI (SEQ ID NO:93), KLVKDPPNVQIHTID (SEQ ID NO:94), LVKDPPNVQIHTIDG (SEQ ID NO:95), VKDPPNVQIHTIDGS (SEQ ID NO:96), KDPPNVQIHTIDGSS (SEQ ID NO:97), DGSSGVANPAMDPIY (SEQ ID NO:98), GSSGVANPAMDPIYD (SEQ ID NO:99), SSGVANPAMDPIYDE (SEQ ID NO:100), SGVANPAMDPIYDEP (SEQ ID NO:101), GVANPAMDPIYDEPT (SEQ ID NO:102), VANPAMDPIYDEPTT (SEQ ID NO:103), ANPAMDPIYDEPTTT (SEQ ID NO:104), NPAMDPIYDEPTTTT (SEQ ID NO:105), PAMDPIYDEPTTTTS (SEQ ID NO:106), AMDPIYDEPTTTTSV (SEQ ID NO:107), MDPIYDEPTTTTSVP (SEQ ID NO:108), DPIYDEPTTTTSVPL (SEQ ID NO:109), MMPTTLFAGTHITMT (SEQ ID NO:110), MPTTLFAGTHITMTT (SEQ ID NO:111), PTTLFAGTHITMTTV (SEQ ID NO:112), TTLFAGTHITMTTVY (SEQ ID NO:113), TLFAGTHITMTTVYH (SEQ ID NO:114), LFAGTHITMTTVYHI (SEQ ID NO:115), FAGTHITMTTVYHIT (SEQ ID NO:116), AGTHITMTTVYHITV (SEQ ID NO:117), GTHITMTTVYHITVS (SEQ ID NO:118), FQHQNSKKTTKLVVI (SEQ ID NO:119), QHQNSKKTTKLVVIL (SEQ ID NO:120), HQNSKKTTKLVVILR (SEQ ID NO:121), QNSKKTTKLVVILRI (SEQ ID NO:122), NSKKTTKLVVILRIG (SEQ ID NO:123), SKKTTKLVVILRIGT (SEQ ID NO:124), KKTTKLVVILRIGTQ (SEQ ID NO:125), KTTKLVVILRIGTQV (SEQ ID NO:126), TTKLVVILRIGTQVL (SEQ ID NO:127), TKLVVILRIGTQVLK (SEQ ID NO:128), KLVVILRIGTQVLKT (SEQ ID NO:129), LRIGTQVLKTMSLYM (SEQ ID NO:130), RIGTQVLKTMSLYMA (SEQ ID NO:131), IGTQVLKTMSLYMAI (SEQ ID NO:132), GTQVLKTMSLYMAIS (SEQ ID NO:133), TQVLKTMSLYMAISP (SEQ ID NO:134), QVLKTMSLYMAISPK (SEQ ID NO:135), VLKTMSLYMAISPKF (SEQ ID NO:136), LKTMSLYMAISPKFT (SEQ ID NO:137), KTMSLYMAISPKFTT (SEQ ID NO:138), MMSRRRLLACLCKHK (SEQ ID NO:139), MSRRRLLACLCKHKK (SEQ ID NO:140), SRRRLLACLCKHKKV (SEQ ID NO:141), RRRLLACLCKHKKVS (SEQ ID NO:142), RRLLACLCKHKKVST (SEQ ID NO:143), RLLACLCKHKKVSTN (SEQ ID NO:144), LLACLCKHKKVSTNL (SEQ ID NO:145), LACLCKHKKVSTNLC (SEQ ID NO:146), ACLCKHKKVSTNLCT (SEQ ID NO:147), CLCKHKKVSTNLCTH (SEQ ID NO:148), LCKHKKVSTNLCTHS (SEQ ID NO:149), CKHKKVSTNLCTHSF (SEQ ID NO:150), KHKKVSTNLCTHSFR (SEQ ID NO:151), HKKVSTNLCTHSFRK (SEQ ID NO:152), KKVSTNLCTHSFRKK (SEQ ID NO:153), KVSTNLCTHSFRKKQ (SEQ ID NO:154), VSTNLCTHSFRKKQV (SEQ ID NO:155), STNLCTHSFRKKQVR (SEQ ID NO:156), LCAYCCNIVNVSLVK (SEQ ID NO:157), CAYCCNIVNVSLVKP (SEQ ID NO:158), AYCCNIVNVSLVKPT (SEQ ID NO:159), YCCNIVNVSLVKPTV (SEQ ID NO:160), CCNIVNVSLVKPTVY (SEQ ID NO:161), CNIVNVSLVKPTVYV (SEQ ID NO:162), NIVNVSLVKPTVYVY (SEQ ID NO:163), IVNVSLVKPTVYVYS (SEQ ID NO:164), VNVSLVKPTVYVYSR (SEQ ID NO:165), NVSLVKPTVYVYSRV (SEQ ID NO:166), VSLVKPTVYVYSRVK (SEQ ID NO:167), SLVKPTVYVYSRVKN (SEQ ID NO:168), LVKPTVYVYSRVKNL (SEQ ID NO:169), VKPTVYVYSRVKNLN (SEQ ID NO:170), KPTVYVYSRVKNLNS (SEQ ID NO:171), PTVYVYSRVKNLNSS (SEQ ID NO:172), TVYVYSRVKNLNSSE (SEQ ID NO:173), VYVYSRVKNLNSSEG (SEQ ID NO:174), YVYSRVKNLNSSEGV (SEQ ID NO:175), VYSRVKNLNSSEGVP (SEQ ID NO:176), YSRVKNLNSSEGVPD (SEQ ID NO:177), SRVKNLNSSEGVPDL (SEQ ID NO:178), RVKNLNSSEGVPDLL (SEQ ID NO:179), VKNLNSSEGVPDLLV (SEQ ID NO:180), MADNGTITVEELKQL (SEQ ID NO:181), ADNGTITVEELKQLL (SEQ ID NO:182), DNGTITVEELKQLLE (SEQ ID NO:183), NGTITVEELKQLLEQ (SEQ ID NO:184), GTITVEELKQLLEQW (SEQ ID NO:185), TITVEELKQLLEQWN (SEQ ID NO:186), ITVEELKQLLEQWNL (SEQ ID NO:187), TVEELKQLLEQWNLV (SEQ ID NO:188), VEELKQLLEQWNLVI (SEQ ID NO:189), EELKQLLEQWNLVIG (SEQ ID NO:190), QFAYSNRNRFLYIIK (SEQ ID NO:191), FAYSNRNRFLYIIKL (SEQ ID NO:192), AYSNRNRFLYIIKLV (SEQ ID NO:193), YSNRNRFLYIIKLVF (SEQ ID NO:194), SNRNRFLYIIKLVFL (SEQ ID NO:195), NRNRFLYIIKLVFLW (SEQ ID NO:196), RNRFLYIIKLVFLWL (SEQ ID NO:197), NRFLYIIKLVFLWLL (SEQ ID NO:198), RFLYIIKLVFLWLLW (SEQ ID NO:199), FLYIIKLVFLWLLWP (SEQ ID NO:200), INWVTGGIAIAMACI (SEQ ID NO:201), NWVTGGIAIAMACIV (SEQ ID NO:202), WVTGGIAIAMACIVG (SEQ ID NO:203), VTGGIAIAMACIVGL (SEQ ID NO:204), TGGIAIAMACIVGLM (SEQ ID NO:205), GGIAIAMACIVGLMW (SEQ ID NO:206), GIAIAMACIVGLMWL (SEQ ID NO:207), IAIAMACIVGLMWLS (SEQ ID NO:208), LMWLSYFVASFRLFA (SEQ ID NO:209), MWLSYFVASFRLFAR (SEQ ID NO:210), WLSYFVASFRLFART (SEQ ID NO:211), LSYFVASFRLFARTR (SEQ ID NO:212), SYFVASFRLFARTRS (SEQ ID NO:213), YFVASFRLFARTRSM (SEQ ID NO:214), FVASFRLFARTRSMW (SEQ ID NO:215), VASFRLFARTRSMWS (SEQ ID NO:216), NILLNVPLRGTIVTR (SEQ ID NO:217), ILLNVPLRGTIVTRP (SEQ ID NO:218), LLNVPLRGTIVTRPL (SEQ ID NO:219), LNVPLRGTIVTRPLM (SEQ ID NO:220), NVPLRGTIVTRPLME (SEQ ID NO:221), VPLRGTIVTRPLMES (SEQ ID NO:222), PLRGTIVTRPLMESE (SEQ ID NO:223), LRGTIVTRPLMESEL (SEQ ID NO:224), RGTIVTRPLMESELV (SEQ ID NO:225), GTIVTRPLMESELVI (SEQ ID NO:226), TIVTRPLMESELVIG (SEQ ID NO:227), IVTRPLMESELVIGA (SEQ ID NO:229), VTRPLMESELVIGAV (SEQ ID NO:230), TRPLMESELVIGAVI (SEQ ID NO:231), RPLMESELVIGAVII (SEQ ID NO:232), VIGAVIIRGHLRMAG (SEQ ID NO:233), IGAVIIRGHLRMAGH (SEQ ID NO:234), GAVIIRGHLRMAGHP (SEQ ID NO:235), AVIIRGHLRMAGHPL (SEQ ID NO:236), VIIRGHLRMAGHPLG (SEQ ID NO:237), IIRGHLRMAGHPLGR (SEQ ID NO:238), IRGHLRMAGHPLGRC (SEQ ID NO:239), RGHLRMAGHPLGRCD (SEQ ID NO:240), GHLRMAGHPLGRCDI (SEQ ID NO:241), HLRMAGHPLGRCDIK (SEQ ID NO:242), LRMAGHPLGRCDIKD (SEQ ID NO:243), RMAGHPLGRCDIKDL (SEQ ID NO:244), MAGHPLGRCDIKDLP (SEQ ID NO:245), AGHPLGRCDIKDLPK (SEQ ID NO:246), GHPLGRCDIKDLPKE (SEQ ID NO:247), HPLGRCDIKDLPKEI (SEQ ID NO:248), PLGRCDIKDLPKEIT (SEQ ID NO:249), LGRCDIKDLPKEITV (SEQ ID NO:250), GRCDIKDLPKEITVA (SEQ ID NO:251), TLSYYKLGASQRVGT (SEQ ID NO:252), LSYYKLGASQRVGTD (SEQ ID NO:253), SYYKLGASQRVGTDS (SEQ ID NO:254), YYKLGASQRVGTDSG (SEQ ID NO:255), YKLGASQRVGTDSGF (SEQ ID NO:256), KLGASQRVGTDSGFA (SEQ ID NO:257), LGASQRVGTDSGFAA (SEQ ID NO:258), GASQRVGTDSGFAAY (SEQ ID NO:259), ASQRVGTDSGFAAYN (SEQ ID NO:260), IGNYKLNTDHAGSND (SEQ ID NO:261), GNYKLNTDHAGSNDN (SEQ ID NO:262), NYKLNTDHAGSNDNI (SEQ ID NO:263), YKLNTDHAGSNDNIA (SEQ ID NO:264), KLNTDHAGSNDNIAL (SEQ ID NO:265), LNTDHAGSNDNIALL (SEQ ID NO:266), NTDHAGSNDNIALLV (SEQ ID NO:267), TDHAGSNDNIALLVQ (SEQ ID NO:268), AEILIIIMRTFRIAI (SEQ ID NO:269), EILIIIMRTFRIAIW (SEQ ID NO:270), ILIIIMRTFRIAIWN (SEQ ID NO:271), LIIIMRTFRIAIWNL (SEQ ID NO:272), IIIMRTFRIAIWNLD (SEQ ID NO:273), IIMRTFRIAIWNLDV (SEQ ID NO:274), IMRTFRIAIWNLDVI (SEQ ID NO:275), MRTFRIAIWNLDVII (SEQ ID NO:276), RTFRIAIWNLDVIIS (SEQ ID NO:277), VIISSIVRQLFKPLT (SEQ ID NO:278), IISSIVRQLFKPLTK (SEQ ID NO:279), ISSIVRQLFKPLTKK (SEQ ID NO:280), SSIVRQLFKPLTKKN (SEQ ID NO:281), SIVRQLFKPLTKKNY (SEQ ID NO:282), IVRQLFKPLTKKNYS (SEQ ID NO:283), VRQLFKPLTKKNYSE (SEQ ID NO:284), RQLFKPLTKKNYSEL (SEQ ID NO:285), QLFKPLTKKNYSELD (SEQ ID NO:286), LFKPLTKKNYSELDD (SEQ ID NO:287), FKPLTKKNYSELDDE (SEQ ID NO:288), KPLTKKNYSELDDEE (SEQ ID NO:289), PLTKKNYSELDDEEP (SEQ ID NO:290), LTKKNYSELDDEEPM (SEQ ID NO:291), TKKNYSELDDEEPME (SEQ ID NO:292), KKNYSELDDEEPMEL (SEQ ID NO:293), KNYSELDDEEPMELD (SEQ ID NO:294), NYSELDDEEPMELDY (SEQ ID NO:295), YSELDDEEPMELDYP (SEQ ID NO:296), ELYHYQECVRGTTVL (SEQ ID NO:297), LYHYQECVRGTTVLL (SEQ ID NO:298), YHYQECVRGTTVLLK (SEQ ID NO:299), HYQECVRGTTVLLKE (SEQ ID NO:300), YQECVRGTTVLLKEP (SEQ ID NO:301), QECVRGTTVLLKEPC (SEQ ID NO:302), ECVRGTTVLLKEPCP (SEQ ID NO:303), CVRGTTVLLKEPCPS (SEQ ID NO:304), VRGTTVLLKEPCPSG (SEQ ID NO:305), RGTTVLLKEPCPSGT (SEQ ID NO:306), GTTVLLKEPCPSGTY (SEQ ID NO:307), TTVLLKEPCPSGTYE (SEQ ID NO:308), TVLLKEPCPSGTYEG (SEQ ID NO:309), CPSGTYEGNSPFHPL (SEQ ID NO:310), PSGTYEGNSPFHPLA (SEQ ID NO:311), SGTYEGNSPFHPLAD (SEQ ID NO:312), GTYEGNSPFHPLADN (SEQ ID NO:313), TYEGNSPFHPLADNK (SEQ ID NO:314), YEGNSPFHPLADNKF (SEQ ID NO:315), EGNSPFHPLADNKFA (SEQ ID NO:316), GNSPFHPLADNKFAL (SEQ ID NO:317), NSPFHPLADNKFALT (SEQ ID NO:318), SPFHPLADNKFALTC (SEQ ID NO:319), PFHPLADNKFALTCT (SEQ ID NO:320), FHPLADNKFALTCTS (SEQ ID NO:321), HPLADNKFALTCTST (SEQ ID NO:322), PLADNKFALTCTSTH (SEQ ID NO:323), LADNKFALTCTSTHF (SEQ ID NO:324), ADNKFALTCTSTHFA (SEQ ID NO:325), DNKFALTCTSTHFAF (SEQ ID NO:326), FIRQEEVQQELYSPL (SEQ ID NO:327), IRQEEVQQELYSPLF (SEQ ID NO:328), RQEEVQQELYSPLFL (SEQ ID NO:329), QEEVQQELYSPLFLI (SEQ ID NO:330), EEVQQELYSPLFLIV (SEQ ID NO:331), EVQQELYSPLFLIVA (SEQ ID NO:332), VQQELYSPLFLIVAA (SEQ ID NO:333), RWHTMVQTCTPNVTI (SEQ ID NO:334), WHTMVQTCTPNVTIN (SEQ ID NO:335), HTMVQTCTPNVTINC (SEQ ID NO:336), TMVQTCTPNVTINCQ (SEQ ID NO:337), MVQTCTPNVTINCQD (SEQ ID NO:338), PNVTINCQDPAGGAL (SEQ ID NO:339), NVTINCQDPAGGALI (SEQ ID NO:340), VTINCQDPAGGALIA (SEQ ID NO:341), TINCQDPAGGALIAR (SEQ ID NO:342), INCQDPAGGALIARC (SEQ ID NO:343), NCQDPAGGALIARCW (SEQ ID NO:344), CQDPAGGALIARCWY (SEQ ID NO:345), QDPAGGALIARCWYL (SEQ ID NO:346), IARCWYLHEGHQTAA (SEQ ID NO:347), ARCWYLHEGHQTAAF (SEQ ID NO:348), RCWYLHEGHQTAAFR (SEQ ID NO:349), CWYLHEGHQTAAFRD (SEQ ID NO:350), WYLHEGHQTAAFRDV (SEQ ID NO:351), YLHEGHQTAAFRDVL (SEQ ID NO:352), LHEGHQTAAFRDVLV (SEQ ID NO:353), HEGHQTAAFRDVLVV (SEQ ID NO:354), EGHQTAAFRDVLVVL (SEQ ID NO:355), GHQTAAFRDVLVVLN (SEQ ID NO:356), HQTAAFRDVLVVLNK (SEQ ID NO:357), NNAATVLQLPQGTTL (SEQ ID NO:358), NAATVLQLPQGTTLP (SEQ ID NO:359), AATVLQLPQGTTLPK (SEQ ID NO:360), ATVLQLPQGTTLPKG (SEQ ID NO:361), TVLQLPQGTTLPKGF (SEQ ID NO:362), VLQLPQGTTLPKGFY (SEQ ID NO:363), LQLPQGTTLPKGFYA (SEQ ID NO:364), QLPQGTTLPKGFYAE (SEQ ID NO:365), LPQGTTLPKGFYAEG (SEQ ID NO:366), PQGTTLPKGFYAEGS (SEQ ID NO:367), QGTTLPKGFYAEGSR (SEQ ID NO:368), GTTLPKGFYAEGSRG (SEQ ID NO:369), TTLPKGFYAEGSRGG (SEQ ID NO:370), TLPKGFYAEGSRGGS (SEQ ID NO:371), NSPARMASGGGETAL (SEQ ID NO:372), SPARMASGGGETALA (SEQ ID NO:373), PARMASGGGETALAL (SEQ ID NO:374), ARMASGGGETALALL (SEQ ID NO:375), RMASGGGETALALLL (SEQ ID NO:376), MASGGGETALALLLL (SEQ ID NO:377), ASGGGETALALLLLD (SEQ ID NO:378), QQGQTVTKKSAAEAS (SEQ ID NO:379), QGQTVTKKSAAEASK (SEQ ID NO:380), GQTVTKKSAAEASKK (SEQ ID NO:381), QTVTKKSAAEASKKP (SEQ ID NO:382), TVTKKSAAEASKKPR (SEQ ID NO:383), VTKKSAAEASKKPRQ (SEQ ID NO:384), TKKSAAEASKKPRQK (SEQ ID NO:385), KKSAAEASKKPRQKR (SEQ ID NO:386), KSAAEASKKPRQKRT (SEQ ID NO:387), SAAEASKKPRQKRTA (SEQ ID NO:388), AAEASKKPRQKRTAT (SEQ ID NO:389), KPRQKRTATKQYNVT (SEQ ID NO:390), PRQKRTATKQYNVTQ (SEQ ID NO:391), RQKRTATKQYNVTQA (SEQ ID NO:392), QKRTATKQYNVTQAF (SEQ ID NO:393), KRTATKQYNVTQAFG (SEQ ID NO:394), RTATKQYNVTQAFGR (SEQ ID NO:395), TATKQYNVTQAFGRR (SEQ ID NO:396), FGRRGPEQTQGNFGD (SEQ ID NO:397), GRRGPEQTQGNFGDQ (SEQ ID NO:398), RRGPEQTQGNFGDQD (SEQ ID NO:399), RGPEQTQGNFGDQDL (SEQ ID NO:400), GPEQTQGNFGDQDLI (SEQ ID NO:401), PEQTQGNFGDQDLIR (SEQ ID NO:402), EQTQGNFGDQDLIRQ (SEQ ID NO:403), QTQGNFGDQDLIRQG (SEQ ID NO:404), IKLDDKDPQFKDNVI (SEQ ID NO:405), KLDDKDPQFKDNVIL (SEQ ID NO:406), LDDKDPQFKDNVILL (SEQ ID NO:407), DDKDPQFKDNVILLN (SEQ ID NO:408), DKDPQFKDNVILLNK (SEQ ID NO:409), KDPQFKDNVILLNKH (SEQ ID NO:410), DPQFKDNVILLNKHI (SEQ ID NO:411), PQFKDNVILLNKHID (SEQ ID NO:412), QFKDNVILLNKHIDA (SEQ ID NO:413), QPLPQRQKKQPTVTL (SEQ ID NO:414), PLPQRQKKQPTVTLL (SEQ ID NO:415), LPQRQKKQPTVTLLP (SEQ ID NO:416), PQRQKKQPTVTLLPA (SEQ ID NO:417), QRQKKQPTVTLLPAA (SEQ ID NO:418), RQKKQPTVTLLPAAD (SEQ ID NO:419) and QKKQPTVTLLPAADM (SEQ ID NO:420).

The peptides above are recognized in linear and/or looped/cyclic form by at least one of the following sera: serum derived from an individual that has been infected by SARS-CoV and has recovered from SARS (serum called SARS-green); serum derived from an individual in which the virus was still detectable by PCR and who suffered a prolonged and severe form of the illness (serum called SARS-yellow); sera derived from individuals which have been and/or are infected by SARS-CoV (sera called 1a (individual 1, early serum), 1b (individual 1, late serum) and 2 (individual 2), 6 (individual 6), 37 (individual 37), 62 (individual 62) and London. It is clear for a person skilled in the art that the term “individuals that have been infected by SARS-CoV” as used herein also encompasses individuals that have been infected by SARS-CoV and are recovered from SARS.

In an embodiment of the invention, the invention encompasses a peptide having an amino acid sequence selected from the group consisting of RFFTLGSITAQPVKI (SEQ ID NO:9), FFTLGSITAQPVKID (SEQ ID NO:10), FTLGSITAQPVKIDN (SEQ ID NO:11), TLGSITAQPVKIDNA (SEQ ID NO:12), LGSITAQPVKIDNAS (SEQ ID NO:13), GSITAQPVKIDNASP (SEQ ID NO:14), SITAQPVKIDNASPA (SEQ ID NO:15), ITAQPVKIDNASPAS (SEQ ID NO:16), TAQPVKIDNASPAST (SEQ ID NO:17), AQPVKIDNASPASTV (SEQ ID NO:18), QPVKIDNASPASTVH (SEQ ID NO:19), PVKIDNASPASTVHA (SEQ ID NO:20), VKIDNASPASTVHAT (SEQ ID NO:21), KIDNASPASTVHATA (SEQ ID NO:22), IDNASPASTVHATAT (SEQ ID NO:23), DNASPASTVHATATI (SEQ ID NO:24), NASPASTVHATATIP (SEQ ID NO:25), ASPASTVHATATIPL (SEQ ID NO:26), SPASTVHATATIPLQ (SEQ ID NO:27), PASTVHATATIPLQA (SEQ ID NO:28), ASTVHATATIPLQAS (SEQ ID NO:29), STVHATATIPLQASL (SEQ ID NO:30), TVHATATIPLQASLP (SEQ ID NO:31), VHATATIPLQASLPF (SEQ ID NO:32), INACRIIMRCWLCWK (SEQ ID NO:33), NACRIIMRCWLCWKC (SEQ ID NO:34), ACRIIMRCWLCWKCK (SEQ ID NO:35), CRIIMRCWLCWKCKS (SEQ ID NO:36), RIIMRCWLCWKCKSK (SEQ ID NO:37), IIMRCWLCWKCKSKN (SEQ ID NO:38), IMRCWLCWKCKSKNP (SEQ ID NO:39), MRCWLCWKCKSKNPL (SEQ ID NO:40), RCWLCWKCKSKNPLL (SEQ ID NO:41), CWLCWKCKSKNPLLY (SEQ ID NO:42), WLCWKCKSKNPLLYD (SEQ ID NO:43), LCWKCKSKNPLLYDA (SEQ ID NO:44), CWKCKSKNPLLYDAN (SEQ ID NO:45), YDANYFVCWHTHNYD (SEQ ID NO:46), DANYFVCWHTHNYDY (SEQ ID NO:47), ANYFVCWHTHNYDYC (SEQ ID NO:48), NYFVCWHTHNYDYCI (SEQ ID NO:49), YFVCWHTHNYDYCIP (SEQ ID NO:50), FVCWHTHNYDYCIPY (SEQ ID NO:51), VCWHTHNYDYCIPYN (SEQ ID NO:52), CWHTHNYDYCIPYNS (SEQ ID NO:53), WHTHNYDYCIPYNSV (SEQ ID NO:54), HTHNYDYCIPYNSVT (SEQ ID NO:55), THNYDYCIPYNSVTD (SEQ ID NO:56), HNYDYCIPYNSVTDT (SEQ ID NO:57), NYDYCIPYNSVTDTI (SEQ ID NO:58), YDYCIPYNSVTDTIV (SEQ ID NO:59), DYCIPYNSVTDTIVV (SEQ ID NO:60), YCIPYNSVTDTIVVT (SEQ ID NO:61), GDGISTPKLKEDYQI (SEQ ID NO:62), DGISTPKLKEDYQIG (SEQ ID NO:63), GISTPKLKEDYQIGG (SEQ ID NO:64), ISTPKLKEDYQIGGY (SEQ ID NO:65), STPKLKEDYQIGGYS (SEQ ID NO:66), TPKLKEDYQIGGYSE (SEQ ID NO:67), PKLKEDYQIGGYSED (SEQ ID NO:68), KLKEDYQIGGYSEDR (SEQ ID NO:69), LKEDYQIGGYSEDRH (SEQ ID NO:70), KEDYQIGGYSEDRHS (SEQ ID NO:71), EDYQIGGYSEDRHSG (SEQ ID NO:72), DYQIGGYSEDRHSGV (SEQ ID NO:73), YQIGGYSEDRHSGVK (SEQ ID NO:74), QIGGYSEDRHSGVKD (SEQ ID NO:75), IGGYSEDRHSGVKDY (SEQ ID NO:76), GGYSEDRHSGVKDYV (SEQ ID NO:77), GYSEDRHSGVKDYVV (SEQ ID NO:78), YSEDRHSGVKDYVVV (SEQ ID NO:79), SEDRHSGVKDYVVVH (SEQ ID NO:80), EDRHSGVKDYVVVHG (SEQ ID NO:81), DRHSGVKDYVVVHGY (SEQ ID NO:82), RHSGVKDYVVVHGYF (SEQ ID NO:83), HSGVKDYVVVHGYFT (SEQ ID NO:84), SGVKDYVVVHGYFTE (SEQ ID NO:85), GVKDYVVVHGYFTEV (SEQ ID NO:86), ATFFIFNKLVKDPPN (SEQ ID NO:87), TFFIFNKLVKDPPNV (SEQ ID NO:88), FFIFNKLVKDPPNVQ (SEQ ID NO:89), FIFNKLVKDPPNVQI (SEQ ID NO:90), IFNKLVKDPPNVQIH (SEQ ID NO:91), FNKLVKDPPNVQIHT (SEQ ID NO:92), NKLVKDPPNVQIHTI (SEQ ID NO:93), KLVKDPPNVQIHTID (SEQ ID NO:94), LVKDPPNVQIHTIDG (SEQ ID NO:95), VKDPPNVQIHTIDGS (SEQ ID NO:96), KDPPNVQIHTIDGSS (SEQ ID NO:97), DGSSGVANPAMDPIY (SEQ ID NO:98), GSSGVANPAMDPIYD (SEQ ID NO:99), SSGVANPAMDPIYDE (SEQ ID NO:100), SGVANPAMDPIYDEP (SEQ ID NO:101), GVANPAMDPIYDEPT (SEQ ID NO:102), VANPAMDPIYDEPTT (SEQ ID NO:103), ANPAMDPIYDEPTTT (SEQ ID NO:104), NPAMDPIYDEPTTTT (SEQ ID NO:105), PAMDPIYDEPTTTTS (SEQ ID NO:106), AMDPIYDEPTTTTSV (SEQ ID NO:107), MDPIYDEPTTTTSVP (SEQ ID NO:108) and DPIYDEPTTTTSVPL (SEQ ID NO:109). These peptides are peptides of protein X1 from SARS-CoV Urbani. The above peptides having an amino acid sequence selected from the group consisting of INACRIIMRCWLCWK (SEQ ID NO:33), NACRIIMRCWLCWKC (SEQ ID NO:34), ACRIIMRCWLCWKCK (SEQ ID NO:35), CRIIMRCWLCWKCKS (SEQ ID NO:36), RIIMRCWLCWKCKSK (SEQ ID NO:37), IIMRCWLCWKCKSKN (SEQ ID NO:38), IMRCWLCWKCKSKNP (SEQ ID NO:39), MRCWLCWKCKSKNPL (SEQ ID NO:40), RCWLCWKCKSKNPLL (SEQ ID NO:41), CWLCWKCKSKNPLLY (SEQ ID NO:42), WLCWKCKSKNPLLYD (SEQ ID NO:43), LCWKCKSKNPLLYDA (SEQ ID NO:44) and CWKCKSKNPLLYDAN (SEQ ID NO:45) are peptides that are recognized in linear form. All of the other above peptides are recognized in linear as well as looped/cyclic form.

In another embodiment of the invention, the invention encompasses a peptide having an amino acid sequence selected from the group consisting of MMPTTLFAGTHITMT (SEQ ID NO:110), MPTTLFAGTHITMTT (SEQ ID NO:111), PTTLFAGTHITMTTV (SEQ ID NO:112), TTLFAGTHITMTTVY (SEQ ID NO:113), TLFAGTHITMTTVYH (SEQ ID NO:114), LFAGTHITMTTVYHI (SEQ ID NO:115), FAGTHITMTTVYHIT (SEQ ID NO:116), AGTHITMTTVYHITV (SEQ ID NO:117), GTHITMTTVYHITVS (SEQ ID NO:118), FQHQNSKKTTKLVVI (SEQ ID NO:119), QHQNSKKTTKLVVIL (SEQ ID NO:120), HQNSKKTTKLVVILR (SEQ ID NO:121), QNSKKTTKLVVILRI (SEQ ID NO:122), NSKKTTKLVVILRIG (SEQ ID NO:123), SKKTTKLVVILRIGT (SEQ ID NO:124), KKTTKLVVILRIGTQ (SEQ ID NO:125), KTTKLVVILRIGTQV (SEQ ID NO:126), TTKLVVILRIGTQVL (SEQ ID NO:127), TKLVVILRIGTQVLK (SEQ ID NO:128), KLVVILRIGTQVLKT (SEQ ID NO:129), LRIGTQVLKTMSLYM (SEQ ID NO:130), RIGTQVLKTMSLYMA (SEQ ID NO:131), IGTQVLKTMSLYMAI (SEQ ID NO:132), GTQVLKTMSLYMAIS (SEQ ID NO:133), TQVLKTMSLYMAISP (SEQ ID NO:134), QVLKTMSLYMAISPK (SEQ ID NO:135), VLKTMSLYMAISPKF (SEQ ID NO:136), LKTMSLYMAISPKFT (SEQ ID NO:137), KTMSLYMAISPKFTT (SEQ ID NO:138), MMSRRRLLACLCKHK (SEQ ID NO:139), MSRRRLLACLCKHKK (SEQ ID NO:140), SRRRLLACLCKHKKV (SEQ ID NO:141), RRRLLACLCKHKKVS (SEQ ID NO:142), RRLLACLCKHKKVST (SEQ ID NO:143), RLLACLCKHKKVSTN (SEQ ID NO:144), LLACLCKHKKVSTNL (SEQ ID NO:145), LACLCKHKKVSTNLC (SEQ ID NO:146), ACLCKHKKVSTNLCT (SEQ ID NO:147), CLCKHKKVSTNLCTH (SEQ ID NO:148), LCKHKKVSTNLCTHS (SEQ ID NO:149), CKHKKVSTNLCTHSF (SEQ ID NO:150), KHKKVSTNLCTHSFR (SEQ ID NO:151), HKKVSTNLCTHSFRK (SEQ ID NO:152), KKVSTNLCTHSFRKK (SEQ ID NO:153), KVSTNLCTHSFRKKQ (SEQ ID NO:154), VSTNLCTHSFRKKQV (SEQ ID NO:155) and STNLCTHSFRKKQVR (SEQ ID NO:156). These peptides are peptides of protein X2 from SARS-CoV Urbani. The above peptides having an amino acid sequence selected from the group consisting of MMSRRRLLACLCKHK (SEQ ID NO:139), MSRRRLLACLCKHKK (SEQ ID NO:140), SRRRLLACLCKHKKV (SEQ ID NO:141), RRRLLACLCKHKKVS (SEQ ID NO:142), RRLLACLCKHKKVST (SEQ ID NO:143), RLLACLCKHKKVSTN (SEQ ID NO:144), LLACLCKHKKVSTNL (SEQ ID NO:145), LACLCKHKKVSTNLC (SEQ ID NO:146), ACLCKHKKVSTNLCT (SEQ ID NO:147), CLCKHKKVSTNLCTH (SEQ ID NO:148), LCKHKKVSTNLCTHS (SEQ ID NO:149), CKHKKVSTNLCTHSF (SEQ ID NO:150), KHKKVSTNLCTHSFR (SEQ ID NO:151), HKKVSTNLCTHSFRK (SEQ ID NO:152), KKVSTNLCTHSFRKK (SEQ ID NO:153), KVSTNLCTHSFRKKQ (SEQ ID NO:154), VSTNLCTHSFRKKQV (SEQ ID NO:155) and STNLCTHSFRKKQVR (SEQ ID NO:156) are recognized in linear form. All of the other above peptides are recognized in linear as well as looped/cyclic form.

In another embodiment of the invention, the invention encompasses a peptide having an amino acid sequence from the group consisting of LCAYCCNIVNVSLVK (SEQ ID NO:157), CAYCCNIVNVSLVKP (SEQ ID NO:158), AYCCNIVNVSLVKPT (SEQ ID NO:159), YCCNIVNVSLVKPTV (SEQ ID NO:160), CCNIVNVSLVKPTVY (SEQ ID NO:161), CNIVNVSLVKPTVYV (SEQ ID NO:162), NIVNVSLVKPTVYVY (SEQ ID NO:163), IVNVSLVKPTVYVYS (SEQ ID NO:164), VNVSLVKPTVYVYSR (SEQ ID NO:165), NVSLVKPTVYVYSRV (SEQ ID NO:166), VSLVKPTVYVYSRVK (SEQ ID NO:167), SLVKPTVYVYSRVKN (SEQ ID NO:168), LVKPTVYVYSRVKNL (SEQ ID NO:169), VKPTVYVYSRVKNLN (SEQ ID NO:170), KPTVYVYSRVKNLNS (SEQ ID NO:171), PTVYVYSRVKNLNSS (SEQ ID NO:172), TVYVYSRVKNLNSSE (SEQ ID NO:173), VYVYSRVKNLNSSEG (SEQ ID NO:174), YVYSRVKNLNSSEGV (SEQ ID NO:175), VYSRVKNLNSSEGVP (SEQ ID NO:176), YSRVKNLNSSEGVPD (SEQ ID NO:177), SRVKNLNSSEGVPDL (SEQ ID NO:178), RVKNLNSSEGVPDLL (SEQ ID NO:179) and VKNLNSSEGVPDLLV (SEQ ID NO:180). These peptides are peptides of the E protein from SARS-CoV Urbani. All these peptides are recognized in linear as well as looped/cyclic form.

In another embodiment of the invention, the invention encompasses a peptide having an amino acid sequence selected from the group consisting of MADNGTITVEELKQL (SEQ ID NO:181), ADNGTITVEELKQLL (SEQ ID NO:182), DNGTITVEELKQLLE (SEQ ID NO:183), NGTITVEELKQLLEQ (SEQ ID NO:184), GTITVEELKQLLEQW (SEQ ID NO:185), TITVEELKQLLEQWN (SEQ ID NO:186), ITVEELKQLLEQWNL (SEQ ID NO:187), TVEELKQLLEQWNLV (SEQ ID NO:188), VEELKQLLEQWNLVI (SEQ ID NO:189), EELKQLLEQWNLVIG (SEQ ID NO:190), QFAYSNRNRFLYIIK (SEQ ID NO:191), FAYSNRNRFLYIIKL (SEQ ID NO:192), AYSNRNRFLYIIKLV (SEQ ID NO:193), YSNRNRFLYIIKLVF (SEQ ID NO:194), SNRNRFLYIIKLVFL (SEQ ID NO:195), NRNRFLYIIKLVFLW (SEQ ID NO:196), RNRFLYIIKLVFLWL (SEQ ID NO:197), NRFLYIIKLVFLWLL (SEQ ID NO:198), RFLYIIKLVFLWLLW (SEQ ID NO:199), FLYIIKLVFLWLLWP (SEQ ID NO:200), INWVTGGIAIAMACI (SEQ ID NO:201), NWVTGGIAIAMACIV (SEQ ID NO:202), WVTGGIAIAMACIVG (SEQ ID NO:203), VTGGIAIAMACIVGL (SEQ ID NO:204), TGGIAIAMACIVGLM (SEQ ID NO:205), GGIAIAMACIVGLMW (SEQ ID NO:206), GIAIAMACIVGLMWL (SEQ ID NO:207), IAIAMACIVGLMWLS (SEQ ID NO:208), LMWLSYFVASFRLFA (SEQ ID NO:209), MWLSYFVASFRLFAR (SEQ ID NO:210), WLSYFVASFRLFART (SEQ ID NO:211), LSYFVASFRLFARTR (SEQ ID NO:212), SYFVASFRLFARTRS (SEQ ID NO:213), YFVASFRLFARTRSM (SEQ ID NO:214), FVASFRLFARTRSMW (SEQ ID NO:215), VASFRLFARTRSMWS (SEQ ID NO:216), NILLNVPLRGTIVTR (SEQ ID NO:217), ILLNVPLRGTIVTRP (SEQ ID NO:218), LLNVPLRGTIVTRPL (SEQ ID NO:219), LNVPLRGTIVTRPLM (SEQ ID NO:220), NVPLRGTIVTRPLME (SEQ ID NO:221), VPLRGTIVTRPLMES (SEQ ID NO:222), PLRGTIVTRPLMESE (SEQ ID NO:223), LRGTIVTRPLMESEL (SEQ ID NO:224), RGTIVTRPLMESELV (SEQ ID NO:225), GTIVTRPLMESELVI (SEQ ID NO:226), TIVTRPLMESELVIG (SEQ ID NO:227), IVTRPLMESELVIGA (SEQ ID NO:229), VTRPLMESELVIGAV (SEQ ID NO:230), TRPLMESELVIGAVI (SEQ ID NO:231), RPLMESELVIGAVII (SEQ ID NO:232), VIGAVIIRGHLRMAG (SEQ ID NO:233), IGAVIIRGHLRMAGH (SEQ ID NO:234), GAVIIRGHLRMAGHP (SEQ ID NO:235), AVIIRGHLRMAGHPL (SEQ ID NO:236), VIIRGHLRMAGHPLG (SEQ ID NO:237), IIRGHLRMAGHPLGR (SEQ ID NO:238), IRGHLRMAGHPLGRC (SEQ ID NO:239), RGHLRMAGHPLGRCD (SEQ ID NO:240), GHLRMAGHPLGRCDI (SEQ ID NO:241), HLRMAGHPLGRCDIK (SEQ ID NO:242), LRMAGHPLGRCDIKD (SEQ ID NO:243), RMAGHPLGRCDIKDL (SEQ ID NO:244), MAGHPLGRCDIKDLP (SEQ ID NO:245), AGHPLGRCDIKDLPK (SEQ ID NO:246), GHPLGRCDIKDLPKE (SEQ ID NO:247), HPLGRCDIKDLPKEI (SEQ ID NO:248), PLGRCDIKDLPKEIT (SEQ ID NO:249), LGRCDIKDLPKEITV (SEQ ID NO:250), GRCDIKDLPKEITVA (SEQ ID NO:251), TLSYYKLGASQRVGT (SEQ ID NO:252), LSYYKLGASQRVGTD (SEQ ID NO:253), SYYKLGASQRVGTDS (SEQ ID NO:254), YYKLGASQRVGTDSG (SEQ ID NO:255), YKLGASQRVGTDSGF (SEQ ID NO:256), KLGASQRVGTDSGFA (SEQ ID NO:257), LGASQRVGTDSGFAA (SEQ ID NO:258), GASQRVGTDSGFAAY (SEQ ID NO:259), ASQRVGTDSGFAAYN (SEQ ID NO:260), IGNYKLNTDHAGSND (SEQ ID NO:261), GNYKLNTDHAGSNDN (SEQ ID NO:262), NYKLNTDHAGSNDNI (SEQ ID NO:263), YKLNTDHAGSNDNIA (SEQ ID NO:264), KLNTDHAGSNDNIAL (SEQ ID NO:265), LNTDHAGSNDNIALL (SEQ ID NO:266), NTDHAGSNDNIALLV (SEQ ID NO:267) and TDHAGSNDNIALLVQ (SEQ ID NO:268). These peptides are peptides of the M protein from SARS-CoV Urbani. The above peptides having an amino acid sequence selected from the group consisting of QFAYSNRNRFLYIIK (SEQ ID NO:191), FAYSNRNRFLYIIKL (SEQ ID NO:192), AYSNRNRFLYIIKLV (SEQ ID NO:193), YSNRNRFLYIIKLVF (SEQ ID NO:194), SNRNRFLYIIKLVFL (SEQ ID NO:195), NRNRFLYIIKLVFLW (SEQ ID NO:196), RNRFLYIIKLVFLWL (SEQ ID NO:197), NRFLYIIKLVFLWLL (SEQ ID NO:198), RFLYIIKLVFLWLLW (SEQ ID NO:199), FLYIIKLVFLWLLWP (SEQ ID NO:200), LMWLSYFVASFRLFA (SEQ ID NO:209), MWLSYFVASFRLFAR (SEQ ID NO:210), WLSYFVASFRLFART (SEQ ID NO:211), LSYFVASFRLFARTR (SEQ ID NO:212), SYFVASFRLFARTRS (SEQ ID NO:213), YFVASFRLFARTRSM (SEQ ID NO:214), FVASFRLFARTRSMW (SEQ ID NO:215), VASFRLFARTRSMWS (SEQ ID NO:216), NILLNVPLRGTIVTR (SEQ ID NO:217), ILLNVPLRGTIVTRP (SEQ ID NO:218), LLNVPLRGTIVTRPL (SEQ ID NO:219), LNVPLRGTIVTRPLM (SEQ ID NO:220), NVPLRGTIVTRPLME (SEQ ID NO:221), VPLRGTIVTRPLMES (SEQ ID NO:222), PLRGTIVTRPLMESE (SEQ ID NO:223), LRGTIVTRPLMESEL (SEQ ID NO:224), RGTIVTRPLMESELV (SEQ ID NO:225), GTIVTRPLMESELVI (SEQ ID NO:226), TIVTRPLMESELVIG (SEQ ID NO:227), IVTRPLMESELVIGA (SEQ ID NO:229), VTRPLMESELVIGAV (SEQ ID NO:230), TRPLMESELVIGAVI (SEQ ID NO:231) and RPLMESELVIGAVII (SEQ ID NO:232) are recognized in looped/cyclic form. All of the other above peptides are recognized in linear as well as looped/cyclic form.

In another embodiment of the invention, the invention encompasses a peptide having an amino acid sequence selected from the group consisting of AEILIIIMRTFRIAI (SEQ ID NO:269), EILIIIMRTFRIAIW (SEQ ID NO:270), ILIIIMRTFRIAIWN (SEQ ID NO:271), LIIIMRTFRIAIWNL (SEQ ID NO:272), IIIMRTFRIAIWNLD (SEQ ID NO:273), IIMRTFRIAIWNLDV (SEQ ID NO:274), IMRTFRIAIWNLDVI (SEQ ID NO:275), MRTFRIAIWNLDVII (SEQ ID NO:276), RTFRIAIWNLDVIIS (SEQ ID NO:277), VIISSIVRQLFKPLT (SEQ ID NO:278), IISSIVRQLFKPLTK (SEQ ID NO:279), ISSIVRQLFKPLTKK (SEQ ID NO:280), SSIVRQLFKPLTKKN (SEQ ID NO:281), SIVRQLFKPLTKKNY (SEQ ID NO:282), IVRQLFKPLTKKNYS (SEQ ID NO:283), VRQLFKPLTKKNYSE (SEQ ID NO:284), RQLFKPLTKKNYSEL (SEQ ID NO:285), QLFKPLTKKNYSELD (SEQ ID NO:286), LFKPLTKKNYSELDD (SEQ ID NO:287), FKPLTKKNYSELDDE (SEQ ID NO:288), KPLTKKNYSELDDEE (SEQ ID NO:289), PLTKKNYSELDDEEP (SEQ ID NO:290), LTKKNYSELDDEEPM (SEQ ID NO:291), TKKNYSELDDEEPME (SEQ ID NO:292), KKNYSELDDEEPMEL (SEQ ID NO:293), KNYSELDDEEPMELD (SEQ ID NO:294), NYSELDDEEPMELDY (SEQ ID NO:295) and YSELDDEEPMELDYP (SEQ ID NO:296). These peptides are peptides of the protein X3 from SARS-CoV Urbani. All of the above peptides are recognized in linear and looped/cyclic form.

In another embodiment, the invention encompasses a peptide having an amino acid sequence selected from the group consisting of ELYHYQECVRGTTVL (SEQ ID NO:297), LYHYQECVRGTTVLL (SEQ ID NO:298), YHYQECVRGTTVLLK (SEQ ID NO:299), HYQECVRGTTVLLKE (SEQ ID NO:300), YQECVRGTTVLLKEP (SEQ ID NO:301), QECVRGTTVLLKEPC (SEQ ID NO:302), ECVRGTTVLLKEPCP (SEQ ID NO:303), CVRGTTVLLKEPCPS (SEQ ID NO:304), VRGTTVLLKEPCPSG (SEQ ID NO:305), RGTTVLLKEPCPSGT (SEQ ID NO:306), GTTVLLKEPCPSGTY (SEQ ID NO:307), TTVLLKEPCPSGTYE (SEQ ID NO:308), TVLLKEPCPSGTYEG (SEQ ID NO:309), CPSGTYEGNSPFHPL (SEQ ID NO:310), PSGTYEGNSPFHPLA (SEQ ID NO:311), SGTYEGNSPFHPLAD (SEQ ID NO:312), GTYEGNSPFHPLADN (SEQ ID NO:313), TYEGNSPFHPLADNK (SEQ ID NO:314), YEGNSPFHPLADNKF (SEQ ID NO:315), EGNSPFHPLADNKFA (SEQ ID NO:316), GNSPFHPLADNKFAL (SEQ ID NO:317), NSPFHPLADNKFALT (SEQ ID NO:318), SPFHPLADNKFALTC (SEQ ID NO:319), PFHPLADNKFALTCT (SEQ ID NO:320), FHPLADNKFALTCTS (SEQ ID NO:321), HPLADNKFALTCTST (SEQ ID NO:322), PLADNKFALTCTSTH (SEQ ID NO:323), LADNKFALTCTSTHF (SEQ ID NO:324), ADNKFALTCTSTHFA (SEQ ID NO:325), DNKFALTCTSTHFAF (SEQ ID NO:326), FIRQEEVQQELYSPL (SEQ ID NO:327), IRQEEVQQELYSPLF (SEQ ID NO:328), RQEEVQQELYSPLFL (SEQ ID NO:329), QEEVQQELYSPLFLI (SEQ ID NO:330), EEVQQELYSPLFLIV (SEQ ID NO:331), EVQQELYSPLFLIVA (SEQ ID NO:332) and VQQELYSPLFLIVAA (SEQ ID NO:333). These peptides are peptides of protein X4 from SARS-CoV Urbani. The above peptides having an amino acid sequence selected from the group consisting of FIRQEEVQQELYSPL (SEQ ID NO:327), IRQEEVQQELYSPLF (SEQ ID NO:328), RQEEVQQELYSPLFL (SEQ ID NO:329), QEEVQQELYSPLFLI (SEQ ID NO:330), EEVQQELYSPLFLIV (SEQ ID NO:331), EVQQELYSPLFLIVA (SEQ ID NO:332) and VQQELYSPLFLIVAA (SEQ ID NO:333) are recognized in looped/cyclic form, while all other of the above peptides are recognized in linear and looped/cyclic form.

In another embodiment, the invention encompasses a peptide having an amino acid sequence selected from the group consisting of RWHTMVQTCTPNVTI (SEQ ID NO:334), WHTMVQTCTPNVTIN (SEQ ID NO:335), HTMVQTCTPNVTINC (SEQ ID NO:336), TMVQTCTPNVTINCQ (SEQ ID NO:337), MVQTCTPNVTINCQD (SEQ ID NO:338), PNVTINCQDPAGGAL (SEQ ID NO:339), NVTINCQDPAGGALI (SEQ ID NO:340), VTINCQDPAGGALIA (SEQ ID NO:341), TINCQDPAGGALIAR (SEQ ID NO:342), INCQDPAGGALIARC (SEQ ID NO:343), NCQDPAGGALIARCW (SEQ ID NO:344), CQDPAGGALIARCWY (SEQ ID NO:345), QDPAGGALIARCWYL (SEQ ID NO:346), IARCWYLHEGHQTAA (SEQ ID NO:347), ARCWYLHEGHQTAAF (SEQ ID NO:348), RCWYLHEGHQTAAFR (SEQ ID NO:349), CWYLHEGHQTAAFRD (SEQ ID NO:350), WYLHEGHQTAAFRDV (SEQ ID NO:351), YLHEGHQTAAFRDVL (SEQ ID NO:352), LHEGHQTAAFRDVLV (SEQ ID NO:353), HEGHQTAAFRDVLVV (SEQ ID NO:354), EGHQTAAFRDVLVVL (SEQ ID NO:355), GHQTAAFRDVLVVLN (SEQ ID NO:356) and HQTAAFRDVLVVLNK (SEQ ID NO:357). These peptides are peptides of protein X5 from SARS-CoV Urbani. All of these peptides are recognized in linear as well as looped/cyclic form.

In another embodiment of the invention, the peptide has an amino acid sequence selected from the group consisting of NNAATVLQLPQGTTL (SEQ ID NO:358), NAATVLQLPQGTTLP (SEQ ID NO:359), AATVLQLPQGTTLPK (SEQ ID NO:360), ATVLQLPQGTTLPKG (SEQ ID NO:361), TVLQLPQGTTLPKGF (SEQ ID NO:362), VLQLPQGTTLPKGFY (SEQ ID NO:363), LQLPQGTTLPKGFYA (SEQ ID NO:364), QLPQGTTLPKGFYAE (SEQ ID NO:365), LPQGTTLPKGFYAEG (SEQ ID NO:366), PQGTTLPKGFYAEGS (SEQ ID NO:367), QGTTLPKGFYAEGSR (SEQ ID NO:368), GTTLPKGFYAEGSRG (SEQ ID NO:369), TTLPKGFYAEGSRGG (SEQ ID NO:370), TLPKGFYAEGSRGGS (SEQ ID NO:371), NSPARMASGGGETAL (SEQ ID NO:372), SPARMASGGGETALA (SEQ ID NO:373), PARMASGGGETALAL (SEQ ID NO:374), ARMASGGGETALALL (SEQ ID NO:375), RMASGGGETALALLL (SEQ ID NO:376), MASGGGETALALLLL (SEQ ID NO:377), ASGGGETALALLLLD (SEQ ID NO:378), QQGQTVTKKSAAEAS (SEQ ID NO:379), QGQTVTKKSAAEASK (SEQ ID NO:380), GQTVTKKSAAEASKK (SEQ ID NO:381), QTVTKKSAAEASKKP (SEQ ID NO:382), TVTKKSAAEASKKPR (SEQ ID NO:383), VTKKSAAEASKKPRQ (SEQ ID NO:384), TKKSAAEASKKPRQK (SEQ ID NO:385), KKSAAEASKKPRQKR (SEQ ID NO:386), KSAAEASKKPRQKRT (SEQ ID NO:387), SAAEASKKPRQKRTA (SEQ ID NO:388), AAEASKKPRQKRTAT (SEQ ID NO:389), KPRQKRTATKQYNVT (SEQ ID NO:390), PRQKRTATKQYNVTQ (SEQ ID NO:391), RQKRTATKQYNVTQA (SEQ ID NO:392), QKRTATKQYNVTQAF (SEQ ID NO:393), KRTATKQYNVTQAFG (SEQ ID NO:394), RTATKQYNVTQAFGR (SEQ ID NO:395), TATKQYNVTQAFGRR (SEQ ID NO:396), FGRRGPEQTQGNFGD (SEQ ID NO:397), GRRGPEQTQGNFGDQ (SEQ ID NO:398), RRGPEQTQGNFGDQD (SEQ ID NO:399), RGPEQTQGNFGDQDL (SEQ ID NO:400), GPEQTQGNFGDQDLI (SEQ ID NO:401), PEQTQGNFGDQDLIR (SEQ ID NO:402), EQTQGNFGDQDLIRQ (SEQ ID NO:403), QTQGNFGDQDLIRQG (SEQ ID NO:404), IKLDDKDPQFKDNVI (SEQ ID NO:405), KLDDKDPQFKDNVIL (SEQ ID NO:406), LDDKDPQFKDNVILL (SEQ ID NO:407), DDKDPQFKDNVILLN (SEQ ID NO:408), DKDPQFKDNVILLNK (SEQ ID NO:409), KDPQFKDNVILLNKH (SEQ ID NO:410), DPQFKDNVILLNKHI (SEQ ID NO:411), PQFKDNVILLNKHID (SEQ ID NO:412), QFKDNVILLNKHIDA (SEQ ID NO:413), QPLPQRQKKQPTVTL (SEQ ID NO:414), PLPQRQKKQPTVTLL (SEQ ID NO:415), LPQRQKKQPTVTLLP (SEQ ID NO:416), PQRQKKQPTVTLLPA (SEQ ID NO:417), QRQKKQPTVTLLPAA (SEQ ID NO:418), RQKKQPTVTLLPAAD (SEQ ID NO:419) and QKKQPTVTLLPAADM (SEQ ID NO:420). These peptides are peptides of the N protein from SARS-CoV Urbani. The above peptides having an amino acid sequence selected from the group consisting of QQGQTVTKKSAAEAS (SEQ ID NO:379), QGQTVTKKSAAEASK (SEQ ID NO:380), GQTVTKKSAAEASKK (SEQ ID NO:381), QTVTKKSAAEASKKP (SEQ ID NO:382), TVTKKSAAEASKKPR (SEQ ID NO:383), VTKKSAAEASKKPRQ (SEQ ID NO:384), TKKSAAEASKKPRQK (SEQ ID NO:385), KKSAAEASKKPRQKR (SEQ ID NO:386), KSAAEASKKPRQKRT (SEQ ID NO:387), SAAEASKKPRQKRTA (SEQ ID NO:388), AAEASKKPRQKRTAT (SEQ ID NO:389), FGRRGPEQTQGNFGD (SEQ ID NO:397), GRRGPEQTQGNFGDQ (SEQ ID NO:398), RRGPEQTQGNFGDQD (SEQ ID NO:399), RGPEQTQGNFGDQDL (SEQ ID NO:400), GPEQTQGNFGDQDLI (SEQ ID NO:401), PEQTQGNFGDQDLIR (SEQ ID NO:402), EQTQGNFGDQDLIRQ (SEQ ID NO:403) and QTQGNFGDQDLIRQG (SEQ ID NO:404) are recognized in linear form. The above peptides having an amino acid sequence selected from the group consisting of QPLPQRQKKQPTVTL (SEQ ID NO:414), PLPQRQKKQPTVTLL (SEQ ID NO:415), LPQRQKKQPTVTLLP (SEQ ID NO:416), PQRQKKQPTVTLLPA (SEQ ID NO:417), QRQKKQPTVTLLPAA (SEQ ID NO:418), RQKKQPTVTLLPAAD (SEQ ID NO:419) and QKKQPTVTLLPAADM (SEQ ID NO:420) are recognized in looped/cyclic form. All of the other above peptides are recognized in linear and looped/cyclic form. A particularly interesting region due to its high reactivity with several sera is the region of the N protein containing the continuous series of linear and/or looped peptides starting with the sequence AATVLQLPQGTTLPK (SEQ ID NO:360) and ending with the peptide QGTTLPKGFYAEGSR (SEQ ID NO:368), thereby having the minimal sequence QGTTLPK (SEQ ID NO:606) in common.

All the oligopeptides identified above are good candidates to represent a neutralizing epitope of SARS-CoV, particularly SARS-CoV Urbani and/or other strains comprising the above oligopeptides. They may be used in therapy and/or prevention of conditions resulting from an infection with SARS-CoV as described herein and may also be used in diagnostic test methods as described herein.

In a further aspect of the invention, peptides mentioned above may be coupled/linked to each other. Peptides of the embodiments of the invention may be coupled/linked to peptides of other embodiments of the invention or the same embodiment of the invention. The peptides may be linear and/or looped/cyclic. A combination peptide may also constitute of more than two peptides. The peptides of the invention can be linked directly or indirectly via for instance a spacer of variable length. Furthermore, the peptides can be linked covalently or non-covalently. They may also be part of a fusion protein or conjugate.

A combination peptide which contains different peptides from one embodiment of the invention, i.e. from one protein, may mimic/simulate a discontinuous and/or conformational epitope. Such an epitope may be more antigenic than the single peptides. In general, the peptides should be in such a form as to be capable of mimicking/simulating a discontinuous and/or conformational epitope.

Obviously, the person skilled in the art may make modifications to the peptide without departing from the scope of the invention, e.g. by systematic length variation and/or replacement of residues and/or combination with other peptides. Peptides can be synthesized by known solid phase peptide synthesis techniques. The synthesis allows for one or more amino acids not corresponding to the original peptide sequence to be added to the amino or carboxyl terminus of the peptides. Such extra amino acids are useful for coupling the peptides to each other, to another peptide, to a large carrier protein or to a solid support. Amino acids that are inter alia useful for these purposes include tyrosine, lysine, glutamic acid, aspartic acid, cysteine and derivatives thereof. Additional protein modification techniques may be used, e.g., NH₂-acetylation or COOH-terminal amidation, to provide additional means for coupling the peptides to another protein or peptide molecule or to a support, for example, polystyrene or polyvinyl microtiter plates, glass tubes or glass beads or particles and chromatographic supports, such as paper, cellulose and cellulose derivates, and silica. If the peptide is coupled to such a support, it may also be used for affinity purification of SARS-CoV recognizing antibodies.

In an embodiment the peptides of the invention can have a looped/cyclic form. Linear peptides can be made by chemically converting the structures to looped/cyclic forms. It is well known in the art that cyclization of linear peptides can modulate bioactivity by increasing or decreasing the potency of binding to the target protein. Linear peptides are very flexible and tend to adopt many different conformations in solution. Cyclization acts to constrain the number of available conformations, and thus, favor the more active or inactive structures of the peptide. Cyclization of linear peptides is accomplished either by forming a peptide bond between the free N-terminal and C-terminal ends (homodetic cyclopeptides) or by forming a new covalent bond between amino acid backbone and/or side chain groups located near the N- or C-terminal ends (heterodetic cyclopeptides). The latter cyclizations use alternate chemical strategies to form covalent bonds, for example, disulfides, lactones, ethers, or thioethers. However, cyclization methods other than the ones described above can also be used to form cyclic/looped peptides. Generally, linear peptides of more than five residues can be cyclized relatively easily. The propensity of the peptide to form a beta-turn conformation in the central four residues facilitates the formation of both homo- and heterodetic cyclopeptides. The looped/cyclic peptides of the invention preferably comprise a cysteine residue at position 2 and 14. Preferably, they contain a linker between the cysteine residues. The looped/cyclic peptides of the invention are recognized by antibodies in the serum of individuals that have been and/or are infected with SARS-CoV.

Alternatively, the peptides of the invention may be prepared by expression of the peptides or of a larger peptide including the desired peptide from a corresponding gene (whether synthetic or natural in origin) in a suitable host. The larger peptide may contain a cleavage site whereby the peptide of interest may be released by cleavage of the fused molecule.

The resulting peptides may then be tested for binding to sera from subjects that have been previously infected with SARS-CoV, to sera from infected subjects or to purified (recombinant) SARS-CoV antibodies in a way essentially as described herein. If such a peptide can still be bound by the sera or antibody, it is considered as a functional fragment or analogue of the peptides according to the invention. Also, even stronger antigenic peptides may be identified in this manner, which peptides may be used for vaccination purposes or for generating strongly neutralizing antibodies for therapeutic and/or prophylactic purposes. The peptides may also be used in diagnostic tests. Therefore the invention also provides the peptides comprising a part (or even consisting of a part) of a peptide according to the invention, wherein said part is recognized by antibodies present in serum derived from a subject/individual that has been and/or is infected by SARS-CoV or wherein said part is recognized by a recombinant monoclonal antibody such as the antibody 03-018.

Furthermore, the invention provides peptides consisting of an analogue of a peptide according to the invention, wherein one or more amino acids are substituted for another amino acid, and wherein said analogue is recognized by antibodies present in serum derived from a subject/individual that has been and/or is infected by SARS-CoV or wherein said part is recognized by a recombinant monoclonal antibody such as the antibody 03-018. Alternatively, further embodiments comprise analogues of the various embodiments of the present invention comprising an amino acid sequence containing insertions, deletions or combinations thereof of one or more amino acids compared to the amino acid sequences of the parent peptides. Furthermore, analogues can comprise truncations of the amino acid sequence at either or both the amino or carboxy termini of the peptides. Analogues according to the invention may have the same or different, either higher or lower, antigenic properties compared to the parent peptides, but are still recognized by antibodies present in serum derived from an individual that has been and/or is infected by SARS-CoV or by a recombinant monoclonal antibody such as the antibody 03-018. That part of a 15-mer still representing immunogenic activity consists of about 6-12, preferably 7-11, more preferably 8-10, even more preferably 9 amino acids within the 15-mer.

The peptides, parts thereof or analogues thereof according to the invention may be used directly as peptides, but may also be used conjugated to an immunogenic carrier, which may be, e.g. a polypeptide or polysaccharide. If the carrier is a polypeptide, the desired conjugate may be expressed as a fusion protein. Alternatively, the peptide and the carrier may be obtained separately and then conjugated. This conjugation may be covalently or non-covalently. A fusion protein is a chimeric protein, comprising the peptide according to the invention, and another protein or part thereof not being a SARS-CoV protein. Such fusion proteins may for instance be used to raise antibodies for diagnostic, prophylactic or therapeutic purposes or to directly immunize, i.e. vaccinate, humans or animals. Any protein or part thereof or even peptide may be used as fusion partner for the peptide according to the invention to form a fusion protein, and non-limiting examples are bovine serum albumin, keyhole limpet hemocyanin, etc.

The peptides may be labeled (signal-generating) or unlabeled. This depends on the type of assay used. Labels which may be coupled to the peptides are those known in the art and include, but are not limited to, enzymes, radionuclides, fluorogenic and chromogenic substrates, cofactors, biotin/avidin, colloidal gold, and magnetic particles.

It is another aspect of the invention to provide nucleic acid molecules encoding peptides, parts thereof or analogues thereof or fusion proteins according to the invention. Such nucleic acid molecules may suitably be used in the form of plasmids for propagation and expansion in bacterial or other hosts. Moreover, recombinant DNA techniques well known to the person skilled in the art can be used to obtain nucleic acid molecules encoding analogues of the peptides according to the invention, e.g. by mutagenesis of the sequences encoding the peptides according to the invention. The skilled man will appreciate that analogues of the nucleic acid molecules are also intended to be a part of the present invention. Analogues are also nucleic acid sequences that can be directly translated, using the standard genetic code, to provide an amino acid sequence identical to that translated from the parent nucleic acid molecules. Another aspect of nucleic acid molecules according to the present invention, is their potential for use in gene-therapy or vaccination applications. Therefore, in another embodiment of the invention, nucleic acid molecules according to the invention are provided wherein said nucleic acid molecule is present in a gene delivery vehicle. A “gene delivery vehicle” as used herein refers to an entity that can be used to introduce nucleic acid molecules into cells, and includes liposomes, naked DNA, plasmid DNA, optionally coupled to a targeting moiety such as an antibody with specificity for an antigen presenting cell, recombinant viruses, and the like. Preferred gene therapy vehicles of the present invention will generally be viral vectors, such as comprised within a recombinant retrovirus, herpes simplex virus (HSV), adenovirus, adeno-associated virus (AAV), cytomegalovirus (CMV), and the like. Such applications of the nucleic acid sequences according to the invention are included in the present invention. The person skilled in the art will be aware of the possibilities of recombinant viruses for administering sequences of interest to cells. The administration of the nucleic acids of the invention to cells can result in an enhanced immune response. Alternatively, the nucleic acid encoding the peptides of the invention can be used as naked DNA vaccines, e.g. immunization by injection of purified nucleic acid molecules into humans or animals.

In another aspect, the invention provides antibodies recognizing the peptides, parts or analogues thereof of the invention. Antibodies can be obtained according to routine methods well known to the person skilled in the art, including but not limited to immunization of animals such as mice, rabbits, goats, and the like, or by antibody, phage or ribosome display methods (see e.g. Using Antibodies: A Laboratory Manual, Edited by: E. Harlow, D. Lane (1998), Cold Spring Harbor Laboratory Press, Cold Spring Harbor, N.Y.; Current Protocols in Immunology, Edited by: J. E. Coligan, A. M. Kruisbeek, D. H. Margulies, E. M. Shevach, W. Strober (2001), John Wiley & Sons Inc., New York; and Phage Display: A Laboratory Manual. Edited by: C. F. Barbas, D. R. Burton, J. K. Scott and G. J. Silverman (2001), Cold Spring Harbor Laboratory Press, Cold Spring Harbor, N.Y., the disclosures of which are incorporated herein by reference).

The antibodies of the invention can be intact immunoglobulin molecules such as polyclonal or monoclonal antibodies, in particular human monoclonal antibodies, or the antibodies can be functional fragments thereof, i.e. fragments that are still capable of binding to the antigen. These fragments include, but not limited to, Fab, F(ab′), F(ab′)₂, Fv, dAb, Fd, complementarity determining region (CDR) fragments, single-chain antibodies (scFv), bivalent single-chain antibodies, diabodies, triabodies, tetrabodies, and (poly)peptides that contain at least a fragment of an immunoglobulin that is sufficient to confer specific antigen binding to the (poly)peptides. The antibodies of the invention can be used in non-isolated or isolated form. Furthermore, the antibodies of the invention can be used alone or in a mixture/composition comprising at least one antibody (or variant or fragment thereof) of the invention. Antibodies of the invention include all the immunoglobulin classes and subclasses known in the art. Depending on the amino acid sequence of the constant domain of their heavy chains, binding molecules can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4. The above mentioned antigen-binding fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D. Lane (1988), Cold Spring Harbor Laboratory Press, Cold Spring Harbor, N.Y., which is incorporated herein by reference. A binding molecule or antigen-binding fragment thereof may have one or more binding sites. If there is more than one binding site, the binding sites may be identical to one another or they may be different.

The antibodies of the invention can be naked or unconjugated antibodies. A naked or unconjugated antibody is intended to refer to an antibody that is not conjugated, operatively linked or otherwise physically or functionally associated with an effector moiety or tag, such as inter alia a toxic substance, a radioactive substance, a liposome, an enzyme. It will be understood that naked or unconjugated antibodies do not exclude antibodies that have been stabilized, multimerized, humanized or in any other way manipulated, other than by the attachment of an effector moiety or tag. Accordingly, all post-translationally modified naked and unconjugated antibodies are included herewith, including where the modifications are made in the natural antibody-producing cell environment, by a recombinant antibody-producing cell, and are introduced by the hand of man after initial antibody preparation. Of course, the term naked or unconjugated antibody does not exclude the ability of the antibody to form functional associations with effector cells and/or molecules after administration to the body, as some of such interactions are necessary in order to exert a biological effect. The lack of associated effector group or tag is therefore applied in definition to the naked or unconjugated binding molecule in vitro, not in vivo.

Alternatively, the antibodies as described in the present invention can be conjugated to tags and be used for detection and/or analytical and/or diagnostic purposes. The tags used to label the antibodies for those purposes depend on the specific detection/analysis/diagnosis techniques and/or methods used such as inter alia immunohistochemical staining of tissue samples, flow cytometric detection, scanning laser cytometric detection, fluorescent immunoassays, enzyme-linked immunosorbent assays (ELISAs), radioimmunoassays (RIAs), bioassays (e.g., neutralization assays, growth inhibition assays), Western blotting applications, etc. For immunohistochemical staining of tissue samples preferred labels are enzymes that catalyze production and local deposition of a detectable product. Enzymes typically conjugated to antibodies to permit their immunohistochemical visualization are well-known and include, but are not limited to, alkaline phosphatase, P-galactosidase, glucose oxidase, horseradish peroxidase, and urease. Typical substrates for production and deposition of visually detectable products include, but are not limited to, o-nitrophenyl-beta-D-galactopyranoside (ONPG), o-phenylenediamine dihydrochloride (OPD), p-nitrophenyl phosphate (PNPP), p-nitrophenyl-beta-D-galactopryanoside (PNPG), 3′,3′diaminobenzidine (DAB), 3-amino-9-ethylcarbazole (AEC), 4-chloro-1-naphthol (CN), 5-bromo-4-chloro-3-indolyl-phosphate (BCIP), ABTS, BluoGal, iodonitrotetrazolium (INT), nitroblue tetrazolium chloride (NBT), phenazine methosulfate (PMS), phenolphthalein monophosphate (PMP), tetramethyl benzidine (TMB), tetranitroblue tetrazolium (TNBT), X-Gal, X-Gluc, and X-glucoside. Other substrates that can be used to produce products for local deposition are luminescent substrates. For example, in the presence of hydrogen peroxide, horseradish peroxidase can catalyze the oxidation of cyclic diacylhydrazides such as luminol. Next to that, binding molecules of the immunoconjugate of the invention can also be labeled using colloidal gold or they can be labeled with radioisotopes, such as ³³p, ³²p, ³⁵S, ³H, and ¹²⁵I. When the antibodies of the present invention are used for flow cytometric detections, scanning laser cytometric detections, or fluorescent immunoassays, they can usefully be labeled with fluorophores. A wide variety of fluorophores useful for fluorescently labeling the antibodies of the present invention include, but are not limited to, Alexa Fluor and Alexa Fluor&commat dyes, BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Cy2, Cy3, Cy3.5, Cy5, Cy5.5, Cy7, fluorescein isothiocyanate (FITC), allophycocyanin (APC), R-phycoerythrin (PE), peridinin chlorophyll protein (PerCP), Texas Red, fluorescence resonance energy tandem fluorophores such as PerCP-Cy5.5, PE-Cy5, PE-Cy5.5, PE-Cy7, PE-Texas Red, and APC-Cy7. When the antibodies of the present invention are used for secondary detection using labeled avidin, streptavidin, captavidin or neutravidin, the antibodies may be labeled with biotin.

Next to that, the antibodies of the invention may be conjugated to photoactive agents or dyes such as fluorescent and other chromogens or dyes to use the so obtained immunoconjugates in photoradiation, phototherapy, or photodynamic therapy. The photoactive agents or dyes include, but are not limited to, photofrin.RTM, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

When the antibodies of the invention are used for in vivo diagnostic use, the antibodies can also be made detectable by conjugation to e.g. magnetic resonance imaging (MRI) contrast agents, such as gadolinium diethylenetriaminepentaacetic acid, to ultrasound contrast agents or to X-ray contrast agents, or by radioisotopic labeling.

The antibodies according to the invention may be capable of neutralizing SARS-CoV infectivity and are useful for therapeutic purposes against this virus. Assays to detect and measure virus neutralizing activity of antibodies are well known in the art. For example, a SARS-CoV neutralization assay can be performed on Vero cells (ATCC CCL 81). Antibodies of the invention are mixed with virus suspension and incubated for one hour at 37° C. The obtained suspension is then inoculated onto sub-confluent Vero cells (approximately 80% density) grown in 96-well cell-culture plates. The inoculated cells are cultured for 3-4 days at 37° C. and observed daily for the development of cytopathic effect (CPE). CPE is compared to the positive control (virus inoculated cells) and negative controls (mock-inoculated cells or cells incubated with antibody only). Alternatively, the antibodies may inhibit or down-regulate SARS-CoV replication, are complement fixing antibodies capable of assisting in the lysis of enveloped SARS-CoV and/or act as opsonins and augment phagocytosis of SARS-CoV either by promoting its uptake via Fc or C3b receptors or by agglutinating SARS-CoV to make it more easily phagocytosed.

The invention also provides nucleic acid molecules encoding the antibodies according to the invention.

It is another aspect of the invention to provide vectors, i.e. nucleic acid constructs, comprising one or more nucleic acid molecules according to the present invention. The nucleic acid molecule may either encode the peptides, parts or analogues thereof or fusion proteins of the invention or encode the antibodies of the invention. Vectors can be derived from plasmids such as inter alia F, R1, RP1, Col, pBR322, TOL, Ti, etc; cosmids; phages such as lambda, lambdoid, M13, Mu, P1, P22, Qp, T-even, T-odd, T2, T4, T7, etc; plant viruses such as inter alia alfalfa mosaic virus, bromovirus, capillovirus, carlavirus, carnovirus, caulivirus, clostervirus, comovirus, cryptovirus, cucumovirus, dianthovirus, fabavirus, fijivirus, furovirus, geminivirus, hordeivirus, ilarvirus, luteovirus, machlovirus, marafivirus, necrovirus, nepovirus, phytorepvirus, plant rhabdovirus, potexvirus, potyvirus, sobemovirus, tenuivirus, tobamovirus, tobravirus, tomato spotted wilt virus, tombusvirus, tymovirus, etc; or animal viruses such as inter alia adenovirus, arenaviridae, baculoviridae, bimaviridae, bunyaviridae, calciviridae, cardioviruses, coronaviridae, corticoviridae, cystoviridae, Epstein-Barr virus, enteroviruses, filoviridae, flaviviridae, Foot-and-Mouth disease virus, hepadnaviridae, hepatitis viruses, herpesviridae, immunodeficiency viruses, influenza virus, inoviridae, iridoviridae, orthomyxoviridae, papovaviruses, paramyxoviridae, parvoviridae, picomaviridae, poliovirus, polydnaviridae, poxviridae, reoviridae, retroviruses, rhabdoviridae, rhinoviruses, Semliki Forest virus, tetraviridae, togaviridae, toroviridae, vaccinia virus, vescular stomatitis virus, etc. Vectors can be used for cloning and/or for expression of the peptides, parts or analogues thereof of the invention or antibodies of the invention of the invention and might even be used for gene therapy purposes. Vectors comprising one or more nucleic acid molecules according to the invention operably linked to one or more expression-regulating nucleic acid molecules are also covered by the present invention. The choice of vector is dependent on the recombinant procedures followed and the host used. Introduction of vectors in host cells can be effected by inter alia calcium phosphate transfection, virus infection, DEAE-dextran mediated transfection, lipofectamin transfection or electroporation. Vectors may be autonomously replicating or may replicate together with the chromosome into which they have been integrated. Preferably, the vectors contain one or more selection markers. Useful markers are dependent on the host cells of choice and are well known to persons skilled in the art. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the peptides, parts or analogues thereof or antibodies as described above operably linked to one or more nucleic acid molecules encoding proteins or peptides that can be used to isolate these molecules are also covered by the invention. These proteins or peptides include, but are not limited to, glutathione-S-transferase, maltose binding protein, metal-binding polyhistidine, green fluorescent protein, luciferase and beta-galactosidase.

Hosts containing one or more copies of the vectors mentioned above are an additional subject of the present invention. Preferably, the hosts are cells. Preferably, the cells are suitably used for the manipulation and propagation of nucleic acid molecules. Suitable cells include, but are not limited to, cells of mammalian, plant, insect, fungal or bacterial origin. Bacterial cells include, but are not limited to, cells from Gram positive bacteria such as several species of the genera Bacillus, Streptomyces and Staphylococcus or cells of Gram negative bacteria such as several species of the genera Escherichia, such as Escherichia coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells such as Chinese Hamster Ovary (CHO) cells, COS cells, BHK cells or Bowes melanoma cells are preferred in the present invention. Mammalian cells provide expressed proteins with posttranslational modifications that are most similar to natural molecules of mammalian origin. Since the present invention deals with molecules that may have to be administered to humans, a completely human expression system would be particularly preferred. Therefore, even more preferably, the host cells are human cells. Examples of human cells are inter alia HeLa, 911, AT1080, A549, 293 and HEK293T cells. Preferred mammalian cells are human retina cells such as 911 cells or the cell line marketed under the trademark PER.C⁶® (PER.C6 is a registered trademark of Crucell Holland B.V.). For the purposes of this application “PER.C6” refers to cells deposited under number 96022940 or ancestors, passages up-stream or downstream as well as descendants from ancestors of deposited cells, as well as derivatives of any of the foregoing.

In a further aspect, the invention is directed to a peptide, part or analogue thereof according to the invention, or a fusion protein according to the invention or a nucleic acid molecule encoding a peptide, part or analogue thereof according to the invention or a nucleic acid molecule encoding a fusion protein of the invention for use as a medicament. In other words, the invention is directed to a method of detection and/or prevention and/or treatment wherein a peptide, part or analogue thereof according to the invention, or a fusion protein according to the invention or a nucleic acid molecule encoding a peptide, part or analogue thereof according to the invention or a nucleic acid molecule encoding a fusion protein of the invention is used. Preferably, the peptides, parts or analogues thereof of the invention may for example be for use as an immunogen, preferably a vaccine.

If the peptides, parts and analogues thereof of the invention are in the form of a vaccine, they are preferably formulated into compositions. A composition may also comprise more than one peptide of the invention. These peptides may be different or identical and may be linked, covalently or non-covalently, to each other or not linked to each other. They may be linear and/or looped/cyclic. For formulation of such compositions, an immunogenically effective amount of at least one of the peptides of the invention is admixed with a physiologically acceptable carrier suitable for administration to animals including man. The peptides may be covalently attached to each other, to other peptides, to a protein carrier or to other carriers, incorporated into liposomes or other such vesicles, or complexed with an adjuvant or adsorbent as is known in the vaccine art. Alternatively, the peptides are not complexed with the any of the above molecules and are merely admixed with a physiologically acceptable carrier such as normal saline or a buffering compound suitable for administration to animals including man. As with all immunogenic compositions for eliciting antibodies, the immunogenically effective amounts of the peptides of the invention must be determined. Factors to be considered include the immunogenicity of the native peptide, whether or not the peptide will be complexed with or covalently attached to an adjuvant or carrier protein or other carrier and route of administration for the composition, i.e. intravenous, intramuscular, subcutaneous, etc., and number of immunizing doses to be administered. Such factors are known in the vaccine art and it is well within the reach of a skilled artisan to make such determinations without undue experimentation. The peptides, parts or analogues thereof or compositions comprising these compounds may elicit an antibody response upon administrating to human or animal subjects. Such an antibody response protects against further infection by SARS-CoV and/or will retard the onset or progress of the symptoms associated with SARS.

Most preferably, they can be used in the prevention and/or treatment of a condition resulting from a SARS-CoV.

In yet another aspect, antibodies of the invention can be used as a medicament, preferably in the treatment of a condition resulting from a SARS-CoV. In a specific embodiment, they can be used with any other medicament available to treat a condition resulting from a SARS-CoV. In other words, the invention also pertains to a method of prevention and/or treatment, wherein the antibodies, fragments or functional variants thereof according to the invention are used.

The antibodies of the invention can also be used for detection of the SARS-CoV, e.g. for diagnostic purposes. Therefore, the invention provides a diagnostic test method for determining the presence of SARS-CoV in a sample, characterized in that said sample is put into contact with an antibody according to the invention. Preferably the antibody is contacted with the sample under conditions which allow the formation of an immunological complex between the antibodies and SARS-CoV or fragments or (poly)peptides thereof that may be present in the sample. The formation of an immunological complex, if any, indicating the presence of SARS-CoV in the sample, is then detected and measured by suitable means. The sample may be a biological sample including, but not limited to blood, serum, urine, tissue or other biological material from (potentially) infected subjects, or a nonbiological sample such as water, drink, etc. The (potentially) infected subjects may be human subjects, but also animals that are suspected as carriers of SARS-CoV might be tested for the presence of SARS-CoV using these antibodies. Detection of binding may be according to standard techniques known to a person skilled in the art, such as an ELISA, Western blot, RIA, etc. The antibodies may suitably be included in kits for diagnostic purposes. It is therefore another aspect of the invention to provide a kit of parts for the detection of SARS-CoV comprising an antibody according to the invention.

The antibodies of the invention may be used to purify SARS-CoV or a fragment thereof. Antibodies against peptides of specific proteins of SARS-CoV such as the proteins mentioned herein, may also be used to purify the proteins. Purification techniques for viruses and proteins are well known to the skilled artisan.

Also the peptides of the invention can be used directly for the detection of SARS-CoV recognizing antibodies, for instance for diagnostic purposes. It is therefore an object of the invention to provide methods for determining the presence of antibodies recognizing SARS-CoV in a sample, characterized in that said sample is put into contact with a peptide (or part thereof, analogue thereof, fusion protein or conjugate) of the invention. Preferably the peptide is contacted with the sample under conditions which allow the formation of an immunological complex between the peptide and any antibodies to SARS-CoV that may be present in the sample. The formation of an immunological complex, if any, indicating the presence of antibodies to SARS-CoV in the sample, is then detected and measured by suitable means. Such methods include, inter alia, homogeneous and heterogeneous binding immunoassays, such as radioimmunoassays (RIA), ELISA and Western blot analyses. Further, the assay protocols using the novel peptides allow for competitive and non-competitive binding studies to be performed. The sample used in the diagnostic test method may for instance be blood, urine, tissue material or other material from (potentially) infected subjects. The peptide may however also be used to diagnose prior exposure to the SARS-CoV. Preferred assay techniques, especially for large-scale clinical screening of patient sera and blood and blood-derived products are ELISA and Western blot techniques. ELISA tests are particularly preferred. For use as reagents in these assays, the peptides of the invention are conveniently bonded to the inside surface of microtiter wells. The peptides may be directly bonded to the microtiter well. However, maximum binding of the peptides to the wells might be accomplished by pretreating the wells with polylysine prior to the addition of the peptides. Furthermore, the novel peptides may be covalently attached by known means to a carrier protein, such as BSA, with the resulting conjugate being used to coat the wells. Generally the peptides are used in a concentration of between 0.01 to 100 μg/ml for coating, although higher as well as lower amounts may also be used. Samples are then added to the peptide coated wells where an immunological complex forms if antibodies to SARS-CoV are present in the sample. A signal generating means may be added to aid detection of complex formation. A detectable signal is produced if SARS-CoV specific antibodies are present in the sample.

### EXAMPLES

### Example 1

### Identification of Epitopes Recognized by Human Sera of Individuals Which have been and/or are Infected by SARS-CoV by Means of PEPSCAN-ELISA

Overlapping 15-mer linear and looped/cyclic peptides were synthesized from several proteins of SARS-CoV Urbani. The complete genome of SARS-CoV Urbani can be found under EMBL-database accession number AY278741, “SARS coronavirus Urbani, complete genome.” The coding sequence (CDS) of the proteins is also shown under EMBL-database accession number AY278741.

Linear as well as looped/cyclic peptides were prepared from the SARS-CoV Urbani proteins called protein X1 (the protein-id of protein X1 is AAP13446, see also SEQ ID NO:1), protein X2 (the protein-id of protein X2 is AAP13447, see also SEQ ID NO:2), E protein (the protein-id of the envelope protein, E protein, is AAP13443, see also SEQ ID NO:3), M protein (the protein-id of the small membrane protein, M protein, is AAP13444, see also SEQ ID NO:4), protein X3 (the protein-id of protein X3 is AAP13448, see also SEQ ID NO:5), protein X4 (the protein-id of protein X4 is AAP13449, see also SEQ ID NO:6), protein X5 (the protein-id of protein X5 is AAP13450, see also SEQ ID NO:7), and N protein (the protein-id of the nucleocapsid protein, N protein, is AAP13445, see also SEQ ID NO:8).

Next, the prepared peptides were screened using credit-card format mini-PEPSCAN cards (455 peptide formats/card) as described previously (Slootstra et al., 1996; WO 93/09872). All peptides were acetylated at the amino terminus.

In all looped peptides position-2 and position-14 were replaced by a cysteine (acetyl-XCXXXXXXXXXXCX-minicard). If other cysteines besides the cysteines at position-2 and position-14 were present in a prepared peptide, the other cysteines were replaced by an alanine. The looped peptides were synthesized using standard Fmoc-chemistry and deprotected using trifluoric acid with scavengers. Subsequently, the deprotected peptides were reacted on the cards with an 0.5 mM solution of 1,3-bis(bromomethyl)benzene in ammonium bicarbonate (20 mM, pH 7.9)/acetonitril (1:1 (v/v)). The cards were gently shaken in the solution for 30-60 minutes, while completely covered in the solution. Finally, the cards were washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

The binding of antibodies to each linear and looped peptide was tested in a PEPSCAN-based enzyme-linked immuno assay (ELISA). The 455-well creditcard-format polypropylene cards, containing the covalently linked peptides, were incubated with serum (diluted 1/1000 in blocking solution which contains 5% horse-serum (v/v) and 5% ovalbumin (w/v)) (4° C., overnight). Before use, the serum was heat-inactivated at 56° C. for 1 hour. After washing the peptides were incubated with anti-human antibody peroxidase (dilution 1/1000) (1 hour, 25° C.), and subsequently, after washing the peroxidase substrate 2,2′-azino-di-3-ethylbenzthiazoline sulfonate (ABTS) and 2 μl/ml 3% H₂O₂ were added. After 1 hour the color development was measured. The color development of the ELISA was quantified with a CCD-camera and an image processing system. The setup consists of a CCD-camera and a 55 mm lens (Sony CCD Video Camera XC-77RR, Nikon micro-nikkor 55 mm f/2.8 lens), a camera adaptor (Sony Camera adaptor DC-77RR) and the Image Processing Software package Optimas , version 6.5 (Media Cybernetics, Silver Spring, Md. 20910, U.S.A.). Optimas runs on a pentium II computer system.

The serum derived from an individual that has been infected by SARS-CoV and has recovered from SARS (serum called SARS-green) and the serum derived from an individual in which the virus was still detectable by PCR and who suffered a prolonged and severe form of the illness (serum called SARS-yellow) and the sera derived from individuals which have been and/or are still infected by SARS-CoV (the sera called 1a (individual 1, early serum), 1b (individual 1, late serum), 2 (individual 2), 6 (individual 6), 37 (individual 37), 62 (individual 62) and London) were tested for binding to the 15-mer linear and looped/cyclic peptides synthesized as described supra. Additionally, two control sera were tested for binding the 15-mer linear and looped/cyclic peptides synthesized as described supra. One control serum was a pooled serum of ten healthy LUMC (Leids Universitair Medisch Centrum) hospital workers and the second control serum was a commercial negative donor pooled serum from the Dutch bloodbank. Next to that, a rabbit serum obtained by immunizing a rabbit with the SARS-CoV strain Frankfurt 1 was tested for binding the 15-mer linear and looped/cyclic peptides synthesized as described supra. The SARS-CoV was concentrated and partially purified by sucrose-gradient ultracentrifugation. After that, the purified SARS-CoV was gamma-irradiated for inactivation (approximately 35 kGy), mixed with complete Freund adjuvans and used for immunization purposes. Immunization was performed according to the art well known to the skilled artisan.

See Table 1 for results of the binding of the different above sera to linear peptides of protein X1 of SARS-CoV Urbani. See Table 2 for results of the binding of the different above sera to looped/cyclic peptides of protein X1 of SARS-CoV Urbani.

See Table 3 for results of the binding of the different above sera to linear peptides of protein X2 of SARS-CoV Urbani. See Table 4 for results of the binding of the different above sera to looped/cyclic peptides of protein X2 of SARS-CoV Urbani.

See Table 5 for results of the binding of the different above sera to linear peptides of protein E of SARS-CoV Urbani. See Table 6 for results of the binding of the different above sera to looped/cyclic peptides of protein E of SARS-CoV Urbani.

See Table 7 for results of the binding of the different above sera to linear peptides of protein M of SARS-CoV Urbani. See Table 8 for results of the binding of the different above sera to looped/cyclic peptides of protein M of SARS-CoV Urbani.

See Table 9 for results of the binding of the different above sera to linear peptides of protein X3 of SARS-CoV Urbani. See Table 10 for results of the binding of the different above sera to looped/cyclic peptides of protein X3 of SARS-CoV Urbani.

See Table 11 for results of the binding of the different above sera to linear peptides of protein X4 of SARS-CoV Urbani. See Table 12 for results of the binding of the different above sera to looped/cyclic peptides of protein X4 of SARS-CoV Urbani.

See Table 13 for results of the binding of the different above sera to linear peptides of protein X5 of SARS-CoV Urbani. See Table 14 for results of the binding of the different above sera to looped/cyclic peptides of protein X5 of SARS-CoV Urbani.

See Table 15 for results of the binding of the different above sera to linear peptides of protein N of SARS-CoV Urbani.

See Table 16 for results of the binding of the different above sera to looped/cyclic peptides of protein N of SARS-CoV Urbani.

See Table 17 for results of the binding of the two control sera to linear and looped/cyclic peptides of protein X1 of SARS-CoV Urbani. The following peptides were recognized by at least one of the control sera in linear form, looped/cyclic form or in both forms:

| | |
|---|---|
| DNASPASTVHATATI, | (SEQ ID NO: 421) |
| NASPASTVHATATIP, | (SEQ ID NO: 422) |
| ASPASTVHATATIPL, | (SEQ ID NO: 423) |
| SPASTVHATATIPLQ, | (SEQ ID NO: 424) |
| PASTVHATATIPLQA, | (SEQ ID NO: 425) |
| ASTVHATATIPLQAS, | (SEQ ID NO: 426) |
| STVHATATIPLQASL, | (SEQ ID NO: 427) |
| TVHATATIPLQASLP, | (SEQ ID NO: 428) |
| VHATATIPLQASLPF, | (SEQ ID NO: 429) |
| AVFQSATKIIALNKR, | (SEQ ID NO: 430) |
| VFQSATKIIALNKRW, | (SEQ ID NO: 431) |
| FQSATKIIALNKRWQ, | (SEQ ID NO: 432) |
| QSATKIIALNKRWQL, | (SEQ ID NO: 433) |
| SATKIIALNKRWQLA, | (SEQ ID NO: 434) |
| ATKIIALNKRWQLAL, | (SEQ ID NO: 435) |
| TKIIALNKRWQLALY, | (SEQ ID NO: 436) |
| KIIALNKRWQLALYK, | (SEQ ID NO: 437) |
| IIALNKRWQLALYKG | (SEQ ID NO: 438) |
| and | |
| IALNKRWQLALYKGF. | (SEQ ID NO: 439) |

See Table 18 for results of the binding of the two control sera to linear and looped/cyclic peptides of protein X2 of SARS-CoV Urbani. The following peptides were recognized by at least one of the control sera in linear form, looped/cyclic form or in both forms:

| | |
|---|---|
| MMPTTLFAGTHITMT, | (SEQ ID NO: 440) |
| MPTTLFAGTHITMTT, | (SEQ ID NO: 441) |
| PTTLFAGTHITMTTV, | (SEQ ID NO: 442) |
| TTLFAGTHITMTTVY, | (SEQ ID NO: 443) |
| TLFAGTHITMTTVYH, | (SEQ ID NO: 444) |
| LFAGTHITMTTVYHI, | (SEQ ID NO: 445) |
| FAGTHITMTTVYHIT, | (SEQ ID NO: 446) |
| AGTHITMTTVYHITV | (SEQ ID NO: 447) |
| and | |
| GTHITMTTVYHITVS. | (SEQ ID NO: 448) |

See Table 19 for results of the binding of the two control sera to linear and looped/cyclic peptides of protein E of SARS-CoV Urbani.

See Table 20 for results of the binding of the two control sera to linear and looped/cyclic peptides of protein M of SARS-CoV Urbani. The following peptides were recognized by at least one of the control sera in linear form, looped/cyclic form or in both forms:

| | |
|---|---|
| GTITVEELKQLLEQW, | (SEQ ID NO: 449) |
| TITVEELKQLLEQWN, | (SEQ ID NO: 450) |
| ITVEELKQLLEQWNL, | (SEQ ID NO: 451) |
| TVEELKQLLEQWNLV, | (SEQ ID NO: 452) |
| VEELKQLLEQWNLVI, | (SEQ ID NO: 453) |
| EELKQLLEQWNLVIG, | (SEQ ID NO: 454) |
| VIGAVIIRGHLRMAG, | (SEQ ID NO: 455) |
| IGAVIIRGHLRMAGH, | (SEQ ID NO: 456) |
| GAVIIRGHLRMAGHP, | (SEQ ID NO: 457) |
| AVIIRGHLRMAGHPL, | (SEQ ID NO: 458) |
| VIIRGHLRMAGHPLG, | (SEQ ID NO: 459) |
| IIRGHLRMAGHPLGR, | (SEQ ID NO: 460) |
| IRGHLRMAGHPLGRC, | (SEQ ID NO: 461) |
| RGHLRMAGHPLGRCD, | (SEQ ID NO: 462) |
| GHLRMAGHPLGRCDI | (SEQ ID NO: 463) |
| and | |
| HLRMAGHPLGRCDIK. | (SEQ ID NO: 464) |

See Table 21 for results of the binding of the two control sera to linear and looped/cyclic peptides of protein X3 of SARS-CoV Urbani.

See Table 22 for results of the binding of the two control sera to linear and looped/cyclic peptides of protein X4 of SARS-CoV Urbani. The following peptides were recognized by at least one of the control sera in linear form, looped/cyclic form or in both forms:

| | |
|---|---|
| TYEGNSPFHPLADNK, | (SEQ ID NO: 465) |
| YEGNSPFHPLADNKF, | (SEQ ID NO: 466) |
| EGNSPFHPLADNKFA, | (SEQ ID NO: 467) |
| GNSPFHPLADNKFAL, | (SEQ ID NO: 468) |
| NSPFHPLADNKFALT | (SEQ ID NO: 469) |
| and | |
| SPFHPLADNKFALTC. | (SEQ ID NO: 470) |

See Table 23 for results of the binding of the two control sera to linear and looped/cyclic peptides of protein X5 of SARS-CoV Urbani. The following peptides were recognized by at least one of the control sera in linear form, looped/cyclic form or in both forms:

| | |
|---|---|
| IARCWYLHEGHQTAA, | (SEQ ID NO: 471) |
| ARCWYLHEGHQTAAF, | (SEQ ID NO: 472) |
| RCWYLHEGHQTAAFR, | (SEQ ID NO: 473) |
| CWYLHEGHQTAAFRD, | (SEQ ID NO: 474) |
| WYLHEGHQTAAFRDV, | (SEQ ID NO: 475) |
| YLHEGHQTAAFRDVL, | (SEQ ID NO: 476) |
| LHEGHQTAAFRDVLV | (SEQ ID NO: 477) |
| and | |
| HEGHQTAAFRDVLVV. | (SEQ ID NO: 478) |

See Table 24 for results of the binding of the two control sera to linear and looped/cyclic peptides of protein N of SARS-CoV Urbani. The following peptides were recognized by at least one of the control sera in linear form, looped/cyclic form or in both forms:

| | |
|---|---|
| AATVLQLPQGTTLPK, | (SEQ ID NO: 479) |
| ATVLQLPQGTTLPKG, | (SEQ ID NO: 480) |
| TVLQLPQGTTLPKGF, | (SEQ ID NO: 481) |
| NSTPGSSRGNSPARM, | (SEQ ID NO: 482) |
| STPGSSRGNSPARMA, | (SEQ ID NO: 483) |
| TPGSSRGNSPARMAS, | (SEQ ID NO: 484) |
| PGSSRGNSPARMASG, | (SEQ ID NO: 485) |
| GSSRGNSPARMASGG, | (SEQ ID NO: 486) |
| LDDKDPQFKDNVILL, | (SEQ ID NO: 487) |
| DDKDPQFKDNVILLN, | (SEQ ID NO: 488) |
| DKDPQFKDNVILLNK, | (SEQ ID NO: 489) |
| KDPQFKDNVILLNKH | (SEQ ID NO: 490) |
| and | |
| DPQFKDNVILLNKHI. | (SEQ ID NO: 491) |

In Table 25 the results of the binding of the rabbit serum to linear and looped/cyclic peptides of protein X1 of SARS-CoV Urbani are shown. The following peptides were recognized by the rabbit serum in linear form, looped/cyclic form or in both forms: AVFQSATKIIALNKR (SEQ ID NO:492), VFQSATKIIALNKRW (SEQ ID NO:493), FQSATKIIALNKRWQ (SEQ ID NO:494), QSATKIIALNKRWQL (SEQ ID NO:495), SATKIIALNKRWQLA (SEQ ID NO:496), ATKIIALNKRWQLAL (SEQ ID NO:497), TKIIALNKRWQLALY (SEQ ID NO:498), KIIALNKRWQLALYK (SEQ ID NO:499), IIALNKRWQLALYKG (SEQ ID NO:500), IALNKRWQLALYKGF (SEQ ID NO:501), ALNKRWQLALYKGFQ (SEQ ID NO:502), LNKRWQLALYKGFQF (SEQ ID NO:503), NKRWQLALYKGFQFI (SEQ ID NO:504), LQCINACRIIMRCWL (SEQ ID NO:505), QCINACRIIMRCWLC (SEQ ID NO:506), CINACRIIMRCWLCW (SEQ ID NO:507), INACRIIMRCWLCWK (SEQ ID NO:508), NACRIIMRCWLCWKC (SEQ ID NO:509) and ACRIIMRCWLCWKCK (SEQ ID NO:510).

In Table 26 the results of the binding of the rabbit serum to linear and looped/cyclic peptides of protein X2 of SARS-CoV Urbani are shown. The following peptides were recognized by the rabbit serum in linear form, looped/cyclic form or in both forms: TAFQHQNSKKTTKLV (SEQ ID NO:511), AFQHQNSKKTTKLVV (SEQ ID NO:512), FQHQNSKKTTKLVVI (SEQ ID NO:513), QHQNSKKTTKLVVIL (SEQ ID NO:514), HQNSKKTTKLVVILR (SEQ ID NO:515), QNSKKTTKLVVILRI (SEQ ID NO:516), NSKKTTKLVVILRIG (SEQ ID NO:517), SKKTTKLVVILRIGT (SEQ ID NO:518), KKTTKLVVILRIGTQ (SEQ ID NO:519), KTTKLVVILRIGTQV (SEQ ID NO:520) and TTKLVVILRIGTQVL (SEQ ID NO:521).

In Table 27 the results of the binding of the rabbit serum to linear and looped/cyclic peptides of protein E of SARS-CoV Urbani are shown.

In Table 28 the results of the binding of the rabbit serum to linear and looped/cyclic peptides of protein M of SARS-CoV Urbani are shown. The following peptides were recognized by the rabbit serum in linear form, looped/cyclic form or in both forms: MADNGTITVEELKQL (SEQ ID NO:522), ADNGTITVEELKQLL (SEQ ID NO:523), DNGTITVEELKQLLE (SEQ ID NO:524), NGTITVEELKQLLEQ (SEQ ID NO:525), GTITVEELKQLLEQW (SEQ ID NO:526), TITVEELKQLLEQWN (SEQ ID NO:527), ITVEELKQLLEQWNL (SEQ ID NO:528), TVEELKQLLEQWNLV (SEQ ID NO:529) and VEELKQLLEQWNLVI (SEQ ID NO:530).

In Table 29 the results of the binding of the rabbit serum to linear and looped/cyclic peptides of protein X3 of SARS-CoV Urbani are shown.

In Table 30 the results of the binding of the rabbit serum to linear and looped/cyclic peptides of protein X4 of SARS-CoV Urbani are shown. The following peptides were recognized by the rabbit serum in linear form, looped/cyclic form or in both forms: FACADGTRHTYQLRA (SEQ ID NO:531), ACADGTRHTYQLRAR (SEQ ID NO:532), CADGTRHTYQLRARS (SEQ ID NO:533), ADGTRHTYQLRARSV (SEQ ID NO:534), DGTRHTYQLRARSVS (SEQ ID NO:535), GTRHTYQLRARSVSP (SEQ ID NO:536), TRHTYQLRARSVSPK (SEQ ID NO:537), RHTYQLRARSVSPKL (SEQ ID NO:538), HTYQLRARSVSPKLF (SEQ ID NO:539), TYQLRARSVSPKLFI (SEQ ID NO:540), YQLRARSVSPKLFIR (SEQ ID NO:541), QLRARSVSPKLFIRQ (SEQ ID NO:542), LRARSVSPKLFIRQE (SEQ ID NO:543) and RARSVSPKLFIRQEE (SEQ ID NO:544).

In Table 31 the results of the binding of the rabbit serum to linear and looped/cyclic peptides of protein X5 of SARS-CoV Urbani are shown.

In Table 32 the results of the binding of the rabbit serum to linear and looped/cyclic peptides of protein N of SARS-CoV Urbani are shown. The following peptides were recognized by the rabbit serum in linear form, looped/cyclic form or in both forms: NGPQSNQRSAPRITF (SEQ ID NO:592), GPQSNQRSAPRITFG (SEQ ID NO:593), PQSNQRSAPRITFGG (SEQ ID NO:594), QSNQRSAPRITFGGP (SEQ ID NO:595), SGPDDQIGYYRRATR (SEQ ID NO:545), GPDDQIGYYRRATRR (SEQ ID NO:546), PDDQIGYYRRATRRV (SEQ ID NO:547), DDQIGYYRRATRRVR (SEQ ID NO:548), DQIGYYRRATRRVRG (SEQ ID NO:549), QIGYYRRATRRVRGG (SEQ ID NO:550), IGYYRRATRRVRGGD (SEQ ID NO:551), GYYRRATRRVRGGDG (SEQ ID NO:552), RNSTPGSSRGNSPAR (SEQ ID NO:553), NSTPGSSRGNSPARM (SEQ ID NO:554), STPGSSRGNSPARMA (SEQ ID NO:555), TPGSSRGNSPARMAS (SEQ ID NO:556), PGSSRGNSPARMASG (SEQ ID NO:557), GSSRGNSPARMASGG (SEQ ID NO:558), PRQKRTATKQYNVTQ (SEQ ID NO:559), RQKRTATKQYNVTQA (SEQ ID NO:560), QKRTATKQYNVTQAF (SEQ ID NO:561), KRTATKQYNVTQAFG (SEQ ID NO:562), RTATKQYNVTQAFGR (SEQ ID NO:563), TATKQYNVTQAFGRR (SEQ ID NO:564), ATKQYNVTQAFGRRG (SEQ ID NO:565), TKQYNVTQAFGRRGP (SEQ ID NO:566), KQYNVTQAFGRRGPE (SEQ ID NO:567), QYNVTQAFGRRGPEQ (SEQ ID NO:568), YNVTQAFGRRGPEQT (SEQ ID NO:569), NVTQAFGRRGPEQTQ (SEQ ID NO:570), VTQAFGRRGPEQTQG (SEQ ID NO:571) and TQAFGRRGPEQTQGN (SEQ ID NO:572).

The oligopeptides identified by the rabbit serum might be (additional) good candidates to represent epitopes of the SARS-CoV. The peptides may be advantageously used in diagnostic test methods as described herein. They may also be used in therapy and/or prevention of conditions resulting from an infection with SARS-CoV as described herein.

Relevant binding of a peptide to a serum was calculated as follows. The average OD-value for each serum was calculated for each protein (sum of OD-values of all peptides/total number of peptides). Next, the standard deviation of this average was calculated. The standard deviation was multiplied by 2 and the obtained value was added to the average value to obtain the correction factor. The OD-value of each peptide was then divided by this correction factor. If a value of 0.9 or higher was found, then relevant binding was considered to be present between the specific peptide and the respective serum. Particularly, domains (response of clustering of reactive peptides reactive with several individual sera) comprising several relevant peptides were claimed in the present invention. These domains are indicated (colored grey) in the above-mentioned tables.

Any of the above peptides could form the basis for diagnostic kits comprising the peptides, vaccines (as peptide, DNA, or vector vaccine) or for raising neutralizing antibodies to treat and/or prevent SARS or for raising antibodies to detect SARS-CoV.

### Example 2

### Selection of Phage Carrying Single-Chain Fv Fragments Specifically Recognizing SARS-CoV

Antibody fragments were selected using antibody phage display libraries and technology, essentially as described in U.S. Pat. No. 6,265,150 and in WO 98/15833, both of which are incorporated herein in their entirety. All procedures were performed at room temperature unless stated otherwise. An inactivated SARS-CoV preparation (Frankfurt 1 strain) was prepared as follows. Medium from Vero cells which were infected with SARS-CoV strain Frankfurt 1 was harvested as soon as cyotopathic effect (CPE) was observed. Cell debris was removed by centrifugation of the harvested medium for 15 minutes at 3000 rpm. The obtained supernatant was collected, spun again for 15 minutes at 3000 rpm and transferred to a clean tube. Subsequently, ultracentrifuge tubes were filled with 10 ml sterile 25% glycerol in PBS. 20 ml of the cleared supernatant was gently applied on the glycerol cushion and the tubes were spun for 2 hours at 20,000 rpm at 4° C. The supernatant was discarded and the virus pellets were resuspended in 1 ml TNE buffer (10 mM Tris-HCl pH 7.4, 1 mM EDTA, 200 mM NaCl) and stored at −80° C. Next, the resuspended virus pellets were gamma-irradiated at 45 kGy on dry ice. Subsequently, they were tested for the absence of infectivity in cell culture. If absence of infectivity was established, the thus obtained inactivated SARS-CoV preparation was used for selection of single-chain phage antibodies specifically binding to SARS-CoV.

The inactivated virus preparation and heat-inactivated fetal bovine serum (FBS) were coated overnight at 4° C. onto the surface of separate Maxisorp™ plastic tubes (Nunc). The tubes were blocked for two hours in 3 ml PBS containing 2% FBS and 2% fat free milk powder (2% PBS-FM). After two hours the FBS-coated tube was emptied and washed three times with PBS. To this tube, 500 μl (approximately 10¹³ cfu) of a phage display library expressing single-chain Fv fragments (scFvs) essentially prepared as described by De Kruif et al. (1995a) and references therein (which are incorporated herein in their entirety), 500 μl 4% PBS-FM and 2 ml 2% PBS-FM were added. The tube was sealed and rotated slowly at room temperature for two hours. Subsequently, the obtained blocked phage library (3 ml) was transferred to a SARS-CoV preparation-coated tube that had been washed three times with PBS. Tween-20 was added to a final concentration of 0.05% and binding was allowed to proceed for two hours on a slowly rotating wheel at room temperature or at 37° C. The tube was emptied and washed ten times with PBS containing 0.05% Tween-20, followed by washing ten times with PBS. 1 ml glycine-HCL (0.05 M, pH 2.2) was added to elute bound phages, and the tube was rotated slowly for ten minutes. For neutralization purposes, the eluted phages were added to 500 μl 1 M Tris-HCl pH 7.4. To this mixture, 5 ml of exponentially growing XL-1 blue bacterial culture was added. The obtained culture was incubated for thirty minutes at 37° C. without shaking. Then, the bacteria were plated on TYE agar plates containing ampicillin, tetracycline and glucose. After overnight incubation of the plates at 37° C., the colonies were scraped from the plates and used to prepare an enriched phage library, essentially as described by De Kruif et al. (1995a) and WO 02/103012 (both are incorporated by reference herein). Briefly, scraped bacteria were used to inoculate 2TY medium containing ampicillin, tetracycline and glucose and grown at a temperature of 37° C. to an OD600 nm of ˜0.3. CT or VCSM13 helper phages were added and allowed to infect the bacteria after which the medium was changed to 2TY containing ampicillin, tetracycline and kanamycin. Incubation was continued overnight at 30° C. The next day, the bacteria were removed from the 2TY medium by centrifugation after which the phages in the obtained supernatant were precipitated using polyethylene glycol 6000/NaCl. Finally, the phages were dissolved in a small volume of PBS containing 1% BSA, filter-sterilized and used for a next round of selection. The selection/re-infection procedure was performed two or three times. After each round of selection, individual E. coli colonies were used to prepare monoclonal phage antibodies. Essentially, individual colonies were grown to log-phase and infected with VCSM13 helper phages after which phage antibody production was allowed to proceed overnight. Phage antibody containing supernatants were tested in ELISA for binding activity to the SARS-CoV preparation which was coated to 96-well plates. In the above described selection, the phage antibodies called SC03-001, SC03-002, SC03-003, SC03-005, SC03-006, SC03-007, SC03-008, SC03-009, SC03-0010, SC03-012, SC03-013, SC03-014 and SC03-015 were obtained.

To overcome selection of previously identified phage antibodies, alternative selections in the presence of scFvs corresponding to the previous selected phage antibodies were performed as follows. ScFvs of the phage antibodies SC03-001, SC03-002, SC03-003, SC03-005, SC03-006, SC03-007, SC03-008, SC03-009, SC03-010, SC03-012, SC03-013, SC03-014 and SC03-015 were produced as described before in De Kruif et al. (1995b). The amino acid sequence of the scFvs is shown in SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584 and SEQ ID NO:585, respectively. The buffer of the scFvs was adjusted to 1×PBS. Then the scFvs were mixed with 500 μl (approximately 10¹³ cfu) of a phage display library expressing single-chain Fv fragments essentially prepared as described by De Kruif et al. (1995a) and references therein (which are incorporated herein in their entirety). Next, the obtained mixture was blocked in an FBS-coated tube as described above and subsequently selection was carried out with the obtained blocked mixture essentially as described above for the blocked phage library. In this alternative selection, the phage antibodies called SC03-016, SC03-017 and SC03-018 were obtained.

```
SC03-001 (SEQ ID NO: 573):
                                                        SMAEVQLVESGGGLVKPGGSLRLSCAASGFTF
SGYSMNWVRQAPGKGLEWVSSISGGSTYYADSRKGRFTISRDNSKNTLYLQMNNLRAEDT
AVYYCARHRFRHVFDYWGQGTLVTVLEGTGGSGGTGSGTGTSELTQSPSSLSASVGDRVTI
TCRASQSISSYLNWYQQKPGKAPKLLIYAASSLQSGVPSRFSGSGSGTDFTLTISSLQPEDFAT
YYCQQSYSTPPTFGQGTKVEIKRAAA
SC03-002 (SEQ ID NO: 574):
                                                        SMAEVQLVESGGGLVKPGGSLRLSCAASGFTF
SGYSMSWVRQAPGKGLEWVGRTRNKANSYTTEYAASVKGRFTISRDDSKNSLYLQMNSLK
TEDTAVYYCARYYSRSLKAFDYWGQGTLVTVLEGTGGSGGTGSGTGTSELTQSPSSLSASV
GDRVTITCRASQSISSYLNWYQQKPGKAPKLLIYAASSLQSGVPSRFSGSGSGTDFTLTISSLQ
PEDFATYYCQQSYSTPPTFGQGTKVEIKRAAA
SC03-003 (SEQ ID NO: 575):
                                                        SMAEVQLVESGGGLVQPGGSLRLSCAASGFTF
SSYPMNWVRQAPGKGLEWVSAISGSGGSTYYADSVKGRFTISRDNSKNTLYLQMNSLRAE
DTAVYYCARRSYFRRFDYWGQGTLVTVLEGTGGSGGTGSGTGTSELTQSPSSLSASVGDRV
TITCRASQSISSYLNWYQQKPGKAPKLLIYAASSLQSGVPSRFSGSGSGTDFTLTISSLQPEDF
ATYYCQQSYSTPPTFGQGTKVEIKRAAA
SC03-005 (SEQ ID NO: 576):
                                                          SMAEVQLVESGGGLIQPGGSLRLSCAASGFT
FSGYTMSWVRQAPGQGLEWVSSISGGSTYYADSRKGRFTISRDNSKNTLYLQMNNLRAED
TAVYYCAKGGGRPYNPFDYWGQGTLVTVLEGTGGSGGTGSGTGTSELTQSPSSLSASVGD
RVTITCRASQSISSYLNWYQQKPGKAPKLLIYAASSLQSGVPSRFSGSGSGTDFTLTISSLQPE
DFATYYCQQSYSTPPTFGQGTKVEIKRAAA
SC03-006 (SEQ ID NO: 577):
                                                          SMAEVQLVESGGGLVQPGGSLRLSCAASGFT
FSGYPMHWVRQAPGKGLEWVAVISYDGSNKYYADSVKGRFTISRDNSKNTLYLQMNSLRA
EDTAVYYCAKDGSPRTPSFDYWGQGTLVTVLEGTGGSGGTGSGTGTSELDIQMTQSPHSLS
ASVGDRVTITCRASQGISNYLAWYQQKPGKVPKLLIYAASTLQSGVPSRFSGSGSGTDFTLTI
SSLQPEDVGVYYCQQRFRTPVTFGQGTKLEIKRAAA
SC03-007 (SEQ ID NO: 578):
                                                        SMAEVQLVESGGGLVQPRGSLRLSCAASGFTF
SDYRMNWVRQAPGKGLERVAVISYDGSNKYYADSVKGRFTISRDNSKNTLYLQMNSLRAE
DTAVYYCARGYWTSLTGFDYWGQGTLVTVLEGTGGSGGTGSGTGTSELTQSPSSLSASVG
DRVTITCRASQSISSYLNWYQQKPGKAPKLLIYAASSLQSGVPSRFSGSGSGTDFTLTISSLQP
EDFATYYCQQSYSTPPTFGQGTKVEIKRAAA
SC03-008 (SEQ ID NO: 579):
                                                        SMAEVQLVESGGGVVQPGRSLRLSCAASGFTF
SSYPMNWVRQAPGKGLEWVSAISGSGGSTYYADSVKGRFTISRDNSKNTLYLQMNSLRAE
DTAVYYCARRVRPRRFDYWGQGTLVTVLEGTGGSGGTGSGTGTSELTQSPSSLSASVGDR
VTITCRASQSISSYLNWYQQKPGKAPKLLIYAASSLQSGVPSRFSGSGSGTDFTLTISSLQPED
FATYYCQQSYSTPPTFGQGTKVEIKRAAA
SC03-009 (SEQ ID NO: 580):
                                                        SMAEVQLVESGGGVVQPGRSLRLSCAASGFTF
SDYPMNWVRQAPGKGLEWVSSISGSGGSTYYADSVKGRFTISRDNSKNTLYLQMNSLRAE
DTAVYYCAKGLFMVTTYAFDYWGQGTLVTVLEGTGGSGGTGSGTGTSELTQSPSSLSASV
GDRVTITCRASQSISSYLNWYQQKPGKAPKLLIYAASSLQSGVPSRFSGSGSGTDFTLTISSLQ
PEDFATYYCQQSYSTPPTFGQGTKVEIKRAAA
SC03-010 (SEQ ID NO: 581):
                                                        SMAEVQLVESGGGVVQPGRSLRLSCATSGFTF
SGYTMHWVRQAPGKGLEWVSSISGGSTYYADSRKGRFTISRDNSKNTLYLQMNNLRAEDT
AVYYCAKGGGLPYLSFDYWGQGTLVTVLEGTGGSGGTGSGTGTSELTQSPSSLSASVGDR
VTITCRASQSISSYLNWYQQKPGKAPKLLIYAASSLQSGVPSRFSGSGSGTDFTLTISSLQPED
FATYYCQQSYSTPPTFGQGTKVEIKRAAA
SC03-012 (SEQ ID NO: 582):
                                                            AMAQVQLVQSGAEVKKPGASVKVSCKASGY
TFTSYGISWVRQAPGQGLEWMGWISAYNGNTNYAQKLQGRVTMTTDTSTSTAYMELSSLR
SDDTAVYYCARMFRKSSFDSWGQGTLVTVSRGGGGSGGGGSGGGGSSELTQDPAVSVALG
QTVRITCQGDSLRSYYASWYQQKPGQAPVLVIYGKNNRPSGIPDRFSGSSSGNTASLTITGA
QAEDEADYYCNSRDSSGNHVVFGGGTKLTVLGAAA
SC03-013 (SEQ ID NO: 583):
                                                      AMAEVQLVESGGGLVQPGGSLRLSCAASGFTF
SDHYMDWVRQAPGKGLEWVGRTRNKANSYTTEYAASVKGRFTISRDDSKNSLYLQMNSL
KTEDTAVYYCAKGLTPLYFDYWGQGTLVTVSSGGGGSGGGGSGGGGSEIELTQSPSSLSAS
VGDRVTITCRASQSISSYLNWYQQKPGKAPKLLIYAASSLQSGVPSRFSGSGSGTDFTLTISSL
QPEDFATYYCQQSYSTPPTFGQGTKVEIKRAAA
SC03-014 (SEQ ID NO: 584):
                                                      AMAEVQLVESGGGLVQPGGSLRLSCAASGFTF
SDHYMDWVRQAPGKGLEWVGRTRNKANSYTTEYAASVKGRFTISRDDSKNSLYLQMNSL
KTEDTAVYYCARGISPFYFDYWGQGTLVTVSSGGGGSGGGGSGGGGSEIELTQSPSSLSASV
GDRVTITCRASQSISSYLNWYQQKPGKAPKLLIYAASSLQSGVPSRFSGSGSGTDFTLTISSLQ
PEDFATYYCQQSYSTPPTFGQGTKVEIKRAAA
SC03-015 (SEQ ID NO: 585):
                                                      AMAEVQLVESGGGVVRPGGSLRLSCAASGFTF
DDYGMSWVRQAPGKGLEWVSGINWNGGSTGYADSVKGRFTISRDNAKNSLYLQMNSLRA
EDTAVYYCARGLSLRPWGQGTLVTVSRGGGGSGGGGSGGGGSSELTQDPAVSVALGQTVR
ITCQGDSLRSYYASWYQQKPGQAPVLVIYGKNNRPSGIPDRFSGSSSGNTASLTITGAQAED
EADYYCNSRDSSGNHVVFGGGTKLTVLGAAA
```

### Example 3

### Validation of the SARS-CoV Specific Single-Chain Phage Antibodies

Selected single-chain phage antibodies that were obtained in the screens described above, were validated in ELISA for specificity, i.e. binding to the SARS-CoV preparation prepared as described supra. Additionally, the single-chain phage antibodies were also tested for binding to 10% FBS. For this purpose, the SARS-CoV preparation or 10% FBS preparation was coated to Maxisorp™ ELISA plates. After coating, the plates were blocked in 2% PBS-FM. The selected single-chain phage antibodies were incubated in an equal volume of 4% PBS-FM to obtain blocked phage antibodies. The plates were emptied, washed three times with PBS, after which the blocked phage antibodies were added. Incubation was allowed to proceed for one hour, the plates were washed in PBS containing 0.05% Tween-20 and bound phage antibodies were detected (using OD 492 nm measurement) using an anti-M13 antibody conjugated to peroxidase. As a control, the procedure was performed simultaneously using no single-chain phage antibody or control single-chain phage antibody directed against thyroglobulin (SC02-006) (see De Kruif et al. 1995a and 1995b) or control single-chain phage antibody directed against CD46 (SC02-300). Both controls served as a negative control. As shown in Table 33 the selected phage antibodies called SC03-001, SC03-002, SC03-003, SC03-005, SC03-006, SC03-007, SC03-008, SC03-009, SC03-0010, SC03-012, SC03-013, SC03-014 and SC03-015 displayed significant binding to the immobilized SARS-CoV preparation, while no binding to FBS was observed.

As shown in Table 34 the selected phage antibody called SC03-018 displayed significant binding to the immobilized SARS-CoV preparation, while no binding to FBS was observed. The selected phage antibody called SC03-016 and SC03-017 displayed binding to the immobilized SARS-CoV preparation compared to binding to FBS, although in a lesser amount than SC03-018. The amino acid sequence of SC03-018 is shown in SEQ ID NO:586. The amino acid sequence of the heavy chain CDR3 region of SC03-018 is shown in SEQ ID NO:587.

SC03-018 (SEQ ID NO: 586):
AMAEVQLVESGGGLVQPGGSLRLSCAASGFTF
SSYAMSWVRQAPGKGLEWVSAISGSGGSTYYADSVKGRFTISRDNSKNTLYLQMNSLRAE
DTAVYYCAKFNPFTSFDYWGQGTLVTVSSGGGGSGGGGSGGGGSEIELTQSPSSLSASVGD
RVTITCRASQSISSYLNWYQQKPGKAPKLLIYAASSLQSGVPSRFSGSGSGTDFTLTISSLQPE
DFATYYCQQSYSTPPTFGQGTKVEIKRAAA

Heavy chain CDR3 of SC03-018 (SEQ ID NO:587): FNPFTSFDY

Next, fully human immunoglobulin molecules (human monoclonal anti-SARS-CoV antibodies) were constructed from the selected anti-SARS-CoV single chain Fvs according to standard techniques known to the skilled person in the art. Subsequently, the recombinant human monoclonal antibodies were purified over protein-A columns and size-exclusion columns using standard purification methods used generally for immunoglobulins (see for instance WO 00/63403 which is incorporated by reference herein).

The nucleotide sequence of the heavy chain of the antibody called 03-018 is shown in SEQ ID NO:588. The amino acid sequence of the heavy chain of 03-018 is shown in SEQ ID NO:589. The nucleotide sequence of the light chain of 03-018 is shown in SEQ ID NO:590. The amino acid sequence of 03-018 is shown in SEQ ID NO:591. The amino acid sequence of the heavy chain CDR3 region of 03-018 is shown in SEQ ID NO:587.

Nucleotide sequence of heavy chain of 03-018 (SEQ ID NO: 588):
gag gtg cag ctg gtg gag tct ggg gga ggc ttg gta cag cct ggg ggg
tcc ctg aga ctc tcc tgt gca gcc tct gga ttc acc ttt agc agc tat
gcc atg agc tgg gtc cgc cag gct cca ggg aag ggg ctg gag tgg gtc
tca gct att agt ggt agt ggt ggt agc aca tac tac gca gac tcc gtg
aag ggc cgg ttc acc atc tcc aga gac aat tcc aag aac acg ctg tat
ctg caa atg aac agc ctg aga gcc gag gac acg gcc gtg tat tac tgt
gca aag ttt aat ccg ttt act tcc ttt gac tac tgg ggc cag ggc acc
ctg gtg acc gtc tcc agc gct agc acc aag ggc ccc agc gtg ttc ccc
ctg gcc ccc agc agc aag agc acc agc ggc ggc aca gcc gcc ctg ggc
tgc ctg gtg aag gac tac ttc ccc gag ccc gtg acc gtg agc tgg aac
agc ggc gcc ttg acc agc ggc gtg cac acc ttc ccc gcc gtg ctg cag
agc agc ggc ctg tac agc ctg agc agc gtg gtg acc gtg ccc agc agc
agc ctg ggc acc cag acc tac atc tgc aac gtg aac cac aag ccc agc
aac acc aag gtg gac aaa cgc gtg gag ccc aag agc tgc gac aag acc
cac acc tgc ccc ccc tgc cct gcc ccc gag ctg ctg ggc gga ccc tcc
gtg ttc ctg ttc ccc ccc aag ccc aag gac acc ctc atg atc agc cgg
acc ccc gag gtg acc tgc gtg gtg gtg gac gtg agc cac gag gac ccc
gag gtg aag ttc aac tgg tac gtg gac ggc gtg gag gtg cac aac gcc
aag acc aag ccc cgg gag gag cag tac aac agc acc tac cgg gtg gtg
agc gtg ctc acc gtg ctg cac cag gac tgg ctg aac ggc aag gag tac
aag tgc aag gtg agc aac aag gcc ctg cct gcc ccc atc gag aag acc
atc agc aag gcc aag ggc cag ccc cgg gag ccc cag gtg tac acc ctg
ccc ccc agc cgg gag gag atg acc aag aac cag gtg tcc ctc acc tgt
ctg gtg aag ggc ttc tac ccc agc gac atc gcc gtg gag tgg gag agc
aac ggc cag ccc gag aac aac tac aag acc acc ccc cct gtg ctg gac
agc gac ggc agc ttc ttc ctg tac agc aag ctc acc gtg gac aag agc
cgg tgg cag cag ggc aac gtg ttc agc tgc agc gtg atg cac gag gcc
ctg cac aac cac tac acc cag aag agc ctg agc ctg agc ccc ggc aag
Amino acid sequence of heavy chain of 03-018 (SEQ ID NO: 589):
EVQLVESGGGLVQPGGSLRLSCAASGFTFSSYAMSWVRQAPGKGLEWVSAISGSGGSTYY
ADSVKGRFTISRDNSKNTLYLQMNSLRAEDTAVYYCAKFNPFTSFDYWGQGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSV
MHEALHNHYTQKSLSLSPGK
Nucleotide sequence of light chain of 03-018 (SEQ ID NO: 590):
gac att cag atg acc cag tct cca tcc tcc ctg tct gca tct gta gga
gac aga gtc acc atc act tgc cgg gca agt cag agc att agc agc tac
tta aat tgg tat cag cag aaa cca ggg aaa gcc cct aag ctc ctg atc
tat gct gca tcc agt ttg caa agt ggg gtc cca tca agg ttc agt ggc
agt gga tct ggg aca gat ttc act ctc acc atc agc agt ctg caa cct
gaa gat ttt gca act tac tac tgt caa cag agt tac agt acc cct cca
acg ttc ggc caa ggg acc aag gtg gag atc aaa cgg acc gtg gcc gct
ccc agc gtg ttc atc ttc ccc ccc tcc gac gag cag ctg aag agc ggc
acc gcc agc gtg gtg tgc ctg ctg aac aac ttc tac ccc cgg gag gcc
aag gtg cag tgg aag gtg gac aac gcc ctg cag agc ggc aac agc cag
gag agc gtg acc gag cag gac agc aag gac tcc acc tac agc ctg agc
agc acc ctc acc ctg agc aag gcc gac tac gag aag cac aag gtg tac
gcc tgc gag gtg acc cac cag ggc ctg agc agc ccc gtg acc aag agc
ttc aac cgg ggc gag tgt
Amino acid sequence of light chain of 03-018 (SEQ ID NO: 591):
DIQMTQSPSSLSASVGDRVTITCRASQSISSYLNWYQQKPGKAPKLLIYAASSLQSGVPSRFS
GSGSGTDFTLTISSLQPEDFATYYCQQSYSTPPTFGQGTKVEIKRTVAAPSVFIFPPSDEQLKS
GTASVVCLLNNFYPREAKVQWKVDNALQSGNSQESVTEQDSKDSTYSLSSTLTLSKADYE
KHKVYACEVTHQGLSSPVTKSFNRGEC

### Example 4

### Characterization of Antibody 03-018

To determine which antigen is detected by the human monoclonal anti-SARS-CoV antibody called 03-018, the following sandwich ELISA was performed. For the detection of bound antigens different anti-SARS-CoV rabbit antisera were used. The sandwich ELISA was performed as follows. 03-018 or the control antibody called 02-300 (an antibody against CD46) were immobilized over night at 4° C. to Maxisorp™ ELISA plates at a concentration of 5 μg/ml in coating buffer (50 mM carbonate buffer, pH 9.6). The plates were washed three times with PBS and blocked with PBS containing 1% BSA. Next, a gamma-irradiated SARS-CoV preparation prepared as described herein was denatured by diluting the preparation 1:10 in RIPA buffer (150 mM NaCl, 1% Nonidet P-40, 0.5% deoxycholate, 0.1% sodium dodecyl sulphate, 50 mM Tris, pH 8.0) followed by an incubation of 1 hour at room temperature. Subsequently, the denatured virus preparation was diluted 1:10 in PBS containing 1% BSA and the immobilized human IgGs were incubated with the denatured virus preparation for one hour at room temperature. To recognize which proteins of the SARS-CoV were detected by the immobilized recombinant human monoclonal anti-SARS-CoV antibody polyclonal rabbit antibodies recognizing the complete SARS-CoV, the spike protein of SARS-CoV (Imgenex IMG-542 or IMG-557) or the nucleocapsid protein of SARS-CoV (Imgenex IMG-543) were used. Finally, bound rabbit IgG was detected (using OD 492 nm measurement) using an anti-rabbit-IgG-HRP-conjugate (Dako).

Detection by means of a polyclonal serum against complete SARS-CoV showed that the recombinant human monoclonal anti-SARS-CoV antibody called 03-018 was capable of binding both a native and a denatured SARS-CoV preparation (data not shown). An increased signal after denaturation was observed which might have been caused by the exposure of more antigenic sites upon denaturation. Detection by means of two polyclonal rabbit antibodies against the SARS-CoV spike protein (the antibodies called IMG-542 and IMG-557) or a polyclonal antibody against the SARS-CoV nucleocapsid protein (the antibody called IMG-543) indicated that 03-018 is directed to the nucleocapsid (N) protein of SARS-CoV (data not shown).

Furthermore, wells of ELISA plates were coated overnight with 5 μg/ml anti-myc antibody in 50 mM bicarbonate buffer pH 9.6. The wells of the plates were washed three times with PBS containing 0.05% Tween and blocked for 2 hours at 37° C. with PBS containing 1% BSA. The wells coated with anti-myc antibody were incubated with myc-tagged full length N protein from transfected HEK293T cell lysates diluted in PBS containing 1% BSA for 1 hour at room temperature. The wells were washed three times with PBS containing 0.05% Tween. Next, they were incubated with the above mentioned antibodies. 03-018 bound specifically to the N protein, while not binding the control protein, i.e. bivalent myc-tagged scFv 02-300 (data not shown). Based on the above it was concluded that the recombinant human monoclonal anti-SARS-CoV antibody called 03-018 is directed to the nucleocapsid protein of SARS-CoV.

### Example 5

### Identification of Epitopes Recognized by 03-018 by Means of PEPSCAN-ELISA

PEPSCAN-ELISA was performed essentially as described above. 15-mer linear and looped/cyclic peptides were synthesized from proteins of SARS-CoV and screened using credit-card format mini-PEPSCAN cards (455 peptide formats/card) as described previously (see inter alia WO 84/03564, WO 93/09872, Slootstra et al. 1996). In short, series of overlapping peptides, which were either in linear form or in looped/cyclic form, of all the (potential) proteins of SARS-CoV Urbani, these proteins being called spike protein (the protein-id of the surface spike glycoprotein in the EMBL-database is AAP13441), protein X1 (the protein-id of protein X1 is AAP13446), protein X2 (the protein-id of protein X2 is AAP13447), E protein (the protein-id of the small envelope protein, E protein, is AAP13443), M protein (the protein-id of the membrane protein, M protein, is AAP13444), protein X3 (the protein-id of protein X3 is AAP13448), protein X4 (the protein-id of protein X4 is AAP13449), protein X5 (the protein-id of protein X5 is AAP13450), and N protein (the protein-id of the nucleocapsid protein, N protein, is AAP13445), were produced and tested for binding to the recombinant human anti-SARS-CoV antibody 03-018 (1 μg/ml; diluted in blocking solution which contains 5% horse-serum (v/v) and 5% ovalbumin (w/v)) by means of PEPSCAN analysis.

Because the Urbani proteins indicated above are also found in identical or highly homologous form in other SARS-CoV strains, the antigenic peptides found in the analysis method may not only be used for detection of the SARS-CoV strain Urbani and the prevention and/or treatment of a condition resulting from the SARS-CoV strain Urbani, but may also be useful in detecting SARS-CoV in general and preventing and/or treating a condition resulting from SARS-CoV in general. The accession number in the EMBL-database of the complete genome of the strains TOR2, Frankfurt 1 and HSR 1 is AY274119, AY291315 and AY323977, respectively. Under these accession numbers the amino acid sequence of the other (potential) proteins of these strains can be found.

Particularly interesting appear to be domains comprising several relevant peptides. These domains are indicated (colored grey) in Table 35. The recombinant human anti-SARS-CoV antibody called 03-018 specifically reacted with peptides of the nucleocapsid (N) protein. The peptides recognized include NGPQSNQRSAPRITF (SEQ ID NO:592), GPQSNQRSAPRITFG (SEQ ID NO:593), PQSNQRSAPRITFGG (SEQ ID NO:594), QSNQRSAPRITFGGP (SEQ ID NO:595), SNQRSAPRITFGGPT (SEQ ID NO:596), NQRSAPRITFGGPTD (SEQ ID NO:597), QRSAPRITFGGPTDS (SEQ ID NO:598), RSAPRITFGGPTDST (SEQ ID NO:599), SAPRITFGGPTDSTD (SEQ ID NO:600), APRITFGGPTDSTDN (SEQ ID NO:601), PRITFGGPTDSTDNN (SEQ ID NO:602), RITFGGPTDSTDNNQ (SEQ ID NO:603) and ITFGGPTDSTDNNQN (SEQ ID NO:604). Highest binding of 03-018 was found with a continuous series of linear and looped peptides, starting with the sequence GPQSNQRSAPRITFG (SEQ ID NO:593) and ending with the peptide RSAPRITFGGPTDST (SEQ ID NO:599), thereby having the minimal sequence RSAPRITFG (SEQ ID NO:605) in common. The peptides NGPQSNQRSAPRITF (SEQ ID NO:592), GPQSNQRSAPRITFG (SEQ ID NO:593), PQSNQRSAPRITFGG (SEQ ID NO:594) and QSNQRSAPRITFGGP (SEQ ID NO:595) were also recognized by antibodies from a rabbit serum derived from a rabbit that has been immunized with SARS-CoV strain Frankfurt 1 (see Table 32). Through the above approach, the minimal binding site of 03-018 was mapped to residues 11-19 of the N protein, which corresponds with the sequence RSAPRITFG. Interestingly, this linear epitope is conserved in the N protein sequence of all published human SARS-CoV and animal SARS-CoV-like isolates but is absent in other members of the family of Coronaviridae. This suggests that the peptides found, in particular the ones having the minimal binding site of 03-018 are useful in the prevention, treatment and/or detection of SARS-CoV in general.

TABLE 1
Binding of the sera called SARS-yellow, SARS-green, 1a,
1b, 2, 6, 37, 62 and London to linear peptides of protein X1 of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MDLFMRFFTLGSITA | 0.8 | 0.7 | 0.7 | 0.5 | 0.7 | 0.7 | 0.5 | 0.6 | 0.3 | 607 |
| DLFMRFFTLGSITAQ | 0.7 | 0.5 | 0.4 | 0.1 | 0.6 | 0.5 | 0.4 | 0.2 | 0.2 | 608 |
| LFMRFFTLGSITAQP | 0.8 | 0.7 | 0.6 | 0.5 | 0.6 | 0.6 | 0.6 | 0.2 | 0.3 | 609 |
| FMRFFTLGSITAQPV | 0.8 | 0.6 | 0.8 | 0.5 | 0.7 | 0.7 | 0.7 | 0.3 | 0.3 | 610 |
| MRFFTLGSITAQPVK | 0.6 | 0.4 | 0.4 | 0.6 | 0.6 | 0.4 | 0.4 | 0.2 | 0.5 | 611 |
| 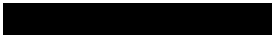 | 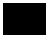 | 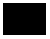 | 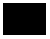 | 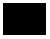 | 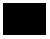 | 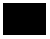 | 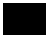 | 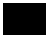 | 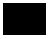 | 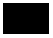 |
| 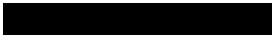 | 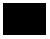 | 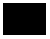 | 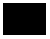 | 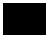 | 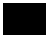 | 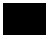 | 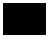 | 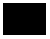 | 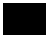 | 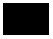 |
| 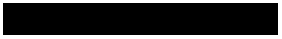 | 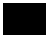 | 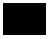 | 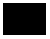 | 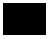 | 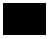 | 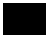 | 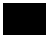 | 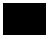 | 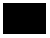 | 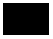 |
| 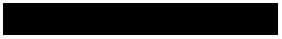 | 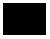 | 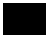 | 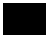 | 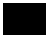 | 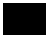 | 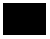 | 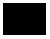 | 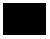 | 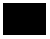 | 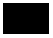 |
| 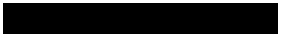 | 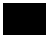 | 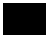 | 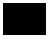 | 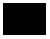 | 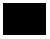 | 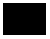 | 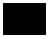 | 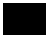 | 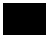 | 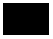 |
| 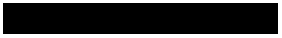 | 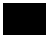 | 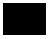 | 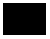 | 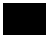 | 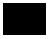 | 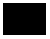 | 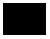 | 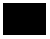 | 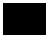 | 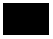 |
| 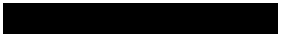 | 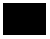 | 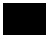 | 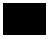 | 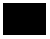 | 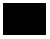 | 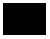 | 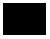 | 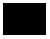 | 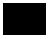 | 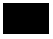 |
| 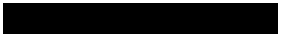 | 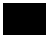 | 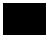 | 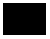 | 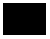 | 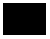 | 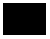 | 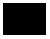 | 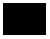 | 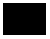 | 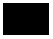 |
| 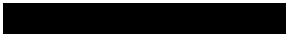 | 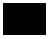 | 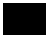 | 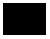 | 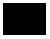 | 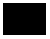 | 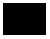 | 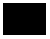 | 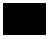 | 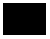 | 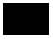 |
| 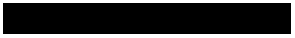 | 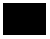 | 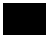 | 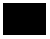 | 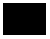 | 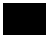 | 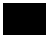 | 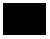 | 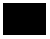 | 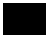 | 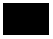 |
| 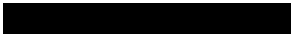 | 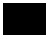 | 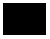 | 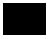 | 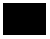 | 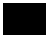 | 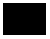 | 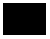 | 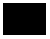 | 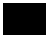 | 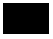 |
| 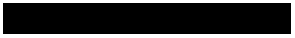 | 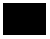 | 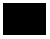 | 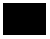 | 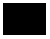 | 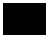 | 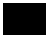 | 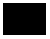 | 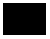 | 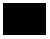 | 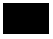 |
| 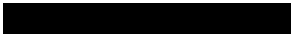 | 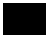 | 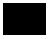 | 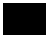 | 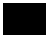 | 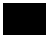 | 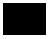 | 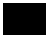 | 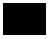 | 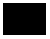 | 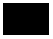 |
| 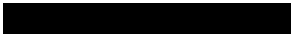 | 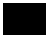 | 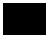 | 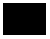 | 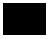 | 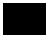 | 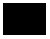 | 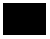 | 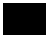 | 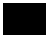 | 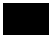 |
| 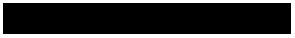 | 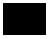 | 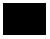 | 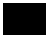 | 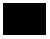 | 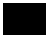 | 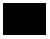 | 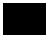 | 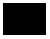 | 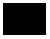 | 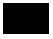 |
| 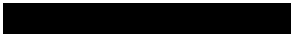 | 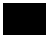 | 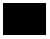 | 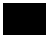 | 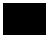 | 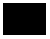 | 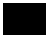 | 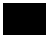 | 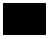 | 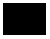 | 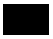 |
| 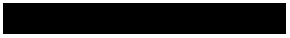 | 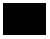 | 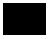 | 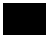 | 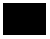 | 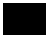 | 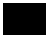 | 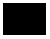 | 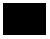 | 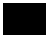 | 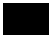 |
| 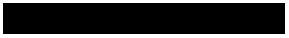 | 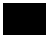 | 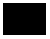 | 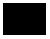 | 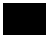 | 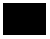 | 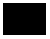 | 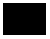 | 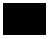 | 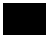 | 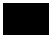 |
| 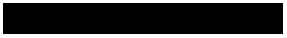 | 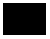 | 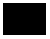 | 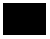 | 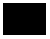 | 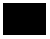 | 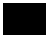 | 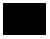 | 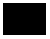 | 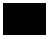 | 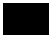 |
| 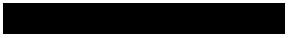 | 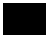 | 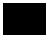 | 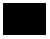 | 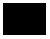 | 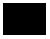 | 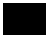 | 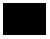 | 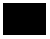 | 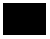 | 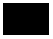 |
| 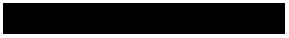 | 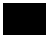 | 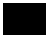 | 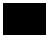 | 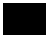 | 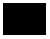 | 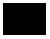 | 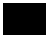 | 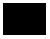 | 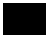 | 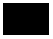 |
| 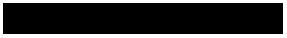 | 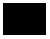 | 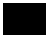 | 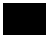 | 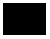 | 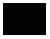 | 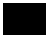 | 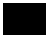 | 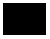 | 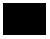 | 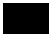 |
| 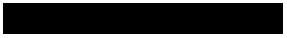 | 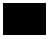 | 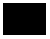 | 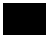 | 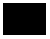 | 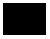 | 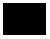 | 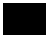 | 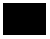 | 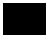 | 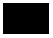 |
| 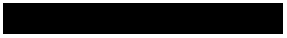 | 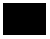 | 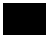 | 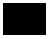 | 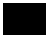 | 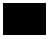 | 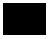 | 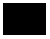 | 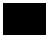 | 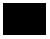 | 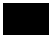 |
| HATATIPLQASLPFG | 0.9 | 0.8 | 0.6 | 0.6 | 0.8 | 0.7 | 0.4 | 0.2 | 0.3 | 612 |
| ATATIPLQASLPFGW | 0.9 | 0.7 | 0.7 | 0.7 | 0.7 | 0.8 | 0.5 | 0.4 | 0.4 | 613 |
| TATIPLQASLPFGWL | 0.9 | 0.8 | 0.6 | 0.7 | 0.7 | 0.7 | 0.3 | 0.5 | 0.3 | 614 |
| ATIPLQASLPFGWLV | 0.7 | 0.5 | 0.6 | 0.6 | 0.7 | 0.7 | 0.3 | 0.2 | 0.3 | 615 |
| TIPLQASLPFGWLVI | 0.8 | 0.5 | 0.6 | 0.5 | 0.7 | 0.8 | 0.4 | 0.4 | 0.3 | 616 |
| IPLQASLPFGWLVIG | 0.8 | 0.6 | 0.6 | 0.5 | 0.7 | 0.6 | 0.5 | 0.4 | 0.3 | 617 |
| PLQASLPFGWLVIGV | 0.8 | 0.5 | 0.5 | 0.5 | 0.7 | 0.7 | 0.6 | 0.4 | 0.3 | 618 |
| LQASLPFGWLVIGVA | 0.7 | 0.6 | 0.6 | 0.4 | 0.7 | 0.7 | 0.6 | 0.3 | 0.2 | 619 |
| QASLPFGWLVIGVAF | 0.7 | 0.6 | 0.6 | 0.4 | 0.6 | 0.6 | 0.5 | 0.1 | 0.2 | 620 |
| ASLPFGWLVIGVAFL | 0.7 | 0.5 | 0.6 | 0.5 | 0.6 | 0.6 | 0.4 | 0.2 | 0.3 | 621 |
| SLPFGWLVIGVAFLA | 0.7 | 0.6 | 0.5 | 0.4 | 0.6 | 0.5 | 0.4 | 0.2 | 0.2 | 622 |
| LPFGWLVIGVAFLAV | 0.8 | 0.6 | 0.7 | 0.5 | 0.7 | 0.8 | 0.7 | 0.3 | 0.3 | 623 |
| PFGWLVIGVAFLAVF | 0.7 | 0.5 | 0.5 | 0.4 | 0.7 | 0.6 | 0.7 | 0.3 | 0.3 | 624 |
| FGWLVIGVAFLAVFQ | 0.7 | 0.5 | 0.5 | 0.4 | 0.6 | 0.6 | 0.5 | 0.1 | 0.3 | 625 |
| GWLVIGVAFLAVFQS | 0.7 | 0.6 | 0.5 | 0.5 | 0.6 | 0.6 | 0.5 | 0.2 | 0.3 | 626 |
| WLVIGVAFLAVFQSA | 0.6 | 0.5 | 0.5 | 0.4 | 0.7 | 0.6 | 0.4 | 0.3 | 0.3 | 627 |
| LVIGVAFLAVFQSAT | 0.7 | 0.6 | 0.6 | 0.4 | 0.6 | 0.6 | 0.4 | 0.3 | 0.3 | 628 |
| VIGVAFLAVFQSATK | 0.5 | 0.4 | 0.4 | 0.4 | 0.5 | 0.4 | 0.2 | 0.1 | 0.2 | 629 |
| IGVAFLAVFQSATKI | 0.6 | 0.5 | 0.6 | 0.5 | 0.6 | 0.6 | 0.3 | 0.2 | 0.2 | 630 |
| GVAFLAVFQSATKII | 0.7 | 0.6 | 0.8 | 0.7 | 0.6 | 0.6 | 0.2 | 0.3 | 0.2 | 631 |
| VAFLAVFQSATKIIA | 0.6 | 0.5 | 0.5 | 0.7 | 0.6 | 0.6 | 0.4 | 0.2 | 0.2 | 632 |
| AFLAVFQSATKIIAL | 0.7 | 0.4 | 0.5 | 0.4 | 0.6 | 0.5 | 0.3 | 0.3 | 0.2 | 633 |
| FLAVFQSATKIIALN | 0.6 | 0.4 | 0.5 | 0.4 | 0.6 | 0.6 | 0.4 | 0.3 | 0.2 | 634 |
| LAVFQSATKIIALNK | 0.7 | 0.5 | 0.5 | 0.6 | 0.6 | 0.5 | 0.4 | 0.3 | 0.2 | 635 |
| AVFQSATKIIALNKR | 0.8 | 0.6 | 0.6 | 0.8 | 0.7 | 0.6 | 0.5 | 0.3 | 0.3 | 492 |
| VFQSATKIIALNKRW | 0.8 | 0.6 | 0.6 | 0.6 | 0.7 | 0.8 | 0.5 | 0.2 | 0.3 | 493 |
| FQSATKIIALNKRWQ | 0.8 | 0.6 | 0.6 | 0.6 | 0.7 | 0.7 | 0.4 | 0.4 | 0.3 | 494 |
| QSATKIIALNKRWQL | 0.8 | 0.7 | 0.7 | 0.7 | 0.7 | 0.8 | 0.5 | 0.4 | 0.4 | 495 |
| SATKIIALNKRWQLA | 0.8 | 0.6 | 0.6 | 0.7 | 0.7 | 0.8 | 0.5 | 0.3 | 0.4 | 496 |
| ATKIIALNKRWQLAL | 0.7 | 0.6 | 0.6 | 0.7 | 0.8 | 0.8 | 0.7 | 0.3 | 0.4 | 497 |
| TKIIALNKRWQLALY | 0.7 | 0.5 | 0.6 | 1.0 | 0.7 | 0.7 | 0.5 | 0.2 | 0.3 | 498 |
| KIIALNKRWQLALYK | 0.8 | 0.7 | 0.7 | 1.0 | 0.8 | 0.8 | 0.5 | 0.4 | 0.4 | 499 |
| IIALNKRWQLALYKG | 0.7 | 0.4 | 0.5 | 0.7 | 0.6 | 0.5 | 0.5 | 0.3 | 0.3 | 500 |
| IALNKRWQLALYKGF | 0.8 | 0.7 | 0.6 | 0.9 | 0.7 | 0.7 | 0.5 | 0.4 | 0.3 | 501 |
| ALNKRWQLALYKGFQ | 0.7 | 0.6 | 0.5 | 0.5 | 0.6 | 0.5 | 0.4 | 0.2 | 0.2 | 502 |
| LNKRWQLALYKGFQF | 0.6 | 0.7 | 0.8 | 0.8 | 0.6 | 0.6 | 0.3 | 0.3 | 0.3 | 503 |
| NKRWQLALYKGFQFI | 0.7 | 0.5 | 0.7 | 0.8 | 0.6 | 0.6 | 0.3 | 0.3 | 0.3 | 504 |
| KRWQLALYKGFQFIC | 0.6 | 0.5 | 0.5 | 0.6 | 0.6 | 0.5 | 0.2 | 0.1 | 0.2 | 636 |
| RWQLALYKGFQFICN | 0.7 | 0.5 | 0.5 | 0.4 | 0.6 | 0.5 | 0.2 | 0.3 | 0.3 | 637 |
| WQLALYKGFQFICNL | 0.6 | 0.2 | 0.4 | 0.5 | 0.3 | 0.4 | 0.4 | 0.3 | 0.2 | 638 |
| QLALYKGFQPICNLL | 0.6 | 0.5 | 0.4 | 0.5 | 0.6 | 0.6 | 0.5 | 0.2 | 0.2 | 639 |
| LALYKGFQFICNLLL | 0.7 | 0.5 | 0.5 | 0.4 | 0.5 | 0.6 | 0.5 | 0.2 | 0.2 | 640 |
| ALYKGFQFICNLLLL | 0.6 | 0.5 | 0.4 | 0.4 | 0.5 | 0.5 | 0.5 | 0.2 | 0.2 | 641 |
| LYKGFQFICNLLLLF | 0.6 | 0.5 | 0.5 | 0.4 | 0.5 | 0.4 | 0.4 | 0.2 | 0.2 | 642 |
| YKGFQFICNLLLLFV | 0.9 | 0.8 | 0.9 | 0.9 | 0.7 | 1.0 | 0.6 | 0.4 | 0.8 | 643 |
| KGFQFICNLLLLFVT | 0.6 | 0.5 | 0.6 | 0.5 | 0.6 | 0.7 | 0.6 | 0.2 | 0.3 | 644 |
| GFQFICNLLLLFVTI | 0.6 | 0.5 | 0.5 | 0.4 | 0.5 | 0.6 | 0.5 | 0.2 | 0.3 | 645 |
| FQFICNLLLLFVTIY | 0.6 | 0.5 | 0.5 | 0.3 | 0.5 | 0.5 | 0.5 | 0.1 | 0.2 | 646 |
| QFICNLLLLFVTIYS | 0.6 | 0.5 | 0.6 | 0.4 | 0.5 | 0.6 | 0.4 | 0.0 | 0.2 | 647 |
| FICNLLLLFVTIYSH | 0.6 | 0.6 | 0.6 | 0.4 | 0.5 | 0.5 | 0.4 | 0.2 | 0.3 | 648 |
| ICNLLLLFVTIYSHL | 0.6 | 0.6 | 0.5 | 0.4 | 0.6 | 0.5 | 0.4 | 0.3 | 0.2 | 649 |
| CNLLLLFVTIYSHLL | 0.7 | 0.5 | 0.5 | 0.4 | 0.6 | 0.4 | 0.4 | 0.1 | 0.2 | 650 |
| NLLLLFVTIYSHLLL | 0.7 | 0.5 | 0.5 | 0.4 | 0.5 | 0.4 | 0.3 | 0.1 | 0.2 | 651 |
| LLLLFVTIYSHLLLV | 0.7 | 0.5 | 0.8 | 0.4 | 0.5 | 0.4 | 0.3 | 0.3 | 0.2 | 652 |
| LLLFVTIYSHLLLVA | 0.7 | 0.5 | 0.6 | 0.3 | 0.5 | 0.4 | 0.3 | 0.0 | 0.2 | 653 |
| LLFVTIYSHLLLVAA | 0.7 | 0.2 | 0.5 | 0.4 | 0.6 | 0.5 | 0.3 | 0.3 | 0.2 | 654 |
| LFVTIYSHLLLVAAG | 0.7 | 0.4 | 0.6 | 0.3 | 0.6 | 0.5 | 0.5 | 0.2 | 0.2 | 655 |
| FVTIYSHLLLVAAGM | 0.7 | 0.5 | 0.6 | 0.4 | 0.5 | 0.5 | 0.5 | 0.3 | 0.2 | 656 |
| VTIYSHLLLVAAGME | 0.8 | 0.7 | 0.5 | 0.4 | 0.6 | 0.6 | 0.6 | 0.3 | 0.3 | 657 |
| TIYSHLLLVAAGMEA | 0.6 | 0.5 | 0.4 | 0.3 | 0.5 | 0.4 | 0.5 | 0.3 | 0.2 | 658 |
| IYSHLLLVAAGMEAQ | 0.6 | 0.5 | 0.5 | 0.4 | 0.6 | 0.6 | 0.3 | 0.2 | 0.3 | 659 |
| YSHLLLVAAGMEAQF | 0.7 | 0.6 | 0.5 | 0.5 | 0.6 | 0.7 | 0.4 | 0.3 | 0.3 | 660 |
| SHLLLVAAGMEAQFL | 0.8 | 0.7 | 0.7 | 0.6 | 0.8 | 0.8 | 0.7 | 0.3 | 0.3 | 661 |
| HLLLVAAGMEAQFLY | 0.9 | 0.7 | 0.6 | 0.5 | 0.7 | 0.7 | 0.6 | 0.2 | 0.3 | 662 |
| LLLVAAGMEAQFLYL | 0.9 | 0.8 | 0.6 | 0.5 | 0.6 | 0.6 | 0.6 | 0.1 | 0.2 | 663 |
| LLVAAGMEAQFLYLY | 0.8 | 0.7 | 0.6 | 0.5 | 0.6 | 0.5 | 0.4 | 0.1 | 0.3 | 664 |
| LVAAGMEAQFLYLYA | 0.8 | 0.7 | 0.6 | 0.4 | 0.6 | 0.5 | 0.3 | 0.2 | 0.2 | 665 |
| VAAGMEAQFLYLYAL | 0.7 | 0.6 | 0.5 | 0.4 | 0.5 | 0.4 | 0.3 | 0.2 | 0.2 | 666 |
| AAGMEAQFLYLYALI | 0.7 | 0.6 | 0.6 | 0.5 | 0.5 | 0.5 | 0.3 | 0.2 | 0.2 | 667 |
| AGMEAQFLYLYALIY | 0.7 | 0.6 | 0.6 | 0.4 | 0.5 | 0.4 | 0.2 | 0.1 | 0.2 | 668 |
| GMEAQFLYLYALIYF | 0.8 | 0.6 | 0.6 | 0.5 | 0.5 | 0.4 | 0.3 | 0.1 | 0.2 | 669 |
| MEAQFLYLYALIYFL | 0.7 | 0.6 | 0.5 | 0.4 | 0.5 | 0.4 | 0.2 | 0.0 | 0.2 | 670 |
| EAQFLYLYALIYFLQ | 0.7 | 0.4 | 0.6 | 0.4 | 0.5 | 0.4 | 0.2 | 0.1 | 0.2 | 671 |
| AQFLYLYALIYFLQC | 0.6 | 0.5 | 0.5 | 0.3 | 0.6 | 0.4 | 0.4 | 0.2 | 0.2 | 672 |
| QFLYLYALIYFLQCI | 0.7 | 0.5 | 0.5 | 0.4 | 0.6 | 0.5 | 0.5 | 0.2 | 0.2 | 673 |
| FLYLYALIYFLQCIN | 0.7 | 0.5 | 0.5 | 0.4 | 0.5 | 0.6 | 0.5 | 0.2 | 0.2 | 674 |
| LYLYALIYFLQCINA | 0.7 | 0.5 | 0.4 | 0.3 | 0.5 | 0.5 | 0.5 | 0.2 | 0.2 | 675 |
| YLYALIYFLQCINAC | 0.7 | 0.5 | 0.5 | 0.5 | 0.6 | 0.5 | 0.3 | 0.2 | 0.4 | 676 |
| LYALIYFLQCINACR | 0.7 | 0.5 | 0.5 | 0.5 | 0.6 | 0.6 | 0.4 | 0.1 | 0.3 | 677 |
| YALIYFLQCINACRI | 0.7 | 0.5 | 0.5 | 0.4 | 0.6 | 0.6 | 0.4 | 0.2 | 0.3 | 678 |
| ALIYFLQCINACRII | 0.6 | 0.6 | 0.5 | 0.4 | 0.7 | 0.6 | 0.4 | 0.1 | 0.3 | 679 |
| LIYFLQCINACRIIM | 0.7 | 0.6 | 0.6 | 0.5 | 0.6 | 0.6 | 0.4 | 0.2 | 0.3 | 680 |
| IYFLQCINACRIIMR | 0.7 | 0.6 | 0.6 | 0.5 | 0.7 | 0.7 | 0.5 | 0.2 | 0.3 | 681 |
| YFLQCINACRIIMRC | 0.7 | 0.6 | 0.5 | 0.5 | 0.6 | 0.6 | 0.3 | 0.3 | 0.2 | 682 |
| FLQCINACRIIMRCW | 0.8 | 0.6 | 0.6 | 0.7 | 0.7 | 0.6 | 0.4 | 0.3 | 0.3 | 683 |
| LQCINACRIIMRCWL | 0.7 | 0.5 | 0.5 | 0.6 | 0.7 | 0.6 | 0.3 | 0.1 | 0.3 | 505 |
| QCINACRIIMRCWLC | 0.8 | 0.6 | 0.5 | 0.8 | 0.7 | 0.7 | 0.3 | 0.2 | 0.4 | 506 |
| CINACRIIMRCWLCW | 0.8 | 0.5 | 0.5 | 0.7 | 0.6 | 0.7 | 0.4 | 0.2 | 0.4 | 507 |
| 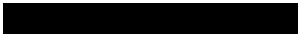 | 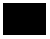 | 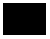 | 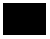 | 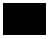 | 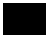 | 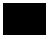 | 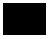 | 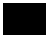 | 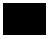 | 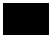 |
| 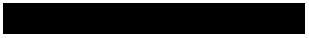 | 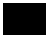 | 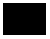 | 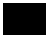 | 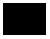 | 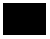 | 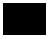 | 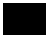 | 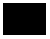 | 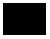 | 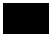 |
| 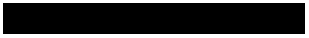 | 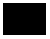 | 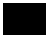 | 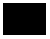 | 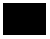 | 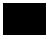 | 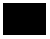 | 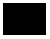 | 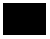 | 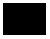 | 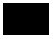 |
| 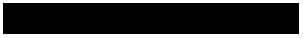 | 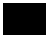 | 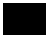 | 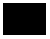 | 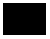 | 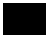 | 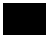 | 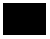 | 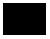 | 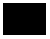 | 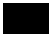 |
| 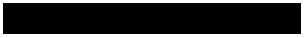 | 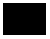 | 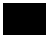 | 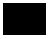 | 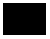 | 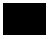 | 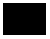 | 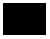 | 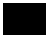 | 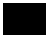 | 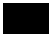 |
| 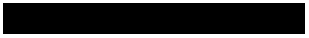 | 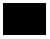 | 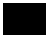 | 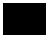 | 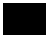 | 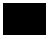 | 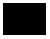 | 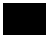 | 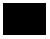 | 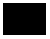 | 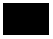 |
| 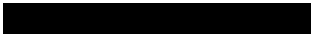 | 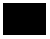 | 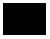 | 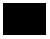 | 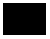 | 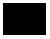 | 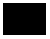 | 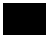 | 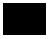 | 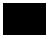 | 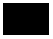 |
| 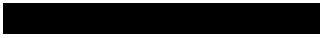 | 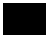 | 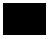 | 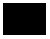 | 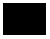 | 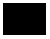 | 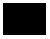 | 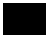 | 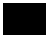 | 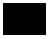 | 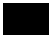 |
| 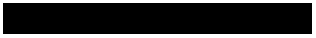 | 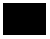 | 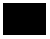 | 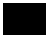 | 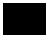 | 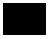 | 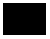 | 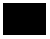 | 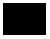 | 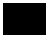 | 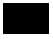 |
| 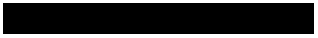 | 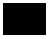 | 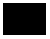 | 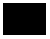 | 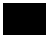 | 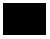 | 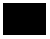 | 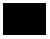 | 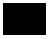 | 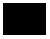 | 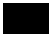 |
| 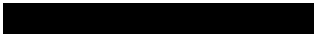 | 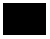 | 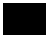 | 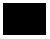 | 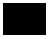 | 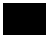 | 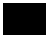 | 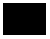 | 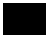 | 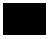 | 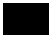 |
| 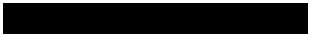 | 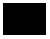 | 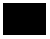 | 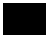 | 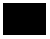 | 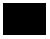 | 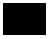 | 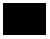 | 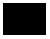 | 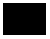 | 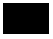 |
| 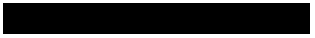 | 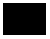 | 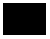 | 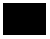 | 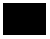 | 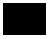 | 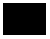 | 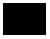 | 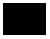 | 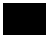 | 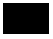 |
| WKCKSKNPLLYDANY | 0.8 | 0.8 | 0.6 | 0.7 | 0.8 | 0.7 | 0.5 | 0.4 | 0.3 | 684 |
| KCKSKNPLLYDANYF | 0.7 | 0.4 | 0.5 | 0.5 | 0.6 | 0.5 | 0.4 | 0.2 | 0.2 | 685 |
| CKSKNPLLYDANYFV | 0.8 | 0.5 | 0.5 | 0.6 | 0.7 | 0.5 | 0.2 | 0.2 | 0.3 | 686 |
| KSKNPLLYDANYFVC | 0.7 | 0.4 | 0.4 | 0.4 | 0.5 | 0.4 | 0.3 | 0.2 | 0.2 | 687 |
| SKNPLLYDANYFVCW | 0.7 | 0.5 | 0.4 | 0.5 | 0.5 | 0.4 | 0.4 | 0.2 | 0.2 | 688 |
| KNPLLYDANYFVCWH | 0.8 | 0.5 | 0.5 | 0.4 | 0.6 | 0.5 | 0.3 | 0.4 | 0.3 | 689 |
| NPLLYDANYFVCWHT | 0.9 | 0.6 | 0.6 | 0.5 | 0.8 | 0.7 | 0.4 | 0.4 | 0.3 | 690 |
| PLLYDANYFVCWHTH | 0.9 | 0.8 | 0.6 | 0.6 | 0.8 | 0.8 | 0.5 | 0.4 | 0.4 | 691 |
| LLYDANYFVCWHTHN | 0.9 | 0.7 | 0.6 | 0.7 | 0.7 | 0.8 | 0.5 | 0.4 | 0.4 | 692 |
| LYDANYFVCWHTHNY | 0.9 | 0.8 | 0.5 | 0.7 | 0.8 | 0.8 | 0.5 | 0.4 | 0.4 | 693 |
| 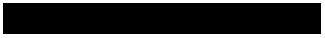 | 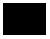 | 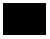 | 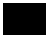 | 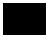 | 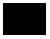 | 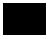 | 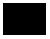 | 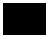 | 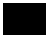 | 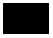 |
| 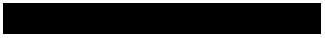 | 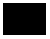 | 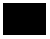 | 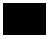 | 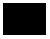 | 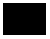 | 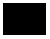 | 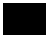 | 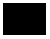 | 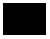 | 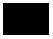 |
| 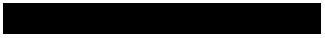 | 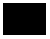 | 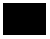 | 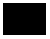 | 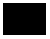 | 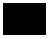 | 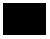 | 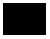 | 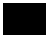 | 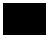 | 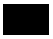 |
| 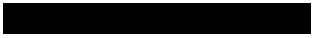 | 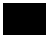 | 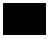 | 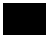 | 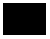 | 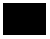 | 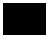 | 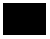 | 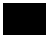 | 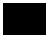 | 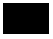 |
| 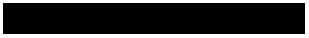 | 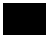 | 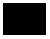 | 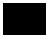 | 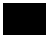 | 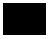 | 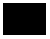 | 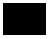 | 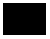 | 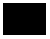 | 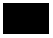 |
| 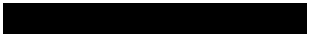 | 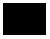 | 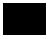 | 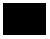 | 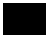 | 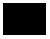 | 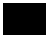 | 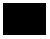 | 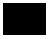 | 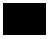 | 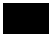 |
| 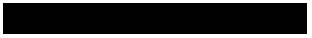 | 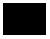 | 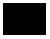 | 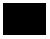 | 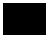 | 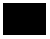 | 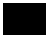 | 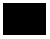 | 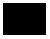 | 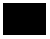 | 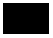 |
| 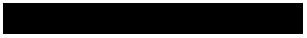 | 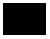 | 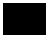 | 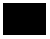 | 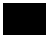 | 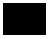 | 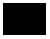 | 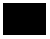 | 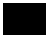 | 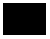 | 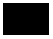 |
| 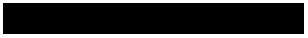 | 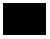 | 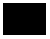 | 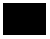 | 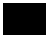 | 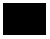 | 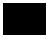 | 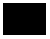 | 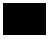 | 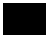 | 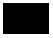 |
| 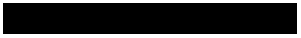 | 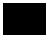 | 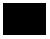 | 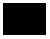 | 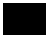 | 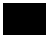 | 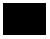 | 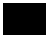 | 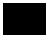 | 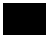 | 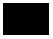 |
| 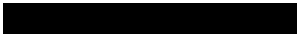 | 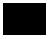 | 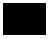 | 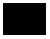 | 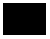 | 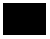 | 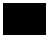 | 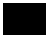 | 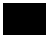 | 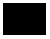 | 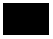 |
| 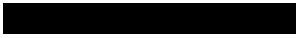 | 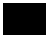 | 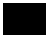 | 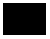 | 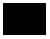 | 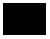 | 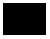 | 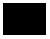 | 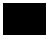 | 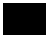 | 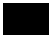 |
| 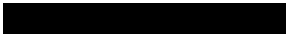 | 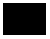 | 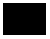 | 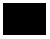 | 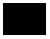 | 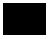 | 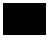 | 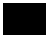 | 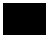 | 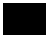 | 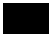 |
| 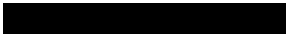 | 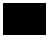 | 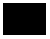 | 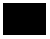 | 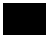 | 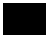 | 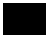 | 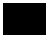 | 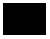 | 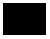 | 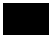 |
| 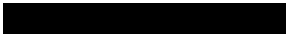 | 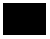 | 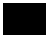 | 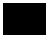 | 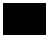 | 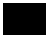 | 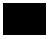 | 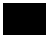 | 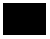 | 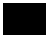 | 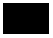 |
| 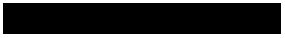 | 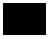 | 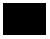 | 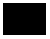 | 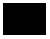 | 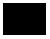 | 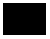 | 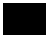 | 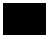 | 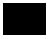 | 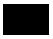 |
| CIPYNSVTDTIVVTE | 0.7 | 0.6 | 0.5 | 0.5 | 0.7 | 0.6 | 0.5 | 0.3 | 0.3 | 694 |
| IPYNSVTDTIVVTEG | 0.7 | 0.5 | 0.5 | 0.4 | 0.6 | 0.4 | 0.4 | 0.3 | 0.2 | 695 |
| PYNSVTDTIVVTEGD | 0.5 | 0.4 | 0.4 | 0.4 | 0.5 | 0.4 | 0.3 | 0.2 | 0.2 | 696 |
| YNSVTDTIVVTEGDG | 0.6 | 0.5 | 0.4 | 0.4 | 0.5 | 0.5 | 0.4 | 0.3 | 0.2 | 697 |
| NSVTDTIVVTEGDGI | 0.6 | 0.5 | 0.4 | 0.4 | 0.5 | 0.4 | 0.6 | 0.2 | 0.2 | 698 |
| SVTDTIVVTEGDGIS | 0.6 | 0.5 | 0.4 | 0.4 | 0.5 | 0.5 | 0.4 | 0.1 | 0.2 | 699 |
| VTDTIVVTEGDGIST | 0.6 | 0.5 | 0.3 | 0.4 | 0.5 | 0.4 | 0.3 | 0.1 | 0.2 | 700 |
| TDTIVVTEGDGISTP | 0.6 | 0.5 | 0.4 | 0.5 | 0.5 | 0.4 | 0.2 | 0.3 | 0.2 | 701 |
| DTIVVTEGDGISTPK | 0.5 | 0.4 | 0.3 | 0.4 | 0.4 | 0.3 | 0.1 | 0.2 | 0.2 | 702 |
| TIVVTEGDGISTPKL | 0.6 | 0.5 | 0.5 | 0.6 | 0.6 | 0.5 | 0.3 | 0.3 | 0.3 | 703 |
| IVVTEGDGISTPKLK | 0.5 | 0.5 | 0.3 | 0.4 | 0.5 | 0.3 | 0.1 | 0.1 | 0.2 | 704 |
| VVTEGDGISTPKLKE | 0.5 | 0.4 | 0.3 | 0.2 | 0.4 | 0.3 | 0.1 | 0.1 | 0.3 | 705 |
| VTEGDGISTPKLKED | 0.5 | 0.4 | 0.3 | 0.3 | 0.4 | 0.3 | 0.2 | 0.1 | 0.3 | 706 |
| TEGDGISTPKLKEDY | 0.5 | 0.3 | 0.4 | 0.2 | 0.4 | 0.3 | 0.0 | 0.1 | 0.2 | 707 |
| EGDGISTPKLKEDYQ | 0.6 | 0.3 | 0.6 | 0.3 | 0.5 | 0.4 | 0.2 | 0.2 | 0.3 | 708 |
| 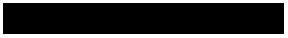 | 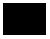 | 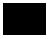 | 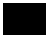 | 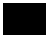 | 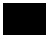 | 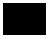 | 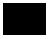 | 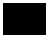 | 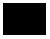 | 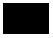 |
| 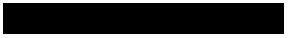 | 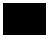 | 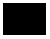 | 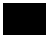 | 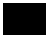 | 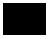 | 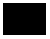 | 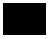 | 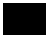 | 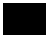 | 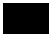 |
| 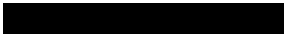 | 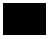 | 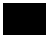 | 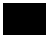 | 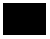 | 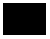 | 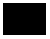 | 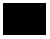 | 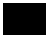 | 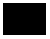 | 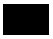 |
| 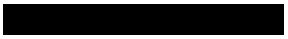 | 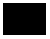 | 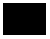 | 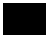 | 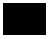 | 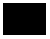 | 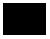 | 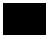 | 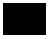 | 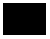 | 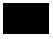 |
| 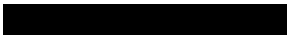 | 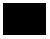 | 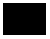 | 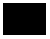 | 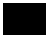 | 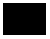 | 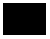 | 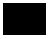 | 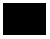 | 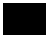 | 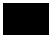 |
| 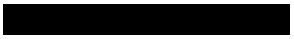 | 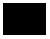 | 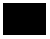 | 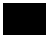 | 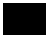 | 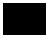 | 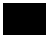 | 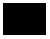 | 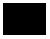 | 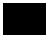 | 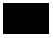 |
| 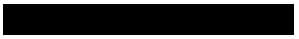 | 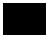 | 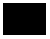 | 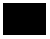 | 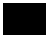 | 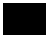 | 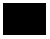 | 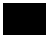 | 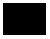 | 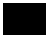 | 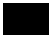 |
| 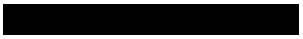 | 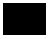 | 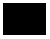 | 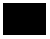 | 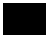 | 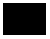 | 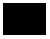 | 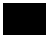 | 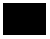 | 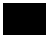 | 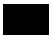 |
| 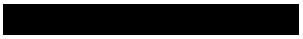 | 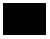 | 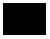 | 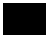 | 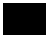 | 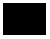 | 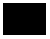 | 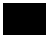 | 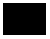 | 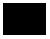 | 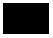 |
| 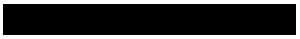 | 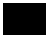 | 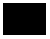 | 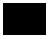 | 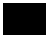 | 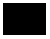 | 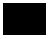 | 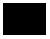 | 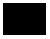 | 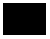 | 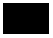 |
| 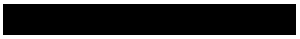 | 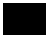 | 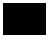 | 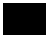 | 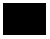 | 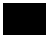 | 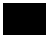 | 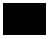 | 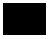 | 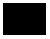 | 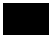 |
| 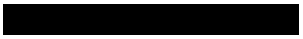 | 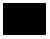 | 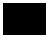 | 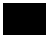 | 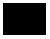 | 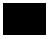 | 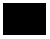 | 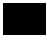 | 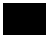 | 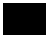 | 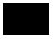 |
| 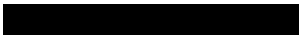 | 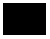 | 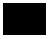 | 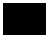 | 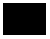 | 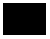 | 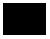 | 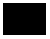 | 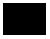 | 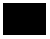 | 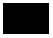 |
| 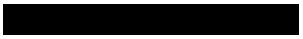 | 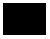 | 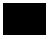 | 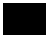 | 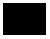 | 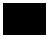 | 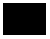 | 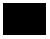 | 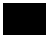 | 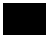 | 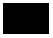 |
| 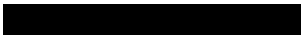 | 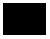 | 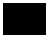 | 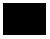 | 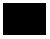 | 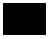 | 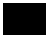 | 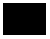 | 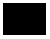 | 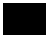 | 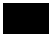 |
| 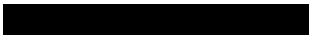 | 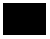 | 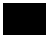 | 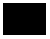 | 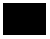 | 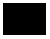 | 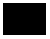 | 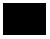 | 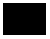 | 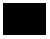 | 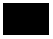 |
| 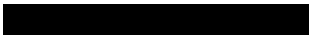 | 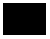 | 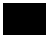 | 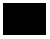 | 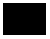 | 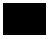 | 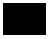 | 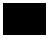 | 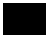 | 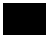 | 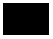 |
| 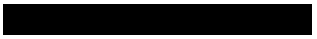 | 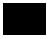 | 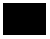 | 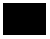 | 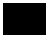 | 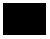 | 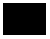 | 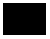 | 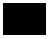 | 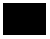 | 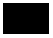 |
| 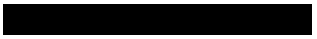 | 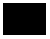 | 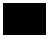 | 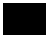 | 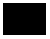 | 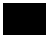 | 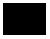 | 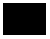 | 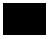 | 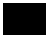 | 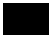 |
| 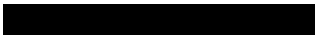 | 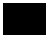 | 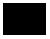 | 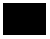 | 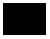 | 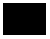 | 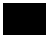 | 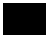 | 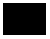 | 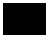 | 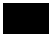 |
| 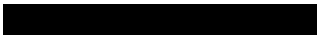 | 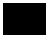 | 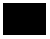 | 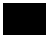 | 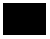 | 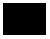 | 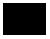 | 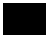 | 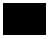 | 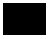 | 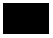 |
| 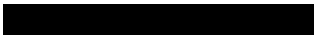 | 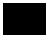 | 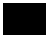 | 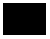 | 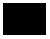 | 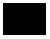 | 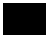 | 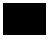 | 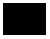 | 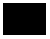 | 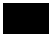 |
| 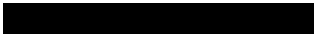 | 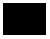 | 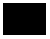 | 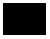 | 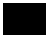 | 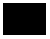 | 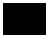 | 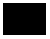 | 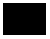 | 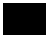 | 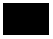 |
| 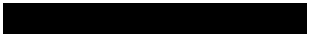 | 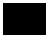 | 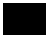 | 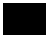 | 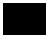 | 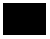 | 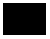 | 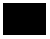 | 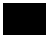 | 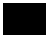 | 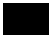 |
| 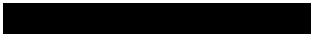 | 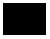 | 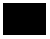 | 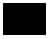 | 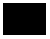 | 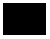 | 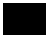 | 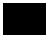 | 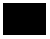 | 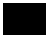 | 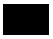 |
| VKDYVVVHGYFTEVY | 0.7 | 0.6 | 0.4 | 0.4 | 0.6 | 0.5 | 0.4 | 0.2 | 0.2 | 709 |
| KDYVVVHGYFTEVYY | 0.7 | 0.6 | 0.5 | 0.4 | 0.6 | 0.5 | 0.3 | 0.2 | 0.2 | 710 |
| DYVVVHGYFTEVYYQ | 0.6 | 0.5 | 0.4 | 0.4 | 0.6 | 0.4 | 0.3 | 0.3 | 0.2 | 711 |
| YVVVHGYFTEVYYQL | 0.7 | 0.6 | 0.5 | 0.3 | 0.5 | 0.4 | 0.2 | 0.1 | 0.2 | 712 |
| VVVHGYFTEVYYQLE | 0.8 | 0.6 | 0.6 | 0.4 | 0.6 | 0.4 | 0.3 | 0.3 | 0.2 | 713 |
| VVHGYFTEVYYQLES | 0.8 | 0.7 | 0.4 | 0.3 | 0.5 | 0.4 | 0.3 | 0.2 | 0.2 | 714 |
| VHGYFTEVYYQLEST | 0.7 | 0.5 | 0.4 | 0.4 | 0.4 | 0.3 | 0.3 | 0.1 | 0.2 | 715 |
| HGYFTEVYYQLESTQ | 0.6 | 0.4 | 0.4 | 0.3 | 0.4 | 0.4 | 0.0 | 0.2 | 0.2 | 716 |
| GYFTEVYYQLESTQI | 0.7 | 0.5 | 0.5 | 0.4 | 0.6 | 0.5 | 0.4 | 0.3 | 0.3 | 717 |
| YFTEVYYQLESTQIT | 0.7 | 0.6 | 0.4 | 0.4 | 0.6 | 0.6 | 0.3 | 0.3 | 0.3 | 718 |
| FTEVYYQLESTQITT | 0.8 | 0.7 | 0.5 | 0.5 | 0.7 | 0.7 | 0.5 | 0.5 | 0.3 | 719 |
| TEVYYQLESTQITTD | 0.8 | 0.8 | 0.5 | 0.5 | 0.9 | 0.6 | 0.6 | 0.5 | 0.4 | 720 |
| EVYYQLESTQITTDT | 0.7 | 0.6 | 0.4 | 0.5 | 0.7 | 0.4 | 0.3 | 0.2 | 0.3 | 721 |
| VYYQLESTQITTDTG | 0.6 | 0.5 | 0.4 | 0.4 | 0.6 | 0.5 | 0.3 | 0.2 | 0.2 | 722 |
| YYQLESTQITTDTGI | 0.7 | 0.7 | 0.4 | 0.5 | 0.7 | 0.5 | 0.3 | 0.2 | 0.2 | 723 |
| YQLESTQITTDTGIE | 0.6 | 0.5 | 0.4 | 0.5 | 0.6 | 0.5 | 0.5 | 0.4 | 0.2 | 724 |
| QLESTQITTDTGIEN | 0.6 | 0.5 | 0.4 | 0.4 | 0.5 | 0.5 | 0.4 | 0.2 | 0.3 | 725 |
| LESTQITTDTGIENA | 0.6 | 0.5 | 0.4 | 0.4 | 0.5 | 0.4 | 0.2 | 0.1 | 0.2 | 726 |
| ESTQITTDTGIENAT | 0.6 | 0.4 | 0.3 | 0.4 | 0.5 | 0.5 | 0.1 | 0.2 | 0.2 | 727 |
| STQITTDTGIENATF | 0.5 | 0.4 | 0.4 | 0.5 | 0.6 | 0.4 | 0.2 | 0.3 | 0.2 | 728 |
| TQITTDTGIENATFF | 0.7 | 0.6 | 0.5 | 0.6 | 0.7 | 0.5 | 0.5 | 0.7 | 0.3 | 729 |
| QITTDTGIENATFFI | 0.7 | 0.6 | 0.4 | 0.4 | 0.6 | 0.4 | 0.3 | 0.3 | 0.3 | 730 |
| ITTDTGIENATFFIF | 0.8 | 0.7 | 0.6 | 0.5 | 0.6 | 0.5 | 0.5 | 0.3 | 0.3 | 731 |
| TTDTGIENATEFIFN | 0.8 | 0.5 | 0.6 | 0.5 | 0.6 | 0.5 | 0.5 | 0.4 | 0.3 | 732 |
| TDTGIENATFFIFNK | 0.7 | 0.4 | 0.5 | 0.7 | 0.6 | 0.5 | 0.0 | 0.5 | 0.3 | 733 |
| DTGIENATFFIFNKL | 0.7 | 0.5 | 0.5 | 0.4 | 0.7 | 0.6 | 0.4 | 0.3 | 0.4 | 734 |
| TGIENATFFIFNKLV | 0.7 | 0.5 | 0.6 | 0.5 | 0.7 | 0.7 | 0.6 | 0.4 | 0.3 | 735 |
| GIENATFFIFNKLVK | 0.6 | 0.5 | 0.5 | 0.5 | 0.6 | 0.6 | 0.5 | 0.2 | 0.3 | 736 |
| IENATFFIFNKLVKD | 0.6 | 0.5 | 0.5 | 0.3 | 0.5 | 0.5 | 0.4 | 0.2 | 0.2 | 737 |
| ENATFFIFNKLVKDP | 0.6 | 0.4 | 0.5 | 0.4 | 0.5 | 0.4 | 0.2 | 0.0 | 0.2 | 738 |
| NATFFIFNKLVKDPP | 0.6 | 0.5 | 0.4 | 0.4 | 0.5 | 0.4 | 0.2 | 0.2 | 0.2 | 739 |
| 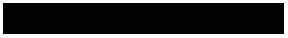 | 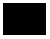 | 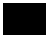 | 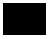 | 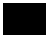 | 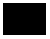 | 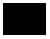 | 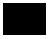 | 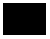 | 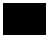 | 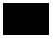 |
| 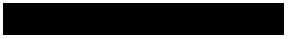 | 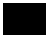 | 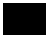 | 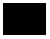 | 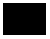 | 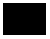 | 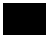 | 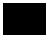 | 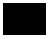 | 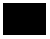 | 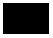 |
| 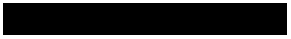 | 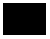 | 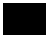 | 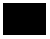 | 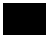 | 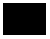 | 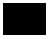 | 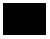 | 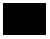 | 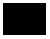 | 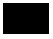 |
| 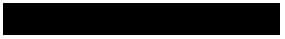 | 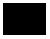 | 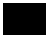 | 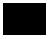 | 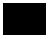 | 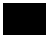 | 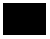 | 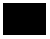 | 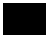 | 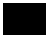 | 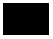 |
| 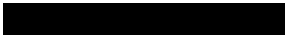 | 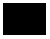 | 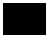 | 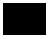 | 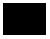 | 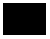 | 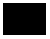 | 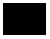 | 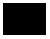 | 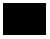 | 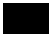 |
| 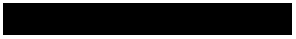 | 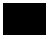 | 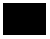 | 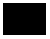 | 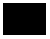 | 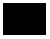 | 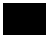 | 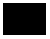 | 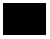 | 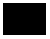 | 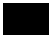 |
| 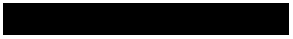 | 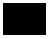 | 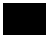 | 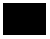 | 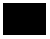 | 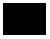 | 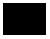 | 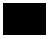 | 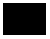 | 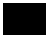 | 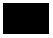 |
| 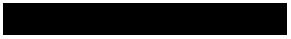 | 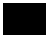 | 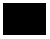 | 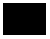 | 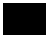 | 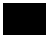 | 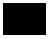 | 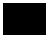 | 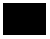 | 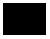 | 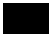 |
| 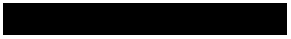 | 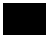 | 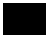 | 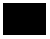 | 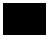 | 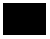 | 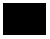 | 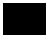 | 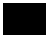 | 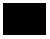 | 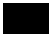 |
| 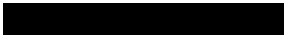 | 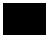 | 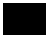 | 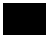 | 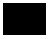 | 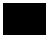 | 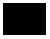 | 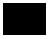 | 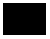 | 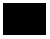 | 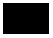 |
| 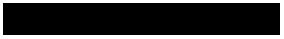 | 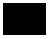 | 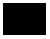 | 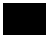 | 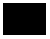 | 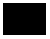 | 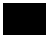 | 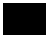 | 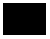 | 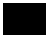 | 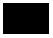 |
| DPPNVQIHTIDGSSG | 0.5 | 0.4 | 0.3 | 0.3 | 0.5 | 0.4 | 0.2 | 0.2 | 0.2 | 740 |
| PPNVQIHTIDGSSGV | 0.6 | 0.5 | 0.4 | 0.4 | 0.7 | 0.6 | 0.4 | 0.3 | 0.3 | 741 |
| PNVQIHTIDGSSGVA | 0.6 | 0.4 | 0.5 | 0.4 | 0.7 | 0.6 | 0.4 | 0.2 | 0.3 | 742 |
| NVQIHTIDGSSGVAN | 0.7 | 0.5 | 0.5 | 0.4 | 0.7 | 0.6 | 0.4 | 0.3 | 0.3 | 743 |
| VQIHTIDGSSGVANP | 0.7 | 0.5 | 0.6 | 0.6 | 0.7 | 0.6 | 0.4 | 0.2 | 0.3 | 744 |
| QIHTIDGSSGVANPA | 0.6 | 0.5 | 0.6 | 0.5 | 0.7 | 0.6 | 0.3 | 0.4 | 0.3 | 745 |
| IHTIDGSSGVANPAM | 0.7 | 0.7 | 0.8 | 0.5 | 0.7 | 0.8 | 0.6 | 0.5 | 0.5 | 746 |
| HTIDGSSGVANPAMD | 0.7 | 0.7 | 0.5 | 0.5 | 0.7 | 0.7 | 0.4 | 0.6 | 0.3 | 747 |
| TIDGSSGVANPAMDP | 0.6 | 0.6 | 0.6 | 0.6 | 0.6 | 0.6 | 0.3 | 0.4 | 0.4 | 748 |
| IDGSSGVANPAMDPI | 0.7 | 0.6 | 0.7 | 0.5 | 0.6 | 0.6 | 0.4 | 0.3 | 0.4 | 749 |
| 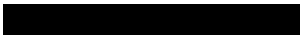 | 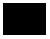 | 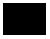 | 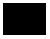 | 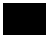 | 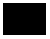 | 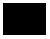 | 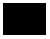 | 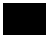 | 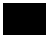 | 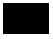 |
| 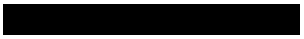 | 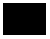 | 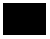 | 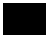 | 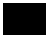 | 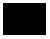 | 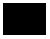 | 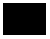 | 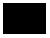 | 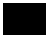 | 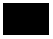 |
| 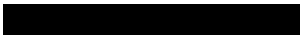 | 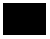 | 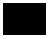 | 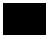 | 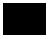 | 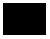 | 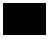 | 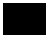 | 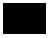 | 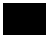 | 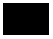 |
| 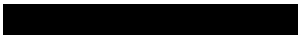 | 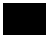 | 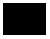 | 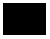 | 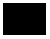 | 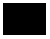 | 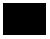 | 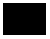 | 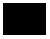 | 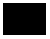 | 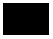 |
| 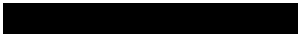 | 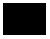 | 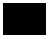 | 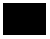 | 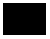 | 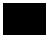 | 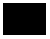 | 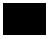 | 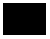 | 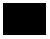 | 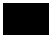 |
| 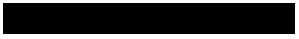 | 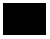 | 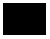 | 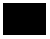 | 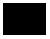 | 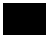 | 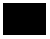 | 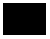 | 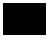 | 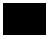 | 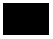 |
| 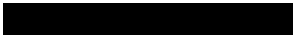 | 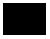 | 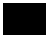 | 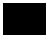 | 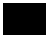 | 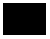 | 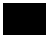 | 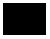 | 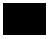 | 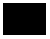 | 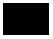 |
| 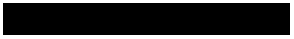 | 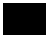 | 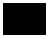 | 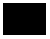 | 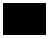 | 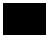 | 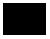 | 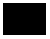 | 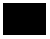 | 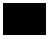 | 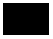 |
| 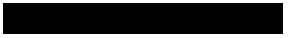 | 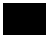 | 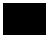 | 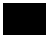 | 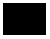 | 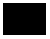 | 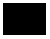 | 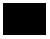 | 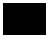 | 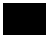 | 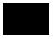 |
| 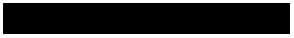 | 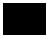 | 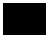 | 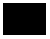 | 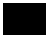 | 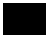 | 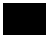 | 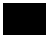 | 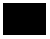 | 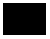 | 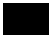 |
| 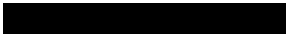 | 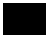 | 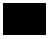 | 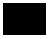 | 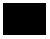 | 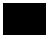 | 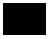 | 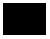 | 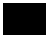 | 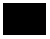 | 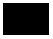 |
| 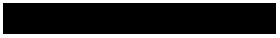 | 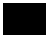 | 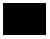 | 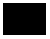 | 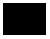 | 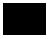 | 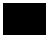 | 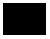 | 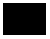 | 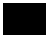 | 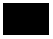 |

TABLE 2
Binding of the sera called SARS-yellow, SARS-green, 1a,
1b, 2, 6, 37, 62 and London to looped/cyclic peptides of
protein X1 of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MDLFMRFFTLGSITA | 0.5 | 0.3 | 0.4 | 1.0 | 0.4 | 0.5 | 0.5 | 0.4 | 0.8 | 607 |
| DLFMRFFTLGSITAQ | 0.5 | 0.3 | 0.3 | 0.3 | 0.5 | 0.5 | 0.2 | 0.2 | 0.2 | 608 |
| LFMRFFTLGSITAQP | 0.5 | 0.3 | 0.4 | 0.3 | 0.5 | 0.5 | 0.4 | 0.3 | 0.2 | 609 |
| FMRFFTLGSITAQPV | 0.5 | 0.3 | 0.4 | 0.4 | 0.5 | 0.5 | 0.3 | 0.2 | 0.2 | 610 |
| MRFFTLGSITAQPVK | 0.3 | 0.1 | 0.1 | 0.2 | 0.2 | 0.3 | 0.1 | 0.1 | 0.2 | 611 |
| 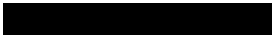 | 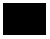 | 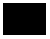 | 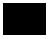 | 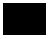 | 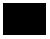 | 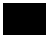 | 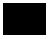 | 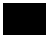 | 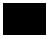 | 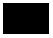 |
| 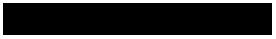 | 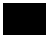 | 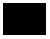 | 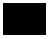 | 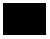 | 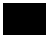 | 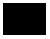 | 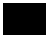 | 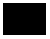 | 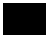 | 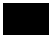 |
| 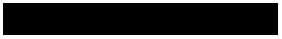 | 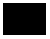 | 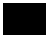 | 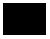 | 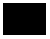 | 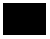 | 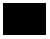 | 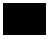 | 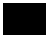 | 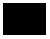 | 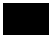 |
| 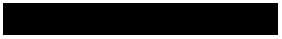 | 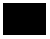 | 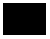 | 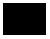 | 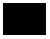 | 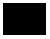 | 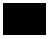 | 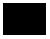 | 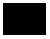 | 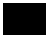 | 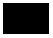 |
| 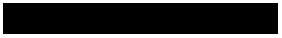 | 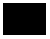 | 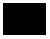 | 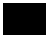 | 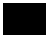 | 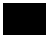 | 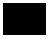 | 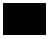 | 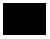 | 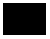 | 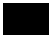 |
| 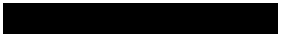 | 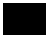 | 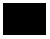 | 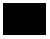 | 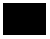 | 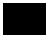 | 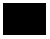 | 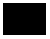 | 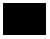 | 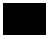 | 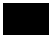 |
| 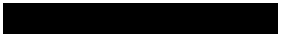 | 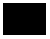 | 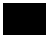 | 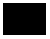 | 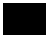 | 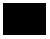 | 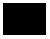 | 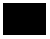 | 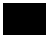 | 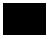 | 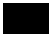 |
| 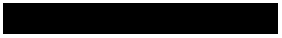 | 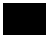 | 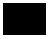 | 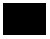 | 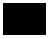 | 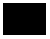 | 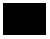 | 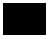 | 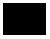 | 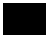 | 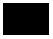 |
| 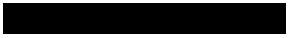 | 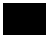 | 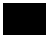 | 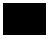 | 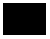 | 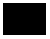 | 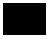 | 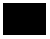 | 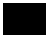 | 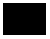 | 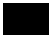 |
| 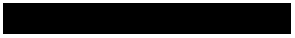 | 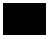 | 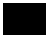 | 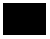 | 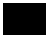 | 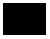 | 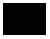 | 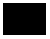 | 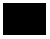 | 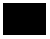 | 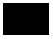 |
| 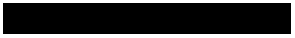 | 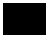 | 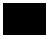 | 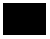 | 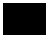 | 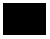 | 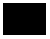 | 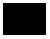 | 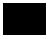 | 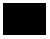 | 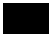 |
| 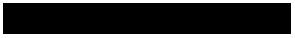 | 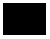 | 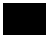 | 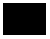 | 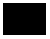 | 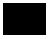 | 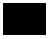 | 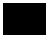 | 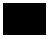 | 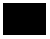 | 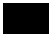 |
| 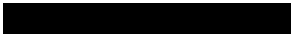 | 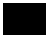 | 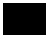 | 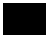 | 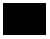 | 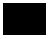 | 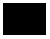 | 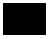 | 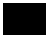 | 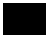 | 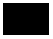 |
| 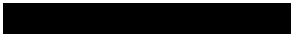 | 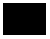 | 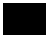 | 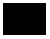 | 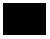 | 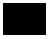 | 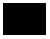 | 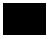 | 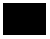 | 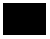 | 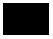 |
| 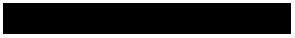 | 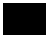 | 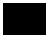 | 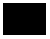 | 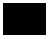 | 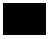 | 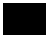 | 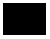 | 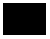 | 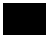 | 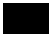 |
| 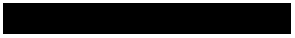 | 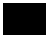 | 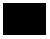 | 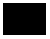 | 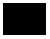 | 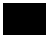 | 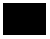 | 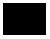 | 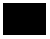 | 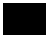 | 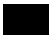 |
| 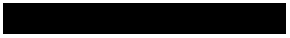 | 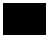 | 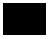 | 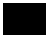 | 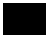 | 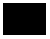 | 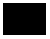 | 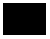 | 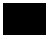 | 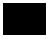 | 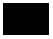 |
| 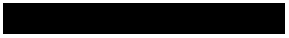 | 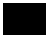 | 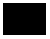 | 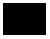 | 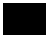 | 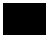 | 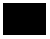 | 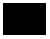 | 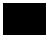 | 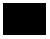 | 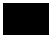 |
| 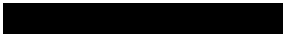 | 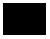 | 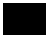 | 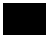 | 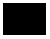 | 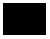 | 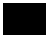 | 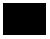 | 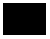 | 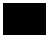 | 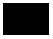 |
| 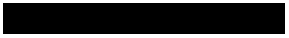 | 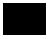 | 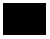 | 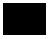 | 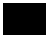 | 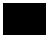 | 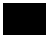 | 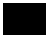 | 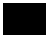 | 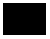 | 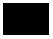 |
| 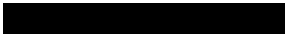 | 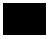 | 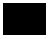 | 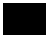 | 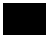 | 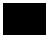 | 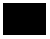 | 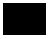 | 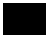 | 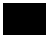 | 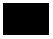 |
| 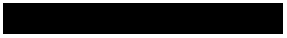 | 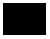 | 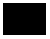 | 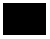 | 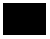 | 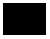 | 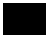 | 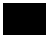 | 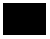 | 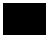 | 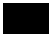 |
| 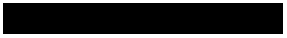 | 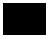 | 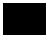 | 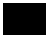 | 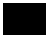 | 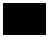 | 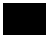 | 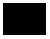 | 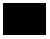 | 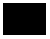 | 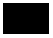 |
| 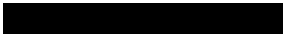 | 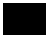 | 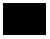 | 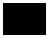 | 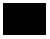 | 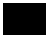 | 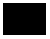 | 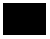 | 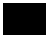 | 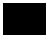 | 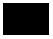 |
| HATATIPLQASLPFG | 0.8 | 0.6 | 0.5 | 0.5 | 0.6 | 0.6 | 0.4 | 0.4 | 0.2 | 612 |
| ATATIPLQASLPFGW | 0.6 | 0.5 | 0.5 | 0.5 | 0.7 | 0.7 | 0.4 | 0.3 | 0.3 | 613 |
| TATIPLQASLPFGWL | 0.7 | 0.6 | 0.5 | 0.5 | 0.8 | 0.8 | 0.5 | 0.5 | 0.3 | 614 |
| ATIPLQASLPFGWLV | 0.6 | 0.5 | 0.6 | 0.4 | 0.7 | 0.7 | 0.3 | 0.4 | 0.3 | 615 |
| TIPLQASLPFGWLVI | 0.6 | 0.4 | 0.5 | 0.4 | 0.6 | 0.7 | 0.5 | 0.4 | 0.2 | 616 |
| IPLQASLPFGWLVIG | 0.6 | 0.4 | 0.4 | 0.3 | 0.5 | 0.6 | 0.3 | 0.3 | 0.2 | 617 |
| PLQASLPFGWLVIGV | 0.5 | 0.3 | 0.4 | 0.4 | 0.5 | 0.7 | 0.4 | 0.3 | 0.2 | 618 |
| LQASLPFGWLVIGVA | 0.4 | 0.3 | 0.3 | 0.3 | 0.4 | 0.5 | 0.4 | 0.2 | 0.2 | 619 |
| QASLPFGWLVIGVAF | 0.4 | 0.3 | 0.2 | 0.3 | 0.4 | 0.4 | 0.2 | 0.2 | 0.2 | 620 |
| ASLPFGWLVIGVAFL | 0.5 | 0.3 | 0.4 | 0.2 | 0.3 | 0.4 | 0.3 | 0.2 | 0.2 | 621 |
| SLPFGWLVIGVAFLA | 0.3 | 0.2 | 0.2 | 0.0 | 0.3 | 0.3 | 0.2 | 0.3 | 0.2 | 622 |
| LPFGWLVIGVAFLAV | 0.5 | 0.3 | 0.4 | 0.3 | 0.6 | 0.6 | 0.3 | 0.3 | 0.2 | 623 |
| PFGWLVIGVAFLAVF | 0.5 | 0.4 | 0.5 | 0.2 | 0.5 | 0.5 | 0.5 | 0.4 | 0.2 | 624 |
| FGWLVIGVAFLAVFQ | 0.7 | 0.8 | 0.7 | 0.5 | 0.6 | 0.7 | 0.6 | 0.4 | 0.3 | 625 |
| GWLVIGVAFLAVFQS | 0.7 | 0.5 | 0.7 | 0.4 | 0.6 | 0.8 | 0.6 | 0.4 | 0.3 | 626 |
| WLVIGVAFLAVFQSA | 0.5 | 0.3 | 0.4 | 0.2 | 0.5 | 0.5 | 0.5 | 0.2 | 0.2 | 627 |
| LVIGVAFLAVFQSAT | 0.8 | 0.6 | 0.7 | 0.7 | 0.8 | 1.0 | 0.7 | 0.5 | 0.3 | 628 |
| VIGVAFLAVFQSATK | 0.4 | 0.3 | 0.3 | 0.6 | 0.4 | 0.4 | 0.5 | 0.2 | 0.7 | 629 |
| IGVAFLAVFQSATKI | 0.5 | 0.4 | 0.4 | 0.3 | 0.5 | 0.6 | 0.3 | 0.2 | 0.2 | 630 |
| GVAFLAVFQSATKII | 0.6 | 0.4 | 0.6 | 0.5 | 0.6 | 0.6 | 0.3 | 0.3 | 0.4 | 631 |
| VAFLAVFQSATKIIA | 0.6 | 0.4 | 0.6 | 0.7 | 0.6 | 0.6 | 0.5 | 0.4 | 0.7 | 632 |
| AFLAVFQSATKIIAL | 0.5 | 0.4 | 0.5 | 1.3 | 0.6 | 0.6 | 0.6 | 0.3 | 1.6 | 633 |
| FLAVFQSATKIIALN | 0.6 | 0.4 | 0.5 | 1.0 | 0.5 | 0.6 | 0.4 | 0.3 | 1.4 | 634 |
| LAVFQSATKIIALNK | 0.5 | 0.4 | 0.5 | 0.6 | 0.5 | 0.6 | 0.5 | 0.3 | 0.7 | 635 |
| AVFQSATKIIALNKR | 0.4 | 0.4 | 0.6 | 0.7 | 0.6 | 0.6 | 0.4 | 0.2 | 0.9 | 492 |
| VFQSATKIIALNKRW | 0.5 | 0.4 | 0.4 | 0.6 | 0.4 | 0.6 | 0.2 | 0.2 | 0.3 | 493 |
| FQSATKIIALNKRWQ | 0.5 | 0.3 | 0.4 | 0.5 | 0.5 | 0.5 | 0.2 | 0.3 | 0.2 | 494 |
| QSATKIIALNKRWQL | 0.4 | 0.4 | 0.5 | 0.6 | 0.6 | 0.6 | 0.4 | 0.4 | 0.4 | 495 |
| SATKIIALNKRWQLA | 1.2 | 0.8 | 1.2 | 0.9 | 1.0 | 1.5 | 0.9 | 1.0 | 0.5 | 496 |
| ATKIIALNKRWQLAL | 0.6 | 0.4 | 0.5 | 0.5 | 0.6 | 0.7 | 0.6 | 0.3 | 0.3 | 497 |
| TKIIALNKRWQLALY | 0.6 | 0.4 | 0.5 | 0.5 | 0.6 | 0.6 | 0.5 | 0.3 | 0.3 | 498 |
| KIIALNKRWQLALYK | 0.7 | 0.5 | 0.7 | 0.8 | 0.8 | 0.7 | 0.5 | 0.3 | 0.3 | 499 |
| IIALNKRWQLALYKG | 0.6 | 0.4 | 0.5 | 0.6 | 0.6 | 0.6 | 0.5 | 0.2 | 0.4 | 500 |
| IALNKRWQLALYKGF | 0.5 | 0.4 | 0.5 | 0.4 | 0.5 | 0.7 | 0.6 | 0.4 | 0.3 | 501 |
| ALNKRWQLALYKGFQ | 0.8 | 0.6 | 0.7 | 0.6 | 0.6 | 0.7 | 0.6 | 0.4 | 0.3 | 502 |
| LNKRWQLALYKGFQF | 0.6 | 0.4 | 0.5 | 0.5 | 0.5 | 0.6 | 0.4 | 0.1 | 0.3 | 503 |
| NKRWQLALYKGFQFI | 0.6 | 0.4 | 0.5 | 0.5 | 0.6 | 0.7 | 0.5 | 0.2 | 0.4 | 504 |
| KRWQLALYKGFQFIC | 0.6 | 0.4 | 0.5 | 0.8 | 0.6 | 0.6 | 0.5 | 0.3 | 1.1 | 636 |
| RWQLALYKGFQFICN | 0.6 | 0.4 | 0.5 | 0.9 | 0.6 | 0.6 | 0.6 | 0.3 | 1.4 | 637 |
| WQLALYKGFQFICNL | 0.6 | 0.3 | 0.4 | 1.3 | 0.5 | 0.5 | 0.5 | 0.3 | 1.5 | 638 |
| QLALYKGFQFICNLL | 0.5 | 0.4 | 0.4 | 1.2 | 0.5 | 0.5 | 0.5 | 0.3 | 1.5 | 639 |
| LALYKGFQFICNLLL | 0.5 | 0.3 | 0.4 | 1.4 | 0.5 | 0.5 | 0.5 | 0.2 | 1.4 | 640 |
| ALYKGFQFICNLLLL | 0.4 | 0.3 | 0.3 | 1.3 | 0.4 | 0.4 | 0.4 | 0.2 | 1.4 | 641 |
| LYKGFQFICNLLLLF | 0.5 | 0.3 | 0.3 | 1.5 | 0.4 | 0.5 | 0.3 | 0.2 | 1.7 | 642 |
| YKGFQFICNLLLLFV | 0.3 | 0.0 | 0.0 | 0.3 | 0.2 | 0.3 | 0.0 | 0.3 | 0.2 | 643 |
| KGFQFICNLLLLFVT | 0.6 | 0.4 | 0.4 | 1.0 | 0.6 | 0.6 | 0.6 | 0.4 | 1.1 | 644 |
| GFQFICNLLLLFVTI | 0.5 | 0.3 | 0.3 | 0.3 | 0.5 | 0.5 | 0.4 | 0.2 | 0.2 | 645 |
| FQFICNLLLLFVTIY | 0.2 | 0.0 | 0.0 | 0.2 | 0.0 | 0.2 | 0.0 | 0.4 | 0.1 | 646 |
| QFICNLLLLFVTIYS | 0.5 | 0.4 | 0.6 | 0.3 | 0.5 | 0.5 | 0.5 | 0.2 | 0.2 | 647 |
| FICNLLLLFVTIYSH | 0.5 | 0.3 | 0.4 | 0.2 | 0.5 | 0.5 | 0.5 | 0.3 | 0.2 | 648 |
| ICNLLLLFVTIYSHL | 0.4 | 0.3 | 0.4 | 0.2 | 0.4 | 0.5 | 0.4 | 0.2 | 0.2 | 649 |
| CNLLLLFVTIYSHLL | 0.5 | 0.4 | 0.4 | 0.3 | 0.5 | 0.5 | 0.4 | 0.2 | 0.2 | 650 |
| NLLLLFVTIYSHLLL | 0.5 | 0.4 | 0.4 | 0.2 | 0.4 | 0.5 | 0.4 | 0.2 | 0.2 | 651 |
| LLLLFVTIYSHLLLV | 0.5 | 0.3 | 0.5 | 0.3 | 0.6 | 0.6 | 0.5 | 0.2 | 0.2 | 652 |
| LLLFVTIYSHLLLVA | 0.5 | 0.3 | 0.4 | 0.2 | 0.5 | 0.4 | 0.4 | 0.2 | 0.2 | 653 |
| LLFVTIYSHLLLVAA | 0.4 | 0.3 | 0.3 | 0.2 | 0.4 | 0.5 | 0.4 | 0.2 | 0.2 | 654 |
| LFVTIYSHLLLVAAG | 0.5 | 0.3 | 0.4 | 0.2 | 0.5 | 0.5 | 0.4 | 0.3 | 0.2 | 655 |
| FVTIYSHLLLVAAGM | 0.5 | 0.3 | 0.3 | 0.3 | 0.5 | 0.5 | 0.3 | 0.2 | 0.2 | 656 |
| VTIYSHLLLVAAGME | 0.5 | 0.4 | 0.5 | 0.3 | 0.5 | 0.4 | 0.3 | 0.3 | 0.2 | 657 |
| TIYSHLLLVAAGMEA | 0.5 | 0.4 | 0.3 | 0.3 | 0.5 | 0.5 | 0.3 | 0.2 | 0.2 | 658 |
| IYSHLLLVAAGMEAQ | 0.5 | 0.4 | 0.3 | 0.3 | 0.5 | 0.5 | 0.2 | 0.2 | 0.2 | 659 |
| YSHLLLVAAGMEAQF | 0.4 | 0.4 | 0.5 | 0.3 | 0.7 | 0.5 | 0.3 | 0.5 | 0.3 | 660 |
| SHLLLVAAGMEAQFL | 0.2 | 0.5 | 0.1 | 0.1 | 0.1 | 0.2 | 0.0 | 0.5 | 0.1 | 661 |
| HLLLVAAGMEAQFLY | 0.5 | 0.6 | 0.6 | 0.1 | 0.6 | 0.6 | 0.3 | 0.4 | 0.2 | 662 |
| LLLVAAGMEAQFLYL | 0.6 | 0.5 | 0.6 | 0.3 | 0.7 | 0.7 | 0.6 | 0.5 | 0.2 | 663 |
| LLVAAGMEAQFLYLY | 0.6 | 0.5 | 0.6 | 0.3 | 0.6 | 0.6 | 0.5 | 0.4 | 0.2 | 664 |
| LVAAGMEAQFLYLYA | 0.5 | 0.4 | 0.5 | 0.3 | 0.5 | 0.5 | 0.6 | 0.3 | 0.2 | 665 |
| VAAGMEAQFLYLYAL | 0.5 | 0.4 | 0.4 | 0.3 | 0.5 | 0.5 | 0.5 | 0.3 | 0.2 | 666 |
| AAGMEAQFLYLYALI | 0.6 | 0.4 | 0.5 | 0.3 | 0.6 | 0.6 | 0.4 | 0.3 | 0.2 | 667 |
| AGMEAQFLYLYALIY | 0.5 | 0.4 | 0.4 | 0.3 | 0.5 | 0.6 | 0.3 | 0.2 | 0.2 | 668 |
| GMEAQFLYLYALIYF | 0.6 | 0.4 | 0.5 | 0.3 | 0.4 | 0.5 | 0.4 | 0.3 | 0.2 | 669 |
| MEAQFLYLYALIYFL | 0.5 | 0.3 | 0.4 | 0.2 | 0.8 | 0.5 | 0.4 | 0.2 | 0.2 | 670 |
| EAQFLYLYALIYFLQ | 0.5 | 0.3 | 0.4 | 0.2 | 0.5 | 0.5 | 0.3 | 0.2 | 0.2 | 671 |
| AQFLYLYALIYFLQC | 0.5 | 0.3 | 0.4 | 0.2 | 0.4 | 0.5 | 0.3 | 0.3 | 0.2 | 672 |
| QFLYLYALTYFLQCI | 0.5 | 0.3 | 0.3 | 0.2 | 0.5 | 0.5 | 0.3 | 0.2 | 0.2 | 673 |
| FLYLYALIYFLQCIN | 0.4 | 0.3 | 0.3 | 0.2 | 0.4 | 0.4 | 0.3 | 0.2 | 0.2 | 674 |
| LYLYALIYFLQCINA | 0.3 | 0.3 | 0.2 | 0.3 | 0.4 | 0.4 | 0.3 | 0.2 | 0.2 | 675 |
| YLYALIYFLQCINAC | 0.4 | 0.2 | 0.3 | 0.2 | 0.4 | 0.4 | 0.2 | 0.1 | 0.2 | 676 |
| LYALIYFLQCINACR | 0.4 | 0.3 | 0.6 | 0.7 | 0.6 | 0.5 | 0.2 | 0.2 | 1.2 | 677 |
| YALIYFLQCINACRI | 0.2 | 0.1 | 0.1 | 0.0 | 0.1 | 0.2 | 0.0 | 0.2 | 0.1 | 678 |
| ALIYFLQCINACRII | 0.3 | 0.2 | 0.4 | 0.9 | 0.3 | 0.3 | 0.1 | 0.3 | 1.3 | 679 |
| LIYFLQCINACRIIM | 0.2 | 0.4 | 0.7 | 1.2 | 0.2 | 0.4 | 0.6 | 0.6 | 1.1 | 680 |
| IYFLQCINACRIIMR | 0.6 | 0.4 | 0.8 | 1.0 | 0.6 | 0.7 | 0.6 | 0.2 | 1.6 | 681 |
| YFLQCINACRIIMRC | 0.6 | 0.4 | 0.4 | 1.6 | 0.5 | 0.6 | 0.6 | 0.2 | 1.6 | 682 |
| FLQCINACRIIMRCW | 0.6 | 0.4 | 0.5 | 0.7 | 0.6 | 0.6 | 0.6 | 0.2 | 1.0 | 683 |
| LQCINACRIIMRCWL | 0.5 | 0.3 | 0.6 | 1.4 | 0.6 | 0.9 | 0.4 | 0.2 | 1.7 | 505 |
| QCINACRIIMRCWLC | 0.6 | 0.4 | 0.6 | 0.8 | 0.6 | 0.7 | 0.4 | 0.2 | 1.4 | 506 |
| CINACRIIMRCWLCW | 0.7 | 0.4 | 0.6 | 0.7 | 0.6 | 0.7 | 0.5 | 0.3 | 0.8 | 507 |
| INACRIIMRCWLCWK | 0.6 | 0.4 | 0.6 | 0.6 | 0.5 | 0.6 | 0.5 | 0.3 | 0.4 | 33 |
| NACRIIMRCWLCWKC | 0.6 | 0.5 | 0.5 | 0.6 | 0.5 | 0.7 | 0.4 | 0.3 | 0.3 | 34 |
| ACRTIMRCWLCWKCK | 0.7 | 0.9 | 0.6 | 0.3 | 0.8 | 0.5 | 0.7 | 1.2 | 0.2 | 35 |
| CRIIMRCWLCWKCKS | 0.5 | 0.4 | 0.5 | 0.5 | 0.6 | 0.6 | 0.4 | 0.2 | 0.4 | 36 |
| RIIMRCWLCWKCKSK | 0.2 | 0.2 | 0.1 | 0.1 | 0.2 | 0.3 | 0.1 | 0.1 | 0.1 | 37 |
| IIMRCWLCWKCKSKN | 0.5 | 0.3 | 0.3 | 0.5 | 0.4 | 0.5 | 0.3 | 0.2 | 0.2 | 38 |
| IMRCWLCWKCKSKNP | 0.3 | 0.1 | 0.1 | 0.2 | 0.3 | 0.3 | 0.0 | 0.1 | 0.1 | 39 |
| MRCWLCWKCKSKNPL | 0.2 | 0.2 | 0.0 | 0.1 | 0.2 | 0.3 | 0.2 | 0.1 | 0.2 | 40 |
| RCWLCWKCKSKNPLL | 0.7 | 0.5 | 0.7 | 0.8 | 0.8 | 0.7 | 0.7 | 0.4 | 0.4 | 41 |
| CWLCWKCKSKNPLLY | 0.7 | 0.4 | 0.5 | 0.7 | 0.7 | 0.7 | 0.5 | 0.3 | 0.3 | 42 |
| WLCWKCKSKNPLLYD | 0.8 | 0.6 | 0.6 | 0.6 | 0.9 | 0.7 | 0.6 | 0.4 | 0.3 | 43 |
| LCWKCKSKNPLLYDA | 0.8 | 0.5 | 0.7 | 0.8 | 0.7 | 0.8 | 0.7 | 0.3 | 0.3 | 44 |
| CWKCKSKNPLLYDAN | 0.7 | 0.6 | 0.5 | 0.4 | 0.7 | 0.6 | 0.6 | 0.2 | 0.3 | 45 |
| WKCKSKNPLLYDANY | 0.6 | 0.5 | 0.4 | 0.3 | 0.6 | 0.6 | 0.5 | 0.3 | 0.2 | 684 |
| KCKSKNPLLYDANYF | 0.7 | 0.5 | 0.4 | 0.4 | 0.8 | 0.7 | 0.5 | 0.3 | 0.3 | 685 |
| CKSKNPLLYDANYFV | 0.8 | 0.6 | 0.5 | 0.4 | 0.8 | 0.8 | 0.5 | 0.3 | 0.2 | 686 |
| KSKNPLLYDANYFVC | 0.6 | 0.5 | 0.4 | 0.3 | 0.6 | 0.6 | 0.4 | 0.4 | 0.2 | 687 |
| SKNPLLYDANYFVCW | 0.6 | 0.5 | 0.5 | 0.4 | 0.6 | 0.6 | 0.5 | 0.4 | 0.2 | 688 |
| KNPLLYDANYFVCWH | 0.6 | 0.5 | 0.4 | 0.4 | 0.6 | 0.6 | 0.4 | 0.3 | 0.2 | 689 |
| NPLLYDANYFVCWHT | 0.5 | 0.4 | 0.4 | 0.3 | 0.5 | 0.6 | 0.4 | 0.3 | 0.2 | 690 |
| PLLYDANYFVCWHTH | 0.6 | 0.6 | 0.4 | 0.4 | 0.7 | 0.7 | 0.4 | 0.3 | 0.2 | 691 |
| LLYDANYFVCWHTHN | 0.6 | 0.5 | 0.4 | 0.4 | 0.6 | 0.6 | 0.4 | 0.3 | 0.2 | 692 |
| LYDANYFVCWHTHNY | 0.6 | 0.5 | 0.4 | 0.4 | 0.6 | 0.6 | 0.3 | 0.3 | 0.2 | 693 |
| 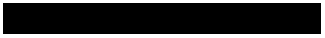 | 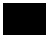 | 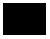 | 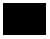 | 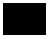 | 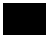 | 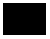 | 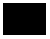 | 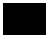 | 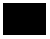 | 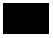 |
| 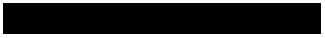 | 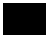 | 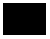 | 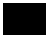 | 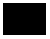 | 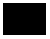 | 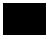 | 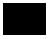 | 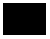 | 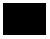 | 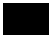 |
| 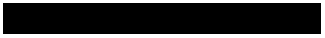 | 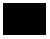 | 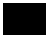 | 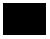 | 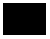 | 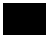 | 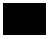 | 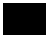 | 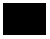 | 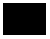 | 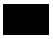 |
| 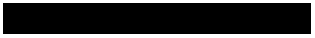 | 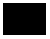 | 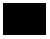 | 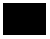 | 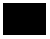 | 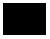 | 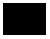 | 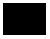 | 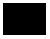 | 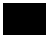 | 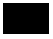 |
| 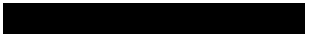 | 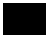 | 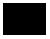 | 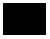 | 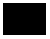 | 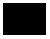 | 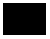 | 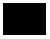 | 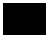 | 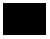 | 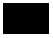 |
| 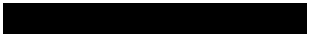 | 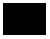 | 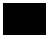 | 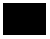 | 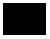 | 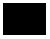 | 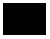 | 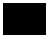 | 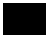 | 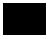 | 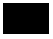 |
| 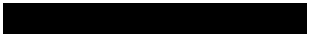 | 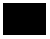 | 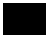 | 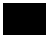 | 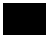 | 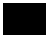 | 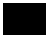 | 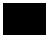 | 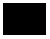 | 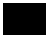 | 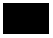 |
| 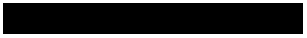 | 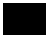 | 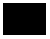 | 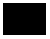 | 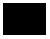 | 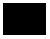 | 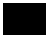 | 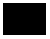 | 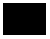 | 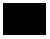 | 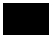 |
| 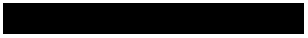 | 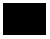 | 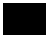 | 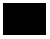 | 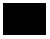 | 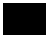 | 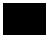 | 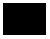 | 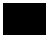 | 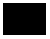 | 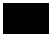 |
| 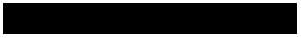 | 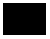 | 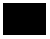 | 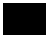 | 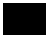 | 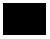 | 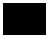 | 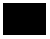 | 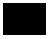 | 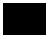 | 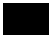 |
| 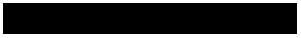 | 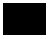 | 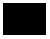 | 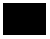 | 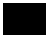 | 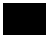 | 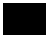 | 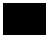 | 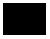 | 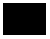 | 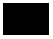 |
| 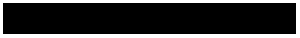 | 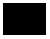 | 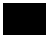 | 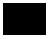 | 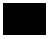 | 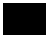 | 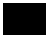 | 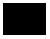 | 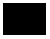 | 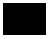 | 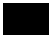 |
| 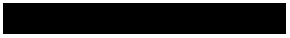 | 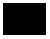 | 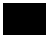 | 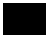 | 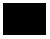 | 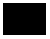 | 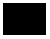 | 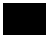 | 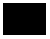 | 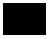 | 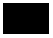 |
| 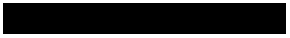 | 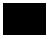 | 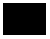 | 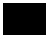 | 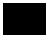 | 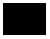 | 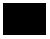 | 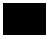 | 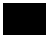 | 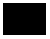 | 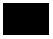 |
| 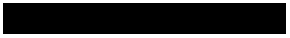 | 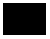 | 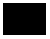 | 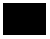 | 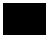 | 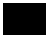 | 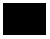 | 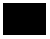 | 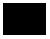 | 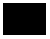 | 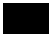 |
| 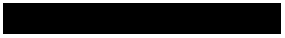 | 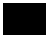 | 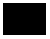 | 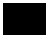 | 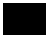 | 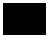 | 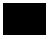 | 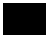 | 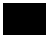 | 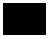 | 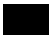 |
| CIPYNSVTDTIVVTE | 0 4 | 0.4 | 0.5 | 0.2 | 0.6 | 0.5 | 0.4 | 0.3 | 0.2 | 694 |
| IPYNSVTDTIVVTEG | 0.5 | 0.4 | 0.4 | 0.3 | 0.5 | 0.5 | 0.2 | 0.3 | 0.2 | 695 |
| PYNSVTDTIVVTEGD | 0.4 | 0.3 | 0.3 | 0.1 | 0.3 | 0.3 | 0.4 | 0.5 | 0.2 | 696 |
| YNSVTDTIVVTEGDG | 0.4 | 0.3 | 0.2 | 0.2 | 0.4 | 0.3 | 0.3 | 0.3 | 0.2 | 697 |
| NSVTDTIVVTEGDGI | 0.4 | 0.3 | 0.2 | 0.2 | 0.4 | 0.4 | 0.3 | 0.3 | 0.2 | 698 |
| SVTDTIVVTEGDGIS | 0.3 | 0.2 | 0.1 | 0.1 | 0.3 | 0.3 | 0.2 | 0.2 | 0.1 | 699 |
| VTDTIVVTEGDGIST | 0.4 | 0.3 | 0.2 | 0.1 | 0.4 | 0.4 | 0.5 | 0.2 | 0.2 | 700 |
| TDTIVVTEGDGISTP | 0.4 | 0.3 | 0.2 | 0.2 | 0.3 | 0.3 | 0.4 | 0.2 | 0.1 | 701 |
| DTIVVTEGDGISTPK | 0.3 | 0.2 | 0.1 | 0.2 | 0.3 | 0.3 | 0.1 | 0.1 | 0.2 | 702 |
| TIVVTEGDGISTPKL | 0.7 | 0.7 | 0.5 | 0.4 | 0.7 | 0.6 | 0.5 | 0.6 | 0.2 | 703 |
| IVVTEGDGISTPKLK | 0.3 | 0.2 | 0.1 | 0.1 | 0.2 | 0.3 | 0.1 | 0.0 | 0.1 | 704 |
| VVTEGDGISTPKLKE | 0.3 | 0.3 | 0.1 | 0.2 | 0.3 | 0.3 | 0.2 | 0.1 | 0.2 | 705 |
| VTEGDGISTPKLKED | 0.3 | 0.2 | 0.1 | 0.1 | 0.3 | 0.3 | 0.1 | 0.1 | 0.2 | 706 |
| TEGDGISTPKLKEDY | 0.5 | 0.4 | 0.5 | 0.4 | 0.4 | 0.4 | 0.3 | 0.3 | 0.2 | 707 |
| EGDGISTPKLKEDYQ | 0.4 | 0.3 | 0.2 | 0.2 | 0.3 | 0.3 | 0.1 | 0.2 | 0.2 | 708 |
| 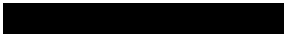 | 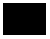 | 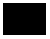 | 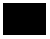 | 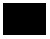 | 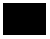 | 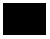 | 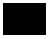 | 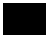 | 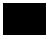 | 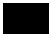 |
| 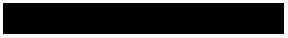 | 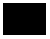 | 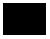 | 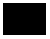 | 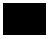 | 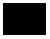 | 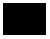 | 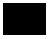 | 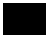 | 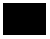 | 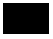 |
| 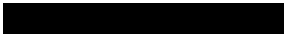 | 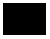 | 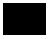 | 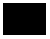 | 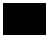 | 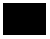 | 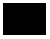 | 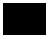 | 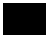 | 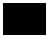 | 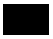 |
| 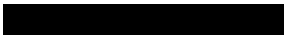 | 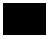 | 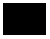 | 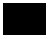 | 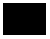 | 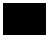 | 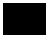 | 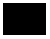 | 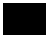 | 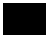 | 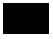 |
| 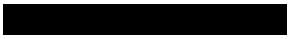 | 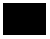 | 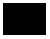 | 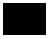 | 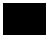 | 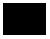 | 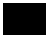 | 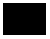 | 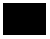 | 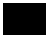 | 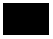 |
| 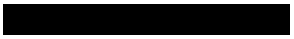 | 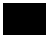 | 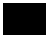 | 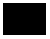 | 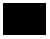 | 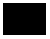 | 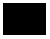 | 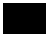 | 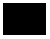 | 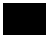 | 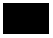 |
| 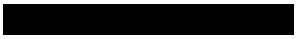 | 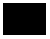 | 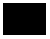 | 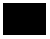 | 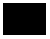 | 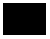 | 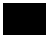 | 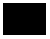 | 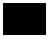 | 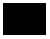 | 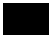 |
| 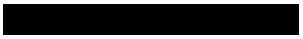 | 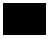 | 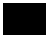 | 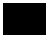 | 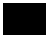 | 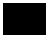 | 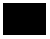 | 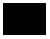 | 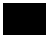 | 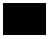 | 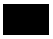 |
| 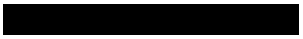 | 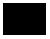 | 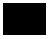 | 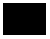 | 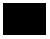 | 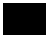 | 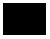 | 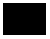 | 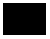 | 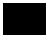 | 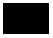 |
| 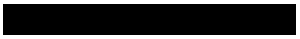 | 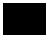 | 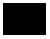 | 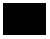 | 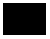 | 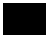 | 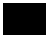 | 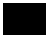 | 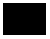 | 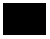 | 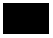 |
| 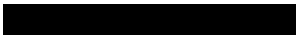 | 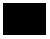 | 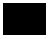 | 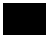 | 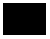 | 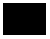 | 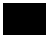 | 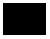 | 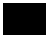 | 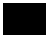 | 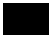 |
| 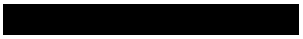 | 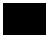 | 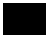 | 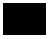 | 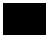 | 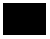 | 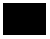 | 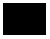 | 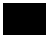 | 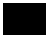 | 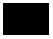 |
| 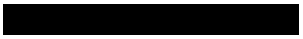 | 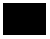 | 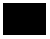 | 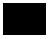 | 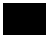 | 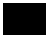 | 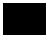 | 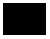 | 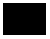 | 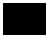 | 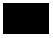 |
| 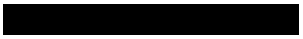 | 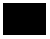 | 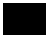 | 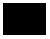 | 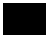 | 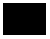 | 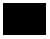 | 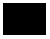 | 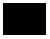 | 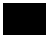 | 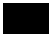 |
| 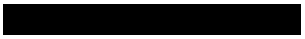 | 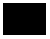 | 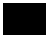 | 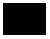 | 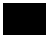 | 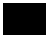 | 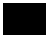 | 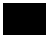 | 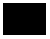 | 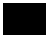 | 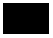 |
| 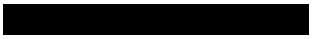 | 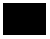 | 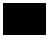 | 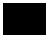 | 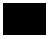 | 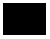 | 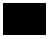 | 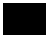 | 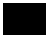 | 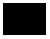 | 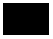 |
| 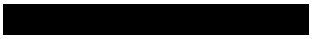 | 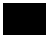 | 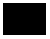 | 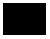 | 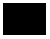 | 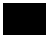 | 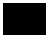 | 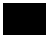 | 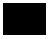 | 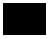 | 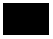 |
| 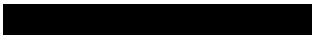 | 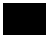 | 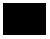 | 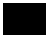 | 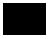 | 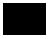 | 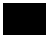 | 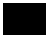 | 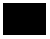 | 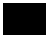 | 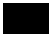 |
| 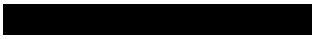 | 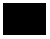 | 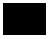 | 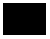 | 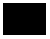 | 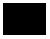 | 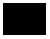 | 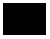 | 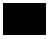 | 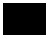 | 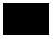 |
| 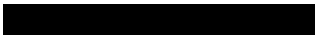 | 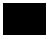 | 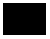 | 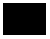 | 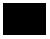 | 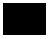 | 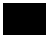 | 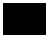 | 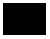 | 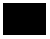 | 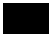 |
| 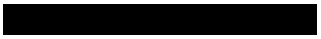 | 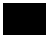 | 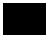 | 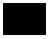 | 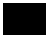 | 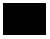 | 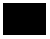 | 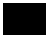 | 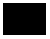 | 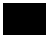 | 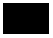 |
| 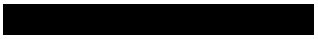 | 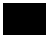 | 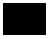 | 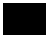 | 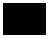 | 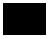 | 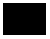 | 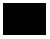 | 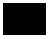 | 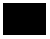 | 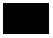 |
| 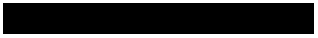 | 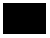 | 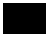 | 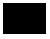 | 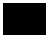 | 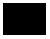 | 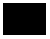 | 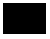 | 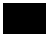 | 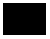 | 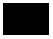 |
| 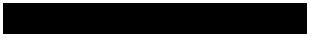 | 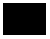 | 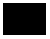 | 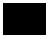 | 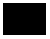 | 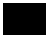 | 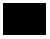 | 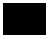 | 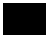 | 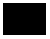 | 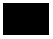 |
| 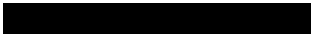 | 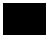 | 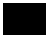 | 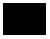 | 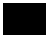 | 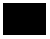 | 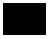 | 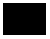 | 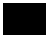 | 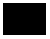 | 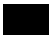 |
| VKDYVVVHGYFTEVY | 0.5 | 0.4 | 0.4 | 0.2 | 0.6 | 0.5 | 0.5 | 0.2 | 0.2 | 709 |
| KDYVVVHGYFTEVYY | 0.5 | 0.4 | 0.4 | 0.2 | 0.6 | 0.5 | 0.5 | 0.2 | 0.2 | 710 |
| DYVVVHGYFTEVYYQ | 0.6 | 0.5 | 0.5 | 0.3 | 0.7 | 0.7 | 0.5 | 0.3 | 0.2 | 711 |
| YVVVHGYFTEVYYQL | 0.6 | 0.5 | 0.5 | 0.3 | 0.4 | 0.4 | 0.4 | 0.2 | 0.2 | 712 |
| VVVHGYFTEVYYQLE | 0.6 | 0.5 | 0.5 | 0.3 | 0.5 | 0.5 | 0.4 | 0.3 | 0.2 | 713 |
| VVHGYFTEVYYQLES | 0.6 | 0.4 | 0.4 | 0.3 | 0.6 | 0.5 | 0.4 | 0.2 | 0.2 | 714 |
| VHGYFTEVYYQLEST | 0.5 | 0.4 | 0.5 | 0.3 | 0.5 | 0.6 | 0.5 | 0.3 | 0.2 | 715 |
| HGYFTEVYYQLESTQ | 0.5 | 0.4 | 0.4 | 0.3 | 0.5 | 0.5 | 0.4 | 0.3 | 0.2 | 716 |
| GYFTEVYYQLESTQI | 0.6 | 0.5 | 0.5 | 0.4 | 0.6 | 0.6 | 0.4 | 0.4 | 0.2 | 717 |
| YFTEVYYQLESTQIT | 0.5 | 0.5 | 0.3 | 0.2 | 0.4 | 0.4 | 0.3 | 0.2 | 0.2 | 718 |
| FTEVYYQLESTQITT | 0.5 | 0.5 | 0.4 | 0.4 | 0.7 | 0.5 | 0.4 | 0.3 | 0.2 | 719 |
| TEVYYQLESTQITTD | 0.5 | 0.5 | 0.2 | 0.3 | 0.5 | 0.4 | 0.3 | 0.3 | 0.2 | 720 |
| EVYYQLESTQITTDT | 0.5 | 0.4 | 0.1 | 0.2 | 0.5 | 0.5 | 0.3 | 0.3 | 0.2 | 721 |
| VYYQLESTQITTDTG | 0.4 | 0.2 | 0.1 | 0.1 | 0.4 | 0.3 | 0.1 | 0.2 | 0.2 | 722 |
| YYQLESTQITTDTGI | 0.4 | 0.3 | 0.2 | 0.2 | 0.5 | 0.4 | 0.3 | 0.3 | 0.2 | 723 |
| YQLESTQITTDTGIE | 0.4 | 0.3 | 0.1 | 0.1 | 0.3 | 0.3 | 0.2 | 0.2 | 0.1 | 724 |
| QLESTQITTDTGIEN | 0.4 | 0.3 | 0.3 | 0.2 | 0.5 | 0.5 | 0.4 | 0.4 | 0.2 | 725 |
| LESTQITTDTGIENA | 0.4 | 0.3 | 0.2 | 0.2 | 0.3 | 0.4 | 0.3 | 0.2 | 0.2 | 726 |
| ESTQITTDTGIENAT | 0.4 | 0.2 | 0.1 | 0.2 | 0.3 | 0.3 | 0.3 | 0.1 | 0.2 | 727 |
| STQITTDTGIENATF | 0.6 | 0.4 | 0.3 | 0.3 | 0.6 | 0.6 | 0.4 | 0.4 | 0.2 | 728 |
| TQITTDTGIENATFF | 0.5 | 0.3 | 0.2 | 0.3 | 0.4 | 0.5 | 0.4 | 0.3 | 0.2 | 729 |
| QITTDTGIENATFFI | 0.7 | 0.7 | 0.6 | 0.5 | 1.0 | 0.9 | 0.5 | 0.5 | 0.3 | 730 |
| ITTDTGIENATFFIF | 0.7 | 0.6 | 0.6 | 0.5 | 0.9 | 0.8 | 0.7 | 0.6 | 0.3 | 731 |
| TTDTGIENATFFIFN | 0.6 | 0.6 | 0.7 | 0.6 | 1.0 | 0.9 | 0.5 | 0.6 | 0.3 | 732 |
| TDTGIENATFFIFNK | 0.5 | 0.5 | 0.5 | 0.5 | 0.7 | 0.7 | 0.5 | 0.4 | 0.3 | 733 |
| DTGIENATFFIFNKL | 0.6 | 0.5 | 0.6 | 0.2 | 0.7 | 0.6 | 0.6 | 0.4 | 0.2 | 734 |
| TGIENATFFIFNKLV | 0.6 | 0.5 | 0.6 | 0.5 | 0.7 | 0.7 | 0.4 | 0.2 | 0.3 | 735 |
| GIENATFFIFNKLVK | 0.6 | 0.4 | 0.5 | 0.5 | 0.6 | 0.6 | 0.5 | 0.2 | 0.2 | 736 |
| IENATFFIFNKLVKD | 0.5 | 0.4 | 0.4 | 0.2 | 0.5 | 0.5 | 0.3 | 0.1 | 0.2 | 737 |
| ENATFFIFNKLVKDP | 0.4 | 0.3 | 0.5 | 0.4 | 0.4 | 0.5 | 0.2 | 0.2 | 0.2 | 738 |
| NATFFIFNKLVKDPP | 0.5 | 0.3 | 0.3 | 0.4 | 0.4 | 0.4 | 0.0 | 0.2 | 0.2 | 739 |
| 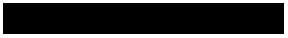 | 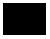 | 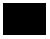 | 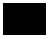 | 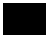 | 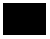 | 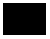 | 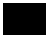 | 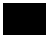 | 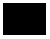 | 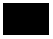 |
| 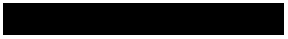 | 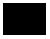 | 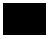 | 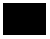 | 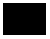 | 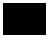 | 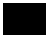 | 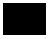 | 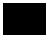 | 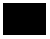 | 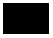 |
| 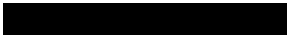 | 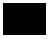 | 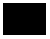 | 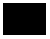 | 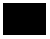 | 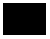 | 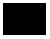 | 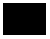 | 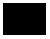 | 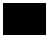 | 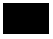 |
| 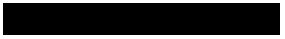 | 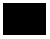 | 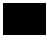 | 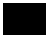 | 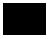 | 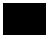 | 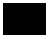 | 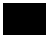 | 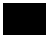 | 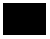 | 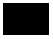 |
| 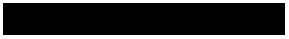 | 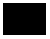 | 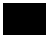 | 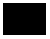 | 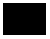 | 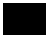 | 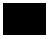 | 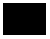 | 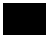 | 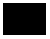 | 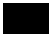 |
| 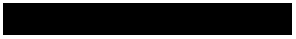 | 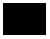 | 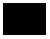 | 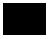 | 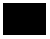 | 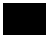 | 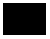 | 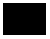 | 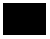 | 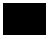 | 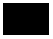 |
| 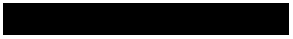 | 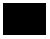 | 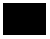 | 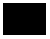 | 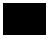 | 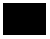 | 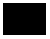 | 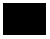 | 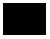 | 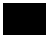 | 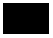 |
| 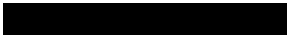 | 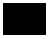 | 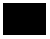 | 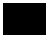 | 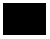 | 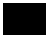 | 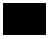 | 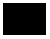 | 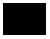 | 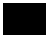 | 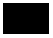 |
| 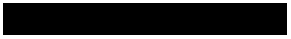 | 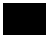 | 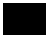 | 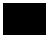 | 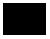 | 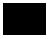 | 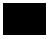 | 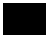 | 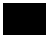 | 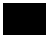 | 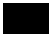 |
| 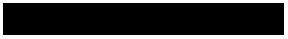 | 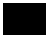 | 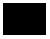 | 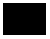 | 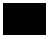 | 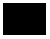 | 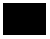 | 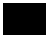 | 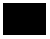 | 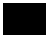 | 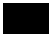 |
| 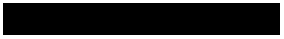 | 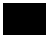 | 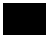 | 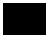 | 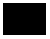 | 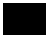 | 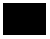 | 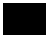 | 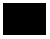 | 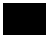 | 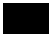 |
| DPPNVQIHTIDGSSG | 0.4 | 0.2 | 0.2 | 0.2 | 0.3 | 0.3 | 0.2 | 0.2 | 0.2 | 740 |
| PPNVQIHTIDGSSGV | 0.5 | 0.4 | 0.3 | 0.4 | 0.6 | 0.5 | 0.3 | 0.4 | 0.2 | 741 |
| PNVQIHTIDGSSGVA | 0.4 | 0.3 | 0.2 | 0.2 | 0.3 | 0.3 | 0.2 | 0.2 | 0.2 | 742 |
| NVQIHTIDGSSGVAN | 0.2 | 0.2 | 0.1 | 0.1 | 0.3 | 0.3 | 0.1 | 0.2 | 0.2 | 743 |
| VQIHTIDGSSGVANP | 0.4 | 0.4 | 0.3 | 0.3 | 0.4 | 0.5 | 0.1 | 0.3 | 0.2 | 744 |
| QIHTIDGSSGVANPA | 0.4 | 0.3 | 0.4 | 0.2 | 0.4 | 0.3 | 0.1 | 0.2 | 0.2 | 745 |
| IHTIDGSSGVANPAM | 0.5 | 0.4 | 0.3 | 0.2 | 0.3 | 0.3 | 0.2 | 0.3 | 0.2 | 746 |
| HTIDGSSGVANPAMD | 0.3 | 0.2 | 0.1 | 0.1 | 0.3 | 0.2 | 0.1 | 0.1 | 0.1 | 747 |
| TIDGSSGVANPAMDP | 0.5 | 0.4 | 0.4 | 0.3 | 0.3 | 0.3 | 0.2 | 0.3 | 0.2 | 748 |
| IDGSSGVANPAMDPI | 0.8 | 0.7 | 0.5 | 0.3 | 0.7 | 0.6 | 0.4 | 0.6 | 0.3 | 749 |
| 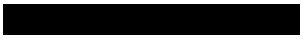 | 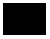 | 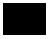 | 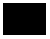 | 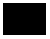 | 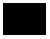 | 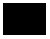 | 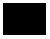 | 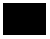 | 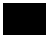 | 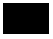 |
| 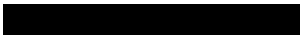 | 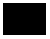 | 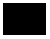 | 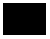 | 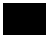 | 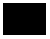 | 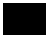 | 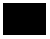 | 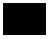 | 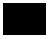 | 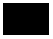 |
| 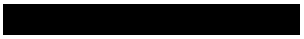 | 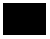 | 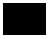 | 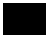 | 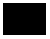 | 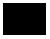 | 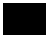 | 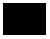 | 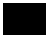 | 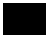 | 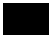 |
| 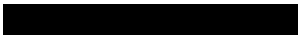 | 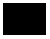 | 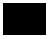 | 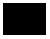 | 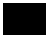 | 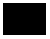 | 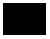 | 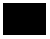 | 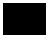 | 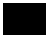 | 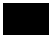 |
| 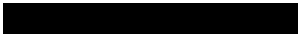 | 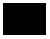 | 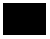 | 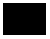 | 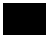 | 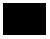 | 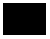 | 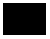 | 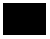 | 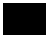 | 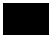 |
| 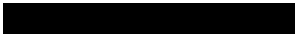 | 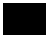 | 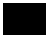 | 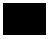 | 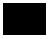 | 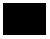 | 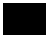 | 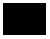 | 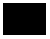 | 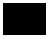 | 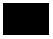 |
| 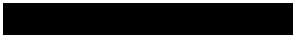 | 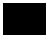 | 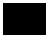 | 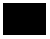 | 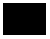 | 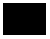 | 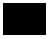 | 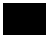 | 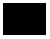 | 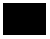 | 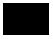 |
| 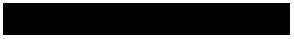 | 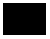 | 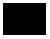 | 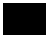 | 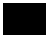 | 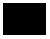 | 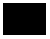 | 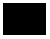 | 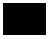 | 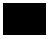 | 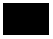 |
| 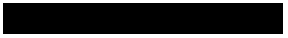 | 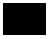 | 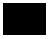 | 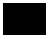 | 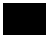 | 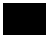 | 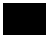 | 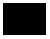 | 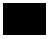 | 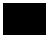 | 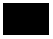 |
| 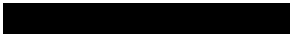 | 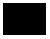 | 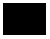 | 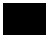 | 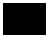 | 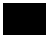 | 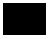 | 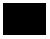 | 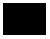 | 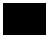 | 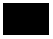 |
| 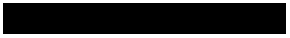 | 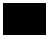 | 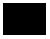 | 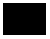 | 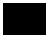 | 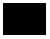 | 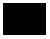 | 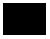 | 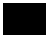 | 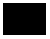 | 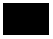 |
| 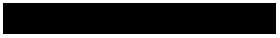 | 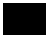 | 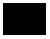 | 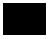 | 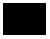 | 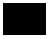 | 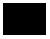 | 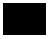 | 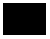 | 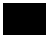 | 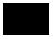 |

TABLE 3
Binding of the sera called SARS-yellow, SARS-green, 1a,
1b, 2, 6, 37, 62 and London to linear peptides of protein X2 of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| 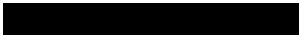 | 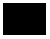 | 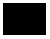 | 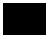 | 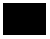 | 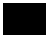 | 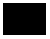 | 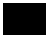 | 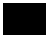 | 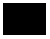 | 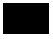 |
| 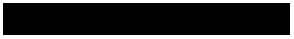 | 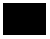 | 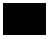 | 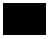 | 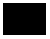 | 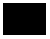 | 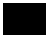 | 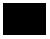 | 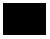 | 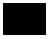 | 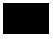 |
| 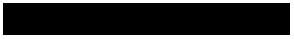 | 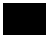 | 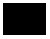 | 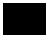 | 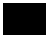 | 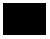 | 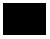 | 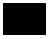 | 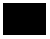 | 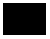 | 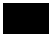 |
| 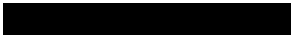 | 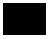 | 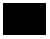 | 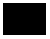 | 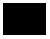 | 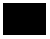 | 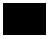 | 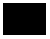 | 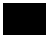 | 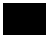 | 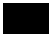 |
| 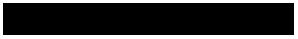 | 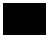 | 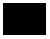 | 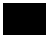 | 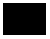 | 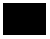 | 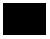 | 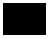 | 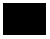 | 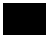 | 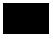 |
| 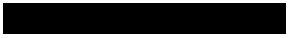 | 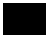 | 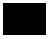 | 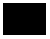 | 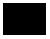 | 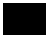 | 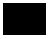 | 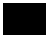 | 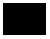 | 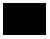 | 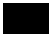 |
| 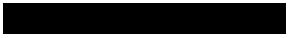 | 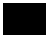 | 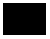 | 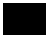 | 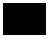 | 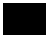 | 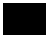 | 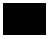 | 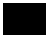 | 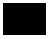 | 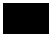 |
| 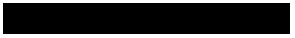 | 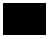 | 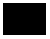 | 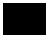 | 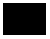 | 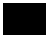 | 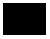 | 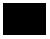 | 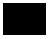 | 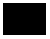 | 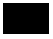 |
| 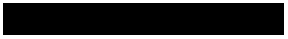 | 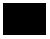 | 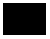 | 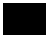 | 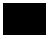 | 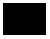 | 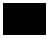 | 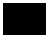 | 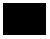 | 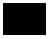 | 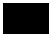 |
| THITMTTVYHITVSQ | 0.6 | 0.5 | 0.4 | 0.6 | 0.7 | 0.4 | 0.4 | 0.5 | 0.4 | 750 |
| HITMTTVYHITVSQI | 0.6 | 0.6 | 0.5 | 0.4 | 0.6 | 0.3 | 0.4 | 0.7 | 0.2 | 751 |
| ITMTTVYHITVSQIQ | 0.7 | 0.2 | 0.3 | 0.5 | 0.7 | 0.4 | 0.5 | 0.0 | 0.2 | 752 |
| TMTTVYHITVSQIQL | 0.6 | 0.2 | 0.4 | 0.5 | 0.7 | 0.3 | 0.4 | 0.7 | 0.2 | 753 |
| MTTVYHITVSQIQLS | 0.7 | 0.6 | 0.5 | 0.5 | 0.6 | 0.5 | 0.5 | 0.6 | 0.2 | 754 |
| TTVYHITVSQIQLSL | 0.7 | 0.5 | 0.5 | 0.5 | 0.7 | 0.6 | 0.4 | 0.8 | 0.1 | 755 |
| TVYHITVSQIQLSLL | 0.6 | 0.5 | 0.5 | 0.4 | 0.6 | 0.4 | 0.4 | 0.7 | 0.1 | 756 |
| VYHITVSQIQLSLLK | 0.6 | 0.5 | 0.5 | 0.5 | 0.7 | 0.4 | 0.4 | 0.7 | 0.2 | 757 |
| YHITVSQIQLSLLKV | 0.6 | 0.5 | 0.5 | 0.5 | 0.7 | 0.5 | 0.4 | 0.6 | 0.1 | 758 |
| HITVSQIQLSLLKVT | 0.6 | 0.5 | 0.5 | 0.5 | 0.7 | 0.5 | 0.4 | 0.8 | 0.2 | 759 |
| ITVSQIQLSLLKVTA | 0.6 | 0.5 | 0.4 | 0.5 | 0.6 | 0.4 | 0.4 | 0.7 | 0.1 | 760 |
| TVSQIQLSLLKVTAF | 0.5 | 0.4 | 0.4 | 0.5 | 0.6 | 0.4 | 0.3 | 0.6 | 0.1 | 761 |
| VSQIQLSLLKVTAFQ | 0.5 | 0.4 | 0.4 | 0.5 | 0.6 | 0.3 | 0.3 | 0.4 | 0.1 | 762 |
| SQIQLSLLKVTAFQH | 0.6 | 0.5 | 0.4 | 0.5 | 0.5 | 0.4 | 0.3 | 0.3 | 0.2 | 763 |
| QIQLSLLKVTAFQHQ | 0.5 | 0.4 | 0.4 | 0.4 | 0.6 | 0.3 | 0.3 | 0.4 | 0.2 | 764 |
| IQLSLLKVTAFQHQN | 0.5 | 0.4 | 0.4 | 0.5 | 0.6 | 0.3 | 0.3 | 0.3 | 0.2 | 765 |
| QLSLLKVTAFQHQNS | 0.5 | 0.4 | 0.3 | 0.4 | 0.5 | 0.3 | 0.3 | 0.1 | 0.1 | 766 |
| LSLLKVTAFQHQNSK | 0.4 | 0.1 | 0.1 | 0.2 | 0.4 | 0.2 | 0.2 | 0.0 | 0.1 | 767 |
| SLLKVTAFQHQNSKK | 0.4 | 0.0 | 0.1 | 0.0 | 0.4 | 0.1 | 0.1 | 0.0 | 0.0 | 768 |
| LLKVTAFQHQNSKKT | 0.2 | 0.0 | 0.1 | 0.2 | 0.5 | 0.1 | 0.2 | 0.1 | 0.1 | 769 |
| LKVTAFQHQNSKKTT | 0.5 | 0.4 | 0.3 | 0.3 | 0.5 | 0.3 | 0.2 | 0.7 | 0.2 | 770 |
| KVTAFQHQNSKKTTK | 0.3 | 0.2 | 0.2 | 0.2 | 0.3 | 0.2 | 0.1 | 0.4 | 0.0 | 771 |
| VTAFQHQNSKKTTKL | 0.4 | 0.4 | 0.2 | 0.3 | 0.5 | 0.3 | 0.1 | 0.7 | 0.1 | 772 |
| TAFQHQNSKKTTKLV | 0.7 | 0.5 | 0.5 | 0.7 | 0.7 | 0.4 | 0.4 | 0.8 | 0.1 | 511 |
| AFQHQNSKKTTKLVV | 0.6 | 0.5 | 0.6 | 0.9 | 0.8 | 0.4 | 0.4 | 0.7 | 0.2 | 512 |
| 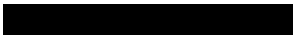 | 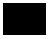 | 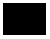 | 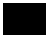 | 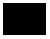 | 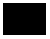 | 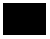 | 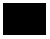 | 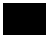 | 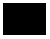 | 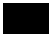 |
| 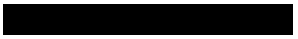 | 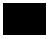 | 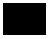 | 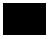 | 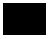 | 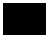 | 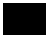 | 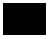 | 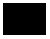 | 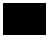 | 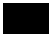 |
| 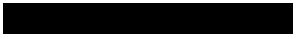 | 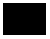 | 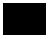 | 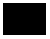 | 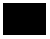 | 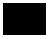 | 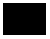 | 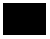 | 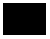 | 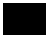 | 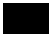 |
| 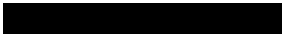 | 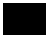 | 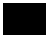 | 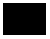 | 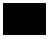 | 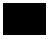 | 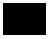 | 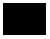 | 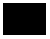 | 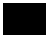 | 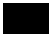 |
| 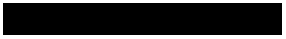 | 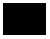 | 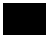 | 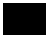 | 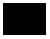 | 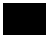 | 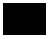 | 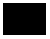 | 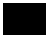 | 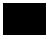 | 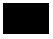 |
| 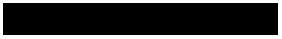 | 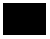 | 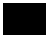 | 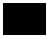 | 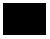 | 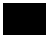 | 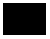 | 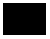 | 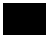 | 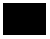 | 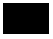 |
| 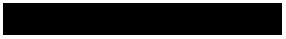 | 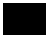 | 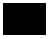 | 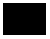 | 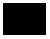 | 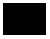 | 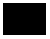 | 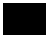 | 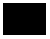 | 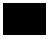 | 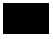 |
| 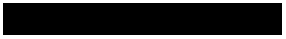 | 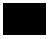 | 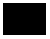 | 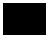 | 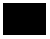 | 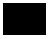 | 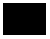 | 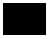 | 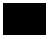 | 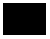 | 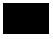 |
| 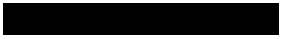 | 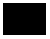 | 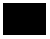 | 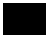 | 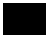 | 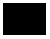 | 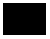 | 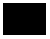 | 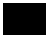 | 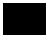 | 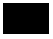 |
| 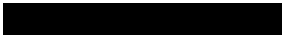 | 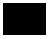 | 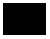 | 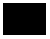 | 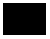 | 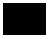 | 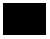 | 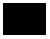 | 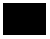 | 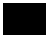 | 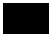 |
| 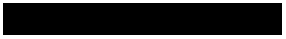 | 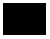 | 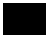 | 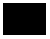 | 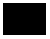 | 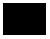 | 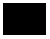 | 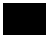 | 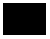 | 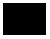 | 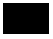 |
| LVVILRIGTQVLKTM | 0.7 | 0.5 | 0.5 | 0.4 | 0.6 | 0.7 | 0.5 | 0.7 | 0.5 | 773 |
| VVILRIGTQVLKTMS | 0.6 | 0.5 | 0.5 | 0.5 | 0.6 | 0.6 | 0.4 | 0.6 | 0.5 | 774 |
| VILRIGTQVLKTMSL | 0.6 | 0.5 | 0.5 | 0.5 | 0.6 | 0.6 | 0.4 | 0.4 | 0.4 | 775 |
| ILRIGTQVLKTMSLY | 0.7 | 0.5 | 0.4 | 0.4 | 0.6 | 0.6 | 0.4 | 0.5 | 0.4 | 776 |
| 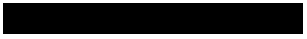 | 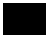 | 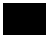 | 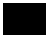 | 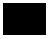 | 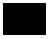 | 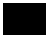 | 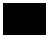 | 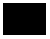 | 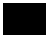 | 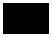 |
| 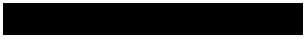 | 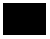 | 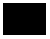 | 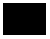 | 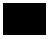 | 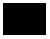 | 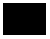 | 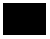 | 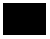 | 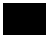 | 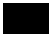 |
| 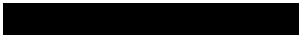 | 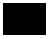 | 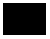 | 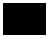 | 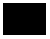 | 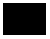 | 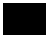 | 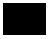 | 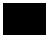 | 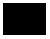 | 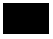 |
| 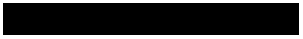 | 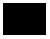 | 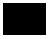 | 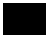 | 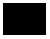 | 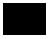 | 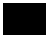 | 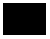 | 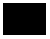 | 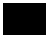 | 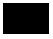 |
| 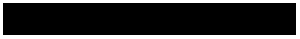 | 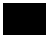 | 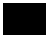 | 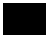 | 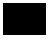 | 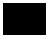 | 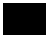 | 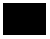 | 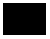 | 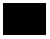 | 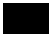 |
| 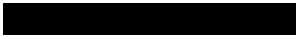 | 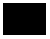 | 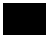 | 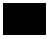 | 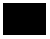 | 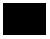 | 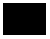 | 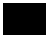 | 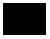 | 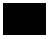 | 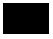 |
| 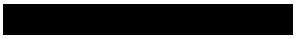 | 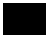 | 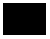 | 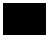 | 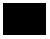 | 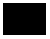 | 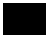 | 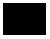 | 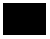 | 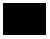 | 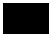 |
| 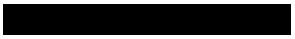 | 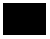 | 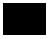 | 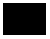 | 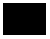 | 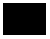 | 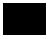 | 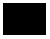 | 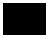 | 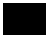 | 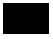 |
| 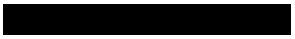 | 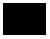 | 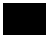 | 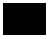 | 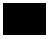 | 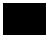 | 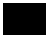 | 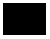 | 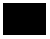 | 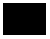 | 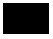 |
| TMSLYMAISPKFTTS | 0.5 | 0.0 | 0.4 | 0.3 | 0.2 | 0.7 | 0.4 | 0.0 | 0.5 | 777 |
| MSLYMAISPKFTTSL | 0.8 | 0.7 | 0.4 | 0.3 | 0.6 | 0.6 | 0.4 | 0.3 | 0.5 | 778 |
| SLYMAISPKFTTSLS | 0.7 | 0.5 | 0.5 | 0.4 | 0.6 | 0.6 | 0.6 | 0.6 | 0.6 | 779 |
| LYMAISPKFTTSLSL | 0.7 | 0.5 | 0.5 | 0.3 | 0.6 | 0.6 | 0.3 | 0.6 | 0.5 | 780 |
| YMAISPKFTTSLSLH | 0.7 | 0.6 | 0.4 | 0.4 | 0.6 | 0.6 | 0.4 | 0.6 | 0.5 | 781 |
| MAISPKFTTSLSLHK | 0.7 | 0.7 | 0.7 | 0.6 | 0.7 | 0.7 | 0.5 | 0.5 | 0.5 | 782 |
| AISPKFTTSLSLHKL | 0.6 | 0.5 | 0.5 | 0.6 | 0.6 | 0.6 | 0.5 | 0.5 | 0.4 | 783 |
| ISPKFTTSLSLHKLL | 0.5 | 0.4 | 0.4 | 0.5 | 0.5 | 0.6 | 0.4 | 0.4 | 0.5 | 784 |
| SPKFTTSLSLHKLLQ | 0.6 | 0.4 | 0.4 | 0.4 | 0.5 | 0.5 | 0.4 | 0.3 | 0.5 | 785 |
| PKFTTSLSLHKLLQT | 0.6 | 0.4 | 0.5 | 0.3 | 0.5 | 0.6 | 0.4 | 0.3 | 0.5 | 786 |
| KFTTSLSLHKLLQTL | 0.5 | 0.4 | 0.4 | 0.6 | 0.5 | 0.6 | 0.5 | 0.5 | 0.4 | 787 |
| FTTSLSLHKLLQTLV | 0.5 | 0.5 | 0.4 | 0.6 | 0.5 | 0.5 | 0.4 | 0.4 | 0.5 | 788 |
| TTSLSLHKLLQTLVL | 0.5 | 0.5 | 0.3 | 0.5 | 0.5 | 0.5 | 0.5 | 0.3 | 0.6 | 789 |
| TSLSLHKLLQTLVLK | 0.7 | 0.6 | 0.5 | 1.0 | 0.6 | 0.7 | 0.6 | 0.6 | 0.7 | 790 |
| SLSLHKLLQTLVLKM | 0.6 | 0.5 | 0.3 | 0.5 | 0.4 | 0.5 | 0.4 | 0.3 | 0.6 | 791 |
| LSLHKLLQTLVLKML | 0.7 | 0.4 | 0.3 | 0.3 | 0.4 | 0.5 | 0.4 | 0.2 | 0.4 | 792 |
| SLHKLLQTLVLKMLH | 0.7 | 0.5 | 0.3 | 0.3 | 0.4 | 0.6 | 0.3 | 0.1 | 0.4 | 793 |
| LHKLLQTLVLKMLHS | 0.6 | 0.0 | 0.4 | 0.3 | 0.4 | 0.5 | 0.4 | 0.3 | 0.6 | 794 |
| HKLLQTLVLKMLHSS | 0.5 | 0.5 | 0.4 | 0.2 | 0.5 | 0.5 | 0.4 | 0.5 | 0.4 | 795 |
| KLLQTLVLKMLHSSS | 0.6 | 0.6 | 0.4 | 0.3 | 0.5 | 0.6 | 0.4 | 0.4 | 0.5 | 796 |
| LLQTLVLKMLHSSSL | 0.6 | 0.5 | 0.3 | 0.3 | 0.5 | 0.5 | 0.4 | 0.5 | 0.5 | 797 |
| LQTLVLKMLHSSSLT | 0.7 | 0.5 | 0.5 | 0.4 | 0.6 | 0.6 | 0.4 | 0.7 | 0.5 | 798 |
| QTLVLKMLHSSSLTS | 0.7 | 0.5 | 0.5 | 0.5 | 0.5 | 0.6 | 0.4 | 0.7 | 0.5 | 799 |
| TLVLKMLHSSSLTSL | 0.7 | 0.5 | 0.4 | 0.4 | 0.5 | 0.6 | 0.4 | 0.4 | 0.4 | 800 |
| LVLKMLHSSSLTSLL | 0.6 | 0.4 | 0.3 | 0.3 | 0.4 | 0.5 | 0.3 | 0.4 | 0.5 | 801 |
| VLKMLHSSSLTSLLK | 0.6 | 0.5 | 0.5 | 0.5 | 0.5 | 0.6 | 0.4 | 0.5 | 0.5 | 802 |
| LKMLHSSSLTSLLKT | 0.6 | 0.5 | 0.6 | 0.4 | 0.6 | 0.6 | 0.5 | 0.6 | 0.5 | 803 |
| KMLHSSSLTSLLKTH | 0.6 | 0.5 | 0.5 | 0.6 | 0.5 | 0.6 | 0.9 | 0.4 | 0.6 | 804 |
| MLHSSSLTSLLKTHR | 0.6 | 0.4 | 0.4 | 0.6 | 0.5 | 0.5 | 0.6 | 0.5 | 0.5 | 805 |
| LHSSSLTSLLKTHRM | 0.6 | 0.5 | 0.4 | 0.5 | 0.5 | 0.5 | 0.6 | 0.6 | 0.6 | 806 |
| HSSSLTSLLKTHRMC | 0.6 | 0.5 | 0.3 | 0.5 | 0.5 | 0.6 | 0.5 | 0.4 | 0.5 | 807 |
| SSSLTSLLKTHRMCK | 0.6 | 0.5 | 0.4 | 0.8 | 0.6 | 0.7 | 0.6 | 0.2 | 0.6 | 808 |
| SSLTSLLKTHRMCKY | 0.6 | 0.5 | 0.4 | 0.5 | 0.5 | 0.6 | 0.4 | 0.3 | 0.5 | 809 |
| SLTSLLKTHRMCKYT | 0.8 | 0.4 | 0.4 | 0.4 | 0.3 | 0.7 | 0.4 | 0.3 | 0.5 | 810 |
| LTSLLKTHRMCKYTQ | 0.7 | 0.3 | 0.5 | 0.5 | 0.6 | 0.6 | 0.5 | 0.4 | 0.2 | 811 |
| TSLLKTHRMCKYTQS | 0.7 | 0.7 | 0.6 | 0.5 | 0.7 | 0.7 | 0.5 | 0.8 | 0.6 | 812 |
| SLLKTHRMCKYTQST | 0.8 | 0.6 | 0.6 | 0.6 | 0.6 | 0.7 | 0.6 | 0.7 | 0.7 | 813 |
| LLKTHRMCKYTQSTA | 0.8 | 0.5 | 0.6 | 0.6 | 0.7 | 0.7 | 0.5 | 0.8 | 0.6 | 814 |
| LKTHRMCKYTQSTAL | 0.8 | 0.6 | 0.5 | 0.6 | 0.7 | 0.7 | 0.5 | 0.8 | 0.5 | 815 |
| KTHRMCKYTQSTALQ | 0.7 | 0.5 | 0.6 | 0.5 | 0.7 | 0.6 | 0.5 | 0.8 | 0.5 | 816 |
| THRMCKYTQSTALQE | 0.9 | 0.8 | 0.5 | 0.6 | 0.7 | 0.8 | 0.7 | 0.8 | 0.6 | 817 |
| HRMCKYTQSTALQEL | 0.9 | 0.8 | 0.5 | 0.5 | 0.8 | 0.8 | 0.5 | 0.8 | 0.8 | 818 |
| RMCKYTQSTALQELL | 0.8 | 0.7 | 0.5 | 0.4 | 0.7 | 0.8 | 0.5 | 0.6 | 0.6 | 819 |
| MCKYTQSTALQELLI | 0.8 | 0.7 | 0.6 | 0.5 | 0.6 | 0.8 | 0.5 | 0.7 | 0.8 | 820 |
| CKYTQSTALQELLIQ | 0.7 | 0.5 | 0.5 | 0.4 | 0.7 | 0.7 | 0.5 | 0.6 | 0.7 | 821 |
| KYTQSTALQELLIQQ | 0.7 | 0.6 | 0.4 | 0.5 | 0.6 | 0.6 | 0.5 | 0.6 | 0.6 | 822 |
| YTQSTALQELLIQQW | 0.6 | 0.5 | 0.4 | 0.4 | 0.5 | 0.6 | 0.5 | 0.6 | 0.6 | 823 |
| TQSTALQELLIQQWI | 0.7 | 0.6 | 0.3 | 0.7 | 0.6 | 0.6 | 0.7 | 0.7 | 0.6 | 824 |
| QSTALQELLIQQWIQ | 0.6 | 0.4 | 0.4 | 0.4 | 0.5 | 0.5 | 0.5 | 0.4 | 0.6 | 825 |
| STALQELLIQQWIQF | 0.6 | 0.4 | 0.3 | 0.3 | 0.5 | 0.6 | 0.4 | 0.3 | 0.5 | 826 |
| TALQELLIQQWIQFM | 0.8 | 0.6 | 0.3 | 0.2 | 0.5 | 0.5 | 0.4 | 0.4 | 0.5 | 827 |
| ALQELLIQQWIQFMM | 0.7 | 0.6 | 0.4 | 0.4 | 0.6 | 0.6 | 0.5 | 0.4 | 0.7 | 828 |
| LQELLIQQWIQFMMS | 0.7 | 0.6 | 0.3 | 0.3 | 0.5 | 0.5 | 0.4 | 0.6 | 0.5 | 829 |
| QELLIQQWIQFMMSR | 0.7 | 0.4 | 0.4 | 0.3 | 0.5 | 0.5 | 0.4 | 0.6 | 0.6 | 830 |
| ELLIQQWIQFMMSRR | 0.7 | 0.5 | 0.4 | 0.3 | 0.6 | 0.6 | 0.4 | 0.8 | 0.6 | 831 |
| LLIQQWIQFMMSRRR | 0.7 | 0.5 | 0.5 | 0.5 | 0.6 | 0.6 | 0.4 | 0.8 | 0.5 | 832 |
| LIQQWIQFMMSRRRL | 0.7 | 0.4 | 0.4 | 0.7 | 0.6 | 0.5 | 0.5 | 0.8 | 0.4 | 833 |
| IQQWIQFMMSRRRLL | 0.6 | 0.5 | 0.4 | 0.6 | 0.6 | 0.5 | 0.4 | 0.8 | 0.3 | 834 |
| QQWIQFMMSRRRLLA | 0.6 | 0.4 | 0.4 | 0.8 | 0.6 | 0.6 | 0.5 | 0.6 | 0.6 | 835 |
| QWIQFMMSRRRLLAC | 0.7 | 0.5 | 0.5 | 0.7 | 0.6 | 0.6 | 0.5 | 0.6 | 0.5 | 836 |
| WIQFMMSRRRLLACL | 0.6 | 0.4 | 0.4 | 0.5 | 0.5 | 0.6 | 0.5 | 0.7 | 0.5 | 837 |
| IQFMMSRRRLLACLC | 0.7 | 0.5 | 0.4 | 0.6 | 0.6 | 0.7 | 0.5 | 0.8 | 0.6 | 838 |
| QFMMSRRRLLACLCK | 0.7 | 0.5 | 0.5 | 0.6 | 0.5 | 0.6 | 0.6 | 0.4 | 0.4 | 839 |
| FMMSRRRLLACLCKH | 0.7 | 0.5 | 0.5 | 0.7 | 0.6 | 0.6 | 0.7 | 0.5 | 0.5 | 840 |
| 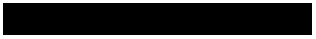 | 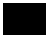 | 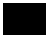 | 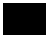 | 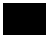 | 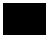 | 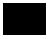 | 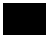 | 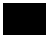 | 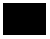 | 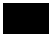 |
| 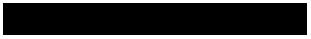 | 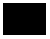 | 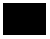 | 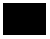 | 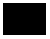 | 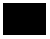 | 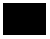 | 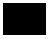 | 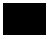 | 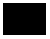 | 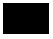 |
| 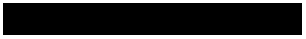 | 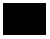 | 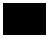 | 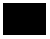 | 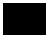 | 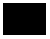 | 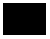 | 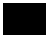 | 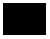 | 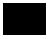 | 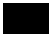 |
| 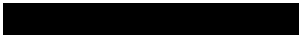 | 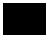 | 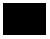 | 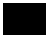 | 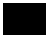 | 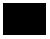 | 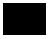 | 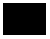 | 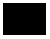 | 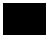 | 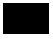 |
| 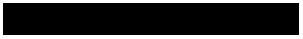 | 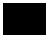 | 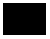 | 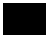 | 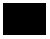 | 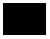 | 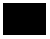 | 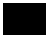 | 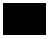 | 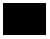 | 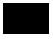 |
| 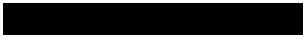 | 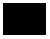 | 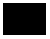 | 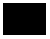 | 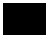 | 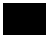 | 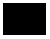 | 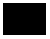 | 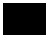 | 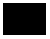 | 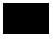 |
| 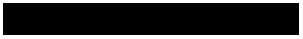 | 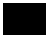 | 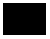 | 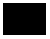 | 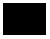 | 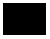 | 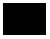 | 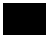 | 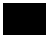 | 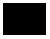 | 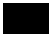 |
| 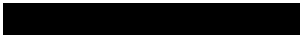 | 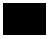 | 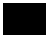 | 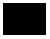 | 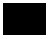 | 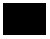 | 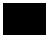 | 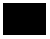 | 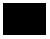 | 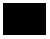 | 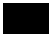 |
| 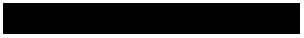 | 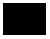 | 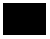 | 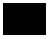 | 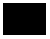 | 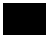 | 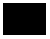 | 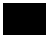 | 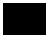 | 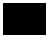 | 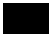 |
| 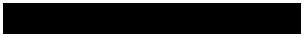 | 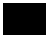 | 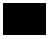 | 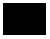 | 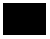 | 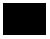 | 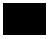 | 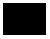 | 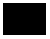 | 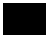 | 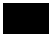 |
| 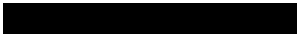 | 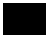 | 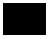 | 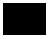 | 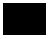 | 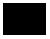 | 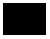 | 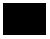 | 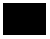 | 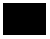 | 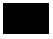 |
| 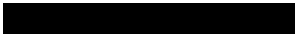 | 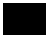 | 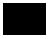 | 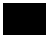 | 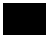 | 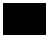 | 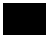 | 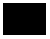 | 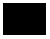 | 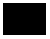 | 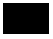 |
| 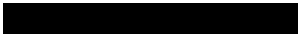 | 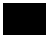 | 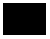 | 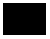 | 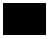 | 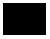 | 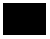 | 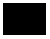 | 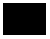 | 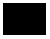 | 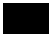 |
| 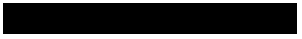 | 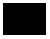 | 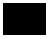 | 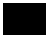 | 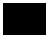 | 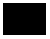 | 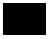 | 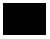 | 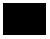 | 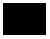 | 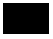 |
| 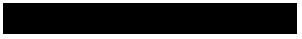 | 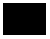 | 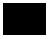 | 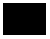 | 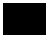 | 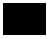 | 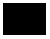 | 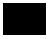 | 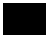 | 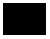 | 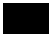 |
| 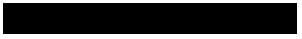 | 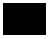 | 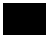 | 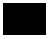 | 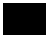 | 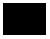 | 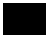 | 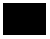 | 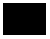 | 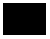 | 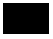 |
| 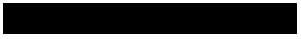 | 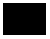 | 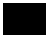 | 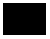 | 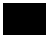 | 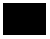 | 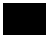 | 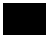 | 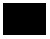 | 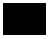 | 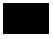 |
| 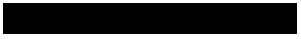 | 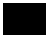 | 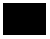 | 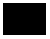 | 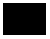 | 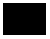 | 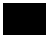 | 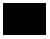 | 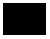 | 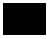 | 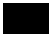 |

TABLE 4
Binding of the sera called SARS-yellow, SARS-green, 1a,
1b, 2, 6, 37, 62 and London to looped/cyclic peptides of
protein X2 of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| 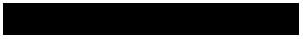 | 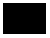 | 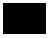 | 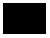 | 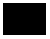 | 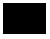 | 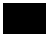 | 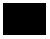 | 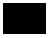 | 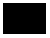 | 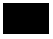 |
| 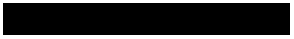 | 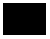 | 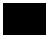 | 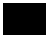 | 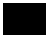 | 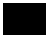 | 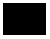 | 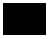 | 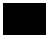 | 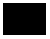 | 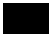 |
| 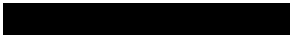 | 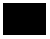 | 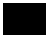 | 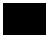 | 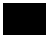 | 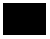 | 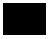 | 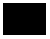 | 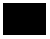 | 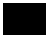 | 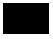 |
| 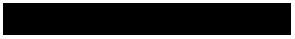 | 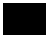 | 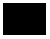 | 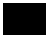 | 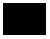 | 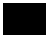 | 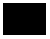 | 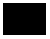 | 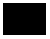 | 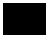 | 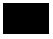 |
| 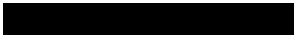 | 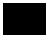 | 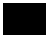 | 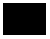 | 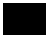 | 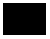 | 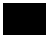 | 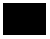 | 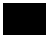 | 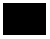 | 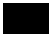 |
| 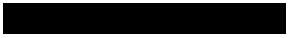 | 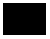 | 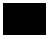 | 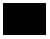 | 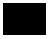 | 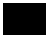 | 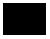 | 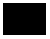 | 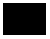 | 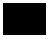 | 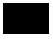 |
| 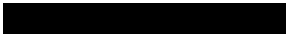 | 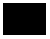 | 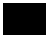 | 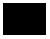 | 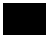 | 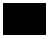 | 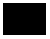 | 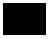 | 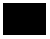 | 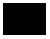 | 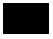 |
| 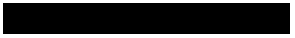 | 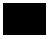 | 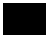 | 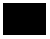 | 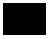 | 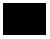 | 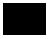 | 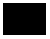 | 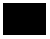 | 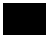 | 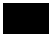 |
| 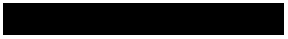 | 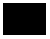 | 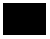 | 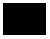 | 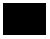 | 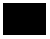 | 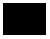 | 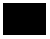 | 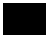 | 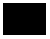 | 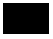 |
| THITMTTVYHITVSQ | 0.6 | 0.6 | 0.3 | 0.2 | 0.5 | 0.6 | 0.2 | 0.5 | 0.3 | 750 |
| HITMTTVYHITVSQI | 0.6 | 0.6 | 0.3 | 0.3 | 0.6 | 0.7 | 0.2 | 0.5 | 0.4 | 751 |
| ITMTTVYHITVSQIQ | 0.4 | 0.5 | 0.2 | 0.2 | 0.4 | 0.5 | 0.2 | 0.3 | 0.3 | 752 |
| TMTTVYHITVSQIQL | 0.4 | 0.4 | 0.3 | 0.2 | 0.4 | 0.5 | 0.2 | 0.3 | 0.3 | 753 |
| MTTVYHITVSQIQLS | 0.5 | 0.5 | 0.3 | 0.3 | 0.5 | 0.6 | 0.2 | 0.4 | 0.4 | 754 |
| TTVYHITVSQIQLSL | 0.5 | 0.5 | 0.3 | 0.3 | 0.4 | 0.5 | 0.2 | 0.4 | 0.2 | 755 |
| TVYHITVSQIQLSLL | 0.4 | 0.4 | 0.3 | 0.2 | 0.4 | 0.5 | 0.2 | 0.3 | 0.2 | 756 |
| VYHITVSQIQLSLLK | 0.5 | 0.2 | 0.2 | 0.6 | 0.4 | 0.6 | 1.3 | 0.3 | 0.3 | 757 |
| YHITVSQIQLSLLKV | 0.4 | 0.4 | 0.4 | 0.1 | 0.4 | 0.5 | 0.2 | 0.4 | 0.3 | 758 |
| HITVSQIQLSLLKVT | 0.5 | 0.5 | 0.5 | 0.4 | 0.5 | 0.6 | 0.8 | 0.6 | 0.4 | 759 |
| ITVSQIQLSLLKVTA | 0.5 | 0.5 | 0.4 | 1.4 | 0.5 | 0.5 | 1.4 | 0.6 | 0.3 | 760 |
| TVSQIQLSLLKVTAF | 0.5 | 0.5 | 0.4 | 1.0 | 0.4 | 0.6 | 1.4 | 0.7 | 0.4 | 761 |
| VSQIQLSLLKVTAFQ | 0.5 | 0.4 | 0.4 | 0.6 | 0.4 | 0.6 | 0.8 | 0.6 | 0.3 | 762 |
| SQIQLSLLKVTAFQH | 0.5 | 0.5 | 0.4 | 0.6 | 0.4 | 0.6 | 1.0 | 0.7 | 0.2 | 763 |
| QIQLSLLKVTAFQHQ | 0.6 | 0.5 | 0.4 | 0.6 | 0.4 | 0.6 | 0.7 | 0.6 | 0.4 | 764 |
| IQLSLLKVTAFQHQN | 0.5 | 0.6 | 0.3 | 0.4 | 0.4 | 0.6 | 0.2 | 0.5 | 0.2 | 765 |
| QLSLLKVTAFQHQNS | 0.5 | 0.5 | 0.3 | 0.3 | 0.4 | 0.6 | 0.3 | 0.3 | 0.2 | 766 |
| LSLLKVTAFQHQNSK | 0.3 | 0.2 | 0.1 | 0.1 | 0.2 | 0.3 | 0.1 | 0.2 | 0.1 | 767 |
| SLLKVTAFQHQNSKK | 0.3 | 0.2 | 0.1 | 0.1 | 0.2 | 0.4 | 0.1 | 0.2 | 0.1 | 768 |
| LLKVTAFQHQNSKKT | 0.4 | 0.3 | 0.2 | 0.2 | 0.3 | 0.4 | 0.2 | 0.3 | 0.3 | 769 |
| LKVTAFQHQNSKKTT | 0.3 | 0.2 | 0.1 | 0.1 | 0.2 | 0.4 | 0.1 | 0.2 | 0.1 | 770 |
| KVTAFQHQNSKKTTK | 0.2 | 0.2 | 0.0 | 0.0 | 0.2 | 0.3 | 0.1 | 0.1 | 0.0 | 771 |
| VTAFQHQNSKKTTKL | 0.4 | 0.3 | 0.1 | 0.3 | 0.3 | 0.5 | 0.2 | 0.5 | 0.2 | 772 |
| TAFQHQNSKKTTKLV | 0.3 | 0.3 | 0.2 | 0.3 | 0.2 | 0.4 | 0.2 | 0.3 | 0.1 | 511 |
| AFQHQNSKKTTKLVV | 0.5 | 0.5 | 0.4 | 0.4 | 0.5 | 0.6 | 0.2 | 0.5 | 0.3 | 512 |
| 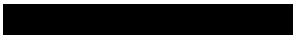 | 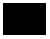 | 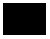 | 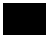 | 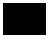 | 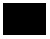 | 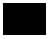 | 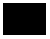 | 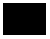 | 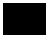 | 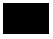 |
| 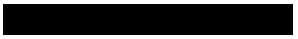 | 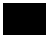 | 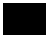 | 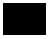 | 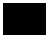 | 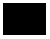 | 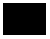 | 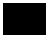 | 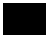 | 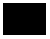 | 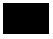 |
| 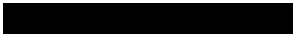 | 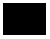 | 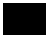 | 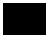 | 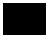 | 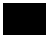 | 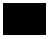 | 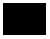 | 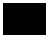 | 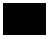 | 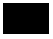 |
| 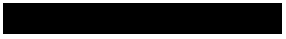 | 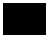 | 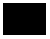 | 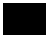 | 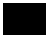 | 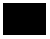 | 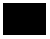 | 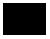 | 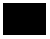 | 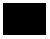 | 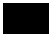 |
| 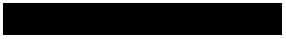 | 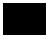 | 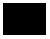 | 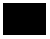 | 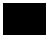 | 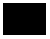 | 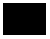 | 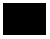 | 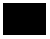 | 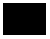 | 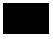 |
| 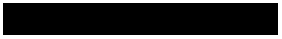 | 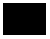 | 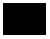 | 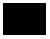 | 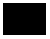 | 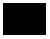 | 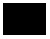 | 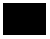 | 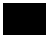 | 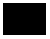 | 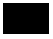 |
| 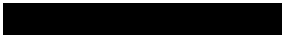 | 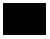 | 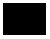 | 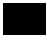 | 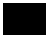 | 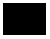 | 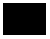 | 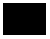 | 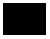 | 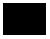 | 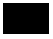 |
| 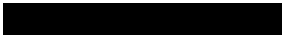 | 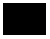 | 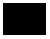 | 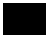 | 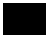 | 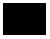 | 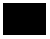 | 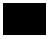 | 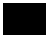 | 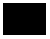 | 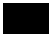 |
| 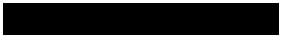 | 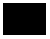 | 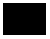 | 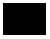 | 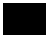 | 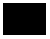 | 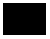 | 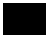 | 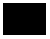 | 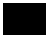 | 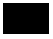 |
| 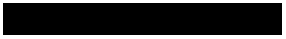 | 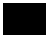 | 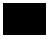 | 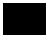 | 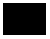 | 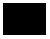 | 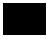 | 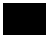 | 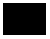 | 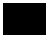 | 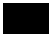 |
| 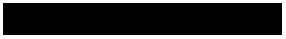 | 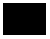 | 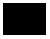 | 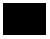 | 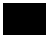 | 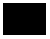 | 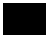 | 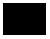 | 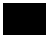 | 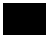 | 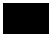 |
| LVVILRIGTQVLKTM | 0.6 | 0.6 | 0.4 | 0.4 | 0.4 | 0.7 | 0.3 | 0.5 | 0.4 | 773 |
| VVILRIGTQVLKTMS | 0.4 | 0.4 | 0.3 | 0.3 | 0.3 | 0.4 | 0.2 | 0.4 | 0.3 | 774 |
| VILRIGTQVLKTMSL | 0.6 | 0.6 | 0.4 | 0.5 | 0.5 | 0.7 | 0.2 | 0.4 | 0.3 | 775 |
| ILRIGTQVLKTMSLY | 0.6 | 0.5 | 0.3 | 0.5 | 0.5 | 0.6 | 0.3 | 0.6 | 0.3 | 776 |
| 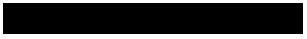 | 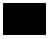 | 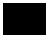 | 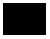 | 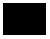 | 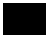 | 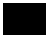 | 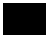 | 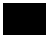 | 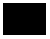 | 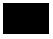 |
| 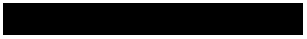 | 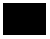 | 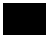 | 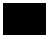 | 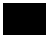 | 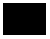 | 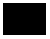 | 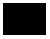 | 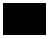 | 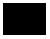 | 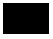 |
| 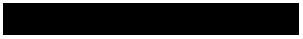 | 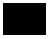 | 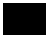 | 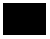 | 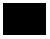 | 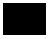 | 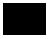 | 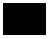 | 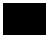 | 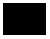 | 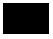 |
| 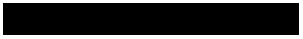 | 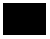 | 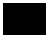 | 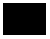 | 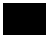 | 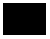 | 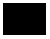 | 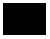 | 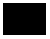 | 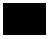 | 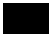 |
| 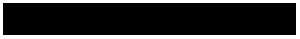 | 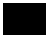 | 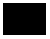 | 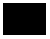 | 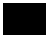 | 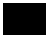 | 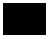 | 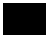 | 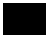 | 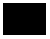 | 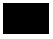 |
| 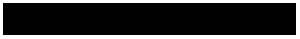 | 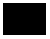 | 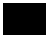 | 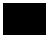 | 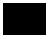 | 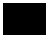 | 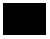 | 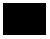 | 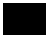 | 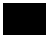 | 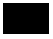 |
| 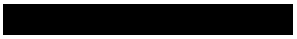 | 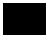 | 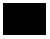 | 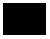 | 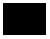 | 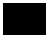 | 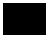 | 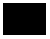 | 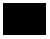 | 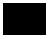 | 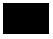 |
| 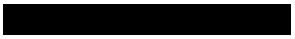 | 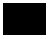 | 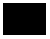 | 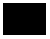 | 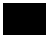 | 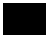 | 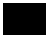 | 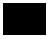 | 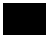 | 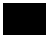 | 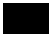 |
| 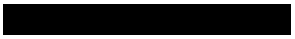 | 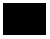 | 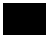 | 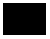 | 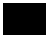 | 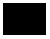 | 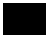 | 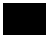 | 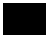 | 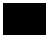 | 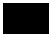 |
| TMSLYMAISPKFTTS | 0.6 | 0.6 | 0.4 | 0.4 | 0.5 | 0.7 | 0.2 | 0.6 | 0.4 | 777 |
| MSLYMAISPKFTTSL | 0.7 | 0.6 | 0.4 | 0.5 | 0.5 | 0.8 | 0.4 | 0.5 | 0.3 | 778 |
| SLYMAISPKFTTSLS | 0.6 | 0.6 | 0.3 | 0.4 | 0.4 | 0.5 | 0.3 | 0.6 | 0.5 | 779 |
| LYMAISPKFTTSLSL | 0.6 | 0.5 | 0.5 | 0.5 | 0.5 | 0.7 | 0.6 | 0.5 | 0.4 | 780 |
| YMAISPKFTTSLSLH | 0.6 | 0.6 | 0.4 | 0.5 | 0.5 | 0.6 | 0.4 | 0.4 | 0.4 | 781 |
| MAISPKFTTSLSLHK | 0.5 | 0.5 | 0.4 | 0.4 | 0.4 | 0.6 | 0.2 | 0.5 | 0.4 | 782 |
| AISPKFTTSLSLHKL | 0.6 | 0.6 | 0.4 | 0.8 | 0.5 | 0.7 | 1.1 | 0.4 | 0.3 | 783 |
| ISPKFTTSLSLHKLL | 0.5 | 0.5 | 0.4 | 0.4 | 0.4 | 0.7 | 0.3 | 0.3 | 0.2 | 784 |
| SPKFTTSLSLHKLLQ | 0.5 | 0.5 | 0.3 | 0.4 | 0.3 | 0.5 | 0.2 | 0.3 | 0.2 | 785 |
| PKFTTSLSLHKLLQT | 0.5 | 0.4 | 0.3 | 0.3 | 0.4 | 0.6 | 0.3 | 0.3 | 0.3 | 786 |
| KFTTSLSLHKLLQTL | 0.5 | 0.4 | 0.5 | 0.4 | 0.5 | 0.5 | 0.6 | 0.3 | 0.4 | 787 |
| FTTSLSLHKLLQTLV | 2.1 | 2.5 | 1.2 | 0.8 | 1.3 | 1.7 | 1.0 | 1.3 | 1.7 | 788 |
| TTSLSLHKLLQTLVL | 0.5 | 0.4 | 0.4 | 0.5 | 0.5 | 0.6 | 1.3 | 0.5 | 0.3 | 789 |
| TSLSLHKLLQTLVLK | 0.5 | 0.4 | 0.3 | 0.3 | 0.4 | 0.6 | 0.2 | 0.5 | 0.4 | 790 |
| SLSLHKLLQTLVLKM | 0.5 | 0.5 | 0.3 | 0.9 | 0.4 | 0.5 | 1.2 | 0.7 | 0.3 | 791 |
| LSLHKLLQTLVLKML | 0.5 | 0.4 | 0.3 | 0.6 | 0.4 | 0.6 | 1.3 | 0.6 | 0.2 | 792 |
| SLHKLLQTLVLKMLH | 0.6 | 0.5 | 0.4 | 0.3 | 0.4 | 0.7 | 0.3 | 0.5 | 0.3 | 793 |
| LHKLLQTLVLKMLHS | 0.6 | 0.5 | 0.4 | 0.6 | 0.4 | 0.6 | 1.0 | 0.7 | 0.4 | 794 |
| HKLLQTLVLKMLHSS | 0.6 | 0.6 | 0.4 | 0.4 | 0.5 | 0.7 | 0.3 | 0.5 | 0.3 | 795 |
| KLLQTLVLKMLHSSS | 0.5 | 0.5 | 0.3 | 0.5 | 0.4 | 0.6 | 0.4 | 0.4 | 0.3 | 796 |
| LLQTLVLKMLHSSSL | 0.5 | 0.5 | 0.3 | 0.7 | 0.4 | 0.6 | 1.1 | 0.5 | 0.3 | 797 |
| LQTLVLKMLHSSSLT | 0.4 | 0.4 | 0.3 | 0.5 | 0.3 | 0.4 | 0.5 | 0.3 | 0.3 | 798 |
| QTLVLKMLHSSSLTS | 0.6 | 0.4 | 0.3 | 0.5 | 0.4 | 0.6 | 0.9 | 0.4 | 0.3 | 799 |
| TLVLKMLHSSSLTSL | 0.7 | 0.6 | 0.5 | 1.0 | 0.5 | 0.7 | 1.0 | 0.5 | 0.3 | 800 |
| LVLKMLHSSSLTSLL | 0.6 | 0.5 | 0.3 | 0.4 | 0.4 | 0.6 | 0.2 | 0.4 | 0.3 | 801 |
| VLKMLHSSSLTSLLK | 0.4 | 0.2 | 0.1 | 0.1 | 0.2 | 0.4 | 0.1 | 0.2 | 0.2 | 802 |
| LKMLHSSSLTSLLKT | 0.5 | 0.5 | 0.3 | 0.3 | 0.4 | 0.6 | 0.2 | 0.2 | 0.3 | 803 |
| KMLHSSSLTSLLKTH | 0.3 | 0.4 | 0.2 | 0.2 | 0.2 | 0.3 | 0.2 | 0.3 | 0.7 | 804 |
| MLHSSSLTSLLKTHR | 0.5 | 0.5 | 0.5 | 0.2 | 0.3 | 0.5 | 0.2 | 0.5 | 0.4 | 805 |
| LHSSSLTSLLKTHRM | 0.4 | 0.4 | 0.3 | 0.2 | 0.3 | 0.4 | 0.2 | 0.3 | 0.3 | 806 |
| HSSSLTSLLKTHRMC | 0.4 | 0.4 | 0.3 | 0.2 | 0.3 | 0.4 | 0.2 | 0.4 | 0.3 | 807 |
| SSSLTSLLKTHRMCK | 0.3 | 0.4 | 0.1 | 0.1 | 0.2 | 0.3 | 0.1 | 0.5 | 0.3 | 808 |
| SSLTSLLKTHRMCKY | 0.5 | 0.5 | 0.4 | 0.3 | 0.3 | 0.6 | 0.2 | 0.6 | 0.2 | 809 |
| SLTSLLKTHRMCKYT | 0.3 | 0.3 | 0.1 | 0.2 | 0.2 | 0.4 | 0.2 | 0.3 | 0.2 | 810 |
| LTSLLKTHRMCKYTQ | 0.4 | 0.3 | 0.2 | 0.2 | 0.3 | 0.4 | 0.2 | 0.3 | 0.2 | 811 |
| TSLLKTHRMCKYTQS | 0.4 | 0.3 | 0.2 | 0.2 | 0.2 | 0.4 | 0.2 | 0.3 | 0.2 | 812 |
| SLLKTHRMCKYTQST | 0.3 | 0.2 | 0.1 | 0.2 | 0.3 | 0.4 | 0.2 | 0.2 | 0.1 | 813 |
| LLKTHRMCKYTQSTA | 0.4 | 0.3 | 0.2 | 0.3 | 0.2 | 0.4 | 0.2 | 0.2 | 0.2 | 814 |
| LKTHRMCKYTQSTAL | 0.6 | 0.6 | 0.4 | 0.5 | 0.5 | 0.6 | 0.2 | 0.6 | 0.3 | 815 |
| KTHRMCKYTQSTALQ | 0.4 | 0.4 | 0.2 | 0.4 | 0.2 | 0.4 | 0.2 | 0.2 | 0.2 | 816 |
| THRMCKYTQSTALQE | 0.5 | 0.6 | 0.2 | 0.2 | 0.3 | 0.4 | 0.2 | 0.3 | 0.5 | 817 |
| HRMCKYTQSTALQEL | 0.6 | 0.6 | 0.4 | 1.0 | 0.5 | 0.6 | 1.5 | 0.5 | 0.5 | 818 |
| RMCKYTQSTALQELL | 0.7 | 0.7 | 0.4 | 0.3 | 0.4 | 0.7 | 0.2 | 0.3 | 0.6 | 819 |
| MCKYTQSTALQELLI | 0.6 | 0.6 | 0.5 | 0.3 | 0.7 | 0.8 | 0.2 | 0.4 | 0.7 | 820 |
| CKYTQSTALQELLIQ | 0.6 | 0.6 | 0.5 | 0.4 | 0.3 | 0.4 | 0.3 | 0.3 | 0.4 | 821 |
| KYTQSTALQELLIQQ | 0.8 | 1.0 | 0.7 | 0.7 | 0.8 | 1.0 | 0.5 | 0.8 | 0.9 | 822 |
| YTQSTALQELLIQQW | 0.6 | 0.5 | 0.5 | 0.2 | 0.4 | 0.6 | 0.2 | 0.3 | 0.3 | 823 |
| TQSTALQELLIQQWI | 0.6 | 0.5 | 0.4 | 0.2 | 0.6 | 0.6 | 0.2 | 0.5 | 0.5 | 824 |
| QSTALQELLIQQWIQ | 0.6 | 0.5 | 0.4 | 0.2 | 0.4 | 0.6 | 0.2 | 0.4 | 0.4 | 825 |
| STALQELLIQQWIQF | 0.6 | 0.5 | 0.4 | 0.2 | 0.4 | 0.6 | 0.2 | 0.5 | 0.3 | 826 |
| TALQELLIQQWIQFM | 0.7 | 0.5 | 0.4 | 0.3 | 0.5 | 0.7 | 0.2 | 0.5 | 0.5 | 827 |
| ALQELLIQQWIQFMM | 0.7 | 0.5 | 0.4 | 0.2 | 0.5 | 0.7 | 0.2 | 0.5 | 0.4 | 828 |
| LQELLIQQWIQFMMS | 0.6 | 0.5 | 0.3 | 0.2 | 0.4 | 0.6 | 0.2 | 0.3 | 0.2 | 829 |
| QELLIQQWIQFMMSR | 0.5 | 0.5 | 0.6 | 0.6 | 0.5 | 0.7 | 1.2 | 0.5 | 0.3 | 830 |
| ELLIQQWIQFMMSRR | 0.5 | 0.4 | 0.5 | 0.3 | 0.5 | 0.6 | 0.2 | 0.4 | 0.4 | 831 |
| LLIQQWIQFMMSRRR | 0.5 | 0.5 | 0.7 | 0.3 | 0.5 | 0.7 | 0.2 | 0.4 | 0.3 | 832 |
| LIQQWIQFMMSRRRL | 0.5 | 0.5 | 0.8 | 0.5 | 0.5 | 0.8 | 0.9 | 0.5 | 0.3 | 833 |
| IQQWIQFMMSRRRLL | 0.4 | 0.4 | 0.5 | 0.4 | 0.5 | 0.7 | 0.2 | 0.5 | 0.4 | 834 |
| QQWIQFMMSRRRLLA | 0.6 | 0.5 | 0.6 | 0.4 | 0.6 | 0.8 | 0.3 | 0.4 | 0.5 | 835 |
| QWIQFMMSRRRLLAC | 0.5 | 0.4 | 0.3 | 0.3 | 0.5 | 0.5 | 0.2 | 0.4 | 0.4 | 836 |
| WIQFMMSRRRLLACL | 0.4 | 0.3 | 0.1 | 0.3 | 0.3 | 0.3 | 0.3 | 0.4 | 0.2 | 837 |
| IQFMMSRRRLLACLC | 0.4 | 0.2 | 0.5 | 0.3 | 0.2 | 0.3 | 0.4 | 0.5 | 0.2 | 838 |
| QFMMSRRRLLACLCK | 0.4 | 0.5 | 0.5 | 0.1 | 0.4 | 0.5 | 0.2 | 0.3 | 0.4 | 839 |
| FMMSRRRLLACLCKH | 0.4 | 0.4 | 0.3 | 0.5 | 0.3 | 0.4 | 1.2 | 0.5 | 0.3 | 840 |
| MMSRRRLLACLCKHK | 0.2 | 0.2 | 0.1 | 0.0 | 0.2 | 0.3 | 0.1 | 0.2 | 0.1 | 139 |
| MSRRRLLACLCKHKK | 0.2 | 0.1 | 0.0 | 0.1 | 0.2 | 0.3 | 0.1 | 0.1 | 0.1 | 140 |
| SRRRLLACLCKHKKV | 0.2 | 0.2 | 0.1 | 0.0 | 0.2 | 0.3 | 0.1 | 0.2 | 0.2 | 141 |
| RRRLLACLCKHKKVS | 0.2 | 0.1 | 0.0 | 0.1 | 0.2 | 0.3 | 0.1 | 0.1 | 0.1 | 142 |
| RRLLACLCKHKKVST | 0.2 | 0.1 | 0.1 | 0.2 | 0.2 | 0.4 | 0.2 | 0.2 | 0.2 | 143 |
| RLLACLCKHKKVSTN | 0.3 | 0.2 | 0.3 | 0.3 | 0.3 | 0.5 | 0.2 | 0.4 | 0.3 | 144 |
| LLACLCKHKKVSTNL | 0.4 | 0.4 | 0.4 | 0.4 | 0.5 | 0.7 | 0.2 | 0.4 | 0.3 | 145 |
| LACLCKHKKVSTNLC | 0.3 | 0.3 | 0.2 | 0.2 | 0.3 | 0.4 | 0.2 | 0.3 | 0.2 | 146 |
| ACLCKHKKVSTNLCT | 0.3 | 0.3 | 0.2 | 0.2 | 0.3 | 0.4 | 0.2 | 0.3 | 0.3 | 147 |
| CLCKHKKVSTNLCTH | 0.3 | 0.3 | 0.3 | 0.3 | 0.4 | 0.5 | 0.2 | 0.4 | 0.3 | 148 |
| LCKHKKVSTNLGTHS | 0.3 | 0.4 | 0.3 | 0.3 | 0.3 | 0.4 | 0.3 | 0.5 | 0.3 | 149 |
| CKHKKVSTNLCTHSF | 0.7 | 0.7 | 0.4 | 0.3 | 0.6 | 0.5 | 0.3 | 0.9 | 0.4 | 150 |
| KHKKVSTNLCTHSFR | 0.5 | 0.0 | 0.7 | 0.4 | 0.2 | 0.4 | 0.1 | 0.1 | 0.3 | 151 |
| HKKVSTNLCTHSFRK | 0.6 | 0.4 | 0.8 | 0.6 | 0.5 | 0.7 | 0.3 | 0.6 | 0.3 | 152 |
| KKVSTNLCTHSFRKK | 0.5 | 0.3 | 0.7 | 0.5 | 0.5 | 0.6 | 0.2 | 0.5 | 0.3 | 153 |
| KVSTNLCTHSFRKKQ | 0.4 | 0.3 | 0.5 | 0.5 | 0.4 | 0.6 | 0.2 | 0.5 | 0.3 | 154 |
| VSTNLCTHSFRKKQV | 0.5 | 0.3 | 0.6 | 0.5 | 0.5 | 0.6 | 0.1 | 0.4 | 0.3 | 155 |
| STNLCTHSFRKKQVR | 0.5 | 0.3 | 0.5 | 0.6 | 0.5 | 0.5 | 0.2 | 0.5 | 0.2 | 156 |

TABLE 5
Binding of the sera called SARS-yellow, SARS-green, 1a,
1b, 2, 6, 37, 62 and London to linear peptides of protein E of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | London | green | NO |
| MYSFVSEETGTLIVN | 0.8 | 0.8 | 0.6 | 0.6 | 0.8 | 0.8 | 0.1 | 0.6 | 0.6 | 841 |
| YSFVSEETGTLIVNS | 0.8 | 0.7 | 0.5 | 0.5 | 0.6 | 0.5 | 0.6 | 0.7 | 0.7 | 842 |
| SFVSEETGTLIVNSV | 0.9 | 0.7 | 0.6 | 0.6 | 0.8 | 0.8 | 0.1 | 0.5 | 0.5 | 843 |
| VSEETGTLIVNSVLL | 0.8 | 0.5 | 0.3 | 0.4 | 0.5 | 0.7 | 0.1 | 0.5 | 0.4 | 844 |
| FVSEETGTLIVNSVL | 0.8 | 0.6 | 0.5 | 0.4 | 0.4 | 0.5 | 0.4 | 0.8 | 0.6 | 845 |
| SEETGTLIVNSVLLF | 0.9 | 0.7 | 0.3 | 0.7 | 0.7 | 0.7 | 0.1 | 0.5 | 0.4 | 846 |
| EETGTLIVNSVLLFL | 0.8 | 0.7 | 0.4 | 0.6 | 0.8 | 0.7 | 0.2 | 0.5 | 0.5 | 847 |
| ETGTLIVNSVLLFLA | 0.8 | 0.7 | 0.8 | 0.5 | 0.4 | 0.4 | 0.4 | 0.8 | 0.6 | 848 |
| TGTLIVNSVLLPLAF | 0.7 | 0.5 | 0.3 | 0.6 | 0.7 | 0.6 | 0.2 | 0.4 | 0.4 | 849 |
| GTLIVNSVLLFLAFV | 0.8 | 0.7 | 0.4 | 0.9 | 0.8 | 0.7 | 0.2 | 0.5 | 0.4 | 850 |
| TLIVNSVLLFLAFVV | 0.7 | 0.5 | 0.4 | 0.5 | 0.5 | 0.3 | 0.3 | 0.6 | 0.6 | 851 |
| LIVNSVLLFLAFVVF | 0.8 | 0.6 | 0.4 | 0.7 | 0.6 | 0.6 | 0.3 | 0.4 | 0.4 | 852 |
| IVNSVLLFLAFVVFL | 0.8 | 0.6 | 0.4 | 0.7 | 0.7 | 0.6 | 0.2 | 0.4 | 0.3 | 853 |
| VNSVLLFLAFVVFLL | 0.9 | 0.1 | 1.1 | 0.6 | 0.5 | 0.2 | 0.6 | 1.4 | 0.7 | 854 |
| NSVLLFLAFVVFLLV | 0.8 | 0.6 | 0.6 | 0.9 | 0.7 | 0.7 | 0.2 | 0.6 | 0.3 | 855 |
| SVLLFLAFVVFLLVT | 0.8 | 0.7 | 0.5 | 0.9 | 0.8 | 0.7 | 0.3 | 0.5 | 0.5 | 856 |
| VLLFLAFVVFLLVTL | 0.8 | 0.9 | 0.8 | 0.9 | 0.8 | 0.8 | 0.3 | 0.6 | 0.5 | 857 |
| LLFLAFVVFLLVTLA | 0.8 | 0.6 | 0.5 | 0.7 | 0.7 | 0.7 | 0.2 | 0.5 | 0.4 | 858 |
| LFLAFVVFLLVTLAI | 0.9 | 0.5 | 0.9 | 0.7 | 0.7 | 0.7 | 0.2 | 0.8 | 0.5 | 859 |
| FLAFVVFLLVTLAIL | 0.7 | 0.5 | 0.2 | 0.4 | 0.3 | 0.1 | 0.3 | 0.4 | 0.7 | 860 |
| LAFVVFLLVTLAILT | 0.8 | 0.5 | 0.5 | 0.7 | 0.7 | 0.7 | 0.1 | 0.6 | 0.3 | 861 |
| AFVVFLLVTLAILTA | 0.8 | 0.6 | 0.4 | 0.6 | 0.7 | 0.7 | 0.2 | 0.6 | 0.4 | 862 |
| FVVFLLVTLAILTAL | 0.6 | 0.4 | 0.5 | 0.3 | 0.4 | 0.3 | 0.2 | 0.5 | 0.5 | 863 |
| VVFLLVTLAILTALR | 0.7 | 0.4 | 0.4 | 0.6 | 0.6 | 0.6 | 0.1 | 0.5 | 0.5 | 864 |
| VFLLVTLAILTALRL | 0.7 | 0.4 | 0.4 | 0.5 | 0.6 | 0.6 | 0.0 | 0.5 | 0.4 | 865 |
| FLLVTLAILTALRLC | 0.6 | 0.5 | 0.6 | 0.3 | 0.3 | 0.3 | 0.2 | 0.6 | 0.5 | 866 |
| LLVTLAILTALRLCA | 0.8 | 0.4 | 0.5 | 0.4 | 0.6 | 0.6 | 0.0 | 0.5 | 0.4 | 867 |
| LVTLAILTALRLCAY | 0.8 | 0.5 | 0.6 | 0.7 | 0.7 | 0.7 | 0.1 | 0.5 | 0.3 | 868 |
| VTLAILTALRLCAYC | 0.6 | 0.5 | 0.6 | 0.3 | 0.4 | 0.4 | 0.2 | 0.5 | 0.5 | 869 |
| TLAILTALRLCAYCC | 0.8 | 0.9 | 0.3 | 0.6 | 0.7 | 0.7 | 0.2 | 0.5 | 0.5 | 870 |
| LAILTALRLCAYCCN | 0.8 | 0.7 | 0.5 | 0.5 | 0.8 | 0.8 | 0.2 | 0.5 | 0.5 | 871 |
| AILTALRLCAYCCNI | 0.6 | 0.4 | 0.5 | 0.3 | 0.4 | 0.4 | 0.1 | 0.5 | 0.5 | 872 |
| ILTALRLCAYCCNIV | 0.8 | 0.7 | 0.4 | 0.8 | 0.7 | 0.8 | 0.2 | 0.5 | 0.5 | 873 |
| LTALRLCAYCCNIVN | 0.9 | 0.8 | 0.4 | 0.7 | 0.8 | 0.7 | 0.2 | 0.5 | 0.3 | 874 |
| TALRLCAYCCNIVNV | 0.7 | 0.5 | 0.5 | 0.4 | 0.5 | 0.5 | 0.2 | 0.5 | 0.6 | 875 |
| ALRLCAYCCNIVNVS | 0.9 | 0.8 | 0.5 | 0.7 | 0.8 | 0.7 | 0.2 | 0.5 | 0.5 | 876 |
| LRLCAYCCNIVNVSL | 0.7 | 0.6 | 0.4 | 0.6 | 0.7 | 0.7 | 0.3 | 0.5 | 0.3 | 877 |
| RLCAYCCNIVNVSLV | 0.6 | 0.5 | 0.6 | 0.4 | 0.5 | 0.4 | 0.2 | 0.5 | 0.5 | 878 |
| 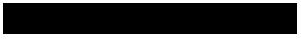 | 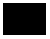 | 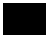 | 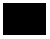 | 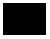 | 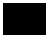 | 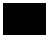 | 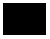 | 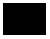 | 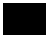 | 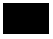 |
| 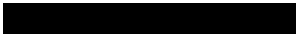 | 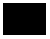 | 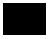 | 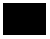 | 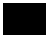 | 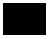 | 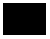 | 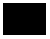 | 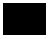 | 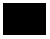 | 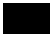 |
| 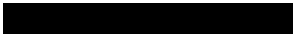 | 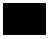 | 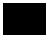 | 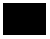 | 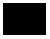 | 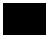 | 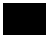 | 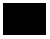 | 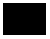 | 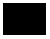 | 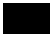 |
| 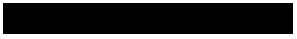 | 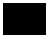 | 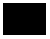 | 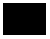 | 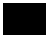 | 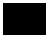 | 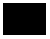 | 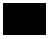 | 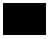 | 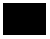 | 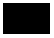 |
| 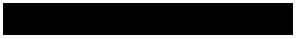 | 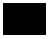 | 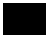 | 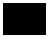 | 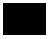 | 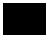 | 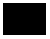 | 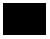 | 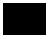 | 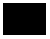 | 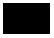 |
| 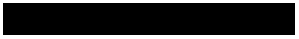 | 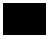 | 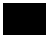 | 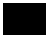 | 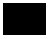 | 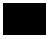 | 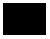 | 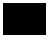 | 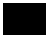 | 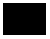 | 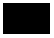 |
| 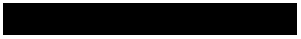 | 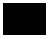 | 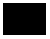 | 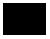 | 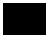 | 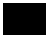 | 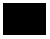 | 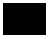 | 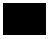 | 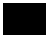 | 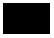 |
| 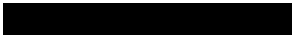 | 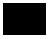 | 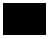 | 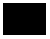 | 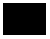 | 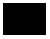 | 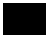 | 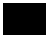 | 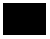 | 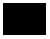 | 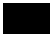 |
| 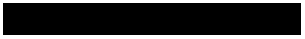 | 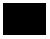 | 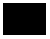 | 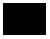 | 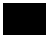 | 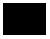 | 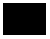 | 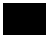 | 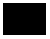 | 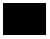 | 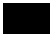 |
| 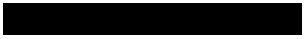 | 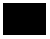 | 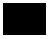 | 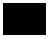 | 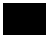 | 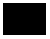 | 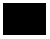 | 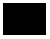 | 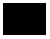 | 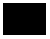 | 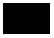 |
| 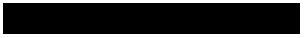 | 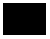 | 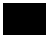 | 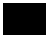 | 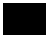 | 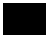 | 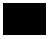 | 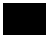 | 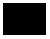 | 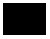 | 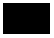 |
| 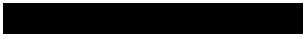 | 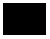 | 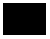 | 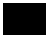 | 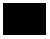 | 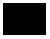 | 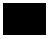 | 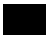 | 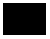 | 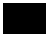 | 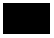 |
| 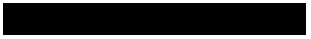 | 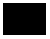 | 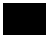 | 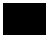 | 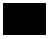 | 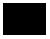 | 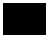 | 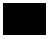 | 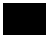 | 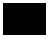 | 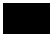 |
| 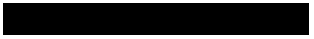 | 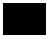 | 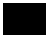 | 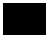 | 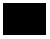 | 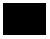 | 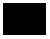 | 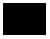 | 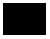 | 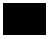 | 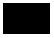 |
| 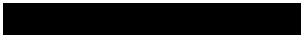 | 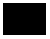 | 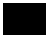 | 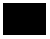 | 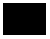 | 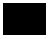 | 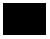 | 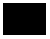 | 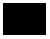 | 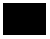 | 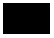 |
| 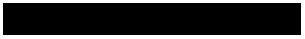 | 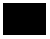 | 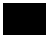 | 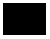 | 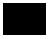 | 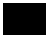 | 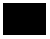 | 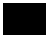 | 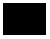 | 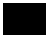 | 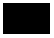 |
| 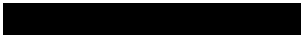 | 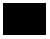 | 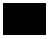 | 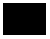 | 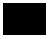 | 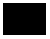 | 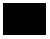 | 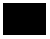 | 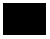 | 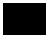 | 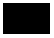 |
| 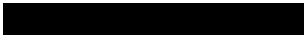 | 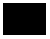 | 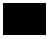 | 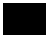 | 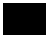 | 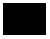 | 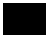 | 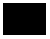 | 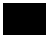 | 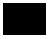 | 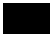 |
| 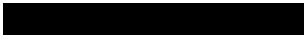 | 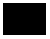 | 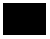 | 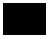 | 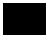 | 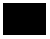 | 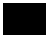 | 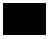 | 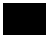 | 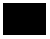 | 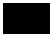 |
| 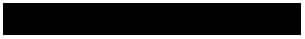 | 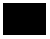 | 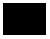 | 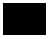 | 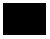 | 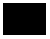 | 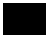 | 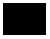 | 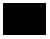 | 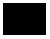 | 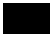 |
| 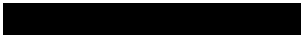 | 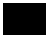 | 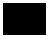 | 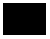 | 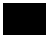 | 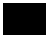 | 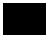 | 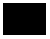 | 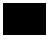 | 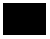 | 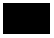 |
| 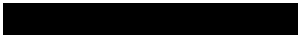 | 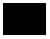 | 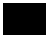 | 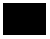 | 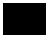 | 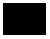 | 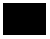 | 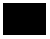 | 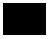 | 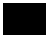 | 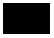 |
| 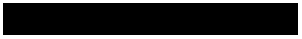 | 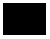 | 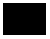 | 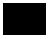 | 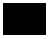 | 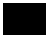 | 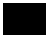 | 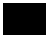 | 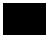 | 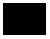 | 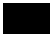 |
| 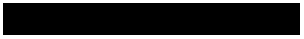 | 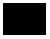 | 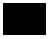 | 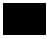 | 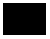 | 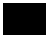 | 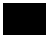 | 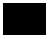 | 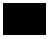 | 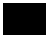 | 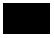 |

TABLE 6
Binding of the sera called SARS-yellow, SARS-green, 1a,
1b, 2, 6, 37, 62 and London to looped/cyclic peptides of protein E
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | London | green | NO |
| MYSFVSEETGTLIVN | 0.7 | 0.5 | 0.6 | 0.5 | 0.7 | 0.8 | 0.8 | 0.4 | 0.4 | 841 |
| YSFVSEETGTLIVNS | 0.7 | 0.5 | 0.7 | 0.4 | 0.8 | 0.8 | 0.7 | 0.4 | 0.4 | 842 |
| SFVSEETGTLIVNSV | 0.5 | 0.4 | 0.5 | 0.4 | 0.6 | 0.7 | 0.7 | 0.5 | 0.4 | 843 |
| VSEETGTLIVNSVLL | 0.7 | 0.6 | 0.6 | 0.5 | 0.7 | 0.9 | 0.7 | 0.5 | 0.6 | 844 |
| FVSEETGTLIVNSVL | 0.6 | 0.6 | 0.4 | 0.5 | 0.6 | 0.8 | 0.8 | 0.5 | 0.6 | 845 |
| SEETGTLIVNSVLLF | 0.4 | 0.3 | 0.3 | 0.5 | 0.4 | 0.7 | 0.5 | 0.5 | 0.4 | 846 |
| EETGTLIVNSVLLFL | 0.4 | 0.3 | 0.3 | 0.4 | 0.4 | 0.7 | 0.5 | 0.4 | 0.3 | 847 |
| ETGTLIVNSVLLFLA | 0.5 | 0.4 | 0.5 | 0.4 | 0.5 | 0.7 | 0.5 | 0.4 | 0.3 | 848 |
| TGTLIVNSVLLFLAF | 0.4 | 0.3 | 0.4 | 0.3 | 0.3 | 0.6 | 0.4 | 0.3 | 0.2 | 849 |
| GTLIVNSVLLFLAFV | 0.2 | 0.0 | 0.6 | 0.3 | 0.2 | 0.4 | 0.0 | 0.4 | 0.4 | 850 |
| TLIVNSVLLFLAFVV | 0.6 | 0.5 | 0.7 | 0.4 | 0.7 | 0.6 | 0.6 | 0.4 | 0.5 | 851 |
| LIVNSVLLFLAFVVF | 0.5 | 0.4 | 0.5 | 0.4 | 0.5 | 0.6 | 0.5 | 0.3 | 0.2 | 852 |
| IVNSVLLFLAFVVFL | 0.6 | 0.4 | 0.5 | 0.3 | 0.6 | 0.6 | 0.7 | 0.3 | 0.2 | 853 |
| VNSVLLFLAFVVFLL | 0.6 | 0.4 | 0.5 | 0.3 | 0.6 | 0.7 | 0.6 | 0.3 | 0.4 | 854 |
| NSVLLFLAFVVFLLV | 0.6 | 0.5 | 0.6 | 0.4 | 0.7 | 0.7 | 0.7 | 0.3 | 0.4 | 855 |
| SVLLFLAFVVFLLVT | 0.6 | 0.5 | 0.5 | 0.4 | 0.6 | 0.6 | 0.6 | 0.4 | 0.6 | 856 |
| VLLFLAFVVFLLVTL | 0.6 | 0.5 | 0.5 | 0.4 | 0.6 | 0.8 | 0.6 | 0.4 | 0.3 | 857 |
| LLFLAFVVFLLVTLA | 0.7 | 0.4 | 0.6 | 0.4 | 0.7 | 0.7 | 0.7 | 0.4 | 0.4 | 858 |
| LFLAFVVFLLVTLAI | 0.5 | 0.5 | 0.6 | 0.5 | 0.6 | 0.7 | 0.9 | 0.5 | 0.7 | 859 |
| FLAFVVFLLVTLAIL | 0.5 | 0.5 | 0.6 | 0.5 | 0.6 | 0.7 | 0.5 | 0.4 | 0.4 | 860 |
| LAFVVFLLVTLAILT | 0.5 | 0.5 | 0.5 | 0.5 | 0.6 | 0.6 | 0.4 | 0.5 | 0.5 | 861 |
| AFVVFLLVTLAILTA | 0.5 | 0.4 | 0.4 | 0.5 | 0.5 | 0.6 | 0.5 | 0.4 | 0.5 | 862 |
| FVVFLLVTLAILTAL | 0.4 | 0.4 | 0.4 | 0.6 | 0.5 | 0.6 | 0.6 | 0.4 | 0.4 | 863 |
| VVFLLVTLAILTALR | 0.5 | 0.3 | 0.5 | 0.5 | 0.4 | 0.7 | 0.5 | 0.5 | 0.2 | 864 |
| VFLLVTLAILTALRL | 0.4 | 0.2 | 0.4 | 0.2 | 0.4 | 0.5 | 0.5 | 0.3 | 0.2 | 865 |
| FLLVTLAILTALRLC | 0.3 | 0.0 | 0.5 | 1 | 0.6 | 0.6 | 0.5 | 1.2 | 0.3 | 866 |
| LLVTLAILTALRLCA | 0.7 | 0.1 | 0.6 | 0.4 | 0.3 | 0.8 | 0.3 | 0.4 | 0.6 | 867 |
| LVTLAILTALRLCAY | 0.6 | 0.5 | 1.0 | 0.4 | 0.7 | 0.6 | 0.4 | 0.4 | 0.6 | 868 |
| VTLAILTALRLCAYC | 0.7 | 0.5 | 0.9 | 0.6 | 0.6 | 0.8 | 0.7 | 0.5 | 0.5 | 869 |
| TLAILTALRLCAYCC | 0.7 | 0.6 | 0.7 | 0.7 | 0.7 | 0.7 | 0.9 | 0.5 | 0.5 | 870 |
| LAILTALRLCAYCCN | 0.8 | 0.4 | 0.6 | 0.5 | 0.6 | 0.7 | 0.8 | 0.4 | 0.5 | 871 |
| AILTALRLCAYCCNI | 0.8 | 0.6 | 0.7 | 0.5 | 0.7 | 0.8 | 0.9 | 0.4 | 0.5 | 872 |
| ILTALRLCAYCCNIV | 0.9 | 0.6 | 1.0 | 0.7 | 0.7 | 0.8 | 0.6 | 0.6 | 0.6 | 873 |
| LTALRLCAYCCNIVN | 0.6 | 0.4 | 0.7 | 0.5 | 0.7 | 0.7 | 0.4 | 0.3 | 0.3 | 874 |
| TALRLCAYCCNIVNV | 0.7 | 0.4 | 0.8 | 0.6 | 0.7 | 0.9 | 0.9 | 0.5 | 0.7 | 875 |
| ALRLCAYCCNIVNVS | 0.8 | 0.8 | 0.7 | 0.7 | 0.7 | 0.8 | 0.7 | 0.6 | 0.6 | 876 |
| LRLCAYCCNIVNVSL | 0.7 | 0.4 | 0.5 | 0.4 | 0.6 | 0.6 | 0.6 | 0.4 | 0.6 | 877 |
| RLCAYCCNIVNVSLV | 0.7 | 0.7 | 0.7 | 0.7 | 0.6 | 0.8 | 0.6 | 0.5 | 0.8 | 878 |
| 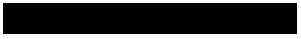 | 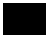 | 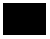 | 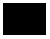 | 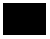 | 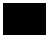 | 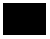 | 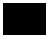 | 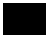 | 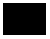 | 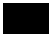 |
| 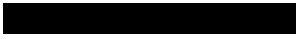 | 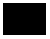 | 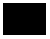 | 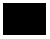 | 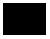 | 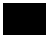 | 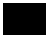 | 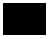 | 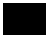 | 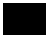 | 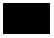 |
| 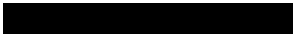 | 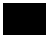 | 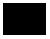 | 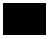 | 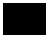 | 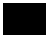 | 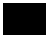 | 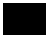 | 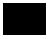 | 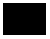 | 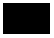 |
| 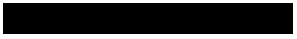 | 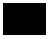 | 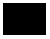 | 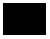 | 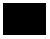 | 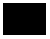 | 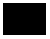 | 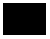 | 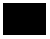 | 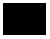 | 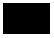 |
| 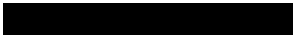 | 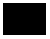 | 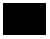 | 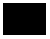 | 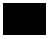 | 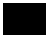 | 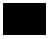 | 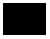 | 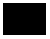 | 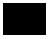 | 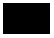 |
| 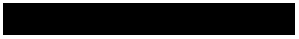 | 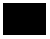 | 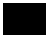 | 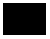 | 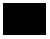 | 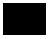 | 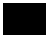 | 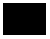 | 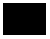 | 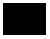 | 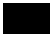 |
| 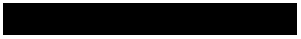 | 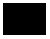 | 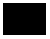 | 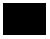 | 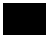 | 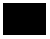 | 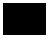 | 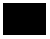 | 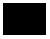 | 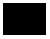 | 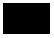 |
| 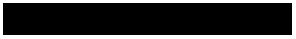 | 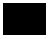 | 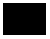 | 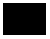 | 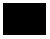 | 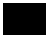 | 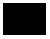 | 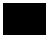 | 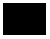 | 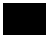 | 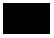 |
| 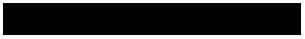 | 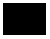 | 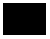 | 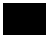 | 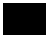 | 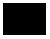 | 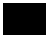 | 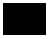 | 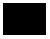 | 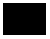 | 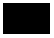 |
| 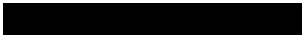 | 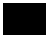 | 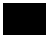 | 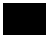 | 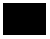 | 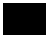 | 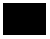 | 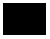 | 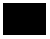 | 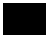 | 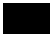 |
| 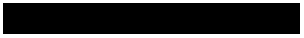 | 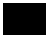 | 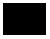 | 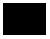 | 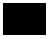 | 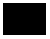 | 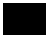 | 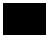 | 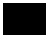 | 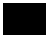 | 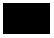 |
| 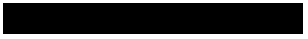 | 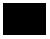 | 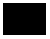 | 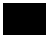 | 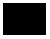 | 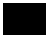 | 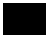 | 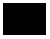 | 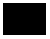 | 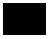 | 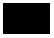 |
| 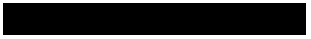 | 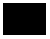 | 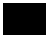 | 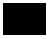 | 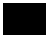 | 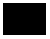 | 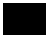 | 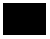 | 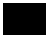 | 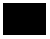 | 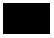 |
| 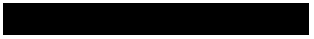 | 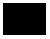 | 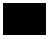 | 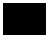 | 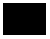 | 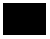 | 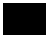 | 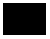 | 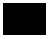 | 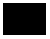 | 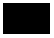 |
| 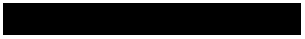 | 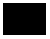 | 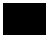 | 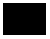 | 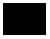 | 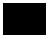 | 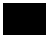 | 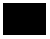 | 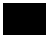 | 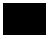 | 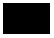 |
| 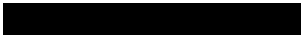 | 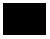 | 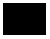 | 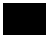 | 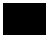 | 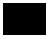 | 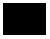 | 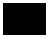 | 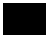 | 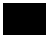 | 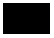 |
| 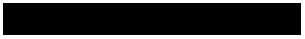 | 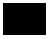 | 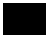 | 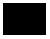 | 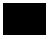 | 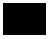 | 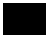 | 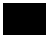 | 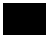 | 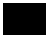 | 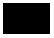 |
| 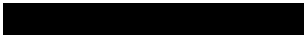 | 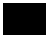 | 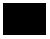 | 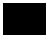 | 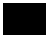 | 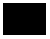 | 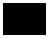 | 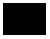 | 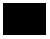 | 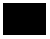 | 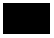 |
| 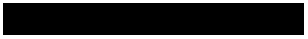 | 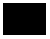 | 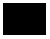 | 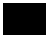 | 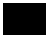 | 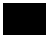 | 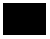 | 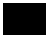 | 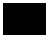 | 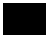 | 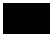 |
| 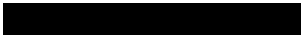 | 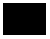 | 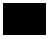 | 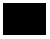 | 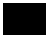 | 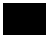 | 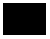 | 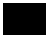 | 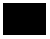 | 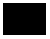 | 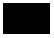 |
| 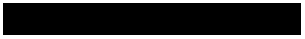 | 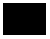 | 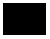 | 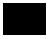 | 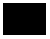 | 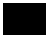 | 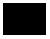 | 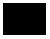 | 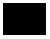 | 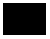 | 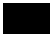 |
| 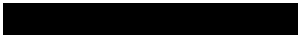 | 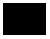 | 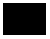 | 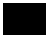 | 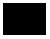 | 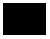 | 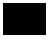 | 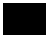 | 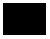 | 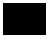 | 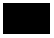 |
| 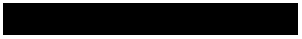 | 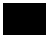 | 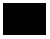 | 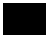 | 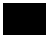 | 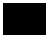 | 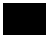 | 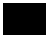 | 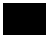 | 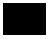 | 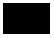 |
| 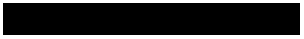 | 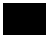 | 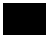 | 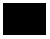 | 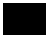 | 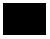 | 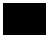 | 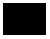 | 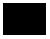 | 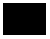 | 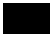 |

TABLE 7
Binding of the sera called SARS-yellow, SARS-green, 1a,
1b, 2, 6, 37, 62 and London to linear peptides of protein M of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | London | green | NO |
| 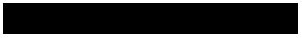 | 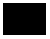 | 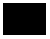 | 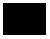 | 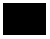 | 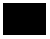 | 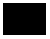 | 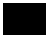 | 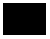 | 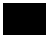 | 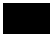 |
| 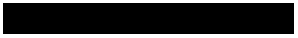 | 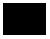 | 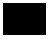 | 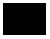 | 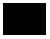 | 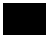 | 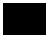 | 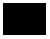 | 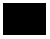 | 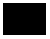 | 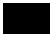 |
| 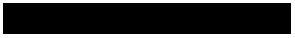 | 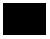 | 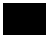 | 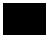 | 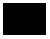 | 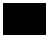 | 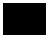 | 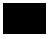 | 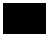 | 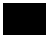 | 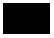 |
| 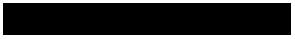 | 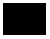 | 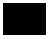 | 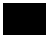 | 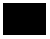 | 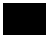 | 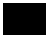 | 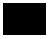 | 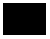 | 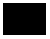 | 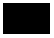 |
| 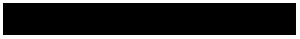 | 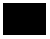 | 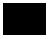 | 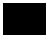 | 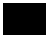 | 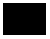 | 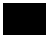 | 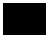 | 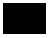 | 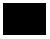 | 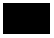 |
| 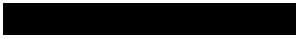 | 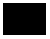 | 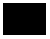 | 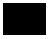 | 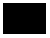 | 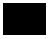 | 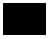 | 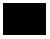 | 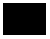 | 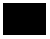 | 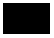 |
| 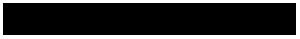 | 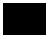 | 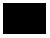 | 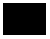 | 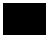 | 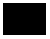 | 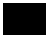 | 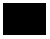 | 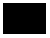 | 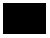 | 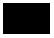 |
| 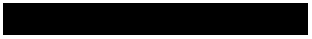 | 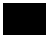 | 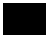 | 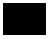 | 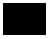 | 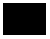 | 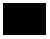 | 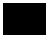 | 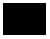 | 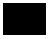 | 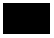 |
| 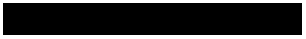 | 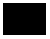 | 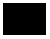 | 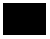 | 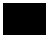 | 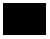 | 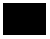 | 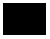 | 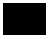 | 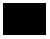 | 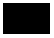 |
| 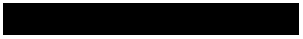 | 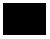 | 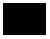 | 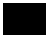 | 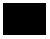 | 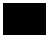 | 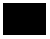 | 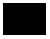 | 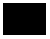 | 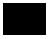 | 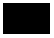 |
| ELKQLLEQWNLVIGF | 0.8 | 0.8 | 0.5 | 0.6 | 0.6 | 0.5 | 0.3 | 0.6 | 0.5 | 879 |
| LKQLLEQWNLVIGFL | 0.7 | 0.5 | 0.6 | 0.7 | 0.7 | 0.7 | 0.1 | 1.0 | 0.2 | 880 |
| KQLLEQWNLVIGFLF | 0.7 | 0.6 | 0.4 | 0.3 | 0.3 | 0.4 | 0.2 | 0.3 | 0.4 | 881 |
| QLLEQWNLVIGFLFL | 0.7 | 0.4 | 0.5 | 0.4 | 0.6 | 0.6 | 0.0 | 0.4 | 0.2 | 882 |
| LLEQWNLVIGFLFLA | 0.7 | 0.6 | 0.4 | 0.2 | 0.4 | 0.4 | 0.2 | 0.3 | 0.3 | 883 |
| LEQWNLVIGFLFLAW | 0.6 | 0.4 | 0.6 | 0.5 | 0.6 | 0.7 | 0.1 | 0.4 | 0.3 | 884 |
| EQWNLVIGFLFLAWI | 0.7 | 0.5 | 0.5 | 0.5 | 0.4 | 0.5 | 0.3 | 0.4 | 0.4 | 885 |
| QWNLVIGFLFLAWIM | 0.7 | 0.4 | 0.6 | 0.4 | 0.7 | 0.8 | 0.1 | 0.3 | 0.2 | 886 |
| WNLVIGFLFLAWIML | 0.7 | 0.6 | 0.4 | 0.3 | 0.4 | 0.4 | 0.2 | 0.3 | 0.4 | 887 |
| NLVIGFLFLAWIMLL | 0.7 | 0.5 | 0.5 | 0.3 | 0.6 | 0.7 | 0.0 | 0.3 | 0.2 | 888 |
| LVIGFLFLAWIMLLQ | 0.7 | 0.5 | 0.5 | 0.4 | 0.4 | 0.4 | 0.1 | 0.3 | 0.4 | 889 |
| VIGFLFLAWIMLLQF | 0.7 | 0.4 | 0.4 | 0.3 | 0.7 | 0.7 | 0.0 | 0.3 | 0.2 | 890 |
| IGFLFLAWIMLLQFA | 0.7 | 0.6 | 0.5 | 0.9 | 0.4 | 0.5 | 0.2 | 0.6 | 0.5 | 891 |
| GFLFLAWIMLLQFAY | 0.7 | 0.5 | 0.5 | 0.3 | 0.7 | 0.7 | 0.0 | 0.3 | 0.2 | 892 |
| FLFLAWIMLLQFAYS | 0.7 | 0.6 | 0.4 | 0.4 | 0.4 | 0.4 | 0.2 | 0.3 | 0.4 | 893 |
| LFLAWIMLLQFAYSN | 0.7 | 0.5 | 0.3 | 0.3 | 0.6 | 0.7 | 0.0 | 0.2 | 0.2 | 894 |
| FLAWIMLLQFAYSNR | 0.7 | 0.6 | 0.5 | 0.4 | 0.4 | 0.4 | 0.2 | 0.4 | 0.5 | 895 |
| LAWIMLLQFAYSNRN | 0.6 | 0.5 | 0.4 | 0.2 | 0.7 | 0.7 | 0.0 | 0.2 | 0.3 | 896 |
| AWIMLLQFAYSNRNR | 0.7 | 0.6 | 0.5 | 0.5 | 0.4 | 0.4 | 0.2 | 0.3 | 0.6 | 897 |
| WIMLLQFAYSNRNRF | 0.6 | 0.5 | 0.5 | 0.3 | 0.7 | 0.7 | 0.1 | 0.3 | 0.3 | 898 |
| IMLLQFAYSNRNRFL | 0.7 | 0.5 | 0.4 | 0.4 | 0.4 | 0.3 | 0.2 | 0.3 | 0.4 | 899 |
| MLLQFAYSNRNRFLY | 0.6 | 0.4 | 0.5 | 0.2 | 0.8 | 0.7 | 0.1 | 0.2 | 0.2 | 900 |
| LLQFAYSNRNRFLYI | 0.7 | 0.6 | 0.4 | 0.3 | 0.3 | 0.3 | 0.2 | 0.3 | 0.5 | 901 |
| LQFAYSNRNRFLYII | 0.7 | 0.5 | 0.6 | 0.3 | 0.7 | 0.7 | 0.0 | 0.2 | 0.2 | 902 |
| QFAYSNRNRFLYIIK | 0.7 | 0.6 | 0.5 | | 0.4 | 0.9 | 0.3 | 0.4 | 0.5 | 191 |
| FAYSNRNRFLYIIKL | 0.6 | 0.4 | 0.7 | 0.3 | 0.7 | 0.7 | 0.1 | 0.3 | 0.2 | 192 |
| AYSNRNRFLYIIKLV | 0.7 | 0.6 | 0.6 | 0.6 | 0.4 | 0.5 | 0.2 | 0.3 | 0.5 | 193 |
| YSNRNRFLYIIKLVF | 0.7 | 0.5 | 0.6 | 0.3 | 0.7 | 0.7 | 0.1 | 0.3 | 0.3 | 194 |
| SNRNRFLYIIKLVFL | 0.6 | 0.5 | 0.4 | 0.5 | 0.4 | 0.4 | 0.3 | 0.3 | 0.4 | 195 |
| NRNRFLYIIKLVFLW | 0.7 | 0.4 | 0.6 | 0.3 | 0.7 | 0.7 | 0.1 | 0.3 | 0.3 | 196 |
| RNRFLYIIKLVFLWL | 0.6 | 0.5 | 0.4 | 0.5 | 0.4 | 0.5 | 0.3 | 0.3 | 0.4 | 197 |
| NRFLYIIKLVFLWLL | 0.7 | 0.5 | 0.5 | 0.3 | 0.7 | 0.7 | 0.1 | 0.3 | 0.3 | 198 |
| RFLYIIKLVFLWLLW | 0.7 | 0.7 | 0.6 | 0.8 | 0.4 | 0.4 | 0.3 | 0.6 | 0.6 | 199 |
| FLYIIKLVFLWLLWP | 0.8 | 0.5 | 1.0 | 0.3 | 0.8 | 0.9 | 0.1 | 0.3 | 0.3 | 200 |
| LYIIKLVFLWLLWPV | 0.8 | 0.6 | 0.6 | 0.4 | 0.4 | 0.4 | 0.2 | 0.3 | 0.5 | 903 |
| YIIKLVFLWLLWPVT | 0.8 | 0.6 | 0.9 | 0.4 | 0.7 | 0.8 | 0.1 | 0.3 | 0.4 | 904 |
| IIKLVFLWLLWPVTL | 0.7 | 0.5 | 0.4 | 0.4 | 0.3 | 0.3 | 0.2 | 0.3 | 0.4 | 905 |
| IKLVFLWLLWPVTLA | 0.7 | 0.5 | 0.7 | 0.5 | 0.6 | 0.7 | 0.1 | 0.6 | 0.3 | 906 |
| KLVFLWLLWPVTLAC | 0.7 | 0.6 | 0.5 | 0.4 | 0.5 | 0.5 | 0.2 | 0.4 | 0.4 | 907 |
| LVFLWLLWPVTLACF | 0.7 | 0.5 | 0.4 | 0.6 | 0.6 | 0.8 | 0.1 | 0.7 | 0.2 | 908 |
| VFLWLLWPVTLACFV | 0.8 | 0.6 | 0.6 | 0.4 | 0.5 | 0.5 | 0.3 | 0.5 | 0.6 | 909 |
| FLWLLWPVTLACFVL | 0.6 | 0.4 | 0.5 | 0.3 | 0.6 | 0.7 | 0.1 | 0.4 | 0.2 | 910 |
| LWLLWPVTLACFVLA | 0.7 | 0.6 | 0.4 | 0.5 | 0.4 | 0.4 | 0.2 | 0.4 | 0.4 | 911 |
| WLLWPVTLACFVLAA | 0.6 | 0.5 | 0.5 | 0.4 | 0.6 | 0.6 | 0.0 | 0.3 | 0.3 | 912 |
| LLWPVTLACFVLAAV | 0.7 | 0.5 | 0.6 | 0.4 | 0.5 | 0.5 | 0.1 | 0.3 | 0.5 | 913 |
| LWPVTLACFVLAAVY | 0.7 | 0.6 | 0.6 | 0.3 | 0.7 | 0.8 | 0.0 | 0.3 | 0.2 | 914 |
| WPVTLACFVLAAVYR | 0.7 | 0.5 | 0.4 | 0.3 | 0.4 | 0.4 | 0.2 | 0.3 | 0.4 | 915 |
| PVTLACFVLAAVYRI | 0.7 | 0.5 | 0.5 | 0.3 | 0.6 | 0.7 | 0.0 | 0.3 | 0.2 | 916 |
| VTLACFVLAAVYRIN | 0.7 | 0.6 | 0.5 | 0.4 | 0.4 | 0.5 | 0.1 | 0.3 | 0.5 | 917 |
| TLACFVLAAVYRINW | 0.7 | 0.5 | 0.5 | 0.3 | 0.8 | 0.8 | 0.0 | 0.3 | 0.3 | 918 |
| LACFVLAAVYRINWV | 0.7 | 0.7 | 0.6 | 0.4 | 0.5 | 0.5 | 0.3 | 0.4 | 0.6 | 919 |
| ACFVLAAVYRINWVT | 0.7 | 0.6 | 0.5 | 0.3 | 0.8 | 0.9 | 0.0 | 0.3 | 0.3 | 920 |
| CFVLAAVYRINWVTG | 0.8 | 0.6 | 0.6 | 0.6 | 0.4 | 0.4 | 0.2 | 0.4 | 0.5 | 921 |
| FVLAAVYRINWVTGG | 0.9 | 0.7 | 0.6 | 0.3 | 0.8 | 0.8 | 0.1 | 0.3 | 0.3 | 922 |
| VLAAVYRINWVTGGI | 0.8 | 0.7 | 0.7 | 0.5 | 0.6 | 0.6 | 0.2 | 0.4 | 0.6 | 923 |
| LAAVYRINWVTGGIA | 0.7 | 0.5 | 0.6 | 0.3 | 0.8 | 0.9 | 0.1 | 0.3 | 0.3 | 924 |
| AAVYRINWVTGGIAI | 0.9 | 0.7 | 0.7 | 0.5 | 0.6 | 0.5 | 0.3 | 0.4 | 0.6 | 925 |
| AVYRINWVTGGIAIA | 0.7 | 0.6 | 0.6 | 0.2 | 0.8 | 0.8 | 0.1 | 0.3 | 0.3 | 926 |
| VYRINWVTGGIAIAM | 0.9 | 0.8 | 0.8 | 1.0 | 0.6 | 0.6 | 0.3 | 0.6 | 0.7 | 927 |
| YRINWVTGGIAIAMA | 0.7 | 0.6 | 0.7 | 0.3 | 0.8 | 0.8 | 0.1 | 0.3 | 0.3 | 928 |
| RINWVTGGIAIAMAC | 0.7 | 0.7 | 0.6 | 0.8 | 0.5 | 0.5 | 0.3 | 0.5 | 0.6 | 929 |
| 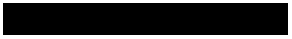 | 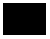 | 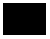 | 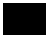 | 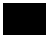 | 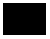 | 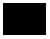 | 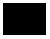 | 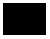 | 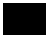 | 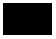 |
| 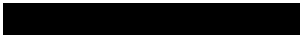 | 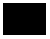 | 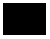 | 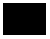 | 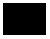 | 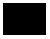 | 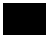 | 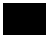 | 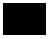 | 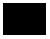 | 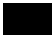 |
| 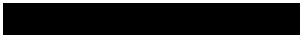 | 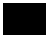 | 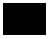 | 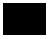 | 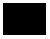 | 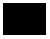 | 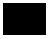 | 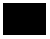 | 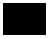 | 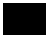 | 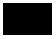 |
| 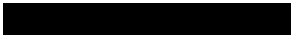 | 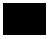 | 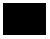 | 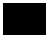 | 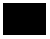 | 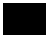 | 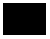 | 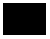 | 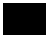 | 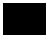 | 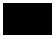 |
| 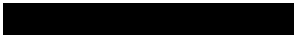 | 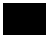 | 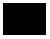 | 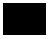 | 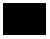 | 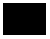 | 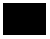 | 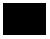 | 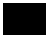 | 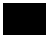 | 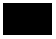 |
| 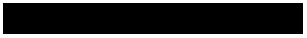 | 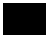 | 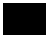 | 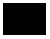 | 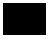 | 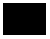 | 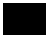 | 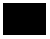 | 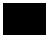 | 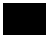 | 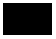 |
| 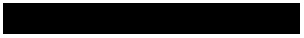 | 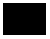 | 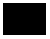 | 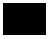 | 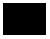 | 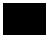 | 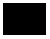 | 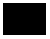 | 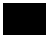 | 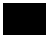 | 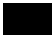 |
| 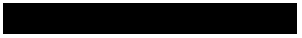 | 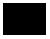 | 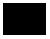 | 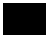 | 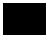 | 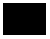 | 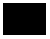 | 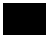 | 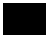 | 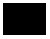 | 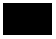 |
| AIAMACIVGLMWLSY | 0.7 | 0.5 | 0.6 | 0.4 | 0.7 | 0.7 | 0.1 | 0.4 | 0.3 | 930 |
| IAMACIVGLMWLSYF | 0.6 | 0.5 | 0.4 | 0.3 | 0.3 | 0.3 | 0.2 | 0.3 | 0.3 | 931 |
| AMACIVGLMWLSYFV | 0.7 | 0.5 | 0.6 | 0.4 | 0.6 | 0.6 | 0.1 | 0.4 | 0.2 | 932 |
| MACIVGLMWLSYFVA | 0.7 | 0.5 | 0.4 | 0.3 | 0.4 | 0.4 | 0.2 | 0.3 | 0.3 | 933 |
| ACIVGLMWLSYFVAS | 0.7 | 0.5 | 0.5 | 0.4 | 0.6 | 0.7 | 0.1 | 0.3 | 0.2 | 934 |
| CIVGLMWLSYFVASF | 0.7 | 0.5 | 0.3 | 0.3 | 0.4 | 0.4 | 0.1 | 0.3 | 0.3 | 935 |
| IVGLMWLSYFVASFR | 0.6 | 0.4 | 0.5 | 0.3 | 0.5 | 0.7 | 0.1 | 0.3 | 0.2 | 936 |
| VGLMWLSYFVASFRL | 0.6 | 0.5 | 0.4 | 0.5 | 0.4 | 0.4 | 0.1 | 0.5 | 0.4 | 937 |
| GLMWLSYFVASFRLF | 0.7 | 0.4 | 0.4 | 0.6 | 0.5 | 0.6 | 0.1 | 0.4 | 0.3 | 938 |
| LMWLSYFVASFRLFA | 0.6 | 0.4 | 0.4 | 0.3 | 0.3 | 0.4 | 0.1 | 0.3 | 0.5 | 209 |
| MWLSYFVASFRLFAR | 0.6 | 0.3 | 0.5 | 0.3 | 0.6 | 0.7 | 0.1 | 0.3 | 0.2 | 210 |
| WLSYFVASFRLFART | 0.6 | 0.5 | 0.4 | 0.4 | 0.4 | 0.4 | 0.1 | 0.3 | 0.4 | 211 |
| LSYFVASFRLFARTR | 0.7 | 0.4 | 0.4 | 0.4 | 0.6 | 0.7 | 0.1 | 0.3 | 0.2 | 212 |
| SYFVASFRLFARTRS | 0.7 | 0.6 | 0.5 | 0.5 | 0.4 | 0.4 | 0.1 | 0.3 | 0.4 | 213 |
| YFVASFRLFARTRSM | 0.6 | 0.4 | 0.4 | 0.4 | 0.4 | 0.6 | 0.1 | 0.3 | 0.3 | 214 |
| FVASFRLFARTRSMW | 0.9 | 1.1 | 0.7 | 1.3 | 0.6 | 0.6 | 0.3 | 0.9 | 0.7 | 215 |
| VASFRLEARTRSMWS | 0.7 | 0.5 | 0.5 | 0.3 | 0.7 | 0.8 | 0.0 | 0.3 | 0.3 | 216 |
| ASFRLFARTRSMWSF | 0.7 | 0.6 | 0.5 | 0.7 | 0.4 | 0.4 | 0.2 | 0.3 | 0.4 | 939 |
| SFRLFARTRSMWSFN | 0.8 | 0.7 | 0.5 | 0.2 | 0.8 | 0.7 | 0.1 | 0.3 | 0.4 | 940 |
| FRLFARTRSMWSFNP | 0.8 | 0.5 | 0.6 | 0.5 | 0.5 | 0.5 | 0.2 | 0.4 | 0.6 | 941 |
| RLFARTRSMWSFNPE | 0.9 | 0.9 | 0.6 | 0.4 | 1.0 | 0.9 | 0.1 | 0.4 | 0.5 | 942 |
| LFARTRSMWSFNPET | 0.8 | 0.7 | 0.5 | 0.6 | 0.5 | 0.5 | 0.2 | 0.4 | 0.5 | 943 |
| FARTRSMWSFNPETN | 0.8 | 0.7 | 0.7 | 0.4 | 0.9 | 0.8 | 0.1 | 0.4 | 0.3 | 944 |
| ARTRSMWSFNPETNI | 0.9 | 0.8 | 0.6 | 0.7 | 0.6 | 0.5 | 0.3 | 0.4 | 0.7 | 945 |
| RTRSMWSFNPETNIL | 0.9 | 0.8 | 0.8 | 0.4 | 0.9 | 0.9 | 0.1 | 0.3 | 0.3 | 946 |
| TRSMWSFNPETNILL | 0.8 | 0.7 | 0.5 | 0.5 | 0.5 | 0.4 | 0.3 | 0.3 | 0.5 | 947 |
| RSMWSFNPETNILLN | 0.8 | 0.6 | 0.6 | 0.3 | 0.8 | 0.7 | 0.1 | 0.3 | 0.2 | 948 |
| SMWSFNPETNILLNV | 0.8 | 0.7 | 0.5 | 0.5 | 0.5 | 0.5 | 0.2 | 0.4 | 0.5 | 949 |
| MWSFNPETNILLNVP | 0.8 | 0.5 | 0.8 | 0.4 | 0.9 | 0.9 | 0.1 | 0.3 | 0.3 | 950 |
| WSFNPETNILLNVPL | 1.5 | 1.7 | 1.3 | 1.0 | 0.9 | 1.3 | 1.1 | 1.0 | 1.5 | 951 |
| SFNPETNILLNVPLR | 0.7 | 0.4 | 0.5 | 0.3 | 0.7 | 0.7 | 0.1 | 0.3 | 0.3 | 952 |
| PNPETNILLNVPLRG | 0.8 | 0.6 | 0.5 | 0.5 | 0.5 | 0.4 | 0.2 | 0.3 | 0.5 | 953 |
| NPETNILLNVPLRGT | 0.8 | 0.6 | 0.8 | 0.3 | 0.9 | 0.9 | 0.1 | 0.3 | 0.3 | 954 |
| PETNILLNVPLRGTI | 0.8 | 0.6 | 0.7 | 0.4 | 0.9 | 0.5 | 0.3 | 0.4 | 0.5 | 955 |
| ETNILLNVPLRGTIV | 0.7 | 0.5 | 0.5 | 0.4 | 0.6 | 0.7 | 0.1 | 0.4 | 0.3 | 956 |
| TNILLNVPLRGTIVT | 0.6 | 0.5 | 0.6 | 0.4 | 0.4 | 0.5 | 0.2 | 0.3 | 0.5 | 957 |
| NILLNVPLRGTIVTR | 0.7 | 0.5 | 0.8 | 0.4 | 0.6 | 0.7 | 0.1 | 0.4 | 0.3 | 217 |
| ILLNVPLRGTIVTRP | 0.6 | 0.4 | 0.4 | 0.2 | 0.4 | 0.4 | 0.2 | 0.3 | 0.4 | 218 |
| LLNVPLRGTIVTRPL | 0.8 | 0.5 | 0.5 | 0.5 | 0.6 | 0.7 | 0.1 | 0.4 | 0.3 | 219 |
| LNVPLRGTIVTRPLM | 0.7 | 0.5 | 0.6 | 0.5 | 0.5 | 0.5 | 0.1 | 0.4 | 0.4 | 220 |
| NVPLRGTIVTRPLME | 0.8 | 0.6 | 0.4 | 0.3 | 0.7 | 0.8 | 0.1 | 0.3 | 0.4 | 221 |
| VPLRGTIVTRPLMES | 0.7 | 0.6 | 0.7 | 0.4 | 0.6 | 0.5 | 0.4 | 0.4 | 0.5 | 222 |
| PLRGTIVTRPLMESE | 0.8 | 0.7 | 0.4 | 0.4 | 0.8 | 0.7 | 0.0 | 0.3 | 0.5 | 223 |
| LRGTIVTRPLMESEL | 0.7 | 0.5 | 0.3 | 0.3 | 0.5 | 0.4 | 1.0 | 0.3 | 0.4 | 224 |
| RGTIVTRPLMESELV | 0.9 | 0.6 | 0.6 | 0.4 | 0.9 | 0.9 | 0.1 | 0.3 | 0.3 | 225 |
| GTIVTRPLMESELVI | 0.8 | 0.7 | 0.7 | 0.5 | 0.6 | 0.7 | 0.2 | 0.3 | 0.7 | 226 |
| TIVTRPLMESELVIG | 0.8 | 0.6 | 0.6 | 0.4 | 0.7 | 0.9 | 0.1 | 0.3 | 0.3 | 227 |
| IVTRPLMESELVIGA | 0.8 | 0.7 | 0.6 | 0.4 | 0.6 | 0.6 | 0.5 | 0.3 | 0.6 | 229 |
| VTRPLMESELVIGAV | 0.8 | 0.6 | 0.9 | 0.2 | 1.0 | 0.9 | 0.1 | 0.3 | 0.3 | 230 |
| TRPLMESELVIGAVI | 0.8 | 0.7 | 0.7 | 0.6 | 0.7 | 0.6 | 0.2 | 0.3 | 0.8 | 231 |
| RPLMESELVIGAVII | 0.8 | 0.6 | 0.7 | 0.3 | 0.6 | 0.9 | 0.1 | 0.4 | 0.3 | 232 |
| PLMESELVIGAVIIR | 0.7 | 0.6 | 0.7 | 0.4 | 0.7 | 0.5 | 0.2 | 0.3 | 0.5 | 958 |
| LMESELVIGAVIIRG | 0.7 | 0.5 | 0.7 | 0.3 | 0.9 | 0.8 | 0.1 | 0.3 | 0.3 | 959 |
| MESELVIGAVIIRGH | 0.8 | 0.7 | 0.8 | 0.6 | 0.5 | 0.5 | 0.2 | 0.4 | 0.7 | 960 |
| ESELVIGAVIIRGHL | 0.8 | 0.6 | 0.8 | 0.3 | 0.9 | 0.8 | 0.1 | 0.3 | 0.3 | 961 |
| SELVIGAVIIRGHLR | 0.7 | 0.6 | 0.6 | 0.7 | 0.5 | 0.4 | 0.3 | 0.4 | 0.5 | 962 |
| ELVIGAVIIRGHLRM | 0.8 | 0.6 | 0.7 | 0.3 | 0.8 | 0.8 | 0.1 | 0.3 | 0.3 | 963 |
| LVIGAVIIRGHLRMA | 0.7 | 0.5 | 0.5 | 0.6 | 0.4 | 0.4 | 0.3 | 0.3 | 0.4 | 964 |
| 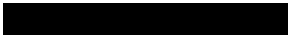 | 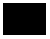 | 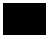 | 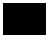 | 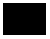 | 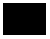 | 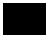 | 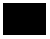 | 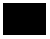 | 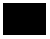 | 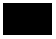 |
| 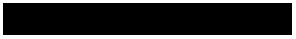 | 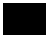 | 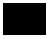 | 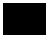 | 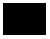 | 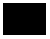 | 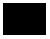 | 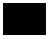 | 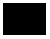 | 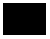 | 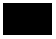 |
| 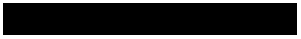 | 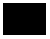 | 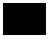 | 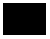 | 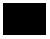 | 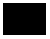 | 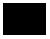 | 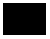 | 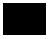 | 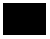 | 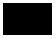 |
| 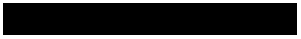 | 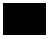 | 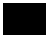 | 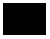 | 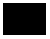 | 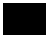 | 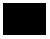 | 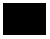 | 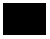 | 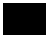 | 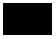 |
| 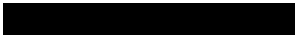 | 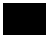 | 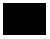 | 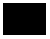 | 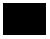 | 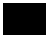 | 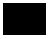 | 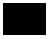 | 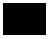 | 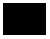 | 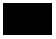 |
| 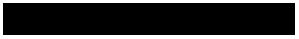 | 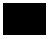 | 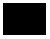 | 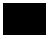 | 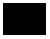 | 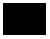 | 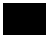 | 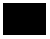 | 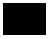 | 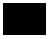 | 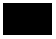 |
| 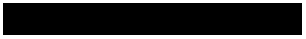 | 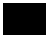 | 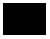 | 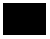 | 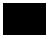 | 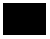 | 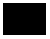 | 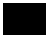 | 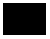 | 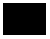 | 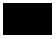 |
| 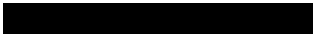 | 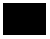 | 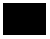 | 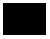 | 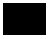 | 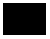 | 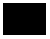 | 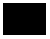 | 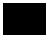 | 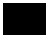 | 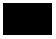 |
| 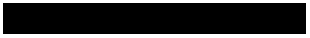 | 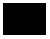 | 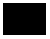 | 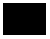 | 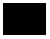 | 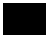 | 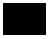 | 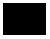 | 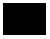 | 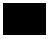 | 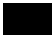 |
| 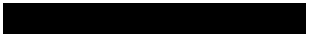 | 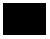 | 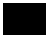 | 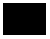 | 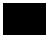 | 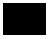 | 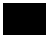 | 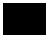 | 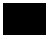 | 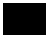 | 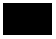 |
| 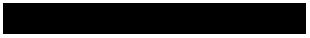 | 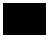 | 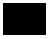 | 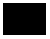 | 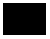 | 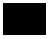 | 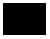 | 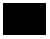 | 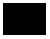 | 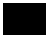 | 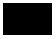 |
| 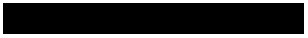 | 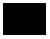 | 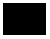 | 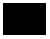 | 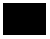 | 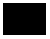 | 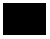 | 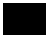 | 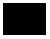 | 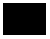 | 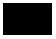 |
| 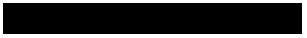 | 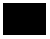 | 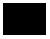 | 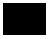 | 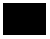 | 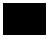 | 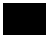 | 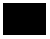 | 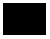 | 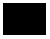 | 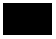 |
| 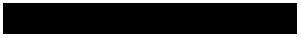 | 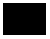 | 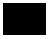 | 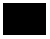 | 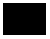 | 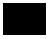 | 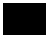 | 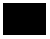 | 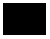 | 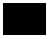 | 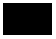 |
| 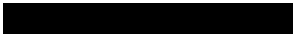 | 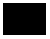 | 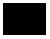 | 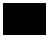 | 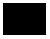 | 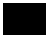 | 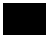 | 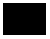 | 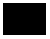 | 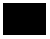 | 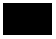 |
| 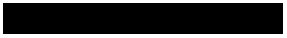 | 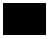 | 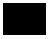 | 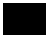 | 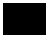 | 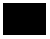 | 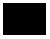 | 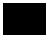 | 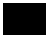 | 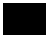 | 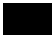 |
| 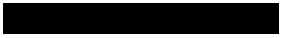 | 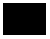 | 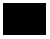 | 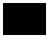 | 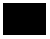 | 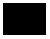 | 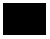 | 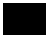 | 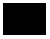 | 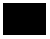 | 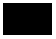 |
| 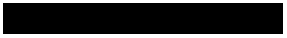 | 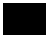 | 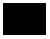 | 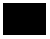 | 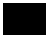 | 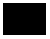 | 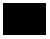 | 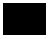 | 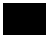 | 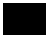 | 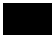 |
| 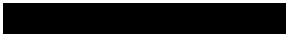 | 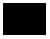 | 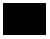 | 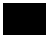 | 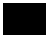 | 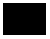 | 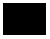 | 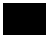 | 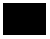 | 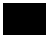 | 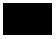 |
| RCDIKDLPKEITVAT | 0.7 | 0.5 | 0.5 | 0.3 | 0.5 | 0.6 | 0.2 | 0.3 | 0.5 | 965 |
| CDIKDLPKEITVATS | 0.6 | 0.3 | 0.3 | 0.3 | 0.5 | 0.7 | 0.0 | 0.3 | 0.2 | 966 |
| DIKDLPKEITVATSR | 0.7 | 0.5 | 0.5 | 0.5 | 0.6 | 0.5 | 0.2 | 0.4 | 0.5 | 967 |
| IKDLPKEITVATSRT | 0.7 | 0.5 | 0.6 | 0.3 | 0.8 | 0.8 | 0.0 | 0.3 | 0.2 | 968 |
| KDLPKEITVATSRTL | 0.7 | 0.6 | 0.6 | 0.5 | 0.5 | 0.5 | 0.2 | 0.3 | 0.5 | 969 |
| DLPKEITVATSRTLS | 0.7 | 0.2 | 0.7 | 0.2 | 0.5 | 0.7 | 0.0 | 0.3 | 0.3 | 970 |
| LPKEITVATSRTLSY | 0.7 | 0.5 | 0.6 | 0.4 | 0.4 | 0.5 | 0.2 | 0.3 | 0.4 | 971 |
| PKEITVATSRTLSYY | 0.6 | 0.4 | 0.5 | 0.2 | 0.8 | 0.7 | 0.1 | 0.3 | 0.1 | 972 |
| KEITVATSRTLSYYK | 0.7 | 0.6 | 0.6 | 0.0 | 0.5 | 0.6 | 0.2 | 0.3 | 0.4 | 973 |
| EITVATSRTLSYYKL | 0.7 | 0.5 | 0.6 | 0.4 | 0.8 | 0.9 | 0.1 | 0.3 | 0.4 | 974 |
| ITVATSRTLSYYKLG | 0.6 | 0.4 | 0.5 | 0.5 | 0.4 | 0.4 | 0.2 | 0.3 | 0.5 | 975 |
| TVATSRTLSYYKLGA | 0.7 | 0.6 | 0.7 | 0.6 | 0.8 | 0.8 | 0.1 | 0.4 | 0.3 | 976 |
| VATSRTLSYYKLGAS | 0.6 | 0.5 | 0.6 | 0.8 | 0.5 | 0.5 | 0.3 | 0.3 | 0.4 | 977 |
| ATSRTLSYYKLGASQ | 0.7 | 0.5 | 0.7 | 0.3 | 0.8 | 0.7 | 0.1 | 0.3 | 0.2 | 978 |
| TSRTLSYYKLGASQR | 0.7 | 0.7 | 0.7 | 0.9 | 0.7 | 0.5 | 0.5 | 0.5 | 0.7 | 979 |
| SRTLSYYKLGASQRV | 0.8 | 0.5 | 0.8 | 0.4 | 0.8 | 0.8 | 0.1 | 0.3 | 0.2 | 980 |
| RTLSYYKLGASQRVG | 0.7 | 0.6 | 0.7 | 0.8 | 0.5 | 0.5 | 0.4 | 0.3 | 0.5 | 981 |
| 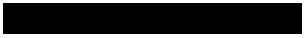 | 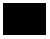 | 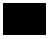 | 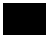 | 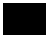 | 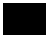 | 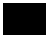 | 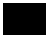 | 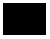 | 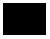 | 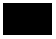 |
| 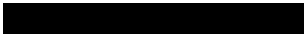 | 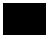 | 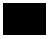 | 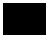 | 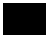 | 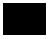 | 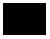 | 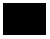 | 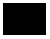 | 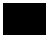 | 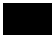 |
| 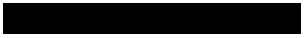 | 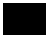 | 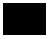 | 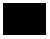 | 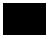 | 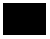 | 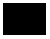 | 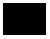 | 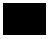 | 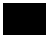 | 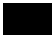 |
| 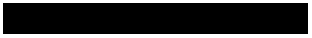 | 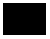 | 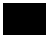 | 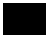 | 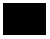 | 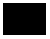 | 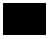 | 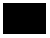 | 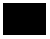 | 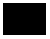 | 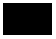 |
| 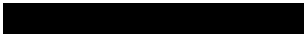 | 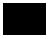 | 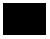 | 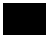 | 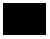 | 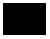 | 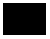 | 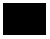 | 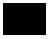 | 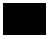 | 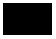 |
| 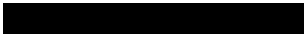 | 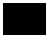 | 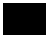 | 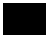 | 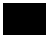 | 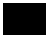 | 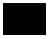 | 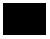 | 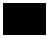 | 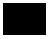 | 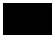 |
| 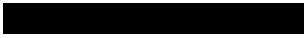 | 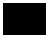 | 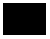 | 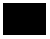 | 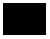 | 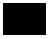 | 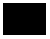 | 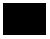 | 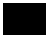 | 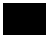 | 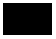 |
| 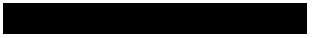 | 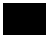 | 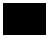 | 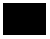 | 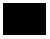 | 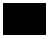 | 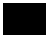 | 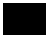 | 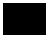 | 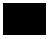 | 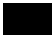 |
| 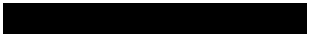 | 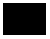 | 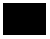 | 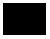 | 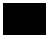 | 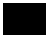 | 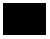 | 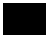 | 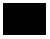 | 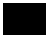 | 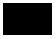 |
| SQRVGTDSGFAAYNR | 0.8 | 0.6 | 0.4 | 0.6 | 0.5 | 0.4 | 0.5 | 0.4 | 0.5 | 982 |
| QRVGTDSGFAAYNRY | 0.7 | 0.5 | 0.5 | 0.3 | 0.7 | 0.7 | 0.1 | 0.3 | 0.3 | 983 |
| RVGTDSGFAAYNRYR | 0.7 | 0.1 | 0.5 | 0.5 | 0.6 | 0.2 | 0.3 | 0.3 | 0.5 | 984 |
| VGTDSGFAAYNRYRI | 0.8 | 0.4 | 0.4 | 0.3 | 0.6 | 0.7 | 0.1 | 0.3 | 0.3 | 985 |
| GTDSGFAAYNRYRIG | 0.6 | 0.4 | 0.4 | 0.7 | 0.6 | 0.5 | 0.2 | 0.3 | 0.4 | 986 |
| TDSGFAAYNRYRIGN | 0.6 | 0.4 | 0.5 | 0.4 | 0.4 | 0.6 | 0.0 | 0.3 | 0.3 | 987 |
| DSGFAAYNRYRIGNY | 0.6 | 0.4 | 0.4 | 0.5 | 0.4 | 0.4 | 0.1 | 0.3 | 0.3 | 988 |
| SGFAAYNRYRIGNYK | 0.8 | 0.4 | 0.6 | 0.8 | 0.7 | 0.8 | 0.0 | 0.4 | 0.3 | 989 |
| GFAAYNRYRIGNYKL | 0.6 | 0.4 | 0.4 | 0.8 | 0.5 | 0.5 | 0.1 | 0.4 | 0.3 | 990 |
| FAAYNRYRIGNYKLN | 0.6 | 0.3 | 0.5 | 0.4 | 0.5 | 0.7 | 0.0 | 0.3 | 0.2 | 991 |
| AAYNRYRIGNYKLNT | 0.7 | 0.5 | 0.7 | 0.8 | 0.6 | 0.5 | 0.2 | 0.4 | 0.5 | 992 |
| AYNRYRIGNYKLNTD | 0.7 | 0.2 | 0.3 | 0.4 | 0.5 | 0.7 | 0.0 | 0.5 | 0.2 | 993 |
| YNRYRIGNYKLNTDH | 0.7 | 0.6 | 0.4 | 0.5 | 0.5 | 0.4 | 0.1 | 0.5 | 0.4 | 994 |
| NRYRIGNYKLNTDHA | 0.8 | 0.6 | 0.5 | 0.3 | 0.5 | 0.6 | 0.1 | 0.2 | 0.2 | 995 |
| RYRIGNYKLNTDHAG | 0.7 | 0.5 | 0.4 | 0.2 | 0.6 | 0.5 | 0.2 | 0.5 | 0.4 | 996 |
| YRIGNYKLNTDHAGS | 0.7 | 0.6 | 0.5 | 0.2 | 0.8 | 0.7 | 0.1 | 0.5 | 0.2 | 997 |
| RIGNYKLNTDHAGSN | 0.7 | 0.5 | 0.6 | 0.3 | 0.5 | 0.6 | 0.1 | 0.6 | 0.6 | 998 |
| 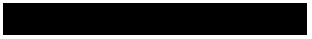 | 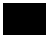 | 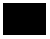 | 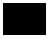 | 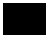 | 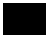 | 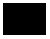 | 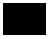 | 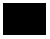 | 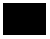 | 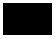 |
| 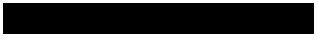 | 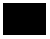 | 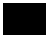 | 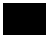 | 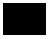 | 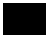 | 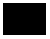 | 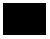 | 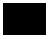 | 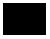 | 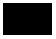 |
| 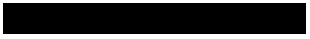 | 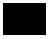 | 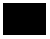 | 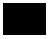 | 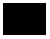 | 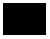 | 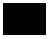 | 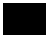 | 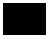 | 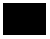 | 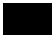 |
| 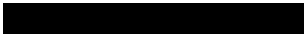 | 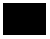 | 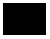 | 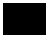 | 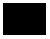 | 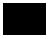 | 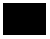 | 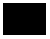 | 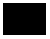 | 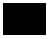 | 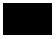 |
| 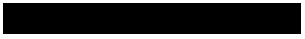 | 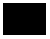 | 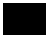 | 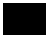 | 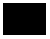 | 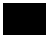 | 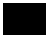 | 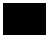 | 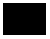 | 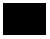 | 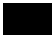 |
| 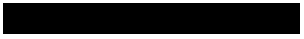 | 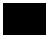 | 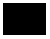 | 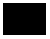 | 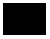 | 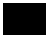 | 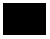 | 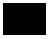 | 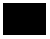 | 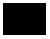 | 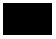 |
| 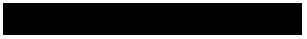 | 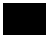 | 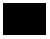 | 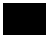 | 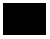 | 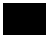 | 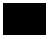 | 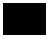 | 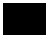 | 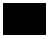 | 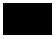 |
| 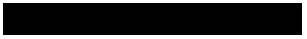 | 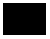 | 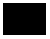 | 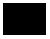 | 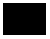 | 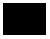 | 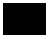 | 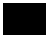 | 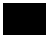 | 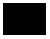 | 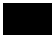 |

TABLE 8
Binding of the sera called SARS-yellow, SARS-green, 1a,
1b, 2, 6, 37, 62 and London to looped/cyclic peptides of protein M
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| 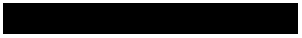 | 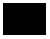 | 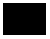 | 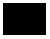 | 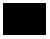 | 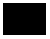 | 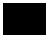 | 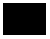 | 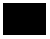 | 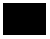 | 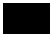 |
| 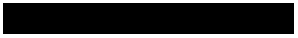 | 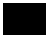 | 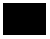 | 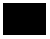 | 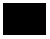 | 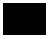 | 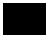 | 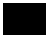 | 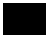 | 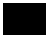 | 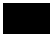 |
| 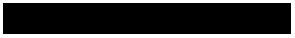 | 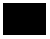 | 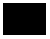 | 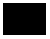 | 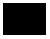 | 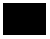 | 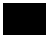 | 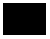 | 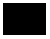 | 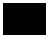 | 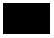 |
| 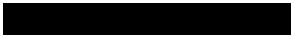 | 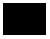 | 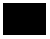 | 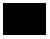 | 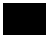 | 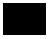 | 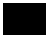 | 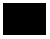 | 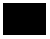 | 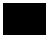 | 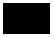 |
| 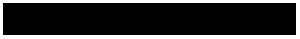 | 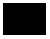 | 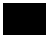 | 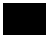 | 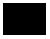 | 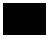 | 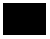 | 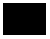 | 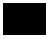 | 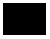 | 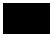 |
| 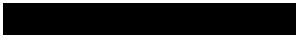 | 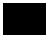 | 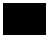 | 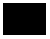 | 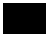 | 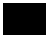 | 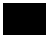 | 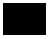 | 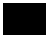 | 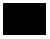 | 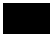 |
| 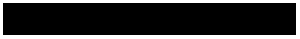 | 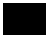 | 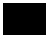 | 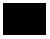 | 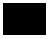 | 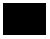 | 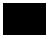 | 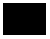 | 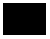 | 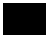 | 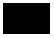 |
| 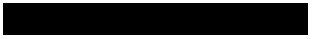 | 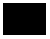 | 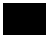 | 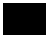 | 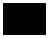 | 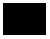 | 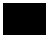 | 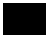 | 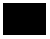 | 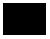 | 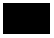 |
| 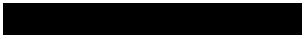 | 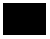 | 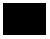 | 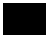 | 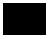 | 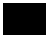 | 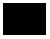 | 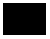 | 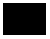 | 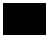 | 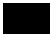 |
| 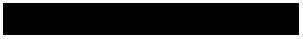 | 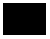 | 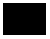 | 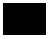 | 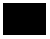 | 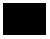 | 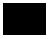 | 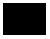 | 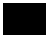 | 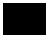 | 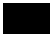 |
| ELKQLLEQWNLVIGF | 0.6 | 0.6 | 0.5 | 0.8 | 0.7 | 0.5 | 0.3 | 0.9 | 0.7 | 879 |
| LKQLLEQWNLVIGFL | 0.6 | 0.6 | 0.4 | 0.8 | 0.5 | 0.7 | 0.2 | 0.7 | 0.4 | 880 |
| KQLLEQWNLVIGFLF | 0.7 | 0.6 | 0.5 | 1.0 | 0.5 | 0.7 | 0.3 | 0.8 | 0.4 | 881 |
| QLLEQWNLVIGFLFL | 0.5 | 0.5 | 0.4 | 0.5 | 0.5 | 0.4 | 0.2 | 0.7 | 0.3 | 882 |
| LLEQWNLVIGFLFLA | 0.5 | 0.2 | 0.4 | 0.4 | 0.4 | 0.4 | 0.2 | 0.4 | 0.2 | 883 |
| LEQWNLVIGELFLAW | 0.6 | 0.3 | 0.7 | 0.5 | 0.7 | 0.6 | 0.2 | 0.6 | 0.3 | 884 |
| EQWNLVIGFLFLAWI | 0.7 | 0.6 | 0.6 | 0.4 | 0.7 | 0.7 | 0.2 | 0.6 | 0.4 | 885 |
| QWNLVIGFLFLAWIM | 0.7 | 0.6 | 0.6 | 0.6 | 0.5 | 0.7 | 0.2 | 0.8 | 0.3 | 886 |
| WNLVIGFLFLAWIML | 0.6 | 0.5 | 0.6 | 0.4 | 0.6 | 0.6 | 0.2 | 0.7 | 0.3 | 887 |
| NLVIGFLFLAWIMLL | 0.7 | 0.6 | 0.5 | 0.6 | 0.6 | 0.7 | 0.3 | 0.5 | 0.3 | 888 |
| LVIGFLFLAWIMLLQ | 0.7 | 0.6 | 0.8 | 0.5 | 0.7 | 0.7 | 0.2 | 0.7 | 0.3 | 889 |
| VIGFLFLAWIMLLQF | 0.7 | 0.4 | 0.5 | 0.5 | 0.5 | 0.7 | 0.2 | 0.6 | 0.3 | 890 |
| IGFLFLAWIMLLQFA | 0.8 | 0.7 | 0.6 | 0.9 | 0.7 | 0.8 | 0.3 | 0.5 | 0.3 | 891 |
| GFLFLAWIMLLQFAY | 0.6 | 0.6 | 0.5 | 0.6 | 0.6 | 0.6 | 0.2 | 0.4 | 0.3 | 892 |
| FLFLAWIMLLQFAYS | 0.8 | 0.7 | 0.6 | 0.9 | 0.7 | 0.8 | 0.3 | 0.4 | 0.3 | 893 |
| LFLAWIMLLQFAYSN | 0.8 | 0.6 | 0.6 | 0.7 | 0.7 | 0.7 | 0.3 | 0.6 | 0.3 | 894 |
| FLAWIMLLQFAYSNR | 0.8 | 0.8 | 0.6 | 0.9 | 0.7 | 0.6 | 0.7 | 0.7 | 0.5 | 895 |
| LAWIMLLQFAYSNRN | 0.7 | 0.7 | 0.4 | 0.7 | 0.6 | 0.6 | 0.2 | 0.3 | 0.4 | 896 |
| AWIMLLQFAYSNRNR | 0.7 | 0.7 | 0.6 | 1.1 | 0.6 | 0.6 | 0.6 | 0.7 | 0.4 | 897 |
| WIMLLQFAYSNRNRF | 0.7 | 0.6 | 0.4 | 0.8 | 0.4 | 0.6 | 0.2 | 0.3 | 0.2 | 898 |
| IMLLQFAYSNRNRFL | 0.6 | 0.4 | 0.6 | 1.0 | 0.6 | 0.5 | 0.6 | 0.6 | 0.3 | 899 |
| MLLQFAYSNRNRFLY | 0.8 | 0.2 | 0.6 | 1.3 | 0.6 | 0.5 | 0.3 | 0.3 | 0.2 | 900 |
| LLQFAYSNRNRFLYI | 0.7 | 0.5 | 0.6 | 0.6 | 0.5 | 0.4 | 0.2 | 0.5 | 0.1 | 901 |
| LQFAYSNRNRFLYII | 0.7 | 0.5 | 0.5 | 0.7 | 0.6 | 0.6 | 0.2 | 0.5 | 0.4 | 902 |
| 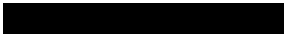 | 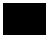 | 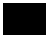 | 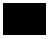 | 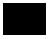 | 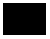 | 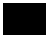 | 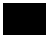 | 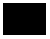 | 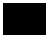 | 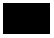 |
| 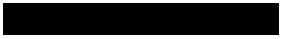 | 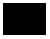 | 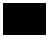 | 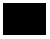 | 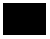 | 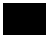 | 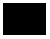 | 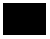 | 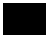 | 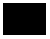 | 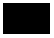 |
| 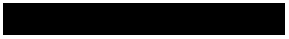 | 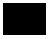 | 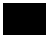 | 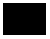 | 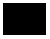 | 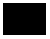 | 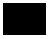 | 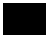 | 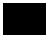 | 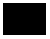 | 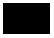 |
| 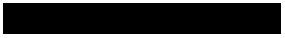 | 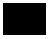 | 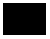 | 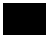 | 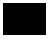 | 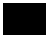 | 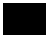 | 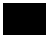 | 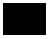 | 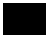 | 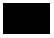 |
| 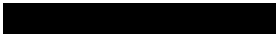 | 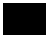 | 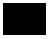 | 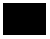 | 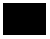 | 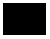 | 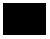 | 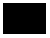 | 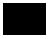 | 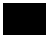 | 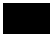 |
| 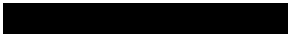 | 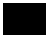 | 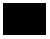 | 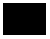 | 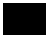 | 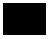 | 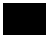 | 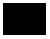 | 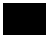 | 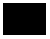 | 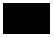 |
| 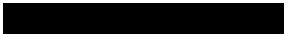 | 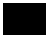 | 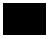 | 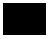 | 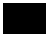 | 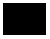 | 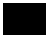 | 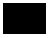 | 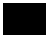 | 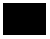 | 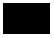 |
| 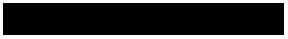 | 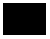 | 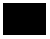 | 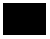 | 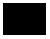 | 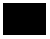 | 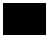 | 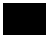 | 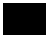 | 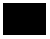 | 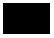 |
| 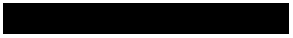 | 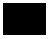 | 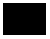 | 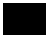 | 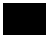 | 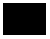 | 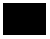 | 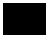 | 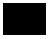 | 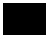 | 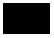 |
| 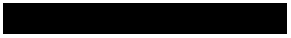 | 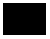 | 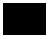 | 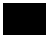 | 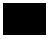 | 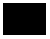 | 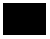 | 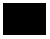 | 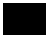 | 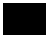 | 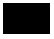 |
| LYIIKLVFLWLLWPV | 0.6 | 0.5 | 0.5 | 0.7 | 0.6 | 0.6 | 0.2 | 0.3 | 0.3 | 903 |
| YIIKLVFLWLLWPVT | 0.8 | 0.8 | 0.6 | 0.8 | 0.7 | 0.7 | 0.3 | 0.5 | 0.4 | 904 |
| IIKLVFLWLLWPVTL | 0.6 | 0.7 | 0.5 | 0.5 | 0.5 | 0.6 | 0.2 | 0.4 | 0.3 | 905 |
| IKLVFLWLLWPVTLA | 0.7 | 0.8 | 0.6 | 0.9 | 0.8 | 0.8 | 0.3 | 0.5 | 0.3 | 906 |
| KLVFLWLLWPVTLAC | 0.6 | 0.2 | 0.6 | 0.7 | 0.6 | 0.6 | 0.2 | 0.5 | 0.3 | 907 |
| LVFLWLLWPVTLACF | 0.6 | 0.5 | 0.7 | 0.6 | 0.7 | 0.7 | 0.2 | 0.4 | 0.3 | 908 |
| VFLWLLWPVTLACFV | 0.7 | 0.0 | 0.7 | 0.5 | 0.7 | 0.7 | 0.2 | 0.8 | 0.3 | 909 |
| FLWLLWPVTLACFVL | 0.6 | 0.5 | 0.6 | 0.4 | 0.6 | 0.6 | 0.2 | 0.7 | 0.2 | 910 |
| LWLLWPVTLACFVLA | 0.7 | 0.4 | 0.6 | 0.5 | 0.7 | 0.6 | 0.2 | 0.6 | 0.2 | 911 |
| WLLWPVTLACFVLAA | 0.7 | 0.3 | 0.7 | 0.5 | 0.7 | 0.7 | 0.2 | 0.7 | 0.3 | 912 |
| LLWPVTLACFVLAAV | 0.9 | 0.5 | 0.9 | 0.6 | 0.8 | 0.8 | 0.3 | 0.9 | 0.4 | 913 |
| LWPVTLACFVLAAVY | 0.7 | 0.5 | 0.7 | 0.6 | 0.7 | 0.6 | 0.2 | 0.5 | 0.2 | 914 |
| WPVTLACFVLAAVYR | 0.8 | 0.6 | 0.8 | 1.1 | 0.8 | 1.1 | 0.4 | 0.4 | 0.2 | 915 |
| PVTLACFVLAAVYRI | 0.7 | 0.6 | 0.6 | 0.7 | 0.7 | 0.7 | 0.2 | 0.6 | 0.2 | 916 |
| VTLACFVLAAVYRIN | 0.8 | 0.6 | 0.6 | 0.8 | 0.7 | 0.7 | 0.3 | 0.6 | 0.2 | 917 |
| TLACFVLAAVYRINW | 0.7 | 0.5 | 0.6 | 0.7 | 0.6 | 0.6 | 0.3 | 0.5 | 0.2 | 918 |
| LACFVLAAVYRINWV | 0.7 | 0.5 | 0.9 | 0.7 | 0.6 | 0.7 | 0.3 | 0.4 | 0.3 | 919 |
| ACFVLAAVYRINWVT | 0.7 | 0.7 | 0.5 | 1.0 | 0.7 | 0.6 | 0.3 | 0.5 | 0.3 | 920 |
| CFVLAAVYRINWVTG | 0.6 | 0.6 | 0.5 | 0.8 | 0.6 | 0.7 | 0.2 | 0.4 | 0.3 | 921 |
| FVLAAVYRINWVTGG | 0.7 | 0.6 | 0.5 | 0.8 | 0.5 | 0.6 | 0.3 | 0.4 | 0.3 | 922 |
| VLAAVYRINWVTGGI | 0.6 | 0.5 | 0.6 | 0.7 | 0.6 | 0.5 | 0.2 | 0.6 | 0.2 | 923 |
| LAAVYRINWVTGGIA | 0.8 | 0.2 | 0.5 | 0.7 | 0.5 | 0.5 | 0.2 | 0.3 | 0.3 | 924 |
| AAVYRINWVTGGIAI | 0.6 | 0.4 | 0.6 | 0.5 | 0.7 | 0.4 | 0.2 | 0.7 | 0.0 | 925 |
| AVYRINWVTGGIAIA | 0.8 | 0.4 | 0.6 | 0.6 | 0.7 | 0.7 | 0.2 | 0.7 | 0.4 | 926 |
| VYRINWVTGGIAIAM | 0.7 | 0.4 | 0.6 | 0.5 | 0.7 | 1.1 | 0.2 | 0.8 | 0.3 | 927 |
| YRINWVTGGIAIAMA | 0.6 | 0.4 | 0.6 | 0.5 | 0.5 | 0.6 | 0.2 | 0.8 | 0.0 | 928 |
| RINWVTGGIAIAMAC | 0.7 | 0.2 | 0.6 | 0.4 | 0.7 | 0.7 | 0.2 | 0.7 | 0.3 | 929 |
| 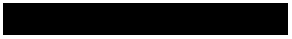 | 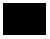 | 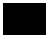 | 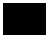 | 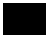 | 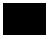 | 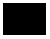 | 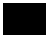 | 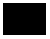 | 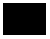 | 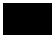 |
| 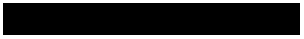 | 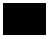 | 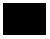 | 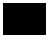 | 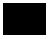 | 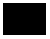 | 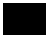 | 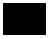 | 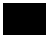 | 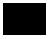 | 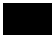 |
| 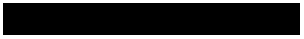 | 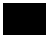 | 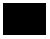 | 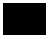 | 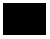 | 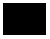 | 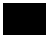 | 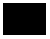 | 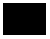 | 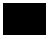 | 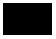 |
| 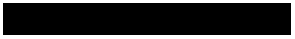 | 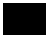 | 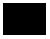 | 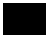 | 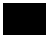 | 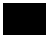 | 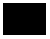 | 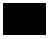 | 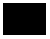 | 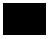 | 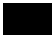 |
| 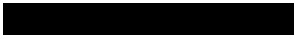 | 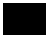 | 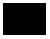 | 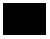 | 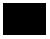 | 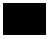 | 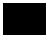 | 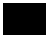 | 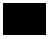 | 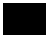 | 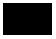 |
| 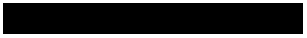 | 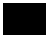 | 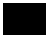 | 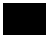 | 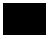 | 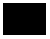 | 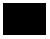 | 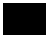 | 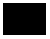 | 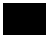 | 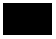 |
| 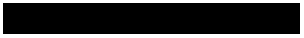 | 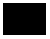 | 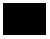 | 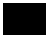 | 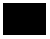 | 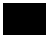 | 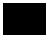 | 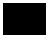 | 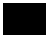 | 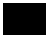 | 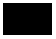 |
| 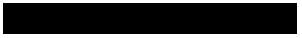 | 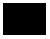 | 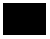 | 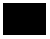 | 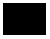 | 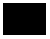 | 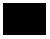 | 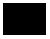 | 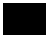 | 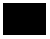 | 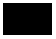 |
| AIAMACIVGLMWLSY | 0.5 | 0.3 | 0.4 | 0.6 | 0.5 | 0.4 | 0.2 | 0.8 | 4.4 | 930 |
| IAMACIVGLMWLSYF | 0.6 | 0.4 | 0.4 | 0.7 | 0.4 | 0.4 | 0.2 | 0.6 | 0.3 | 931 |
| AMACIVGLMWLSYFV | 0.7 | 0.7 | 0.6 | 0.9 | 0.6 | 0.5 | 0.2 | 0.6 | 0.4 | 932 |
| MACIVGLMWLSYFVA | 0.7 | 0.4 | 0.4 | 0.5 | 0.4 | 0.5 | 0.2 | 0.4 | 0.2 | 933 |
| ACIVGLMWLSYFVAS | 0.6 | 0.3 | 0.5 | 0.6 | 0.5 | 0.4 | 0.2 | 0.4 | 0.2 | 934 |
| CIVGLMWLSYFVASF | 0.5 | 0.2 | 0.4 | 0.4 | 0.6 | 0.4 | 0.2 | 0.5 | 0.0 | 935 |
| IVGLMWLSYFVASFR | 0.7 | 0.5 | 1.0 | 0.7 | 0.7 | 0.7 | 0.2 | 0.6 | 0.1 | 936 |
| VGLMWLSYFVASFRL | 0.6 | 0.3 | 0.5 | 0.5 | 0.6 | 0.5 | 0.2 | 0.8 | 0.2 | 937 |
| GLMWLSYFVASFRLF | 0.6 | 0.2 | 0.6 | 0.8 | 0.5 | 0.6 | 0.3 | 0.7 | 0.3 | 938 |
| 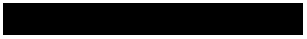 | 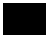 | 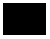 | 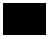 | 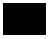 | 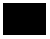 | 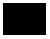 | 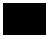 | 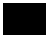 | 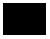 | 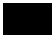 |
| 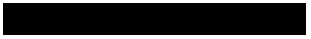 | 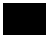 | 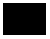 | 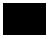 | 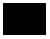 | 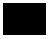 | 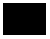 | 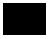 | 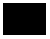 | 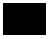 | 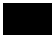 |
| 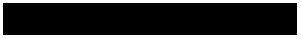 | 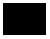 | 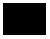 | 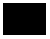 | 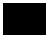 | 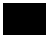 | 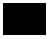 | 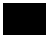 | 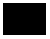 | 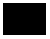 | 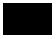 |
| 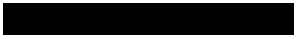 | 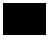 | 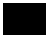 | 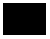 | 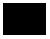 | 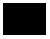 | 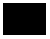 | 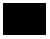 | 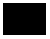 | 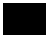 | 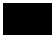 |
| 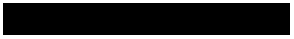 | 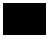 | 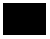 | 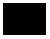 | 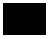 | 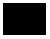 | 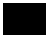 | 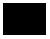 | 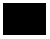 | 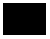 | 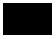 |
| 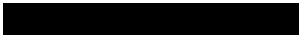 | 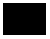 | 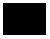 | 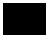 | 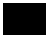 | 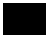 | 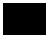 | 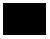 | 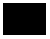 | 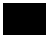 | 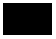 |
| 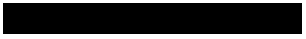 | 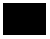 | 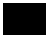 | 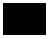 | 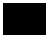 | 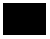 | 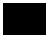 | 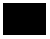 | 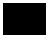 | 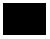 | 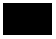 |
| 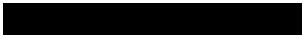 | 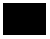 | 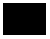 | 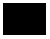 | 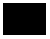 | 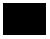 | 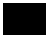 | 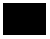 | 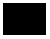 | 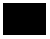 | 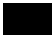 |
| ASFRLFARTRSMWSF | 0.6 | 0.5 | 0.5 | 0.9 | 0.5 | 0.5 | 0.2 | 0.6 | 0.2 | 939 |
| SFRLFARTRSMWSFN | 0.6 | 0.5 | 0.5 | 0.8 | 0.6 | 0.6 | 0.2 | 0.5 | 0.3 | 940 |
| FRLFARTRSMWSFNP | 0.7 | 0.5 | 0.6 | 0.8 | 0.6 | 0.6 | 0.3 | 0.5 | 0.3 | 941 |
| RLFARTRSMWSFNPE | 0.7 | 0.8 | 0.5 | 0.8 | 0.6 | 0.6 | 0.2 | 0.4 | 0.4 | 942 |
| LFARTRSMWSFNPET | 0.7 | 0.5 | 0.6 | 0.5 | 0.7 | 0.6 | 0.2 | 0.7 | 0.3 | 943 |
| FARTRSMWSFNPETN | 0.9 | 0.6 | 0.6 | 0.7 | 0.9 | 0.8 | 0.3 | 0.5 | 0.0 | 944 |
| ARTRSMWSPNPETNI | 0.7 | 0.1 | 0.9 | 0.6 | 0.8 | 0.8 | 0.2 | 0.7 | 0.1 | 945 |
| RTRSMWSFNPETNIL | 0.7 | 0.3 | 0.8 | 0.7 | 0.9 | 0.9 | 0.2 | 0.7 | 0.0 | 946 |
| TRSMWSFNPETNILL | 1.0 | 0.4 | 0.8 | 0.9 | 1.1 | 1.1 | 0.3 | 0.7 | 0.5 | 947 |
| RSMWSFNPETNILLN | 0.8 | 0.4 | 0.8 | 0.7 | 0.8 | 0.8 | 0.2 | 0.7 | 0.4 | 948 |
| SMWSFNPETNILLNV | 0.7 | 0.1 | 0.8 | 0.6 | 0.8 | 0.7 | 0.2 | 0.7 | 0.2 | 949 |
| MWSFNPETNILLNVP | 0.8 | 0.7 | 0.9 | 0.5 | 0.8 | 0.8 | 0.2 | 0.6 | 0.3 | 950 |
| WSFNPETNILLNVPL | 0.8 | 0.5 | 0.6 | 0.5 | 0.7 | 0.7 | 0.2 | 0.8 | 0.2 | 951 |
| SFNPETNILLNVPLR | 0.8 | 0.7 | 0.9 | 0.8 | 0.7 | 0.8 | 0.3 | 1.0 | 0.3 | 952 |
| FNPETNILLNVPLRG | 0.8 | 0.8 | 0.7 | 0.7 | 0.9 | 0.8 | 0.3 | 0.8 | 0.3 | 953 |
| NPETNILLNVPLRGT | 0.8 | 0.7 | 0.9 | 0.7 | 0.7 | 0.7 | 0.3 | 0.7 | 0.4 | 954 |
| PETNILLNVPLRGTI | 0.6 | 0.6 | 0.7 | 0.9 | 0.8 | 0.8 | 0.3 | 0.6 | 0.0 | 955 |
| ETNILLNVPLRGTIV | 0.5 | 0.6 | 0.7 | 0.8 | 0.4 | 0.5 | 0.2 | 0.5 | 0.2 | 956 |
| TNILLNVPLRGTIVT | 0.6 | 0.4 | 0.6 | 0.8 | 0.7 | 0.6 | 0.2 | 0.4 | 0.2 | 957 |
| 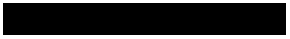 | 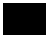 | 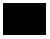 | 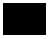 | 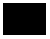 | 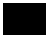 | 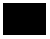 | 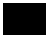 | 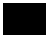 | 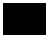 | 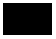 |
| 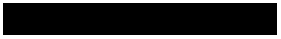 | 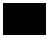 | 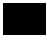 | 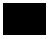 | 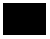 | 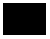 | 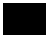 | 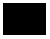 | 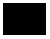 | 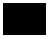 | 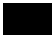 |
| 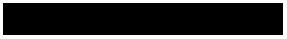 | 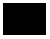 | 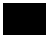 | 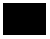 | 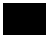 | 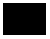 | 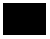 | 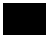 | 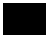 | 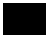 | 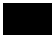 |
| 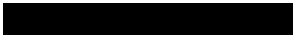 | 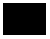 | 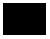 | 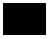 | 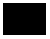 | 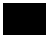 | 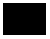 | 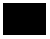 | 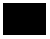 | 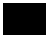 | 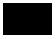 |
| 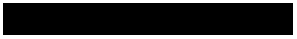 | 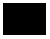 | 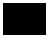 | 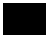 | 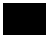 | 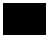 | 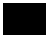 | 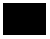 | 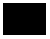 | 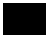 | 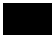 |
| 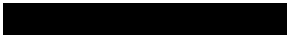 | 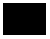 | 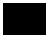 | 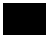 | 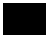 | 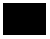 | 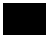 | 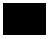 | 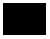 | 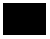 | 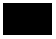 |
| 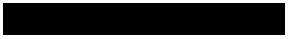 | 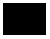 | 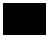 | 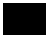 | 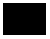 | 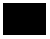 | 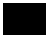 | 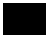 | 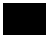 | 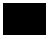 | 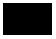 |
| 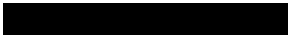 | 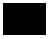 | 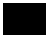 | 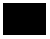 | 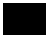 | 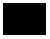 | 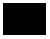 | 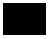 | 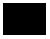 | 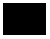 | 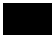 |
| 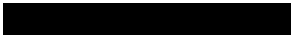 | 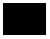 | 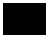 | 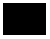 | 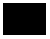 | 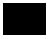 | 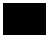 | 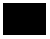 | 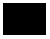 | 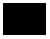 | 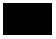 |
| 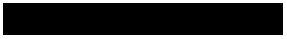 | 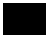 | 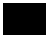 | 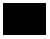 | 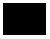 | 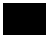 | 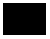 | 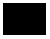 | 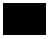 | 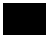 | 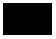 |
| 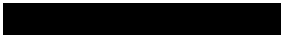 | 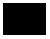 | 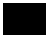 | 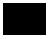 | 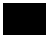 | 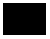 | 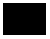 | 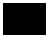 | 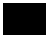 | 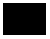 | 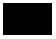 |
| 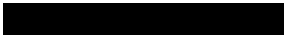 | 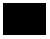 | 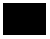 | 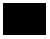 | 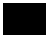 | 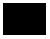 | 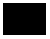 | 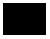 | 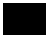 | 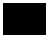 | 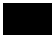 |
| 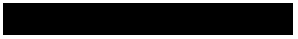 | 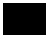 | 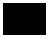 | 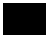 | 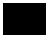 | 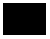 | 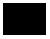 | 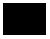 | 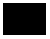 | 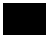 | 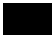 |
| 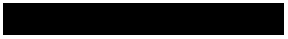 | 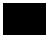 | 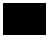 | 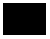 | 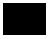 | 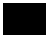 | 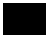 | 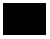 | 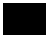 | 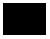 | 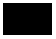 |
| 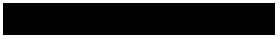 | 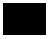 | 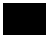 | 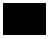 | 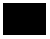 | 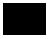 | 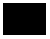 | 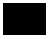 | 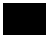 | 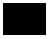 | 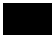 |
| PLMESELVIGAVIIR | 0.7 | 0.6 | 0.7 | 0.8 | 0.8 | 0.8 | 0.2 | 0.7 | 0.3 | 958 |
| LMESELVIGAVIIRG | 0.6 | 0.6 | 0.5 | 0.4 | 0.6 | 0.6 | 0.2 | 0.5 | 0.3 | 959 |
| MESELVIGAVIIRGH | 0.7 | 0.5 | 0.6 | 0.7 | 0.7 | 0.6 | 0.2 | 0.6 | 0.2 | 960 |
| ESELVIGAVIIRGHL | 0.6 | 0.5 | 0.6 | 0.5 | 0.5 | 0.5 | 0.2 | 0.4 | 0.2 | 961 |
| SELVIGAVIIRGHLR | 0.8 | 0.7 | 0.8 | 0.6 | 0.8 | 0.8 | 0.2 | 0.9 | 0.3 | 962 |
| ELVIGAVIIRGHLRM | 0.8 | 0.4 | 0.7 | 0.7 | 0.7 | 0.6 | 0.2 | 0.6 | 0.3 | 963 |
| LVIGAVIIRGHLRMA | 0.8 | 0.4 | 0.8 | 1.2 | 0.7 | 0.8 | 0.4 | 0.7 | 0.3 | 964 |
| 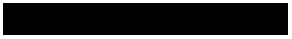 | 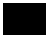 | 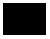 | 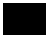 | 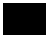 | 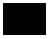 | 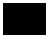 | 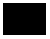 | 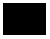 | 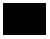 | 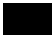 |
| 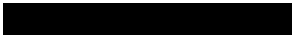 | 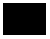 | 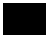 | 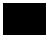 | 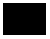 | 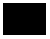 | 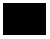 | 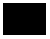 | 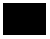 | 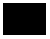 | 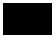 |
| 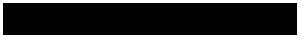 | 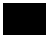 | 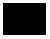 | 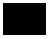 | 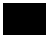 | 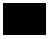 | 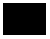 | 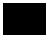 | 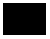 | 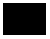 | 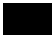 |
| 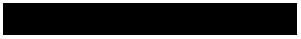 | 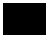 | 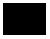 | 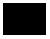 | 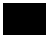 | 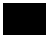 | 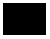 | 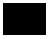 | 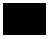 | 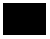 | 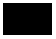 |
| 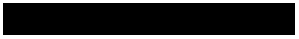 | 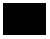 | 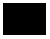 | 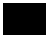 | 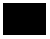 | 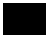 | 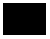 | 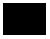 | 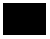 | 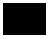 | 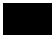 |
| 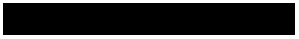 | 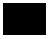 | 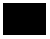 | 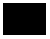 | 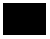 | 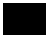 | 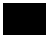 | 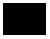 | 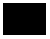 | 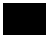 | 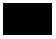 |
| 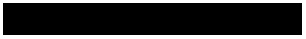 | 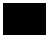 | 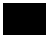 | 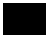 | 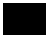 | 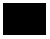 | 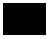 | 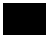 | 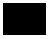 | 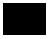 | 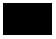 |
| 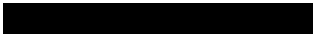 | 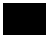 | 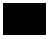 | 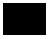 | 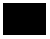 | 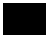 | 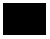 | 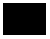 | 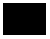 | 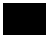 | 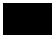 |
| 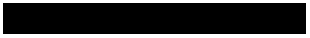 | 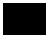 | 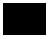 | 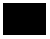 | 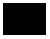 | 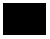 | 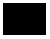 | 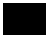 | 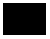 | 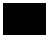 | 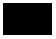 |
| 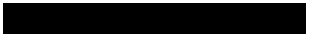 | 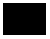 | 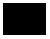 | 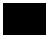 | 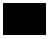 | 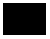 | 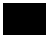 | 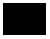 | 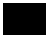 | 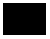 | 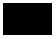 |
| 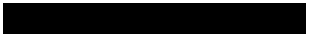 | 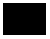 | 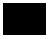 | 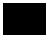 | 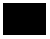 | 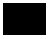 | 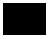 | 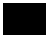 | 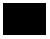 | 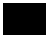 | 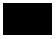 |
| 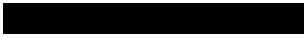 | 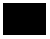 | 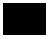 | 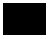 | 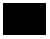 | 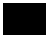 | 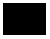 | 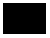 | 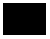 | 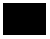 | 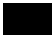 |
| 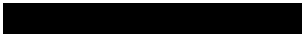 | 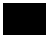 | 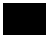 | 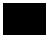 | 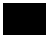 | 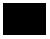 | 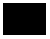 | 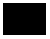 | 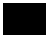 | 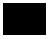 | 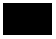 |
| 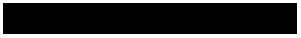 | 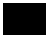 | 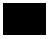 | 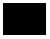 | 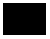 | 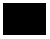 | 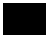 | 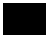 | 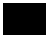 | 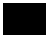 | 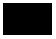 |
| 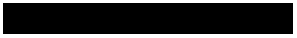 | 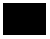 | 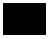 | 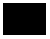 | 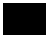 | 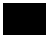 | 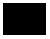 | 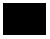 | 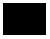 | 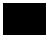 | 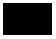 |
| 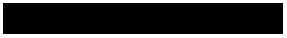 | 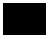 | 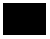 | 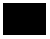 | 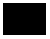 | 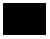 | 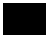 | 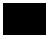 | 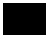 | 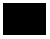 | 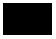 |
| 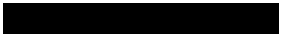 | 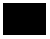 | 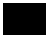 | 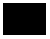 | 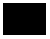 | 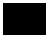 | 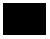 | 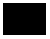 | 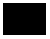 | 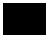 | 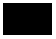 |
| 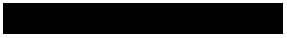 | 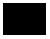 | 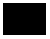 | 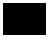 | 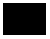 | 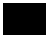 | 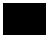 | 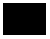 | 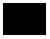 | 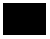 | 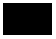 |
| 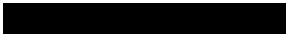 | 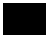 | 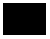 | 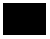 | 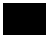 | 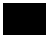 | 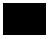 | 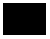 | 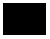 | 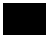 | 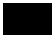 |
| RCDIKDLPKEITVAT | 0.6 | 0.4 | 0.6 | 0.6 | 0.6 | 0.6 | 0.2 | 0.7 | 0.4 | 965 |
| GDIKDLPKEITVATS | 0.6 | 0.3 | 0.6 | 0.5 | 0.6 | 0.6 | 0.2 | 0.6 | 0.4 | 966 |
| DIKDLPKEITVATSR | 0.6 | 0.6 | 0.7 | 0.8 | 0.9 | 0.7 | 0.3 | 0.7 | 0.4 | 967 |
| IKDLPKEITVATSRT | 0.4 | 0.4 | 0.5 | 0.4 | 0.6 | 0.6 | 0.2 | 0.9 | 0.3 | 968 |
| KDLPKEITVATSRTL | 0.6 | 0.6 | 0.7 | 1.1 | 0.6 | 0.6 | 1.1 | 0.9 | 0.3 | 969 |
| DLPKEITVATSRTLS | 0.5 | 0.5 | 0.6 | 0.8 | 0.7 | 0.6 | 0.4 | 0.7 | 0.4 | 970 |
| LPKEITVATSRTLSY | 0.6 | 0.4 | 0.6 | 0.6 | 0.6 | 0.5 | 0.2 | 0.5 | 0.3 | 971 |
| PKEITVATSRTLSYY | 0.6 | 0.6 | 0.6 | 0.7 | 0.6 | 0.6 | 0.2 | 0.4 | 0.4 | 972 |
| KEITVATSRTLSYYK | 0.6 | 0.5 | 0.8 | 1.3 | 0.6 | 0.5 | 0.4 | 0.5 | 0.2 | 973 |
| EITVATSRTLSYYKL | 0.6 | 0.4 | 0.5 | 0.5 | 0.5 | 0.5 | 0.2 | 0.5 | 0.3 | 974 |
| ITVATSRTLSYYKLG | 1.0 | 0.6 | 0.8 | 1.1 | 0.3 | 0.6 | 0.4 | 0.4 | 0.4 | 975 |
| TVATSRTLSYYKLGA | 0.8 | 0.4 | 0.6 | 1.1 | 0.5 | 0.5 | 0.3 | 0.5 | 0.4 | 976 |
| VATSRTLSYYKLGAS | 0.8 | 0.6 | 0.7 | 0.6 | 0.7 | 0.6 | 0.2 | 0.6 | 0.3 | 977 |
| ATSRTLSYYKLGASQ | 0.8 | 0.2 | 0.6 | 0.8 | 0.6 | 0.5 | 0.3 | 0.7 | 0.3 | 978 |
| TSRTLSYYKLGASQR | 0.7 | 0.3 | 0.7 | 1.3 | 0.6 | 0.6 | 0.7 | 0.9 | 0.3 | 979 |
| SRTLSYYKLGASQRV | 0.9 | 0.3 | 0.7 | 0.9 | 0.8 | 0.7 | 0.3 | 0.8 | 0.4 | 980 |
| RTLSYYKLGASQRVG | 0.8 | 0.5 | 0.7 | 1.5 | 0 6 | 0 7 | 1.0 | 0.8 | 0.4 | 981 |
| 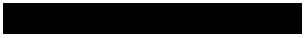 | 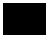 | 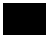 | 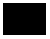 | 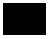 | 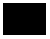 | 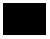 | 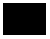 | 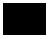 | 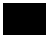 | 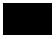 |
| 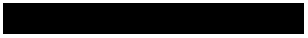 | 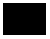 | 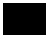 | 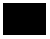 | 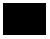 | 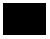 | 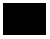 | 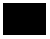 | 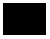 | 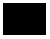 | 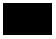 |
| 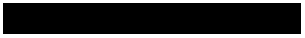 | 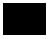 | 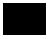 | 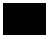 | 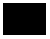 | 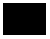 | 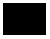 | 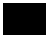 | 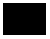 | 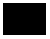 | 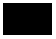 |
| 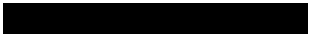 | 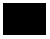 | 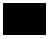 | 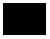 | 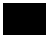 | 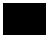 | 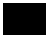 | 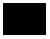 | 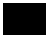 | 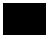 | 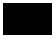 |
| 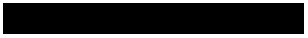 | 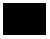 | 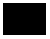 | 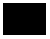 | 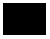 | 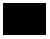 | 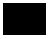 | 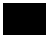 | 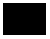 | 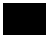 | 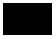 |
| 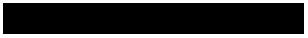 | 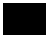 | 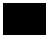 | 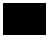 | 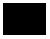 | 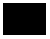 | 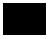 | 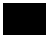 | 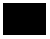 | 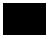 | 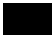 |
| 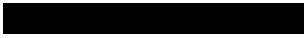 | 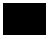 | 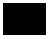 | 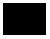 | 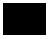 | 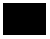 | 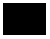 | 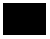 | 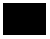 | 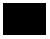 | 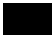 |
| 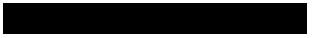 | 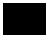 | 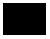 | 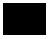 | 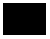 | 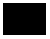 | 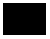 | 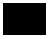 | 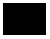 | 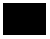 | 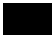 |
| 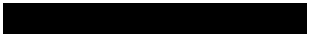 | 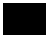 | 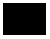 | 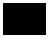 | 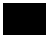 | 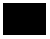 | 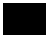 | 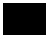 | 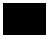 | 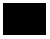 | 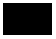 |
| SQRVGTDSGFAAYNR | 0.5 | 0.4 | 0.7 | 0.6 | 0.2 | 0.5 | 0.2 | 0.6 | 0.4 | 982 |
| QRVGTDSGFAAYNRY | 0.6 | 0.5 | 0.5 | 0.6 | 0.6 | 0.5 | 0.2 | 0.6 | 0.3 | 983 |
| RVGTDSGFAAYNRYR | 0.8 | 0.5 | 0.6 | 1.4 | 0.7 | 0.6 | 0.4 | 0.5 | 0.3 | 984 |
| VGTDSGFAAYNRYRI | 0.7 | 0.4 | 0.7 | 0.6 | 0.7 | 0.8 | 0.2 | 0.5 | 0.4 | 985 |
| GTDSGFAAYNRYRIG | 0.8 | 0.5 | 0.6 | 0.8 | 0.7 | 0.6 | 0.3 | 0.9 | 0.4 | 986 |
| TDSGFAAYNRYRIGN | 0.7 | 0.5 | 0.6 | 0.9 | 0.7 | 0.7 | 0.2 | 0.8 | 0.1 | 987 |
| DSGFAAYNRYRIGNY | 0.8 | 0.6 | 0.6 | 0.6 | 0.7 | 0.6 | 0.2 | 0.8 | 0.4 | 988 |
| SGFAAYNRYRIGNYK | 0.9 | 0.6 | 0.9 | 1.4 | 0.7 | 0.7 | 0.3 | 0.7 | 0.2 | 989 |
| GFAAYNRYRIGNYKL | 0.7 | 0.2 | 0.6 | 0.7 | 0.6 | 0.5 | 0.2 | 0.5 | 0.2 | 990 |
| FAAYNRYRIGNYKLN | 0.8 | 0.4 | 0.7 | 1.3 | 0.6 | 0.6 | 0.7 | 0.8 | 0.2 | 991 |
| AAYNRYRTGNYKLNT | 0.7 | 0.5 | 0.8 | 1.3 | 0.5 | 0.7 | 0.5 | 0.7 | 0.3 | 992 |
| AYNRYRIGNYKLNTD | 0.8 | 0.7 | 0.5 | 1.0 | 0.6 | 0.6 | 0.4 | 0.6 | 0.2 | 993 |
| YNRYRIGNYKLNTDH | 0.8 | 0.7 | 0.7 | 1.1 | 0.5 | 0.6 | 0.6 | 0.7 | 0.3 | 994 |
| NRYRIGNYKLNTDHA | 0.8 | 0.6 | 0.5 | 0.7 | 0.6 | 0.5 | 0.4 | 0.9 | 0.3 | 995 |
| RYRIGNYKLNTDHAG | 0.7 | 0.6 | 0.6 | 0.9 | 0.6 | 0.6 | 0.4 | 0.8 | 0.3 | 996 |
| YRIGNYKLNTDHAGS | 0.8 | 0.6 | 0.5 | 0.8 | 0.7 | 0.6 | 0.4 | 0.6 | 0.3 | 997 |
| RIGNYKLNTDHAGSN | 0.6 | 0.5 | 0.5 | 0.7 | 0.6 | 0.6 | 0.4 | 0.6 | 0.2 | 998 |
| 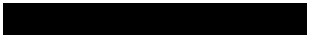 | 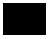 | 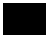 | 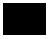 | 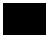 | 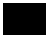 | 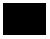 | 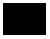 | 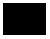 | 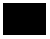 | 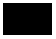 |
| 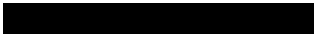 | 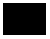 | 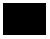 | 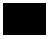 | 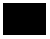 | 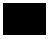 | 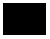 | 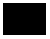 | 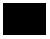 | 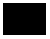 | 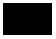 |
| 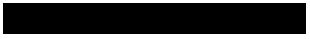 | 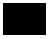 | 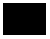 | 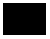 | 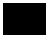 | 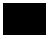 | 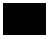 | 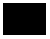 | 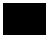 | 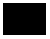 | 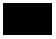 |
| 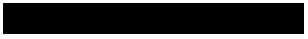 | 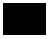 | 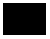 | 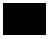 | 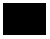 | 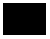 | 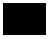 | 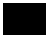 | 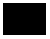 | 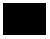 | 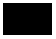 |
| 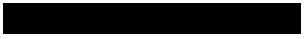 | 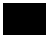 | 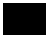 | 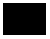 | 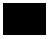 | 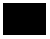 | 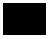 | 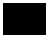 | 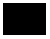 | 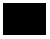 | 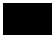 |
| 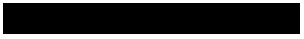 | 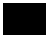 | 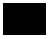 | 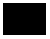 | 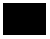 | 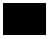 | 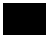 | 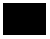 | 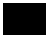 | 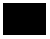 | 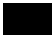 |
| 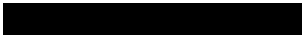 | 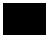 | 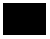 | 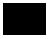 | 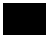 | 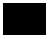 | 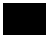 | 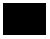 | 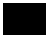 | 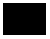 | 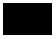 |
| 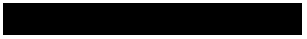 | 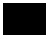 | 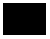 | 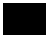 | 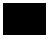 | 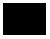 | 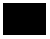 | 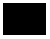 | 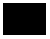 | 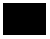 | 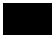 |

TABLE 9
Binding of the sera called SARS-yellow, SARS-green, 1a,
1b, 2, 6, 37, 62 and London to linear peptides of protein X3 of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MFHLVDFQVTIAEIL | 0.8 | 0.7 | 0.7 | 0.5 | 0.7 | 0.8 | 0.6 | 0.3 | 0.7 | 999 |
| FHLVDFQVTIAEILI | 0.8 | 0.6 | 0.7 | 0.4 | 0.7 | 0.6 | 0.9 | 0.5 | 0.6 | 1000 |
| HLVDFQVTIAEILII | 0.8 | 0.6 | 0.7 | 0.5 | 0.7 | 0.6 | 0.5 | 0.4 | 0.6 | 1001 |
| LVDFQVTIAEILIII | 0.8 | 0.6 | 0.7 | 0.5 | 0.6 | 0.6 | 0.7 | 0.4 | 0.6 | 1002 |
| VDFQVTIAETLIIIM | 0.8 | 0.5 | 0.7 | 0.5 | 0.5 | 0.6 | 0.6 | 0.4 | 0.6 | 1003 |
| DFQVTIAEILIIIMR | 0.7 | 0.5 | 0.6 | 0.5 | 0.6 | 0.6 | 0.5 | 0.4 | 0.6 | 1004 |
| FQVTIAEILIIIMRT | 0.7 | 0.3 | 0.6 | 0.6 | 0.3 | 0.5 | 1.1 | 0.5 | 0.7 | 1005 |
| QVTIAEILIIIMRTF | 0.7 | 0.4 | 0.5 | 0.5 | 0.4 | 0.7 | 0.2 | 0.4 | 0.7 | 1006 |
| VTIAEILIIIMRTFR | 0.7 | 0.3 | 0.7 | 0.7 | 0.4 | 0.6 | 0.3 | 0.5 | 0.6 | 1007 |
| TIAEILIIIMRTFRI | 0.8 | 0.3 | 0.7 | 0.7 | 0.6 | 0.7 | 0.3 | 0.3 | 0.7 | 1008 |
| IAETLIITMRTFRIA | 0.8 | 0.5 | 0.6 | 0.0 | 0.6 | 0.7 | 0.3 | 0.3 | 0.7 | 1009 |
| 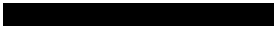 | 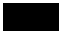 | 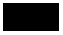 | 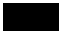 | 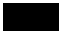 | 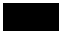 | 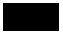 | 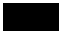 | 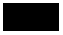 | 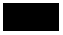 | 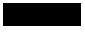 |
| 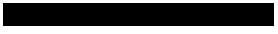 | 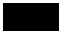 | 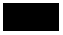 | 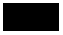 | 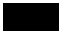 | 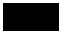 | 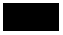 | 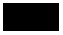 | 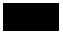 | 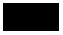 | 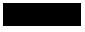 |
| 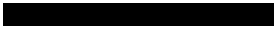 | 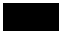 | 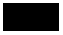 | 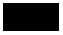 | 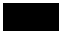 | 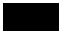 | 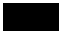 | 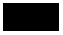 | 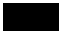 | 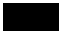 | 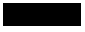 |
| 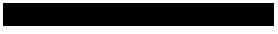 | 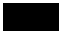 | 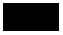 | 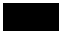 | 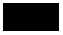 | 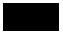 | 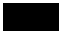 | 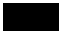 | 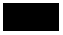 | 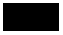 | 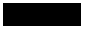 |
| 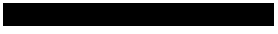 | 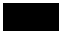 | 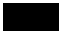 | 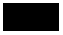 | 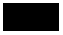 | 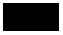 | 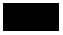 | 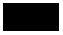 | 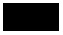 | 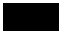 | 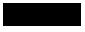 |
| 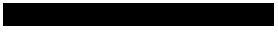 | 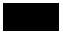 | 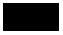 | 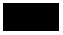 | 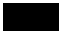 | 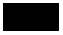 | 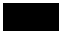 | 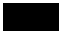 | 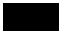 | 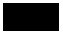 | 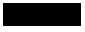 |
| 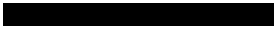 | 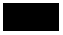 | 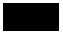 | 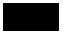 | 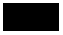 | 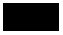 | 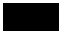 | 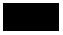 | 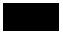 | 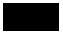 | 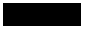 |
| 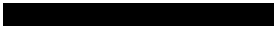 | 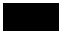 | 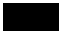 | 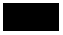 | 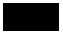 | 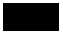 | 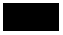 | 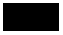 | 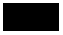 | 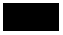 | 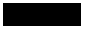 |
| 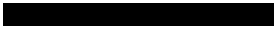 | 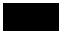 | 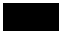 | 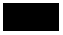 | 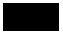 | 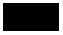 | 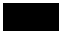 | 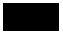 | 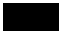 | 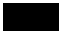 | 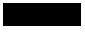 |
| TFRIAIWNLDVIISS | 0.8 | 0.5 | 0.6 | 0.5 | 0.6 | 0.7 | 0.6 | 0.4 | 0.7 | 1010 |
| FRIAIWNLDVIISSI | 0.8 | 0.5 | 0.6 | 0.4 | 0.5 | 0.7 | 0.7 | 0.4 | 0.7 | 1011 |
| RIAIWNLDVIISSIV | 0.7 | 0.3 | 0.6 | 0.5 | 0.5 | 0.7 | 0.4 | 0.3 | 0.7 | 1012 |
| IAIWNLDVIISSIVR | 0.7 | 0.5 | 0.6 | 0.5 | 0.5 | 0.6 | 0.4 | 0.3 | 0.6 | 1013 |
| AIWNLDVIISSTVRQ | 0.8 | 0.4 | 0.6 | 0.5 | 0.6 | 0.6 | 0.3 | 0.3 | 0.6 | 1014 |
| IWNLDVIISSIVRQL | 0.7 | 0.3 | 0.5 | 0.5 | 0.6 | 0.6 | 0.2 | 0.4 | 0.6 | 1015 |
| WNLDVIISSIVRQLF | 0.7 | 0.2 | 0.4 | 0.4 | 0.6 | 0.6 | 0.2 | 0.2 | 0.5 | 1016 |
| NLDVIISSIVRQLFK | 0.7 | 0.2 | 0.4 | 0.6 | 0.6 | 0.6 | 0.3 | 0.3 | 0.7 | 1017 |
| LDVIISSIVRQLFKP | 0.8 | 0.3 | 0.9 | 0.3 | 0.7 | 0.6 | 0.1 | 0.4 | 0.5 | 1018 |
| DVIISSIVRQLFKPL | 0.7 | 0.4 | 0.5 | 0.3 | 0.6 | 0.6 | 0.2 | 0.3 | 0.6 | 1019 |
| 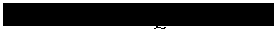 | 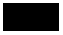 | 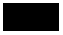 | 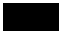 | 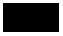 | 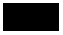 | 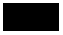 | 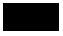 | 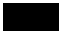 | 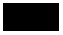 | 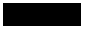 |
| 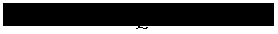 | 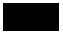 | 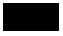 | 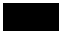 | 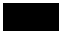 | 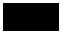 | 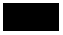 | 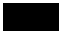 | 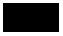 | 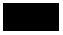 | 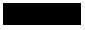 |
| 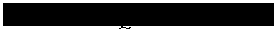 | 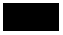 | 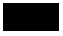 | 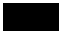 | 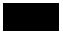 | 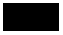 | 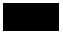 | 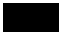 | 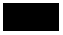 | 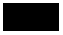 | 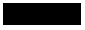 |
| 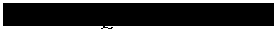 | 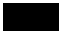 | 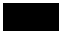 | 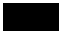 | 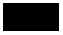 | 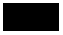 | 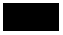 | 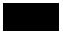 | 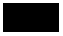 | 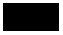 | 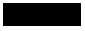 |
| 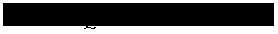 | 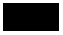 | 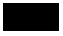 | 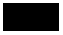 | 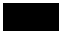 | 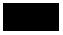 | 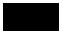 | 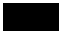 | 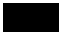 | 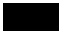 | 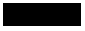 |
| 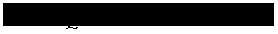 | 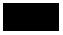 | 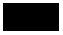 | 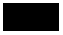 | 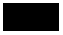 | 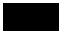 | 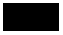 | 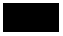 | 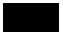 | 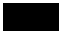 | 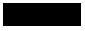 |
| 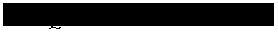 | 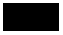 | 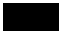 | 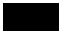 | 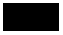 | 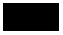 | 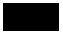 | 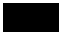 | 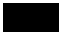 | 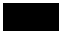 | 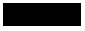 |
| 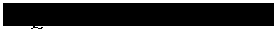 | 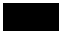 | 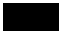 | 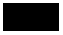 | 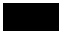 | 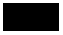 | 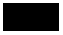 | 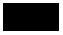 | 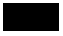 | 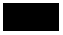 | 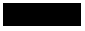 |
| 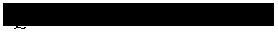 | 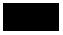 | 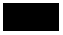 | 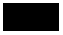 | 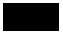 | 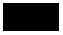 | 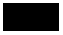 | 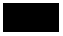 | 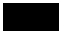 | 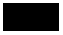 | 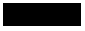 |
| 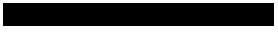 | 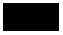 | 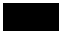 | 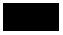 | 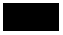 | 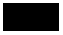 | 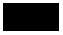 | 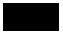 | 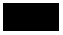 | 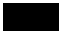 | 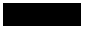 |
| 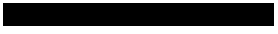 | 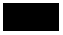 | 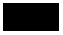 | 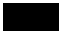 | 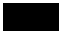 | 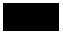 | 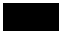 | 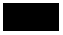 | 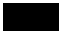 | 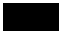 | 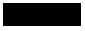 |
| 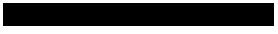 | 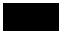 | 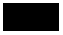 | 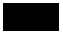 | 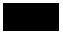 | 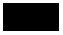 | 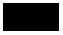 | 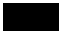 | 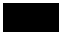 | 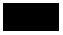 | 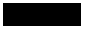 |
| 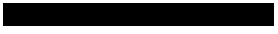 | 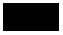 | 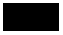 | 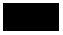 | 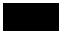 | 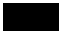 | 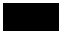 | 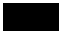 | 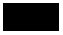 | 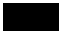 | 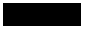 |
| 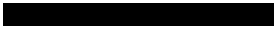 | 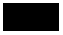 | 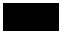 | 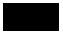 | 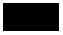 | 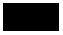 | 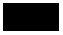 | 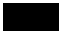 | 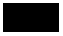 | 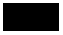 | 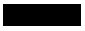 |
| 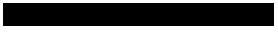 | 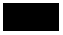 | 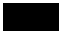 | 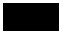 | 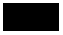 | 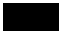 | 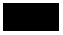 | 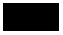 | 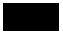 | 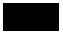 | 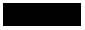 |
| 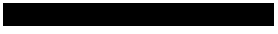 | 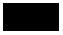 | 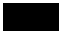 | 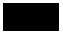 | 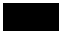 | 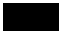 | 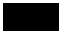 | 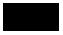 | 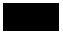 | 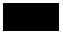 | 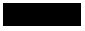 |
| 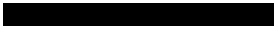 | 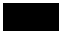 | 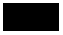 | 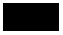 | 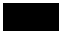 | 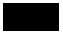 | 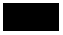 | 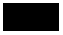 | 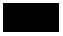 | 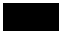 | 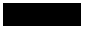 |
| 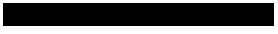 | 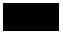 | 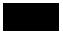 | 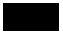 | 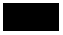 | 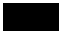 | 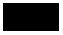 | 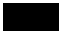 | 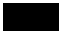 | 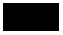 | 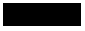 |
| 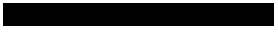 | 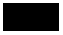 | 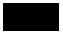 | 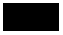 | 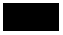 | 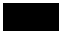 | 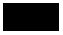 | 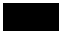 | 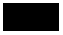 | 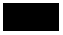 | 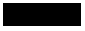 |

TABLE 10
Binding of the sera called SARS-yellow, SARS-green, 1a,
1b, 2, 6, 37, 62 and London to looped/cyclic peptides of protein X3
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| MFHLVDFQVTIAEIL | 0.8 | 0.6 | 0.8 | 0.6 | 1.0 | 0.8 | 0.3 | 0.8 | 0.6 | 999 |
| FHLVDFQVTIAEILI | 0.8 | 0.4 | 0.8 | 0.5 | 0.7 | 0.7 | 0.2 | 0.8 | 0.3 | 1000 |
| HLVDFQVTIAEILII | 0.7 | 0.4 | 0.6 | 0.4 | 0.6 | 0.7 | 0.2 | 0.7 | 0.2 | 1001 |
| LVDFQVTIAEILIII | 0.7 | 0.4 | 0.6 | 0.3 | 0.5 | 0.6 | 0.2 | 0.6 | 0.2 | 1002 |
| VDFQVTIAEILIIIM | 0.7 | 0.4 | 0.6 | 0.3 | 0.6 | 0.6 | 0.2 | 0.8 | 0.3 | 1003 |
| DFQVTIAEILIIIMR | 0.7 | 0.4 | 0.7 | 0.4 | 0.7 | 0.5 | 0.2 | 0.7 | 0.2 | 1004 |
| FQVTIAEILIIIMRT | 0.5 | 0.2 | 0.5 | 0.3 | 0.6 | 0.4 | 0.2 | 0.6 | 0.2 | 1005 |
| QVTIAEILIIIMRTF | 0.7 | 0.3 | 0.5 | 0.5 | 0.6 | 0.4 | 0.2 | 0.7 | 0.2 | 1006 |
| VTIAEILIIIMRTFR | 0.7 | 0.4 | 0.7 | 0.7 | 0.6 | 0.5 | 0.3 | 0.6 | 0.2 | 1007 |
| TIAEILIIIMRTFRI | 0.7 | 0.4 | 0.6 | 0.4 | 0.3 | 0.4 | 0.2 | 0.5 | 0.4 | 1008 |
| IAEILIIIMRTFRIA | 0.7 | 0.3 | 0.5 | 0.7 | 0.4 | 0.4 | 0.8 | 0.6 | 0.2 | 1009 |
| 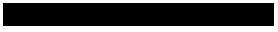 | 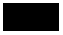 | 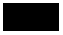 | 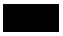 | 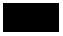 | 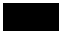 | 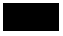 | 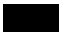 | 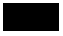 | 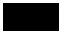 | 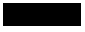 |
| 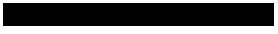 | 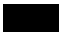 | 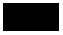 | 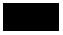 | 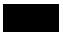 | 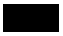 | 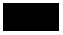 | 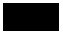 | 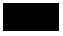 | 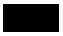 | 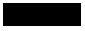 |
| 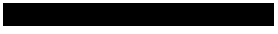 | 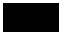 | 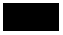 | 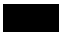 | 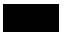 | 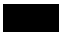 | 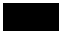 | 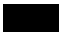 | 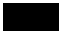 | 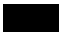 | 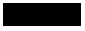 |
| 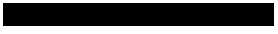 | 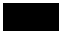 | 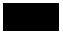 | 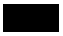 | 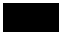 | 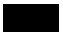 | 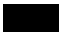 | 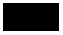 | 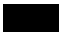 | 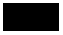 | 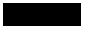 |
| 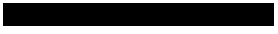 | 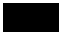 | 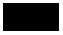 | 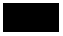 | 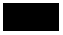 | 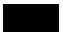 | 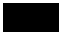 | 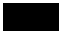 | 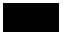 | 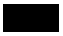 | 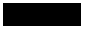 |
| 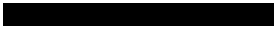 | 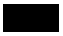 | 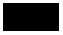 | 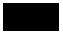 | 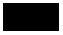 | 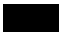 | 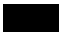 | 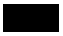 | 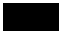 | 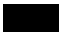 | 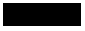 |
| 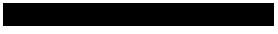 | 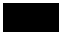 | 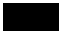 | 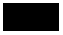 | 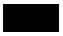 | 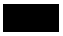 | 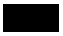 | 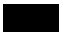 | 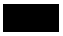 | 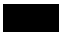 | 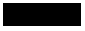 |
| 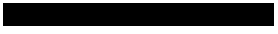 | 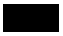 | 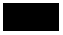 | 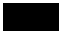 | 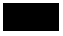 | 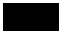 | 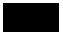 | 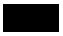 | 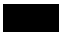 | 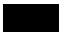 | 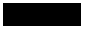 |
| 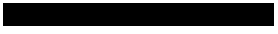 | 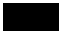 | 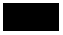 | 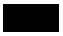 | 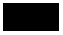 | 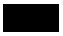 | 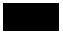 | 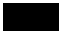 | 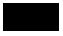 | 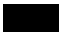 | 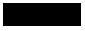 |
| TFRIAIWNLDVIISS | 0.7 | 0.5 | 0.6 | 0.3 | 0.6 | 0.7 | 0.2 | 0.5 | 0.2 | 1010 |
| FRIAIWNLDVIISSI | 0.7 | 0.5 | 0.7 | 0.3 | 0.6 | 0.6 | 0.2 | 0.8 | 0.2 | 1011 |
| RIAIWNLDVIISSIV | 0.8 | 0.5 | 0.8 | 0.4 | 0.6 | 0.7 | 0.2 | 1.0 | 0.3 | 1012 |
| IAIWNLDVIISSIVR | 0.7 | 0.3 | 0.6 | 0.4 | 0.6 | 0.5 | 0.2 | 0.6 | 0.0 | 1013 |
| AIWNLDVIISSIVRQ | 0.5 | 0.5 | 0.4 | 0.4 | 0.6 | 0.5 | 0.2 | 0.6 | 0.4 | 1014 |
| IWNLDVIISSIVRQL | 0.5 | 0.4 | 0.5 | 0.6 | 0.5 | 0.4 | 0.2 | 0.6 | 0.3 | 1015 |
| WNLDVIISSIVRQLF | 0.5 | 0.3 | 0.4 | 0.4 | 0.4 | 0.4 | 0.2 | 0.5 | 0.2 | 1016 |
| NLDVIISSIVRQLFK | 0.5 | 0.4 | 0.5 | 0.8 | 0.5 | 0.4 | 1.8 | 0.6 | 0.2 | 1017 |
| LDVIISSIVRQLFKP | 0.6 | 0.3 | 0.5 | 0.4 | 0.5 | 0.5 | 0.2 | 0.3 | 0.2 | 1018 |
| DVIISSIVRQLFKPL | 0.6 | 0.2 | 0.5 | 0.6 | 0.5 | 0.4 | 0.6 | 0.5 | 0.4 | 1019 |
| 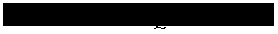 | 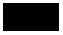 | 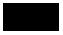 | 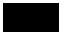 | 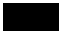 | 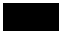 | 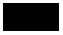 | 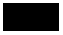 | 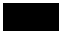 | 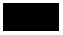 | 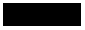 |
| 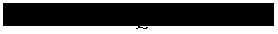 | 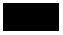 | 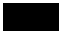 | 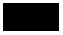 | 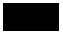 | 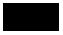 | 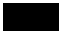 | 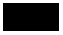 | 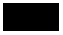 | 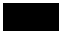 | 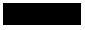 |
| 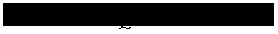 | 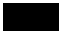 | 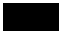 | 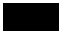 | 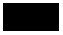 | 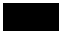 | 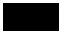 | 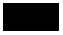 | 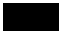 | 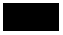 | 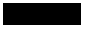 |
| 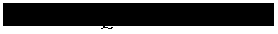 | 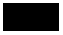 | 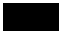 | 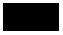 | 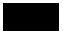 | 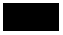 | 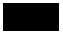 | 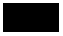 | 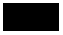 | 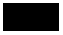 | 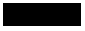 |
| 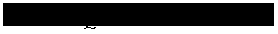 | 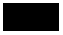 | 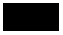 | 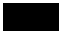 | 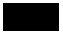 | 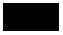 | 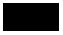 | 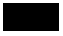 | 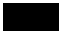 | 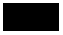 | 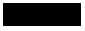 |
| 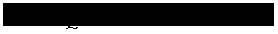 | 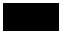 | 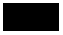 | 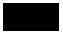 | 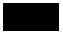 | 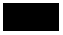 | 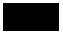 | 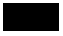 | 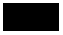 | 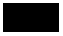 | 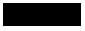 |
| 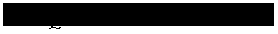 | 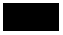 | 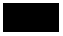 | 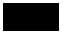 | 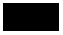 | 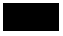 | 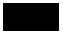 | 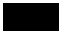 | 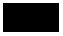 | 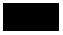 | 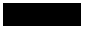 |
| 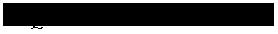 | 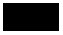 | 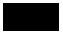 | 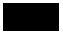 | 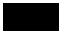 | 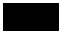 | 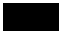 | 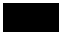 | 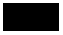 | 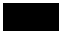 | 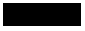 |
| 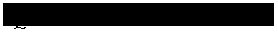 | 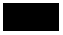 | 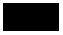 | 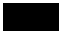 | 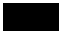 | 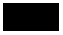 | 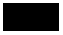 | 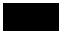 | 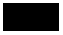 | 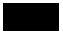 | 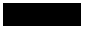 |
| 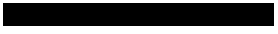 | 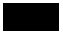 | 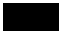 | 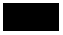 | 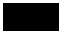 | 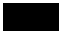 | 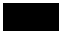 | 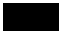 | 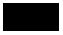 | 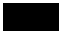 | 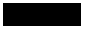 |
| 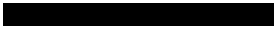 | 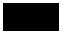 | 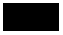 | 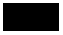 | 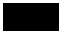 | 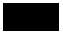 | 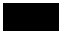 | 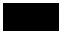 | 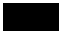 | 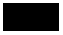 | 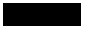 |
| 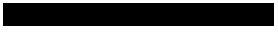 | 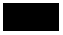 | 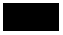 | 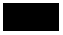 | 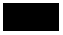 | 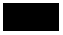 | 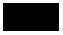 | 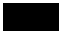 | 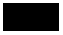 | 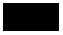 | 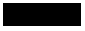 |
| 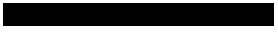 | 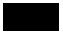 | 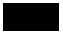 | 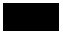 | 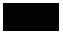 | 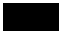 | 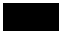 | 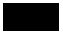 | 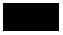 | 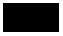 | 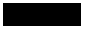 |
| 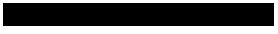 | 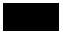 | 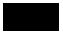 | 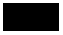 | 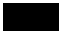 | 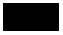 | 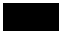 | 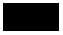 | 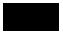 | 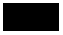 | 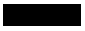 |
| 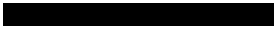 | 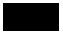 | 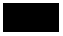 | 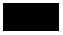 | 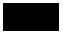 | 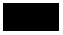 | 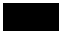 | 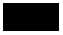 | 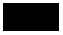 | 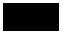 | 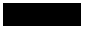 |
| 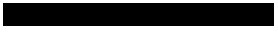 | 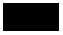 | 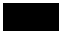 | 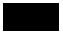 | 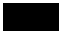 | 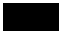 | 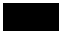 | 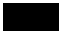 | 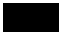 | 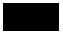 | 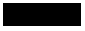 |
| 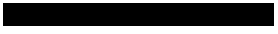 | 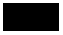 | 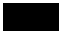 | 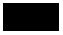 | 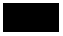 | 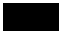 | 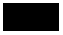 | 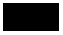 | 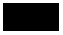 | 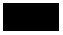 | 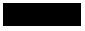 |
| 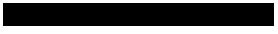 | 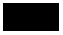 | 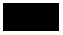 | 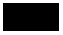 | 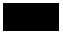 | 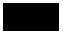 | 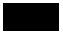 | 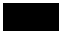 | 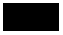 | 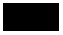 | 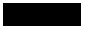 |
| 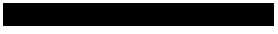 | 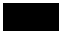 | 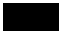 | 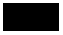 | 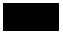 | 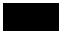 | 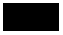 | 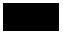 | 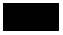 | 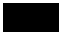 | 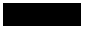 |

TABLE 11
Binding of the sera called SARS-yellow, SARS-green, 1a,
1b, 2, 6, 37, 62 and London to linear peptides of protein X4 of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MKIILFLTLIVFTSC | 0.6 | 0.5 | 0.4 | 0.4 | 0.7 | 0.7 | 0.5 | 0.4 | 0.7 | 1020 |
| KIILFLTLIVFTSCE | 0.8 | 0.9 | 0.7 | 0.7 | 1.1 | 0.9 | 0.9 | 0.7 | 0.8 | 1021 |
| IILFLTLIVFTSCEL | 0.8 | 0.8 | 0.6 | 0.5 | 0.9 | 0.8 | 0.7 | 0.6 | 0.7 | 1022 |
| ILFLTLIVFTSCELY | 0.7 | 0.6 | 0.5 | 0.5 | 0.7 | 0.7 | 0.4 | 0.6 | 0.6 | 1023 |
| LFLTLIVFTSCELYH | 0.7 | 0.7 | 0.5 | 0.5 | 0.7 | 0.7 | 0.6 | 0.7 | 0.6 | 1024 |
| FLTLIVFTSCELYHY | 0.7 | 0.6 | 0.5 | 0.5 | 0.7 | 0.7 | 0.6 | 0.6 | 0.6 | 1025 |
| LTLIVFTSCELYHYQ | 0.7 | 0.6 | 0.5 | 0.5 | 0.7 | 0.8 | 0.4 | 0.7 | 0.7 | 1026 |
| TLIVFTSCELYHYQE | 0.8 | 0.8 | 0.5 | 0.6 | 0.9 | 0.9 | 0.6 | 1.1 | 0.8 | 1027 |
| LIVFTSCELYHYQEC | 0.8 | 0.8 | 0.6 | 0.6 | 1.0 | 1.0 | 0.6 | 1.1 | 0.8 | 1028 |
| IVFTSCELYHYQECV | 0.8 | 0.9 | 0.6 | 0.8 | 1.0 | 1.0 | 0.4 | 0.9 | 0.8 | 1029 |
| VFTSCELYHYQECVR | 0.8 | 0.6 | 0.4 | 0.7 | 0.8 | 0.8 | 0.3 | 0.7 | 0.7 | 1030 |
| FTSCELYHYQECVRG | 0.8 | 0.7 | 0.5 | 0.9 | 0.8 | 0.8 | 0.3 | 0.5 | 0.9 | 1031 |
| TSCELYHYQECVRGT | 0.7 | 0.6 | 0.3 | 0.7 | 0.7 | 0.8 | 0.3 | 0.5 | 0.7 | 1032 |
| SCELYHYQECVRGTT | 0.8 | 0.6 | 0.4 | 0.5 | 0.7 | 0.6 | 0.2 | 0.5 | 0.8 | 1033 |
| CELYHYQECVRGTTV | 0.8 | 0.7 | 0.5 | 0.6 | 0.9 | 0.8 | 0.6 | 0.7 | 0.9 | 1034 |
| 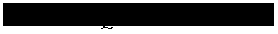 | 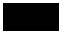 | 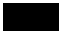 | 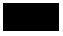 | 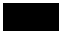 | 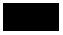 | 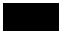 | 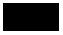 | 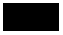 | 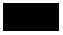 | 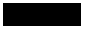 |
| 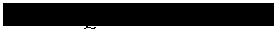 | 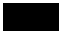 | 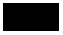 | 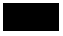 | 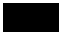 | 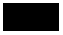 | 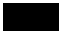 | 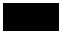 | 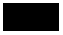 | 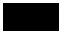 | 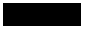 |
| 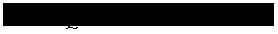 | 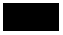 | 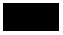 | 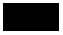 | 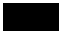 | 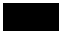 | 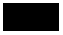 | 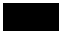 | 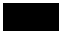 | 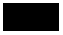 | 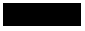 |
| 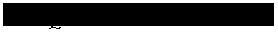 | 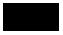 | 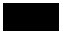 | 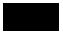 | 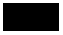 | 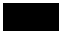 | 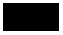 | 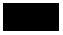 | 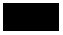 | 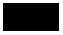 | 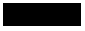 |
| 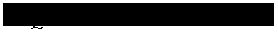 | 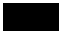 | 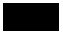 | 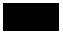 | 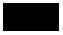 | 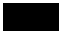 | 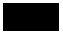 | 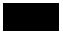 | 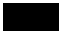 | 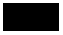 | 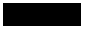 |
| 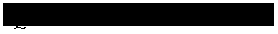 | 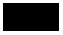 | 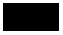 | 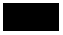 | 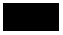 | 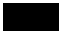 | 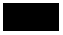 | 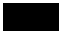 | 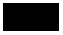 | 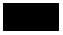 | 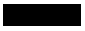 |
| 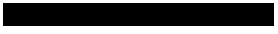 | 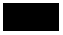 | 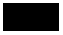 | 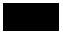 | 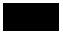 | 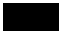 | 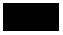 | 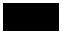 | 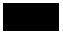 | 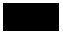 | 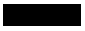 |
| 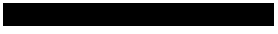 | 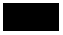 | 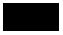 | 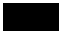 | 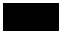 | 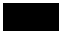 | 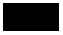 | 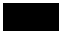 | 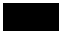 | 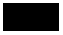 | 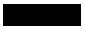 |
| 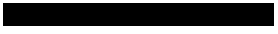 | 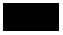 | 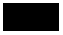 | 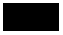 | 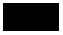 | 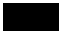 | 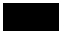 | 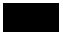 | 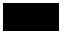 | 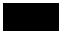 | 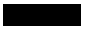 |
| 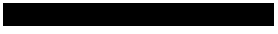 | 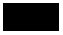 | 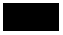 | 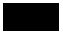 | 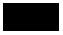 | 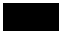 | 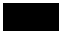 | 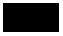 | 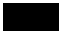 | 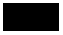 | 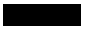 |
| 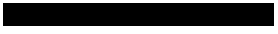 | 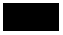 | 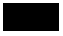 | 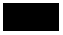 | 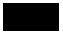 | 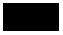 | 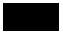 | 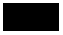 | 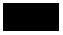 | 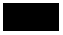 | 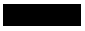 |
| 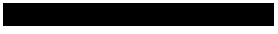 | 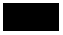 | 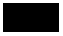 | 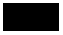 | 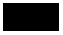 | 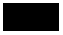 | 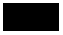 | 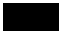 | 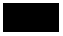 | 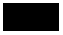 | 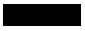 |
| 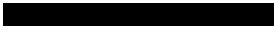 | 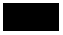 | 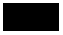 | 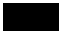 | 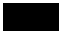 | 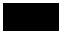 | 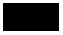 | 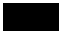 | 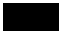 | 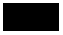 | 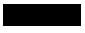 |
| VLLKEPCPSGTYEGN | 0.8 | 0.7 | 0.4 | 0.7 | 0.7 | 0.8 | 0.1 | 0.6 | 0.8 | 1035 |
| LLKEPCPSGTYEGNS | 0.8 | 0.6 | 0.3 | 0.6 | 0.6 | 0.8 | 0.2 | 0.5 | 0.8 | 1036 |
| LKEPCPSGTYEGNSP | 0.7 | 0.4 | 0.4 | 0.3 | 0.6 | 0.7 | 0.2 | 0.5 | 0.9 | 1037 |
| KEPCPSGTYEGNSPF | 0.7 | 0.6 | 0.4 | 0.4 | 0.7 | 0.8 | 0.7 | 0.6 | 0.8 | 1038 |
| EPCPSGTYEGNSPFH | 0.7 | 0.5 | 0.4 | 0.5 | 0.6 | 0.7 | 0.9 | 0.6 | 0.7 | 1039 |
| PCPSGTYEGNSPFHP | 0.7 | 0.5 | 0.4 | 0.5 | 0.7 | 0.7 | 0.7 | 0.5 | 0.7 | 1040 |
| 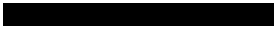 | 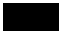 | 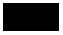 | 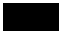 | 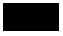 | 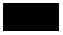 | 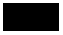 | 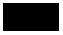 | 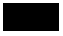 | 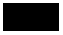 | 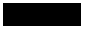 |
| 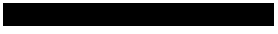 | 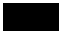 | 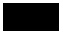 | 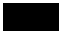 | 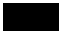 | 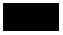 | 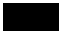 | 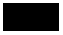 | 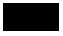 | 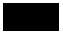 | 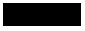 |
| 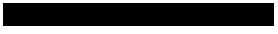 | 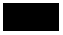 | 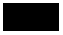 | 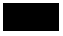 | 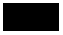 | 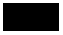 | 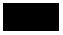 | 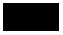 | 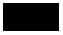 | 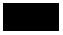 | 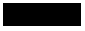 |
| 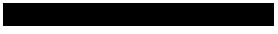 | 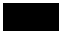 | 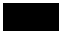 | 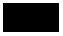 | 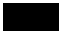 | 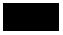 | 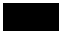 | 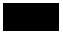 | 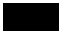 | 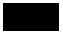 | 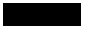 |
| 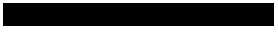 | 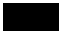 | 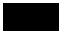 | 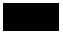 | 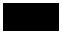 | 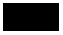 | 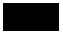 | 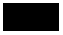 | 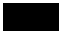 | 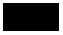 | 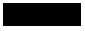 |
| 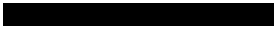 | 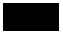 | 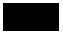 | 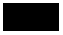 | 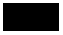 | 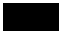 | 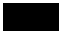 | 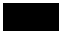 | 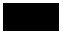 | 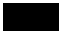 | 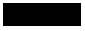 |
| 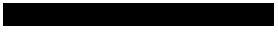 | 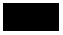 | 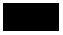 | 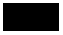 | 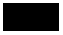 | 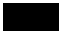 | 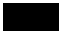 | 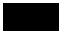 | 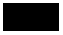 | 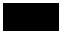 | 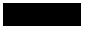 |
| 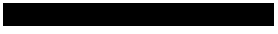 | 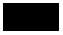 | 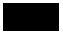 | 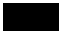 | 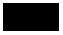 | 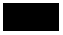 | 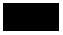 | 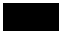 | 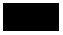 | 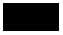 | 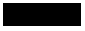 |
| 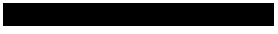 | 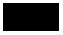 | 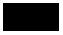 | 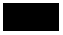 | 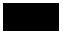 | 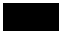 | 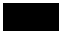 | 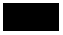 | 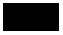 | 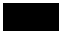 | 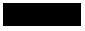 |
| 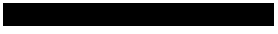 | 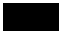 | 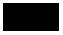 | 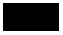 | 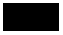 | 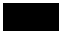 | 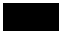 | 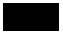 | 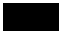 | 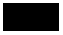 | 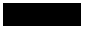 |
| 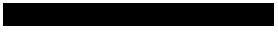 | 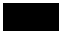 | 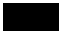 | 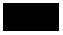 | 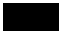 | 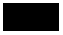 | 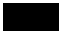 | 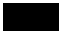 | 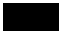 | 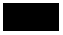 | 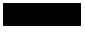 |
| 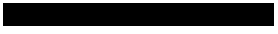 | 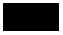 | 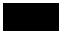 | 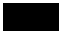 | 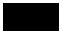 | 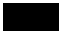 | 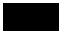 | 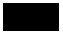 | 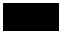 | 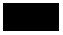 | 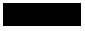 |
| 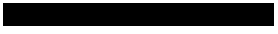 | 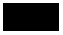 | 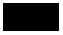 | 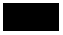 | 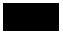 | 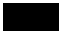 | 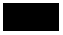 | 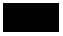 | 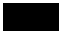 | 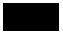 | 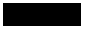 |
| 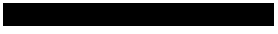 | 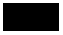 | 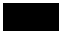 | 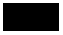 | 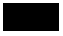 | 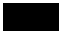 | 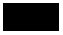 | 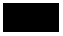 | 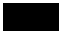 | 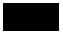 | 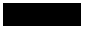 |
| 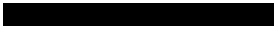 | 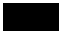 | 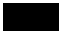 | 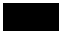 | 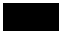 | 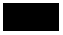 | 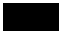 | 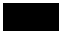 | 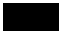 | 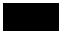 | 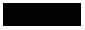 |
| 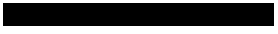 | 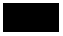 | 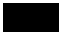 | 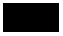 | 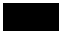 | 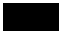 | 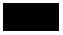 | 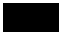 | 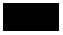 | 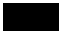 | 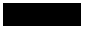 |
| 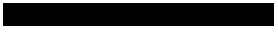 | 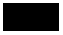 | 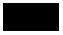 | 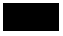 | 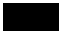 | 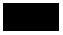 | 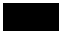 | 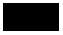 | 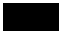 | 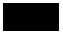 | 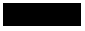 |
| NKFALTCTSTHFAFA | 0.6 | 0.5 | 0.4 | 0.3 | 0.7 | 0.7 | 0.5 | 0.4 | 0.7 | 1041 |
| KFALTCTSTHFAFAC | 0.8 | 0.6 | 0.5 | 0.8 | 0.8 | 0.8 | 0.6 | 0.4 | 0.8 | 1042 |
| FALTCTSTHFAFACA | 0.7 | 0.5 | 0.4 | 0.5 | 0.7 | 0.7 | 0.6 | 0.5 | 0.7 | 1043 |
| ALTCTSTHFAFACAD | 0.8 | 0.8 | 0.5 | 0.6 | 0.9 | 0.9 | 0.7 | 0.8 | 0.8 | 1044 |
| LTCTSTHFAFACADG | 0.8 | 0.8 | 0.5 | 0.6 | 0.8 | 0.8 | 0.5 | 0.8 | 0.8 | 1045 |
| TCTSTHFAFACADGT | 0.8 | 0.7 | 0.5 | 0.7 | 0.7 | 0.9 | 0.6 | 0.6 | 0.8 | 1046 |
| CTSTHFAFACADGTR | 0.7 | 0.7 | 0.5 | 0.6 | 0.7 | 0.8 | 0.4 | 0.6 | 0.7 | 1047 |
| TSTHFAFACADGTRH | 0.7 | 0.6 | 0.5 | 0.7 | 0.7 | 0.8 | 0.2 | 0.6 | 0.8 | 1048 |
| STHFAFACADGTRHT | 0.7 | 0.6 | 0.5 | 0.7 | 0.7 | 0.8 | 0.3 | 0.6 | 0.8 | 1049 |
| THFAFACADGTRHTY | 0.7 | 0.5 | 0.5 | 0.6 | 0.6 | 0.7 | 0.2 | 0.5 | 0.7 | 1050 |
| HFAFACADGTRHTYQ | 0.7 | 0.5 | 0.4 | 0.6 | 0.6 | 0.6 | 0.1 | 0.6 | 0.6 | 1051 |
| FAFACADGTRHTYQL | 0.7 | 0.5 | 0.3 | 0.5 | 0.6 | 0.7 | 0.2 | 0.4 | 0.6 | 1052 |
| AFACADGTRHTYQLR | 0.6 | 0.4 | 0.4 | 0.5 | 0.6 | 0.7 | 0.2 | 0.4 | 0.6 | 1053 |
| FACADGTRHTYQLRA | 0.7 | 0.4 | 0.4 | 0.4 | 0.6 | 0.7 | 0.2 | 0.5 | 0.8 | 531 |
| ACADGTRHTYQLRAR | 0.7 | 0.6 | 0.5 | 0.6 | 0.7 | 0.8 | 0.5 | 0.7 | 0.7 | 532 |
| CADGTRHTYQLRARS | 0.6 | 0.5 | 0.5 | 0.6 | 0.7 | 0.7 | 0.7 | 0.6 | 0.8 | 533 |
| ADGTRHTYQLRARSV | 0.7 | 0.6 | 0.6 | 0.5 | 0.7 | 0.7 | 0.7 | 0.6 | 0.7 | 534 |
| DGTRHTYQLRARSVS | 0.6 | 0.5 | 0.8 | 0.8 | 0.8 | 0.7 | 0.5 | 0.4 | 0.7 | 535 |
| GTRHTYQLRARSVSP | 0.7 | 0.6 | 0.8 | 0.6 | 0.8 | 0.8 | 0.8 | 0.6 | 0.8 | 536 |
| TRHTYQLRARSVSPK | 0.8 | 0.7 | 0.7 | 1.3 | 1.0 | 0.9 | 0.9 | 0.6 | 0.8 | 537 |
| RHTYQLRARSVSPKL | 0.8 | 0.6 | 0.6 | 1.1 | 0.9 | 0.8 | 0.6 | 0.7 | 0.8 | 538 |
| HTYQLRARSVSPKLF | 0.7 | 0.8 | 0.6 | 1.0 | 1.0 | 0.9 | 0.5 | 0.7 | 0.8 | 539 |
| TYQLRARSVSPKLFI | 0.8 | 0.6 | 0.7 | 0.9 | 0.8 | 0.9 | 0.6 | 0.7 | 0.9 | 540 |
| YQLRARSVSPKLFIR | 0.7 | 0.6 | 0.6 | 0.7 | 0.8 | 0.7 | 0.6 | 0.7 | 0.8 | 541 |
| QLRARSVSPKLFIRQ | 0.7 | 0.6 | 0.6 | 0.9 | 0.7 | 0.7 | 0.3 | 0.6 | 0.8 | 542 |
| LRARSVSPKLFIRQE | 0.7 | 0.6 | 0.5 | 0.8 | 0.7 | 0.8 | 0.3 | 0.6 | 0.8 | 543 |
| RARSVSPKLFIRQEE | 0.8 | 0.6 | 0.4 | 0.6 | 0.7 | 0.8 | 0.1 | 0.6 | 0.8 | 544 |
| ARSVSPKLFIRQEEV | 0.7 | 0.6 | 0.5 | 0.6 | 0.7 | 0.7 | 0.1 | 0.6 | 0.7 | 1054 |
| RSVSPKLFIRQEEVQ | 0.7 | 0.4 | 0.4 | 0.5 | 0.6 | 0.7 | 0.2 | 0.5 | 0.6 | 1055 |
| SVSPKLFIRQEEVQQ | 0.7 | 0.5 | 0.3 | 0.5 | 0.7 | 0.7 | 0.3 | 0.4 | 0.6 | 1056 |
| VSPKLFIRQEEVQQE | 0.7 | 0.5 | 0.3 | 0.3 | 0.7 | 0.7 | 0.3 | 0.6 | 0.8 | 1057 |
| SPKLFIRQEEVQQEL | 0.7 | 0.5 | 0.3 | 0.4 | 0.7 | 0.7 | 0.5 | 0.8 | 0.7 | 1058 |
| PKLFIRQEEVQQELY | 0.7 | 0.6 | 0.4 | 0.5 | 0.8 | 0.8 | 0.8 | 0.7 | 0.8 | 10S9 |
| KLFIRQEEVQQELYS | 0.7 | 0.5 | 0.4 | 0.5 | 0.7 | 0.8 | 0.4 | 0.6 | 0.7 | 1060 |
| LFIRQEEVQQELYSP | 0.8 | 0.7 | 0.6 | 0.5 | 0.7 | 0.8 | 0.7 | 0.6 | 0.8 | 1061 |
| FTRQEEVQQELYSPL | 0.8 | 0.6 | 0.5 | 0.5 | 0.8 | 0.7 | 0.7 | 0.5 | 0.7 | 327 |
| IRQEEVQQELYSPLF | 0.7 | 0.6 | 0.5 | 0.5 | 0.8 | 0.8 | 0.7 | 0.8 | 0.7 | 328 |
| RQEEVQQELYSPLFL | 0.8 | 0.6 | 0.5 | 0.5 | 0.8 | 0.8 | 0.7 | 0.7 | 0.7 | 329 |
| QEEVQQELYSPLFLI | 0.8 | 0.9 | 0.6 | 0.6 | 0.8 | 0.8 | 0.8 | 1.0 | 0.8 | 330 |
| EEVQQELYSPLFLIV | 0.7 | 0.6 | 0.6 | 0.6 | 0.7 | 0.8 | 0.8 | 0.7 | 0.7 | 331 |
| EVQQELYSPLFLIVA | 0.7 | 0.6 | 0.4 | 0.9 | 0.8 | 0.7 | 0.6 | 0.8 | 1.0 | 332 |
| VQQELYSPLFLIVAA | 0.7 | 0.6 | 0.6 | 0.9 | 0.7 | 0.7 | 0.5 | 0.6 | 0.9 | 333 |
| QQELYSPLFLIVAAL | 0.7 | 0.5 | 0.5 | 0.7 | 0.7 | 0.6 | 0.6 | 0.6 | 0.8 | 1062 |
| QELYSPLFLIVAALV | 0.7 | 0.5 | 0.8 | 0.6 | 0.7 | 0.7 | 0.2 | 0.5 | 0.7 | 1063 |
| ELYSPLFLIVAALVF | 0.7 | 0.4 | 0.4 | 0.4 | 0.6 | 0.6 | 0.2 | 0.5 | 0.5 | 1064 |
| LYSPLFLIVAALVFL | 0.6 | 0.4 | 0.4 | 0.5 | 0.6 | 0.6 | 0.2 | 0.5 | 0.6 | 1065 |
| YSPLFLIVAALVFLI | 0.7 | 0.3 | 0.5 | 0.5 | 0.7 | 0.7 | 0.4 | 0.5 | 0.7 | 1066 |
| SPLFLIVAALVFLIL | 0.6 | 0.2 | 0.5 | 0.2 | 0.6 | 0.6 | 0.3 | 0.5 | 0.6 | 1067 |
| PLFLIVAALVFLILC | 0.5 | 0.4 | 0.3 | 0.3 | 0.6 | 0.7 | 0.4 | 0.5 | 0.6 | 1068 |
| LFLIVAALVFLILCF | 0.6 | 0.5 | 0.3 | 0.4 | 0.6 | 0.6 | 0.7 | 0.6 | 0.7 | 1069 |
| FLIVAALVFLILCFT | 0.6 | 0.5 | 0.4 | 0.5 | 0.7 | 0.7 | 0.7 | 0.5 | 0.6 | 1070 |
| LIVAALVFLILCFTI | 0.7 | 0.5 | 0.5 | 0.4 | 0.6 | 0.6 | 0.4 | 0.5 | 0.6 | 1071 |
| IVAALVFLILCFTIK | 0.6 | 0.6 | 0.4 | 0.5 | 0.7 | 0.7 | 0.6 | 0.5 | 0.7 | 1072 |
| VAALVFLILCFTTKR | 0.6 | 0.5 | 0.4 | 0.5 | 0.7 | 0.7 | 0.5 | 0.5 | 0.6 | 1073 |
| AALVFLILCFTIKRK | 0.7 | 0.8 | 0.6 | 0.8 | 0.8 | 0.8 | 0.7 | 0.7 | 0.8 | 1074 |
| ALVFLILCFTIKRKT | 0.7 | 0.6 | 0.7 | 0.7 | 0.7 | 0.8 | 0.4 | 0.7 | 0.8 | 1075 |
| LVFLILCFTIKRKTE | 0.7 | 0.6 | 0.5 | 0.7 | 0.7 | 0.8 | 0.6 | 0.7 | 0.8 | 1076 |

TABLE 12
Binding of the sera called SARS-yellow, SARS-green, 1a,
1b, 2, 6, 37, 62 and London to looped/cyclic peptides of protein X4
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MKIILFLTLIVFTSC | 0.6 | 0.0 | 0.5 | 0.5 | 0.6 | 0.5 | 0.6 | 0.3 | 0.2 | 1020 |
| KIILFLTLIVFTSCE | 0.9 | 0.2 | 0.8 | 0.6 | 0.9 | 0.9 | 0.8 | 0.7 | 0.4 | 1021 |
| IILFLTLIVFTSCEL | 0.7 | 0.1 | 0.6 | 0.5 | 0.7 | 0.7 | 0.6 | 0.4 | 0.2 | 1022 |
| ILFLTLIVFTSCELY | 0.7 | 0.4 | 0.7 | 0.6 | 0.7 | 0.7 | 0.6 | 0.4 | 0.3 | 1023 |
| LFLTLIVFTSCELYH | 0.9 | 0.8 | 0.8 | 0.7 | 0.9 | 0.9 | 0.6 | 0.7 | 0.3 | 1024 |
| FLTLIVFTSCELYHY | 0.8 | 1.7 | 0.8 | 0.5 | 0.7 | 0.7 | 0.7 | 0.3 | 0.3 | 1025 |
| LTLIVFTSCELYHYQ | 0.8 | 0.7 | 0.7 | 0.5 | 0.7 | 0.7 | 0.7 | 0.4 | 0.3 | 1026 |
| TLIVFTSCELYHYQE | 0.8 | 0.8 | 0.8 | 0.6 | 0.9 | 0.8 | 0.9 | 1.0 | 0.3 | 1027 |
| LIVFTSCELYHYQEC | 0.8 | 0.6 | 0.6 | 0.5 | 0.7 | 0.7 | 0.6 | 0.5 | 0.3 | 1028 |
| IVFTSCELYHYQECV | 0.9 | 0.8 | 0.8 | 0.6 | 1.0 | 0.8 | 0.6 | 0.8 | 0.3 | 1029 |
| VFTSCELYHYQECVR | 0.8 | 0.7 | 0.7 | 0.7 | 0.8 | 0.7 | 0.5 | 0.7 | 0.3 | 1030 |
| FTSGELYHYQECVRG | 0.8 | 0.7 | 0.6 | 0.6 | 0.8 | 0.7 | 0.5 | 0.6 | 0.3 | 1031 |
| TSCELYHYQECVRGT | 0.9 | 0.9 | 0.7 | 0.9 | 0.7 | 0.7 | 0.4 | 0.7 | 0.4 | 1032 |
| SCELYHYQECVRGTT | 0.5 | 0.4 | 0.5 | 0.5 | 0.6 | 0.5 | 0.2 | 0.2 | 0.3 | 1033 |
| CELYHYQECVRGTTV | 0.8 | 0.4 | 0.8 | 0.5 | 0.8 | 0.6 | 0.4 | 0.2 | 0.3 | 1034 |
| 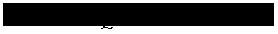 | 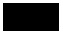 | 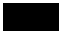 | 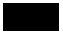 | 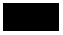 | 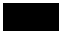 | 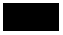 | 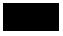 | 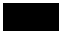 | 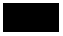 | 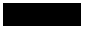 |
| 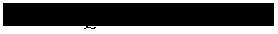 | 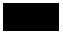 | 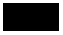 | 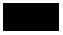 | 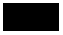 | 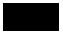 | 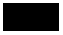 | 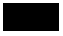 | 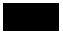 | 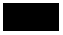 | 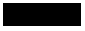 |
| 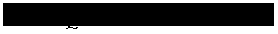 | 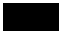 | 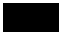 | 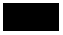 | 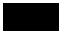 | 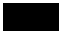 | 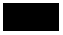 | 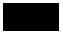 | 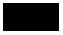 | 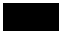 | 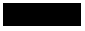 |
| 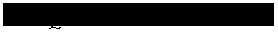 | 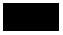 | 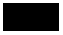 | 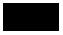 | 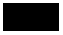 | 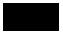 | 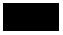 | 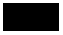 | 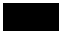 | 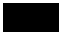 | 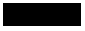 |
| 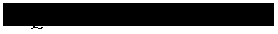 | 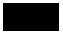 | 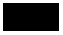 | 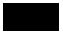 | 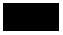 | 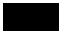 | 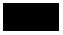 | 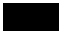 | 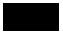 | 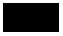 | 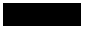 |
| 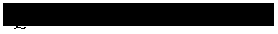 | 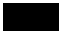 | 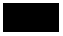 | 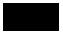 | 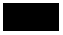 | 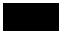 | 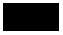 | 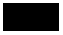 | 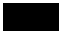 | 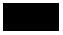 | 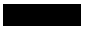 |
| 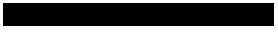 | 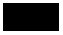 | 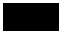 | 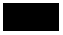 | 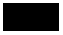 | 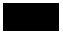 | 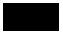 | 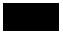 | 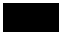 | 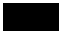 | 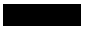 |
| 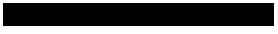 | 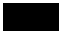 | 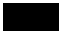 | 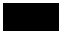 | 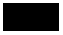 | 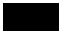 | 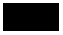 | 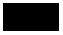 | 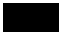 | 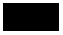 | 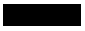 |
| 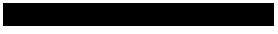 | 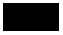 | 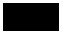 | 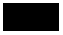 | 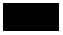 | 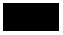 | 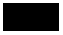 | 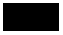 | 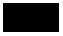 | 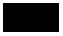 | 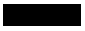 |
| 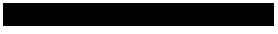 | 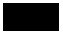 | 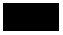 | 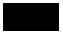 | 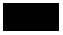 | 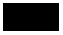 | 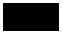 | 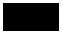 | 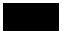 | 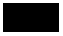 | 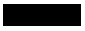 |
| 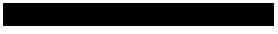 | 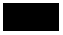 | 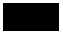 | 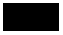 | 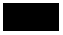 | 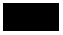 | 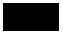 | 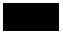 | 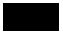 | 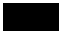 | 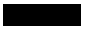 |
| 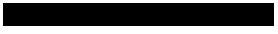 | 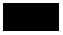 | 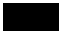 | 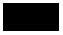 | 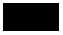 | 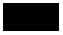 | 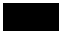 | 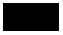 | 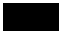 | 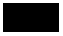 | 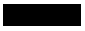 |
| 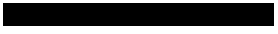 | 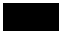 | 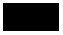 | 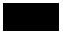 | 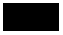 | 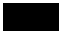 | 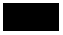 | 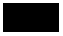 | 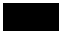 | 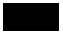 | 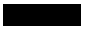 |
| VLLKEPCPSGTYEGN | 0.8 | 0.7 | 0.6 | 0.7 | 0.8 | 0.7 | 0.4 | 0.5 | 0.3 | 1035 |
| LLKEPCPSGTYEGNS | 0.8 | 0.6 | 0.6 | 0.7 | 0.7 | 0.5 | 0.2 | 0.7 | 0.3 | 1036 |
| LKEPCPSGTYEGNSP | 0.6 | 0.5 | 0.5 | 0.3 | 0.5 | 0.6 | 0.2 | 0.6 | 0.3 | 1037 |
| KEPCPSGTYEGNSPF | 0.8 | 0.6 | 0.6 | 0.6 | 0.7 | 0.6 | 0.3 | 0.4 | 0.3 | 1038 |
| EPCPSGTYEGNSPFH | 0.8 | 0.6 | 0.7 | 0.6 | 0.7 | 0.6 | 0.5 | 0.5 | 0.4 | 1039 |
| PCPSGTYEGNSPFHP | 0.7 | 0.1 | 0.7 | 0.5 | 0.7 | 0.7 | 0.4 | 0.4 | 0.3 | 1040 |
| 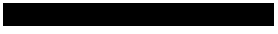 | 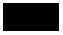 | 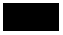 | 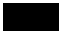 | 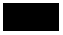 | 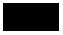 | 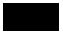 | 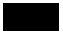 | 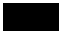 | 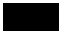 | 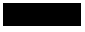 |
| 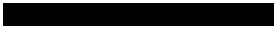 | 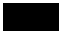 | 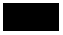 | 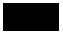 | 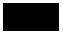 | 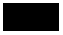 | 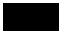 | 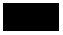 | 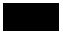 | 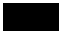 | 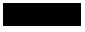 |
| 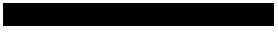 | 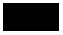 | 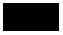 | 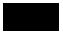 | 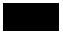 | 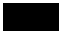 | 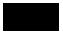 | 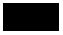 | 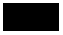 | 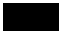 | 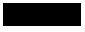 |
| 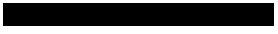 | 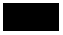 | 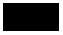 | 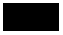 | 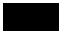 | 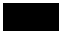 | 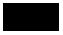 | 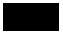 | 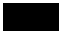 | 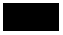 | 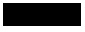 |
| 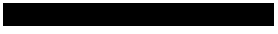 | 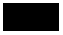 | 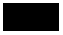 | 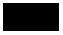 | 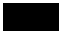 | 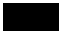 | 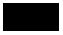 | 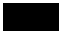 | 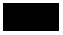 | 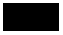 | 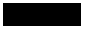 |
| 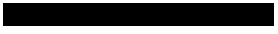 | 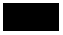 | 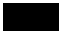 | 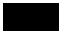 | 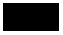 | 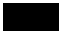 | 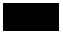 | 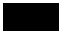 | 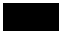 | 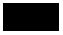 | 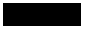 |
| 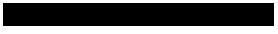 | 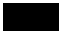 | 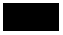 | 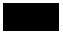 | 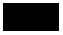 | 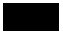 | 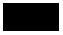 | 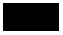 | 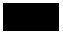 | 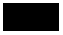 | 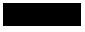 |
| 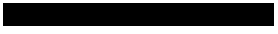 | 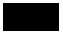 | 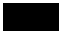 | 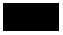 | 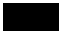 | 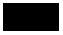 | 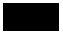 | 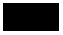 | 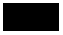 | 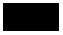 | 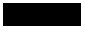 |
| 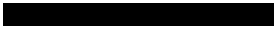 | 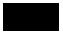 | 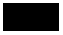 | 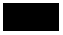 | 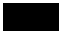 | 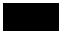 | 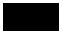 | 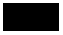 | 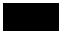 | 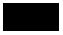 | 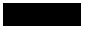 |
| 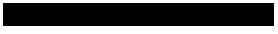 | 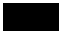 | 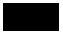 | 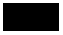 | 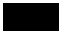 | 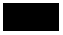 | 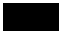 | 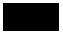 | 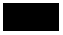 | 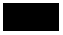 | 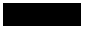 |
| 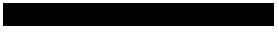 | 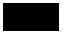 | 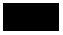 | 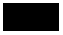 | 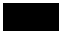 | 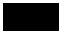 | 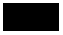 | 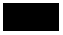 | 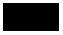 | 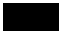 | 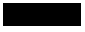 |
| 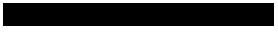 | 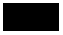 | 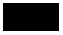 | 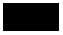 | 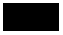 | 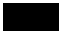 | 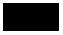 | 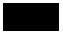 | 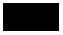 | 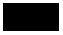 | 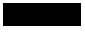 |
| 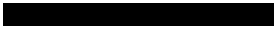 | 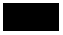 | 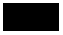 | 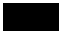 | 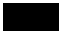 | 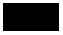 | 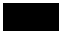 | 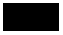 | 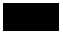 | 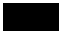 | 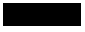 |
| 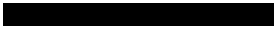 | 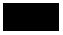 | 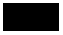 | 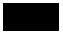 | 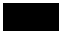 | 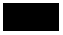 | 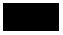 | 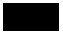 | 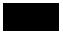 | 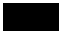 | 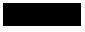 |
| 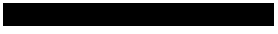 | 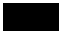 | 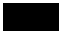 | 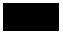 | 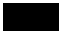 | 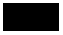 | 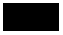 | 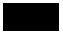 | 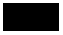 | 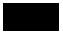 | 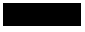 |
| 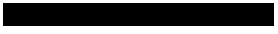 | 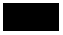 | 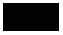 | 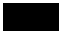 | 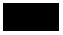 | 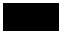 | 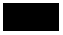 | 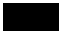 | 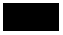 | 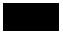 | 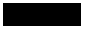 |
| 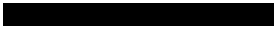 | 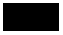 | 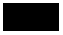 | 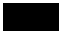 | 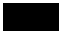 | 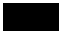 | 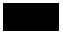 | 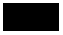 | 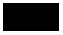 | 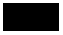 | 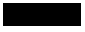 |
| NKFALTCTSTHFAFA | 0.9 | 0.4 | 0.8 | 0.6 | 0.7 | 0.9 | 0.7 | 0.8 | 0.3 | 1041 |
| KFALTCTSTHFAFAC | 1.0 | 0.7 | 0.9 | 0.7 | 0.7 | 0.9 | 0.8 | 0.8 | 0.4 | 1042 |
| FALTCTSTHFAFACA | 0.8 | 0.5 | 0.7 | 0.5 | 0.6 | 0.7 | 0.7 | 0.5 | 0.3 | 1043 |
| ALTCTSTHFAFACAD | 0.8 | 0.7 | 0.7 | 0.7 | 0.9 | 0.9 | 0.8 | 0.8 | 0.3 | 1044 |
| LTCTSTHFAFACADG | 0.6 | 0.3 | 0.6 | 0.5 | 0.5 | 0.6 | 0.4 | 0.4 | 0.3 | 1045 |
| TCTSTHFAFACADGT | 0.8 | 0.6 | 0.8 | 0.6 | 0.7 | 0.8 | 0.8 | 0.5 | 0.3 | 1046 |
| CTSTHFAFACADGTR | 0.7 | 0.4 | 0.6 | 0.6 | 0.7 | 0.7 | 1.0 | 0.4 | 0.3 | 1047 |
| TSTHFAFACADGTRH | 0.8 | 0.7 | 0.6 | 0.7 | 0.7 | 0.8 | 0.7 | 0.6 | 0.4 | 1048 |
| STHFAFACADGTRHT | 0.7 | 0.5 | 0.6 | 0.5 | 0.6 | 0.5 | 0.6 | 0.5 | 0.3 | 1049 |
| THFAFACADGTRHTY | 0.7 | 0.5 | 0.5 | 0.5 | 0.6 | 0.6 | 0.5 | 0.4 | 0.3 | 1050 |
| HFAFACADGTRHTYQ | 0.6 | 0.5 | 0.6 | 0.6 | 0.6 | 0.5 | 0.4 | 0.2 | 0.2 | 1051 |
| FAFACADGTRHTYQL | 0.8 | 0.5 | 0.9 | 0.5 | 0.6 | 0.5 | 0.4 | 0.5 | 0.2 | 1052 |
| AFACADGTRHTYQLR | 0.6 | 0.4 | 0.8 | 0.9 | 0.6 | 0.5 | 0.4 | 0.5 | 0.4 | 1053 |
| FACADGTRHTYQLRA | 0.7 | 0.6 | 0.6 | 0.3 | 0.7 | 0.7 | 0.3 | 0.8 | 0.4 | 531 |
| ACADGTRHTYQLRAR | 0.7 | 0.3 | 0.6 | 0.6 | 0.7 | 0.6 | 0.5 | 0.5 | 0.3 | 532 |
| CADGTRHTYQLRARS | 0.7 | 0.4 | 0.8 | 0.6 | 0.7 | 0.6 | 0.6 | 0.0 | 0.3 | 533 |
| ADGTRHTYQLRARSV | 0.7 | 0.4 | 0.6 | 0.7 | 0.7 | 0.6 | 0.6 | 0.5 | 0.3 | 534 |
| DGTRHTYQLRARSVS | 0.8 | 0.5 | 0.8 | 0.8 | 0.7 | 0.7 | 0.7 | 0.6 | 0.4 | 535 |
| GTRHTYQLRARSVSP | 0.8 | 0.5 | 1.0 | 0.6 | 0.7 | 0.8 | 0.7 | 0.5 | 0.3 | 536 |
| TRHTYQLRARSVSPK | 0.8 | 0.4 | 0.8 | 0.9 | 0.7 | 0.6 | 0.7 | 0.7 | 0.4 | 537 |
| RHTYQLRARSVSPKL | 0.8 | 0.5 | 0.7 | 0.8 | 0.7 | 0.6 | 0.7 | 0.4 | 0.3 | 538 |
| HTYQLRARSVSPKLF | 0.8 | 0.5 | 0.8 | 0.8 | 0.6 | 0.6 | 0.8 | 0.4 | 0.3 | 539 |
| TYQLRARSVSPKLFI | 0.9 | 0.7 | 0.8 | 1.3 | 0.6 | 0.8 | 0.9 | 0.2 | 1.4 | 540 |
| YQLRARSVSPKLFIR | 0.7 | 0.4 | 0.6 | 1.2 | 0.7 | 0.6 | 0.9 | 0.4 | 1.4 | 541 |
| QLRARSVSPKLFIRQ | 0.7 | 0.6 | 0.8 | 0.9 | 0.6 | 0.6 | 0.8 | 0.4 | 0.4 | 542 |
| LRARSVSPKLFIRQE | 0.8 | 0.6 | 0.6 | 0.8 | 0.7 | 0.7 | 0.7 | 0.6 | 0.4 | 543 |
| RARSVSPKLFIRQEE | 0.8 | 0.7 | 0.6 | 0.8 | 0.7 | 0.7 | 0.7 | 0.7 | 0.5 | 544 |
| ARSVSPKLFIRQEEV | 0.8 | 0.5 | 0.7 | 0.5 | 0.7 | 0.6 | 0.5 | 0.6 | 0.3 | 1054 |
| RSVSPKLFIRQEEVQ | 0.6 | 0.5 | 0.5 | 0.5 | 0.7 | 0.6 | 0.5 | 0.4 | 0.3 | 1055 |
| SVSPKLFIRQEEVQQ | 0.7 | 0.6 | 0.6 | 0.4 | 0.7 | 0.6 | 0.4 | 0.7 | 0.3 | 1056 |
| VSPKLFIRQEEVQQE | 0.6 | 1.0 | 0.8 | 0.4 | 0.9 | 0.8 | 0.5 | 1.2 | 0.6 | 1057 |
| SPKLFIRQEEVQQEL | 0.8 | 0.6 | 0.7 | 0.6 | 0.8 | 0.6 | 0.5 | 0.2 | 0.4 | 1058 |
| PKLFIRQEEVQQELY | 0.8 | 0.6 | 0.5 | 0.5 | 0.7 | 0.6 | 0.4 | 0.5 | 0.4 | 1059 |
| KLFIRQEEVQQELYS | 0.7 | 0.6 | 0.6 | 0.4 | 0.7 | 0.6 | 0.6 | 0.8 | 0.3 | 1060 |
| LFIRQEEVQQELYSP | 0.7 | 0.5 | 0.7 | 0 5 | 0.7 | 0.8 | 0.7 | 0.8 | 0.3 | 1061 |
| 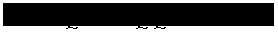 | 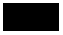 | 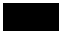 | 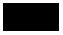 | 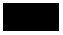 | 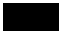 | 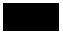 | 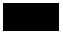 | 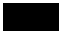 | 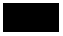 | 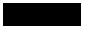 |
| 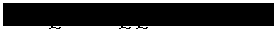 | 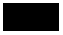 | 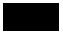 | 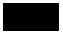 | 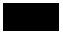 | 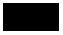 | 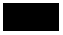 | 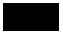 | 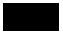 | 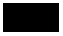 | 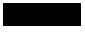 |
| 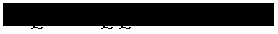 | 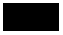 | 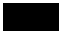 | 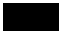 | 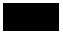 | 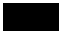 | 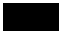 | 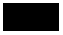 | 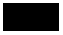 | 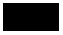 | 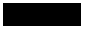 |
| 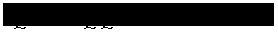 | 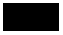 | 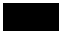 | 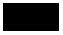 | 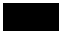 | 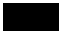 | 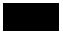 | 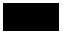 | 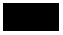 | 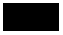 | 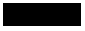 |
| 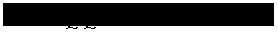 | 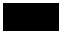 | 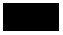 | 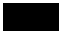 | 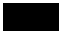 | 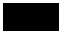 | 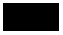 | 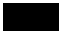 | 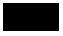 | 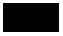 | 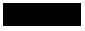 |
| EVQQELYSPLFLIVA | 0.7 | 0.6 | 0.6 | 0.5 | 0.7 | 0.6 | 0.9 | 0.3 | 0.3 | 332 |
| VQQELYSPLFLIVAA | 0.8 | 0.5 | 0.7 | 0.5 | 0.7 | 0.7 | 0.8 | 0.4 | 0.3 | 333 |
| QQELYSPLFLIVAAL | 0.7 | 0.4 | 0.6 | 0.4 | 0.6 | 0.6 | 0.7 | 0.5 | 0.3 | 1062 |
| QELYSPLFLIVAALV | 0.6 | 0.4 | 0.6 | 0.3 | 0.6 | 0.6 | 0.5 | 0.6 | 0.3 | 1063 |
| ELYSPLFLIVAALVF | 0.6 | 0.4 | 0.6 | 0.4 | 0.6 | 0.6 | 0.6 | 0.5 | 0.2 | 1064 |
| LYSPLFLIVAALVFL | 0.5 | 0.3 | 0.4 | 0.4 | 0.5 | 0.5 | 0.4 | 0.4 | 0.2 | 1065 |
| YSPLFLIVAALVFLI | 0.7 | 0.3 | 0.6 | 0.4 | 0.6 | 0.6 | 0.4 | 0.6 | 0.3 | 1066 |
| SPLFLIVAALVFLIL | 0.6 | 0.4 | 0.7 | 0.3 | 0.7 | 0.2 | 0.3 | 0.6 | 0.4 | 1067 |
| PLFLIVAALVFLILC | 0.5 | 0.1 | 0.2 | 0.5 | 0.0 | 0.1 | 0.1 | 0.2 | 0.1 | 1068 |
| LFLIVAALVFLILCF | 0.8 | 0.4 | 0.7 | 0.3 | 0.5 | 0.5 | 0.5 | 0.4 | 0.2 | 1069 |
| FLIVAALVFLILCFT | 0.7 | 0.4 | 0.7 | 0.4 | 0.7 | 0.6 | 0.5 | 0.5 | 0.3 | 1070 |
| LIVAALVFLTLCFTI | 0.6 | 0.3 | 0.5 | 0.3 | 0.6 | 0.6 | 0.5 | 0.4 | 0.2 | 1071 |
| IVAALVFLILCFTIK | 0.7 | 0.4 | 0.6 | 1.4 | 0.7 | 0.7 | 0.7 | 0.5 | 3.0 | 1072 |
| VAALVFLILCFTIKR | 0.8 | 0.4 | 0.7 | 0.6 | 0.7 | 0.7 | 0.5 | 0.4 | 0.3 | 1073 |
| AALVFLILCFTIKRK | 0.8 | 0.5 | 0.7 | 0.7 | 0.6 | 0.7 | 0.5 | 0.4 | 0.5 | 1074 |
| ALVFLILCFTIKRKT | 0.8 | 0.5 | 0.8 | 0.9 | 0.6 | 0.7 | 0.6 | 0.5 | 1.1 | 1075 |
| LVFLILCFTIKRKTE | 0.7 | 0.4 | 0.6 | 0.7 | 0.6 | 0.7 | 0.7 | 0.2 | 0.4 | 1076 |

Table 13
Binding of the sera called SARS-yellow, SARS-green, 1a,
1b, 2, 6, 37, 62 and London to linear peptides of protein X5 of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MCLKILVRYNTRGNT | 0.8 | 0.6 | 0.8 | 1.4 | 0.9 | 0.9 | 0.7 | 0.9 | 0.8 | 1077 |
| CLKILVRYNTRGNTY | 0.7 | 0.7 | 0.8 | 0.8 | 0.7 | 0.8 | 0.3 | 0.7 | 0.7 | 1078 |
| LKILVRYNTRGNTYS | 0.7 | 0.6 | 0.8 | 0.8 | 0.7 | 0.8 | 0.3 | 0.6 | 0.7 | 1079 |
| KILVRYNTRGNTYST | 0.7 | 0.5 | 0.7 | 0.7 | 0.7 | 0.8 | 0.1 | 0.5 | 0.7 | 1080 |
| ILVRYNTRGNTYSTA | 0.7 | 0.4 | 0.6 | 0.7 | 0.6 | 0.7 | 0.2 | 0.5 | 0.6 | 1081 |
| LVRYNTRGNTYSTAW | 0.7 | 0.4 | 0.5 | 0.4 | 0.6 | 0.7 | 0.3 | 0.4 | 0.6 | 1082 |
| VRYNTRGNTYSTAWL | 0.7 | 0.4 | 0.4 | 0.7 | 0.6 | 0.7 | 0.4 | 0.4 | 0.7 | 1083 |
| RYNTRGNTYSTAWLC | 0.8 | 0.4 | 0.5 | 0.9 | 0.4 | 0.7 | 0.2 | 0.6 | 1.0 | 1084 |
| YNTRGNTYSTAWLCA | 0.8 | 0.6 | 0.6 | 0.8 | 0.7 | 0.7 | 0.4 | 0.7 | 0.8 | 1085 |
| NTRGNTYSTAWLCAL | 0.9 | 0.6 | 0.6 | 0.7 | 0.7 | 0.7 | 0.5 | 0.6 | 0.7 | 1086 |
| TRGNTYSTAWLCALG | 0.8 | 0.6 | 0.6 | 0.6 | 0.8 | 0.8 | 0.5 | 0.6 | 0.7 | 1087 |
| RGNTYSTAWLCALGK | 0.9 | 0.6 | 0.9 | 0.7 | 0.8 | 0.8 | 0.3 | 0.6 | 0.8 | 1088 |
| GNTYSTAWLCALGKV | 0.8 | 0.6 | 0.9 | 0.6 | 0.7 | 0.8 | 0.4 | 0.6 | 0.7 | 1089 |
| NTYSTAWLCALGKVL | 0.7 | 0.4 | 0.5 | 0.6 | 0.6 | 0.7 | 0.3 | 0.4 | 0.6 | 1090 |
| TYSTAWLCALGKVLP | 0.8 | 0.6 | 0.9 | 0.6 | 0.7 | 0.8 | 0.4 | 0.7 | 0.7 | 1091 |
| YSTAWLCALGKVLPF | 0.7 | 0.6 | 0.7 | 0.6 | 0.6 | 0.8 | 0.4 | 0.5 | 0.8 | 1092 |
| STAWLCALGKVLPFH | 0.8 | 0.6 | 0.6 | 0.7 | 0.7 | 0.8 | 0.3 | 0.6 | 0.7 | 1093 |
| TAWLCALGKVLPFHR | 0.7 | 0.5 | 0.6 | 0.5 | 0.7 | 0.7 | 0.3 | 0.7 | 0.6 | 1094 |
| AWLCALGKVLPFHRW | 0.8 | 0.6 | 0.8 | 0.6 | 0.8 | 0.9 | 0.2 | 0.6 | 0.8 | 1095 |
| WLCALGKVLPFHRWH | 0.7 | 0.6 | 0.7 | 0.8 | 0.7 | 0.8 | 0.2 | 0.6 | 0.8 | 1096 |
| LCALGKVLPFHRWHT | 0.7 | 0.6 | 0.7 | 0.5 | 0.7 | 0.8 | 0.2 | 0.6 | 0.9 | 1097 |
| CALGKVLPFHRWHTM | 0.7 | 0.5 | 0.7 | 0.7 | 0.8 | 0.7 | 0.1 | 0.7 | 0.7 | 1098 |
| ALGKVLPFHRWHTMV | 0.9 | 0.5 | 0.7 | 0.6 | 0.6 | 0.7 | 0.2 | 0.5 | 0.7 | 1099 |
| LGKVLPFHRWHTMVQ | 0.7 | 0.4 | 0.6 | 0.5 | 0.6 | 0.6 | 0.2 | 0.4 | 0.6 | 1100 |
| GKVLPFHRWHTMVQT | 0.8 | 0.0 | 0.6 | 0.3 | 0.5 | 0.8 | 0.1 | 0.3 | 0.8 | 1101 |
| KVLPFHRWHTMVQTC | 0.9 | 0.6 | 0.7 | 0.5 | 0.7 | 0.7 | 0.2 | 0.6 | 1.2 | 1102 |
| VLPFHRWHTMVQTCT | 0.8 | 0.6 | 0.7 | 0.5 | 0.7 | 0.7 | 0.4 | 0.6 | 0.8 | 1103 |
| LPFHRWHTMVQTCTP | 0.8 | 0.7 | 0.9 | 0.6 | 0.8 | 0.8 | 0.4 | 0.6 | 0.9 | 1104 |
| PFHRWHTMVQTCTPN | 0.7 | 0.7 | 0.7 | 0.7 | 0.7 | 0.7 | 0.4 | 0.5 | 0.7 | 1105 |
| FHRWHTMVQTCTPNV | 0.8 | 0.7 | 0.9 | 0.5 | 0.7 | 0.7 | 0.4 | 0.5 | 0.7 | 1106 |
| HRWHTMVQTCTPNVT | 0.7 | 0.6 | 0.8 | 0.5 | 0.7 | 0.8 | 0.5 | 0.5 | 0.7 | 1107 |
| 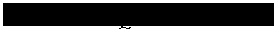 | 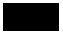 | 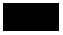 | 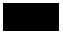 | 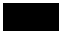 | 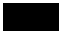 | 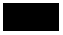 | 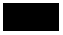 | 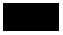 | 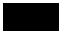 | 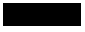 |
| 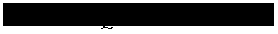 | 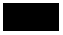 | 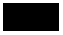 | 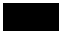 | 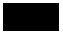 | 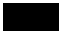 | 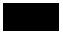 | 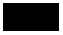 | 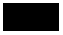 | 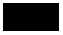 | 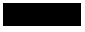 |
| 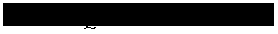 | 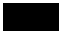 | 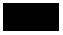 | 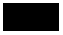 | 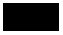 | 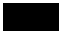 | 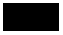 | 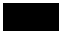 | 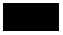 | 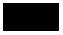 | 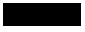 |
| 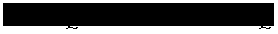 | 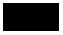 | 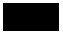 | 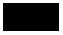 | 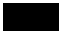 | 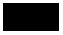 | 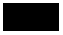 | 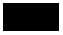 | 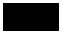 | 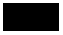 | 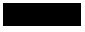 |
| 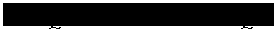 | 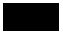 | 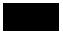 | 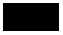 | 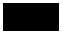 | 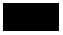 | 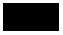 | 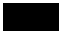 | 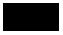 | 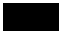 | 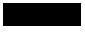 |
| VQTCTPNVTINCQDP | 0.7 | 0.8 | 0.7 | 0.8 | 0.7 | 0.8 | 0.3 | 0.7 | 0.8 | 1108 |
| QTCTPNVTINCQDPA | 0.8 | 0.7 | 0.6 | 0.8 | 0.8 | 0.8 | 0.1 | 0.8 | 0.9 | 1109 |
| TCTPNVTINCQDPAG | 0.8 | 0.6 | 0.6 | 0.8 | 0.7 | 0.8 | 0.2 | 0.8 | 0.8 | 1110 |
| CTPNVTINCQDPAGG | 1.0 | 0.8 | 0.6 | 0.6 | 0.7 | 0.7 | 0.0 | 0.5 | 0.8 | 1111 |
| TPNVTINCQDPAGGA | 0.8 | 0.4 | 0.4 | 0.1 | 0.8 | 0.5 | 0.0 | 0.4 | 0.6 | 1112 |
| 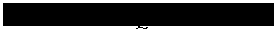 | 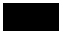 | 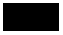 | 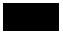 | 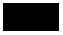 | 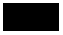 | 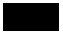 | 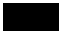 | 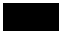 | 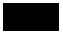 | 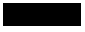 |
| 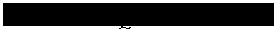 | 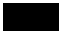 | 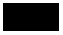 | 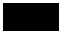 | 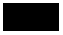 | 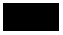 | 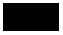 | 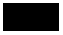 | 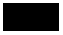 | 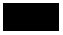 | 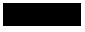 |
| 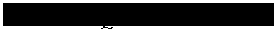 | 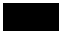 | 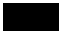 | 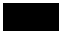 | 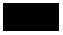 | 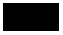 | 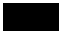 | 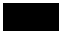 | 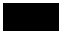 | 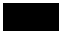 | 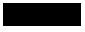 |
| 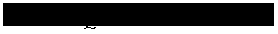 | 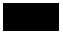 | 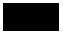 | 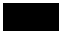 | 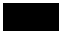 | 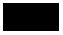 | 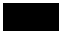 | 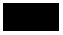 | 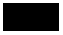 | 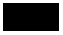 | 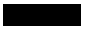 |
| 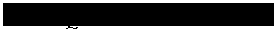 | 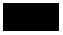 | 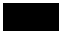 | 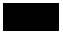 | 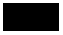 | 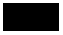 | 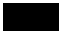 | 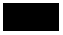 | 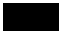 | 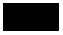 | 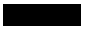 |
| 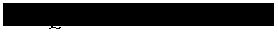 | 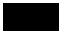 | 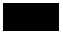 | 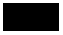 | 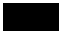 | 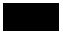 | 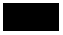 | 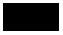 | 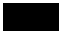 | 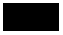 | 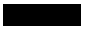 |
| 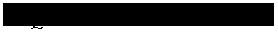 | 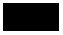 | 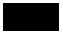 | 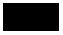 | 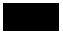 | 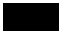 | 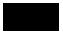 | 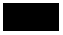 | 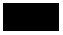 | 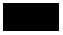 | 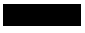 |
| 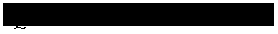 | 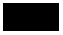 | 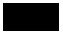 | 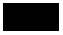 | 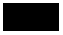 | 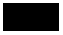 | 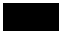 | 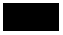 | 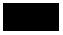 | 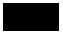 | 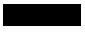 |
| DPAGGALIARCWYLH | 0.7 | 0.6 | 0.4 | 0.5 | 0.8 | 0.7 | 0.2 | 0.6 | 0.6 | 1113 |
| PAGGALIARCWYLHE | 0.8 | 0.9 | 0.6 | 0.8 | 0.9 | 0.9 | 0.3 | 0.9 | 0.7 | 1114 |
| AGGALIARCWYLHEG | 0.8 | 0.7 | 0.5 | 0.7 | 0.8 | 0.9 | 0.5 | 0.7 | 0.7 | 1115 |
| GGALIARCWYLHEGH | 0.8 | 0.7 | 0.6 | 0.8 | 0.7 | 0.8 | 0.3 | 0.7 | 0.7 | 1116 |
| GALIARCWYLHEGHQ | 0.7 | 0.6 | 0.6 | 0.7 | 0.7 | 0.7 | 0.1 | 0.6 | 0.6 | 1117 |
| ALIARCWYLHEGHQT | 0.6 | 0.5 | 0.6 | 0.5 | 0.7 | 0.7 | 0.2 | 0.7 | 0.6 | 1118 |
| LIARCWYLHEGHQTA | 0.7 | 0.6 | 0.6 | 0.1 | 0.8 | 0.9 | 0.0 | 0.6 | 0.6 | 1119 |
| 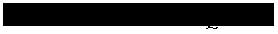 | 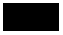 | 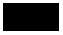 | 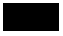 | 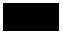 | 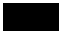 | 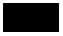 | 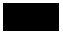 | 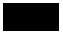 | 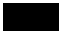 | 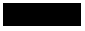 |
| 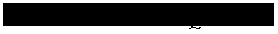 | 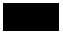 | 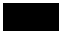 | 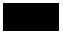 | 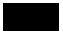 | 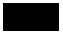 | 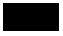 | 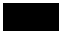 | 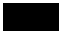 | 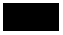 | 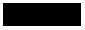 |
| 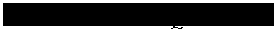 | 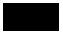 | 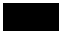 | 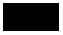 | 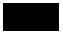 | 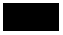 | 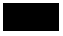 | 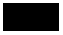 | 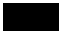 | 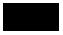 | 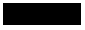 |
| 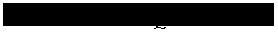 | 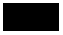 | 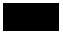 | 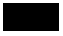 | 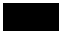 | 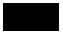 | 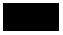 | 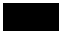 | 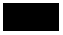 | 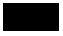 | 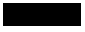 |
| 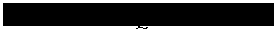 | 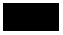 | 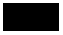 | 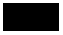 | 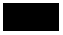 | 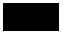 | 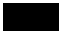 | 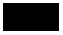 | 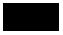 | 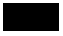 | 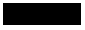 |
| 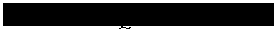 | 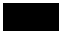 | 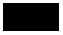 | 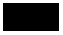 | 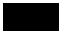 | 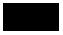 | 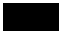 | 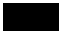 | 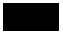 | 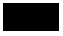 | 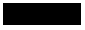 |
| 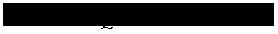 | 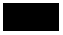 | 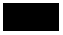 | 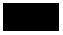 | 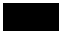 | 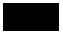 | 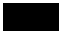 | 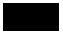 | 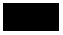 | 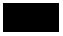 | 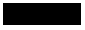 |
| 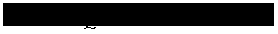 | 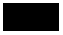 | 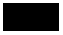 | 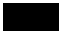 | 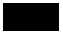 | 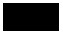 | 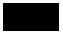 | 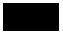 | 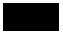 | 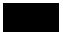 | 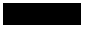 |
| 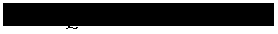 | 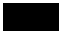 | 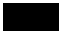 | 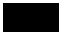 | 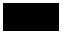 | 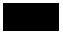 | 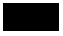 | 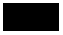 | 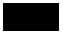 | 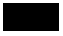 | 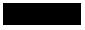 |
| 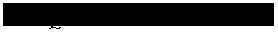 | 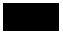 | 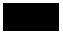 | 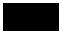 | 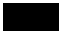 | 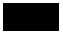 | 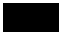 | 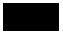 | 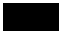 | 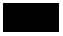 | 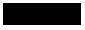 |
| 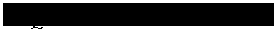 | 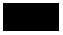 | 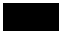 | 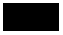 | 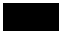 | 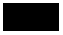 | 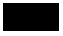 | 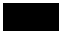 | 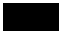 | 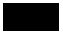 | 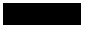 |
| QTAAFRDVLVVLNKR | 0.5 | 0.5 | 0.6 | 0.6 | 0.5 | 0.4 | 0.6 | 0.2 | 0.5 | 1120 |
| TAAFRDVLVVLNKRT | 0.5 | 0.6 | 0.6 | 0.6 | 0.5 | 0.5 | 0.7 | 0.2 | 0.5 | 1121 |
| AAFRDVLVVLNKRTN | 0.6 | 0.6 | 0.6 | 0.8 | 0.5 | 0.4 | 0.8 | 0.1 | 0.6 | 1122 |

TABLE 14
Binding of the sera called SARS-yellow, SARS-green, 1a,
1b, 2, 6, 37, 62 and London to looped/cyclic peptides of protein X5
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | yellow | green | London | NO |
| MCLKILVRYNTRGNT | 0.5 | 0.3 | 0.5 | 0.4 | 0.5 | 0.7 | 0.8 | 0.2 | 0.4 | 1077 |
| CLKILVRYNTRGNTY | 0.5 | 0.2 | 0.5 | 0.4 | 0.5 | 0.7 | 0.8 | 0.4 | 0.2 | 1078 |
| LKILVRYNTRGNTYS | 0.5 | 0.4 | 0.6 | 0.5 | 0.5 | 0.7 | 0.8 | 0.4 | 0.2 | 1079 |
| KILVRYNTRGNTYST | 0.5 | 0.3 | 0.5 | 0.5 | 0.5 | 0.7 | 0.6 | 0.3 | 0.3 | 1080 |
| ILVRYNTRGNTYSTA | 0.5 | 0.3 | 0.6 | 0.6 | 0.4 | 0.8 | 0.8 | 0.3 | 0.2 | 1081 |
| LVRYNTRGNTYSTAW | 0.4 | 0.2 | 0.4 | 0.3 | 0.5 | 0.6 | 0.5 | 0.3 | 0.2 | 1082 |
| VRYNTRGNTYSTAWL | 0.5 | 0.3 | 0.4 | 0.3 | 0.6 | 0.4 | 0.6 | 0.6 | 0.2 | 1083 |
| RYNTRGNTYSTAWLC | 0.5 | 0.0 | 0.3 | 0.1 | 0.0 | 0.2 | 0.6 | 0.1 | 0.2 | 1084 |
| YNTRGNTYSTAWLCA | 0.3 | 0.2 | 0.4 | 0.4 | 0.0 | 0.2 | 0.6 | 0.2 | 0.2 | 1085 |
| NTRGNTYSTAWLCAL | 0.4 | 0.2 | 0.4 | 0.2 | 0.5 | 0.7 | 0.5 | 0.3 | 0.1 | 1086 |
| TRGNTYSTAWLCALG | 0.4 | 0.1 | 0.4 | 0.3 | 0.4 | 0.6 | 0.3 | 0.3 | 0.2 | 1087 |
| RGNTYSTAWLCALGK | 0.4 | 0.3 | 0.6 | 0.9 | 0.5 | 0.7 | 0.5 | 0.3 | 1.4 | 1088 |
| GNTYSTAWLCALGKV | 0.4 | 0.2 | 0.5 | 0.3 | 0.5 | 0.7 | 0.5 | 0.3 | 0.2 | 1089 |
| NTYSTAWLCALGKVL | 0.4 | 0.3 | 0.5 | 1.1 | 0.5 | 0.6 | 0.6 | 0.3 | 2.3 | 1090 |
| TYSTAWLCALGKVLP | 0.5 | 0.3 | 1.1 | 0.4 | 0.6 | 0.8 | 0.5 | 0.3 | 0.2 | 1091 |
| YSTAWLCALGKVLPF | 0.5 | 0.3 | 0.5 | 0.6 | 0.5 | 0.8 | 0.9 | 0.2 | 0.4 | 1092 |
| STAWLCALGKVLPFH | 0.6 | 0.4 | 0.6 | 0.5 | 0.4 | 0.7 | 1.0 | 0.3 | 0.2 | 1093 |
| TAWLCALGKVLPFHR | 0.4 | 0.3 | 0.6 | 0.9 | 0.4 | 0.7 | 0.8 | 0.2 | 1.3 | 1094 |
| AWLCALGKVLPFHRW | 0.4 | 0.3 | 0.5 | 0.4 | 0.5 | 0.7 | 0.5 | 0.5 | 0.2 | 1095 |
| WLCALGKVLPFHRWH | 0.5 | 0.4 | 0.6 | 0.7 | 0.5 | 0.7 | 0.6 | 0.5 | 0.5 | 1096 |
| LCALGKVLPFHRWHT | 0.4 | 0.2 | 0.5 | 0.4 | 0.4 | 0.6 | 0.4 | 0.4 | 0.2 | 1097 |
| CALGKVLPFHRWHTM | 0.4 | 0.2 | 0.6 | 0.4 | 0.5 | 0.3 | 0.4 | 0.4 | 0.2 | 1098 |
| ALGKVLPFHRWHTMV | 0.4 | 0.0 | 0.6 | 0.3 | 0.5 | 0.5 | 0.1 | 0.6 | 0.2 | 1099 |
| LGKVLPFHRWHTMVQ | 0.6 | 0.3 | 0.2 | 0.3 | 0.5 | 0.3 | 0.2 | 0.2 | 0.2 | 1100 |
| GKVLPFHRWHTMVQT | 0.8 | 0.5 | 0.6 | 0.4 | 0.6 | 0.5 | 0.4 | 0.5 | 0.3 | 1101 |
| KVLPFHRWHTMVQTC | 0.7 | 0.7 | 0.6 | 0.7 | 0.6 | 0.6 | 0.3 | 0.4 | 0.4 | 1102 |
| VLPFHRWHTMVQTCT | 0.7 | 0.5 | 0.7 | 0.4 | 0.5 | 0.3 | 0.3 | 1.4 | 0.2 | 1103 |
| LPFHRWHTMVQTGTP | 0.5 | 0.4 | 0.5 | 0.3 | 0.4 | 0.3 | 0.2 | 0.3 | 0.2 | 1104 |
| PFHRWHTMVQTCTPN | 0.6 | 0.5 | 0.6 | 0.3 | 0.5 | 0.3 | 0.3 | 0.3 | 0.2 | 1105 |
| FHRWHTMVQTCTPNV | 0.7 | 0.6 | 0.6 | 0.3 | 0.5 | 0.4 | 0.4 | 0.5 | 0.2 | 1106 |
| HRWHTMVQTCTPNVT | 0.6 | 0.5 | 0.5 | 0.3 | 0.3 | 0.3 | 0.2 | 0.3 | 0.1 | 1107 |
| 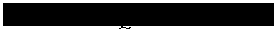 | 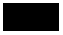 | 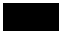 | 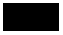 | 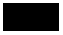 | 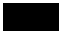 | 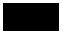 | 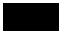 | 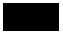 | 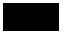 | 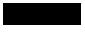 |
| 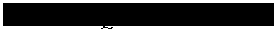 | 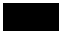 | 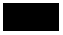 | 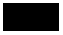 | 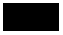 | 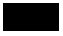 | 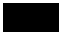 | 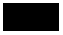 | 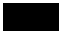 | 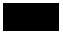 | 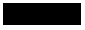 |
| 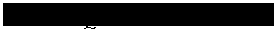 | 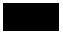 | 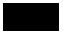 | 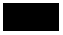 | 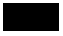 | 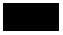 | 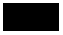 | 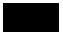 | 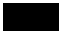 | 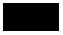 | 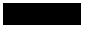 |
| 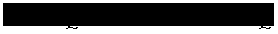 | 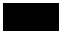 | 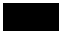 | 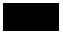 | 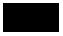 | 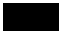 | 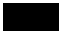 | 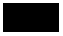 | 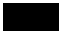 | 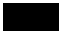 | 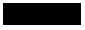 |
| 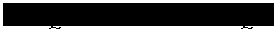 | 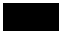 | 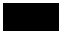 | 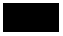 | 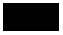 | 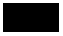 | 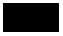 | 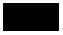 | 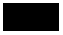 | 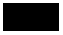 | 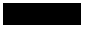 |
| VQTCTPNVTINCQDP | 0.6 | 0.5 | 0.7 | 0.2 | 0.6 | 0.6 | 0.3 | 0.3 | 0.3 | 1108 |
| QTCTPNVTINCQDPA | 0.4 | 0.1 | 0.3 | 0.2 | 0.3 | 0.2 | 0.1 | 0.0 | 0.1 | 1109 |
| TCTPNVTINCQDPAG | 0.2 | 0.0 | 0.1 | 0.1 | 0.1 | 0.1 | 0.0 | 0.2 | 0.1 | 1110 |
| CTPNVTINCQDPAGG | 0.6 | 0.8 | 0.3 | 0.3 | 0.4 | 0.3 | 0.4 | 0.4 | 0.2 | 1111 |
| TPNVTINCQDPAGGA | 0.4 | 0 4 | 0.3 | 0.1 | 0.4 | 0.2 | 0.2 | 0.2 | 0.1 | 1112 |
| 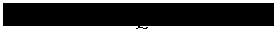 | 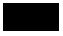 | 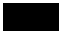 | 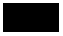 | 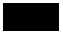 | 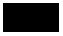 | 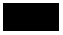 | 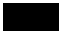 | 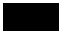 | 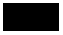 | 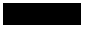 |
| 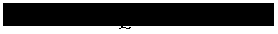 | 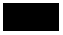 | 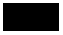 | 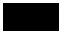 | 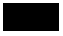 | 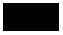 | 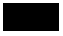 | 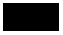 | 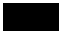 | 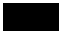 | 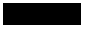 |
| 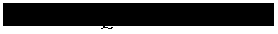 | 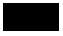 | 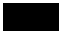 | 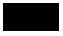 | 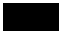 | 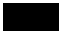 | 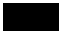 | 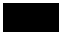 | 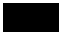 | 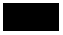 | 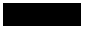 |
| 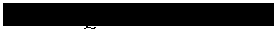 | 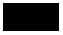 | 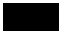 | 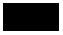 | 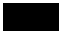 | 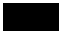 | 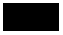 | 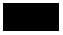 | 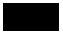 | 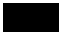 | 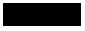 |
| 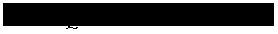 | 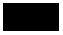 | 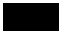 | 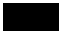 | 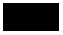 | 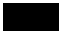 | 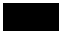 | 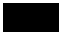 | 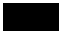 | 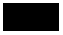 | 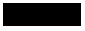 |
| 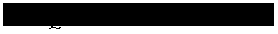 | 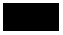 | 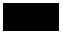 | 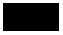 | 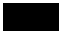 | 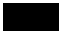 | 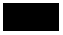 | 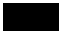 | 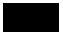 | 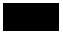 | 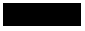 |
| 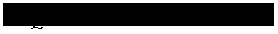 | 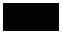 | 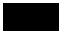 | 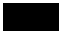 | 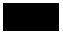 | 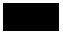 | 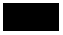 | 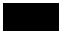 | 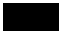 | 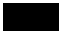 | 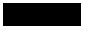 |
| 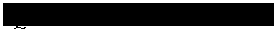 | 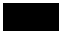 | 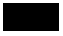 | 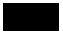 | 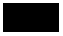 | 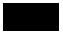 | 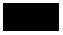 | 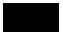 | 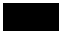 | 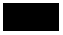 | 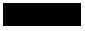 |
| DPAGGALIARCWYLH | 0.8 | 0.5 | 0.5 | 0.4 | 0.7 | 0.6 | 0.3 | 0.6 | 0.2 | 1113 |
| PAGGALIARCWYLHE | 0.8 | 0.6 | 0.6 | 0.4 | 0.7 | 0.7 | 0.5 | 0.6 | 0.3 | 1114 |
| AGGALIARCWYLHEG | 0.7 | 0.4 | 0.5 | 0.5 | 0.6 | 0.5 | 0.4 | 0.4 | 0.2 | 1115 |
| GGALIARCWYLHEGH | 0.7 | 0.6 | 0.6 | 0.4 | 0.7 | 0.7 | 0.3 | 0.7 | 0.3 | 1116 |
| GALIARCWYLHEGHQ | 0.4 | 0.4 | 0.7 | 0.3 | 0.4 | 0.3 | 0.3 | 0.8 | 0.1 | 1117 |
| ALIARCWYLHEGHQT | 0.6 | 0.1 | 0.5 | 0.3 | 0.4 | 0.4 | 0.0 | 0.0 | 0.1 | 1118 |
| LIARCWYLHEGHQTA | 0.4 | 0.4 | 0.2 | 0.1 | 0.3 | 0.1 | 0.0 | 0.6 | 0.0 | 1119 |
| 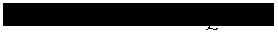 | 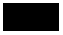 | 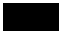 | 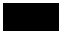 | 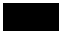 | 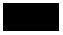 | 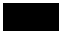 | 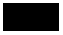 | 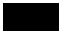 | 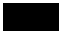 | 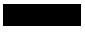 |
| 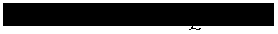 | 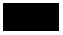 | 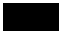 | 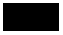 | 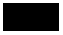 | 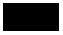 | 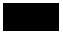 | 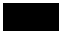 | 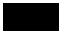 | 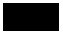 | 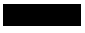 |
| 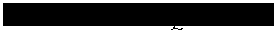 | 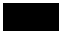 | 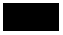 | 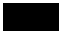 | 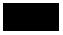 | 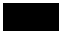 | 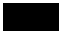 | 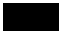 | 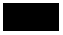 | 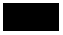 | 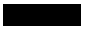 |
| 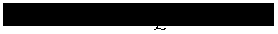 | 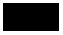 | 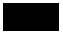 | 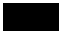 | 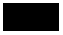 | 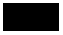 | 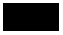 | 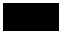 | 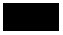 | 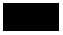 | 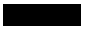 |
| 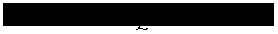 | 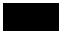 | 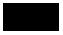 | 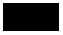 | 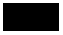 | 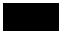 | 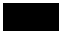 | 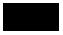 | 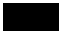 | 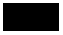 | 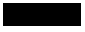 |
| 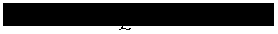 | 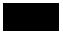 | 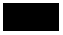 | 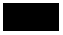 | 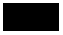 | 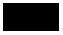 | 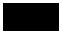 | 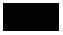 | 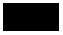 | 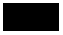 | 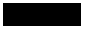 |
| 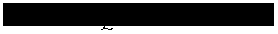 | 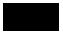 | 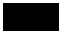 | 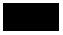 | 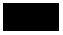 | 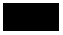 | 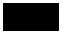 | 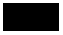 | 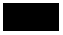 | 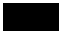 | 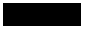 |
| 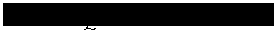 | 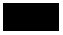 | 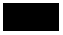 | 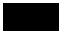 | 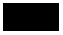 | 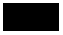 | 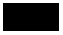 | 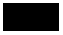 | 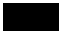 | 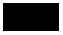 | 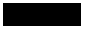 |
| 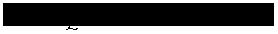 | 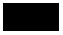 | 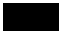 | 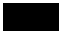 | 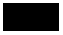 | 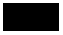 | 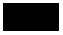 | 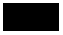 | 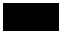 | 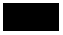 | 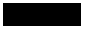 |
| 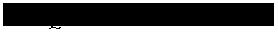 | 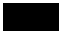 | 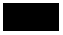 | 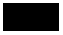 | 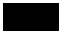 | 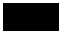 | 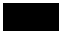 | 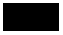 | 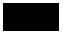 | 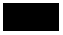 | 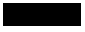 |
| 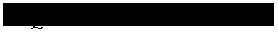 | 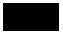 | 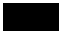 | 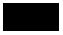 | 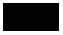 | 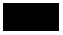 | 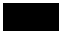 | 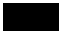 | 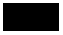 | 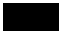 | 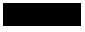 |
| QTAAFRDVLVVLNKR | 0.6 | 0.5 | 0.6 | 0.8 | 0.6 | 0.6 | 0.4 | 0.3 | 0.5 | 1120 |
| TAAFRDVLVVLNKRT | 0.7 | 0.6 | 0.7 | 0.9 | 0.6 | 0.6 | 0.5 | 0.5 | 1.1 | 1121 |
| AAFRDVLVVLNKRTN | 0.6 | 0.4 | 0.6 | 0.8 | 0.6 | 0.5 | 0.3 | 0.4 | 1.0 | 1122 |

TABLE 15
Binding of the sera called SARS-yellow, SARS-green, 1a,
1b, 2, 6, 37, 62 and London to linear peptides of protein N of
SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| MSDNGPQSNQRSAPR | 0.2 | 0.1 | 0.2 | 0.2 | 0.6 | 0.6 | 0.5 | 0.5 | 0.3 | 1123 |
| SDNGPQSNQRSAPRI | 0.2 | 0.0 | 0.1 | 0.2 | 0.2 | 0.4 | 0.5 | 0.5 | 0.0 | 1124 |
| DNGPQSNQRSAPRIT | 0.2 | 0.1 | 0.3 | 0.2 | 0.6 | 0.5 | 0.4 | 0.5 | 0.4 | 1125 |
| NGPQSNQRSAPRITF | 0.2 | 0.1 | 0.2 | 0.2 | 0.8 | 0.5 | 0.4 | 0.5 | 0.4 | 592 |
| GPQSNQRSAPRITFG | 0.2 | 0.1 | 0.2 | 0.1 | 0.6 | 0.7 | 0.4 | 0.5 | 0.4 | 593 |
| PQSNQRSAPRITFGG | 0.3 | 0.2 | 0.2 | 0.1 | 0.6 | 0.6 | 0.4 | 0.5 | 0.3 | 594 |
| QSNQRSAPRITFGGP | 0.2 | 0.1 | 0.2 | 0.1 | 0.7 | 0.7 | 0.4 | 0.5 | 0.5 | 595 |
| SNQRSAPRITFGGPT | 0.2 | 0.1 | 0.2 | 0.1 | 0.6 | 0.6 | 0.4 | 0.5 | 0.2 | 596 |
| NQRSAPRITFGGPTD | 0.2 | 0.1 | 0.1 | 0.1 | 0.7 | 0.6 | 0.5 | 0.5 | 0.3 | 597 |
| QRSAPRITFGGPTDS | 0.2 | 0.1 | 0.1 | 0.1 | 0.6 | 0.6 | 0.5 | 0.5 | 0.2 | 598 |
| RSAPRITFGGPTDST | 0.2 | 0.1 | 0.1 | 0.1 | 0.6 | 0.6 | 0.5 | 0.5 | 0.4 | 599 |
| SAPRITFGGPTDSTD | 0.2 | 0.2 | 0.1 | 0.1 | 0.7 | 0.6 | 0.5 | 0.4 | 0.6 | 600 |
| APRITFGGPTDSTDN | 0.2 | 0.2 | 0.2 | 0.2 | 0.8 | 0.7 | 0.6 | 0.3 | 0.6 | 601 |
| PRITFGGPTDSTDNN | 0.2 | 0.1 | 0.2 | 0.1 | 0.7 | 0.6 | 0.5 | 0.3 | 0.6 | 602 |
| RITFGGPTDSTDNNQ | 0.2 | 0.1 | 0.2 | 0.1 | 0.8 | 0.6 | 0.5 | 1.3 | 0.5 | 603 |
| ITFGGPTDSTDNNQN | 0.2 | 0.1 | 0.1 | 0.1 | 0.7 | 0.6 | 0.5 | 0.4 | 0.3 | 604 |
| TFGGPTDSTDNNQNG | 0.3 | 0.1 | 0.2 | 0.2 | 0.8 | 0.6 | 0.6 | 0.5 | 0.5 | 1126 |
| FGGPTDSTDNNQNGG | 0.3 | 0.1 | 0.1 | 0.1 | 0.6 | 0.5 | 0.5 | 0.5 | 0.0 | 1127 |
| GGPTDSTDNNQNGGR | 0.3 | 0.1 | 0.3 | 0.2 | 0.7 | 0.7 | 0.6 | 0.6 | 0.6 | 1128 |
| GPTDSTDNNQNGGRN | 0.3 | 0.2 | 0.4 | 0.2 | 0.8 | 0.6 | 0.6 | 0.6 | 0.6 | 1129 |
| PTDSTDNNQNGGRNG | 0.3 | 0.2 | 0.3 | 0.2 | 1.2 | 0.8 | 0.7 | 0.7 | 0.6 | 1130 |
| TDSTDNNQNGGRNGA | 0.3 | 0.2 | 0.2 | 0.3 | 1.0 | 0.8 | 0.6 | 0.7 | 1.0 | 1131 |
| DSTDNNQNGGRNGAR | 0.2 | 0.1 | 0.2 | 0.2 | 1.0 | 0.8 | 0.5 | 0.5 | 0.5 | 1132 |
| STDNNQNGGRNGARP | 0.2 | 0.1 | 0.2 | 0.1 | 0.8 | 0.7 | 0.5 | 0.6 | 0.4 | 1133 |
| TDNNQNGGRNGARPK | 0.3 | 0.2 | 0.4 | 0.2 | 0.8 | 1.0 | 0.6 | 0.7 | 0.8 | 1134 |
| DNNQNGGRNGARPKQ | 0.2 | 0.1 | 0.3 | 0.1 | 0.6 | 0.7 | 0.5 | 0.6 | 0.6 | 1135 |
| NNQNGGRNGARPKQR | 0.2 | 0.2 | 0.2 | 0.3 | 0.7 | 0.8 | 0.5 | 0.6 | 0.5 | 1136 |
| NQNGGRNGARPKQRR | 0.2 | 0.1 | 0.3 | 0.3 | 0.8 | 0.7 | 0.5 | 0.6 | 0.5 | 1137 |
| QNGGRNGARPKQRRP | 0.2 | 0.1 | 0.3 | 0.2 | 0.7 | 0.8 | 0.5 | 0.7 | 0.5 | 1138 |
| NGGRNGARPKQRRPQ | 0.2 | 0.1 | 0.2 | 0.2 | 0.7 | 0.7 | 0.5 | 0.5 | 0.5 | 1139 |
| GGRNGARPKQRRPQG | 0.3 | 0.2 | 0.3 | 0.2 | 0.7 | 0.8 | 0.5 | 0.6 | 2.6 | 1140 |
| GRNGARPKQRRPQGL | 0.2 | 0.1 | 0.2 | 0.2 | 0.7 | 0.6 | 0.4 | 0.4 | 0.4 | 1141 |
| RNGARPKQRRPQGLP | 0.2 | 0.1 | 0.2 | 0.2 | 0.6 | 0.6 | 0.5 | 0.5 | 0.4 | 1142 |
| NGARPKQRRPQGLPN | 0.2 | 0.1 | 0.2 | 0.1 | 0.7 | 0.5 | 0.4 | 0.5 | 0.3 | 1143 |
| GARPKQRRPQGLPNN | 0.2 | 0.1 | 0.2 | 0.1 | 0.4 | 0.6 | 0.5 | 0.6 | 0.3 | 1144 |
| ARPKQRRPQGLPNNT | 0.2 | 0.1 | 0.3 | 0.2 | 0.7 | 0.6 | 0.5 | 0.5 | 0.5 | 1145 |
| RPKQRRPQGLPNNTA | 0.3 | 0.1 | 0.3 | 0.2 | 0.7 | 0.6 | 0.5 | 0.7 | 0.5 | 1146 |
| PKQRRPQGLPNNTAS | 0.2 | 0.1 | 0.4 | 0.2 | 0.9 | 0.7 | 0.6 | 0.6 | 0.6 | 1147 |
| KQRRPQGLPNNTASW | 0.2 | 0.1 | 0.2 | 0.1 | 0.7 | 0.6 | 0.4 | 0.5 | 0.3 | 1148 |
| QRRPQGLPNNTASWF | 0.2 | 0.1 | 0.1 | 0.2 | 0.6 | 0.7 | 0.4 | 0.7 | 0.2 | 1149 |
| RRPQGLPNNTASWFT | 0.2 | 0.1 | 0.1 | 0.1 | 0.6 | 0.6 | 0.4 | 0.6 | 0.2 | 1150 |
| RPQGLPNNTASWFTA | 0.3 | 0.1 | 0.2 | 0.2 | 0.7 | 0.7 | 0.5 | 0.4 | 0.3 | 1151 |
| PQGLPNNTASWFTAL | 0.2 | 0.1 | 0.2 | 0.1 | 0.7 | 0.7 | 0.4 | 0.5 | 0.2 | 1152 |
| QGLPNNTASWFTALT | 0.2 | 0.1 | 0.2 | 0.1 | 0.6 | 0.7 | 0.4 | 0.6 | 0.2 | 1153 |
| GLPNNTASWFTALTQ | 0.2 | 0.1 | 0.2 | 0.1 | 0.7 | 0.6 | 0.4 | 0.4 | 0.4 | 1154 |
| LPNNTASWFTALTQH | 0.2 | 0.1 | 0.2 | 0.1 | 0.7 | 0.7 | 0.4 | 0.5 | 0.3 | 1155 |
| PNNTASWFTALTQHG | 0.2 | 0.1 | 0.2 | 0.2 | 0.7 | 0.7 | 0.5 | 0.4 | 0.3 | 1156 |
| NNTASWFTALTQHGK | 0.2 | 0.1 | 0.2 | 0.1 | 0.7 | 0.6 | 0.4 | 0.5 | 0.4 | 1157 |
| NTASWFTALTQHGKE | 0.2 | 0.1 | 0.1 | 0.1 | 0.7 | 0.5 | 0.4 | 0.5 | 0.4 | 1158 |
| TASWFTALTQHGKEE | 0.2 | 0.1 | 0.1 | 0.1 | 0.7 | 0.4 | 0.4 | 0.5 | 0.3 | 1159 |
| ASWFTALTQHGKEEL | 0.2 | 0.1 | 0.1 | 0.1 | 0.9 | 0.6 | 0.5 | 0.4 | 0.4 | 1160 |
| SWFTALTQHGKEELR | 0.2 | 0.1 | 0.2 | 0.1 | 0.6 | 0.6 | 0.5 | 0.5 | 0.2 | 1161 |
| WFTALTQHGKEELRF | 0.3 | 0.2 | 0.3 | 0.2 | 1.2 | 0.9 | 0.7 | 0.5 | 0.4 | 1162 |
| FTALTQHGKEELRFP | 0.3 | 0.2 | 0.3 | 0.2 | 0.8 | 0.7 | 0.5 | 0.6 | 0.7 | 1163 |
| TALTQHGKEELRFPR | 0.2 | 0.1 | 0.2 | 0.1 | 0.7 | 0.6 | 0.5 | 0.5 | 0.4 | 1164 |
| ALTQHGKEELRFPRG | 0.3 | 0.2 | 0.3 | 0.2 | 0.9 | 0.8 | 0.5 | 0.6 | 0.6 | 1165 |
| LTQHGKEELRFPRGQ | 0.2 | 0.1 | 0.3 | 0.2 | 0.6 | 0.7 | 0.4 | 0.6 | 0.3 | 1166 |
| TQHGKEELRFPRGQG | 0.3 | 0.2 | 0.3 | 0.2 | 0.7 | 0.8 | 0.5 | 0.6 | 0.4 | 1167 |
| QHGKEELRFPRGQGV | 0.2 | 0.2 | 0.3 | 0.1 | 0.7 | 0.7 | 0.5 | 0.6 | 0.4 | 1168 |
| HGKEELRFPRGQGVP | 0.2 | 0.2 | 0.3 | 0.1 | 0.7 | 0.7 | 0.5 | 0.7 | 0.5 | 1169 |
| GKEELRFPRGQGVPI | 0.3 | 0.1 | 0.3 | 0.2 | 0.7 | 0.7 | 0.5 | 0.6 | 0.6 | 1170 |
| KEELRFPRGQGVPIN | 0.2 | 0.1 | 0.3 | 0.2 | 0.7 | 0.6 | 0.5 | 0.5 | 0.5 | 1171 |
| EELRFPRGQGVPINT | 0.2 | 0.1 | 0.3 | 0.2 | 0.7 | 0.7 | 0.5 | 0.6 | 0.4 | 1172 |
| ELRFPRGQGVPINTN | 0.2 | 0.1 | 0.3 | 0.1 | 0.7 | 0.7 | 0.5 | 0.6 | 0.4 | 1173 |
| LRFPRGQGVPINTNS | 0.2 | 0.1 | 0.3 | 0.2 | 0.6 | 0.6 | 0.5 | 0.5 | 0.4 | 1174 |
| RFPRGQGVPINTNSG | 0.2 | 0.1 | 0.3 | 0.1 | 0.7 | 0.7 | 0.5 | 0.6 | 0.4 | 1175 |
| FPRGQGVPINTNSGP | 0.2 | 0.1 | 0.3 | 0.2 | 0.7 | 0.6 | 0.5 | 0.6 | 0.3 | 1176 |
| PRGQGVPINTNSGPD | 0.2 | 0.1 | 0.1 | 0.1 | 0.6 | 0.4 | 0.4 | 0.6 | 0.2 | 1177 |
| RGQGVPINTNSGPDD | 0.2 | 0.1 | 0.1 | 0.1 | 0.3 | 0.4 | 0.4 | 0.6 | 0.4 | 1178 |
| GQGVPINTNSGPDDQ | 0.3 | 0.1 | 0.2 | 0.2 | 0.8 | 0.6 | 0.6 | 0.6 | 0.5 | 1179 |
| QGVPINTNSGPDDQI | 0.3 | 0.2 | 0.2 | 0.2 | 0.9 | 0.6 | 0.5 | 0.6 | 0.6 | 1180 |
| GVPINTNSGPDDQIG | 0.3 | 0.1 | 0.2 | 0.2 | 0.8 | 0.6 | 0.5 | 0.5 | 0.5 | 1181 |
| VPINTNSGPDDQIGY | 0.3 | 0.2 | 0.2 | 0.2 | 0.8 | 0.7 | 0.4 | 0.5 | 0.4 | 1182 |
| PINTNSGPDDQIGYY | 0.2 | 0.2 | 0.2 | 0.2 | 0.9 | 0.7 | 0.4 | 0.6 | 0.3 | 1183 |
| INTNSGPDDQIGYYR | 0.3 | 0.2 | 0.2 | 0.2 | 0.9 | 0.7 | 0.4 | 0.7 | 0.2 | 1184 |
| NTNSGPDDQIGYYRR | 0.2 | 0.1 | 0.2 | 0.2 | 0.6 | 0.6 | 0.4 | 0.7 | 0.3 | 1185 |
| TNSGPDDQIGYYRRA | 0.3 | 0.2 | 0.2 | 0.2 | 0.7 | 0.7 | 0.5 | 0.6 | 0.4 | 1186 |
| NSGPDDQIGYYRRAT | 0.2 | 0.1 | 0.1 | 0.2 | 0.7 | 0.6 | 0.4 | 0.5 | 0.1 | 1187 |
| SGPDDQTGYYRRATR | 0.2 | 0.1 | 0.2 | 0.2 | 0.6 | 0.6 | 0.4 | 0.6 | 0.4 | 545 |
| GPDDQIGYYRRATRR | 0.2 | 0.1 | 0.2 | 0.2 | 0.6 | 0.6 | 0.4 | 0.6 | 0.3 | 546 |
| PDDQIGYYRRATRRV | 0.2 | 0.1 | 0.2 | 0.2 | 0.6 | 0.5 | 0.4 | 0.6 | 0.3 | 547 |
| DDQIGYYRRATRRVR | 0.3 | 0.1 | 0.3 | 0.3 | 0.7 | 0.9 | 0.7 | 0.6 | 0.6 | 548 |
| DQIGYYRRATRRVRG | 0.2 | 0.1 | 0.3 | 0.2 | 0.7 | 0.6 | 0.5 | 0.6 | 0.4 | 549 |
| QIGYYRRATRRVRGG | 0.3 | 0.1 | 0.2 | 0.2 | 0.6 | 0.6 | 0.5 | 0.6 | 0.2 | 550 |
| IGYYRRATRRVRGGD | 0.2 | 0.1 | 0.1 | 0.2 | 0.5 | 0.4 | 0.4 | 0.6 | 0.2 | 551 |
| GYYRRATRRVRGGDG | 0.3 | 0.1 | 0.1 | 0.2 | 0.3 | 0.4 | 0.4 | 0.6 | 0.2 | 552 |
| YYRRATRRVRGGDGK | 0.3 | 0.1 | 0.3 | 0.3 | 0.7 | 0.6 | 0.5 | 0.5 | 0.3 | 1188 |
| YRRATRRVRGGDGKM | 0.3 | 0.1 | 0.3 | 0.3 | 0.8 | 0.7 | 0.5 | 0.6 | 0.4 | 1189 |
| RRATRRVRGGDGKMK | 0.2 | 0.1 | 0.3 | 0.3 | 0.8 | 0.7 | 0.5 | 0.6 | 0.3 | 1190 |
| RATRRVRGGDGKMKE | 0.2 | 0.1 | 0.4 | 0.2 | 0.8 | 0.7 | 0.5 | 0.6 | 0.3 | 1191 |
| ATRRVRGGDGKMKEL | 0.3 | 0.1 | 0.3 | 0.3 | 0.8 | 0.7 | 0.5 | 0.6 | 0.3 | 1192 |
| TRRVRGGDGKMKELS | 0.3 | 0.2 | 0.3 | 0.3 | 0.7 | 0.8 | 0.5 | 0.7 | 0.3 | 1193 |
| RRVRGGDGKMKELSP | 0.3 | 0.2 | 0.4 | 0.2 | 0.7 | 0.8 | 0.6 | 0.8 | 0.4 | 1194 |
| RVRGGDGKMKELSPR | 0.3 | 0.2 | 0.3 | 0.2 | 0.7 | 0.8 | 0.5 | 0.7 | 0.6 | 1195 |
| VRGGDGKMKELSPRW | 0.2 | 0.1 | 0.2 | 0.2 | 0.6 | 0.6 | 0.4 | 0.7 | 0.4 | 1196 |
| RGGDGKMKELSPRWY | 0.2 | 0.1 | 0.2 | 0.2 | 0.7 | 0.6 | 0.5 | 0.6 | 0.3 | 1197 |
| GGDGKMKELSPRWYF | 0.3 | 0.1 | 0.2 | 0.2 | 0.6 | 0.7 | 0.4 | 0.7 | 0.2 | 1198 |
| GDGKMKELSPRWYFY | 0.2 | 0.1 | 0.2 | 0.1 | 0.5 | 0.6 | 0.4 | 0.6 | 0.3 | 1199 |
| DGKMKELSPRWYFYY | 0.2 | 0.1 | 0.2 | 0.1 | 0.5 | 0.5 | 0.4 | 0.5 | 0.4 | 1200 |
| GKMKELSPRWYFYYL | 0.2 | 0.1 | 0.1 | 0.1 | 0.5 | 0.5 | 0.4 | 0.6 | 0.2 | 1201 |
| KMKELSPRWYFYYLG | 0.2 | 0.1 | 0.1 | 0.1 | 0.5 | 0.5 | 0.4 | 0.6 | 0.1 | 1202 |
| MKELSPRWYFYYLGT | 0.2 | 0.1 | 0.1 | 0.1 | 0.5 | 0.4 | 0.4 | 0.6 | 0.2 | 1203 |
| KELSPRWYFYYLGTG | 0.2 | 0.1 | 0.1 | 0.1 | 0.4 | 0.4 | 0.4 | 0.5 | 0.1 | 1204 |
| ELSPRWYFYYLGTGP | 0.2 | 0.2 | 0.3 | 0.2 | 0.8 | 0.7 | 0.5 | 0.7 | 0.4 | 1205 |
| LSPRWYFYYLGTGPE | 0.3 | 0.3 | 0.2 | 0.2 | 1.0 | 0.9 | 0.5 | 0.6 | 0.9 | 1206 |
| SPRWYFYYLGTGPEA | 0.3 | 0.2 | 0.2 | 0.2 | 0.9 | 0.9 | 0.5 | 0.6 | 0.6 | 1207 |
| PRWYFYYLGTGPEAS | 0.3 | 0.2 | 0.2 | 0.2 | 0.9 | 0.7 | 0.5 | 0.6 | 0.5 | 1208 |
| RWYFYYLGTGPEASL | 0.3 | 0.2 | 0.2 | 0.2 | 0.8 | 0.8 | 0.4 | 0.6 | 0.3 | 1209 |
| WYFYYLGTGPEASLP | 0.3 | 0.2 | 0.3 | 0.2 | 0.9 | 0.8 | 0.5 | 0.7 | 0.3 | 1210 |
| YFYYLGTGPEASLPY | 0.3 | 0.1 | 0.3 | 0.2 | 0.7 | 0.8 | 0.4 | 0.7 | 0.0 | 1211 |
| FYYLGTGPEASLPYG | 0.3 | 0.1 | 0.2 | 0.2 | 0.6 | 0.7 | 0.5 | 0.7 | 0.4 | 1212 |
| YYLGTGPEASLPYGA | 0.3 | 0.2 | 0.2 | 0.2 | 0.7 | 0.7 | 0.4 | 0.8 | 0.2 | 1213 |
| YLGTGPEASLPYGAN | 0.2 | 0.1 | 0.2 | 0.2 | 0.7 | 0.6 | 0.5 | 0.6 | 0.3 | 1214 |
| LGTGPEASLPYGANK | 0.3 | 0.1 | 0.3 | 0.2 | 0.7 | 0.7 | 0.5 | 0.7 | 0.5 | 1215 |
| GTGPEASLPYGANKE | 0.3 | 0.1 | 0.3 | 0.2 | 0.9 | 0.9 | 0.7 | 0.6 | 0.7 | 1216 |
| TGPEASLPYGANKEG | 0.3 | 0.2 | 0.3 | 0.3 | 0.7 | 0.9 | 0.7 | 0.6 | 0.8 | 1217 |
| GPEASLPYGANKEGI | 0.3 | 0.2 | 0.3 | 0.3 | 0.8 | 0.9 | 0.6 | 0.6 | 0.7 | 1218 |
| PEASLPYGANKEGIV | 0.3 | 0.1 | 0.2 | 0.2 | 0.7 | 0.7 | 0.5 | 0.6 | 0.3 | 1219 |
| EASLPYGANKEGIVW | 0.2 | 0.1 | 0.2 | 0.1 | 0.5 | 0.5 | 0.4 | 0.6 | 0.2 | 1220 |
| ASLPYGANKEGIVWV | 0.2 | 0.1 | 0.2 | 0.1 | 0.6 | 0.5 | 0.4 | 0.5 | 0.1 | 1221 |
| SLPYGANKEGIVWVA | 0.3 | 0.1 | 0.2 | 0.2 | 0.8 | 1.2 | 0.6 | 0.7 | 0.5 | 1222 |
| LPYGANKEGIVWVAT | 0.2 | 0.2 | 0.3 | 0.2 | 0.7 | 0.7 | 0.5 | 0.6 | 0.3 | 1223 |
| PYGANKEGIVWVATE | 0.3 | 0.2 | 0.2 | 0.2 | 1.0 | 0.8 | 0.7 | 0.6 | 0.8 | 1224 |
| YGANKEGIVWVATEG | 0.3 | 0.1 | 0.2 | 0.2 | 0.8 | 0.7 | 0.4 | 0.7 | 0.4 | 1225 |
| GANKEGIVWVATEGA | 0.2 | 0.1 | 0.2 | 0.1 | 0.7 | 0.6 | 0.4 | 0.6 | 0.2 | 1226 |
| ANKEGIVWVATEGAL | 0.2 | 0.1 | 0.2 | 0.2 | 0.8 | 0.7 | 0.4 | 0.7 | 0.4 | 1227 |
| NKEGIVWVATEGALN | 0.3 | 0.1 | 0.2 | 0.2 | 0.7 | 0.6 | 0.4 | 0.9 | 0.4 | 1228 |
| KEGIVWVATEGALNT | 0.2 | 0.1 | 0.2 | 0.2 | 0.7 | 0.6 | 0.4 | 0.7 | 0.5 | 1229 |
| EGIVWVATEGALNTP | 0.3 | 0.2 | 0.3 | 0.2 | 0.8 | 0.7 | 0.5 | 0.6 | 0.4 | 1230 |
| GIVWVATEGALNTPK | 0.3 | 0.2 | 0.5 | 0.3 | 0.9 | 0.9 | 0.6 | 0.8 | 1.0 | 1231 |
| IVWVATEGALNTPKD | 0.2 | 0.1 | 0.2 | 0.2 | 0.7 | 0.7 | 0.6 | 0.6 | 0.3 | 1232 |
| VWVATEGALNTPKDH | 0.2 | 0.1 | 0.2 | 0.2 | 0.8 | 0.7 | 0.6 | 0.6 | 0.5 | 1233 |
| WVATEGALNTPKDHI | 0.2 | 0.1 | 0.3 | 0.2 | 0.7 | 0.9 | 0.6 | 0.6 | 0.6 | 1234 |
| VATEGALNTPKDHIG | 0.3 | 0.1 | 0.2 | 0.2 | 0.7 | 1.1 | 0.7 | 0.6 | 0.5 | 1235 |
| ATEGALNTPKDHIGT | 0.2 | 0.1 | 0.3 | 0.1 | 0.7 | 0.7 | 0.5 | 0.6 | 0.4 | 1236 |
| TEGALNTPKDHIGTR | 0.2 | 0.1 | 0.2 | 0.2 | 0.7 | 0.7 | 0.5 | 0.6 | 0.6 | 1237 |
| EGALNTPKDHIGTRN | 0.2 | 0.1 | 0.2 | 0.2 | 0.6 | 0.6 | 0.6 | 0.5 | 0.1 | 1238 |
| GALNTPKDHIGTRNP | 0.3 | 0.1 | 0.2 | 0.2 | 0.7 | 0.7 | 0.5 | 0.7 | 0.4 | 1239 |
| ALNTPKDHIGTRNPN | 0.3 | 0.1 | 0.3 | 0.2 | 0.7 | 0.6 | 0.5 | 0.5 | 0.4 | 1240 |
| LNTPKDHTGTRNPNN | 0.2 | 0.1 | 0.3 | 0.2 | 0.7 | 0.6 | 0.5 | 0.6 | 0.2 | 1241 |
| NTPKDHIGTRNPNNN | 0.3 | 0.1 | 0.3 | 0.2 | 0.8 | 0.7 | 0.4 | 0.6 | 0.4 | 1242 |
| TPKDHIGTRNPNNNA | 0.3 | 0.1 | 0.3 | 0.2 | 0.9 | 0.8 | 0.5 | 0.7 | 0.4 | 1243 |
| PKDHIGTRNPNNNAA | 0.3 | 0.1 | 0.3 | 0.2 | 0.8 | 0.9 | 0.5 | 0.8 | 0.4 | 1244 |
| KDHIGTRNPNNNAAT | 0.3 | 0.2 | 0.4 | 0.3 | 0.9 | 0.8 | 0.5 | 0.8 | 0.5 | 1245 |
| DHIGTRNPNNNAATV | 0.3 | 0.1 | 0.3 | 0.2 | 0.7 | 0.7 | 0.5 | 0.7 | 0.5 | 1246 |
| HIGTRNPNNNAATVL | 0.3 | 0.2 | 0.4 | 0.2 | 0.9 | 0.8 | 0.5 | 0.7 | 0.5 | 1247 |
| IGTRNPNNNAATVLQ | 0.3 | 0.1 | 0.4 | 0.2 | 0.7 | 0.8 | 0.5 | 0.6 | 0.5 | 1248 |
| GTRNPNNNAATVLQL | 0.3 | 0.1 | 0.3 | 0.2 | 0.7 | 0.7 | 0.5 | 0.6 | 0.3 | 1249 |
| TRNPNNNAATVLQLP | 0.3 | 0.1 | 0.3 | 0.2 | 0.6 | 0.7 | 0.5 | 0.7 | 0.3 | 1250 |
| RNPNNNAATVLQLPQ | 0.2 | 0.1 | 0.3 | 0.2 | 0.6 | 0.7 | 0.5 | 0.6 | 0.3 | 1251 |
| NPNNNAATVLQLPQG | 0.4 | 0.2 | 0.4 | 0.3 | 0.8 | 0.9 | 0.8 | 0.8 | 0.6 | 1252 |
| PNNNAATVLQLPQGT | 0.2 | 0.1 | 0.2 | 0.1 | 0.6 | 0.6 | 0.5 | 0.7 | 0.3 | 1253 |
| NNNAATVLQLPQGTT | 0.2 | 0.1 | 0.2 | 0.1 | 0.7 | 0.5 | 0.5 | 0.7 | 0.4 | 1254 |
| 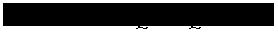 | 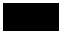 | 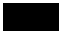 | 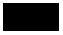 | 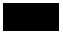 | 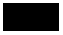 | 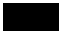 | 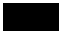 | 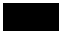 | 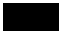 | 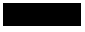 |
| 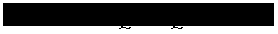 | 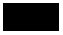 | 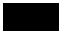 | 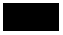 | 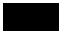 | 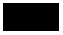 | 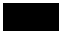 | 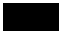 | 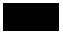 | 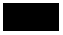 | 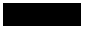 |
| 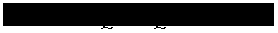 | 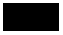 | 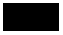 | 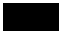 | 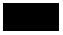 | 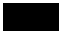 | 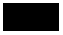 | 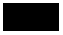 | 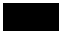 | 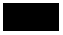 | 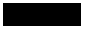 |
| 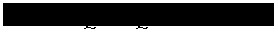 | 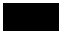 | 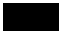 | 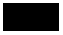 | 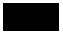 | 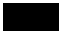 | 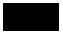 | 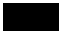 | 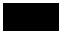 | 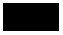 | 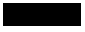 |
| 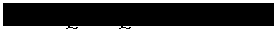 | 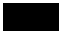 | 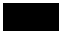 | 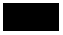 | 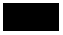 | 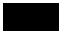 | 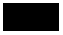 | 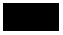 | 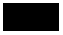 | 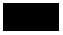 | 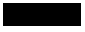 |
| 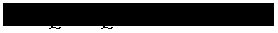 | 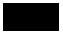 | 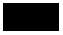 | 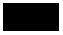 | 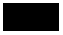 | 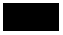 | 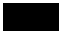 | 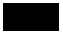 | 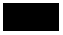 | 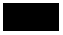 | 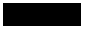 |
| 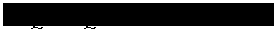 | 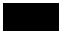 | 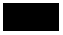 | 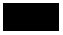 | 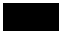 | 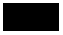 | 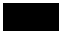 | 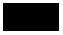 | 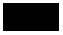 | 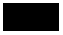 | 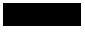 |
| 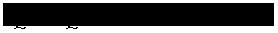 | 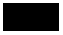 | 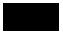 | 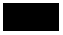 | 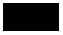 | 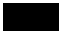 | 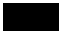 | 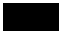 | 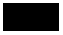 | 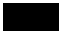 | 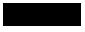 |
| 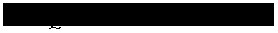 | 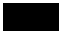 | 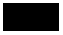 | 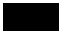 | 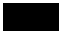 | 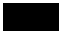 | 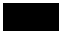 | 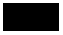 | 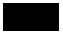 | 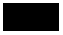 | 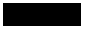 |
| 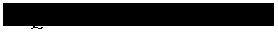 | 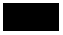 | 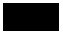 | 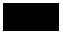 | 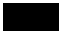 | 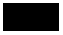 | 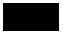 | 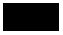 | 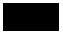 | 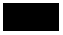 | 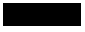 |
| 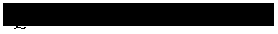 | 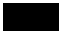 | 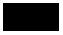 | 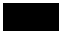 | 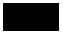 | 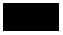 | 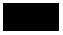 | 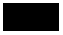 | 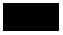 | 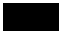 | 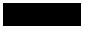 |
| 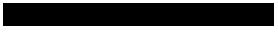 | 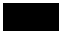 | 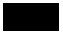 | 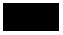 | 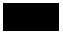 | 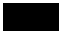 | 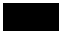 | 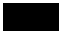 | 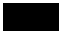 | 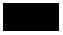 | 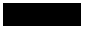 |
| 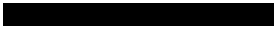 | 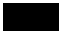 | 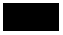 | 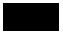 | 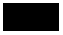 | 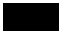 | 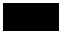 | 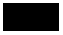 | 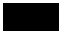 | 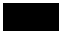 | 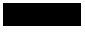 |
| 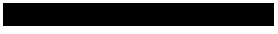 | 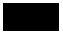 | 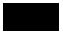 | 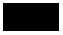 | 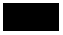 | 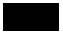 | 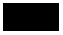 | 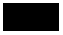 | 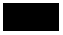 | 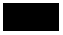 | 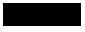 |
| LPKGFYAEGSRGGSQ | 0.2 | 0.1 | 0.2 | 0.1 | 0.6 | 0.6 | 0.4 | 0.5 | 0.2 | 1255 |
| PKGFYAEGSRGGSQA | 0.3 | 0.1 | 0.2 | 0.2 | 0.6 | 0.6 | 0.5 | 0.5 | 0.0 | 1256 |
| KGFYAEGSRGGSQAS | 0.2 | 0.1 | 0.2 | 0.2 | 0.6 | 0.5 | 0.4 | 0.5 | 0.2 | 1257 |
| GFYAEGSRGGSQASS | 0.3 | 0.1 | 0.2 | 0.4 | 0.7 | 0.5 | 0.4 | 0.4 | 0.1 | 1258 |
| FYAEGSRGGSQASSR | 0.3 | 0.2 | 0.2 | 0.3 | 0.7 | 0.8 | 0.5 | 0.6 | 0.4 | 1259 |
| YAEGSRGGSQASSRS | 0.3 | 0.1 | 0.3 | 0.4 | 0.7 | 0.7 | 0.5 | 0.6 | 0.6 | 1260 |
| AEGSRGGSQASSRSS | 0.3 | 0.2 | 0.3 | 0.3 | 0.7 | 0.7 | 0.5 | 0.6 | 0.4 | 1261 |
| EGSRGGSQASSRSSS | 0.3 | 0.2 | 0.3 | 0.4 | 0.9 | 0.8 | 0.5 | 0.6 | 0.6 | 1262 |
| GSRGGSQASSRSSSR | 0.3 | 0.1 | 0.3 | 0.4 | 0.7 | 0.8 | 0.5 | 0.8 | 0.2 | 1263 |
| SRGGSQASSRSSSRS | 0.3 | 0.1 | 0.3 | 0.4 | 0.6 | 0.7 | 0.4 | 0.9 | 0.1 | 1264 |
| RGGSQASSRSSSRSR | 0.3 | 0.1 | 0.3 | 0.4 | 0.7 | 0.7 | 0.5 | 0.7 | 0.3 | 1265 |
| GGSQASSRSSSRSRG | 0.3 | 0.1 | 0.3 | 0.4 | 0.7 | 0.7 | 0.5 | 0.9 | 0.7 | 1266 |
| GSQASSRSSSRSRGN | 0.3 | 0.1 | 0.4 | 0.4 | 0.7 | 0.6 | 0.6 | 0.7 | 0.3 | 1267 |
| SQASSRSSSRSRGNS | 0.2 | 0.1 | 0.2 | 0.4 | 0.6 | 0.6 | 0.4 | 0.6 | 0.2 | 1268 |
| QASSRSSSRSRGNSR | 0.2 | 0.1 | 0.3 | 0.3 | 0.6 | 0.6 | 0.5 | 0.6 | 0.2 | 1269 |
| ASSRSSSRSRGNSRN | 0.2 | 0.1 | 0.2 | 0.2 | 0.6 | 0.5 | 0.5 | 0.7 | 0.3 | 1270 |
| SSRSSSRSRGNSRNS | 0.2 | 0.1 | 0.2 | 0.2 | 0.5 | 0.6 | 0.5 | 0.7 | 0.3 | 1271 |
| SRSSSRSRGNSRNST | 0.2 | 0.1 | 0.2 | 0.2 | 0.4 | 0.6 | 0.4 | 0.7 | 0.2 | 1272 |
| RSSSRSRGNSRNSTP | 0.3 | 0.1 | 0.2 | 0.2 | 0.5 | 0.6 | 0.5 | 0.5 | 0.2 | 1273 |
| SSSRSRGNSRNSTPG | 0.4 | 0.2 | 0.2 | 0.1 | 0.5 | 0.5 | 0.5 | 0.6 | 0.2 | 1274 |
| SSRSRGNSRNSTPGS | 0.8 | 0.5 | 0.1 | 0.2 | 0.6 | 0.5 | 0.4 | 0.5 | 0.1 | 1275 |
| SRSRGNSRNSTPGSS | 1.0 | 0.6 | 0.3 | 0.2 | 0.7 | 0.7 | 0.5 | 0.6 | 0.4 | 1276 |
| RSRGNSRNSTPGSSR | 0.7 | 0.4 | 0.2 | 0.2 | 0.7 | 0.6 | 0.5 | 0.6 | 0.2 | 1277 |
| SRGNSRNSTPGSSRG | 0.7 | 0.4 | 0.2 | 0.2 | 0.7 | 0.7 | 0.5 | 0.7 | 0.4 | 1278 |
| RGNSRNSTPGSSRGN | 0.7 | 0.4 | 0.2 | 0.2 | 0.7 | 0.7 | 0.4 | 0.8 | 0.2 | 1279 |
| GNSRNSTPGSSRGNS | 0.7 | 0.5 | 0.2 | 0.2 | 0.7 | 0.7 | 0.5 | 0.7 | 0.2 | 1280 |
| NSRNSTPGSSRGNSP | 0.6 | 0.4 | 0.3 | 0.4 | 0.9 | 0.9 | 1.4 | 1.0 | 0.4 | 1281 |
| SRNSTPGSSRGNSPA | 0.7 | 0.5 | 0.4 | 0.2 | 0.8 | 0.9 | 0.6 | 0.7 | 0.4 | 1282 |
| RNSTPGSSRGNSPAR | 0.6 | 0.3 | 0.3 | 0.2 | 0.7 | 0.7 | 0.4 | 0.7 | 0.4 | 553 |
| NSTPGSSRGNSPARM | 0.7 | 0.5 | 0.4 | 0.6 | 1.2 | 1.2 | 1.6 | 1.5 | 1.8 | 554 |
| STPGSSRGNSPARMA | 0.5 | 0.3 | 0.4 | 0.3 | 0.7 | 0.8 | 0.5 | 0.7 | 0.8 | 555 |
| TPGSSRGNSPARMAS | 0.3 | 0.2 | 0.3 | 0.2 | 0.8 | 0.7 | 0.5 | 0.8 | 0.6 | 556 |
| PGSSRGNSPARMASG | 0.3 | 0.2 | 0.3 | 0.2 | 0.6 | 0.6 | 0.5 | 0.7 | 0.5 | 557 |
| GSSRGNSPARMASGG | 0.4 | 0.2 | 0.2 | 0.2 | 0.5 | 0.7 | 0.5 | 0.7 | 0.8 | 558 |
| SSRGNSPARMASGGG | 0.4 | 0.3 | 0.2 | 0.2 | 0.5 | 0.6 | 0.5 | 0.7 | 1.0 | 1283 |
| SRGNSPARMASGGGE | 0.3 | 0.2 | 0.1 | 0.2 | 0.6 | 0.6 | 0.6 | 0.6 | 0.2 | 1284 |
| RGNSPARMASGGGET | 0.3 | 0.1 | 0.2 | 0.2 | 0.5 | 0.5 | 0.5 | 0.5 | 0.1 | 1285 |
| GNSPARMASGGGETA | 0.3 | 0.1 | 0.2 | 0.2 | 0.6 | 0.6 | 0.6 | 0.5 | 0.2 | 1286 |
| 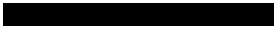 | 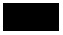 | 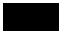 | 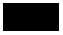 | 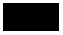 | 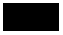 | 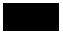 | 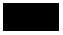 | 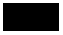 | 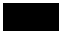 | 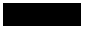 |
| 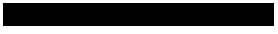 | 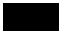 | 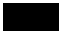 | 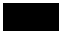 | 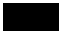 | 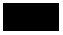 | 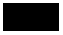 | 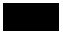 | 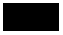 | 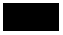 | 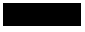 |
| 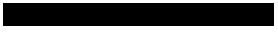 | 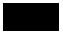 | 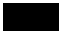 | 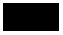 | 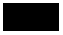 | 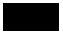 | 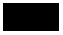 | 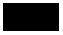 | 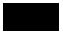 | 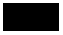 | 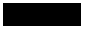 |
| 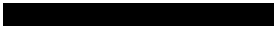 | 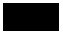 | 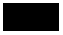 | 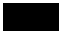 | 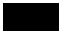 | 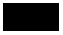 | 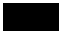 | 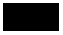 | 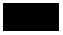 | 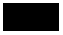 | 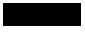 |
| 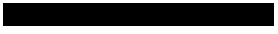 | 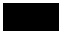 | 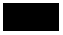 | 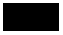 | 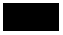 | 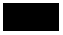 | 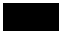 | 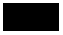 | 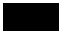 | 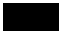 | 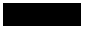 |
| 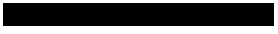 | 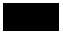 | 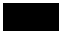 | 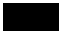 | 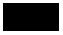 | 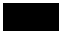 | 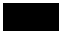 | 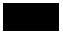 | 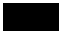 | 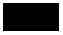 | 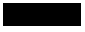 |
| 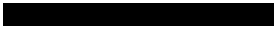 | 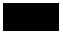 | 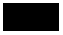 | 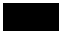 | 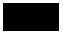 | 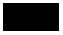 | 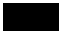 | 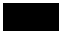 | 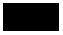 | 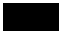 | 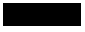 |
| SGGGETALALLLLDR | 0.3 | 0.1 | 0.2 | 0.2 | 0.7 | 0.7 | 0.4 | 0.5 | 0.1 | 1287 |
| GGGETALALLLLDRL | 0.2 | 0.1 | 0.1 | 0.1 | 0.7 | 0.5 | 0.4 | 0.6 | 0.2 | 1288 |
| GGETALALLLLDRLN | 0.2 | 0.1 | 0.2 | 0.2 | 0.6 | 0.6 | 0.4 | 0.5 | 0.4 | 1289 |
| GETALALLLLDRLNQ | 0.2 | 0.1 | 0.2 | 0.1 | 0.5 | 0.6 | 0.5 | 0.6 | 0.2 | 1290 |
| ETALALLLLDRLNQL | 0.2 | 0.1 | 0.1 | 0.1 | 0.6 | 0.4 | 0.5 | 0.5 | 0.3 | 1291 |
| TALALLLLDRLNQLE | 0.3 | 0.2 | 0.2 | 0.2 | 0.7 | 0.7 | 0.5 | 0.6 | 0.8 | 1292 |
| ALALLLLDRLNQLES | 0.3 | 0.2 | 0.1 | 0.2 | 0.5 | 0.6 | 0.5 | 0.5 | 0.4 | 1293 |
| LALLLLDRLNQLESK | 0.2 | 0.1 | 0.1 | 0.1 | 0.5 | 0.5 | 0.7 | 0.5 | 0.4 | 1294 |
| ALLLLDRLNQLESKV | 0.2 | 0.1 | 0.2 | 0.1 | 0.7 | 0.4 | 0.6 | 0.5 | 0.2 | 1295 |
| LLLLDRLNQLESKVS | 0.2 | 0.1 | 0.2 | 0.1 | 0.7 | 0.4 | 0.7 | 0.4 | 0.4 | 1296 |
| LLLDRLNQLESKVSG | 0.3 | 0.2 | 0.4 | 0.2 | 0.8 | 0.6 | 0.9 | 0.7 | 1.1 | 1297 |
| LLDRLNQLESKVSGK | 0.2 | 0.1 | 0.3 | 0.2 | 0.7 | 0.6 | 0.5 | 0.7 | 0.4 | 1298 |
| LDRLNQLESKVSGKG | 0.3 | 0.2 | 0.3 | 0.3 | 0.5 | 0.6 | 0.5 | 0.7 | 0.8 | 1299 |
| DRLNQLESKVSGKGQ | 0.3 | 0.1 | 0.3 | 0.2 | 0.8 | 0.7 | 0.5 | 0.7 | 0.5 | 1300 |
| RLNQLESKVSGKGQQ | 0.3 | 0.2 | 0.3 | 0.2 | 0.7 | 0.9 | 0.6 | 0.7 | 0.4 | 1301 |
| LNQLESKVSGKGQQQ | 0.3 | 0.1 | 0.3 | 0.2 | 0.7 | 0.9 | 0.6 | 0.7 | 0.4 | 1302 |
| NQLESKVSGKGQQQQ | 0.3 | 0.2 | 0.3 | 0.2 | 0.7 | 0.8 | 0.6 | 0.7 | 0.5 | 1303 |
| QLESKVSGKGQQQQG | 0.3 | 0.1 | 0.3 | 0.2 | 0.7 | 0.8 | 0.6 | 0.8 | 0.5 | 1304 |
| LESKVSGKGQQQQGQ | 0.3 | 0.1 | 0.2 | 0.1 | 0.6 | 0.6 | 0.5 | 0.7 | 0.4 | 1305 |
| ESKVSGKGQQQQGQT | 0.2 | 0.1 | 0.2 | 0.2 | 0.6 | 0.7 | 0.5 | 0.7 | 0.4 | 1306 |
| SKVSGKGQQQQGQTV | 0.2 | 0.1 | 0.3 | 0.1 | 0.5 | 0.6 | 0.5 | 0.7 | 0.3 | 1307 |
| KVSGKGQQQQGQTVT | 0.3 | 0.1 | 0.3 | 0.2 | 0.6 | 0.6 | 0.5 | 0.6 | 0.6 | 1308 |
| VSGKGQQQQGQTVTK | 1.2 | 0.6 | 0.6 | 0.4 | 0.9 | 1.2 | 0.6 | 1.0 | 0.5 | 1309 |
| SGKGQQQQGQTVTKK | 0.3 | 0.1 | 0.2 | 0.4 | 0.7 | 0.9 | 0.6 | 0.7 | 0.5 | 1310 |
| GKGQQQQGQTVTKKS | 0.3 | 0.1 | 0.3 | 0.3 | 0.7 | 0.7 | 0.5 | 0.7 | 0.2 | 1311 |
| KGQQQQGQTVTKKSA | 0.3 | 0.1 | 0.2 | 0.3 | 0.7 | 0.8 | 0.6 | 0.6 | 0.4 | 1312 |
| GQQQQGQTVTKKSAA | 0.3 | 0.1 | 0.3 | 0.3 | 0.8 | 0.8 | 0.5 | 0.5 | 0.1 | 1313 |
| QQQQGQTVTKKSAAE | 0.8 | 0.3 | 0.7 | 0.2 | 0.7 | 0.7 | 0.7 | 0.9 | 0.3 | 1314 |
| QQQGQTVTKKSAAEA | 0.6 | 0.2 | 0.5 | 0.2 | 0.5 | 0.5 | 0.6 | 0.6 | 0.3 | 1315 |
| 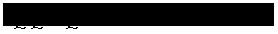 | 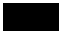 | 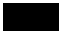 | 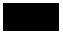 | 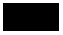 | 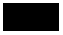 | 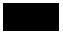 | 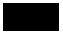 | 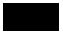 | 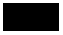 | 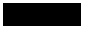 |
| 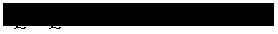 | 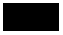 | 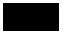 | 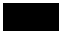 | 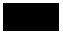 | 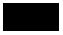 | 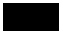 | 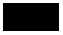 | 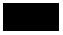 | 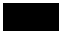 | 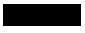 |
| 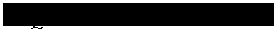 | 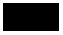 | 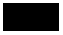 | 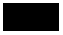 | 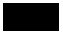 | 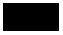 | 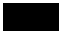 | 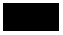 | 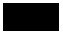 | 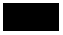 | 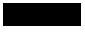 |
| 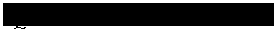 | 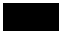 | 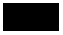 | 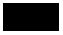 | 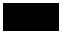 | 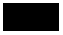 | 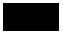 | 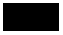 | 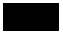 | 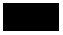 | 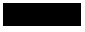 |
| 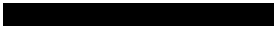 | 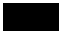 | 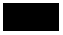 | 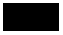 | 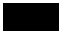 | 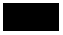 | 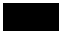 | 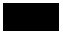 | 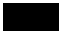 | 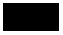 | 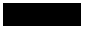 |
| 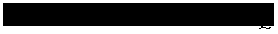 | 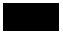 | 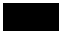 | 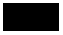 | 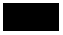 | 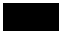 | 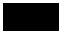 | 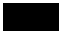 | 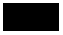 | 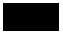 | 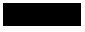 |
| 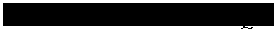 | 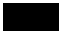 | 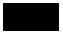 | 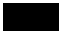 | 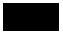 | 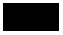 | 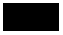 | 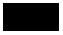 | 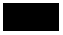 | 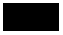 | 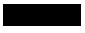 |
| 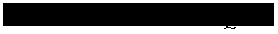 | 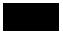 | 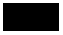 | 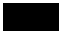 | 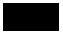 | 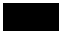 | 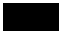 | 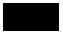 | 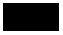 | 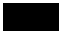 | 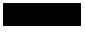 |
| 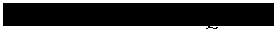 | 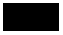 | 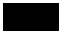 | 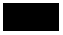 | 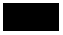 | 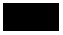 | 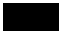 | 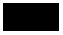 | 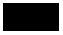 | 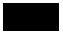 | 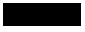 |
| 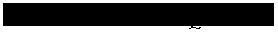 | 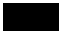 | 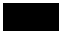 | 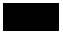 | 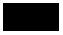 | 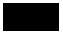 | 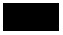 | 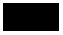 | 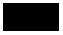 | 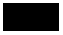 | 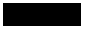 |
| 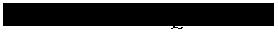 | 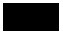 | 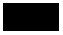 | 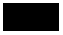 | 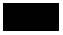 | 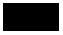 | 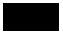 | 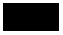 | 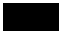 | 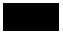 | 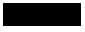 |
| AEASKKPRQKRTATK | 0.2 | 0.1 | 0.2 | 0.3 | 0.7 | 0.7 | 0.4 | 0.7 | 0.4 | 1316 |
| EASKKPRQKRTATKQ | 0.2 | 0.1 | 0.3 | 0.3 | 0.7 | 0.7 | 0.5 | 0.6 | 0.3 | 1317 |
| ASKKPRQKRTATKQY | 0.2 | 0.1 | 0.3 | 0.2 | 0.7 | 0.6 | 0.5 | 0.5 | 0.3 | 1318 |
| SKKPRQKRTATKQYN | 0.2 | 0.1 | 0.3 | 0.2 | 0.6 | 0.7 | 0.5 | 0.4 | 0.1 | 1319 |
| KKPRQKRTATKQYNV | 0.2 | 0.1 | 0.3 | 0.2 | 0.8 | 0.8 | 0.7 | 0.6 | 0.1 | 1320 |
| 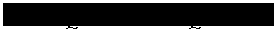 | 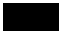 | 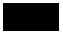 | 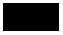 | 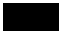 | 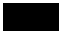 | 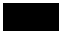 | 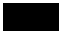 | 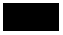 | 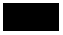 | 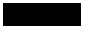 |
| 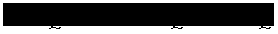 | 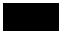 | 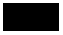 | 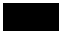 | 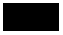 | 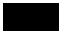 | 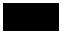 | 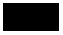 | 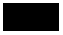 | 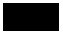 | 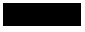 |
| 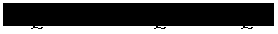 | 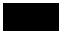 | 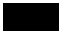 | 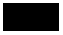 | 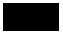 | 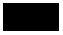 | 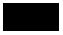 | 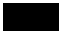 | 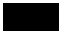 | 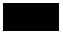 | 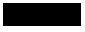 |
| 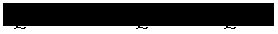 | 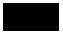 | 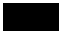 | 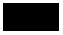 | 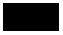 | 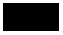 | 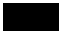 | 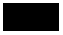 | 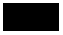 | 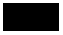 | 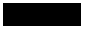 |
| 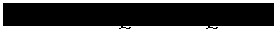 | 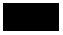 | 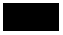 | 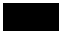 | 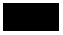 | 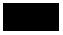 | 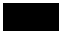 | 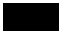 | 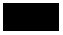 | 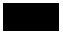 | 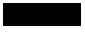 |
| 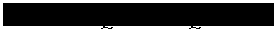 | 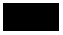 | 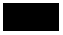 | 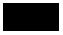 | 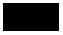 | 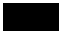 | 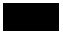 | 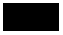 | 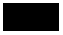 | 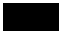 | 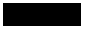 |
| 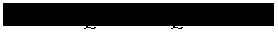 | 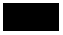 | 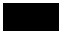 | 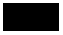 | 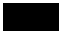 | 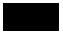 | 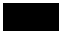 | 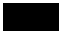 | 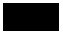 | 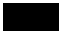 | 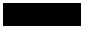 |
| ATKQYNVTQAFGRRG | 0.3 | 0.2 | 0.2 | 0.2 | 0.7 | 0.6 | 0.5 | 0.8 | 0.3 | 565 |
| TKQYNVTQAFGRRGP | 0.3 | 0.1 | 0.2 | 0.2 | 0.8 | 0.7 | 0.5 | 0.7 | 0.5 | 566 |
| KQYNVTQAFGRRGPE | 0.3 | 0.1 | 0.1 | 0.2 | 0.6 | 0.7 | 0.5 | 0.6 | 0.3 | 567 |
| QYNVTQAFGRRGPEQ | 0.3 | 0.1 | 0.1 | 0.2 | 0.6 | 0.6 | 0.4 | 0.6 | 0.5 | 568 |
| YNVTQAFGRRGPEQT | 0.3 | 0.1 | 0.2 | 0.2 | 0.7 | 0.7 | 0.5 | 0.6 | 0.6 | 569 |
| NVTQAFGRRGPEQTQ | 0.2 | 0.1 | 0.2 | 0.2 | 0.6 | 0.7 | 0.5 | 0.6 | 0.5 | 570 |
| VTQAFGRRGPEQTQG | 0.3 | 0.1 | 0.1 | 0.2 | 0.6 | 0.5 | 0.5 | 0.7 | 0.5 | 571 |
| TQAFGRRGPEQTQGN | 0.2 | 0.1 | 0.2 | 0.2 | 0.6 | 0.5 | 0.5 | 0.5 | 0.3 | 572 |
| QAFGRRGPEQTQGNF | 0.3 | 0.1 | 0.2 | 0.2 | 0.6 | 0.8 | 0.7 | 0.4 | 0.2 | 1321 |
| AFGRRGPEQTQGNFG | 0.2 | 0.1 | 0.2 | 0.3 | 0.5 | 0.7 | 0.8 | 0.6 | 0.4 | 1322 |
| 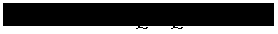 | 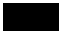 | 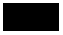 | 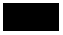 | 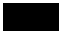 | 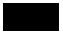 | 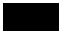 | 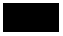 | 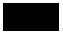 | 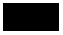 | 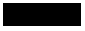 |
| 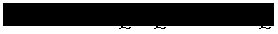 | 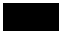 | 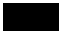 | 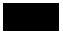 | 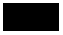 | 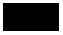 | 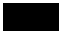 | 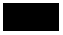 | 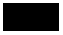 | 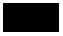 | 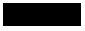 |
| 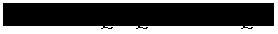 | 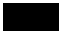 | 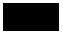 | 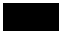 | 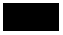 | 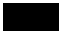 | 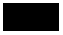 | 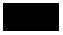 | 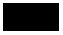 | 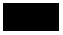 | 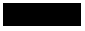 |
| 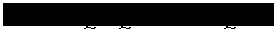 | 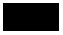 | 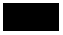 | 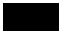 | 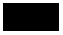 | 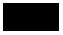 | 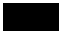 | 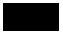 | 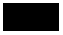 | 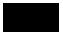 | 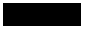 |
| 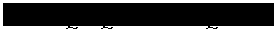 | 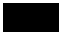 | 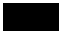 | 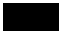 | 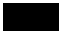 | 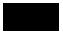 | 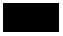 | 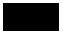 | 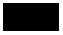 | 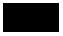 | 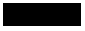 |
| 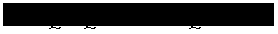 | 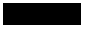 | 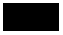 | 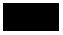 | 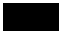 | 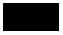 | 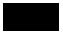 | 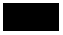 | 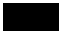 | 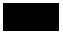 | 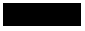 |
| 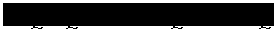 | 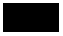 | 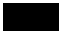 | 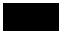 | 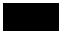 | 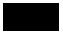 | 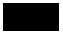 | 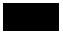 | 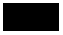 | 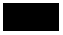 | 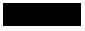 |
| 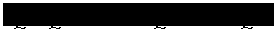 | 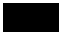 | 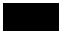 | 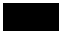 | 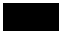 | 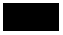 | 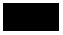 | 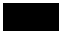 | 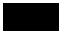 | 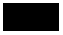 | 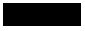 |
| TQGNFGDQDLIRQGT | 0.3 | 0.2 | 0.2 | 0.2 | 0.9 | 0.8 | 0.6 | 0.6 | 0.6 | 1323 |
| QGNFGDQDLIRQGTD | 0.3 | 0.2 | 0.2 | 0.2 | 0.8 | 0.8 | 0.5 | 0.8 | 0.6 | 1324 |
| GNFGDQDLIRQGTDY | 0.3 | 0.2 | 0.2 | 0.2 | 0.8 | 0.7 | 0.6 | 0.8 | 0.5 | 1325 |
| NFGDQDLIRQGTDYK | 0.3 | 0.2 | 0.2 | 0.2 | 0.7 | 0.7 | 0.6 | 0.6 | 0.8 | 1326 |
| FGDQDLIRQGTDYKH | 0.3 | 0.2 | 0.2 | 0.2 | 0.7 | 0.7 | 0.6 | 0.6 | 0.7 | 1327 |
| GDQDLIRQGTDYKHW | 0.2 | 0.1 | 0.2 | 0.3 | 0.6 | 0.7 | 0.9 | 0.7 | 0.5 | 1328 |
| DQDLIRQGTDYKHWP | 0.3 | 0.1 | 0.2 | 0.2 | 0.7 | 0.6 | 0.8 | 0.7 | 0.5 | 1329 |
| QDLIRQGTDYKHWPQ | 0.2 | 0.1 | 0.2 | 0.2 | 0.6 | 0.5 | 0.4 | 0.4 | 0.2 | 1330 |
| DLIRQGTDYKHWPQI | 0.2 | 0.1 | 0.2 | 0.2 | 0.6 | 0.6 | 0.9 | 0.6 | 0.4 | 1331 |
| LIRQGTDYKHWPQIA | 0.3 | 0.2 | 0.4 | 0.2 | 0.8 | 0.9 | 0.9 | 0.6 | 0.4 | 1332 |
| IRQGTDYKHWPQIAQ | 0.2 | 0.1 | 0.2 | 0.2 | 0.7 | 0.6 | 0.9 | 0.6 | 0.3 | 1333 |
| RQGTDYKHWPQIAQF | 0.3 | 0.2 | 0.2 | 0.2 | 0.7 | 0.8 | 0.7 | 0.6 | 0.3 | 1334 |
| QGTDYKHWPQIAQFA | 0.3 | 0.2 | 0.2 | 0.2 | 0.8 | 0.8 | 0.7 | 0.7 | 0.2 | 1335 |
| GTDYKHWPQIAQFAP | 0.3 | 0.1 | 0.2 | 0.2 | 0.7 | 0.8 | 0.6 | 0.7 | 0.6 | 1336 |
| TDYKHWPQIAQFAPS | 0.3 | 0.2 | 0.2 | 0.2 | 0.8 | 0.9 | 0.7 | 0.7 | 0.4 | 1337 |
| DYKHWPQIAQFAPSA | 0.3 | 0.2 | 0.2 | 0.2 | 0.9 | 0.9 | 0.7 | 0.8 | 0.4 | 1338 |
| YKHWPQIAQFAPSAS | 0.3 | 0.1 | 0.2 | 0.2 | 0.8 | 0.7 | 0.6 | 0.7 | 0.4 | 1339 |
| KHWPQIAQFAPSASA | 0.3 | 0.1 | 0.3 | 0.2 | 0.8 | 0.7 | 0.7 | 0.8 | 0.6 | 1340 |
| HWPQIAQFAPSASAF | 0.3 | 0.2 | 0.2 | 0.2 | 0.8 | 0.7 | 0.6 | 0.8 | 0.4 | 1341 |
| WPQIAQFAPSASAFF | 0.2 | 0.1 | 0.1 | 0.2 | 0.6 | 0.5 | 0.5 | 0.7 | 0.3 | 1342 |
| PQIAQFAPSASAFFG | 0.2 | 0.1 | 0.2 | 0.1 | 0.6 | 0.6 | 0.5 | 0.6 | 0.6 | 1343 |
| QIAQFAPSASAFFGM | 0.3 | 0.1 | 0.2 | 0.2 | 0.7 | 0.6 | 0.5 | 0.7 | 0.7 | 1344 |
| IAQFAPSASAFFGMS | 0.2 | 0.1 | 0.1 | 0.1 | 0.6 | 0.5 | 0.4 | 0.6 | 0.2 | 1345 |
| AQFAPSASAFFGMSR | 0.2 | 0.1 | 0.1 | 0.1 | 0.5 | 0.4 | 0.4 | 0.6 | 0.1 | 1346 |
| QFAPSASAFFGMSRI | 0.2 | 0.1 | 0.1 | 0.1 | 0.4 | 0.4 | 0.4 | 0.5 | 0.1 | 1347 |
| FAPSASAFFGMSRIG | 0.2 | 0.1 | 0.2 | 0.1 | 0.6 | 0.5 | 0.4 | 0.5 | 0.4 | 1348 |
| APSASAFFGMSRIGM | 0.2 | 0.1 | 0.2 | 0.1 | 0.5 | 0.5 | 0.6 | 0.4 | 0.2 | 1349 |
| PSASAFFGMSRIGME | 0.2 | 0.1 | 0.1 | 0.1 | 0.6 | 0.5 | 0.5 | 0.6 | 0.1 | 1350 |
| SASAFFGMSRTGMEV | 0.3 | 0.2 | 0.2 | 0.2 | 0.7 | 0.6 | 0.5 | 0.7 | 0.4 | 1351 |
| ASAFFGMSRIGMEVT | 0.2 | 0.3 | 0.2 | 0.2 | 0.7 | 0.7 | 0.8 | 0.7 | 0.3 | 1352 |
| SAFFGMSRIGMEVTP | 0.3 | 0.2 | 0.2 | 0.2 | 0.7 | 0.8 | 0.6 | 0.7 | 0.3 | 1353 |
| AFFGMSRIGMEVTPS | 0.3 | 0.1 | 0.2 | 0.2 | 0.6 | 0.7 | 0.5 | 0.5 | 0.2 | 1354 |
| FFGMSRIGMEVTPSG | 0.3 | 0.1 | 0.2 | 0.2 | 0.7 | 0.7 | 0.6 | 0.8 | 0.3 | 1355 |
| FGMSRIGMEVTPSGT | 0.3 | 0.1 | 0.2 | 0.2 | 0.8 | 0.7 | 0.6 | 0.7 | 0.3 | 1356 |
| GMSRIGMEVTPSGTW | 0.3 | 0.1 | 0.2 | 0.2 | 0.7 | 0.7 | 0.5 | 0.7 | 0.5 | 1357 |
| MSRIGMEVTPSGTWL | 0.3 | 0.1 | 0.2 | 0.2 | 0.6 | 0.7 | 0.5 | 0.6 | 0.3 | 1358 |
| SRIGMEVTPSGTWLT | 0.2 | 0.1 | 0.2 | 0.2 | 0.7 | 0.6 | 0.5 | 0.6 | 0.4 | 1359 |
| RIGMEVTPSGTWLTY | 0.2 | 0.1 | 0.1 | 0.1 | 0.4 | 0.4 | 0.3 | 0.9 | 0.5 | 1360 |
| IGMEVTPSGTWLTYH | 0.2 | 0.1 | 0.1 | 0.2 | 0.6 | 0.6 | 0.5 | 0.5 | 0.3 | 1361 |
| GMEVTPSGTWLTYHG | 0.3 | 0.1 | 0.1 | 0.2 | 0.5 | 0.6 | 0.5 | 0.7 | 0.2 | 1362 |
| MEVTPSGTWLTYHGA | 0.3 | 0.1 | 0.1 | 0.2 | 0.6 | 0.5 | 0.5 | 0.7 | 0.2 | 1363 |
| EVTPSGTWLTYHGAI | 0.2 | 0.1 | 0.1 | 0.2 | 0.7 | 0.5 | 0.4 | 0.5 | 0.1 | 1364 |
| VTPSGTWLTYHGAIK | 0.3 | 0.1 | 0.2 | 0.2 | 0.7 | 0.8 | 0.7 | 0.5 | 0.5 | 1365 |
| TPSGTWLTYHGAIKL | 0.2 | 0.1 | 0.2 | 0.2 | 0.7 | 0.6 | 0.7 | 0.5 | 0.3 | 1366 |
| PSGTWLTYHGAIKLD | 0.3 | 0.2 | 0.2 | 0.2 | 1.0 | 0.8 | 0.7 | 0.7 | 0.6 | 1367 |
| SGTWLTYHGAIKLDD | 0.2 | 0.1 | 0.1 | 0.1 | 0.7 | 0.5 | 0.6 | 0.6 | 0.5 | 1368 |
| GTWLTYHGAIKLDDK | 0.2 | 0.1 | 0.2 | 0.1 | 0.7 | 0.7 | 0.5 | 0.7 | 0.4 | 1369 |
| TWLTYHGAIKLDDKD | 0.2 | 0.1 | 0.1 | 0.2 | 0.6 | 0.6 | 0.5 | 0.6 | 0.3 | 1370 |
| WLTYHGAIKLDDKDP | 0.3 | 0.2 | 0.2 | 0.2 | 0.7 | 0.8 | 0.6 | 0.7 | 0.3 | 1371 |
| LTYHGAIKLDDKDPQ | 0.2 | 0.1 | 0.2 | 0.2 | 0.6 | 1.0 | 0.5 | 0.7 | 0.2 | 1372 |
| TYHGAIKLDDKDPQF | 0.3 | 0.1 | 0.1 | 0.2 | 0.8 | 1.1 | 0.6 | 0.7 | 0.4 | 1373 |
| YHGAIKLDDKDPQFK | 0.3 | 0.2 | 0.3 | 0.2 | 0.8 | 1.6 | 0.7 | 0.7 | 0.9 | 1374 |
| HGAIKLDDKDPQFKD | 0.2 | 0.1 | 0.2 | 0.1 | 0.8 | 0.7 | 0.6 | 0.6 | 0.4 | 1375 |
| GAIKLDDKDPQFKDN | 0.2 | 0.1 | 0.2 | 0.2 | 0.7 | 0.9 | 0.6 | 0.7 | 0.5 | 1376 |
| AIKLDDKDPQFKDNV | 0.2 | 0.1 | 0.2 | 0.2 | 0.8 | 0.9 | 0.6 | 0.6 | 0.7 | 1377 |
| 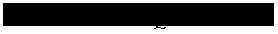 | 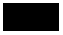 | 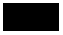 | 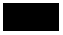 | 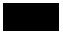 | 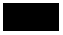 | 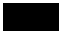 | 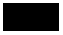 | 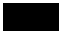 | 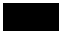 | 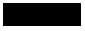 |
| 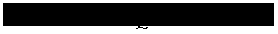 | 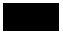 | 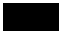 | 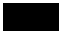 | 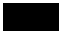 | 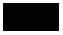 | 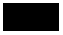 | 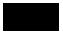 | 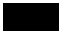 | 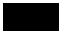 | 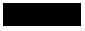 |
| 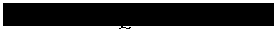 | 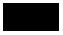 | 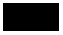 | 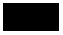 | 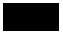 | 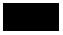 | 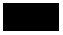 | 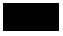 | 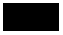 | 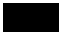 | 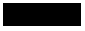 |
| 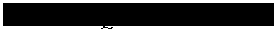 | 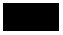 | 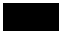 | 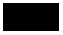 | 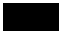 | 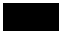 | 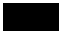 | 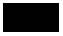 | 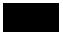 | 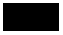 | 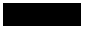 |
| 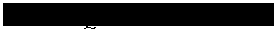 | 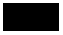 | 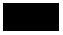 | 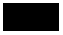 | 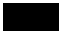 | 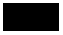 | 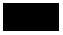 | 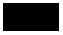 | 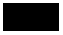 | 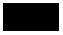 | 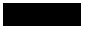 |
| 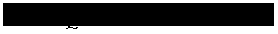 | 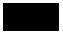 | 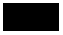 | 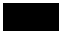 | 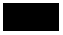 | 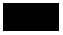 | 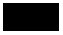 | 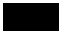 | 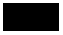 | 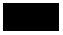 | 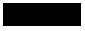 |
| 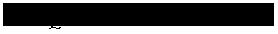 | 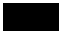 | 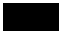 | 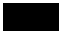 | 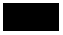 | 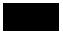 | 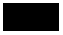 | 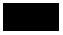 | 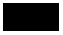 | 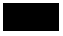 | 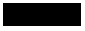 |
| 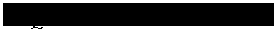 | 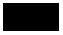 | 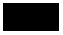 | 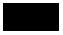 | 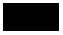 | 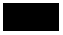 | 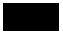 | 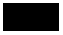 | 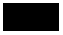 | 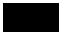 | 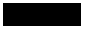 |
| 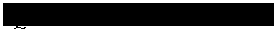 | 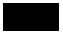 | 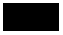 | 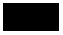 | 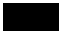 | 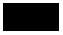 | 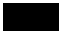 | 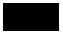 | 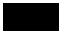 | 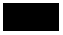 | 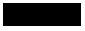 |
| FKDNVILLNKHIDAY | 0.2 | 0.1 | 0.2 | 0.2 | 0.6 | 0.6 | 0.5 | 0.7 | 0.2 | 1378 |
| KDNVILLNKHIDAYK | 0.3 | 0.2 | 0.3 | 0.3 | 0.7 | 0.8 | 0.6 | 0.8 | 0.4 | 1379 |
| DNVILLNKHIDAYKT | 0.2 | 0.1 | 0.2 | 0.1 | 0.6 | 0.6 | 0.5 | 0.6 | 0.2 | 1380 |
| NVILLNKHIDAYKTF | 0.2 | 0.1 | 0.2 | 0.1 | 0.7 | 0.6 | 0.5 | 0.6 | 0.3 | 1381 |
| VILLNKHIDAYKTFP | 0.2 | 0.1 | 0.2 | 0.2 | 0.7 | 0.7 | 0.5 | 0.5 | 0.4 | 1382 |
| ILLNKHIDAYKTFPP | 0.2 | 0.1 | 0.2 | 0.2 | 0.7 | 0.7 | 0.5 | 0.5 | 0.3 | 1383 |
| LLNKHIDAYKTFPPT | 0.2 | 0.1 | 0.2 | 0.2 | 0.7 | 0.6 | 0.5 | 0.6 | 0.5 | 1384 |
| LNKHIDAYKTFPPTE | 0.2 | 0.1 | 0.1 | 0.1 | 0.7 | 0.5 | 0.4 | 0.5 | 0.4 | 1385 |
| NKHIDAYKTFPPTEP | 0.2 | 0.1 | 0.1 | 0.2 | 0.6 | 0.5 | 0.5 | 0.6 | 0.3 | 1386 |
| KHTDAYKTFPPTEPK | 0.3 | 0.1 | 0.3 | 0.2 | 0.7 | 0.8 | 0.5 | 0.8 | 0.5 | 1387 |
| HIDAYKTFPPTEPKK | 0.2 | 0.1 | 0.2 | 0.2 | 0.6 | 0.6 | 0.4 | 0.5 | 0.2 | 1388 |
| IDAYKTFPPTEPKKD | 0.3 | 0.1 | 0.3 | 0.2 | 0.7 | 0.9 | 0.4 | 0.6 | 0.4 | 1389 |
| DAYKTFPPTEPKKDK | 0.3 | 0.2 | 0.3 | 0.3 | 0.8 | 1.1 | 0.6 | 0.7 | 0.4 | 1390 |
| AYKTFPPTEPKKDKK | 0.2 | 0.1 | 0.3 | 0.2 | 0.7 | 0.8 | 0.6 | 0.7 | 0.3 | 1391 |
| YKTFPPTEPKKDKKK | 0.2 | 0.1 | 0.5 | 0.2 | 0.7 | 0.7 | 0.5 | 0.8 | 0.2 | 1392 |
| KTFPPTEPKKDKKKK | 0.2 | 0.1 | 0.4 | 0.2 | 0.8 | 0.6 | 0.4 | 0.8 | 0.1 | 1393 |
| TFPPTEPKKDKKKKT | 0.2 | 0.1 | 0.9 | 0.3 | 0.8 | 0.8 | 0.5 | 0.9 | 0.3 | 1394 |
| FPPTEPKKDKKKKTD | 0.2 | 0.1 | 1.3 | 0.2 | 0.6 | 0.7 | 0.6 | 0.6 | 0.1 | 1395 |
| PPTEPKKDKKKKTDE | 0.2 | 0.1 | 2.0 | 0.2 | 0.6 | 0.7 | 0.5 | 0.6 | 0.3 | 1396 |
| PTEPKKDKKKKTDEA | 0.2 | 0.1 | 2.0 | 0.2 | 0.6 | 0.7 | 0.5 | 0.5 | 0.2 | 1397 |
| TEPKKDKKKKTDEAQ | 0.2 | 0.1 | 2.5 | 0.2 | 0.7 | 0.7 | 0.6 | 0.6 | 0.5 | 1398 |
| EPKKDKKKKTDEAQP | 0.2 | 0.1 | 2.4 | 0.2 | 0.7 | 0.7 | 0.7 | 0.6 | 0.5 | 1399 |
| PKKDKKKKTDEAQPL | 0.2 | 0.1 | 2.5 | 0.2 | 0.7 | 0.7 | 0.5 | 0.5 | 0.1 | 1400 |
| KKDKKKKTDEAQPLP | 0.3 | 0.2 | 1.9 | 0.2 | 0.7 | 0.7 | 0.6 | 0.6 | 0.4 | 1401 |
| KDKKKKTDEAQPLPQ | 0.2 | 0.1 | 0.2 | 0.2 | 0.7 | 0.6 | 0.6 | 0.5 | 0.5 | 1402 |
| DKKKKTDEAQPLPQR | 0.2 | 0.1 | 0.2 | 0.2 | 0.6 | 0.6 | 0.5 | 0.6 | 0.5 | 1403 |
| KKKKTDEAQPLPQRQ | 0.3 | 0.1 | 0.3 | 0.3 | 0.7 | 0.7 | 0.8 | 0.9 | 0.6 | 1404 |
| KKKTDEAQPLPQRQK | 0.2 | 0.1 | 0.2 | 0.7 | 0.9 | 0.6 | 1.1 | 1.0 | 0.4 | 1405 |
| KKTDEAQPLPQRQKK | 0.2 | 0.1 | 0.3 | 0.4 | 0.7 | 0.6 | 1.7 | 1.1 | 0.2 | 1406 |
| KTDEAQPLPQRQKKQ | 0.3 | 0.2 | 0.3 | 0.3 | 0.9 | 0.9 | 1.4 | 1.5 | 0.6 | 1407 |
| TDEAQPLPQRQKKQP | 0.2 | 0.2 | 0.3 | 0.2 | 0.7 | 0.7 | 1.2 | 1.0 | 0.7 | 1408 |
| DEAQPLPQRQKKQPT | 0.2 | 0.2 | 0.3 | 0.3 | 0.8 | 0.7 | 1.5 | 1.6 | 0.6 | 1409 |
| EAQPLPQRQKKQPTV | 0.2 | 0.2 | 0.3 | 0.3 | 0.7 | 0.7 | 1.2 | 1.3 | 0.4 | 1410 |
| AQPLPQRQKKQPTVT | 0.3 | 0.2 | 0.4 | 0.4 | 0.7 | 0.8 | 1.6 | 1.4 | 0.3 | 1411 |
| QPLPQRQKKQPTVTL | 0.2 | 0.2 | 0.3 | 0.4 | 0.6 | 0.6 | 1.3 | 1.3 | 0.2 | 414 |
| PLPQRQKKQPTVTLL | 0.2 | 0.1 | 0.2 | 0.2 | 0.5 | 0.6 | 1.0 | 1.0 | 0.2 | 415 |
| LPQRQKKQPTVTLLP | 0.2 | 0.2 | 0.3 | 0.1 | 0.7 | 0.7 | 0.5 | 0.7 | 0.4 | 416 |
| PQRQKKQPTVTLLPA | 0.3 | 0.2 | 0.3 | 0.2 | 0.7 | 0.7 | 0.5 | 0.6 | 0.5 | 417 |
| QRQKKQPTVTLLPAA | 0.3 | 0.1 | 0.3 | 0.2 | 0.7 | 0.8 | 0.6 | 0.6 | 0.7 | 418 |
| RQKKQPTVTLLPAAD | 0.3 | 0.2 | 0.2 | 0.2 | 0.7 | 0.8 | 0.6 | 0.6 | 0.6 | 419 |
| QKKQPTVTLLPAADM | 0.3 | 0.2 | 0.2 | 0.2 | 0.7 | 0.6 | 0.6 | 0.5 | 0.4 | 420 |
| KKQPTVTLLPAADMD | 0.3 | 0.2 | 0.2 | 0.2 | 1.0 | 0.7 | 0.6 | 0.5 | 1.3 | 1412 |
| KQPTVTLLPAADMDD | 0.3 | 0.2 | 0.2 | 0.2 | 1.2 | 0.8 | 0.6 | 1.1 | 2.4 | 1413 |
| QPTVTLLPAADMDDF | 0.3 | 0.2 | 0.2 | 0.2 | 1.1 | 0.7 | 0.5 | 0.7 | 1.9 | 1414 |
| PTVTLLPAADMDDFS | 0.3 | 0.2 | 0.2 | 0.1 | 1.4 | 0.9 | 0.6 | 0.7 | 1.9 | 1415 |
| TVTLLPAADMDDFSR | 0.2 | 0.1 | 0.1 | 0.2 | 0.7 | 0.5 | 0.5 | 0.5 | 0.3 | 1416 |
| VTLLPAADMDDFSRQ | 0.3 | 0.1 | 0.2 | 0.1 | 0.6 | 0.6 | 0.6 | 0.6 | 0.3 | 1417 |
| TLLPAADMDDFSRQL | 0.3 | 0.2 | 0.2 | 0.4 | 1.0 | 0.7 | 0.6 | 0.7 | 0.7 | 1418 |
| LLPAADMDDFSRQLQ | 0.3 | 0.2 | 0.3 | 0.3 | 0.8 | 0.6 | 0.6 | 0.6 | 0.4 | 1419 |
| LPAADMDDFSRQLQN | 0.2 | 0.2 | 0.4 | 0.3 | 0.7 | 0.6 | 0.5 | 0.6 | 0.2 | 1420 |
| PAADMDDFSRQLQNS | 0.3 | 0.2 | 0.2 | 0.6 | 0.8 | 0.7 | 0.6 | 0.5 | 0.3 | 1421 |
| AADMDDFSRQLQNSM | 0.3 | 0.2 | 0.2 | 1.2 | 0.7 | 0.6 | 0.7 | 0.7 | 0.4 | 1422 |
| ADMDDFSRQLQNSMS | 0.3 | 0.2 | 0.2 | 1.5 | 0.7 | 0.6 | 0.8 | 0.7 | 0.3 | 1423 |
| DMDDFSRQLQNSMSG | 0.2 | 0.2 | 0.2 | 0.3 | 0.8 | 0.7 | 0.7 | 0.7 | 0.3 | 1424 |
| MDDFSRQLQNSMSGA | 0.2 | 0.1 | 0.3 | 0.2 | 0.8 | 0.7 | 0.8 | 0.7 | 0.6 | 1425 |
| DDFSRQLQNSMSGAS | 0.2 | 0.1 | 0.2 | 0.2 | 0.8 | 0.7 | 0.7 | 0.6 | 0.4 | 1426 |
| DFSRQLQNSMSGASA | 0.2 | 0.1 | 0.2 | 0.2 | 0.7 | 0.6 | 0.5 | 0.5 | 0.3 | 1427 |
| FSRQLQNSMSGASAD | 0.2 | 0.1 | 0.1 | 0.2 | 0.7 | 0.6 | 0.6 | 0.4 | 0.6 | 1428 |
| SRQLQNSMSGASADS | 0.2 | 0.1 | 0.2 | 0.2 | 0.8 | 0.6 | 0.6 | 0.5 | 0.5 | 1429 |
| RQLQNSMSGASADST | 0.2 | 0.2 | 0.2 | 0.2 | 0.7 | 0.6 | 0.6 | 0.5 | 0.3 | 1430 |
| QLQNSMSGASADSTQ | 0.2 | 0.1 | 0.2 | 0.1 | 0.7 | 0.5 | 0.6 | 0.6 | 0.3 | 1431 |
| LQNSMSGASADSTQA | 0.2 | 0.1 | 0.1 | 0.2 | 0.6 | 0.4 | 0.6 | 0.6 | 0.4 | 1432 |

TABLE 16
Binding of the sera called SARS-yellow, SARS-green, 1a,
1b, 2, 6, 37, 62 and London to looped/cyclic peptides of protein N
of SARS-CoV Urbani.

| | | | | | | | | | | SEQ |
|---|---|---|---|---|---|---|---|---|---|---|
| Peptide | | | | | | | | | | ID |
| sequence | 1a | 1b | 2 | 6 | 37 | 62 | London | yellow | green | NO |
| MSDNGPQSNQRSAPR | 0.2 | 0.1 | 0.4 | 0.3 | 0.4 | 0.4 | 0.4 | 0.1 | 0.4 | 1123 |
| SDNGPQSNQRSAPRI | 0.3 | 0.0 | 0.3 | 0.3 | 0.5 | 0.4 | 0.4 | 0.1 | 0.2 | 1124 |
| DNGPQSNQRSAPRIT | 0.2 | 0.1 | 0.1 | 0.2 | 0.5 | 0.3 | 0.2 | 0.1 | 0.2 | 1125 |
| NGPQSNQRSAPRITF | 0.3 | 0.2 | 0.5 | 0.3 | 0.8 | 0.6 | 0.5 | 0.6 | 0.5 | 592 |
| GPQSNQRSAPRITFG | 0.3 | 0.2 | 0.3 | 0.3 | 0.6 | 0.4 | 0.4 | 0.8 | 0.3 | 593 |
| PQSNQRSAPRITFGG | 0.5 | 0.3 | 0.5 | 0.5 | 0.8 | 0.7 | 0.5 | 0.6 | 0.4 | 594 |
| QSNQRSAPRITFGGP | 0.5 | 0.3 | 0.6 | 0.4 | 0.8 | 0.7 | 0.5 | 0.7 | 0.5 | 595 |
| SNQRSAPRITFGGPT | 0.4 | 0.2 | 0.4 | 0.4 | 0.7 | 0.4 | 0.9 | 0.6 | 0.3 | 596 |
| NQRSAPRITFGGPTD | 0.4 | 0.3 | 0.4 | 0.2 | 0.6 | 0.4 | 0.4 | 0.7 | 0.5 | 597 |
| QRSAPRITFGGPTDS | 0.4 | 0.3 | 0.4 | 0.4 | 0.7 | 0.5 | 0.9 | 0.6 | 0.4 | 598 |
| RSAPRITFGGPTDST | 0.3 | 0.2 | 0.3 | 0.2 | 0.5 | 0.4 | 0.3 | 0.5 | 0.4 | 599 |
| SAPRITFGGPTDSTD | 0.3 | 0.2 | 0.2 | 0.1 | 0.5 | 0.3 | 0.3 | 0.4 | 0.9 | 600 |
| APRITFGGPTDSTDN | 0.4 | 0.2 | 0.3 | 0.2 | 0.6 | 0.6 | 0.2 | 0.4 | 0.6 | 601 |
| PRITFGGPTDSTDNN | 0.3 | 0.2 | 0.3 | 0.2 | 0.7 | 0.5 | 0.3 | 0.4 | 0.8 | 602 |
| RITFGGPTDSTDNNQ | 0.3 | 0.2 | 0.2 | 0.2 | 0.5 | 0.3 | 0.3 | 0.2 | 0.4 | 603 |
| ITFGGPTDSTDNNQN | 0.3 | 0.2 | 0.3 | 0.2 | 0.6 | 0.5 | 0.4 | 0.3 | 0.7 | 604 |
| TFGGPTDSTDNNQNG | 0.3 | 0.2 | 0.3 | 0.2 | 0.4 | 0.3 | 0.3 | 0.1 | 0.5 | 1126 |
| FGGPTDSTDNNQNGG | 0.2 | 0.1 | 0.2 | 0.1 | 0.4 | 0.2 | 0.2 | 0.1 | 0.3 | 1127 |
| GGPTDSTDNNQNGGR | 0.3 | 0.1 | 0.4 | 0.2 | 0.5 | 0.3 | 0.3 | 0.2 | 0.2 | 1128 |
| GPTDSTDNNQNGGRN | 0.4 | 0.4 | 0.6 | 0.3 | 0.9 | 0.6 | 0.5 | 0.4 | 1.4 | 1129 |
| PTDSTDNNQNGGRNG | 0.3 | 0.2 | 0.3 | 0.2 | 0.5 | 0.3 | 0.4 | 0.5 | 0.4 | 1130 |
| TDSTDNNQNGGRNGA | 0.4 | 0.3 | 0.6 | 0.3 | 0.7 | 0.3 | 0.6 | 0.7 | 0.8 | 1131 |
| DSTDNNQNGGRNGAR | 0.4 | 0.3 | 0.5 | 0.3 | 0.7 | 0.4 | 0.5 | 0.5 | 0.8 | 1132 |
| STDNNQNGGRNGARP | 0.3 | 0.2 | 0.3 | 0.2 | 0.5 | 0.2 | 0.3 | 0.3 | 0.4 | 1133 |
| TDNNQNGGRNGARPK | 0.2 | 0.1 | 0.2 | 0.2 | 0.4 | 0.2 | 0.3 | 0.3 | 0.1 | 1134 |
| DNNQNGGRNGARPKQ | 0.4 | 0.2 | 0.4 | 0.3 | 0.6 | 0.4 | 0.4 | 0.4 | 0.4 | 1135 |
| NNQNGGRNGARPKQR | 0.2 | 0.1 | 0.2 | 0.2 | 0.4 | 0.3 | 0.3 | 0.3 | 0.1 | 1136 |
| NQNGGRNGARPKQRR | 0.2 | 0.1 | 0.2 | 0.2 | 0.4 | 0.3 | 0.3 | 0.3 | 0.2 | 1137 |
| QNGGRNGARPKQRRP | 0.3 | 0.1 | 0.3 | 0.2 | 0.6 | 0.4 | 0.3 | 0.5 | 0.6 | 1138 |
| NGGRNGARPKQRRPQ | 0.3 | 0.2 | 0.3 | 0.3 | 0.6 | 0.5 | 0.3 | 0.5 | 0.4 | 1139 |
| GGRNGARPKQRRPQG | 0.2 | 0.1 | 0.2 | 0.2 | 0.4 | 0.3 | 0.2 | 0.2 | 0.2 | 1140 |
| GRNGARPKQRRPQGL | 0.2 | 0.1 | 0.2 | 0.2 | 0.4 | 0.3 | 0.3 | 0.3 | 0.2 | 1141 |
| RNGARPKQRRPQGLP | 0.2 | 0.1 | 0.3 | 0.2 | 0.5 | 0.4 | 0.4 | 0.2 | 0.3 | 1142 |
| NGARPKQRRPQGLPN | 0.3 | 0.2 | 0.3 | 0.4 | 0.6 | 0.5 | 0.5 | 0.4 | 0.4 | 1143 |
| GARPKQRRPQGLPNN | 0.3 | 0.1 | 0.4 | 0.3 | 0.4 | 0.5 | 0.4 | 0.2 | 0.4 | 1144 |
| ARPKQRRPQGLPNNT | 0.2 | 0.1 | 0.4 | 0.2 | 0.5 | 0.5 | 0.3 | 0.2 | 0.2 | 1145 |
| RPKQRRPQGLPNNTA | 0.3 | 0.1 | 0.3 | 0.3 | 0.5 | 0.5 | 0.4 | 0.3 | 0.2 | 1146 |
| PKQRRPQGLPNNTAS | 0.5 | 0.5 | 0.8 | 0.4 | 0.8 | 0.7 | 0.6 | 0.9 | 3.1 | 1147 |
| KQRRPQGLPNNTASW | 0.4 | 0.2 | 0.4 | 0.4 | 0.6 | 0.5 | 0.6 | 0.7 | 0.3 | 1148 |
| QRRPQGLPNNTASWF | 0.5 | 0.3 | 0.5 | 0.4 | 0.7 | 0.6 | 0.5 | 0.6 | 0.7 | 1149 |
| RRPQGLPNNTASWFT | 0.4 | 0.3 | 0.5 | 0.3 | 0.6 | 0.5 | 0.4 | 0.5 | 0.6 | 1150 |
| RPQGLPNNTASWFTA | 0.5 | 0.3 | 0.5 | 0.4 | 0.9 | 0.8 | 0.5 | 0.7 | 0.5 | 1151 |
| PQGLPNNTASWFTAL | 0.5 | 0.3 | 0.6 | 0.3 | 1.0 | 0.9 | 0.6 | 0.9 | 0.4 | 1152 |
| QGLPNNTASWFTALT | 0.4 | 0.3 | 0.5 | 0.3 | 0.7 | 0.7 | 0.5 | 0.7 | 0.6 | 1153 |
| GLPNNTASWFTALTQ | 0.4 | 0.2 | 0.4 | 0.3 | 0.7 | 0.7 | 0.4 | 0.5 | 0.6 | 1154 |
| LPNNTASWFTALTQH | 0.4 | 0.3 | 0.4 | 0.3 | 0.8 | 0.7 | 0.3 | 0.7 | 0.7 | 1155 |
| PNNTASWFTALTQHG | 0.4 | 0.2 | 0.3 | 0.2 | 0.6 | 0.5 | 0.3 | 0.6 | 0.7 | 1156 |
| NNTASWFTALTQHGK | 0.2 | 0.1 | 0.2 | 0.1 | 0.4 | 0.2 | 0.2 | 0.1 | 0.1 | 1157 |
| NTASWFTALTQHGKE | 0.2 | 0.2 | 0.2 | 0.1 | 0.4 | 0.3 | 0.3 | 0.3 | 0.3 | 1158 |
| TASWFTALTQHGKEE | 0.2 | 0.1 | 0.1 | 0.1 | 0.4 | 0.2 | 0.3 | 0.2 | 0.1 | 1159 |
| ASWFTALTQHGKEEL | 0.2 | 0.1 | 0.2 | 0.1 | 0.4 | 0.3 | 0.3 | 0.1 | 0.2 | 1160 |
| SWFTALTQHGKEELR | 0.2 | 0.1 | 0.2 | 0.1 | 0.3 | 0.2 | 0.2 | 0.0 | 0.1 | 1161 |
| WFTALTQHGKEELRF | 0.4 | 0.2 | 0.4 | 0.2 | 0.8 | 0.6 | 0.4 | 0.4 | 0.5 | 1162 |
| FTALTQHGKEELRFP | 0.2 | 0.1 | 0.3 | 0.1 | 0.4 | 0.3 | 0.2 | 0.1 | 0.0 | 1163 |
| TALTQHGKEELRFPR | 0.4 | 0.3 | 0.6 | 0.5 | 0.8 | 0.6 | 0.6 | 0.7 | 0.7 | 1164 |
| ALTQHGKEELRFPRG | 0.2 | 0.1 | 0.2 | 0.1 | 0.5 | 0.2 | 0.3 | 0.3 | 0.1 | 1165 |
| LTQHGKEELRFPRGQ | 0.4 | 0.2 | 0.4 | 0.3 | 0.6 | 0.4 | 0.4 | 0.4 | 0.3 | 1166 |
| TQHGKEELRFPRGQG | 0.3 | 0.2 | 0.3 | 0.2 | 0.5 | 0.3 | 0.4 | 0.4 | 0.2 | 1167 |
| QHGKEELRFPRGQGV | 0.4 | 0.3 | 0.5 | 0.4 | 0.6 | 0.4 | 0.6 | 0.7 | 0.4 | 1168 |
| HGKEELRFPRGQGVP | 0.3 | 0.2 | 0.4 | 0.2 | 0.5 | 0.3 | 0.4 | 0.5 | 0.2 | 1169 |
| GKEELRFPRGQGVPI | 0.5 | 0.4 | 0.8 | 0.3 | 0.9 | 1.1 | 0.6 | 0.7 | 0.9 | 1170 |
| KEELRFPRGQGVPIN | 0.4 | 0.3 | 0.5 | 0.4 | 0.7 | 0.5 | 0.7 | 0.6 | 0.4 | 1171 |
| EELRFPRGQGVPINT | 0.6 | 0.4 | 0.8 | 0.5 | 1.0 | 1.2 | 0.7 | 0.8 | 0.9 | 1172 |
| ELRFPRGQGVPINTN | 0.4 | 0.3 | 0.5 | 0.3 | 0.7 | 0.6 | 0.5 | 0.4 | 0.7 | 1173 |
| LRFPRGQGVPINTNS | 0.3 | 0.2 | 0.4 | 0.2 | 0.6 | 0.4 | 0.4 | 0.4 | 0.6 | 1174 |
| RFPRGQGVPINTNSG | 0.3 | 0.2 | 0.4 | 0.2 | 0.5 | 0.4 | 0.3 | 0.4 | 0.5 | 1175 |
| FPRGQGVPINTNSGP | 0.3 | 0.2 | 0.4 | 0.2 | 0.5 | 0.4 | 0.4 | 0.4 | 0.4 | 1176 |
| PRGQGVPINTNSGPD | 0.2 | 0.1 | 0.2 | 0.1 | 0.3 | 0.2 | 0.2 | 0.1 | 0.1 | 1177 |
| RGQGVPINTNSGPDD | 0.2 | 0.1 | 0.2 | 0.1 | 0.3 | 0.2 | 0.2 | 0.0 | 0.2 | 1178 |
| GQGVPINTNSGPDDQ | 0.2 | 0.1 | 0.3 | 0.1 | 0.4 | 0.1 | 0.3 | 0.1 | 0.1 | 1179 |
| QGVPINTNSGPDDQI | 0.5 | 0.4 | 0.6 | 0.4 | 0.6 | 1.0 | 0.6 | 1.7 | 1.3 | 1180 |
| GVPINTNSGPDDQIG | 0.3 | 0.2 | 0.2 | 0.1 | 0.5 | 0.3 | 0.3 | 0.2 | 0.3 | 1181 |
| VPINTNSGPDDQIGY | 0.5 | 0.4 | 0.4 | 0.2 | 0.9 | 0.7 | 0.5 | 0.7 | 1.1 | 1182 |
| PINTNSGPDDQIGYY | 0.4 | 0.4 | 0.4 | 0.2 | 0.7 | 0.5 | 0.4 | 0.5 | 0.8 | 1183 |
| INTNSGPDDQIGYYR | 0.4 | 0.3 | 0.4 | 0.2 | 0.7 | 0.5 | 0.4 | 0.5 | 0.5 | 1184 |
| NTNSGPDDQIGYYRR | 0.5 | 0.4 | 0.5 | 0.3 | 0.9 | 0.8 | 0.5 | 0.6 | 0.6 | 1185 |
| TNSGPDDQIGYYRRA | 0.5 | 0.4 | 0.5 | 0.3 | 0.9 | 0.8 | 0.6 | 0.7 | 0.6 | 1186 |
| NSGPDDQIGYYRRAT | 0.5 | 0.3 | 0.5 | 0.3 | 1.0 | 0.7 | 0.5 | 0.6 | 0.5 | 1187 |
| SGPDDQIGYYRRATR | 0.4 | 0.3 | 0.5 | 0.4 | 0.8 | 0.7 | 0.5 | 0.7 | 0.5 | 545 |
| GPDDQIGYYRRATRR | 0.4 | 0.3 | 0.5 | 0.4 | 0.8 | 0.8 | 0.5 | 0.9 | 0.6 | 546 |
| PDDQIGYYRRATRRV | 0.4 | 0.3 | 0.6 | 0.5 | 0.9 | 0.8 | 0.5 | 0.7 | 0.8 | 547 |
| DDQIGYYRRATRRVR | 0.4 | 0.2 | 0.5 | 0.4 | 0.9 | 0.7 | 0.5 | 0.5 | 0.4 | 548 |
| DQIGYYRRATRRVRG | 0.3 | 0.2 | 0.5 | 0.3 | 0.9 | 0.8 | 0.5 | 0.7 | 0.6 | 549 |
| QIGYYRRATRRVRGG | 0.3 | 0.2 | 0.4 | 0.3 | 0.7 | 0.6 | 0.4 | 0.6 | 0.4 | 550 |
| IGYYRRATRRVRGGD | 0.3 | 0.2 | 0.5 | 0.3 | 0.7 | 0.6 | 0.4 | 0.5 | 0.3 | 551 |
| GYYRRATRRVRGGDG | 0.3 | 0.1 | 0.4 | 0.2 | 0.5 | 0.4 | 0.3 | 0.2 | 0.2 | 552 |
| YYRRATRRVRGGDGK | 0.2 | 0.1 | 0.2 | 0.1 | 0.4 | 0.2 | 0.2 | 0.1 | 0.0 | 1188 |
| YRRATRRVRGGDGKM | 0.2 | 0.1 | 0.2 | 0.1 | 0.4 | 0.3 | 0.3 | 0.1 | 0.2 | 1189 |
| RRATRRVRGGDGKMK | 0.2 | 0.1 | 0.1 | 0.1 | 0.4 | 0.3 | 0.2 | 0.2 | 0.0 | 1190 |
| RATRRVRGGDGKMKE | 0.2 | 0.1 | 0.2 | 0.1 | 0.5 | 0.2 | 0.2 | 0.3 | 0.0 | 1191 |
| ATRRVRGGDGKMKEL | 0.2 | 0.1 | 0.2 | 0.1 | 0.4 | 0.3 | 0.3 | 0.4 | 0.1 | 1192 |
| TRRVRGGDGKMKELS | 0.2 | 0.1 | 0.2 | 0.2 | 0.4 | 0.3 | 0.3 | 0.2 | 0.0 | 1193 |
| RRVRGGDGKMKELSP | 0.3 | 0.1 | 0.3 | 0.2 | 0.4 | 0.3 | 0.4 | 0.3 | 0.1 | 1194 |
| RVRGGDGKMKELSPR | 0.3 | 0.2 | 0.4 | 0.4 | 0.6 | 0.5 | 0.4 | 0.6 | 0.1 | 1195 |
| VRGGDGKMKELSPRW | 0.3 | 0.2 | 0.5 | 0.2 | 0.6 | 0.6 | 0.5 | 0.3 | 0.1 | 1196 |
| RGGDGKMKELSPRWY | 0.3 | 0.2 | 0.4 | 0.3 | 0.5 | 0.4 | 0.4 | 0.4 | 0.3 | 1197 |
| GGDGKMKELSPRWYF | 0.3 | 0.2 | 0.6 | 0.3 | 0.6 | 0.6 | 0.5 | 0.6 | 0.5 | 1198 |
| GDGKMKELSPRWYFY | 0.4 | 0.3 | 0.6 | 0.3 | 0.7 | 0.6 | 0.5 | 0.7 | 0.5 | 1199 |
| DGKMKELSPRWYFYY | 0.4 | 0.3 | 0.5 | 0.4 | 0.8 | 0.7 | 0.5 | 0.5 | 0.4 | 1200 |
| GKMKELSPRWYFYYL | 0.3 | 0.2 | 0.5 | 0.4 | 0.8 | 0.8 | 0.7 | 0.6 | 0.4 | 1201 |
| KMKELSPRWYFYYLG | 0.3 | 0.2 | 0.4 | 0.3 | 0.7 | 0.7 | 0.4 | 0.6 | 0.4 | 1202 |
| MKELSPRWYFYYLGT | 0.4 | 0.2 | 0.4 | 0.2 | 0.7 | 0.6 | 0.4 | 0.4 | 0.4 | 1203 |
| KELSPRWYFYYLGTG | 0.4 | 0.2 | 0.4 | 0.3 | 0.7 | 0.5 | 0.4 | 0.3 | 0.4 | 1204 |
| ELSPRWYFYYLGTGP | 0.3 | 0.2 | 0.4 | 0.2 | 0.8 | 0.7 | 0.4 | 0.5 | 0.3 | 1205 |
| LSPRWYFYYLGTGPE | 0.3 | 0.3 | 0.5 | 0.2 | 0.8 | 0.8 | 0.3 | 0.5 | 0.6 | 1206 |
| SPRWYFYYLGTGPEA | 0.6 | 0.3 | 0.5 | 0.4 | 0.9 | 1.1 | 0.7 | 0.7 | 0.6 | 1207 |
| PRWYFYYLGTGPEAS | 0.4 | 0.3 | 0.5 | 0.3 | 1.0 | 0.8 | 0.5 | 0.8 | 0.9 | 1208 |
| RWYFYYLGTGPEASL | 0.4 | 0.4 | 0.5 | 0.3 | 1.1 | 0.9 | 0.5 | 0.7 | 0.8 | 1209 |
| WYFYYLGTGPEASLP | 0.4 | 0.3 | 0.4 | 0.3 | 0.7 | 0.5 | 0.4 | 0.5 | 0.4 | 1210 |
| YFYYLGTGPEASLPY | 0.4 | 0.3 | 0.5 | 0.3 | 0.9 | 0.7 | 0.4 | 0.6 | 0.4 | 1211 |
| FYYLGTGPEASLPYG | 0.4 | 0.3 | 0.4 | 0.2 | 0.7 | 0.6 | 0.5 | 0.7 | 0.4 | 1212 |
| YYLGTGPEASLPYGA | 0.4 | 0.4 | 0.4 | 0.3 | 0.9 | 0.7 | 0.5 | 0.7 | 0.5 | 1213 |
| YLGTGPEASLPYGAN | 0.4 | 0.3 | 0.4 | 0.2 | 0.8 | 0.7 | 0.5 | 0.6 | 0.6 | 1214 |
| LGTGPEASLPYGANK | 0.3 | 0.2 | 0.5 | 0.2 | 0.5 | 0.4 | 0.4 | 0.5 | 0.4 | 1215 |
| GTGPEASLPYGANKE | 0.2 | 0.2 | 0.2 | 0.1 | 0.4 | 0.3 | 0.3 | 0.2 | 0.2 | 1216 |
| TGPEASLPYGANKEG | 0.3 | 0.2 | 0.5 | 0.4 | 0.6 | 0.5 | 0.6 | 0.4 | 0.7 | 1217 |
| GPEASLPYGANKEGI | 0.3 | 0.2 | 0.4 | 0.2 | 0.6 | 0.5 | 0.4 | 0.3 | 0.5 | 1218 |
| PEASLPYGANKEGIV | 0.3 | 0.2 | 0.4 | 0.2 | 0.4 | 0.3 | 0.3 | 0.2 | 0.3 | 1219 |
| EASLPYGANKEGIVW | 0.3 | 0.2 | 0.4 | 0.2 | 0.7 | 0.6 | 0.5 | 0.2 | 0.5 | 1220 |
| ASLPYGANKEGIVWV | 0.4 | 0.3 | 0.7 | 0.3 | 1.0 | 1.0 | 0.6 | 0.3 | 0.8 | 1221 |
| SLPYGANKEGIVWVA | 0.3 | 0.2 | 0.5 | 0.3 | 0.7 | 0.6 | 0.4 | 0.4 | 0.3 | 1222 |
| LPYGANKEGIVWVAT | 0.5 | 0.3 | 0.6 | 0.4 | 0.9 | 0.8 | 0.5 | 0.4 | 0.4 | 1223 |
| PYGANKEGIVWVATE | 0.4 | 0.3 | 0.5 | 0.3 | 0.9 | 0.9 | 0.5 | 0.7 | 0.8 | 1224 |
| YGANKEGIVWVATEG | 0.4 | 0.3 | 0.5 | 0.3 | 0.8 | 0.7 | 0.4 | 0.7 | 0.4 | 1225 |
| GANKEGIVWVATEGA | 0.2 | 0.1 | 0.2 | 0.1 | 0.5 | 0.3 | 0.3 | 0.4 | 0.2 | 1226 |
| ANKEGTVWVATEGAL | 0.4 | 0.3 | 0.3 | 0.2 | 0.6 | 0.5 | 0.3 | 0.6 | 0.4 | 1227 |
| NKEGIVWVATEGALN | 0.3 | 0.2 | 0.4 | 0.2 | 0.6 | 0.4 | 0.3 | 0.4 | 0.3 | 1228 |
| KEGIVWVATEGALNT | 0.3 | 0.2 | 0.4 | 0.2 | 0.6 | 0.5 | 0.4 | 0.5 | 0.3 | 1229 |
| EGIVWVATEGALNTP | 0.3 | 0.2 | 0.4 | 0.2 | 0.7 | 0.6 | 0.3 | 0.6 | 0.4 | 1230 |
| GIVWVATEGALNTPK | 0.3 | 0.1 | 0.4 | 0.2 | 0.5 | 0.4 | 0.4 | 0.3 | 0.3 | 1231 |
| IVWVATEGALNTPKD | 0.3 | 0.2 | 0.3 | 0.1 | 0.5 | 0.3 | 0.3 | 0.2 | 0.9 | 1232 |
| VWVATEGALNTPKDH | 0.3 | 0.3 | 0.6 | 0.4 | 0.6 | 0.6 | 1.2 | 0.6 | 0.7 | 1233 |
| WVATEGALNTPKDHI | 0.3 | 0.2 | 0.3 | 0.2 | 0.5 | 0.4 | 0.4 | 0.2 | 0.4 | 1234 |
| VATEGALNTPKDHIG | 0.3 | 0.2 | 0.3 | 0.2 | 0.4 | 0.3 | 0.3 | 0.2 | 0.2 | 1235 |
| ATEGALNTPKDHIGT | 0.3 | 0.2 | 0.4 | 0.2 | 0.4 | 0.3 | 0.3 | 0.2 | 0.3 | 1236 |
| TEGALNTPKDHIGTR | 0.3 | 0.2 | 0.5 | 0.4 | 0.5 | 0.6 | 0.4 | 0.4 | 0.6 | 1237 |
| EGALNTPKDHIGTRN | 0.3 | 0.2 | 0.5 | 0.3 | 0.6 | 0.3 | 0.5 | 0.2 | 0.4 | 1238 |
| GALNTPKDHIGTRNP | 0.2 | 0.1 | 0.5 | 0.2 | 0.5 | 0.3 | 0.4 | 0.2 | 0.2 | 1239 |
| ALNTPKDHIGTRNPN | 0.4 | 0.3 | 0.5 | 0.3 | 0.5 | 0.5 | 0.3 | 0.0 | 0.5 | 1240 |
| LNTPKDHIGTRNPNN | 0.4 | 0.3 | 0.4 | 0.3 | 0.6 | 0.5 | 0.5 | 0.4 | 0.4 | 1241 |
| NTPKDHIGTRNPNNN | 0.4 | 0.3 | 0.4 | 0.3 | 0.6 | 0.3 | 0.4 | 0.4 | 0.4 | 1242 |
| TPKDHIGTRNPNNNA | 0.3 | 0.3 | 0.3 | 0.2 | 0.5 | 0.3 | 0.3 | 0.3 | 0.3 | 1243 |
| PKDHIGTRNPNNNAA | 0.4 | 0.3 | 0.3 | 0.3 | 0.5 | 0.3 | 0.3 | 0.2 | 0.2 | 1244 |
| KDHIGTRNPNNNAAT | 0.4 | 0.3 | 0.5 | 0.4 | 0.5 | 0.4 | 0.4 | 0.3 | 0.1 | 1245 |
| DHIGTRNPNNNAATV | 0.5 | 0.4 | 0.8 | 0.5 | 1.1 | 0.8 | 0.7 | 0.6 | 0.5 | 1246 |
| HIGTRNPNNNAATVL | 0.6 | 0.5 | 0.9 | 0.6 | 1.2 | 1.3 | 0.7 | 0.8 | 0.9 | 1247 |
| IGTRNPNNNAATVLQ | 0.4 | 0.3 | 0.7 | 0.4 | 0.9 | 0.8 | 0.6 | 0.5 | 0.7 | 1248 |
| GTRNPNNNAATVLQL | 0.4 | 0.3 | 0.7 | 0.4 | 0.9 | 0.9 | 0.5 | 0.5 | 0.6 | 1249 |
| TRNPNNNAATVLQLP | 0.4 | 0.3 | 0.7 | 0.3 | 0.6 | 0.5 | 0.5 | 0.5 | 0.8 | 1250 |
| RNPNNNAATVLQLPQ | 0.4 | 0.4 | 0.7 | 0.3 | 0.8 | 0.9 | 0.6 | 0.4 | 0.6 | 1251 |
| NPNNNAATVLQLPQG | 0.4 | 0.4 | 0.5 | 0.3 | 0.9 | 0.9 | 0.5 | 0.5 | 0.7 | 1252 |
| PNNNAATVLQLPQGT | 0.5 | 0.4 | 0.8 | 0.3 | 1.0 | 0.9 | 0.7 | 0.5 | 0.7 | 1253 |
| NNNAATVLQLPQGTT | 0.4 | 0.3 | 0.5 | 0.2 | 0.6 | 0.5 | 0.5 | 0.4 | 0.5 | 1254 |
| 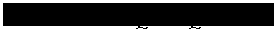 | 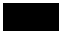 | 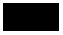 | 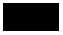 | 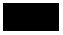 | 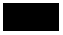 | 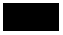 | 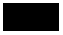 | 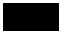 | 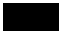 | 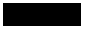 |
| 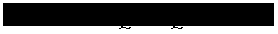 | 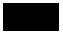 | 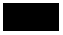 | 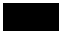 | 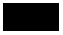 | 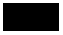 | 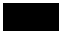 | 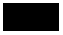 | 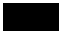 | 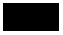 | 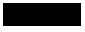 |
| 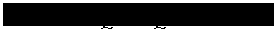 | 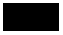 | 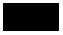 | 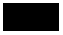 | 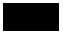 | 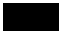 | 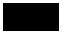 | 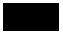 | 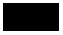 | 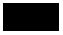 | 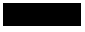 |
| 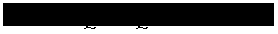 | 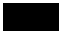 | 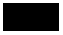 | 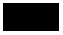 | 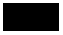 | 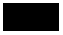 | 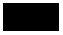 | 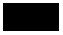 | 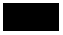 | 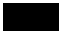 | 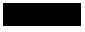 |
| 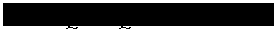 | 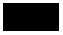 | 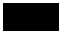 | 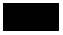 | 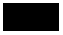 | 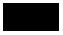 | 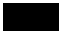 | 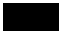 | 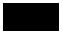 | 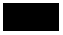 | 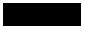 |
| 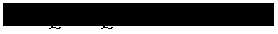 | 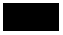 | 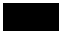 | 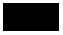 | 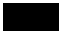 | 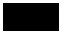 | 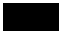 | 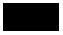 | 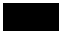 | 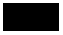 | 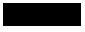 |
| 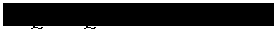 | 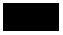 | 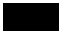 | 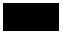 | 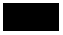 | 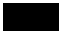 | 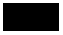 | 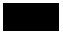 | 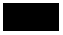 | 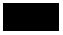 | 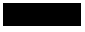 |
| 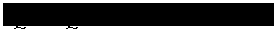 | 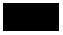 | 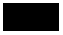 | 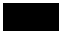 | 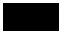 | 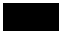 | 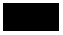 | 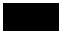 | 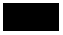 | 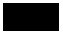 | 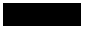 |
| 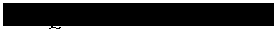 | 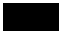 | 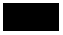 | 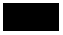 | 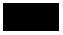 | 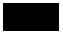 | 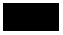 | 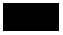 | 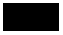 | 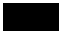 | 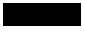 |
| 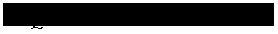 | 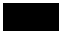 | 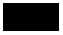 | 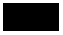 | 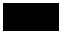 | 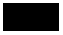 | 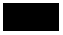 | 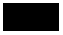 | 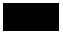 | 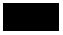 | 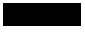 |
| 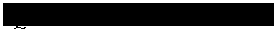 | 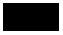 | 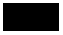 | 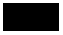 | 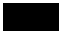 | 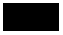 | 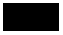 | 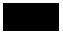 | 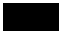 | 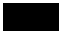 | 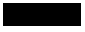 |
| 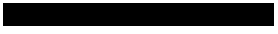 | 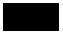 | 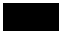 | 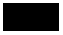 | 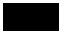 | 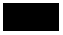 | 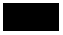 | 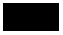 | 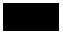 | 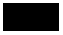 | 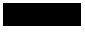 |
| 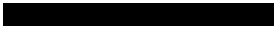 | 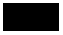 | 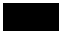 | 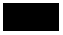 | 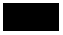 | 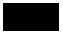 | 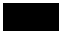 | 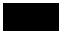 | 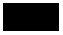 | 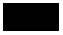 | 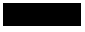 |
| 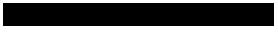 | 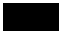 | 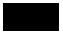 | 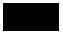 | 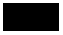 | 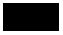 | 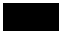 | 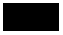 | 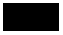 | 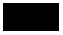 | 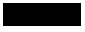 |
| LPKGFYAEGSRGGSQ | 0.3 | 0.2 | 0.3 | 0.2 | 0.5 | 0.4 | 0.4 | 0.2 | 0.3 | 1255 |
| PKGFYAEGSRGGSQA | 0.3 | 0.3 | 0.3 | 0.2 | 0.4 | 0.5 | 0.3 | 0.2 | 0.4 | 1256 |
| KGFYAEGSRGGSQAS | 0.3 | 0.2 | 0.3 | 0.2 | 0.4 | 0.3 | 0.3 | 0.1 | 0.1 | 1257 |
| GFYAEGSRGGSQASS | 0.3 | 0.2 | 0.4 | 0.3 | 0.3 | 0.2 | 0.3 | 0.1 | 0.3 | 1258 |
| FYAEGSRGGSQASSR | 0.3 | 0.2 | 0.4 | 0.3 | 0.6 | 0.6 | 0.4 | 0.3 | 0.4 | 1259 |
| YAEGSRGGSQASSRS | 0.3 | 0.2 | 0.3 | 0.2 | 0.4 | 0.2 | 0.3 | 0.1 | 0.3 | 1260 |
| AEGSRGGSQASSRSS | 0.4 | 0.2 | 0.4 | 0.5 | 0.5 | 0.5 | 0.4 | 0.4 | 0.4 | 1261 |
| EGSRGGSQASSRSSS | 0.4 | 0.3 | 0.4 | 0.6 | 0.6 | 0.4 | 0.4 | 0.7 | 0.7 | 1262 |
| GSRGGSQASSRSSSR | 0.6 | 0.8 | 0.7 | 0.5 | 0.9 | 0.7 | 0.7 | 1.6 | 1.2 | 1263 |
| SRGGSQASSRSSSRS | 0.4 | 0.3 | 0.3 | 0.3 | 0.5 | 0.3 | 0.3 | 0.2 | 0.2 | 1264 |
| RGGSQASSRSSSRSR | 0.2 | 0.2 | 0.4 | 0.3 | 0.2 | 0.1 | 0.1 | 0.3 | 0.2 | 1265 |
| GGSQASSRSSSRSRG | 0.3 | 0.2 | 0.4 | 0.4 | 0.5 | 0.4 | 0.4 | 0.3 | 0.0 | 1266 |
| GSQASSRSSSRSRGN | 0.4 | 0.2 | 0.4 | 0.3 | 0.7 | 0.6 | 0.5 | 0.5 | 0.1 | 1267 |
| SQASSRSSSRSRGNS | 0.3 | 0.2 | 0.3 | 0.2 | 0.5 | 0.4 | 0.4 | 0.3 | 0.0 | 1268 |
| QASSRSSSRSRGNSR | 0.3 | 0.2 | 0.4 | 0.3 | 0.5 | 0.4 | 0.4 | 0.4 | 0.1 | 1269 |
| ASSRSSSRSRGNSRN | 0.3 | 0.2 | 0.4 | 0.3 | 0.6 | 0.5 | 0.4 | 0.4 | 0.2 | 1270 |
| SSRSSSRSRGNSRNS | 0.3 | 0.3 | 0.4 | 0.3 | 0.5 | 0.6 | 0.5 | 0.4 | 0.3 | 1271 |
| SRSSSRSRGNSRNST | 0.3 | 0.2 | 0.3 | 0.3 | 0.5 | 0.4 | 0.4 | 0.4 | 0.2 | 1272 |
| RSSSRSRGNSRNSTP | 0.2 | 0.2 | 0.2 | 0.1 | 0.5 | 0.3 | 0.3 | 0.3 | 0.2 | 1273 |
| SSSRSRGNSRNSTPG | 0.2 | 0.1 | 0.3 | 0.2 | 0.4 | 0.3 | 0.3 | 0.1 | 0.1 | 1274 |
| SSRSRGNSRNSTPGS | 0.3 | 0.1 | 0.3 | 0.1 | 0.4 | 0.3 | 0.3 | 0.1 | 0.0 | 1275 |
| SRSRGNSRNSTPGSS | 0.5 | 0.4 | 0.3 | 0.1 | 0.3 | 0.3 | 0.3 | 0.0 | 0.0 | 1276 |
| RSRGNSRNSTPGSSR | 0.3 | 0.1 | 0.2 | 0.2 | 0.2 | 0.1 | 0.2 | 0.1 | 0.1 | 1277 |
| SRGNSRNSTPGSSRG | 0.6 | 0.4 | 0.2 | 0.2 | 0.4 | 0.3 | 0.3 | 0.2 | 0.1 | 1278 |
| RGNSRNSTPGSSRGN | 0.8 | 0.6 | 0.4 | 0.4 | 0.6 | 0.5 | 0.4 | 0.5 | 0.3 | 1279 |
| GNSRNSTPGSSRGNS | 0.6 | 0.5 | 0.4 | 0.4 | 0.5 | 0.5 | 0.3 | 0.5 | 0.1 | 1280 |
| NSRNSTPGSSRGNSP | 0.6 | 0.5 | 0.5 | 0.4 | 0.6 | 0.5 | 0.4 | 0.3 | 0.3 | 1281 |
| SRNSTPGSSRGNSPA | 0.6 | 0.6 | 0.4 | 0.4 | 0.5 | 0.5 | 0.4 | 0.4 | 0.3 | 1282 |
| RNSTPGSSRGNSPAR | 0.4 | 0.3 | 0.4 | 0.3 | 0.5 | 0.5 | 0.5 | 0.5 | 0.1 | 553 |
| NSTPGSSRGNSPARM | 0.8 | 0.7 | 0.6 | 0.4 | 0.6 | 0.6 | 0.5 | 0.4 | 0.4 | 554 |
| STPGSSRGNSPARMA | 0.3 | 0.2 | 0.4 | 0.4 | 0.6 | 0.5 | 0.5 | 0.5 | 0.2 | 555 |
| TPGSSRGNSPARMAS | 0.4 | 0.2 | 0.5 | 0.4 | 0.6 | 0.6 | 0.5 | 0.5 | 0.4 | 556 |
| PGSSRGNSPARMASG | 0.4 | 0.2 | 0.5 | 0.4 | 0.6 | 0.6 | 0.5 | 0.4 | 0.5 | 557 |
| GSSRGNSPARMASGG | 0.4 | 0.3 | 0.7 | 0.4 | 0.6 | 0.6 | 0.9 | 0.5 | 0.7 | 558 |
| SSRGNSPARMASGGG | 0.4 | 0.2 | 0.5 | 0.4 | 0.4 | 0.5 | 0.8 | 0.5 | 0.6 | 1283 |
| SRGNSPARMASGGGE | 0.2 | 0.1 | 0.2 | 0.1 | 0.4 | 0.3 | 0.3 | 0.1 | 0.2 | 1284 |
| RGNSPARMASGGGET | 0.4 | 0.3 | 0.4 | 0.3 | 0.4 | 0.4 | 0.8 | 0.1 | 0.1 | 1285 |
| GNSPARMASGGGETA | 0.2 | 0.1 | 0.3 | 0.1 | 0.2 | 0.2 | 0.3 | 0.0 | 0.3 | 1286 |
| 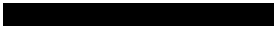 | 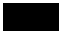 | 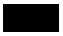 | 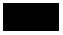 | 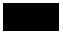 | 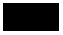 | 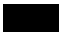 | 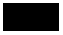 | 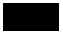 | 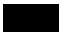 | 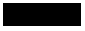 |
| 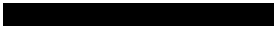 | 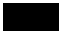 | 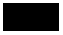 | 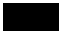 | 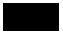 | 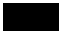 | 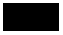 | 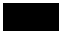 | 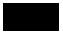 | 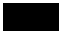 | 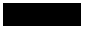 |
| 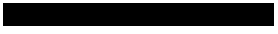 | 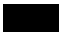 | 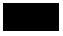 | 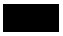 | 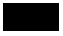 | 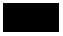 | 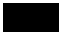 | 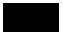 | 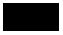 | 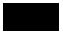 | 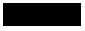 |
| 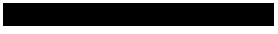 | 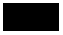 | 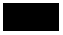 | 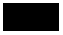 | 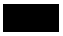 | 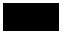 | 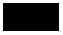 | 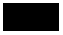 | 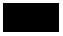 | 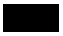 | 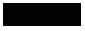 |
| 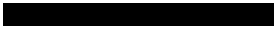 | 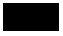 | 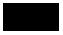 | 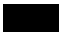 | 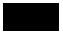 | 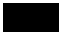 | 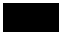 | 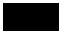 | 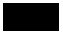 | 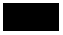 | 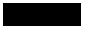 |
| 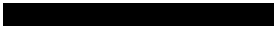 | 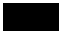 | 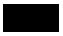 | 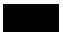 | 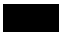 | 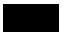 | 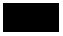 | 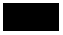 | 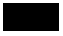 | 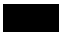 | 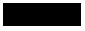 |
| 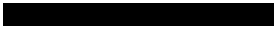 | 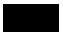 | 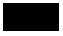 | 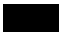 | 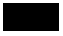 | 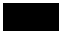 | 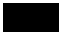 | 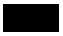 | 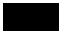 | 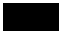 | 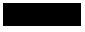 |
| SGGGETALALLLLDR | 0.4 | 0.2 | 0.5 | 0.3 | 0.7 | 0.7 | 0.5 | 0.7 | 0.1 | 1287 |
| GGGETALALLLLDRL | 0.4 | 0.3 | 0.5 | 0.2 | 0.8 | 0.7 | 0.4 | 0.7 | 0.3 | 1288 |
| GGETALALLLLDRLN | 0.4 | 0.3 | 0.5 | 0.2 | 0.7 | 0.7 | 0.5 | 0.7 | 0.3 | 1289 |
| GETALALLLLDRLNQ | 0.4 | 0.2 | 0.4 | 0.2 | 0.7 | 0.7 | 0.5 | 0.6 | 0.3 | 1290 |
| ETALALLLLDRLNQL | 0.4 | 0.3 | 0.4 | 0.2 | 0.7 | 0.6 | 0.5 | 0.5 | 0.4 | 1291 |
| TALALLLLDRLNQLE | 0.4 | 0.3 | 0.5 | 0.3 | 0.8 | 0.7 | 0.5 | 0.5 | 0.7 | 1292 |
| ALALLLLDRLNQLES | 0.4 | 0.2 | 0.4 | 0.2 | 0.8 | 0.7 | 0.5 | 0.5 | 0.4 | 1293 |
| LALLLLDRLNQLESK | 0.3 | 0.2 | 0.4 | 0.2 | 0.5 | 0.5 | 0.4 | 0.4 | 0.6 | 1294 |
| ALLLLDRLNQLESKV | 0.4 | 0.3 | 0.5 | 0.3 | 1.0 | 0.9 | 0.5 | 0.5 | 0.7 | 1295 |
| LLLLDRLNQLESKVS | 0.2 | 0.1 | 0.2 | 0.1 | 0.4 | 0.2 | 0.3 | 0.0 | 0.2 | 1296 |
| LLLDRLNQLESKVSG | 0.3 | 0.1 | 0.4 | 0.1 | 0.5 | 0.3 | 0.3 | 0.2 | 0.6 | 1297 |
| LLDRLNQLESKVSGK | 0.2 | 0.1 | 0.2 | 0.1 | 0.4 | 0.4 | 0.3 | 0.1 | 0.0 | 1298 |
| LDRLNQLESKVSGKG | 0.4 | 0.3 | 0.4 | 0.3 | 0.4 | 0.4 | 0.4 | 0.3 | 0.2 | 1299 |
| DRLNQLESKVSGKGQ | 0.4 | 0.3 | 0.6 | 0.4 | 0.6 | 0.4 | 0.4 | 0.8 | 0.4 | 1300 |
| RLNQLESKVSGKGQQ | 0.4 | 0.3 | 0.5 | 0.4 | 0.6 | 0.7 | 0.4 | 0.8 | 0.4 | 1301 |
| LNQLESKVSGKGQQQ | 0.4 | 0.3 | 0.6 | 0.4 | 0.6 | 0.6 | 0.5 | 0.8 | 0.5 | 1302 |
| NQLESKVSGKGQQQQ | 0.4 | 0.2 | 0.5 | 0.3 | 0.6 | 0.5 | 0.5 | 0.6 | 0.3 | 1303 |
| QLESKVSGKGQQQQG | 0.5 | 0.4 | 0.7 | 0.5 | 0.6 | 0.7 | 0.7 | 1.1 | 0.7 | 1304 |
| LESKVSGKGQQQQGQ | 0.4 | 0.3 | 0.7 | 0.5 | 0.7 | 0.6 | 1.8 | 0.7 | 0.4 | 1305 |
| ESKVSGKGQQQQGQT | 0.6 | 0.4 | 0.8 | 0.4 | 0.7 | 0.7 | 0.8 | 0.9 | 0.7 | 1306 |
| SKVSGKGQQQQGQTV | 0.4 | 0.2 | 0.7 | 0.4 | 0.6 | 0.6 | 0.8 | 0.6 | 0.5 | 1307 |
| KVSGKGQQQQGQTVT | 0.3 | 0.2 | 0.5 | 0.3 | 0.4 | 0.4 | 0.6 | 0.3 | 0.5 | 1308 |
| VSGKGQQQQGQTVTK | 0.9 | 0.3 | 0.5 | 0.3 | 0.5 | 0.5 | 0.5 | 0.6 | 0.5 | 1309 |
| SGKGQQQQGQTVTKK | 0.3 | 0.2 | 0.3 | 0.3 | 0.6 | 0.5 | 0.4 | 0.3 | 0.2 | 1310 |
| GKGQQQQGQTVTKKS | 1.4 | 0.6 | 0.7 | 0.4 | 0.5 | 0.6 | 0.4 | 0.5 | 0.6 | 1311 |
| KGQQQQGQTVTKKSA | 0.2 | 0.1 | 0.2 | 0.1 | 0.4 | 0.3 | 0.3 | 0.1 | 0.0 | 1312 |
| GQQQQGQTVTKKSAA | 0.2 | 0.1 | 0.3 | 0.1 | 0.4 | 0.4 | 0.3 | 0.1 | 0.1 | 1313 |
| QQQQGQTVTKKSAAE | 0.3 | 0.1 | 0.3 | 0.1 | 0.3 | 0.2 | 0.2 | 0.1 | 0.0 | 1314 |
| QQQGQTVTKKSAAEA | 0.5 | 0.3 | 0.7 | 0.5 | 0.6 | 0.7 | 0.6 | 0.6 | 0.9 | 1315 |
| QQGQTVTKKSAAEAS | 0.5 | 0.4 | 0.5 | 0.3 | 0.6 | 0.5 | 0.4 | 0.5 | 0.8 | 379 |
| QGQTVTKKSAAEASK | 0.2 | 0.1 | 0.2 | 0.1 | 0.4 | 0.2 | 0.3 | 0.2 | 0.1 | 380 |
| GQTVTKKSAAEASKK | 0.2 | 0.1 | 0.2 | 0.1 | 0.4 | 0.2 | 0.3 | 0.2 | 0.0 | 381 |
| QTVTKKSAAEASKKP | 0.3 | 0.2 | 0.4 | 0.2 | 0.5 | 0.3 | 0.3 | 0.2 | 0.0 | 382 |
| TVTKKSAAEASKKPR | 0.2 | 0.1 | 0.2 | 0.1 | 0.5 | 0.3 | 0.3 | 0.2 | 0.0 | 383 |
| VTKKSAAEASKKPRQ | 0.3 | 0.2 | 0.6 | 0.3 | 0.6 | 0.4 | 0.5 | 0.7 | 0.6 | 384 |
| TKKSAAEASKKPRQK | 0.2 | 0.1 | 0.2 | 0.1 | 0.4 | 0.3 | 0.3 | 0.2 | 0.0 | 385 |
| KKSAAEASKKPRQKR | 0.2 | 0.1 | 0.3 | 0.2 | 0.5 | 0.3 | 0.4 | 0.3 | 0.2 | 386 |
| KSAAEASKKPRQKRT | 0.2 | 0.1 | 0.2 | 0.1 | 0.4 | 0.2 | 0.3 | 0.1 | 0.1 | 387 |
| SAAEASKKPRQKRTA | 0.2 | 0.1 | 0.2 | 0.1 | 0.4 | 0.3 | 0.3 | 0.2 | 0.1 | 388 |
| AAEASKKPRQKRTAT | 0.2 | 0.1 | 0.2 | 0.1 | 0.4 | 0.3 | 0.3 | 0.2 | 0.1 | 389 |
| AEASKKPRQKRTATK | 0.2 | 0.1 | 0.2 | 0.1 | 0.4 | 0.3 | 0.3 | 0.2 | 0.1 | 1316 |
| EASKKPRQKRTATKQ | 0.3 | 0.2 | 0.4 | 0.3 | 0.6 | 0.5 | 0.5 | 0.4 | 0.3 | 1317 |
| ASKKPRQKRTATKQY | 0.3 | 0.1 | 0.3 | 0.2 | 0.5 | 0.5 | 0.4 | 0.2 | 0.1 | 1318 |
| SKKPRQKRTATKQYN | 0.3 | 0.1 | 0.4 | 0.3 | 0.6 | 0.6 | 0.5 | 0.3 | 0.1 | 1319 |
| KKPRQKRTATKQYNV | 0.3 | 0.1 | 0.4 | 0.3 | 0.6 | 0.5 | 0.5 | 0.4 | 0.1 | 1320 |
| 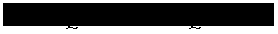 | 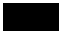 | 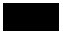 | 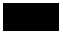 | 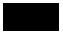 | 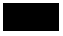 | 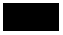 | 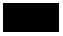 | 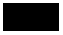 | 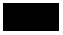 | 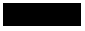 |
| 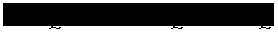 | 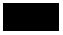 | 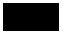 | 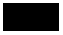 | 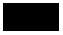 | 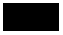 | 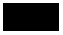 | 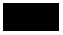 | 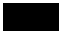 | 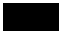 | 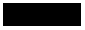 |
| 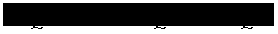 | 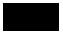 | 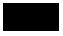 | 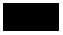 | 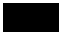 | 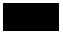 | 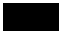 | 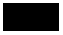 | 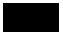 | 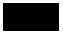 | 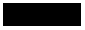 |
| 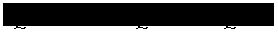 | 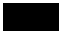 | 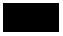 | 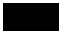 | 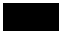 | 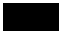 | 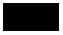 | 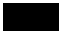 | 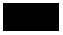 | 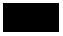 | 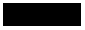 |
| 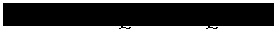 | 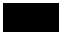 | 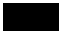 | 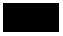 | 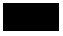 | 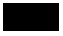 | 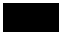 | 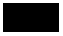 | 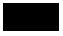 | 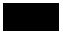 | 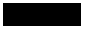 |
| 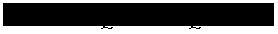 | 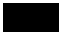 | 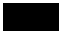 | 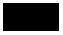 | 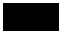 | 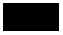 | 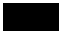 | 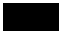 | 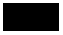 | 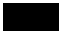 | 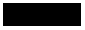 |
| 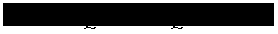 | 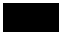 | 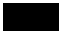 | 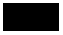 | 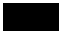 | 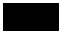 | 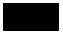 | 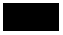 | 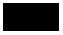 | 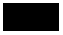 | 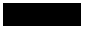 |
| ATKQYNVTQAFGRRG | 0.4 | 0.3 | 0.5 | 0.5 | 0.7 | 0.7 | 1.2 | 0.8 | 0.2 | 565 |
| TKQYNVTQAFGRRGP | 0.4 | 0.2 | 0.5 | 0.3 | 0.7 | 0.7 | 0.5 | 0.6 | 0.4 | 566 |
| KQYNVTQAFGRRGPE | 0.0 | 0.0 | 0.1 | 0.2 | 0.0 | 0.0 | 0.2 | 0.0 | 0.4 | 567 |
| QYNVTQAFGRRGPEQ | 0.4 | 0.2 | 0.4 | 0.4 | 0.7 | 0.6 | 0.5 | 0.5 | 0.4 | 568 |
| YNVTQAFGRRGPEQT | 0.3 | 0.2 | 0.3 | 0.1 | 0.5 | 0.3 | 0.3 | 0.3 | 0.2 | 569 |
| NVTQAFGRRGPEQTQ | 0.3 | 0.2 | 0.3 | 0.2 | 0.5 | 0.4 | 0.4 | 0.3 | 0.3 | 570 |
| VTQAFGRRGPEQTQG | 0.3 | 0.2 | 0.2 | 0.1 | 0.4 | 0.3 | 0.3 | 0.3 | 0.2 | 571 |
| TQAFGRRGPEQTQGN | 0.3 | 0.2 | 0.4 | 0.3 | 0.5 | 0.5 | 0.4 | 0.2 | 0.3 | 572 |
| QAFGRRGPEQTQGNF | 0.3 | 0.2 | 0.3 | 0.2 | 0.3 | 0.3 | 0.3 | 0.2 | 0.2 | 1321 |
| AFGRRGPEQTQGNFG | 0.3 | 0.1 | 0.3 | 0.1 | 0.3 | 0.3 | 0.3 | 0.1 | 0.1 | 1322 |
| FGRRGPEQTQGNFGD | 0.3 | 0.1 | 0.2 | 0.1 | 0.4 | 0.1 | 0.2 | 0.2 | 0.2 | 397 |
| GRRGPEQTQGNFGDQ | 0.4 | 0.3 | 0.4 | 0.2 | 0.5 | 0.6 | 0.5 | 0.5 | 0.4 | 398 |
| RRGPEQTQGNFGDQD | 0.3 | 0.2 | 0.2 | 0.1 | 0.4 | 0.2 | 0.2 | 0.4 | 0.6 | 399 |
| RGPEQTQGNFGDQDL | 0.4 | 0.4 | 0.4 | 0.2 | 0.6 | 0.5 | 0.4 | 0.6 | 0.6 | 400 |
| GPEQTQGNFGDQDLI | 0.4 | 0.4 | 0.4 | 0.2 | 0.6 | 0.4 | 0.3 | 0.6 | 0.9 | 401 |
| PEQTQGNFGDQDLIR | 0.4 | 0.4 | 0.4 | 0.2 | 0.6 | 0.4 | 0.4 | 0.6 | 0.4 | 402 |
| EQTQGNFGDQDLIRQ | 0.3 | 0.1 | 0.4 | 0.3 | 0.2 | 0.3 | 0.3 | 0.3 | 1.3 | 403 |
| QTQGNFGDQDLIRQG | 0.5 | 0.4 | 0.5 | 0.3 | 0.9 | 0.9 | 0.6 | 0.8 | 0.4 | 404 |
| TQGNFGDQDLIRQGT | 0.4 | 0.3 | 0.4 | 0.2 | 0.7 | 0.7 | 0.5 | 0.6 | 0.5 | 1323 |
| QGNFGDQDLIRQGTD | 0.4 | 0.3 | 0.3 | 0.2 | 0.6 | 0.5 | 0.4 | 0.6 | 0.7 | 1324 |
| GNFGDQDLIRQGTDY | 0.5 | 0.3 | 0.5 | 0.3 | 0.8 | 0.8 | 0.5 | 0.6 | 0.7 | 1325 |
| NFGDQDLIRQGTDYK | 0.2 | 0.1 | 0.2 | 0.1 | 0.4 | 0.3 | 0.3 | 0.1 | 0.2 | 1326 |
| FGDQDLIRQGTDYKH | 0.4 | 0.3 | 0.4 | 0.2 | 0.7 | 0.6 | 0.4 | 0.5 | 0.7 | 1327 |
| GDQDLIRQGTDYKHW | 0.4 | 0.2 | 0.4 | 0.3 | 0.6 | 0.5 | 0.4 | 0.3 | 0.4 | 1328 |
| DQDLIRQGTDYKHWP | 0.2 | 0.1 | 0.3 | 0.1 | 0.4 | 0.3 | 0.3 | 0.1 | 0.1 | 1329 |
| QDLIRQGTDYKHWPQ | 0.3 | 0.2 | 0.3 | 0.2 | 0.5 | 0.4 | 0.4 | 0.3 | 0.2 | 1330 |
| DLIRQGTDYKHWPQI | 0.4 | 0.2 | 0.4 | 0.2 | 0.6 | 0.6 | 0.4 | 0.3 | 0.2 | 1331 |
| LIRQGTDYKHWPQIA | 0.4 | 0.5 | 0.5 | 0.3 | 0.6 | 0.5 | 0.3 | 1.1 | 1.0 | 1332 |
| IRQGTDYKHWPQIAQ | 0.4 | 0.2 | 0.4 | 0.2 | 0.6 | 0.6 | 0.4 | 0.4 | 0.3 | 1333 |
| RQGTDYKHWPQIAQF | 0.5 | 0.4 | 0.5 | 0.3 | 0.8 | 0.9 | 0.5 | 0.8 | 0.4 | 1334 |
| QGTDYKHWPQIAQFA | 0.4 | 0.3 | 0.4 | 0.3 | 0.6 | 0.6 | 0.5 | 0.7 | 0.3 | 1335 |
| GTDYKHWPQIAQFAP | 0.5 | 0.3 | 0.5 | 0.3 | 0.7 | 0.7 | 0.5 | 0.7 | 0.5 | 1336 |
| TDYKHWPQIAQFAPS | 0.6 | 0.5 | 0.8 | 0.5 | 0.9 | 1.3 | 0.7 | 0.9 | 0.6 | 1337 |
| DYKHWPQIAQFAPSA | 0.4 | 0.3 | 0.4 | 0.2 | 0.6 | 0.6 | 0.5 | 0.7 | 0.2 | 1338 |
| YKHWPQIAQFAPSAS | 0.5 | 0.3 | 0.5 | 0.3 | 0.7 | 0.7 | 0.5 | 0.6 | 0.4 | 1339 |
| KHWPQIAQFAPSASA | 0.3 | 0.1 | 0.3 | 0.3 | 0.2 | 0.4 | 0.5 | 0.2 | 0.4 | 1340 |
| HWPQIAQFAPSASAF | 0.5 | 0.3 | 0.5 | 0.4 | 0.8 | 1.0 | 0.6 | 0.6 | 0.6 | 1341 |
| WPQIAQFAPSASAFF | 0.5 | 0.3 | 0.5 | 0.3 | 0.8 | 0.9 | 0.5 | 0.6 | 0.5 | 1342 |
| PQIAQFAPSASAFFG | 0.4 | 0.3 | 0.4 | 0.3 | 0.7 | 0.7 | 0.5 | 0.5 | 0.4 | 1343 |
| QIAQFAPSASAFFGM | 0.5 | 0.3 | 0.5 | 0.3 | 0.8 | 0.8 | 0.5 | 0.5. | 0.5 | 1344 |
| IAQFAPSASAFFGMS | 0.4 | 0.2 | 0.4 | 0.3 | 0.7 | 0.6 | 0.4 | 0.4 | 0.4 | 1345 |
| AQFAPSASAFFGMSR | 0.3 | 0.2 | 0.6 | 0.5 | 0.8 | 0.6 | 2.1 | 0.4 | 0.2 | 1346 |
| QFAPSASAFFGMSRI | 0.4 | 0.2 | 0.4 | 0.2 | 0.6 | 0.7 | 0.4 | 0.3 | 0.5 | 1347 |
| FAPSASAFFGMSRIG | 0.3 | 0.1 | 0.4 | 0.3 | 0.5 | 0.4 | 0.9 | 0.5 | 0.0 | 1348 |
| APSASAFFGMSRIGM | 0.4 | 0.3 | 0.7 | 0.4 | 0.7 | 0.9 | 2.0 | 0.6 | 0.5 | 1349 |
| PSASAFFGMSRIGME | 0.4 | 0.3 | 0.3 | 0.2 | 0.6 | 0.4 | 0.3 | 0.4 | 0.3 | 1350 |
| SASAFFGMSRIGMEV | 0.5 | 0.3 | 0.6 | 0.5 | 0.8 | 0.6 | 1.6 | 0.9 | 0.4 | 1351 |
| ASAFFGMSRIGMEVT | 0.3 | 0.2 | 0.3 | 0.1 | 0.5 | 0.4 | 0.3 | 0.6 | 0.3 | 1352 |
| SAFFGMSRIGMEVTP | 0.4 | 0.3 | 0.4 | 0.2 | 0.5 | 0.4 | 0.4 | 0.5 | 0.4 | 1353 |
| AFFGMSRIGMEVTPS | 0.4 | 0.3 | 0.4 | 0.2 | 0.6 | 0.6 | 0.4 | 0.5 | 0.4 | 1354 |
| EFGMSRIGMEVTPSG | 0.3 | 0.2 | 0.4 | 0.1 | 0.5 | 0.4 | 0.3 | 0.4 | 0.1 | 1355 |
| FGMSRIGMEVTPSGT | 0.3 | 0.2 | 0.4 | 0.2 | 0.4 | 0.3 | 0.3 | 0.3 | 0.2 | 1356 |
| GMSRIGMEVTPSGTW | 0.3 | 0.2 | 0.3 | 0.2 | 0.5 | 0.5 | 0.4 | 0.5 | 0.3 | 1357 |
| MSRIGMEVTPSGTWL | 0.4 | 0.3 | 0.5 | 0.3 | 0.6 | 0.7 | 0.5 | 0.5 | 0.7 | 1358 |
| SRIGMEVTPSGTWLT | 0.3 | 0.2 | 0.4 | 0.2 | 0.5 | 0.5 | 0.4 | 0.4 | 0.5 | 1359 |
| RIGMEVTPSGTWLTY | 0.4 | 0.2 | 0.5 | 0.3 | 0.7 | 0.8 | 0.6 | 0.5 | 0.5 | 1360 |
| IGMEVTPSGTWLTYH | 0.5 | 0.3 | 0.5 | 0.3 | 0.8 | 1.0 | 0.6 | 0.5 | 0.7 | 1361 |
| GMEVTPSGTWLTYHG | 0.4 | 0.2 | 0.4 | 0.3 | 0.7 | 0.8 | 0.5 | 0.5 | 0.4 | 1362 |
| MEVTPSGTWLTYHGA | 0.4 | 0.3 | 0.5 | 0.3 | 0.7 | 0.8 | 0.5 | 0.4 | 0.5 | 1363 |
| EVTPSGTWLTYHGAI | 0.4 | 0.3 | 0.5 | 0.3 | 0.8 | 0.9 | 0.5 | 0.4 | 0.7 | 1364 |
| VTPSGTWLTYHGAIK | 0.2 | 0.1 | 0.2 | 0.1 | 0.4 | 0.3 | 0.3 | 0.2 | 0.0 | 1365 |
| TPSGTWLTYHGAIKL | 0.4 | 0.2 | 0.4 | 0.2 | 0.8 | 0.8 | 0.5 | 0.4 | 0.3 | 1366 |
| PSGTWLTYHGAIKLD | 0.2 | 0.1 | 0.2 | 0.1 | 0.4 | 0.3 | 0.3 | 0.1 | 0.1 | 1367 |
| SGTWLTYHGAIKLDD | 0.8 | 0.5 | 0.7 | 0.4 | 1.0 | 1.2 | 0.8 | 1.6 | 1.8 | 1368 |
| GTWLTYHGAIKLDDK | 0.2 | 0.1 | 0.2 | 0.1 | 0.4 | 0.3 | 0.3 | 0.5 | 0.1 | 1369 |
| TWLTYHGAIKLDDKD | 0.2 | 0.2 | 0.2 | 0.1 | 0.4 | 0.3 | 0.2 | 0.3 | 0.3 | 1370 |
| WLTYHGAIKLDDKDP | 0.2 | 0.1 | 0.2 | 0.1 | 0.4 | 0.3 | 0.3 | 0.2 | 0.0 | 1371 |
| LTYHGAIKLDDKDPQ | 0.3 | 0.1 | 0.3 | 0.1 | 0.5 | 0.3 | 0.4 | 0.4 | 0.1 | 1372 |
| TYHGAIKLDDKDPQF | 0.2 | 0.1 | 0.2 | 0.1 | 0.3 | 0.2 | 0.3 | 0.2 | 0.0 | 1373 |
| YHGAIKLDDKDPQFK | 0.2 | 0.1 | 0.2 | 0.1 | 0.4 | 0.3 | 0.3 | 0.1 | 0.1 | 1374 |
| HGAIKLDDKDPQFKD | 0.2 | 0.1 | 0.2 | 0.1 | 0.5 | 0.3 | 0.3 | 0.3 | 0.3 | 1375 |
| GAIKLDDKDPQFKDN | 0.3 | 0.2 | 0.4 | 0.2 | 0.5 | 0.5 | 0.4 | 0.5 | 0.8 | 1376 |
| AIKLDDKDPQFKDNV | 0.3 | 0.2 | 0.3 | 0.2 | 0.5 | 0.4 | 0.4 | 0.2 | 0.4 | 1377 |
| 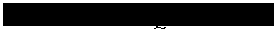 | 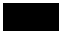 | 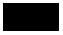 | 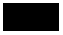 | 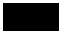 | 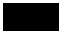 | 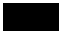 | 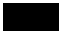 | 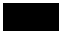 | 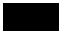 | 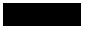 |
| 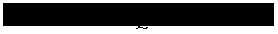 | 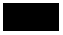 | 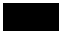 | 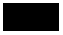 | 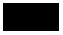 | 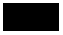 | 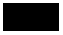 | 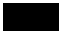 | 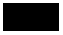 | 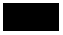 | 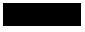 |
| 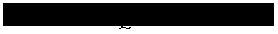 | 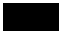 | 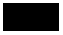 | 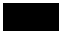 | 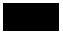 | 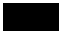 | 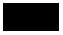 | 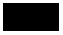 | 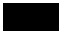 | 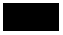 | 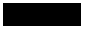 |
| 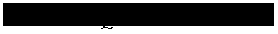 | 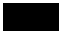 | 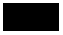 | 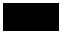 | 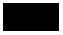 | 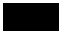 | 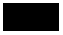 | 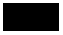 | 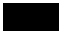 | 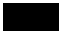 | 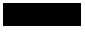 |
| 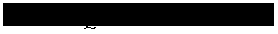 | 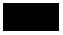 | 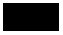 | 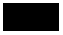 | 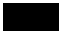 | 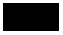 | 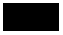 | 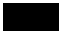 | 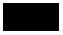 | 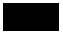 | 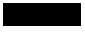 |
| 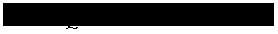 | 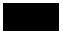 | 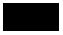 | 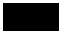 | 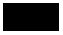 | 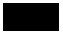 | 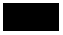 | 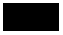 | 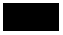 | 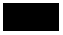 | 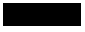 |
| 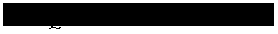 | 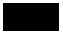 | 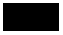 | 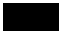 | 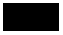 | 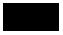 | 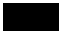 | 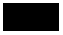 | 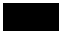 | 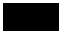 | 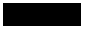 |
| 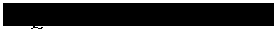 | 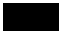 | 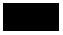 | 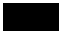 | 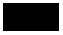 | 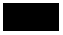 | 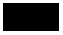 | 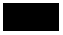 | 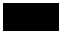 | 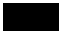 | 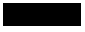 |
| 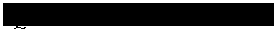 | 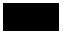 | 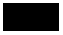 | 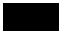 | 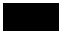 | 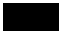 | 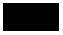 | 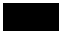 | 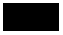 | 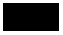 | 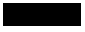 |
| FKDNVILLNKHIDAY | 0.5 | 0.3 | 0.5 | 0.3 | 0.8 | 0.8 | 0.5 | 0.5 | 0.2 | 1378 |
| KDNVILLNKHIDAYK | 0.3 | 0.2 | 0.2 | 0.1 | 0.4 | 0.3 | 0.3 | 0.4 | 0.1 | 1379 |
| DNVILLNKHIDAYKT | 0.4 | 0.3 | 0.4 | 0.2 | 0.7 | 0.7 | 0.5 | 0.8 | 0.3 | 1380 |
| NVILLNKHTDAYKTF | 0.5 | 0.3 | 0.4 | 0.3 | 0.7 | 0.7 | 0.9 | 0.7 | 0.3 | 1381 |
| VILLNKHIDAYKTFP | 0.3 | 0.1 | 0.3 | 0.2 | 0.5 | 0.4 | 0.4 | 0.3 | 0.2 | 1382 |
| ILLNKHIDAYKTFPP | 0.4 | 0.3 | 0.5 | 0.3 | 0.7 | 0.7 | 0.5 | 0.7 | 0.5 | 1383 |
| LLNKHIDAYKTFPPT | 0.3 | 0.1 | 0.3 | 0.2 | 0.4 | 0.3 | 0.3 | 0.4 | 0.3 | 1384 |
| LNKHIDAYKTFPPTE | 0.2 | 0.1 | 0.2 | 0.1 | 0.4 | 0.3 | 0.3 | 0.1 | 0.2 | 1385 |
| NKHIDAYKTFPPTEP | 0.3 | 0.1 | 0.2 | 0.1 | 0.4 | 0.3 | 0.3 | 0.2 | 0.3 | 1386 |
| KHIDAYKTFPPTEPK | 0.2 | 0.1 | 0.2 | 0.1 | 0.4 | 0.3 | 0.3 | 0.1 | 0.1 | 1387 |
| HIDAYKTFPPTEPKK | 0.2 | 0.1 | 0.2 | 0.1 | 0.3 | 0.2 | 0.3 | 0.0 | 0.0 | 1388 |
| IDAYKTFPPTEPKKD | 0.2 | 0.1 | 0.2 | 0.1 | 0.4 | 0.3 | 0.3 | 0.0 | 0.0 | 1389 |
| DAYKTFPPTEPKKDK | 0.2 | 0.1 | 0.2 | 0.1 | 0.3 | 0.2 | 0.2 | 0.1 | 0.0 | 1390 |
| AYKTFPPTEPKKDKK | 0.1 | 0.1 | 0.2 | 0.0 | 0.2 | 0.1 | 0.1 | 0.0 | 0.0 | 1391 |
| YKTFPPTEPKKDKKK | 0.2 | 0.1 | 0.1 | 0.0 | 0.4 | 0.2 | 0.3 | 0.2 | 0.0 | 1392 |
| KTFPPTEPKKDKKKK | 0.2 | 0.1 | 0.2 | 0.0 | 0.4 | 0.2 | 0.2 | 0.2 | 0.0 | 1393 |
| TFPPTEPKKDKKKKT | 0.2 | 0.1 | 0.3 | 0.1 | 0.4 | 0.4 | 0.3 | 0.3 | 0.1 | 1394 |
| FPPTEPKKDKKKKTD | 0.2 | 0.1 | 0.2 | 0.1 | 0.4 | 0.2 | 0.2 | 0.1 | 0.0 | 1395 |
| PPTEPKKDKKKKTDE | 0.2 | 0.1 | 0.2 | 0.1 | 0.4 | 0.2 | 0.2 | 0.2 | 0.0 | 1396 |
| PTEPKKDKKKKTDEA | 0.2 | 0.1 | 0.3 | 0.1 | 0.3 | 0.3 | 0.3 | 0.2 | 0.0 | 1397 |
| TEPKKDKKKKTDEAQ | 0.2 | 0.1 | 1.9 | 0.1 | 0.4 | 0.3 | 0.3 | 0.2 | 0.0 | 1398 |
| EPKKDKKKKTDEAQP | 0.2 | 0.1 | 2.1 | 0.1 | 0.4 | 0.3 | 0.3 | 0.1 | 0.0 | 1399 |
| PKKDKKKKTDEAQPL | 0.2 | 0.1 | 0.2 | 0.1 | 0.4 | 0.3 | 0.3 | 0.2 | 0.1 | 1400 |
| KKDKKKKTDEAQPLP | 0.2 | 0.1 | 2.3 | 0.2 | 0.3 | 0.3 | 0.3 | 0.1 | 0.1 | 1401 |
| KDKKKKTDEAQPLPQ | 0.2 | 0.1 | 0.2 | 0.2 | 0.4 | 0.3 | 0.4 | 0.1 | 0.1 | 1402 |
| DKKKKTDEAQPLPQR | 0.2 | 0.1 | 0.2 | 0.1 | 0.4 | 0.3 | 0.3 | 0.3 | 0.3 | 1403 |
| KKKKTDEAQPLPQRQ | 0.2 | 0.1 | 0.3 | 0.1 | 0.4 | 0.3 | 0.3 | 0.2 | 0.1 | 1404 |
| KKKTDEAQPLPQRQK | 0.2 | 0.1 | 0.1 | 0.0 | 0.3 | 0.2 | 0.3 | 0.0 | 0.0 | 1405 |
| KKTDEAQPLPQRQKK | 0.2 | 0.1 | 0.1 | 0.0 | 0.4 | 0.2 | 0.2 | 0.0 | 0.0 | 1406 |
| KTDEAQPLPQRQKKQ | 0.3 | 0.1 | 0.3 | 0.6 | 0.3 | 0.1 | 0.7 | 0.5 | 0.2 | 1407 |
| TDEAQPLPQRQKKQP | 0.2 | 0.0 | 0.2 | 0.1 | 0.1 | 0.0 | 0.1 | 0.1 | 0.0 | 1408 |
| DEAQPLPQRQKKQPT | 0.2 | 0.1 | 0.2 | 0.1 | 0.3 | 0.2 | 0.3 | 0.2 | 0.0 | 1409 |
| EAQPLPQRQKKQPTV | 0.2 | 0.1 | 0.2 | 0.2 | 0.3 | 0.2 | 0.3 | 0.5 | 0.2 | 1410 |
| AQPLPQRQKKQPTVT | 0.2 | 0.1 | 0.2 | 0.1 | 0.3 | 0.2 | 0.2 | 0.2 | 0.3 | 1411 |
| 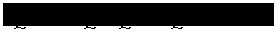 | 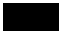 | 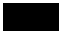 | 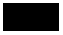 | 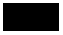 | 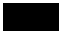 | 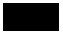 | 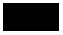 | 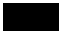 | 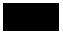 | 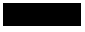 |
| 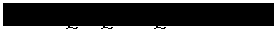 | 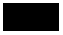 | 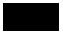 | 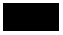 | 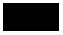 | 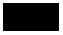 | 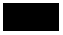 | 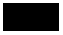 | 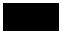 | 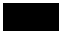 | 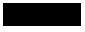 |
| 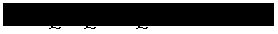 | 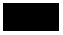 | 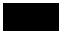 | 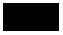 | 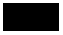 | 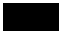 | 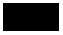 | 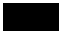 | 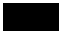 | 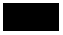 | 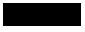 |
| 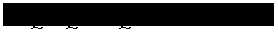 | 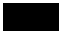 | 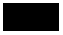 | 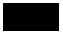 | 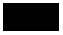 | 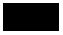 | 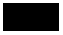 | 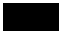 | 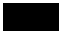 | 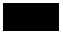 | 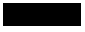 |
| 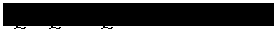 | 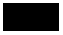 | 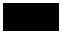 | 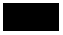 | 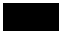 | 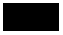 | 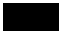 | 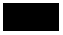 | 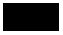 | 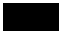 | 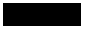 |
| 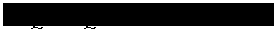 | 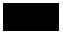 | 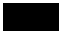 | 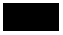 | 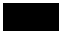 | 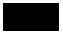 | 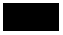 | 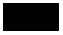 | 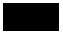 | 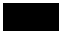 | 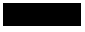 |
| 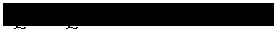 | 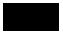 | 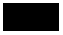 | 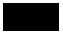 | 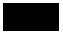 | 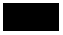 | 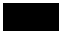 | 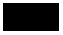 | 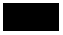 | 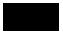 | 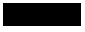 |
| KKQPTVTLLPAADMD | 0.3 | 0.2 | 0.2 | 0.1 | 0.4 | 0.2 | 0.2 | 0.2 | 0.7 | 1412 |
| KQPTVTLLPAADMDD | 0.3 | 0.2 | 0.2 | 0.0 | 0.4 | 0.3 | 0.2 | 0.3 | 0.6 | 1413 |
| QPTVTLLPAADMDDF | 0.3 | 0.2 | 0.2 | 0.1 | 0.5 | 0.3 | 0.2 | 0.4 | 0.4 | 1414 |
| PTVTLLPAADMDDFS | 0.2 | 0.2 | 0.3 | 0.1 | 0.4 | 0.2 | 0.2 | 0.1 | 0.7 | 1415 |
| TVTLLPAADMDDFSR | 0.3 | 0.2 | 0.2 | 0.0 | 0.4 | 0.1 | 0.2 | 0.2 | 0.5 | 1416 |
| VTLLPAADMDDFSRQ | 0.3 | 0.1 | 0.2 | 0.0 | 0.1 | 0.4 | 0.1 | 0.2 | 0.2 | 1417 |
| TLLPAADMDDFSRQL | 0.4 | 0.2 | 0.5 | 0.1 | 0.4 | 0.1 | 0.2 | 0.4 | 0.0 | 1418 |
| LLPAADMDDFSRQLQ | 0.3 | 0.1 | 0.4 | 0.1 | 0.5 | 0.2 | 0.3 | 0.3 | 0.2 | 1419 |
| LPAADMDDFSRQLQN | 0.3 | 0.2 | 0.5 | 0.4 | 0.6 | 0.4 | 0.3 | 0.6 | 0.5 | 1420 |
| PAADMDDFSRQLQNS | 0.3 | 0.1 | 0.3 | 1.1 | 0.6 | 0.3 | 0.3 | 0.3 | 0.3 | 1421 |
| AADMDDFSRQLQNSM | 0.3 | 0.2 | 0.3 | 1.6 | 0.5 | 0.3 | 0.2 | 0.2 | 0.2 | 1422 |
| ADMDDFSRQLQNSMS | 0.3 | 0.2 | 0.2 | 0.0 | 0.4 | 0.3 | 0.2 | 0.2 | 0.1 | 1423 |
| DMDDFSRQLQNSMSG | 0.3 | 0.1 | 0.3 | 0.0 | 0.4 | 0.2 | 0.2 | 0.2 | 0.0 | 1424 |
| MDDFSRQLQNSMSGA | 0.4 | 0.1 | 0.4 | 0.1 | 0.4 | 0.4 | 0.3 | 0.2 | 0.3 | 1425 |
| DDFSRQLQNSMSGAS | 0.3 | 0.1 | 0.3 | 0.1 | 0.4 | 0.3 | 0.2 | 0.1 | 0.2 | 1426 |
| DFSRQLQNSMSGASA | 0.3 | 0.1 | 0.4 | 0.2 | 0.4 | 0.2 | 0.3 | 0.2 | 0.4 | 1427 |
| FSRQLQNSMSGASAD | 0.2 | 0.1 | 0.2 | 0.1 | 0.4 | 0.2 | 0.2 | 0.0 | 0.0 | 1428 |
| SRQLQNSMSGASADS | 0.2 | 0.1 | 0.3 | 0.1 | 0.4 | 0.3 | 0.2 | 0.2 | 0.3 | 1429 |
| RQLQNSMSGASADST | 0.2 | 0.1 | 0.3 | 0.1 | 0.4 | 0.2 | 0.2 | 0.2 | 0.1 | 1430 |
| QLQNSMSGASADSTQ | 0.3 | 0.1 | 0.4 | 0.1 | 0.4 | 0.1 | 0.3 | 0.1 | 0.1 | 1431 |
| LQNSMSGASADSTQA | 0.2 | 0.1 | 0.4 | 0.0 | 0.5 | 0.1 | 0.1 | 0.0 | 0.0 | 1432 |

TABLE 17
Binding of two control sera to linear and looped/cyclic
peptides of the protein X1 of SARS-CoV Urbani.

| | Control | Control serum | Control | Control Serum | |
|---|---|---|---|---|---|
| | serum LUMC | Blood-bank | serum LUMC | Blood-Bank | |
| Peptide | linear | linear | looped | Looped | SEQ |
| sequence | peptides | peptides | peptides | peptides | ID NO |
| MDLFMRFFTLGSITA | 0.6 | 0.7 | 0.6 | 0.6 | 607 |
| DLFMRFFTLGSITAQ | 0.6 | 0.6 | 0.5 | 0.5 | 608 |
| LFMRFFTLGSITAQP | 0.6 | 0.7 | 0.5 | 0.7 | 609 |
| FMRFFTLGSITAQPV | 0.7 | 0.7 | 0.6 | 0.6 | 610 |
| MRFFTLGSITAQPVK | 0.7 | 0.4 | 0.4 | 0.2 | 611 |
| RFFTLGSITAQPVKI | 1.1 | 1.0 | 0.7 | 0.8 | 9 |
| FFTLGSITAQPVKID | 0.5 | 0.5 | 0.3 | 0.2 | 10 |
| FTLGSITAQPVKIDN | 0.6 | 0.5 | 1.2 | 1.6 | 11 |
| TLGSITAQPVKTDNA | 0.7 | 0.5 | 0.6 | 0.6 | 12 |
| LGSITAQPVKIDNAS | 0.5 | 0.4 | 0.4 | 0.4 | 13 |
| GSITAQPVKIDNASP | 0.6 | 0.6 | 0.5 | 0.6 | 14 |
| SITAQPVKIDNASPA | 0.6 | 0.6 | 0.4 | 0.4 | 15 |
| ITAQPVKIDNASPAS | 0.7 | 0.7 | 0.5 | 0.5 | 16 |
| TAQPVKIDNASPAST | 0.6 | 0.7 | 0.6 | 0.5 | 17 |
| AQPVKIDNASPASTV | 0.6 | 0.7 | 1.0 | 1.0 | 18 |
| QPVKIDNASPASTVH | 0.6 | 0.6 | 0.6 | 0.6 | 19 |
| PVKIDNASPASTVHA | 0.5 | 0.7 | 0.9 | 0.7 | 20 |
| VKIDNASPASTVHAT | 0.7 | 0.7 | 0.6 | 0.5 | 21 |
| KIDNASPASTVHATA | 0.7 | 0.6 | 0.7 | 0.6 | 22 |
| IDNASPASTVHATAT | 0.7 | 0.8 | 0.6 | 0.7 | 23 |
| 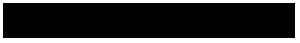 | 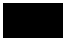 | 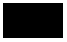 | 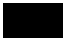 | 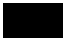 | 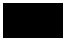 |
| 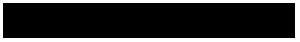 | 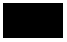 | 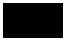 | 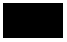 | 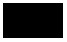 | 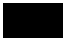 |
| 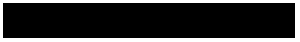 | 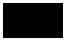 | 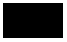 | 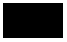 | 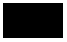 | 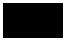 |
| 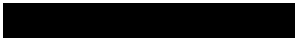 | 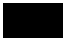 | 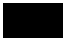 | 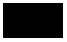 | 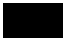 | 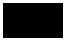 |
| 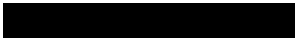 | 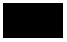 | 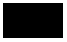 | 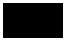 | 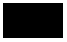 | 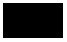 |
| 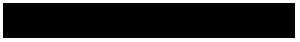 | 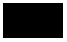 | 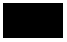 | 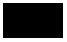 | 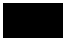 | 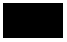 |
| 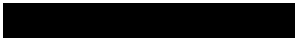 | 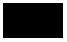 | 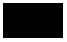 | 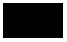 | 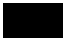 | 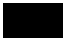 |
| 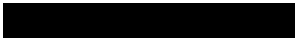 | 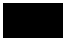 | 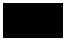 | 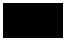 | 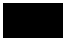 | 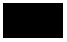 |
| 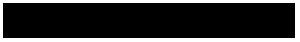 | 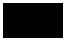 | 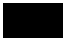 | 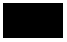 | 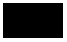 | 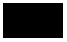 |
| HATATIPLQASLPFG | 0.6 | 0.8 | 0.7 | 0.7 | 612 |
| ATATIPLQASLPFGW | 0.7 | 0.9 | 0.7 | 0.8 | 613 |
| TATIPLQASLPFGWL | 0.8 | 1.0 | 0.8 | 0.8 | 614 |
| ATIPLQASLPFGWLV | 0.5 | 0.8 | 0.7 | 0.7 | 615 |
| TIPLQASLPFGWLVI | 0.7 | 0.8 | 0.7 | 0.7 | 616 |
| IPLQASLPFGWLVIG | 0.8 | 0.7 | 0.6 | 0.6 | 617 |
| PLQASLPFGWLVIGV | 0.9 | 0.8 | 0.5 | 0.7 | 618 |
| LQASLPFGWLVIGVA | 0.5 | 0.8 | 0.5 | 0.6 | 619 |
| QASLPFGWLVIGVAF | 0.6 | 0.7 | 0.4 | 0.4 | 620 |
| ASLPFGWLVIGVAFL | 0.6 | 0.6 | 0.6 | 0.4 | 621 |
| SLPFGWLVIGVAFLA | 0.6 | 0.6 | 0.3 | 0.4 | 622 |
| LPFGWLVIGVAFLAV | 0.7 | 0.7 | 0.4 | 0.5 | 623 |
| PFGWLVIGVAFLAVF | 0.5 | 0.5 | 0.5 | 0.5 | 624 |
| FGWLVIGVAFLAVFQ | 0.5 | 0.5 | 0.7 | 0.6 | 625 |
| GWLVIGVAFLAVFQS | 0.6 | 0.5 | 0.7 | 0.8 | 626 |
| WLVIGVAFLAVFQSA | 0.6 | 0.6 | 0.5 | 0.5 | 627 |
| LVIGVAFLAVFQSAT | 0.6 | 0.6 | 1.0 | 1.1 | 628 |
| VIGVAFLAVFQSATK | 0.5 | 0.5 | 0.5 | 0.5 | 629 |
| IGVAFLAVPQSATKI | 0.8 | 0.8 | 0.5 | 0.5 | 630 |
| GVAFLAVFQSATKII | 0.7 | 0.5 | 0.7 | 0.7 | 631 |
| VAFLAVFQSATKIIA | 0.5 | 0.6 | 0.7 | 0.7 | 632 |
| AFLAVFQSATKIIAL | 0.5 | 0.5 | 0.6 | 0.7 | 633 |
| FLAVFQSATKIIALN | 0.6 | 0.6 | 0.7 | 0.6 | 634 |
| LAVFQSATKIIALNK | 0.6 | 0.7 | 0.6 | 0.5 | 635 |
| 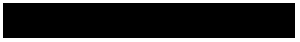 | 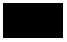 | 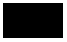 | 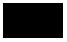 | 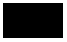 | 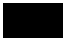 |
| 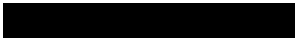 | 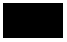 | 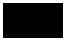 | 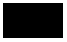 | 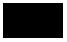 | 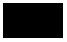 |
| 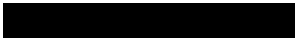 | 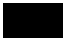 | 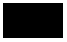 | 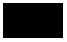 | 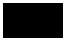 | 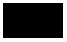 |
| 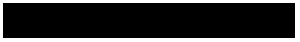 | 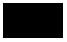 | 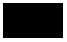 | 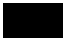 | 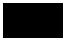 | 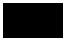 |
| 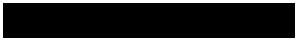 | 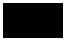 | 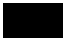 | 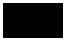 | 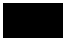 | 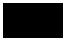 |
| 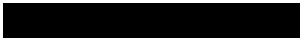 | 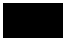 | 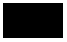 | 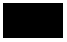 | 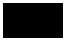 | 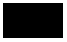 |
| 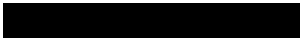 | 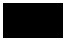 | 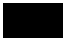 | 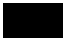 | 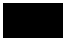 | 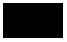 |
| 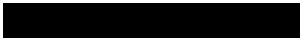 | 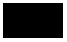 | 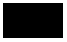 | 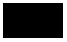 | 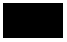 | 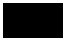 |
| 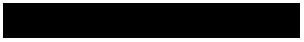 | 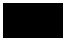 | 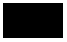 | 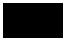 | 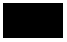 | 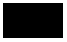 |
| 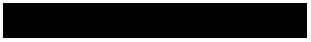 | 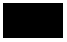 | 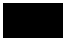 | 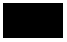 | 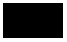 | 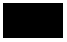 |
| ALNKRWQLALYKGFQ | 0.6 | 0.6 | 0.8 | 0.9 | 502 |
| LNKRWQLALYKGFQF | 0.6 | 0.8 | 0.6 | 0.6 | 503 |
| NKRWQLALYKGFQFI | 0.6 | 0.6 | 0.7 | 0.7 | 504 |
| KRWQLALYKGFQFIC | 0.5 | 0.6 | 0.7 | 0.7 | 636 |
| RWQLALYKGFQFICN | 0.4 | 0.6 | 0.6 | 0.6 | 637 |
| WQLALYKGFQFTCNL | 0.6 | 0.6 | 0.7 | 0.6 | 638 |
| QLALYKGFQFICNLL | 0.5 | 0.6 | 0.6 | 0.5 | 639 |
| LALYKGFQFICNLLL | 0.5 | 0.6 | 0.6 | 0.6 | 640 |
| ALYKGFQFICNLLLL | 0.5 | 0.6 | 0.5 | 0.5 | 641 |
| LYKGFQFICNLLLLF | 0.5 | 0.5 | 0.6 | 0.4 | 642 |
| YKGFQFICNLLLLFV | 0.8 | 1.0 | 0.4 | 0.4 | 643 |
| KGFQFICNLLLLFVT | 0.7 | 0.7 | 0.5 | 0.5 | 644 |
| GFQFICNLLLLFVTI | 0.5 | 0.4 | 0.5 | 0.4 | 645 |
| FQFICNLLLLFVTIY | 0.5 | 0.5 | 0.3 | 0.4 | 646 |
| QFICNLLLLFVTIYS | 0.6 | 0.6 | 0.5 | 0.5 | 647 |
| FICNLLLLFVTTYSH | 0.5 | 0.6 | 0.5 | 0.5 | 648 |
| ICNLLLLFVTIYSHL | 0.5 | 0.5 | 0.4 | 0.5 | 649 |
| CNLLLLFVTIYSHLL | 0.4 | 0.4 | 0.5 | 0.5 | 650 |
| NLLLLFVTIYSHLLL | 0.5 | 0.4 | 0.6 | 0.5 | 651 |
| LLLLFVTIYSHLLLV | 0.6 | 0.5 | 0.5 | 0.6 | 652 |
| LLLFVTIYSHLLLVA | 0.4 | 0.4 | 0.5 | 0.5 | 653 |
| LLFVTIYSHLLLVAA | 0.4 | 0.5 | 0.6 | 0.4 | 654 |
| LFVTIYSHLLLVAAG | 0.4 | 0.5 | 0.6 | 0.6 | 655 |
| FVTIYSHLLLVAAGM | 0.5 | 0.6 | 0.5 | 0.6 | 656 |
| VTIYSHLLLVAAGME | 0.5 | 0.5 | 0.6 | 0.4 | 657 |
| TIYSHLLLVAAGMEA | 0.4 | 0.4 | 0.6 | 0.5 | 658 |
| IYSHLLLVAAGMEAQ | 0.5 | 0.5 | 0.5 | 0.4 | 659 |
| YSHLLLVAAGMEAQF | 0.5 | 0.6 | 0.6 | 0.4 | 660 |
| SHLLLVAAGMEAQFL | 0.7 | 0.7 | 0.2 | 0.4 | 661 |
| HLLLVAAGMEAQFLY | 0.7 | 0.6 | 0.4 | 0.6 | 662 |
| LLLVAAGMEAQFLYL | 1.0 | 0.6 | 0.7 | 0.5 | 663 |
| LLVAAGMEAQFLYLY | 0.7 | 0.5 | 0.6 | 0.5 | 664 |
| LVAAGMEAQFLYLYA | 1.1 | 0.4 | 0.6 | 0.5 | 665 |
| VAAGMEAQFLYLYAL | 0.9 | 0.5 | 0.8 | 0.5 | 666 |
| AAGMEAQFLYLYALI | 0.9 | 0.5 | 0.7 | 0.5 | 667 |
| AGMEAQFLYLYALIY | 0.6 | 0.5 | 0.6 | 0.5 | 668 |
| GMEAQFLYLYALIYF | 0.6 | 0.4 | 0.8 | 0.5 | 669 |
| MEAQFLYLYALIYFL | 0.5 | 0.2 | 0.5 | 0.4 | 670 |
| EAQFLYLYALIYFLQ | 0.5 | 0.3 | 0.5 | 0.4 | 671 |
| AQFLYLYALIYFLQC | 0.5 | 0.4 | 0.5 | 0.4 | 672 |
| QFLYLYALIYFLQCI | 0.4 | 0.5 | 0.6 | 0.5 | 673 |
| FLYLYALIYFLQCIN | 0.5 | 0.5 | 0.4 | 0.5 | 674 |
| LYLYALIYFLQCINA | 0.5 | 0.5 | 0.4 | 0.5 | 675 |
| YLYALIYFLQCINAC | 0.6 | 0.5 | 0.5 | 0.5 | 676 |
| LYALIYFLQCINACR | 0.6 | 0.7 | 0.6 | 0.4 | 677 |
| YALIYFLQCINACRI | 0.6 | 0.6 | 0.2 | 0.3 | 678 |
| ALIYFLQCINACRII | 0.5 | 0.5 | 0.3 | 0.5 | 679 |
| LIYFLQCINACRIIM | 0.6 | 0.6 | 0.7 | 0.9 | 680 |
| IYFLQCINACRIIMR | 0.7 | 0.7 | 0.6 | 0.6 | 681 |
| YFLQCINACRIIMRC | 0.7 | 0.8 | 0.6 | 0.6 | 682 |
| FLQCINACRIIMRCW | 0.9 | 0.8 | 0.6 | 0.6 | 683 |
| LQCINACRIIMRCWL | 0.7 | 0.9 | 0.6 | 0.7 | 505 |
| QCINACRIIMRCWLC | 0.7 | 0.9 | 0.7 | 0.6 | 506 |
| CINACRIIMRCWLCW | 0.8 | 0.7 | 0.7 | 0.8 | 507 |
| INACRIIMRCWLCWK | 0.4 | 0.9 | 0.6 | 0.7 | 33 |
| NACRIIMRCWLCWKC | 0.5 | 1.0 | 0.6 | 0.8 | 34 |
| ACRIIMRCWLCWKCK | 0.8 | 1.0 | 0.3 | 0.1 | 35 |
| CRIIMRCWLCWKCKS | 0.3 | 0.4 | 0.7 | 0.7 | 36 |
| RIIMRCWLCWKCKSK | 0.2 | 0.3 | 0.3 | 0.3 | 37 |
| IIMRCWLCWKCKSKN | 0.4 | 0.5 | 0.6 | 0.6 | 38 |
| IMRCWLCWKCKSKNP | 0.5 | 0.5 | 0.2 | 0.4 | 39 |
| MRCWLCWKCKSKNPL | 0.8 | 0.9 | 0.4 | 0.2 | 40 |
| RCWLGWKCKSKNPLL | 1.0 | 1.2 | 0.7 | 0.7 | 41 |
| CWLCWKCKSKNPLLY | 0.7 | 0.9 | 0.7 | 0.6 | 42 |
| WLCWKCKSKNPLLYD | 0.7 | 0.6 | 0.8 | 0.7 | 43 |
| LCWKCKSKNPLLYDA | 0.7 | 0.7 | 0.8 | 0.8 | 44 |
| CWKCKSKNPLLYDAN | 0.8 | 0.7 | 0.7 | 0.7 | 45 |
| WKCKSKNPLLYDANY | 0.8 | 0.7 | 0.5 | 0.6 | 684 |
| KCKSKNPLLYDANYF | 0.5 | 0.6 | 0.7 | 0.7 | 685 |
| CKSKNPLLYDANYFV | 0.8 | 0.5 | 0.8 | 0.6 | 686 |
| KSKNPLLYDANYFVC | 0.7 | 0.3 | 0.6 | 0.5 | 687 |
| SKNPLLYDANYFVCW | 0.5 | 0.4 | 0.6 | 0.6 | 688 |
| KNPLLYDANYFVCWH | 0.4 | 0.4 | 0.7 | 0.6 | 689 |
| NPLLYDANYFVCWHT | 0.6 | 0.6 | 0.6 | 0.7 | 690 |
| PLLYDANYFVCWHTH | 0.6 | 0.7 | 0.6 | 0.6 | 691 |
| LLYDANYFVCWHTHN | 0.7 | 0.8 | 0.5 | 0.6 | 692 |
| LYDANYFVCWHTHNY | 0.7 | 0.8 | 0.5 | 0.7 | 693 |
| YDANYFVCWHTHNYD | 0.7 | 0.7 | 0.4 | 0.3 | 46 |
| DANYFVCWHTHNYDY | 0.8 | 0.8 | 1.0 | 0.5 | 47 |
| ANYFVCWHTHNYDYC | 0.7 | 0.7 | 0.6 | 0.5 | 48 |
| NYFVCWHTHNYDYCI | 0.6 | 0.6 | 0.6 | 0.6 | 49 |
| YFVCWHTHNYDYCIP | 0.7 | 0.6 | 0.6 | 0.6 | 50 |
| FVCWHTHNYDYCIPY | 0.7 | 0.6 | 0.6 | 0.5 | 51 |
| VCWHTHNYDYCIPYN | 0.8 | 0.7 | 0.6 | 0.7 | 52 |
| CWHTHNYDYCIPYNS | 0.7 | 0.6 | 0.6 | 0.6 | 53 |
| WHTHNYDYCIPYNSV | 0.8 | 0.7 | 0.9 | 0.7 | 54 |
| HTHNYDYCIPYNSVT | 0.7 | 0.6 | 0.6 | 0.6 | 55 |
| THNYDYCIPYNSVTD | 0.6 | 0.5 | 0.7 | 0.7 | 56 |
| HNYDYCIPYNSVTDT | 0.4 | 0.3 | 0.6 | 0.7 | 57 |
| NYDYCIPYNSVTDTI | 0.6 | 0.6 | 0.7 | 0.6 | 58 |
| YDYCIPYNSVTDTIV | 0.7 | 0.6 | 0.6 | 0.5 | 59 |
| DYCIPYNSVTDTIVV | 0.6 | 0.7 | 0.9 | 0.7 | 60 |
| YCIPYNSVTDTIVVT | 0.7 | 0.8 | 0.7 | 0.7 | 61 |
| CIPYNSVTDTIVVTE | 0.6 | 0.5 | 0.5 | 0.6 | 694 |
| IPYNSVTDTIVVTEG | 0.5 | 0.4 | 0.4 | 0.6 | 695 |
| PYNSVTDTIVVTEGD | 0.3 | 0.4 | 0.4 | 0.4 | 696 |
| YNSVTDTIVVTEGDG | 0.4 | 0.4 | 0.4 | 0.3 | 697 |
| NSVTDTIVVTEGDGI | 0.4 | 0.3 | 0.5 | 0.4 | 698 |
| SVTDTIVVTEGDGIS | 0.4 | 0.4 | 0.3 | 0.3 | 699 |
| VTDTIVVTEGDGIST | 0.4 | 0.4 | 0.4 | 0.4 | 700 |
| TDTIVVTEGDGISTP | 0.5 | 0.5 | 0.4 | 0.4 | 701 |
| DTIVVTEGDGISTPK | 0.3 | 0.4 | 0.3 | 0.4 | 702 |
| TIVVTEGDGISTPKL | 0.5 | 0.5 | 0.7 | 0.6 | 703 |
| IVVTEGDGISTPKLK | 0.3 | 0.3 | 0.3 | 0.3 | 704 |
| VVTEGDGISTPKLKE | 0.2 | 0.3 | 0.3 | 0.3 | 705 |
| VTEGDGISTPKLKED | 0.2 | 0.2 | 0.3 | 0.3 | 706 |
| TEGDGISTPKLKEDY | 0.2 | 0.2 | 0.5 | 0.5 | 707 |
| EGDGISTPKLKEDYQ | 0.3 | 0.2 | 0.4 | 0.4 | 708 |
| GDGISTPKLKEDYQI | 0.5 | 0.6 | 0.4 | 0.4 | 62 |
| DGISTPKLKEDYQIG | 0.6 | 0.5 | 0.3 | 0.3 | 63 |
| GISTPKLKEDYQIGG | 0.4 | 0.5 | 0.4 | 0.4 | 64 |
| ISTPKLKEDYQIGGY | 1.0 | 0.7 | 0.5 | 0.7 | 65 |
| STPKLKEDYQIGGYS | 0.7 | 0.7 | 0.3 | 0.2 | 66 |
| TPKLKEDYQIGGYSE | 0.6 | 0.5 | 0.9 | 0.7 | 67 |
| PKLKEDYQIGGYSED | 0.4 | 0.4 | 0.7 | 0.5 | 68 |
| KLKEDYQIGGYSEDR | 0.5 | 0.5 | 0.6 | 0.5 | 69 |
| LKEDYQIGGYSEDRH | 0.5 | 0.5 | 0.5 | 0.4 | 70 |
| KEDYQIGGYSEDRHS | 0.5 | 0.5 | 0.4 | 0.3 | 71 |
| EDYQIGGYSEDRHSG | 0.4 | 0.4 | 0.5 | 0.4 | 72 |
| DYQIGGYSEDRHSGV | 0.5 | 0.6 | 0.7 | 0.5 | 73 |
| YQIGGYSEDRHSGVK | 0.4 | 0.3 | 0.3 | 0.3 | 74 |
| QIGGYSEDRHSGVKD | 0.5 | 0.3 | 0.4 | 0.3 | 75 |
| IGGYSEDRHSGVKDY | 0.5 | 0.4 | 0.6 | 0.7 | 76 |
| GGYSEDRHSGVKDYV | 0.4 | 0.6 | 0.5 | 0.4 | 77 |
| GYSEDRHSGVKDYVV | 0.8 | 0.8 | 0.6 | 0.6 | 78 |
| YSEDRHSGVKDYVVV | 0.6 | 0.7 | 0.8 | 0.7 | 79 |
| SEDRHSGVKDYVVVH | 0.8 | 1.0 | 0.5 | 0.5 | 80 |
| EDRHSGVKDYVVVHG | 0.9 | 0.9 | 0.7 | 1.1 | 81 |
| DRHSGVKDYVVVHGY | 0.7 | 0.7 | 0.4 | 0.6 | 82 |
| RHSGVKDYVVVHGYF | 0.5 | 0.6 | 0.6 | 0.7 | 83 |
| HSGVKDYVVVHGYFT | 0.8 | 0.7 | 2.3 | 2.2 | 84 |
| SGVKDYVVVHGYFTE | 0.6 | 0.5 | 0.5 | 0.2 | 85 |
| GVKDYVVVHGYFTEV | 0.7 | 0.5 | 1.3 | 1.1 | 86 |
| VKDYVVVHGYFTEVY | 0.5 | 0.5 | 0.6 | 0.5 | 709 |
| KDYVVVHGYFTEVYY | 0.6 | 0.5 | 0.5 | 0.5 | 710 |
| DYVVVHGYFTEVYYQ | 0.7 | 0.5 | 0.7 | 0.5 | 711 |
| YVVVHGYFTEVYYQL | 1.0 | 0.4 | 0.9 | 0.5 | 712 |
| VVVHGYFTEVYYQLE | 1.1 | 0.5 | 0.7 | 0.4 | 713 |
| VVHGYFTEVYYQLES | 0.8 | 0.4 | 0.6 | 0.4 | 714 |
| VHGYFTEVYYQLEST | 0.5 | 0.4 | 0.7 | 0.5 | 715 |
| HGYFTEVYYQLESTQ | 0.3 | 0.4 | 0.5 | 0.5 | 716 |
| GYFTEVYYQLESTQI | 0.4 | 0.4 | 0.7 | 0.6 | 717 |
| YFTEVYYQLESTQIT | 0.4 | 0.4 | 0.5 | 0.4 | 718 |
| FTEVYYQLESTQITT | 0.5 | 0.6 | 0.6 | 0.6 | 719 |
| TEVYYQLESTQITTD | 0.5 | 0.5 | 0.4 | 0.4 | 720 |
| EVYYQLESTQITTDT | 0.4 | 0.4 | 0.4 | 0.5 | 721 |
| VYYQLESTQITTDTG | 0.4 | 0.4 | 0.3 | 0.3 | 722 |
| YYQLESTQITTDTGI | 0.5 | 0.4 | 0.4 | 0.4 | 723 |
| YQLESTQITTDTGIE | 0.4 | 0.4 | 0.4 | 0.3 | 724 |
| QLESTQITTDTGIEN | 0.5 | 0.3 | 0.5 | 0.5 | 725 |
| LESTQITTDTGIENA | 0.4 | 0.3 | 0.4 | 0.4 | 726 |
| ESTQITTDTGIENAT | 0.5 | 0.4 | 0.4 | 0.4 | 727 |
| STQITTDTGIENATF | 0.6 | 0.5 | 0.6 | 0.5 | 728 |
| TQITTDTGIENATFF | 0.8 | 0.6 | 0.5 | 0.5 | 729 |
| QITTDTGIENATFFI | 0.6 | 0.5 | 0.8 | 0.5 | 730 |
| ITTDTGIENATFFIF | 0.9 | 0.4 | 0.9 | 0.6 | 731 |
| TTDTGIENATFFIFN | 1.0 | 0.6 | 0.8 | 0.8 | 732 |
| TDTGIENATFFIFNK | 0.4 | 0.6 | 0.6 | 0.8 | 733 |
| DTGIENATFFIFNKL | 0.5 | 0.6 | 0.9 | 0.7 | 734 |
| TGIENATFFIFNKLV | 0.7 | 0.8 | 0.7 | 0.8 | 735 |
| GIENATFFIFNKLVK | 0.5 | 0.6 | 0.5 | 0.5 | 736 |
| IENATFFIFNKLVKD | 0.3 | 0.4 | 0.5 | 0.5 | 737 |
| ENATFFIFNKLVKDP | 0.4 | 0.5 | 0.4 | 0.7 | 738 |
| NATFFIFNKLVKDPP | 0.4 | 0.4 | 0.4 | 0.3 | 739 |
| ATFFIFNKLVKDPPN | 0.5 | 0.5 | 0.8 | 0.8 | 87 |
| TFFIFNKLVKDPPNV | 0.6 | 0.7 | 0.6 | 0.6 | 88 |
| FFIFNKLVKDPPNVQ | 0.7 | 0.6 | 0.6 | 0.5 | 89 |
| FIFNKLVKDPPNVQI | 0.8 | 0.8 | 0.7 | 0.8 | 90 |
| IFNKLVKDPPNVQIH | 1.0 | 1.1 | 0.6 | 0.6 | 91 |
| FNKLVKDPPNVQIHT | 0.9 | 0.8 | 0.7 | 0.8 | 92 |
| NKLVKDPPNVQIHTI | 0.9 | 1.0 | 0.9 | 0.8 | 93 |
| KLVKDPPNVQIHTID | 0.5 | 0.4 | 0.4 | 0.4 | 94 |
| LVKDPPNVQIHTIDG | 0.4 | 0.4 | 0.7 | 0.6 | 95 |
| VKDPPNVQIHTIDGS | 0.4 | 0.4 | 0.6 | 0.5 | 96 |
| KDPPNVQIHTIDGSS | 0.3 | 0.3 | 1.1 | 1.1 | 97 |
| DPPNVQIHTIDGSSG | 0.3 | 0.3 | 0.5 | 0.4 | 740 |
| PPNVQIHTIDGSSGV | 0.5 | 0.5 | 0.7 | 0.5 | 741 |
| PNVQIHTIDGSSGVA | 0.6 | 0.5 | 0.5 | 0.5 | 742 |
| NVQIHTIDGSSGVAN | 0.5 | 0.5 | 0.4 | 0.4 | 743 |
| VQIHTIDGSSGVANP | 0.5 | 0.5 | 0.4 | 0.3 | 744 |
| QIHTIDGSSGVANPA | 0.6 | 0.6 | 0.4 | 0.2 | 745 |
| IHTIDGSSGVANPAM | 0.8 | 0.7 | 0.4 | 0.4 | 746 |
| HTIDGSSGVANPAMD | 0.5 | 0.4 | 0.2 | 0.3 | 747 |
| TIDGSSGVANPAMDP | 0.6 | 0.4 | 0.4 | 0.4 | 748 |
| IDGSSGVANPAMDPI | 0.6 | 0.6 | 0.7 | 0.6 | 749 |
| DGSSGVANPAMDPIY | 0.8 | 0.9 | 0.4 | 0.5 | 98 |
| GSSGVANPAMDPIYD | 0.7 | 0.6 | 0.4 | 0.4 | 99 |
| SSGVANPAMDPIYDE | 0.5 | 0.5 | 0.7 | 0.7 | 100 |
| SGVANPAMDPIYDEP | 0.3 | 0.4 | 0.5 | 0.5 | 101 |
| GVANPAMDPIYDEPT | 0.3 | 0.4 | 0.6 | 0.5 | 102 |
| VANPAMDPIYDEPTT | 0.2 | 0.4 | 0.5 | 0.5 | 103 |
| ANPAMDPIYDEPTTT | 0.3 | 0.4 | 0.4 | 0.4 | 104 |
| NPAMDPIYDEPTTTT | 0.3 | 0.4 | 0.4 | 0.4 | 105 |
| PAMDPIYDEPTTTTS | 0.4 | 0.4 | 0.4 | 0.5 | 106 |
| AMDPIYDEPTTTTSV | 0.6 | 0.8 | 0.7 | 0.7 | 107 |
| MDPIYDEPTTTTSVP | 0.5 | 0.6 | 0.3 | 0.3 | 108 |
| DPIYDEPTTTTSVPL | 0.5 | 0.5 | 0.7 | 0.8 | 109 |

TABLE 18
Binding of two control sera to linear and looped/cyclic
peptides of the protein X2 of SARS-CoV Urbani.

| | Control | Control serum | Control serum | Control Serum | |
|---|---|---|---|---|---|
| | serum LUMC | Blood-bank | LUMC | Blood-Bank | |
| Peptide | linear | linear | looped | Looped | SEQ |
| sequence | peptides | peptides | peptides | peptides | ID NO |
| 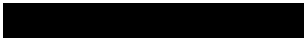 | 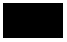 | 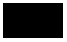 | 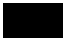 | 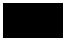 | 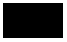 |
| 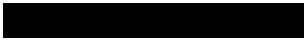 | 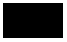 | 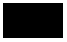 | 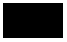 | 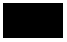 | 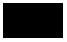 |
| 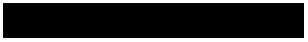 | 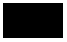 | 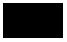 | 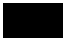 | 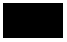 | 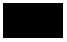 |
| 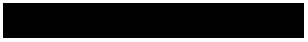 | 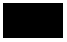 | 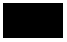 | 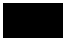 | 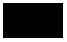 | 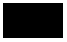 |
| 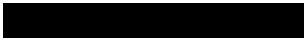 | 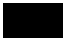 | 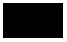 | 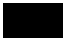 | 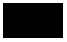 | 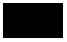 |
| 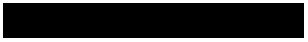 | 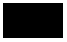 | 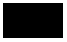 | 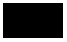 | 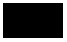 | 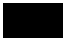 |
| 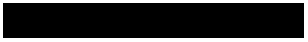 | 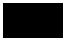 | 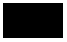 | 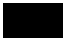 | 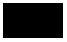 | 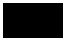 |
| 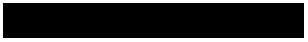 | 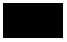 | 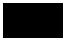 | 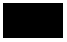 | 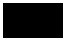 | 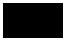 |
| 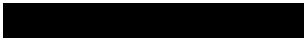 | 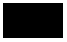 | 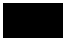 | 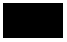 | 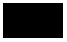 | 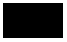 |
| THITMTTVYHITVSQ | 0.8 | 0.3 | 0.7 | 0.6 | 750 |
| HITMTTVYHITVSQI | 0.5 | 0.5 | 0.6 | 0.7 | 751 |
| ITMTTVYHITVSQIQ | 0.4 | 0.4 | 0.5 | 0.5 | 752 |
| TMTTVYHITVSQIQL | 0.8 | 0.6 | 0.5 | 0.6 | 753 |
| MTTVYHITVSQIQLS | 0.6 | 0.6 | 0.5 | 0.6 | 754 |
| TTVYHTTVSQIQLSL | 0.7 | 0.6 | 0.5 | 0.6 | 755 |
| TVYHITVSQIQLSLL | 0.5 | 0.5 | 0.5 | 0.6 | 756 |
| VYHITVSQIQLSLLK | 0.7 | 0.5 | 0.4 | 0.6 | 757 |
| YHITVSQIQLSLLKV | 0.7 | 0.6 | 0.4 | 0.3 | 758 |
| HITVSQIQLSLLKVT | 0.8 | 0.6 | 0.5 | 0.6 | 759 |
| ITVSQIQLSLLKVTA | 0.6 | 0.5 | 0.7 | 0.6 | 760 |
| TVSQIQLSLLKVTAF | 0.6 | 0.5 | 0.7 | 0.7 | 761 |
| VSQIQLSLLKVTAFQ | 0.8 | 0.5 | 0.6 | 0.6 | 762 |
| SQIQLSLLKVTAFQH | 0.7 | 0.5 | 0.6 | 0.6 | 763 |
| QIQLSLLKVTAFQHQ | 0.7 | 0.4 | 0.6 | 0.6 | 764 |
| IQLSLLKVTAFQHQN | 0.7 | 0.4 | 0.6 | 0.6 | 765 |
| QLSLLKVTAFQHQNS | 0.6 | 0.4 | 0.6 | 0.5 | 766 |
| LSLLKVTAFQHQNSK | 0.4 | 0.1 | 0.3 | 0.4 | 767 |
| SLLKVTAFQHQNSKK | 0.0 | 0.2 | 0.3 | 0.4 | 768 |
| LLKVTAFQHQNSKKT | 0.3 | 0.3 | 0.5 | 0.4 | 769 |
| LKVTAFQHQNSKKTT | 0.8 | 0.4 | 0.4 | 0.3 | 770 |
| KVTAFQHQNSKKTTK | 0.3 | 0.2 | 0.3 | 0.3 | 771 |
| VTAFQHQNSKKTTKL | 0.5 | 0.3 | 0.5 | 0.5 | 772 |
| TAFQHQNSKKTTKLV | 0.9 | 0.6 | 0.4 | 0.4 | 511 |
| AFQHQNSKKTTKLVV | 0.7 | 0.6 | 0.6 | 0.7 | 512 |
| FQHQNSKKTTKLVVI | 0.7 | 0.5 | 0.5 | 0.4 | 119 |
| QHQNSKKTTKLVVIL | 0.6 | 0.5 | 0.5 | 0.6 | 120 |
| HQNSKKTTKLVVILR | 0.8 | 0.5 | 0.6 | 0.6 | 121 |
| QNSKKTTKLVVILRI | 0.8 | 0.5 | 0.7 | 0.7 | 122 |
| NSKKTTKLVVILRIG | 0.9 | 0.4 | 0.7 | 0.7 | 123 |
| SKKTTKLVVILRIGT | 0.8 | 0.6 | 0.7 | 0.7 | 124 |
| KKTTKLVVILRIGTQ | 0.5 | 0.4 | 0.8 | 0.6 | 125 |
| KTTKLVVILRIGTQV | 0.5 | 0.4 | 0.7 | 0.7 | 126 |
| TTKLVVILRIGTQVL | 0.3 | 0.3 | 0.7 | 0.6 | 127 |
| TKLVVILRIGTQVLK | 0.5 | 0.7 | 0.7 | 0.6 | 128 |
| KLVVILRIGTQVLKT | 0.4 | 0.8 | 0.6 | 0.7 | 129 |
| LVVILRIGTQVLKTM | 0.7 | 0.7 | 0.6 | 0.8 | 773 |
| VVILRIGTQVLKTMS | 0.5 | 0.7 | 0.3 | 0.3 | 774 |
| VILRIGTQVLKTMSL | 0.5 | 0.8 | 0.7 | 0.7 | 775 |
| ILRIGTQVLKTMSLY | 0.4 | 0.6 | 0.7 | 0.7 | 776 |
| LRIGTQVLKTMSLYM | 0.4 | 0.6 | 0.7 | 0.7 | 130 |
| RIGTQVLKTMSLYMA | 0.5 | 0.8 | 0.5 | 0.6 | 131 |
| IGTQVLKTMSLYMAI | 0.5 | 0.8 | 1.0 | 0.9 | 132 |
| GTQVLKTMSLYMAIS | 0.6 | 0.6 | 0.6 | 0.7 | 133 |
| TQVLKTMSLYMAISP | 0.7 | 0.7 | 0.8 | 0.7 | 134 |
| QVLKTMSLYMAISPK | 0.7 | 0.9 | 0.4 | 0.4 | 135 |
| VLKTMSLYMAISPKF | 0.5 | 0.9 | 0.6 | 0.7 | 136 |
| LKTMSLYMAISPKFT | 0.6 | 0.7 | 0.6 | 0.6 | 137 |
| KTMSLYMAISPKFTT | 0.6 | 0.7 | 1.0 | 0.7 | 138 |
| TMSLYMAISPKFTTS | 0.7 | 0.5 | 0.8 | 0.8 | 777 |
| MSLYMAISPKFTTSL | 0.6 | 0.7 | 0.9 | 0.8 | 778 |
| SLYMAISPKFTTSLS | 0.6 | 0.7 | 0.9 | 0.6 | 779 |
| LYMAISPKFTTSLSL | 0.3 | 0.6 | 0.8 | 0.7 | 780 |
| YMAISPKFTTSLSLH | 0.4 | 0.7 | 0.7 | 0.7 | 781 |
| MAISPKFTTSLSLHK | 0.7 | 1.0 | 0.6 | 0.7 | 782 |
| AISPKFTTSLSLHKL | 0.6 | 0.8 | 0.6 | 0.8 | 783 |
| ISPKFTTSLSLHKLL | 0.3 | 0.7 | 0.5 | 0.7 | 784 |
| SPKFTTSLSLHKLLQ | 0.3 | 0.5 | 0.6 | 0.5 | 785 |
| PKFTTSLSLHKLLQT | 0.4 | 0.7 | 0.4 | 0.7 | 786 |
| KFTTSLSLHKLLQTL | 0.4 | 0.6 | 0.5 | 0.5 | 787 |
| FTTSLSLHKLLQTLV | 0.5 | 0.6 | 1.6 | 2.0 | 788 |
| TTSLSLHKLLQTLVL | 0.4 | 0.6 | 0.6 | 0.6 | 789 |
| TSLSLHKLLQTLVLK | 0.6 | 0.9 | 0.6 | 0.6 | 790 |
| SLSLHKLLQTLVLKM | 0.3 | 0.4 | 0.7 | 0.6 | 791 |
| LSLHKLLQTLVLKML | 0.2 | 0.5 | 0.7 | 0.6 | 792 |
| SLHKLLQTLVLKMLH | 0.3 | 0.6 | 0.7 | 0.7 | 793 |
| LHKLLQTLVLKMLHS | 0.3 | 0.5 | 0.7 | 0.8 | 794 |
| HKLLQTLVLKMLHSS | 0.4 | 0.6 | 0.7 | 0.7 | 795 |
| KLLQTLVLKMLHSSS | 0.3 | 0.7 | 0.6 | 0.7 | 796 |
| LLQTLVLKMLHSSSL | 0.3 | 0.5 | 0.6 | 0.6 | 797 |
| LQTLVLKMLHSSSLT | 0.5 | 0.7 | 0.5 | 0.5 | 798 |
| QTLVLKMLHSSSLTS | 0.4 | 0.7 | 0.6 | 0.5 | 799 |
| TLVLKMLHSSSLTSL | 0.5 | 0.9 | 0.7 | 0.9 | 800 |
| LVLKMLHSSSLTSLL | 0.2 | 0.5 | 0.6 | 0.7 | 801 |
| VLKMLHSSSLTSLLK | 0.4 | 0.7 | 0.3 | 0.5 | 802 |
| LKMLHSSSLTSLLKT | 0.4 | 0.7 | 0.5 | 0.6 | 803 |
| KMLHSSSLTSLLKTH | 0.5 | 0.7 | 0.4 | 0.3 | 804 |
| MLHSSSLTSLLKTHR | 0.4 | 0.5 | 0.5 | 0.6 | 805 |
| LHSSSLTSLLKTHRM | 0.4 | 0.7 | 0.4 | 0.4 | 806 |
| HSSSLTSLLKTHRMC | 0.4 | 0.8 | 0.5 | 0.5 | 807 |
| SSSLTSLLKTHRMCK | 0.7 | 1.0 | 0.4 | 0.4 | 808 |
| SSLTSLLKTHRMCKY | 0.4 | 0.8 | 0.6 | 0.7 | 809 |
| SLTSLLKTHRMCKYT | 0.4 | 0.8 | 0.5 | 0.4 | 810 |
| LTSLLKTHRMCKYTQ | 0.4 | 0.5 | 0.5 | 0.4 | 811 |
| TSLLKTHRMCKYTQS | 0.9 | 1.0 | 0.4 | 0.4 | 812 |
| SLLKTHRMCKYTQST | 0.8 | 0.9 | 0.4 | 0.4 | 813 |
| LLKTHRMCKYTQSTA | 0.6 | 0.7 | 0.4 | 0.4 | 814 |
| LKTHRMCKYTQSTAL | 0.6 | 0.7 | 0.6 | 0.7 | 815 |
| KTHRMCKYTQSTALQ | 0.5 | 0.6 | 0.4 | 0.5 | 816 |
| THRMCKYTQSTALQE | 0.7 | 0.9 | 0.4 | 0.5 | 817 |
| HRMCKYTQSTALQEL | 0.7 | 0.9 | 0.6 | 0.8 | 818 |
| RMCKYTQSTALQELL | 0.7 | 0.8 | 0.6 | 0.7 | 819 |
| MCKYTQSTALQELLI | 0.7 | 0.9 | 0.7 | 0.8 | 820 |
| CKYTQSTALQELLIQ | 0.5 | 0.6 | 0.6 | 0.4 | 821 |
| KYTQSTALQELLIQQ | 0.5 | 0.6 | 1.0 | 0.9 | 822 |
| YTQSTALQELLIQQW | 0.4 | 0.5 | 0.6 | 0.6 | 823 |
| TQSTALQELLIQQWI | 0.6 | 0.8 | 0.6 | 0.7 | 824 |
| QSTALQELLIQQWIQ | 0.4 | 0.6 | 0.6 | 0.6 | 825 |
| STALQELLIQQWIQF | 0.3 | 0.6 | 0.7 | 0.6 | 826 |
| TALQELLIQQWIQFM | 0.4 | 0.7 | 0.7 | 0.7 | 827 |
| ALQELLIQQWIQFMM | 0.4 | 0.5 | 0.7 | 0.7 | 828 |
| LQELLIQQWIQFMMS | 0.3 | 0.5 | 0.6 | 0.6 | 829 |
| QELLIQQWIQFMMSR | 0.3 | 0.5 | 0.5 | 0.7 | 830 |
| ELLIQQWIQFMMSRR | 0.4 | 0.5 | 0.5 | 0.6 | 831 |
| LLIQQWTQFMMSRRR | 0.4 | 0.5 | 0.5 | 0.6 | 832 |
| LIQQWIQFMMSRRRL | 0.5 | 0.7 | 0.6 | 0.4 | 833 |
| IQQWIQFMMSRRRLL | 0.5 | 0.7 | 0.6 | 0.5 | 834 |
| QQWIQFMMSRRRLLA | 0.6 | 0.8 | 0.5 | 0.7 | 835 |
| QWIQFMMSRRRLLAC | 0.5 | 0.8 | 0.4 | 0.3 | 836 |
| WIQFMMSRRRLLACL | 0.4 | 0.6 | 0.3 | 0.3 | 837 |
| IQFMMSRRRLLACLC | 0.6 | 0.8 | 0.4 | 0.3 | 838 |
| QFMMSRRRLLACLCK | 0.5 | 0.7 | 0.4 | 0.3 | 839 |
| FMMSRRRLLACLCKH | 0.4 | 0.7 | 0.5 | 0.6 | 840 |
| MMSRRRLLACLCKHK | 0.5 | 0.8 | 0.2 | 0.2 | 139 |
| MSRRRLLACLCKHKK | 0.5 | 0.7 | 0.2 | 0.3 | 140 |
| SRRRLLACLCKHKKV | 0.6 | 0.9 | 0.2 | 0.2 | 141 |
| RRRLLACLCKHKKVS | 0.6 | 0.7 | 0.2 | 0.3 | 142 |
| RRLLACLCKHKKVST | 0.7 | 0.9 | 0.3 | 0.2 | 143 |
| RLLACLCKHKKVSTN | 0.7 | 0.9 | 0.4 | 0.3 | 144 |
| LLACLCKHKKVSTNL | 0.8 | 0.8 | 0.7 | 0.5 | 145 |
| LACLCKHKKVSTNLC | 0.7 | 0.8 | 0.4 | 0.3 | 146 |
| ACLCKHKKVSTNLCT | 0.8 | 0.9 | 0.3 | 0.2 | 147 |
| CLCKHKKVSTNLCTH | 0.9 | 1.0 | 0.3 | 0.4 | 148 |
| LCKHKKVSTNLCTHS | 0.7 | 0.8 | 0.4 | 0.3 | 149 |
| CKHKKVSTNLCTHSF | 1.0 | 0.8 | 0.4 | 0.1 | 150 |
| KHKKVSTNLCTHSFR | 0.6 | 0.9 | 0.7 | 0.5 | 151 |
| HKKVSTNLCTHSFRK | 0.9 | 0.8 | 0.8 | 0.5 | 152 |
| KKVSTNLCTHSFRKK | 0.4 | 0.7 | 0.8 | 0.5 | 153 |
| KVSTNLCTHSFRKKQ | 0.8 | 1.0 | 0.7 | 0.4 | 154 |
| VSTNLCTHSFRKKQV | 0.6 | 0.8 | 0.7 | 0.5 | 155 |
| STNLCTHSFRKKQVR | 0.8 | 0.9 | 0.8 | 0.5 | 156 |

TABLE 19

Binding of two control sera to linear and
looped/cyclic peptides of the protein E of
SARS-CoV Urbani.

| | | | Con- | | |
|---|---|---|---|---|---|
| | | Control | trol | Control | |
| | Control | serum | serum | Serum | |
| | serum | Blood- | LUMC | Blood- | |
| | LUMC | bank | looped | Bank | SEQ |
| Peptide | linear | linear | pep- | Looped | ID |
| sequence | peptides | peptides | tides | peptides | NO |
| MYSFVSEETGTLIVN | 0.7 | 0.8 | 0.8 | 0.6 | 841 |
| YSFVSEETGTLIVNS | 1.0 | 0.7 | 0.8 | 0.6 | 842 |
| SFVSEETGTLIVNSV | 0.6 | 0.9 | 0.7 | 0.7 | 843 |
| VSEETGTLIVNSVLL | 0.5 | 0.8 | 0.7 | 0.9 | 844 |
| FVSEETGTLIVNSVL | 1.1 | 0.7 | 0.7 | 0.8 | 845 |
| SEETGTLIVNSVLLF | 0.6 | 0.5 | 0.5 | 0.6 | 846 |
| EETGTLIVNSVLLFL | 0.6 | 0.7 | 0.4 | 0.6 | 847 |
| ETGTLIVNSVLLFLA | 0.8 | 0.5 | 0.5 | 0.5 | 848 |
| TGTLIVNSVLLFLAF | 0.3 | 0.6 | 0.5 | 0.4 | 849 |
| GTLIVNSVLLFLAFV | 0.5 | 0.7 | 0.3 | 0.7 | 850 |
| TLIVNSVLLFLAFVV | 0.4 | 0.6 | 0.8 | 0.8 | 851 |
| LIVNSVLLFLAFVVF | 0.3 | 0.5 | 0.8 | 0.8 | 852 |
| IVNSVLLFLAFVVFL | 0.4 | 0.6 | 0.7 | 0.5 | 853 |
| VNSVLLFLAFVVFLL | 0.4 | 0.5 | 0.7 | 0.5 | 854 |
| NSVLLFLAFVVFLLV | 0.7 | 0.7 | 0.8 | 0.6 | 855 |
| SVLLFLAFVVFLLVT | 0.5 | 0.7 | 0.7 | 0.6 | 856 |
| VLLFLAFVVFLLVTL | 0.5 | 0.8 | 0.7 | 0.5 | 857 |
| LLFLAFVVFLLVTLA | 0.4 | 0.6 | 0.7 | 0.5 | 858 |
| LFLAFVVFLLVTLAI | 0.5 | 0.7 | 0.7 | 0.6 | 859 |
| FLAFVVFLLVTLAIL | 0.4 | 0.4 | 0.6 | 0.8 | 860 |
| LAFVVFLLVTLAILT | 0.5 | 0.6 | 0.6 | 0.8 | 861 |
| AFVVFLLVTLAILTA | 0.4 | 0.5 | 0.5 | 0.8 | 862 |
| FVVFLLVTLAILTAL | 0.5 | 0.5 | 0.6 | 0.7 | 863 |
| VVFLLVTLAILTALR | 0.4 | 0.6 | 0.6 | 0.6 | 864 |
| VFLLVTLAILTALRL | 0.3 | 0.5 | 0.5 | 0.4 | 865 |
| FLLVTLAILTALRLC | 0.5 | 0.6 | 0.1 | 0.5 | 866 |
| LLVTLAILTALRLCA | 0.3 | 0.7 | 0.8 | 0.8 | 867 |
| LVTLAILTALRLCAY | 0.3 | 0.6 | 0.8 | 0.6 | 868 |
| VTLAILTALRLCAYC | 0.6 | 0.5 | 0.8 | 0.9 | 869 |
| TLAILTALRLCAYCC | 0.4 | 0.6 | 0.8 | 0.7 | 870 |
| LAILTALRLCAYCCN | 0.6 | 0.7 | 0.8 | 0.7 | 871 |
| AILTALRLCAYCCNI | 0.6 | 0.6 | 0.8 | 0.6 | 872 |
| ILTALRLCAYCCNIV | 0.4 | 0.8 | 0.8 | 0.8 | 873 |
| LTALRLCAYCCNIVN | 0.5 | 0.7 | 0.6 | 0.7 | 874 |
| TALRLCAYCCNIVNV | 0.6 | 0.5 | 0.7 | 0.7 | 875 |
| ALRLCAYCCNIVNVS | 0.5 | 0.8 | 0.8 | 1.1 | 876 |
| LRLCAYCCNIVNVSL | 0.4 | 0.8 | 0.6 | 0.6 | 877 |
| RLCAYCCNIVNVSLV | 0.6 | 0.8 | 0.8 | 0.7 | 878 |
| LCAYCCNIVNVSLVK | 0.8 | 1.1 | 0.7 | 0.8 | 157 |
| CAYCCNIVNVSLVKP | 0.7 | 1.1 | 0.7 | 0.9 | 158 |
| AYCCNIVNVSLVKPT | 0.9 | 0.9 | 0.8 | 0.9 | 159 |
| YCCNIVNVSLVKPTV | 0.6 | 0.9 | 0.6 | 0.7 | 160 |
| CCNIVNVSLVKPTVY | 0.5 | 0.7 | 0.5 | 0.4 | 161 |
| CNIVNVSLVKPTVYV | 0.8 | 0.7 | 0.8 | 0.6 | 162 |
| NIVNVSLVKPTVYVY | 0.4 | 0.6 | 0.8 | 0.6 | 163 |
| IVNVSLVKPTVYVYS | 0.4 | 0.7 | 0.9 | 0.9 | 164 |
| VNVSLVKPTVYVYSR | 0.8 | 0.6 | 0.8 | 0.6 | 165 |
| NVSLVKPTVYVYSRV | 0.5 | 0.6 | 0.8 | 0.7 | 166 |
| VSLVKPTVYVYSRVK | 0.6 | 0.9 | 0.9 | 0.6 | 167 |
| SLVKPTVYVYSRVKN | 1.0 | 0.7 | 0.8 | 0.7 | 168 |
| LVKPTVYVYSRVKNL | 0.4 | 0.8 | 0.7 | 0.7 | 169 |
| VKPTVYVYSRVKNLN | 0.4 | 0.8 | 0.8 | 0.8 | 170 |
| KPTVYVYSRVKNLNS | 0.8 | 0.7 | 0.9 | 0.9 | 171 |
| PTVYVYSRVKNLNSS | 0.5 | 0.8 | 0.8 | 1.1 | 172 |
| TVYVYSRVKNLNSSE | 0.3 | 0.5 | 0.9 | 0.7 | 173 |
| VYVYSRVKNLNSSEG | 0.8 | 0.5 | 0.8 | 0.8 | 174 |
| YVYSRVKNLNSSEGV | 0.6 | 0.8 | 0.8 | 0.9 | 175 |
| VYSRVKNLNSSEGVP | 0.6 | 0.9 | 0.7 | 1.0 | 176 |
| YSRVKNLNSSEGVPD | 0.8 | 0.6 | 0.8 | 0.4 | 177 |
| SRVKNLNSSEGVPDL | 0.7 | 0.9 | 0.7 | 0.5 | 178 |
| RVKNLNSSEGVPDLL | 1.0 | 1.0 | 1.0 | 0.7 | 179 |
| VKNLNSSEGVPDLLV | 1.1 | 0.7 | 0.9 | 0.8 | 180 |

TABLE 20
Binding of two control sera to linear and looped/cyclic
peptides of the protein M of SARS-CoV Urbani.

| | Control serum | Control serum | Control serum | Control Serum | |
|---|---|---|---|---|---|
| | LUMC | Blood-bank | LUMC | Blood-Bank | |
| Peptide | linear | linear | looped | Looped | SEQ |
| sequence | peptides | peptides | peptides | peptides | ID NO |
| MADNGTITVEELKQL | 0.6 | 0.5 | 0.6 | 0.4 | 181 |
| ADNGTITVEELKQLL | 0.5 | 0.7 | 0.7 | 0.5 | 182 |
| DNGTITVEELKQLLE | 0.8 | 0.7 | 0.7 | 0.4 | 183 |
| NGTITVEELKQLLEQ | 0.5 | 0.7 | 0.8 | 0.6 | 184 |
| 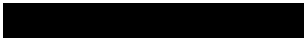 | 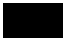 | 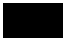 | 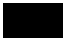 | 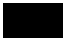 | 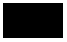 |
| 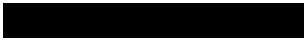 | 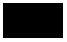 | 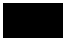 | 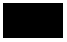 | 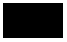 | 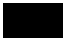 |
| 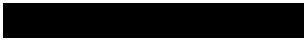 | 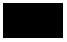 | 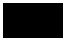 | 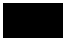 | 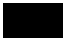 | 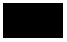 |
| 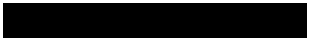 | 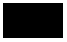 | 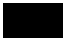 | 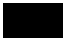 | 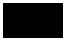 | 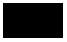 |
| 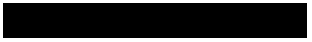 | 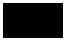 | 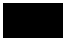 | 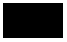 | 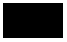 | 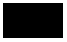 |
| 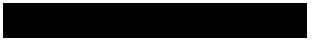 | 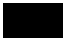 | 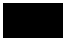 | 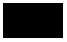 | 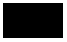 | 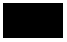 |
| ELKQLLEQWNLVIGF | 0.7 | 0.8 | 0.7 | 0.6 | 879 |
| LKQLLEQWNLVIGFL | 0.3 | 0.7 | 0.7 | 0.7 | 880 |
| KQLLEQWNLVIGFLF | 0.6 | 0.5 | 0.7 | 0.6 | 881 |
| QLLEQWNLVIGFLFL | 0.3 | 0.5 | 0.7 | 0.5 | 882 |
| LLEQWNLVIGFLFLA | 0.4 | 0.5 | 0.5 | 0.2 | 883 |
| LEQWNLVIGFLFLAW | 0.3 | 0.6 | 0.5 | 0.6 | 884 |
| EQWNLVIGFLFLAWI | 0.4 | 0.5 | 0.7 | 0.6 | 885 |
| QWNLVIGFLFLAWIM | 0.3 | 0.7 | 0.6 | 0.6 | 886 |
| WNLVIGFLFLAWIML | 0.6 | 0.6 | 0.7 | 0.5 | 887 |
| NLVIGFLFLAWIMLL | 0.3 | 0.5 | 0.8 | 0.6 | 888 |
| LVIGFLFLAWIMLLQ | 0.5 | 0.6 | 0.7 | 0.6 | 889 |
| VIGFLFLAWIMLLQF | 0.3 | 0.5 | 0.7 | 0.7 | 890 |
| IGFLFLAWIMLLQFA | 0.7 | 0.7 | 0.8 | 0.7 | 891 |
| GFLFLAWIMLLQFAY | 0.3 | 0.7 | 0.7 | 0.6 | 892 |
| FLFLAWIMLLQFAYS | 0.5 | 0.5 | 0.7 | 0.8 | 893 |
| LFLAWIMLLQFAYSN | 0.2 | 0.5 | 0.6 | 0.7 | 894 |
| FLAWIMLLQFAYSNR | 0.5 | 0.5 | 0.7 | 0.8 | 895 |
| LAWIMLLQFAYSNRN | 0.2 | 0.6 | 0.6 | 0.6 | 896 |
| AWIMLLQFAYSNRNR | 0.5 | 0.7 | 0.7 | 0.7 | 897 |
| WIMLLQFAYSNRNRF | 0.3 | 0.7 | 0.7 | 0.7 | 898 |
| IMLLQFAYSNRNRFL | 0.6 | 0.5 | 0.6 | 0.5 | 899 |
| MLLQFAYSNRNRFLY | 0.3 | 0.6 | 0.5 | 0.4 | 900 |
| LLQFAYSNRNRFLYI | 0.6 | 0.6 | 0.5 | 0.5 | 901 |
| LQFAYSNRNRFLYII | 0.4 | 0.6 | 0.6 | 0.6 | 902 |
| QFAYSNRNRFLYIIK | 0.8 | 0.6 | 0.8 | 0.6 | 191 |
| FAYSNRNRFLYIIKL | 0.4 | 0.6 | 0.6 | 0.4 | 192 |
| AYSNRNRFLYIIKLV | 0.7 | 0.7 | 0.8 | 0.6 | 193 |
| YSNRNRFLYIIKLVF | 0.4 | 0.7 | 0.9 | 0.6 | 194 |
| SNRNRFLYIIKLVFL | 0.7 | 0.7 | 0.9 | 0.7 | 195 |
| NRNRFLYIIKLVFLW | 0.4 | 0.7 | 0.7 | 0.8 | 196 |
| RNRFLYIIKLVFLWL | 0.7 | 0.6 | 0.8 | 0.7 | 197 |
| NRFLYIIKLVFLWLL | 0.3 | 0.7 | 0.6 | 0.7 | 198 |
| RFLYIIKLVFLWLLW | 0.8 | 0.7 | 0.8 | 0.7 | 199 |
| FLYIIKLVFLWLLWP | 0.4 | 0.8 | 0.9 | 0.8 | 200 |
| LYIIKLVFLWLLWPV | 0.7 | 0.7 | 0.6 | 0.7 | 903 |
| YIIKLVFLWLLWPVT | 0.5 | 0.8 | 0.8 | 0.8 | 904 |
| IIKLVFLWLLWPVTL | 0.6 | 0.5 | 0.6 | 0.7 | 905 |
| IKLVFLWLLWPVTLA | 0.4 | 0.7 | 0.7 | 0.9 | 906 |
| KLVFLWLLWPVTLAC | 0.5 | 0.7 | 0.6 | 0.7 | 907 |
| LVFLWLLWPVTLACF | 0.2 | 0.5 | 0.7 | 0.7 | 908 |
| VFLWLLWPVTLACFV | 0.6 | 0.6 | 0.8 | 0.8 | 909 |
| FLWLLWPVTLACFVL | 0.2 | 0.4 | 0.7 | 0.5 | 910 |
| LWLLWPVTLACFVLA | 0.5 | 0.5 | 0.6 | 0.5 | 911 |
| WLLWPVTLACFVLAA | 0.2 | 0.4 | 0.6 | 0.5 | 912 |
| LLWPVTLACFVLAAV | 0.7 | 0.7 | 0.7 | 0.6 | 913 |
| LWPVTLACFVLAAVY | 0.4 | 0.6 | 0.7 | 0.6 | 914 |
| WPVTLACFVLAAVYR | 0.5 | 0.7 | 1.2 | 0.8 | 915 |
| PVTLACFVLAAVYRI | 0.2 | 0.5 | 0.7 | 0.6 | 916 |
| VTLACFVLAAVYRIN | 0.5 | 0.5 | 0.7 | 0.8 | 917 |
| TLACFVLAAVYRINW | 0.2 | 0.5 | 0.7 | 0.8 | 918 |
| LACPVLAAVYRINWV | 0.6 | 0.7 | 0.7 | 0.7 | 919 |
| ACFVLAAVYRINWVT | 0.3 | 0.7 | 0.7 | 0.7 | 920 |
| CFVLAAVYRINWVTG | 0.6 | 0.7 | 0.8 | 0.6 | 921 |
| FVLAAVYRINWVTGG | 0.5 | 0.8 | 0.8 | 0.6 | 922 |
| VLAAVYRINWVTGGI | 1.0 | 0.9 | 0.8 | 0.8 | 923 |
| LAAVYRINWVTGGIA | 0.5 | 0.6 | 0.6 | 0.4 | 924 |
| AAVYRINWVTGGIAI | 1.0 | 0.8 | 0.5 | 0.8 | 925 |
| AVYRINWVTGGIAIA | 0.5 | 0.7 | 0.8 | 0.8 | 926 |
| VYRINWVTGGIAIAM | 1.0 | 0.7 | 0.8 | 0.7 | 927 |
| YRINWVTGGIAIAMA | 0.6 | 0.8 | 0.6 | 0.7 | 928 |
| RINWVTGGIAIAMAC | 0.8 | 0.8 | 0.8 | 0.5 | 929 |
| INWVTGGIAIAMACI | 0.5 | 0.8 | 0.9 | 0.6 | 201 |
| NWVTGGIAIAMACIV | 0.8 | 0.7 | 0.9 | 0.6 | 202 |
| WVTGGIAIAMACIVG | 0.4 | 0.8 | 0.9 | 0.8 | 203 |
| VTGGIAIAMACIVGL | 1.3 | 1.1 | 0.8 | 0.7 | 204 |
| TGGIAIAMACIVGLM | 0.6 | 1.1 | 1.1 | 1.1 | 205 |
| GGIAIAMACIVGLMW | 0.8 | 0.8 | 0.9 | 0.8 | 206 |
| GIAIAMACIVGLMWL | 0.5 | 0.8 | 0.8 | 0.7 | 207 |
| IAIAMACIVGLMWLS | 0.6 | 0.5 | 0.7 | 0.8 | 208 |
| AIAMACIVGLMWLSY | 0.3 | 0.7 | 0.6 | 0.6 | 930 |
| IAMACIVGLMWLSYF | 0.6 | 0.5 | 0.7 | 0.6 | 931 |
| AMACIVGLMWLSYFV | 0.3 | 0.6 | 0.7 | 0.6 | 932 |
| MACIVGLMWLSYFVA | 0.5 | 0.5 | 0.6 | 0.4 | 933 |
| ACIVGLMWLSYFVAS | 0.3 | 0.6 | 0.5 | 0.6 | 934 |
| CIVGLMWLSYFVASF | 0.5 | 0.4 | 0.7 | 0.6 | 935 |
| IVGLMWLSYFVASFR | 0.2 | 0.5 | 0.6 | 0.7 | 936 |
| VGLMWLSYFVASFRL | 0.5 | 0.6 | 0.6 | 0.6 | 937 |
| GLMWLSYFVASFRLF | 0.2 | 0.6 | 0.6 | 0.7 | 938 |
| LMWLSYFVASFRLFA | 0.4 | 0.5 | 0.6 | 0.6 | 209 |
| MWLSYFVASFRLFAR | 0.2 | 0.5 | 0.7 | 0.7 | 210 |
| WLSYFVASFRLFART | 0.6 | 0.7 | 0.8 | 0.6 | 211 |
| LSYFVASFRLFARTR | 0.3 | 0.6 | 1.0 | 0.8 | 212 |
| SYFVASFRLFARTRS | 0.5 | 0.6 | 0.9 | 1.0 | 213 |
| YFVASFRLFARTRSM | 0.3 | 0.6 | 0.7 | 0.9 | 214 |
| FVASFRLFARTRSMW | 1.0 | 0.9 | 0.7 | 0.6 | 215 |
| VASFRLFARTRSMWS | 0.4 | 0.7 | 0.6 | 0.8 | 216 |
| ASFRLFARTRSMWSF | 0.6 | 0.7 | 0.7 | 0.7 | 939 |
| SFRLFARTRSMWSFN | 0.4 | 0.6 | 0.6 | 0.7 | 940 |
| FRLFARTRSMWSFNP | 0.8 | 0.8 | 0.6 | 0.8 | 941 |
| RLFARTRSMWSFNPE | 0.5 | 0.8 | 0.5 | 0.7 | 942 |
| LFARTRSMWSFNPET | 0.8 | 0.7 | 0.6 | 0.6 | 943 |
| FARTRSMWSFNPETN | 0.5 | 0.9 | 0.6 | 0.8 | 944 |
| ARTRSMWSFNPETNI | 0.9 | 0.7 | 0.7 | 0.6 | 945 |
| RTRSMWSFNPETNIL | 0.7 | 0.9 | 0.7 | 0.7 | 946 |
| TRSMWSFNPETNILL | 0.8 | 0.7 | 0.9 | 0.9 | 947 |
| RSMWSFNPETNILLN | 0.5 | 0.8 | 0.7 | 0.7 | 948 |
| SMWSFNPETNILLNV | 0.8 | 0.8 | 0.7 | 0.8 | 949 |
| MWSFNPETNILLNVP | 0.5 | 0.9 | 0.8 | 0.8 | 950 |
| WSFNPETNILLNVPL | 1.5 | 1.3 | 0.8 | 0.6 | 951 |
| SFNPETNILLNVPLR | 0.4 | 0.7 | 1.0 | 1.0 | 952 |
| FNPETNILLNVPLRG | 0.7 | 0.6 | 0.9 | 0.8 | 953 |
| NPETNILLNVPLRGT | 0.4 | 0.7 | 0.9 | 0.8 | 954 |
| PETNILLNVPLRGTI | 0.8 | 0.8 | 0.7 | 0.8 | 955 |
| ETNILLNVPLRGTIV | 0.3 | 0.7 | 0.9 | 1.0 | 956 |
| TNILLNVPLRGTIVT | 0.7 | 0.8 | 0.6 | 0.8 | 957 |
| NILLNVPLRGTIVTR | 0.3 | 0.8 | 0.7 | 0.8 | 217 |
| ILLNVPLRGTIVTRP | 0.4 | 0.6 | 0.5 | 0.5 | 218 |
| LLNVPLRGTIVTRPL | 0.4 | 0.7 | 0.5 | 0.9 | 219 |
| LNVPLRGTIVTRPLM | 0.5 | 0.7 | 0.7 | 0.6 | 220 |
| NVPLRGTIVTRPLME | 0.4 | 0.6 | 0.9 | 0.7 | 221 |
| VPLRGTIVTRPLMES | 0.6 | 0.9 | 0.7 | 0.6 | 222 |
| PLRGTIVTRPLMESE | 0.3 | 0.6 | 0.8 | 0.5 | 223 |
| LRGTIVTRPLMESEL | 0.5 | 0.5 | 0.7 | 0.7 | 224 |
| RGTIVTRPLMESELV | 0.4 | 0.7 | 0.8 | 0.6 | 225 |
| GTIVTRPLMESELVI | 0.6 | 0.7 | 1.0 | 0.8 | 226 |
| TIVTRPLMESELVIG | 0.5 | 0.8 | 1.0 | 0.9 | 227 |
| IVTRPLMESELVIGA | 0.8 | 0.7 | 1.0 | 0.9 | 229 |
| VTRPLMESELVIGAV | 0.4 | 0.6 | 1.0 | 0.9 | 230 |
| TRPLMESELVIGAVI | 1.0 | 0.9 | 1.0 | 1.1 | 231 |
| RPLMESELVIGAVII | 0.5 | 0.6 | 0.8 | 0.8 | 232 |
| PLMESELVIGAVIIR | 0.8 | 1.0 | 0.8 | 0.9 | 958 |
| LMESELVIGAVIIRG | 0.5 | 0.8 | 0.6 | 0.7 | 959 |
| MESELVIGAVIIRGH | 0.8 | 0.8 | 0.6 | 0.7 | 960 |
| ESELVIGAVIIRGHL | 0.4 | 0.6 | 0.5 | 0.7 | 961 |
| SELVIGAVIIRGHLR | 0.9 | 0.8 | 0.6 | 0.6 | 962 |
| ELVIGAVIIRGHLRM | 0.5 | 0.7 | 0.6 | 0.7 | 963 |
| LVIGAVIIRGHLRMA | 0.7 | 0.6 | 0.7 | 0.7 | 964 |
| 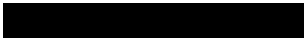 | 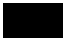 | 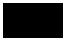 | 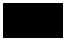 | 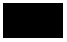 | 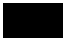 |
| 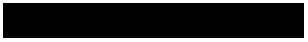 | 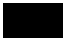 | 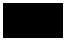 | 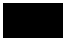 | 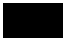 | 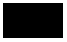 |
| 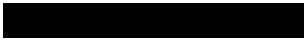 | 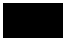 | 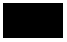 | 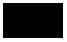 | 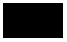 | 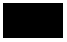 |
| 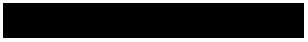 | 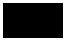 | 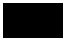 | 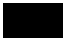 | 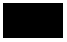 | 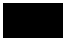 |
| 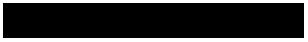 | 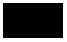 | 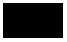 | 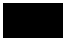 | 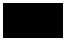 | 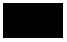 |
| 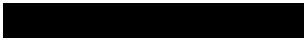 | 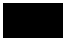 | 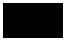 | 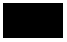 | 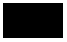 | 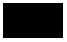 |
| 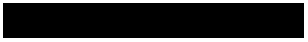 | 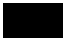 | 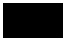 | 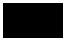 | 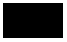 | 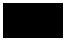 |
| 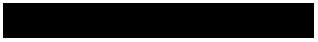 | 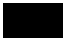 | 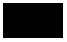 | 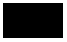 | 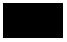 | 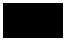 |
| 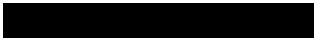 | 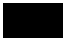 | 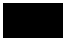 | 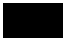 | 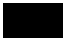 | 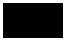 |
| 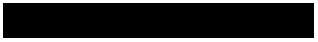 | 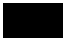 | 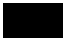 | 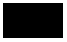 | 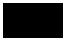 | 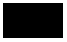 |
| LRMAGHPLGRCDIKD | 0.5 | 0.7 | 0.8 | 0.8 | 243 |
| RMAGHPLGRCDIKDL | 0.7 | 0.7 | 0.8 | 0.9 | 244 |
| MAGHPLGRCDIKDLP | 0.5 | 0.7 | 0.5 | 0.5 | 245 |
| AGHPLGRCDIKDLPK | 0.8 | 1.1 | 0.7 | 0.9 | 246 |
| GHPLGRCDIKDLPKE | 0.6 | 0.6 | 0.7 | 0.7 | 247 |
| HPLGRCDIKDLPKEI | 0.7 | 1.0 | 0.7 | 1.0 | 248 |
| PLGRCDIKDLPKEIT | 0.4 | 0.6 | 0.8 | 0.9 | 249 |
| LGRCDIKDLPKEITV | 0.5 | 0.7 | 0.8 | 0.8 | 250 |
| GRCDIKDLPKEITVA | 0.1 | 0.5 | 0.9 | 1.0 | 251 |
| RCDIKDLPKEITVAT | 0.5 | 0.7 | 0.7 | 0.6 | 965 |
| CDIKDLPKEITVATS | 0.2 | 0.4 | 0.7 | 0.6 | 966 |
| DIKDLPKEITVATSR | 0.5 | 0.6 | 0.9 | 0.8 | 967 |
| IKDLPKEITVATSRT | 0.4 | 0.6 | 0.7 | 0.5 | 968 |
| KDLPKEITVATSRTL | 0.6 | 0.7 | 0.8 | 0.6 | 969 |
| DLPKEITVATSRTLS | 0.2 | 0.5 | 0.7 | 0.7 | 970 |
| LPKEITVATSRTLSY | 0.7 | 0.8 | 0.6 | 0.6 | 971 |
| PKEITVATSRTLSYY | 0.3 | 0.5 | 0.6 | 0.6 | 972 |
| KEITVATSRTLSYYK | 0.6 | 0.8 | 0.7 | 0.7 | 973 |
| EITVATSRTLSYYKL | 0.4 | 0.7 | 0.6 | 0.7 | 974 |
| ITVATSRTLSYYKLG | 0.7 | 0.8 | 0.6 | 0.5 | 975 |
| TVATSRTLSYYKLGA | 0.6 | 0.8 | 0.7 | 0.7 | 976 |
| VATSRTLSYYKLGAS | 0.6 | 0.8 | 0.6 | 0.5 | 977 |
| ATSRTLSYYKLGASQ | 0.3 | 0.7 | 0.6 | 0.6 | 978 |
| TSRTLSYYKLGASQR | 1.0 | 0.9 | 0.8 | 0.6 | 979 |
| SRTLSYYKLGASQRV | 0.5 | 0.9 | 0.8 | 0.6 | 980 |
| RTLSYYKLGASQPVG | 0.8 | 0.9 | 0.8 | 0.6 | 981 |
| TLSYYKLGASQRVGT | 0.5 | 0.8 | 0.8 | 0.8 | 252 |
| LSYYKLGASQRVGTD | 0.8 | 0.8 | 0.7 | 0.7 | 253 |
| SYYKLGASQRVGTDS | 0.4 | 0.8 | 0.7 | 0.8 | 254 |
| YYKLGASQRVGTDSG | 0.8 | 1.0 | 0.7 | 0.7 | 255 |
| YKLGASQRVGTDSGF | 0.4 | 0.7 | 0.9 | 0.7 | 256 |
| KLGASQRVGTDSGFA | 0.9 | 0.9 | 0.9 | 0.8 | 257 |
| LGASQRVGTDSGFAA | 0.5 | 0.9 | 0.8 | 0.6 | 258 |
| GASQRVGTDSGFAAY | 0.9 | 0.9 | 0.7 | 0.7 | 259 |
| ASQRVGTDSGFAAYN | 0.5 | 0.8 | 0.8 | 0.7 | 260 |
| SQRVGTDSGFAAYNR | 1.0 | 0.9 | 0.6 | 0.7 | 982 |
| QRVGTDSGFAAYNRY | 0.3 | 0.6 | 0.8 | 0.7 | 983 |
| RVGTDSGFAAYNRYR | 0.4 | 0.7 | 0.6 | 0.5 | 984 |
| VGTDSGFAAYNRYRI | 0.3 | 0.7 | 0.6 | 0.5 | 985 |
| GTDSGFAAYNRYRIG | 0.5 | 0.7 | 0.7 | 0.6 | 986 |
| TDSGFAAYNRYRIGN | 0.3 | 0.7 | 0.7 | 0.6 | 987 |
| DSGFAAYNRYRIGNY | 0.5 | 0.5 | 0.5 | 0.5 | 988 |
| SGFAAYNRYRIGNYK | 0.4 | 0.6 | 0.8 | 0.7 | 989 |
| GFAAYNRYRIGNYKL | 0.8 | 0.7 | 0.6 | 0.5 | 990 |
| FAAYNRYRIGNYKLN | 0.3 | 0.7 | 0.7 | 0.6 | 991 |
| AAYNRYRIGNYKLNT | 0.8 | 0.9 | 0.7 | 0.8 | 992 |
| AYNRYRIGNYKLNTD | 0.3 | 0.6 | 0.7 | 0.6 | 993 |
| YNRYRIGNYKLNTDH | 0.6 | 0.5 | 0.9 | 0.8 | 994 |
| NRYRIGNYKLNTDHA | 0.2 | 0.2 | 0.7 | 0.7 | 995 |
| RYRIGNYKLNTDHAG | 0.5 | 0.7 | 0.7 | 0.7 | 996 |
| YRIGNYKLNTDHAGS | 0.3 | 0.6 | 0.6 | 0.6 | 997 |
| RIGNYKLNTDHAGSN | 0.6 | 0.9 | 0.8 | 0.8 | 998 |
| IGNYKLNTDHAGSND | 0.3 | 0.5 | 0.8 | 0.7 | 261 |
| GNYKLNTDHAGSNDN | 0.7 | 0.6 | 0.7 | 0.6 | 262 |
| NYKLNTDHAGSNDNI | 0.5 | 0.7 | 0.8 | 0.6 | 263 |
| YKLNTDHAGSNDNIA | 0.7 | 0.6 | 0.6 | 0.6 | 264 |
| KLNTDHAGSNDNIAL | 0.7 | 0.9 | 0.9 | 1.0 | 265 |
| LNTDHAGSNDNIALL | 0.7 | 0.6 | 0.9 | 0.7 | 266 |
| NTDHAGSNDNIALLV | 0.5 | 0.8 | 0.9 | 1.0 | 267 |
| TDHAGSNDNIALLVQ | 1.0 | 0.7 | 1.0 | 1.0 | 268 |

TABLE 21

Binding of two control sera to linear and
looped/cyclic peptides of the protein X3 of
SARS-CoV Urbani.

| | | | | | |
|---|---|---|---|---|---|
| | Con- | | Con- | Con- | |
| | trol | Control | trol | trol | |
| | serum | serum | serum | Serum | |
| | LUMC | Blood- | LUMC | Blood- | |
| | linear | bank | looped | Bank | SEQ |
| Peptide | pep- | linear | pep- | Looped | ID |
| sequence | tides | peptides | tides | peptides | NO |

| | | | | | |
|---|---|---|---|---|---|
| MFHLVDFQVTIAEIL | 0.9 | 0.9 | 1.0 | 0.8 | 999 |
| FHLVDFQVTIAEILI | 0.6 | 0.8 | 0.8 | 0.7 | 1000 |
| HLVDFQVTIAEILII | 0.7 | 0.8 | 0.7 | 0.9 | 1001 |
| LVDFQVTIAEILIII | 0.9 | 0.8 | 0.7 | 0.6 | 1002 |
| VDFQVTIAEILIIIM | 0.8 | 0.8 | 0.8 | 0.6 | 1003 |
| DFQVTIAEILIIIMR | 0.6 | 0.8 | 0.7 | 0.8 | 1004 |
| FQVTIAEILIIIMRT | 0.4 | 0.8 | 0.6 | 0.7 | 1005 |
| QVTIAEILIIIMRTF | 0.7 | 0.7 | 0.7 | 0.9 | 1006 |
| VTIAEILIIIMRTFR | 0.6 | 0.8 | 0.8 | 0.8 | 1007 |
| TIAEILIIIMRTFRI | 0.6 | 0.8 | 0.7 | 1.1 | 1008 |
| IAEILIIIMRTFRIA | 0.5 | 0.9 | 0.9 | 0.8 | 1009 |
| AEILIIIMRTFRIAI | 0.4 | 0.7 | 0.8 | 0.7 | 269 |
| EILIIIMRTFRIAIW | 0.5 | 0.8 | 0.6 | 0.6 | 270 |
| ILIIIMRTFRIAIWN | 0.5 | 0.8 | 0.3 | 0.7 | 271 |
| LIIIMRTFRIAIWNL | 0.6 | 0.7 | 0.7 | 0.6 | 272 |
| IIIMRTFRIAIWNLD | 0.9 | 0.9 | 0.8 | 0.8 | 273 |
| IIMRTFRIAIWNLDV | 0.7 | 0.9 | 0.6 | 0.9 | 274 |
| IMRTFRIAIWNLDVI | 0.8 | 0.8 | 0.8 | 0.9 | 275 |
| MRTFRIAIWNLDVII | 0.6 | 0.7 | 0.8 | 0.8 | 276 |
| RTFRIAIWNLDVIIS | 0.6 | 0.7 | 0.8 | 0.9 | 277 |
| TFRIAIWNLDVIISS | 0.7 | 0.8 | 0.6 | 0.8 | 1010 |
| FRIAIWNLDVIISSI | 0.6 | 0.7 | 0.7 | 0.8 | 1011 |
| RIAIWNLDVIISSIV | 0.6 | 0.7 | 0.8 | 0.9 | 1012 |
| IAIWNLDVIISSIVR | 0.5 | 0.6 | 0.7 | 0.7 | 1013 |
| AIWNLDVIISSIVRQ | 0.7 | 0.7 | 0.7 | 0.8 | 1014 |
| IWNLDVIISSIVRQL | 0.5 | 0.6 | 0.7 | 0.7 | 1015 |
| WNLDVIISSIVRQLF | 0.3 | 0.6 | 0.7 | 0.8 | 1016 |
| NLDVIISSIVRQLFK | 0.6 | 0.8 | 0.8 | 0.8 | 1017 |
| LDVIISSIVRQLFKP | 0.4 | 0.6 | 0.8 | 0.7 | 1018 |
| DVIISSIVRQLFKPL | 0.4 | 0.6 | 0.5 | 0.5 | 1019 |
| VIISSIVRQLFKPLT | 0.7 | 0.8 | 0.8 | 0.7 | 278 |
| IISSIVRQLFKPLTK | 0.8 | 0.8 | 0.6 | 0.6 | 279 |
| ISSIVRQLFKPLTKK | 0.9 | 0.8 | 0.7 | 0.8 | 280 |
| SSIVRQLFKPLTKKN | 1.1 | 1.2 | 0.7 | 0.8 | 281 |
| SIVRQLFKPLTKKNY | 0.7 | 0.8 | 0.8 | 0.8 | 282 |
| IVRQLFKPLTKKNYS | 0.8 | 0.9 | 0.9 | 0.9 | 283 |
| VRQLFKPLTKKNYSE | 0.7 | 0.9 | 0.7 | 0.9 | 284 |
| RQLFKPLTKKNYSEL | 0.8 | 0.9 | 0.7 | 0.9 | 285 |
| QLFKPLTKKNYSELD | 0.8 | 0.8 | 0.8 | 0.8 | 286 |
| LFKPLTKKNYSELDD | 0.6 | 0.7 | 0.9 | 0.8 | 287 |
| FKPLTKKNYSELDDE | 0.8 | 0.8 | 0.8 | 0.8 | 288 |
| KPLTKKNYSELDDEE | 0.8 | 0.7 | 0.7 | 0.8 | 289 |
| PLTKKNYSELDDEEP | 0.9 | 0.8 | 0.8 | 0.8 | 290 |
| LTKKNYSELDDEEPM | 0.6 | 0.9 | 1.0 | 0.8 | 291 |
| TKKNYSELDDEEPME | 0.4 | 0.8 | 0.9 | 0.4 | 292 |
| KKNYSELDDEEPMEL | 0.4 | 0.5 | 0.9 | 0.6 | 293 |
| KNYSELDDEEPMELD | 0.6 | 0.7 | 0.8 | 0.6 | 294 |
| NYSELDDEEPMELDY | 0.9 | 0.9 | 0.9 | 0.8 | 295 |
| YSELDDEEPMELDYP | 0.9 | 0.7 | 0.8 | 0.8 | 296 |

TABLE 22
Binding of two control sera to linear and looped/cyclic
peptides of the protein X4 of SARS-CoV Urbani.

| | Control serum | Control serum | Control serum | Control Serum | |
|---|---|---|---|---|---|
| | LUMC | Blood-bank | LUMC | Blood-Bank | |
| Peptide | linear | linear | looped | Looped | SEQ |
| sequence | peptides | peptides | peptides | peptides | ID NO |
| MKIILFLTLIVFTSC | 0.5 | 0.6 | 0.7 | 0.6 | 1020 |
| KIILFLTLIVFTSCE | 0.9 | 0.8 | 0.9 | 0.8 | 1021 |
| IILFLTLIVFTSCEL | 0.9 | 0.7 | 0.6 | 0.7 | 1022 |
| ILFLTLIVFTSCELY | 0.7 | 0.6 | 0.8 | 0.7 | 1023 |
| LFLTLIVFTSCELYH | 0.7 | 0.7 | 0.9 | 0.9 | 1024 |
| FLTLIVFTSCELYHY | 0.7 | 0.6 | 0.7 | 0.6 | 1025 |
| LTLIVFTSCELYHYQ | 0.7 | 0.7 | 0.8 | 0.7 | 1026 |
| TLIVFTSCELYHYQE | 0.8 | 0.7 | 1.0 | 0.7 | 1027 |
| LIVFTSCELYHYQEC | 0.7 | 0.6 | 0.8 | 0.7 | 1028 |
| IVFTSCELYHYQECV | 0.8 | 0.8 | 0.9 | 0.8 | 1029 |
| VFTSCELYHYQECVR | 0.5 | 0.7 | 0.9 | 0.7 | 1030 |
| FTSCELYHYQECVRG | 0.7 | 0.7 | 0.8 | 0.8 | 1031 |
| TSCELYHYQECVRGT | 0.6 | 0.6 | 0.8 | 0.8 | 1032 |
| SCELYHYQECVRGTT | 0.3 | 0.5 | 0.8 | 0.6 | 1033 |
| CELYHYQECVRGTTV | 0.5 | 0.5 | 0.6 | 0.5 | 1034 |
| ELYHYQECVRGTTVL | 0.6 | 0.7 | 0.7 | 0.7 | 297 |
| LYHYQECVRGTTVLL | 0.6 | 0.6 | 0.6 | 0.7 | 298 |
| YHYQECVRGTTVLLK | 0.7 | 0.8 | 0.8 | 0.7 | 299 |
| HYQECVRGTTVLLKE | 0.7 | 0.8 | 1.0 | 0.9 | 300 |
| YQECVRGTTVLLKEP | 0.7 | 0.8 | 0.8 | 0.9 | 301 |
| QECVRGTTVLLKEPC | 1.0 | 0.9 | 0.8 | 0.9 | 302 |
| ECVRGTTVLLKEPCP | 0.7 | 0.9 | 0.8 | 0.9 | 303 |
| CVRGTTVLLKEPCPS | 0.7 | 0.8 | 0.9 | 0.9 | 304 |
| VRGTTVLLKEPCPSG | 1.0 | 0.8 | 0.9 | 0.8 | 305 |
| RGTTVLLKEPCPSGT | 0.8 | 0.9 | 0.8 | 0.9 | 306 |
| GTTVLLKEPCPSGTY | 0.7 | 0.7 | 0.8 | 0.7 | 307 |
| TTVLLKEPCPSGTYE | 0.9 | 0.9 | 1.0 | 0.9 | 308 |
| TVLLKEPCPSGTYEG | 0.8 | 0.7 | 0.8 | 0.7 | 309 |
| VLLKEPCPSGTYEGN | 0.7 | 0.8 | 0.8 | 0.9 | 1035 |
| LLKEPCPSGTYEGNS | 0.6 | 0.6 | 0.8 | 0.6 | 1036 |
| LKEPCPSGTYEGNSP | 0.3 | 0.6 | 0.7 | 0.5 | 1037 |
| KEPCPSGTYEGNSPF | 0.6 | 0.6 | 0.8 | 0.5 | 1038 |
| EPCPSGTYEGNSPFH | 0.5 | 0.6 | 0.8 | 0.6 | 1039 |
| PCPSGTYEGNSPFHP | 0.5 | 0.6 | 0.9 | 0.7 | 1040 |
| CPSGTYEGNSPFHPL | 0.6 | 0.7 | 0.8 | 0.8 | 310 |
| PSGTYEGNSPFHPLA | 0.7 | 0.9 | 0.9 | 0.8 | 311 |
| SGTYEGNSPFHPLAD | 0.8 | 0.8 | 0.8 | 0.8 | 312 |
| GTYEGNSPFHPLADN | 0.8 | 0.7 | 0.7 | 0.9 | 313 |
| 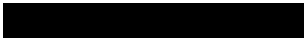 | 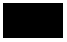 | 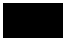 | 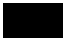 | 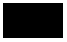 | 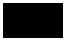 |
| 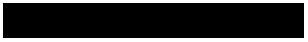 | 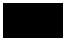 | 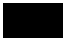 | 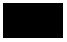 | 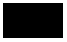 | 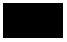 |
| 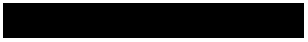 | 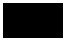 | 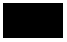 | 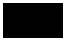 | 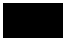 | 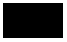 |
| 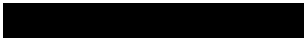 | 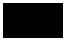 | 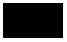 | 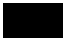 | 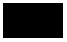 | 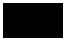 |
| 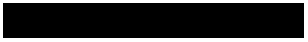 | 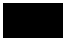 | 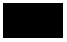 | 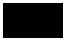 | 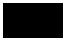 | 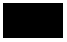 |
| 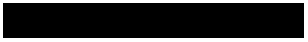 | 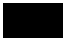 | 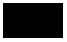 | 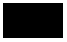 | 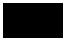 | 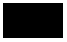 |
| PFHPLADNKFALTCT | 0.8 | 0.7 | 0.9 | 0.9 | 320 |
| FHPLADNKFALTCTS | 0.8 | 0.8 | 0.8 | 0.8 | 321 |
| HPLADNKFALTCTST | 0.6 | 0.7 | 0.9 | 0.8 | 322 |
| PLADNKFALTCTSTH | 0.5 | 0.8 | 0.7 | 0.6 | 323 |
| LADNKFALTCTSTHF | 0.7 | 0.7 | 0.7 | 0.7 | 324 |
| ADNKFALTCTSTHFA | 0.9 | 0.8 | 0.9 | 0.7 | 325 |
| DNKFALTCTSTHFAF | 0.6 | 0.7 | 0.8 | 0.6 | 326 |
| NKFALTCTSTHFAFA | 0.5 | 0.6 | 0.8 | 0.9 | 1041 |
| KFALTCTSTHFAFAC | 0.7 | 0.7 | 0.8 | 0.9 | 1042 |
| FALTCTSTHFAFACA | 0.6 | 0.6 | 0.6 | 0.7 | 1043 |
| ALTCTSTHFAFACAD | 0.8 | 0.7 | 0.8 | 0.9 | 1044 |
| LTCTSTHFAFACADG | 0.8 | 0.8 | 0.6 | 0.7 | 1045 |
| TCTSTHFAFACADGT | 0.9 | 0.8 | 0.8 | 0.7 | 1046 |
| CTSTHFAFACADGTR | 0.8 | 0.7 | 0.7 | 0.8 | 1047 |
| TSTHFAFACADGTRH | 0.8 | 0.7 | 0.8 | 0.8 | 1048 |
| STHFAFACADGTRHT | 0.5 | 0.6 | 0.9 | 0.7 | 1049 |
| THFAFACADGTRHTY | 0.7 | 0.7 | 0.7 | 0.7 | 1050 |
| HFAFACADGTRHTYQ | 0.7 | 0.6 | 0.7 | 0.8 | 1051 |
| FAFACADGTRHTYQL | 0.5 | 0.7 | 0.7 | 0.8 | 1052 |
| AFACADGTRHTYQLR | 0.5 | 0.6 | 0.8 | 0.7 | 1053 |
| FACADGTRHTYQLRA | 0.4 | 0.7 | 0.4 | 0.3 | 531 |
| ACADGTRHTYQLRAR | 0.6 | 0.6 | 0.5 | 0.5 | 532 |
| CADGTRHTYQLRARS | 0.5 | 0.7 | 0.6 | 0.5 | 533 |
| ADGTRHTYQLRARSV | 0.6 | 0.6 | 0.6 | 0.6 | 534 |
| DGTRHTYQLRARSVS | 0.5 | 0.6 | 0.7 | 0.8 | 535 |
| GTRHTYQLRARSVSP | 0.6 | 0.8 | 0.8 | 0.7 | 536 |
| TRHTYQLRARSVSPK | 0.9 | 0.9 | 0.7 | 0.7 | 537 |
| RHTYQLRARSVSPKL | 0.7 | 0.8 | 0.7 | 0.6 | 538 |
| HTYQLRARSVSPKLF | 0.9 | 1.0 | 0.7 | 0.8 | 539 |
| TYQLRARSVSPKLFI | 0.7 | 1.0 | 0.9 | 0.9 | 540 |
| YQLRARSVSPKLFIR | 0.6 | 0.6 | 0.7 | 0.7 | 541 |
| QLRARSVSPKLFIRQ | 0.6 | 0.7 | 0.8 | 0.9 | 542 |
| LRARSVSPKLFIRQE | 0.6 | 0.6 | 0.7 | 0.6 | 543 |
| RARSVSPKLFIRQEE | 0.5 | 0.6 | 0.8 | 0.7 | 544 |
| ARSVSPKLFIRQEEV | 0.6 | 0.7 | 0.7 | 0.7 | 1054 |
| RSVSPKLFIRQEEVQ | 0.4 | 0.5 | 0.7 | 0.6 | 1055 |
| SVSPKLFIRQEEVQQ | 0.3 | 0.6 | 0.7 | 0.4 | 1056 |
| VSPKLFIRQEEVQQE | 0.4 | 0.5 | 0.7 | 0.4 | 1057 |
| SPKLFIRQEEVQQEL | 0.5 | 0.5 | 0.8 | 0.6 | 1058 |
| PKLFIRQEEVQQELY | 0.5 | 0.7 | 0.7 | 0.6 | 1059 |
| KLFIRQEEVQQELYS | 0.5 | 0.5 | 0.8 | 0.8 | 1060 |
| LFIRQEEVQQELYSP | 0.7 | 0.7 | 0.8 | 0.8 | 1061 |
| FIRQEEVQQELYSPL | 0.7 | 0.7 | 0.9 | 0.9 | 327 |
| IRQEEVQQELYSPLF | 0.7 | 0.7 | 0.9 | 0.7 | 328 |
| RQEEVQQELYSPLFL | 0.6 | 0.7 | 0.9 | 0.8 | 329 |
| QEEVQQELYSPLFLI | 0.8 | 0.8 | 0.8 | 0.7 | 330 |
| EEVQQELYSPLFLIV | 0.6 | 0.6 | 1.0 | 1.0 | 331 |
| EVQQELYSPLFLIVA | 0.5 | 0.5 | 0.8 | 0.7 | 332 |
| VQQELYSPLFLIVAA | 0.5 | 0.6 | 0.6 | 0.7 | 333 |
| QQELYSPLFLIVAAL | 0.4 | 0.5 | 0.6 | 0.6 | 1062 |
| QELYSPLFLIVAALV | 0.6 | 0.6 | 0.7 | 0.7 | 1063 |
| ELYSPLFLIVAALVF | 0.3 | 0.5 | 0.7 | 0.7 | 1064 |
| LYSPLFLIVAALVFL | 0.4 | 0.5 | 0.6 | 0.6 | 1065 |
| YSPLFLIVAALVFLI | 0.5 | 0.6 | 0.7 | 0.4 | 1066 |
| SPLFLIVAALVFLIL | 0.3 | 0.4 | 0.5 | 0.3 | 1067 |
| PLFLIVAALVFLILC | 0.4 | 0.4 | 0.5 | 0.5 | 1068 |
| LFLIVAALVFLILCF | 0.4 | 0.5 | 0.4 | 0.7 | 1069 |
| FLIVAALVFLILCFT | 0.4 | 0.4 | 0.6 | 0.7 | 1070 |
| LIVAALVFLILCFTI | 0.4 | 0.6 | 0.5 | 0.5 | 1071 |
| IVAALVFLILCFTIK | 0.5 | 0.6 | 0.7 | 0.8 | 1072 |
| VAALVFLILCFTIKR | 0.4 | 0.5 | 0.6 | 0.8 | 1073 |
| AALVFLILCFTIKRK | 0.7 | 0.9 | 0.6 | 0.8 | 1074 |
| ALVFLILCFTIKRKT | 0.6 | 0.8 | 0.7 | 0.8 | 1075 |
| LVFLILCFTIKRKTE | 0.6 | 0.8 | 0.6 | 0.8 | 1076 |

TABLE 23
Binding of two control sera to linear and looped/cyclic
peptides of the protein X5 of SARS-CoV Urbani.

| | Control serum | Control serum | Control serum | Control Serum | |
|---|---|---|---|---|---|
| | LUMC | Blood-bank | LUMC | Blood-Bank | |
| Peptide | linear | linear | looped | Looped | SEQ |
| sequence | peptides | peptides | peptides | peptides | ID NO |
| MCLKILVRYNTRGNT | 0.6 | 0.8 | 0.5 | 0.4 | 1077 |
| CLKILVRYNTRGNTY | 0.4 | 0.8 | 0.6 | 0.4 | 1078 |
| LKILVRYNTRGNTYS | 0.5 | 0.7 | 0.6 | 0.4 | 1079 |
| KILVRYNTRGNTYST | 0.6 | 0.8 | 0.6 | 0.5 | 1080 |
| ILVRYNTRGNTYSTA | 0.5 | 0.7 | 0.6 | 0.4 | 1081 |
| LVRYNTRGNTYSTAW | 0.5 | 0.8 | 0.5 | 0.3 | 1082 |
| VRYNTRGNTYSTAWL | 0.5 | 0.9 | 0.5 | 0.3 | 1083 |
| RYNTRGNTYSTAWLC | 0.3 | 0.7 | 0.1 | 0.0 | 1084 |
| YNTRGNTYSTAWLCA | 0.6 | 0.7 | 0.5 | 0.5 | 1085 |
| NTRGNTYSTAWLCAL | 0.7 | 0.8 | 0.5 | 0.4 | 1086 |
| TRGNTYSTAWLCALG | 0.6 | 0.7 | 0.6 | 0.4 | 1087 |
| RGNTYSTAWLCALGK | 0.6 | 1.0 | 0.6 | 0.4 | 1088 |
| GNTYSTAWLCALGKV | 0.5 | 0.9 | 0.6 | 0.4 | 1089 |
| NTYSTAWLCALGKVL | 0.4 | 0.8 | 0.5 | 0.4 | 1090 |
| TYSTAWLCALGKVLP | 0.5 | 0.9 | 0.6 | 0.5 | 1091 |
| YSTAWLCALGKVLPF | 0.5 | 0.8 | 0.5 | 0.4 | 1092 |
| STAWLCALGKVLPFH | 0.6 | 0.9 | 0.6 | 0.5 | 1093 |
| TAWLCALGKVLPFHR | 0.4 | 0.7 | 0.6 | 0.4 | 1094 |
| AWLCALGKVLPFHRW | 0.6 | 0.9 | 0.5 | 0.4 | 1095 |
| WLCALGKVLPFHRWH | 0.6 | 0.9 | 0.6 | 0.4 | 1096 |
| LCALGKVLPFHRWHT | 0.5 | 0.7 | 0.6 | 0.4 | 1097 |
| CALGKVLPFHRWHTM | 0.7 | 0.8 | 0.6 | 0.4 | 1098 |
| ALGKVLPFHRWHTMV | 0.5 | 0.8 | 0.5 | 0.1 | 1099 |
| LGKVLPFHRWHTMVQ | 0.5 | 0.8 | 0.4 | 0.4 | 1100 |
| GKVLPFHRWHTMVQT | 0.3 | 0.6 | 0.5 | 0.5 | 1101 |
| KVLPFHRWHTMVQTC | 0.4 | 0.6 | 0.5 | 0.6 | 1102 |
| VLPFHRWHTMVQTCT | 0.5 | 0.6 | 0.0 | 0.6 | 1103 |
| LPFHRWHTMVQTCTP | 0.5 | 0.6 | 0.4 | 0.5 | 1104 |
| PFHRWHTMVQTCTPN | 0.5 | 0.7 | 0.4 | 0.5 | 1105 |
| FHRWHTMVQTCTPNV | 0.5 | 0.7 | 0.5 | 0.4 | 1106 |
| HRWHTMVQTGTPNVT | 0.4 | 0.8 | 0.4 | 0.4 | 1107 |
| RWHTMVQTCTPNVTI | 0.6 | 0.9 | 0.9 | 0.9 | 334 |
| WHTMVQTCTPNVTIN | 0.5 | 1.0 | 0.4 | 0.5 | 335 |
| HTMVQTCTPNVTINC | 0.7 | 0.9 | 0.6 | 0.8 | 336 |
| TMVQTCTPNVTINCQ | 0.7 | 0.7 | 0.7 | 0.9 | 337 |
| MVQTGTPNVTINCQD | 0.6 | 0.8 | 0.3 | 0.3 | 338 |
| VQTCTPNVTINCQDP | 0.5 | 0.6 | 0.4 | 0.6 | 1108 |
| QTCTPNVTINCQDPA | 0.4 | 0.6 | 0.3 | 0.4 | 1109 |
| TCTPNVTINCQDPAG | 0.5 | 0.7 | 0.2 | 0.4 | 1110 |
| CTPNVTINCQDPAGG | 0.3 | 0.7 | 0.4 | 0.4 | 1111 |
| TPNVTINCQDPAGGA | 0.5 | 0.6 | 0.2 | 0.3 | 1112 |
| PNVTINCQDPAGGAL | 0.7 | 1.0 | 0.4 | 0.4 | 339 |
| NVTINCQDPAGGALI | 0.6 | 0.8 | 0.7 | 0.7 | 340 |
| VTINCQDPAGGALIA | 0.6 | 0.8 | 0.5 | 0.5 | 341 |
| TINCQDPAGGALIAR | 0.6 | 1.0 | 1.0 | 1.3 | 342 |
| INCQDPAGGALIARC | 0.6 | 1.1 | 0.7 | 0.9 | 343 |
| NCQDPAGGALIARCW | 0.3 | 0.6 | 0.6 | 0.8 | 344 |
| CQDPAGGALIARCWY | 0.3 | 0.7 | 0.7 | 0.9 | 345 |
| QDPAGGALIARCWYL | 0.3 | 0.7 | 0.6 | 0.8 | 346 |
| DPAGGALIARCWYLH | 0.3 | 0.7 | 0.6 | 0.8 | 1113 |
| PAGGALIARCWYLHE | 0.4 | 0.7 | 0.5 | 0.8 | 1114 |
| AGGALIARCWYLHEG | 0.5 | 0.6 | 0.4 | 0.7 | 1115 |
| GGALIARCWYLHEGH | 0.4 | 0.5 | 0.6 | 0.6 | 1116 |
| GALIARCWYLHEGHQ | 0.4 | 0.6 | 0.2 | 0.3 | 1117 |
| ALIARCWYLHEGHQT | 0.4 | 0.6 | 0.1 | 0.4 | 1118 |
| LIARCWYLHEGHQTA | 0.4 | 0.6 | 0.0 | 0.3 | 1119 |
| 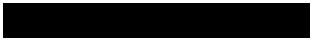 | 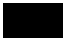 | 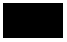 | 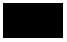 | 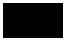 | 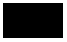 |
| 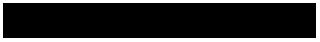 | 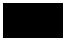 | 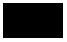 | 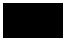 | 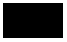 | 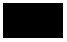 |
| 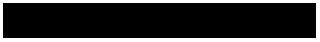 | 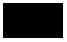 | 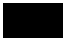 | 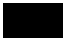 | 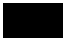 | 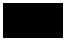 |
| 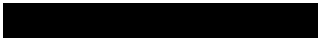 | 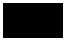 | 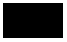 | 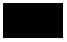 | 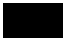 | 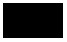 |
| 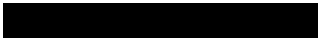 | 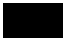 | 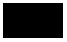 | 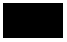 | 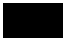 | 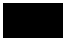 |
| 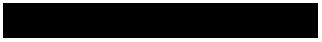 | 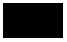 | 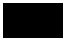 | 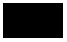 | 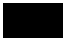 | 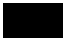 |
| 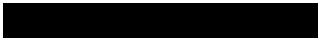 | 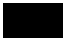 | 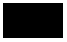 | 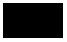 | 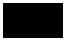 | 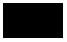 |
| 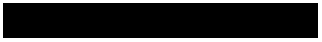 | 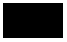 | 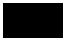 | 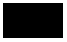 | 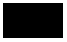 | 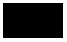 |
| EGHQTAAFRDVLVVL | 0.9 | 0.7 | 1.6 | 0.9 | 355 |
| GHQTAAFRDVLVVLN | 0.8 | 0.5 | 0.6 | 0.6 | 356 |
| HQTAAFRDVLVVLNK | 0.9 | 0.5 | 0.6 | 0.8 | 357 |
| QTAAFRDVLVVLNKR | 0.8 | 0.6 | 0.5 | 0.7 | 1120 |
| TAAFRDVLVVLNKRT | 0.8 | 0.7 | 0.6 | 0.8 | 1121 |
| AAFRDVLVVLNKRTN | 0.8 | 0.6 | 0.4 | 0.7 | 1122 |

TABLE 24
Binding of two control sera to linear and looped/cyclic
peptides of the protein N of SARS-CoV Urbani.

| | Control serum | Control serum | Control serum | Control Serum | |
|---|---|---|---|---|---|
| | LUMC | Blood-bank | LUMC | Blood-Bank | |
| Peptide | linear | linear | looped | Looped | SEQ |
| sequence | peptides | peptides | peptides | peptides | ID NO |
| MSDNGPQSNQRSAPR | 0.5 | 0.6 | 0.5 | 0.6 | 1123 |
| SDNGPQSNQRSAPRI | 0.5 | 0.6 | 0.5 | 0.5 | 1124 |
| DNGPQSNQRSAPRIT | 0.7 | 0.7 | 0.4 | 0.5 | 1125 |
| NGPQSNQRSAPRITF | 0.6 | 0.8 | 0.8 | 0.7 | 592 |
| GPQSNQRSAPRITFG | 0.5 | 0.6 | 0.8 | 0.7 | 593 |
| PQSNQRSAPRITFGG | 0.6 | 0.5 | 0.7 | 0.8 | 594 |
| QSNQRSAPRITFGGP | 0.5 | 0.6 | 0.9 | 0.7 | 595 |
| SNQRSAPRITFGGPT | 0.5 | 0.6 | 0.7 | 0.7 | 596 |
| NQRSAPRITFGGPTD | 0.5 | 0.6 | 0.7 | 0.6 | 597 |
| QRSAPRITFGGPTDS | 0.5 | 0.6 | 0.7 | 0.7 | 598 |
| RSAPRITFGGPTDST | 0.5 | 0.6 | 0.6 | 0.6 | 599 |
| SAPRITFGGPTDSTD | 0.5 | 0.4 | 0.4 | 0.5 | 600 |
| APRITFGGPTDSTDN | 0.5 | 0.6 | 0.6 | 0.6 | 601 |
| PRITFGGPTDSTDNN | 0.5 | 0.6 | 0.6 | 0.6 | 602 |
| RITFGGPTDSTDNNQ | 0.5 | 0.6 | 0.5 | 0.6 | 603 |
| ITFGGPTDSTDNNQN | 0.6 | 0.5 | 0.7 | 0.7 | 604 |
| TFGGPTDSTDNNQNG | 0.7 | 0.8 | 0.5 | 0.5 | 1126 |
| FGGPTDSTDNNQNGG | 0.5 | 0.6 | 0.4 | 0.4 | 1127 |
| GGPTDSTDNNQNGGR | 0.8 | 0.7 | 0.5 | 0.5 | 1128 |
| GPTDSTDNNQNGGRN | 0.7 | 0.8 | 0.5 | 0.6 | 1129 |
| PTDSTDNNQNGGRNG | 0.8 | 1.0 | 0.4 | 0.5 | 1130 |
| TDSTDNNQNGGRNGA | 0.8 | 1.1 | 0.8 | 0.7 | 1131 |
| DSTDNNQNGGRNGAR | 0.7 | 0.8 | 0.7 | 0.6 | 1132 |
| STDNNQNGGRNGARP | 0.6 | 0.7 | 0.5 | 0.5 | 1133 |
| TDNNQNGGRNGARPK | 0.8 | 0.9 | 0.5 | 0.5 | 1134 |
| DNNQNGGRNGARPKQ | 0.6 | 0.8 | 0.6 | 0.7 | 1135 |
| NNQNGGRNGARPKQR | 0.8 | 0.9 | 0.6 | 0.5 | 1136 |
| NQNGGRNGARPKQRR | 0.8 | 0.7 | 0.5 | 0.6 | 1137 |
| QNGGRNGARPKQRRP | 0.8 | 0.6 | 0.8 | 0.6 | 1138 |
| NGGRNGARPKQRRPQ | 0.6 | 0.6 | 0.7 | 0.7 | 1139 |
| GGRNGARPKQRRPQG | 0.6 | 0.7 | 0.5 | 0.5 | 1140 |
| GRNGARPKQRRPQGL | 0.6 | 0.7 | 0.5 | 0.5 | 1141 |
| RNGARPKQRRPQGLP | 0.6 | 0.8 | 0.6 | 0.5 | 1142 |
| NGARPKQRRPQGLPN | 0.6 | 0.7 | 0.6 | 0.6 | 1143 |
| GARPKQRRPQGLPNN | 0.7 | 0.7 | 0.6 | 0.5 | 1144 |
| ARPKQRRPQGLPNNT | 0.6 | 0.6 | 0.5 | 0.6 | 1145 |
| RPKQRRPQGLPNNTA | 0.7 | 0.8 | 0.5 | 0.4 | 1146 |
| PKQRRPQGLPNNTAS | 0.7 | 0.8 | 1.0 | 0.8 | 1147 |
| KQRRPQGLPNNTASW | 0.5 | 0.7 | 0.6 | 0.6 | 1148 |
| QRRPQGLPNNTASWF | 0.6 | 0.7 | 0.9 | 0.8 | 1149 |
| RRPQGLPNNTASWFT | 0.7 | 0.5 | 0.6 | 0.7 | 1150 |
| RPQGLPNNTASWFTA | 0.8 | 0.8 | 0.9 | 0.9 | 1151 |
| PQGLPNNTASWFTAL | 0.7 | 0.7 | 0.9 | 0.9 | 1152 |
| QGLPNNTASWFTALT | 0.7 | 0.6 | 0.8 | 0.8 | 1153 |
| GLPNNTASWFTALTQ | 0.6 | 0.7 | 0.7 | 0.8 | 1154 |
| LPNNTASWFTALTQH | 0.7 | 0.7 | 0.7 | 0.8 | 1155 |
| PNNTASWFTALTQHG | 0.7 | 0.5 | 0.7 | 0.7 | 1156 |
| NNTASWFTALTQHGK | 0.7 | 0.6 | 0.4 | 0.5 | 1157 |
| NTASWFTALTQHGKE | 0.6 | 0.5 | 0.4 | 0.5 | 1158 |
| TASWFTALTQHGKEE | 0.5 | 0.6 | 0.3 | 0.4 | 1159 |
| ASWFTALTQHGKEEL | 0.5 | 0.7 | 0.4 | 0.5 | 1160 |
| SWFTALTQHGKEELR | 0.7 | 0.6 | 0.3 | 0.4 | 1161 |
| WFTALTQHGKEELRF | 0.7 | 0.8 | 0.5 | 0.6 | 1162 |
| FTALTQHGKEELRFP | 0.7 | 0.7 | 0.3 | 0.5 | 1163 |
| TALTQHGKEELRFPR | 0.6 | 0.8 | 0.8 | 0.8 | 1164 |
| ALTQHGKEELRFPRG | 0.7 | 0.9 | 0.4 | 0.4 | 1165 |
| LTQHGKEELRFPRGQ | 0.6 | 0.8 | 0.6 | 0.6 | 1166 |
| TQHGKEELRFPRGQG | 0.8 | 0.9 | 0.6 | 0.6 | 1167 |
| QHGKEELRFPRGQGV | 0.7 | 0.8 | 0.7 | 0.7 | 1168 |
| HGKEELRFPRGQGVP | 0.6 | 0.8 | 0.5 | 0.5 | 1169 |
| GKEELRFPRGQGVPI | 0.8 | 0.9 | 0.9 | 0.9 | 1170 |
| KEELRFPRGQGVPIN | 0.7 | 0.8 | 0.7 | 0.7 | 1171 |
| EELRFPRGQGVPINT | 0.8 | 0.8 | 1.1 | 1.3 | 1172 |
| ELRFPRGQGVPINTN | 0.8 | 0.6 | 0.7 | 0.7 | 1173 |
| LRFPRGQGVPINTNS | 0.7 | 0.7 | 0.6 | 0.6 | 1174 |
| RFPRGQGVPINTNSG | 0.6 | 0.8 | 0.5 | 0.6 | 1175 |
| FPRGQGVPINTNSGP | 0.7 | 0.8 | 0.5 | 0.6 | 1176 |
| PRGQGVPINTNSGPD | 0.5 | 0.7 | 0.3 | 0.4 | 1177 |
| RGQGVPINTNSGPDD | 0.5 | 0.5 | 0.4 | 0.4 | 1178 |
| GQGVPINTNSGPDDQ | 0.5 | 0.5 | 0.3 | 0.5 | 1179 |
| QGVPINTNSGPDDQI | 0.6 | 0.6 | 0.8 | 1.1 | 1180 |
| GVPINTNSGPDDQIG | 0.7 | 0.7 | 0.4 | 0.4 | 1181 |
| VPINTNSGPDDQIGY | 0.8 | 0.7 | 0.7 | 0.8 | 1182 |
| PINTNSGPDDQIGYY | 0.7 | 0.7 | 0.6 | 0.5 | 1183 |
| INTNSGPDDQIGYYR | 0.7 | 0.8 | 0.7 | 0.6 | 1184 |
| NTNSGPDDQIGYYRR | 0.7 | 0.7 | 0.9 | 0.7 | 1185 |
| TNSGPDDQIGYYRRA | 0.9 | 1.2 | 0.9 | 0.7 | 1186 |
| NSGPDDQIGYYRRAT | 0.7 | 0.7 | 0.8 | 0.7 | 1187 |
| SGPDDQIGYYRRATR | 0.7 | 0.8 | 0.9 | 0.8 | 545 |
| GPDDQIGYYRRATRR | 0.8 | 0.8 | 0.9 | 0.9 | 546 |
| PDDQIGYYRRATRRV | 0.6 | 0.6 | 0.8 | 0.8 | 547 |
| DDQIGYYRRATRRVR | 1.0 | 1.0 | 0.8 | 0.8 | 548 |
| DQIGYYRRATRRVRG | 0.7 | 0.8 | 0.7 | 0.8 | 549 |
| QIGYYRRATRRVRGG | 0.6 | 0.8 | 0.7 | 0.8 | 550 |
| IGYYRRATRRVRGGD | 0.5 | 0.7 | 0.7 | 0.7 | 551 |
| GYYRRATRRVRGGDG | 0.6 | 0.6 | 0.5 | 0.6 | 552 |
| YYRRATRRVRGGDGK | 0.6 | 0.6 | 0.2 | 0.3 | 1188 |
| YRRATRRVRGGDGKM | 0.7 | 0.8 | 0.3 | 0.4 | 1189 |
| RRATRRVRGGDGKMK | 0.8 | 0.8 | 0.3 | 0.4 | 1190 |
| RATRRVRGGDGKMKE | 0.7 | 0.8 | 0.3 | 0.4 | 1191 |
| ATRRVRGGDGKMKEL | 0.7 | 0.7 | 0.5 | 0.5 | 1192 |
| TRRVRGGDGKMKELS | 0.8 | 0.7 | 0.5 | 0.5 | 1193 |
| RRVRGGDGKMKELSP | 0.9 | 0.9 | 0.6 | 0.6 | 1194 |
| RVRGGDGKMKELSPR | 0.9 | 0.8 | 0.7 | 0.7 | 1195 |
| VRGGDGKMKELSPRW | 0.8 | 0.6 | 0.7 | 0.7 | 1196 |
| RGGDGKMKELSPRWY | 0.6 | 0.6 | 0.6 | 0.6 | 1197 |
| GGDGKMKELSPRWYF | 0.7 | 0.8 | 0.7 | 0.8 | 1198 |
| GDGKMKELSPRWYFY | 0.6 | 0.6 | 0.8 | 0.7 | 1199 |
| DGKMKELSPRWYFYY | 0.6 | 0.8 | 0.7 | 0.8 | 1200 |
| GKMKELSPRWYFYYL | 0.6 | 0.6 | 0.7 | 0.8 | 1201 |
| KMKELSPRWYFYYLG | 0.5 | 0.6 | 0.6 | 0.8 | 1202 |
| MKELSPRWYFYYLGT | 0.6 | 0.7 | 0.6 | 0.7 | 1203 |
| KELSPRWYFYYLGTG | 0.4 | 0.4 | 0.6 | 0.7 | 1204 |
| ELSPRWYFYYLGTGP | 0.5 | 0.6 | 0.5 | 0.7 | 1205 |
| LSPRWYFYYLGTGPE | 0.8 | 0.7 | 0.6 | 0.5 | 1206 |
| SPRWYFYYLGTGPEA | 0.6 | 0.7 | 0.8 | 0.8 | 1207 |
| PRWYEYYLGTGPEAS | 0.6 | 0.7 | 0.7 | 0.7 | 1208 |
| RWYFYYLGTGPEASL | 0.7 | 0.6 | 0.8 | 0.7 | 1209 |
| WYFYYLGTGPEASLP | 0.6 | 0.7 | 0.6 | 0.6 | 1210 |
| YFYYLGTGPEASLPY | 0.6 | 0.7 | 0.8 | 0.7 | 1211 |
| FYYLGTGPEASLPYG | 0.7 | 0.6 | 0.7 | 0.7 | 1212 |
| YYLGTGPEASLPYGA | 0.7 | 0.7 | 0.8 | 0.8 | 1213 |
| YLGTGPEASLPYGAN | 0.8 | 0.6 | 0.7 | 0.7 | 1214 |
| LGTGPEASLPYGANK | 0.9 | 0.9 | 0.6 | 0.5 | 1215 |
| GTGPEASLPYGANKE | 0.8 | 0.7 | 0.4 | 0.4 | 1216 |
| TGPEASLPYGANKEG | 1.1 | 0.9 | 0.6 | 0.8 | 1217 |
| GPEASLPYGANKEGI | 0.9 | 0.9 | 0.8 | 0.6 | 1218 |
| PEASLPYGANKEGIV | 0.7 | 0.9 | 0.6 | 0.5 | 1219 |
| EASLPYGANKEGIVW | 0.6 | 0.8 | 0.6 | 0.7 | 1220 |
| ASLPYGANKEGIVWV | 0.6 | 0.7 | 0.8 | 0.9 | 1221 |
| SLPYGANKEGIVWVA | 0.7 | 0.9 | 0.6 | 0.6 | 1222 |
| LPYGANKEGIVWVAT | 0.6 | 0.7 | 0.6 | 0.5 | 1223 |
| PYGANKEGIVWVATE | 0.7 | 0.7 | 0.7 | 0.6 | 1224 |
| YGANKEGIVWVATEG | 0.6 | 0.6 | 0.7 | 0.7 | 1225 |
| GANKEGIVWVATEGA | 0.5 | 0.5 | 0.5 | 0.4 | 1226 |
| ANKEGIVWVATEGAL | 0.6 | 0.6 | 0.6 | 0.5 | 1227 |
| NKEGIVWVATEGALN | 0.7 | 0.7 | 0.6 | 0.6 | 1228 |
| KEGIVWVATEGALNT | 0.6 | 0.7 | 0.7 | 0.6 | 1229 |
| EGIVWVATEGALNTP | 0.8 | 0.8 | 0.6 | 0.6 | 1230 |
| GIVWVATEGALNTPK | 1.0 | 1.0 | 0.5 | 0.5 | 1231 |
| IVWVATEGALNTPKD | 0.8 | 0.8 | 0.5 | 0.4 | 1232 |
| VWVATEGALNTPKDH | 0.8 | 0.6 | 0.6 | 0.8 | 1233 |
| WVATEGALNTPKDHI | 0.9 | 0.8 | 0.5 | 0.5 | 1234 |
| VATEGALNTPKDHIG | 0.9 | 0.9 | 0.5 | 0.5 | 1235 |
| ATEGALNTPKDHIGT | 0.7 | 0.8 | 0.5 | 0.5 | 1236 |
| TEGALNTPKDHIGTR | 0.9 | 1.0 | 0.7 | 0.7 | 1237 |
| EGALNTPKDHIGTRN | 0.6 | 0.7 | 0.5 | 0.7 | 1238 |
| GALNTPKDHIGTRNP | 0.6 | 0.7 | 0.4 | 0.5 | 1239 |
| ALNTPKDHIGTRNPN | 0.6 | 0.8 | 0.4 | 0.5 | 1240 |
| LNTPKDHIGTRNPNN | 0.6 | 0.8 | 0.6 | 0.5 | 1241 |
| NTPKDHIGTRNPNNN | 0.7 | 0.8 | 0.6 | 0.5 | 1242 |
| TPKDHIGTRNPNNNA | 0.9 | 0.9 | 0.6 | 0.5 | 1243 |
| PKDHIGTRNPNNNAA | 0.8 | 0.8 | 0.6 | 0.5 | 1244 |
| KDHIGTRNPNNNAAT | 0.8 | 0.9 | 0.6 | 0.6 | 1245 |
| DHIGTRNPNNNAATV | 0.7 | 0.8 | 1.0 | 0.9 | 1246 |
| HIGTRNPNNNAATVL | 0.9 | 0.9 | 1.2 | 1.2 | 1247 |
| IGTRNPNNNAATVLQ | 0.9 | 0.8 | 0.8 | 0.9 | 1248 |
| GTRNPNNNAATVLQL | 0.8 | 0.8 | 0.8 | 1.0 | 1249 |
| TRNPNNNAATVLQLP | 0.8 | 0.7 | 0.7 | 0.8 | 1250 |
| RNPNNNAATVLQLPQ | 0.7 | 0.7 | 0.9 | 0.8 | 1251 |
| NPNNNAATVLQLPQG | 0.9 | 0.9 | 0.9 | 0.8 | 1252 |
| PNNNAATVLQLPQGT | 0.6 | 0.6 | 0.8 | 0.9 | 1253 |
| NNNAATVLQLPQGTT | 0.7 | 0.8 | 0.8 | 0.7 | 1254 |
| NNAATVLQLPQGTTL | 0.9 | 0.9 | 0.8 | 0.8 | 358 |
| NAATVLQLPQGTTLP | 0.5 | 0.7 | 0.4 | 0.6 | 359 |
| 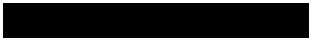 | 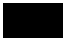 | 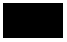 | 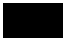 | 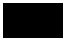 | 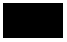 |
| 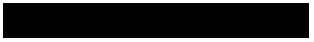 | 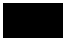 | 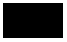 | 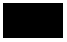 | 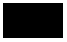 | 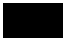 |
| 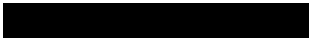 | 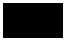 | 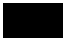 | 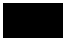 | 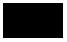 | 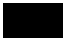 |
| VLQLPQGTTLPKGFY | 0.6 | 0.7 | 0.8 | 0.7 | 363 |
| LQLPQGTTLPKGFYA | 0.8 | 1.0 | 0.5 | 0.5 | 364 |
| QLPQGTTLPKGFYAE | 0.7 | 0.9 | 0.6 | 0.6 | 365 |
| LPQGTTLPKGFYAEG | 0.8 | 0.8 | 0.8 | 0.9 | 366 |
| PQGTTLPKGFYAEGS | 0.6 | 0.7 | 0.6 | 0.6 | 367 |
| QGTTLPKGFYAEGSR | 0.7 | 0.8 | 0.7 | 0.7 | 368 |
| GTTLPKGFYAEGSRG | 0.6 | 0.6 | 0.6 | 0.5 | 369 |
| TTLPKGFYAEGSRGG | 0.7 | 0.6 | 0.5 | 0.6 | 370 |
| TLPKGFYAEGSRGGS | 1.2 | 0.7 | 0.5 | 0.5 | 371 |
| LPKGFYAEGSRGGSQ | 0.6 | 0.6 | 0.5 | 0.6 | 1255 |
| PKGFYAEGSRGGSQA | 0.7 | 0.8 | 0.5 | 0.5 | 1256 |
| KGFYAEGSRGGSQAS | 0.5 | 0.7 | 0.4 | 0.5 | 1257 |
| GFYAEGSRGGSQASS | 0.6 | 0.7 | 0.5 | 0.5 | 1258 |
| FYAEGSRGGSQASSR | 0.7 | 0.8 | 0.8 | 0.6 | 1259 |
| YAEGSRGGSQASSRS | 0.7 | 0.9 | 0.6 | 0.4 | 1260 |
| AEGSRGGSQASSRSS | 0.8 | 0.8 | 0.8 | 0.6 | 1261 |
| EGSRGGSQASSRSSS | 1.0 | 1.0 | 0.8 | 0.7 | 1262 |
| GSRGGSQASSRSSSR | 0.7 | 0.7 | 0.7 | 1.1 | 1263 |
| SRGGSQASSRSSSRS | 0.6 | 0.7 | 0.6 | 0.5 | 1264 |
| RGGSQASSRSSSRSR | 0.8 | 0.8 | 0.6 | 0.4 | 1265 |
| GGSQASSRSSSRSRG | 0.8 | 0.7 | 0.6 | 0.6 | 1266 |
| GSQASSRSSSRSRGN | 0.8 | 0.8 | 0.6 | 0.6 | 1267 |
| SQASSRSSSRSRGNS | 0.7 | 0.7 | 0.6 | 0.6 | 1268 |
| QASSRSSSRSRGNSR | 0.7 | 0.7 | 0.5 | 0.5 | 1269 |
| ASSRSSSRSRGNSRN | 0.7 | 0.6 | 0.6 | 0.6 | 1270 |
| SSRSSSRSRGNSRNS | 0.7 | 0.7 | 0.7 | 0.7 | 1271 |
| SRSSSRSRGNSRNST | 0.7 | 0.7 | 0.6 | 0.6 | 1272 |
| RSSSRSRGNSRNSTP | 0.8 | 0.9 | 0.4 | 0.4 | 1273 |
| SSSRSRGNSRNSTPG | 0.6 | 0.7 | 0.5 | 0.5 | 1274 |
| SSRSRGNSRNSTPGS | 0.5 | 0.6 | 0.5 | 0.5 | 1275 |
| SRSRGNSRNSTPGSS | 0.5 | 0.7 | 0.4 | 0.5 | 1276 |
| RSRGNSRNSTPGSSR | 0.6 | 0.8 | 0.3 | 0.3 | 1277 |
| SRGNSRNSTPGSSRG | 0.8 | 0.9 | 0.4 | 0.4 | 1278 |
| RGNSRNSTPGSSRGN | 0.6 | 0.8 | 0.6 | 0.6 | 1279 |
| GNSRNSTPGSSRGNS | 0.7 | 0.8 | 0.7 | 0.6 | 1280 |
| NSRNSTPGSSRGNSP | 0.7 | 0.9 | 0.7 | 0.7 | 1281 |
| SRNSTPGSSRGNSPA | 0.9 | 0.9 | 0.7 | 0.7 | 1282 |
| RNSTPGSSRGNSPAR | 0.8 | 0.9 | 0.7 | 0.6 | 553 |
| 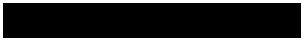 | 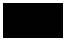 | 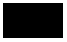 | 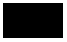 | 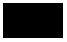 | 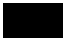 |
| 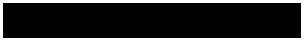 | 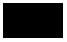 | 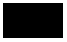 | 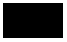 | 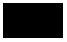 | 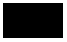 |
| 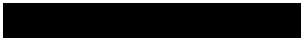 | 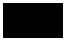 | 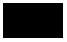 | 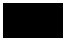 | 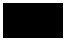 | 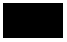 |
| 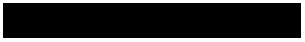 | 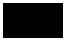 | 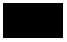 | 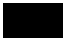 | 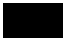 | 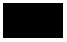 |
| 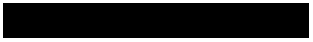 | 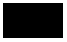 | 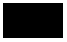 | 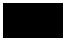 | 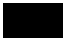 | 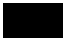 |
| SSRGNSPARMASGGG | 0.9 | 0.8 | 0.9 | 0.8 | 1283 |
| SRGNSPARMASGGGE | 0.8 | 0.8 | 0.4 | 0.5 | 1284 |
| RGNSPARMASGGGET | 0.7 | 0.8 | 0.5 | 0.6 | 1285 |
| GNSPARMASGGGETA | 0.7 | 0.8 | 0.4 | 0.5 | 1286 |
| NSPARMASGGGETAL | 0.7 | 0.9 | 0.4 | 0.3 | 372 |
| SPARMASGGGETALA | 0.8 | 0.9 | 0.2 | 0.1 | 373 |
| PARMASGGGETALAL | 0.8 | 1.0 | 0.9 | 0.7 | 374 |
| ARMASGGGETALALL | 0.7 | 0.8 | 0.8 | 0.6 | 375 |
| RMASGGGETALALLL | 0.5 | 0.6 | 0.8 | 0.8 | 376 |
| MASGGGETALALLLL | 0.6 | 0.7 | 0.9 | 0.7 | 377 |
| ASGGGETALALLLLD | 0.9 | 0.8 | 1.3 | 1.3 | 378 |
| SGGGETALALLLLDR | 0.6 | 0.7 | 0.7 | 0.7 | 1287 |
| GGGETALALLLLDRL | 0.6 | 0.5 | 0.8 | 0.7 | 1288 |
| GGETALALLLLDRLN | 0.6 | 0.7 | 0.8 | 0.8 | 1289 |
| GETALALLLLDRLNQ | 0.6 | 0.6 | 0.7 | 0.7 | 1290 |
| ETALALLLLDRLNQL | 0.5 | 0.5 | 0.7 | 0.8 | 1291 |
| TALALLLLDRLNQLE | 0.7 | 0.7 | 0.8 | 0.8 | 1292 |
| ALALLLLDRLNQLES | 0.7 | 0.7 | 0.8 | 0.8 | 1293 |
| LALLLLDRLNQLESK | 0.6 | 0.7 | 0.6 | 0.7 | 1294 |
| ALLLLDRLNQLESKV | 0.7 | 0.8 | 0.8 | 0.8 | 1295 |
| LLLLDRLNQLESKVS | 0.6 | 0.7 | 0.4 | 0.4 | 1296 |
| LLLDRLNQLESKVSG | 0.9 | 0.8 | 0.5 | 0.5 | 1297 |
| LLDRLNQLESKVSGK | 0.6 | 0.7 | 0.2 | 0.3 | 1298 |
| LDRLNQLESKVSGKG | 0.8 | 1.0 | 0.5 | 0.5 | 1299 |
| DRLNQLESKVSGKGQ | 0.7 | 0.9 | 0.6 | 0.6 | 1300 |
| RLNQLESKVSGKGQQ | 0.7 | 0.8 | 0.7 | 0.6 | 1301 |
| LNQLESKVSGKGQQQ | 0.7 | 0.8 | 0.7 | 0.7 | 1302 |
| NQLESKVSGKGQQQQ | 0.9 | 0.8 | 0.7 | 0.7 | 1303 |
| QLESKVSGKGQQQQG | 0.8 | 0.8 | 0.9 | 1.0 | 1304 |
| LESKVSGKGQQQQGQ | 0.7 | 0.8 | 0.7 | 0.8 | 1305 |
| ESKVSGKGQQQQGQT | 0.8 | 0.7 | 0.9 | 1.1 | 1306 |
| SKVSGKGQQQQGQTV | 0.7 | 0.6 | 0.8 | 0.8 | 1307 |
| KVSGKGQQQQGQTVT | 0.7 | 0.8 | 0.5 | 0.6 | 1308 |
| VSGKGQQQQGQTVTK | 1.4 | 1.0 | 0.9 | 0.7 | 1309 |
| SGKGQQQQGQTVTKK | 1.1 | 1.0 | 0.6 | 0.6 | 1310 |
| GKGQQQQGQTVTKKS | 1.0 | 0.9 | 0.7 | 0.6 | 1311 |
| KGQQQQGQTVTKKSA | 0.9 | 1.0 | 0.4 | 0.4 | 1312 |
| GQQQQGQTVTKKSAA | 0.9 | 0.9 | 0.5 | 0.5 | 1313 |
| QQQQGQTVTKKSAAE | 0.6 | 0.6 | 0.2 | 0.2 | 1314 |
| QQQGQTVTKKSAAEA | 0.5 | 0.6 | 0.6 | 0.6 | 1315 |
| QQGQTVTKKSAAEAS | 0.7 | 0.8 | 0.5 | 0.5 | 379 |
| QGQTVTKKSAAEASK | 1.0 | 1.1 | 0.4 | 0.4 | 380 |
| GQTVTKKSAAEASKK | 0.7 | 0.7 | 0.3 | 0.4 | 381 |
| QTVTKKSAAEASKKP | 0.9 | 0.8 | 0.5 | 0.5 | 382 |
| TVTKKSAAEASKKPR | 1.0 | 1.0 | 0.3 | 0.4 | 383 |
| VTKKSAAEASKKPRQ | 0.9 | 0.9 | 0.7 | 0.6 | 384 |
| TKKSAAEASKKPRQK | 0.9 | 0.8 | 0.4 | 0.4 | 385 |
| KKSAAEASKKPRQKR | 1.0 | 1.0 | 0.5 | 0.5 | 386 |
| KSAAEASKKPRQKRT | 0.7 | 0.7 | 0.4 | 0.4 | 387 |
| SAAEASKKPRQKRTA | 0.8 | 0.8 | 0.4 | 0.4 | 388 |
| AAEASKKPRQKRTAT | 0.9 | 0.8 | 0.5 | 0.5 | 389 |
| AEASKKPRQKRTATK | 0.8 | 0.8 | 0.4 | 0.4 | 1316 |
| EASKKPRQKRTATKQ | 0.8 | 0.9 | 0.6 | 0.8 | 1317 |
| ASKKPRQKRTATKQY | 0.6 | 0.7 | 0.5 | 0.6 | 1318 |
| SKKPRQKRTATKQYN | 0.7 | 0.8 | 0.5 | 0.6 | 1319 |
| KKPRQKRTATKQYNV | 0.7 | 0.6 | 0.5 | 0.4 | 1320 |
| KPRQKRTATKQYNVT | 0.7 | 0.8 | 0.4 | 0.4 | 390 |
| PRQKRTATKQYNVTQ | 0.9 | 1.0 | 0.9 | 1.0 | 391 |
| RQKRTATKQYNVTQA | 0.8 | 0.9 | 0.8 | 0.9 | 392 |
| QKRTATKQYNVTQAF | 0.7 | 0.8 | 0.8 | 0.8 | 393 |
| KRTATKQYNVTQAFG | 0.8 | 0.7 | 0.6 | 0.6 | 394 |
| RTATKQYNVTQAFGR | 0.8 | 0.9 | 0.9 | 0.8 | 395 |
| TATKQYNVTQAFGRR | 0.8 | 0.8 | 1.0 | 0.9 | 396 |
| ATKQYNVTQAFGRRG | 0.8 | 0.9 | 0.8 | 0.8 | 565 |
| TKQYNVTQAFGRRGP | 0.8 | 0.8 | 0.9 | 0.9 | 566 |
| KQYNVTQAFGRRGPE | 0.7 | 0.6 | 0.5 | 0.5 | 567 |
| QYNVTQAFGRRGPEQ | 0.6 | 0.7 | 0.8 | 0.8 | 568 |
| YNVTQAFGRRGPEQT | 0.6 | 0.7 | 0.5 | 0.5 | 569 |
| NVTQAFGRRGPEQTQ | 0.7 | 0.7 | 0.5 | 0.5 | 570 |
| VTQAFGRRGPEQTQG | 0.7 | 0.8 | 0.5 | 0.6 | 571 |
| TQAFGRRGPEQTQGN | 0.7 | 0.8 | 0.6 | 0.7 | 572 |
| QAFGRRGPEQTQGNF | 0.7 | 0.9 | 0.4 | 0.4 | 1321 |
| AFGRRGPEQTQGNFG | 0.5 | 0.6 | 0.3 | 0.4 | 1322 |
| FGRRGPEQTQGNFGD | 0.6 | 0.7 | 0.3 | 0.4 | 397 |
| GRRGPEQTQGNFGDQ | 0.6 | 0.6 | 0.6 | 0.6 | 398 |
| RRGPEQTQGNFGDQD | 0.6 | 0.6 | 0.4 | 0.3 | 399 |
| RGPEQTQGNFGDQDL | 0.7 | 0.7 | 0.6 | 0.4 | 400 |
| GPEQTQGNFGDQDLI | 0.8 | 0.8 | 0.6 | 0.5 | 401 |
| PEQTQGNFGDQDLIR | 0.9 | 0.8 | 0.6 | 0.5 | 402 |
| EQTQGNFGDQDLIRQ | 1.0 | 1.0 | 0.6 | 0.6 | 403 |
| QTQGNFGDQDLIRQG | 0.9 | 0.9 | 0.7 | 0.8 | 404 |
| TQGNFGDQDLIRQGT | 0.9 | 0.8 | 0.8 | 0.8 | 1323 |
| QGNFGDQDLIRQGTD | 0.7 | 0.6 | 0.6 | 0.5 | 1324 |
| GNFGDQDLIRQGTDY | 0.6 | 0.7 | 0.9 | 0.9 | 1325 |
| NFGDQDLIRQGTDYK | 0.7 | 0.8 | 0.4 | 0.5 | 1326 |
| FGDQDLIRQGTDYKH | 0.7 | 0.8 | 0.7 | 0.6 | 1327 |
| GDQDLIRQGTDYKHW | 0.8 | 0.9 | 0.5 | 0.6 | 1328 |
| DQDLIRQGTDYKHWP | 0.7 | 0.8 | 0.4 | 0.4 | 1329 |
| QDLIRQGTDYKHWPQ | 0.6 | 0.7 | 0.5 | 0.6 | 1330 |
| DLIRQGTDYKHWPQI | 0.5 | 0.6 | 0.5 | 0.5 | 1331 |
| LIRQGTDYKHWPQIA | 0.7 | 0.7 | 0.5 | 0.6 | 1332 |
| IRQGTDYKHWPQIAQ | 0.7 | 0.7 | 0.6 | 0.5 | 1333 |
| RQGTDYKHWPQIAQF | 0.7 | 0.7 | 0.8 | 0.7 | 1334 |
| QGTDYKHWPQIAQFA | 0.6 | 0.8 | 0.7 | 0.6 | 1335 |
| GTDYKHWPQIAQFAP | 0.6 | 0.8 | 0.7 | 0.8 | 1336 |
| TDYKHWPQIAQFAPS | 0.8 | 0.8 | 1.1 | 1.2 | 1337 |
| DYKHWPQIAQFAPSA | 0.8 | 0.9 | 0.7 | 0.6 | 1338 |
| YKHWPQIAQFAPSAS | 0.7 | 0.9 | 0.8 | 0.8 | 1339 |
| KHWPQIAQFAPSASA | 0.7 | 0.8 | 0.6 | 0.7 | 1340 |
| HWPQIAQFAPSASAF | 0.7 | 0.8 | 0.8 | 0.8 | 1341 |
| WPQIAQFAPSASAFF | 0.6 | 0.6 | 0.9 | 0.8 | 1342 |
| PQIAQFAPSASAFFG | 0.5 | 0.7 | 0.7 | 0.8 | 1343 |
| QIAQFAPSASAFFGM | 0.7 | 0.9 | 0.7 | 0.9 | 1344 |
| IAQFAPSASAFFGMS | 0.6 | 0.7 | 0.6 | 0.8 | 1345 |
| AQFAPSASAFFGMSR | 0.5 | 0.7 | 0.6 | 0.8 | 1346 |
| QFAPSASAFFGMSRI | 0.5 | 0.7 | 0.6 | 0.6 | 1347 |
| FAPSASAFFGMSRIG | 0.6 | 0.5 | 0.4 | 0.4 | 1348 |
| APSASAFFGMSRIGM | 0.5 | 0.7 | 0.6 | 0.8 | 1349 |
| PSASAFFGMSRIGME | 0.6 | 0.6 | 0.5 | 0.4 | 1350 |
| SASAFFGMSRIGMEV | 0.6 | 0.7 | 0.7 | 0.7 | 1351 |
| ASAFFGMSRIGMEVT | 0.7 | 0.7 | 0.4 | 0.5 | 1352 |
| SAFFGMSRIGMEVTP | 0.7 | 0.8 | 0.5 | 0.6 | 1353 |
| AFFGMSRIGMEVTPS | 0.6 | 0.7 | 0.8 | 0.6 | 1354 |
| FFGMSRIGMEVTPSG | 0.7 | 0.8 | 0.5 | 0.5 | 1355 |
| FGMSRIGMEVTPSGT | 0.7 | 0.8 | 0.5 | 0.5 | 1356 |
| GMSRTGMEVTPSGTW | 0.6 | 0.6 | 0.5 | 0.6 | 1357 |
| MSRIGMEVTPSGTWL | 0.7 | 0.8 | 0.7 | 0.7 | 1358 |
| SRIGMEVTPSGTWLT | 0.6 | 0.8 | 0.6 | 0.6 | 1359 |
| RIGMEVTPSGTWLTY | 0.5 | 0.5 | 0.7 | 0.8 | 1360 |
| IGMEVTPSGTWLTYH | 0.6 | 0.8 | 0.8 | 0.8 | 1361 |
| GMEVTPSGTWLTYHG | 0.6 | 0.6 | 0.7 | 0.7 | 1362 |
| MEVTPSGTWLTYHGA | 0.6 | 0.8 | 0.8 | 0.7 | 1363 |
| EVTPSGTWLTYHGAI | 0.6 | 0.7 | 0.7 | 0.8 | 1364 |
| VTPSGTWLTYHGAIK | 0.6 | 0.8 | 0.3 | 0.3 | 1365 |
| TPSGTWLTYHGAIKL | 0.6 | 0.8 | 0.5 | 0.7 | 1366 |
| PSGTWLTYHGAIKLD | 0.6 | 0.7 | 0.4 | 0.4 | 1367 |
| SGTWLTYHGAIKLDD | 0.5 | 0.5 | 0.8 | 1.0 | 1368 |
| GTWLTYHGAIKLDDK | 0.6 | 0.6 | 0.4 | 0.4 | 1369 |
| TWLTYHGAIKLDDKD | 0.6 | 0.6 | 0.4 | 0.4 | 1370 |
| WLTYHGAIKLDDKDP | 0.7 | 0.8 | 0.4 | 0.4 | 1371 |
| LTYHGAIKLDDKDPQ | 0.6 | 0.7 | 0.5 | 0.5 | 1372 |
| TYHGAIKLDDKDPQF | 0.7 | 0.8 | 0.4 | 0.4 | 1373 |
| YHGAIKLDDKDPQFK | 0.9 | 0.9 | 0.3 | 0.4 | 1374 |
| HGAIKLDDKDPQFKD | 0.6 | 0.6 | 0.4 | 0.4 | 1375 |
| GAIKLDDKDPQFKDN | 0.7 | 0.7 | 0.5 | 0.6 | 1376 |
| AIKLDDKDPQFKDNV | 0.8 | 0.7 | 0.5 | 0.5 | 1377 |
| IKLDDKDPQFKDNVI | 0.9 | 0.9 | 0.6 | 0.6 | 405 |
| KLDDKDPQFKDNVIL | 0.7 | 0.8 | 0.8 | 0.7 | 406 |
| 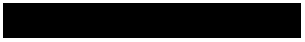 | 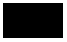 | 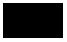 | 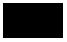 | 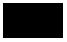 | 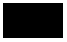 |
| 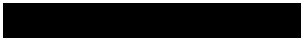 | 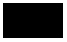 | 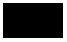 | 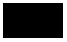 | 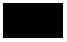 | 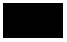 |
| 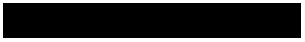 | 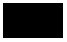 | 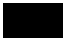 | 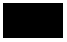 | 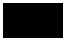 | 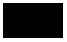 |
| 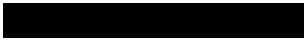 | 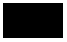 | 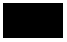 | 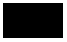 | 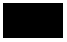 | 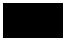 |
| 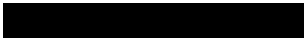 | 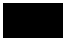 | 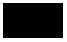 | 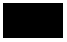 | 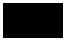 | 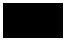 |
| PQFKDNVILLNKHID | 0.7 | 0.7 | 0.8 | 0.7 | 412 |
| QFKDNVILLNKHIDA | 0.6 | 0.6 | 0.8 | 0.9 | 413 |
| FKDNVILLNKHIDAY | 0.5 | 0.6 | 0.8 | 0.8 | 1378 |
| KDNVILLNKHIDAYK | 0.7 | 0.8 | 0.5 | 0.5 | 1379 |
| DNVILLNKHIDAYKT | 0.6 | 0.5 | 0.7 | 0.7 | 1380 |
| NVILLNKHIDAYKTF | 0.7 | 0.7 | 0.7 | 0.8 | 1381 |
| VILLNKHIDAYKTFP | 0.8 | 0.7 | 0.4 | 0.5 | 1382 |
| ILLNKHIDAYKTFPP | 0.7 | 0.7 | 0.7 | 0.6 | 1383 |
| LLNKHIDAYKTFPPT | 0.7 | 0.6 | 0.5 | 0.5 | 1384 |
| LNKHIDAYKTFPPTE | 0.5 | 0.5 | 0.4 | 0.4 | 1385 |
| NKHIDAYKTFPPTEP | 0.5 | 0.6 | 0.4 | 0.5 | 1386 |
| KHIDAYKTFPPTEPK | 0.7 | 0.8 | 0.3 | 0.3 | 1387 |
| HIDAYKTFPPTEPKK | 0.7 | 0.6 | 0.3 | 0.4 | 1388 |
| IDAYKTFPPTEPKKD | 0.6 | 0.7 | 0.3 | 0.4 | 1389 |
| DAYKTFPPTEPKKDK | 0.6 | 0.8 | 0.2 | 0.2 | 1390 |
| AYKTFPPTEPKKDKK | 0.6 | 0.7 | 0.1 | 0.3 | 1391 |
| YKTFPPTEPKKDKKK | 0.6 | 0.7 | 0.3 | 0.3 | 1392 |
| KTFPPTEPKKDKKKK | 0.7 | 0.6 | 0.3 | 0.3 | 1393 |
| TFPPTEPKKDKKKKT | 0.7 | 0.6 | 0.4 | 0.5 | 1394 |
| FPPTEPKKDKKKKTD | 0.6 | 0.6 | 0.3 | 0.4 | 1395 |
| PPTEPKKDKKKKTDE | 0.7 | 0.6 | 0.3 | 0.4 | 1396 |
| PTEPKKDKKKKTDEA | 0.6 | 0.6 | 0.3 | 0.4 | 1397 |
| TEPKKDKKKKTDEAQ | 0.8 | 0.7 | 0.4 | 0.5 | 1398 |
| EPKKDKKKKTDEAQP | 0.8 | 0.8 | 0.3 | 0.5 | 1399 |
| PKKDKKKKTDEAQPL | 0.8 | 0.8 | 0.3 | 0.4 | 1400 |
| KKDKKKKTDEAQPLP | 0.6 | 0.7 | 0.4 | 0.5 | 1401 |
| KDKKKKTDEAQPLPQ | 0.6 | 0.6 | 0.4 | 0.5 | 1402 |
| DKKKKTDEAQPLPQR | 0.6 | 0.8 | 0.4 | 0.5 | 1403 |
| KKKKTDEAQPLPQRQ | 0.9 | 1.0 | 0.4 | 0.6 | 1404 |
| KKKTDEAQPLPQRQK | 0.8 | 0.9 | 0.3 | 0.4 | 1405 |
| KKTDEAQPLPQRQKK | 0.7 | 0.7 | 0.3 | 0.4 | 1406 |
| KTDEAQPLPQRQKKQ | 0.9 | 0.7 | 0.4 | 0.4 | 1407 |
| TDEAQPLPQRQKKQP | 0.6 | 0.7 | 0.2 | 0.5 | 1408 |
| DEAQPLPQRQKKQPT | 0.8 | 0.7 | 0.3 | 0.3 | 1409 |
| EAQPLPQRQKKQPTV | 0.7 | 0.6 | 0.4 | 0.4 | 1410 |
| AQPLPQRQKKQPTVT | 0.7 | 0.6 | 0.3 | 0.4 | 1411 |
| QPLPQRQKKQPTVTL | 0.5 | 0.6 | 0.9 | 1.0 | 414 |
| PLPQRQKKQPTVTLL | 0.5 | 0.7 | 1.1 | 1.1 | 415 |
| LPQRQKKQPTVTLLP | 0.7 | 0.8 | 0.7 | 0.8 | 416 |
| PQRQKKQPTVTLLPA | 0.7 | 0.8 | 0.7 | 0.9 | 417 |
| QRQKKQPTVTLLPAA | 0.7 | 0.8 | 0.8 | 1.1 | 418 |
| RQKKQPTVTLLPAAD | 0.8 | 0.7 | 0.5 | 0.6 | 419 |
| QKKQPTVTLLPAADM | 0.6 | 0.7 | 0.7 | 0.9 | 420 |
| KKQPTVTLLPAADMD | 0.6 | 0.7 | 0.3 | 0.3 | 1412 |
| KQPTVTLLPAADMDD | 0.7 | 0.7 | 0.3 | 0.4 | 1413 |
| QPTVTLLPAADMDDF | 0.5 | 0.7 | 0.5 | 0.5 | 1414 |
| PTVTLLPAADMDDFS | 0.7 | 0.8 | 0.3 | 0.3 | 1415 |
| TVTLLPAADMDDFSR | 0.6 | 0.6 | 0.3 | 0.3 | 1416 |
| VTLLPAADMDDFSRQ | 0.5 | 0.5 | 0.4 | 0.1 | 1417 |
| TLLPAADMDDFSRQL | 0.8 | 0.7 | 0.4 | 0.5 | 1418 |
| LLPAADMDDFSRQLQ | 0.6 | 0.7 | 0.4 | 0.4 | 1419 |
| LPAADMDDFSRQLQN | 0.7 | 0.7 | 0.4 | 0.4 | 1420 |
| PAADMDDFSRQLQNS | 0.8 | 0.8 | 0.3 | 0.3 | 1421 |
| AADMDDFSRQLQNSM | 0.7 | 0.8 | 0.3 | 0.4 | 1422 |
| ADMDDFSRQLQNSMS | 0.7 | 0.8 | 0.2 | 0.4 | 1423 |
| DMDDFSRQLQNSMSG | 0.6 | 0.8 | 0.3 | 0.4 | 1424 |
| MDDFSRQLQNSMSGA | 0.7 | 1.0 | 0.5 | 0.5 | 1425 |
| DDFSRQLQNSMSGAS | 0.6 | 0.8 | 0.3 | 0.3 | 1426 |
| DFSRQLQNSMSGASA | 0.6 | 0.6 | 0.4 | 0.5 | 1427 |
| FSRQLQNSMSGASAD | 0.6 | 0.7 | 0.2 | 0.4 | 1428 |
| SRQLQNSMSGASADS | 0.7 | 0.8 | 0.3 | 0.5 | 1429 |
| RQLQNSMSGASADST | 0.6 | 0.7 | 0.2 | 0.4 | 1430 |
| QLQNSMSGASADSTQ | 0.6 | 0.8 | 0.4 | 0.5 | 1431 |
| LQNSMSGASADSTQA | 0.5 | 0.7 | 0.2 | 0.0 | 1432 |

TABLE 25
Binding of a rabbit serum to linear and looped/cyclic
peptides of protein X1 of SARS-CoV Urbani.

| | Rabbit serum | Rabbit serum | |
|---|---|---|---|
| Peptide | linear | looped | SEQ |
| sequence | peptides | peptides | ID NO |
| MDLFMRFFTLGSITA | 0.5 | 0.5 | 607 |
| DLFMRFFTLGSITAQ | 0.1 | 0.3 | 608 |
| LFMRFFTLGSITAQP | 0.5 | 0.4 | 609 |
| FMRFFTLGSITAQPV | 0.3 | 0.4 | 610 |
| MRFFTLGSITAQPVK | 0.4 | 0.2 | 611 |
| RFFTLGSITAQPVKI | 1.7 | 0.5 | 9 |
| FFTLGSITAQPVKID | 1.1 | 0.0 | 10 |
| FTLGSITAQPVKIDN | 0.7 | 0.8 | 11 |
| TLGSITAQPVKIDNA | 0.8 | 0.4 | 12 |
| LGSITAQPVKIDNAS | 0.3 | 0.4 | 13 |
| GSITAQPVKIDNASP | 0.2 | 0.4 | 14 |
| SITAQPVKIDNASPA | 0.1 | 0.4 | 15 |
| ITAQPVKIDNASPAS | 0.1 | 0.3 | 16 |
| TAQPVKIDNASPAST | 0.1 | 0.4 | 17 |
| AQPVKIDNASPASTV | 0.1 | 0.5 | 18 |
| QPVKIDNASPASTVH | 0.1 | 0.4 | 19 |
| PVKIDNASPASTVHA | 0.1 | 0.4 | 20 |
| VKIDNASPASTVHAT | 0.2 | 0.3 | 21 |
| KIDNASPASTVHATA | 0.2 | 0.3 | 22 |
| IDNASPASTVHATAT | 0.5 | 0.3 | 23 |
| DNASPASTVHATATI | 0.7 | 0.3 | 24 |
| NASPASTVHATATIP | 0.6 | 0.3 | 25 |
| ASPASTVHATATIPL | 1.4 | 0.5 | 26 |
| SPASTVHATATIPLQ | 1.0 | 0.4 | 27 |
| PASTVHATATIPLQA | 0.9 | 0.5 | 28 |
| ASTVHATATIPLQAS | 0.9 | 0.6 | 29 |
| STVHATATIPLQASL | 0.6 | 0.5 | 30 |
| TVHATATIPLQASLP | 0.4 | 0.5 | 31 |
| VHATATIPLQASLPF | 0.1 | 0.6 | 32 |
| HATATIPLQASLPFG | 0.1 | 0.5 | 612 |
| ATATIPLQASLPFGW | 0.1 | 0.6 | 613 |
| TATIPLQASLPFGWL | 0.1 | 0.6 | 614 |
| ATIPLQASLPFGWLV | 0.1 | 0.5 | 615 |
| TIPLQASLPFGWLVI | 0.1 | 0.5 | 616 |
| IPLQASLPFGWLVIG | 0.1 | 0.5 | 617 |
| PLQASLPFGWLVIGV | 0.1 | 0.4 | 618 |
| LQASLPFGWLVIGVA | 0.5 | 0.4 | 619 |
| QASLPFGWLVIGVAF | 0.3 | 0.4 | 620 |
| ASLPFGWLVIGVAFL | 1.4 | 0.3 | 621 |
| SLPFGWLVIGVAFLA | 0.4 | 0.1 | 622 |
| LPFGWLVIGVAFLAV | 2.0 | 0.5 | 623 |
| PFGWLVIGVAFLAVF | 1.1 | 0.5 | 624 |
| FGWLVIGVAFLAVFQ | 1.2 | 0.5 | 625 |
| GWLVIGVAFLAVFQS | 0.8 | 0.6 | 626 |
| WLVIGVAFLAVFQSA | 0.5 | 0.5 | 627 |
| LVIGVAFLAVFQSAT | 0.3 | 1.2 | 628 |
| VIGVAFLAVFQSATK | 0.1 | 0.6 | 629 |
| IGVAFLAVFQSATKI | 0.1 | 0.6 | 630 |
| GVAFLAVFQSATKII | 0.9 | 0.6 | 631 |
| VAFLAVFQSATKIIA | 0.2 | 0.6 | 632 |
| AFLAVFQSATKIIAL | 0.2 | 0.8 | 633 |
| FLAVFQSATKIIALN | 0.6 | 0.6 | 634 |
| LAVFQSATKIIALNK | 0.1 | 0.7 | 635 |
| 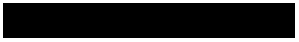 | 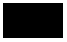 | 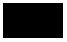 | 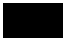 |
| 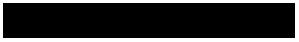 | 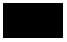 | 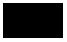 | 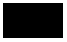 |
| 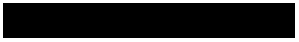 | 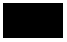 | 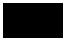 | 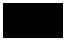 |
| 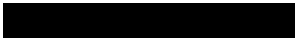 | 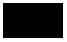 | 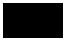 | 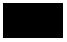 |
| 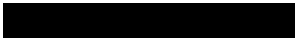 | 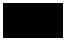 | 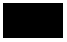 | 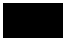 |
| 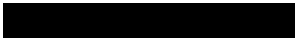 | 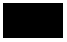 | 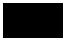 | 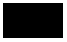 |
| 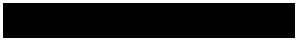 | 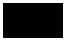 | 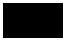 | 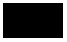 |
| 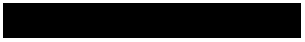 | 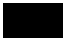 | 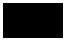 | 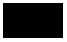 |
| 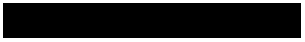 | 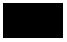 | 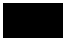 | 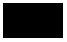 |
| 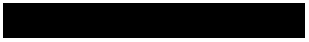 | 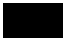 | 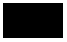 | 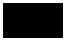 |
| 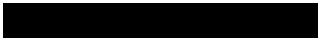 | 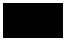 | 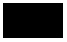 | 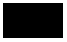 |
| 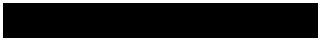 | 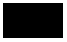 | 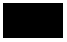 | 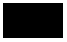 |
| 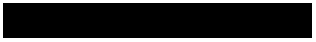 | 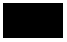 | 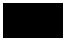 | 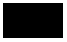 |
| KRWQLALYKGFQFIC | 0.1 | 0.8 | 636 |
| RWQLALYKGFQFICN | 0.3 | 0.9 | 637 |
| WQLALYKGFQFICNL | 0.5 | 0.6 | 638 |
| QLALYKGFQFICNLL | 0.3 | 0.6 | 639 |
| LALYKGFQFICNLLL | 0.2 | 0.6 | 640 |
| ALYKGFQFICNLLLL | 0.6 | 0.4 | 641 |
| LYKGFQFICNLLLLF | 0.2 | 0.3 | 642 |
| YKGFQFICNLLLLFV | 0.3 | 0.0 | 643 |
| KGFQFICNLLLLFVT | 0.6 | 0.5 | 644 |
| GFQFIGNLLLLFVTI | 0.9 | 0.5 | 645 |
| FQFICNLLLLFVTIY | 0.9 | 0.0 | 646 |
| QFICNLLLLFVTIYS | 0.3 | 0.5 | 647 |
| FICNLLLLFVTIYSH | 0.1 | 0.5 | 648 |
| ICNLLLLFVTIYSHL | 0.2 | 0.5 | 649 |
| CNLLLLFVTIYSHLL | 0.1 | 0.6 | 650 |
| NLLLLFVTIYSHLLL | 0.2 | 0.6 | 651 |
| LLLLFVTIYSHLLLV | 0.1 | 0.5 | 652 |
| LLLFVTIYSHLLLVA | 0.1 | 0.5 | 653 |
| LLFVTIYSHLLLVAA | 0.1 | 0.5 | 654 |
| LFVTIYSHLLLVAAG | 0.1 | 0.6 | 655 |
| FVTIYSHLLLVAAGM | 0.1 | 0.5 | 656 |
| VTIYSHLLLVAAGME | 0.1 | 0.5 | 657 |
| TIYSHLLLVAAGMEA | 0.1 | 0.4 | 658 |
| IYSHLLLVAAGMEAQ | 0.1 | 0.4 | 659 |
| YSHLLLVAAGMEAQF | 0.1 | 0.4 | 660 |
| SHLLLVAAGMEAQFL | 0.5 | 0.0 | 661 |
| HLLLVAAGMEAQFLY | 0.5 | 0.4 | 662 |
| LLLVAAGMEAQFLYL | 0.2 | 0.5 | 663 |
| LLVAAGMEAQFLYLY | 0.2 | 0.5 | 664 |
| LVAAGMEAQFLYLYA | 0.1 | 0.6 | 665 |
| VAAGMEAQFLYLYAL | 0.1 | 0.5 | 666 |
| AAGMEAQFLYLYALI | 0.1 | 0.6 | 667 |
| AGMEAQFLYLYALIY | 0.1 | 0.6 | 668 |
| GMEAQFLYLYALIYF | 0.1 | 0.6 | 669 |
| MEAQFLYLYALIYFL | 0.1 | 0.5 | 670 |
| EAQFLYLYALIYFLQ | 0.1 | 0.5 | 671 |
| AQFLYLYALIYFLQC | 0.2 | 0.5 | 672 |
| QFLYLYALIYFLQCI | 0.1 | 0.4 | 673 |
| FLYLYALIYFLQCIN | 0.1 | 0.4 | 674 |
| LYLYALIYFLQCINA | 0.1 | 0.5 | 675 |
| YLYALIYFLQCINAC | 0.1 | 0.4 | 676 |
| LYALIYFLQCINACR | 0.2 | 0.6 | 677 |
| YALIYFLQCINACRI | 0.1 | 0.0 | 678 |
| ALIYFLQCINACRII | 0.7 | 0.6 | 679 |
| LIYFLQCINACRIIM | 0.1 | 0.3 | 680 |
| IYFLQCINACRIIMR | 0.5 | 0.9 | 681 |
| YFLQCINACRIIMRC | 0.1 | 0.8 | 682 |
| FLQCINACRIIMRCW | 0.1 | 0.8 | 683 |
| 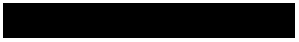 | 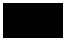 | 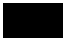 | 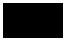 |
| 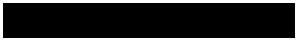 | 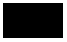 | 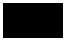 | 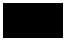 |
| 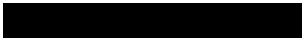 | 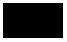 | 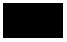 | 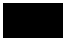 |
| 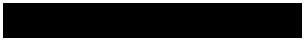 | 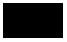 | 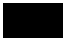 | 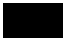 |
| 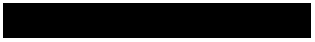 | 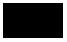 | 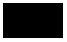 | 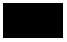 |
| 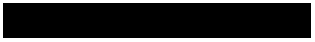 | 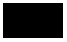 | 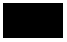 | 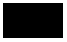 |
| CRIIMRCWLCWKCKS | 0.1 | 0.7 | 36 |
| RIIMRCWLCWKCKSK | 0.1 | 0.3 | 37 |
| IIMRCWLCWKCKSKN | 0.2 | 0.7 | 38 |
| IMRCWLCWKGKSKNP | 0.1 | 0.3 | 39 |
| MRCWLCWKCKSKNPL | 0.1 | 0.5 | 40 |
| RCWLCWKCKSKNPLL | 0.1 | 0.7 | 41 |
| CWLCWKCKSKNPLLY | 0.2 | 0.7 | 42 |
| WLCWKCKSKNPLLYD | 0.2 | 0.6 | 43 |
| LCWKCKSKNPLLYDA | 0.3 | 0.8 | 44 |
| CWKCKSKNPLLYDAN | 0.1 | 0.5 | 45 |
| WKCKSKNPLLYDANY | 0.1 | 0.6 | 684 |
| KCKSKNPLLYDANYF | 0.2 | 0.6 | 685 |
| CKSKNPLLYDANYFV | 0.1 | 0.6 | 686 |
| KSKNPLLYDANYFVC | 0.1 | 0.6 | 687 |
| SKNPLLYDANYFVCW | 0.1 | 0.6 | 688 |
| KNPLLYDANYFVCWH | 0.1 | 0.7 | 689 |
| NPLLYDANYFVCWHT | 0.2 | 0.6 | 690 |
| PLLYDANYFVCWHTH | 0.1 | 0.5 | 691 |
| LLYDANYFVCWHTHN | 0.1 | 0.5 | 692 |
| LYDANYFVCWHTHNY | 0.1 | 0.5 | 693 |
| YDANYFVCWHTHNYD | 0.1 | 0.4 | 46 |
| DANYFVCWHTHNYDY | 0.1 | 0.5 | 47 |
| ANYFVCWHTHNYDYC | 0.1 | 0.5 | 48 |
| NYFVCWHTHNYDYCI | 0.1 | 0.5 | 49 |
| YFVCWHTHNYDYCIP | 0.1 | 0.6 | 50 |
| FVCWHTHNYDYCIPY | 0.1 | 0.7 | 51 |
| VCWHTHNYDYCIPYN | 0.1 | 0.6 | 52 |
| CWHTHNYDYCIPYNS | 0.1 | 0.7 | 53 |
| WHTHNYDYCIPYNSV | 0.1 | 0.6 | 54 |
| HTHNYDYCIPYNSVT | 0.1 | 0.6 | 55 |
| THNYDYCIPYNSVTD | 0.1 | 0.6 | 56 |
| HNYDYCIPYNSVTDT | 0.1 | 0.5 | 57 |
| NYDYCIPYNSVTDTI | 0.1 | 0.5 | 58 |
| YDYCIPYNSVTDTIV | 0.1 | 0.5 | 59 |
| DYCIPYNSVTDTIVV | 0.1 | 0.5 | 60 |
| YCIPYNSVTDTIVVT | 0.1 | 0.5 | 61 |
| CIPYNSVTDTIVVTE | 0.1 | 0.4 | 694 |
| IPYNSVTDTIVVTEG | 0.1 | 0.3 | 695 |
| PYNSVTDTIVVTEGD | 0.1 | 0.3 | 696 |
| YNSVTDTIVVTEGDG | 0.1 | 0.5 | 697 |
| NSVTDTIVVTEGDGI | 0.1 | 0.5 | 698 |
| SVTDTIVVTEGDGIS | 0.1 | 0.4 | 699 |
| VTDTIVVTEGDGIST | 0.1 | 0.5 | 700 |
| TDTIVVTEGDGISTP | 0.1 | 0.3 | 701 |
| DTIVVTEGDGISTPK | 0.1 | 0.5 | 702 |
| TIVVTEGDGISTPKL | 0.1 | 0.5 | 703 |
| IVVTEGDGISTPKLK | 0.1 | 0.4 | 704 |
| VVTEGDGISTPKLKE | 0.1 | 0.4 | 705 |
| VTEGDGISTPKLKED | 0.0 | 0.4 | 706 |
| TEGDGISTPKLKEDY | 0.1 | 0.5 | 707 |
| EGDGISTPKLKEDYQ | 0.1 | 0.4 | 708 |
| GDGISTPKLKEDYQI | 0.1 | 0.5 | 62 |
| DGISTPKLKEDYQIG | 0.1 | 0.4 | 63 |
| GISTPKLKEDYQIGG | 0.1 | 0.3 | 64 |
| ISTPKLKEDYQIGGY | 0.1 | 0.4 | 65 |
| STPKLKEDYQIGGYS | 0.1 | 0.3 | 66 |
| TPKLKEDYQIGGYSE | 0.1 | 1.2 | 67 |
| PKLKEDYQIGGYSED | 0.1 | 0.6 | 68 |
| KLKEDYQIGGYSEDR | 0.1 | 0.5 | 69 |
| LKEDYQIGGYSEDRH | 0.1 | 0.7 | 70 |
| KEDYQIGGYSEDRHS | 0.1 | 0.5 | 71 |
| EDYQIGGYSEDRHSG | 0.1 | 0.6 | 72 |
| DYQIGGYSEDRHSGV | 0.1 | 0.5 | 73 |
| YQIGGYSEDRHSGVK | 0.1 | 0.5 | 74 |
| QIGGYSEDRHSGVKD | 0.1 | 0.5 | 75 |
| IGGYSEDRHSGVKDY | 0.1 | 0.6 | 76 |
| GGYSEDRHSGVKDYV | 0.1 | 0.2 | 77 |
| GYSEDRHSGVKDYVV | 0.1 | 0.3 | 78 |
| YSEDRHSGVKDYVVV | 0.1 | 0.4 | 79 |
| SEDRHSGVKDYVVVH | 0.1 | 0.4 | 80 |
| EDRHSGVKDYVVVHG | 0.1 | 0.3 | 81 |
| DRHSGVKDYVVVHGY | 0.1 | 0.5 | 82 |
| RHSGVKDYVVVHGYF | 0.1 | 0.4 | 83 |
| HSGVKDYVVVHGYFT | 0.1 | 0.6 | 84 |
| SGVKDYVVVHGYFTE | 0.1 | 0.7 | 85 |
| GVKDYVVVHGYFTEV | 0.1 | 0.7 | 86 |
| VKDYVVVHGYFTEVY | 0.1 | 0.6 | 709 |
| KDYVVVHGYFTEVYY | 0.1 | 0.5 | 710 |
| DYVVVHGYFTEVYYQ | 0.1 | 0.6 | 711 |
| YVVVHGYFTEVYYQL | 0.1 | 0.5 | 712 |
| VVVHGYFTEVYYQLE | 0.1 | 0.6 | 713 |
| VVHGYFTEVYYQLES | 0.1 | 0.7 | 714 |
| VHGYFTEVYYQLEST | 0.1 | 0.6 | 715 |
| HGYFTEVYYQLESTQ | 0.1 | 0.5 | 716 |
| GYFTEVYYQLESTQI | 0.1 | 0.5 | 717 |
| YFTEVYYQLESTQIT | 0.1 | 0.4 | 718 |
| FTEVYYQLESTQITT | 0.1 | 0.5 | 719 |
| TEVYYQLESTQITTD | 0.1 | 0.4 | 720 |
| EVYYQLESTQITTDT | 0.1 | 0.4 | 721 |
| VYYQLESTQITTDTG | 0.1 | 0.5 | 722 |
| YYQLESTQITTDTGI | 0.1 | 0.6 | 723 |
| YQLESTQITTDTGIE | 0.1 | 0.4 | 724 |
| QLESTQITTDTGIEN | 0.1 | 0.6 | 725 |
| LESTQITTDTGIENA | 0.1 | 0.5 | 726 |
| ESTQITTDTGIENAT | 0.1 | 0.5 | 727 |
| STQITTDTGIENATF | 0.1 | 0.7 | 728 |
| TQITTDTGIENATFF | 0.1 | 0.6 | 729 |
| QITTDTGIENATFFI | 0.1 | 1.5 | 730 |
| ITTDTGIENATFFIF | 0.1 | 0.6 | 731 |
| TTDTGIENATFFIFN | 0.1 | 0.5 | 732 |
| TDTGIENATFFIFNK | 0.1 | 0.6 | 733 |
| DTGIENATFFIFNKL | 0.1 | 0.6 | 734 |
| TGIENATFFIFNKLV | 0.1 | 0.6 | 735 |
| GIENATFFIFNKLVK | 0.1 | 0.6 | 736 |
| IENATFFIFNKLVKD | 0.1 | 0.5 | 737 |
| ENATFFIFNKLVKDP | 0.1 | 0.5 | 738 |
| NATFFIFNKLVKDPP | 0.1 | 0.3 | 739 |
| ATFFIFNKLVKDPPN | 0.1 | 0.6 | 87 |
| TFFIFNKLVKDPPNV | 0.1 | 0.5 | 88 |
| FFIFNKLVKDPPNVQ | 0.1 | 0.5 | 89 |
| FIFNKLVKDPPNVQI | 0.1 | 0.7 | 90 |
| IFNKLVKDPPNVQIH | 0.1 | 0.6 | 91 |
| FNKLVKDPPNVQIHT | 0.1 | 0.7 | 92 |
| NKLVKDPPNVQIHTI | 0.1 | 0.6 | 93 |
| KLVKDPPNVQIHTID | 0.1 | 0.4 | 94 |
| LVKDPPNVQIHTIDG | 0.1 | 0.5 | 95 |
| VKDPPNVQIHTIDGS | 0.1 | 0.5 | 96 |
| KDPPNVQIHTIDGSS | 0.1 | 0.9 | 97 |
| DPPNVQIHTIDGSSG | 0.1 | 0.4 | 740 |
| PPNVQIHTIDGSSGV | 0.1 | 0.5 | 741 |
| PNVQIHTIDGSSGVA | 0.1 | 0.4 | 742 |
| NVQIHTIDGSSGVAN | 0.1 | 0.3 | 743 |
| VQIHTIDGSSGVANP | 0.1 | 0.4 | 744 |
| QIHTIDGSSGVANPA | 0.1 | 0.3 | 745 |
| IHTIDGSSGVANPAM | 0.1 | 0.5 | 746 |
| HTIDGSSGVANPAMD | 0.1 | 0.4 | 747 |
| TIDGSSGVANPAMDP | 0.1 | 0.4 | 748 |
| IDGSSGVANPAMDPI | 0.1 | 0.5 | 749 |
| DGSSGVANPAMDPIY | 0.1 | 0.6 | 98 |
| GSSGVANPAMDPIYD | 0.1 | 0.5 | 99 |
| SSGVANPAMDPIYDE | 0.1 | 0.6 | 100 |
| SGVANPAMDPIYDEP | 1.1 | 0.6 | 101 |
| GVANPAMDPIYDEPT | 0.1 | 0.6 | 102 |
| VANPAMDPIYDEPTT | 0.1 | 0.5 | 103 |
| ANPAMDPIYDEPTTT | 0.1 | 0.4 | 104 |
| NPAMDPIYDEPTTTT | 0.1 | 0.3 | 105 |
| PAMDPIYDEPTTTTS | 0.1 | 0.4 | 106 |
| AMDPIYDEPTTTTSV | 0.1 | 0.4 | 107 |
| MDPIYDEPTTTTSVP | 0.1 | 0.3 | 108 |
| DPIYDEPTTTTSVPL | 0.1 | 0.5 | 109 |

TABLE 26
Binding of a rabbit serum to linear and looped/cyclic
peptides of protein X2 of SARS-CoV Urbani.

| | Rabbit serum | Rabbit serum | |
|---|---|---|---|
| Peptide | linear | looped | |
| sequence | peptides | peptides | SEQ ID NO |
| MMPTTLFAGTHITMT | 0.6 | 0.6 | 110 |
| MPTTLFAGTHITMTT | 0.7 | 0.3 | 111 |
| PTTLFAGTHITMTTV | 0.8 | 0.4 | 112 |
| TTLFAGTHITMTTVY | 0.6 | 0.3 | 113 |
| TLFAGTHITMTTVYH | 0.7 | 0.5 | 114 |
| LFAGTHITMTTVYHI | 0.6 | 0.5 | 115 |
| FAGTHITMTTVYHIT | 0.6 | 0.4 | 116 |
| AGTHITMTTVYHITV | 0.7 | 0.5 | 117 |
| GTHITMTTVYHITVS | 2.1 | 0.4 | 118 |
| THITMTTVYHITVSQ | 0.7 | 0.4 | 750 |
| HITMTTVYHITVSQI | 0.7 | 0.4 | 751 |
| ITMTTVYHITVSQIQ | 0.3 | 0.3 | 752 |
| TMTTVYHITVSQIQL | 0.7 | 0.4 | 753 |
| MTTVYHITVSQIQLS | 0.7 | 0.4 | 754 |
| TTVYHITVSQIQLSL | 0.7 | 0.4 | 755 |
| TVYHITVSQIQLSLL | 0.7 | 0.3 | 756 |
| VYHITVSQIQLSLLK | 0.9 | 0.4 | 757 |
| YHITVSQIQLSLLKV | 0.8 | 0.3 | 758 |
| HITVSQIQLSLLKVT | 0.7 | 0.4 | 759 |
| ITVSQIQLSLLKVTA | 0.7 | 0.4 | 760 |
| TVSQIQLSLLKVTAF | 0.6 | 0.5 | 761 |
| VSQIQLSLLKVTAFQ | 0.6 | 0.4 | 762 |
| SQIQLSLLKVTAFQH | 0.7 | 0.5 | 763 |
| QIQLSLLKVTAFQHQ | 0.6 | 0.5 | 764 |
| IQLSLLKVTAFQHQN | 0.6 | 0.5 | 765 |
| QLSLLKVTAFQHQNS | 0.6 | 0.5 | 766 |
| LSLLKVTAFQHQNSK | 0.4 | 0.3 | 767 |
| SLLKVTAFQHQNSKK | 0.1 | 0.3 | 768 |
| LLKVTAFQHQNSKKT | 0.6 | 0.3 | 769 |
| LKVTAFQHQNSKKTT | 0.6 | 0.3 | 770 |
| KVTAFQHQNSKKTTK | 0.6 | 0.2 | 771 |
| VTAFQHQNSKKTTKL | 0.6 | 0.4 | 772 |
| 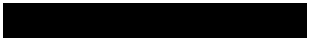 | 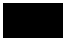 | 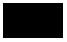 | 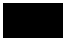 |
| 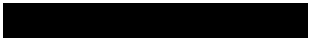 | 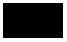 | 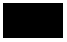 | 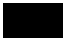 |
| 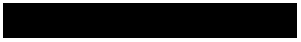 | 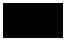 | 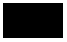 | 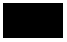 |
| 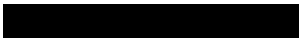 | 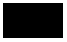 | 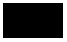 | 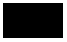 |
| 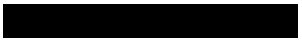 | 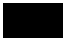 | 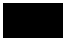 | 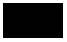 |
| 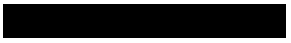 | 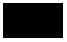 | 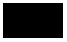 | 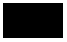 |
| 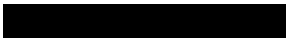 | 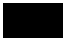 | 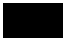 | 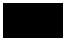 |
| 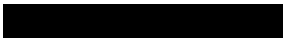 | 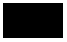 | 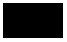 | 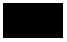 |
| 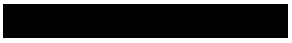 | 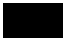 | 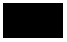 | 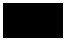 |
| 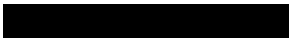 | 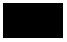 | 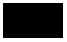 | 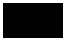 |
| 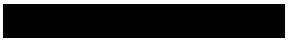 | 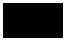 | 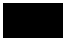 | 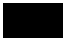 |
| TKLVVILRIGTQVLK | 0.3 | 0.6 | 128 |
| KLVVILRIGTQVLKT | 0.5 | 0.5 | 129 |
| LVVILRIGTQVLKTM | 0.4 | 0.6 | 773 |
| VVILRIGTQVLKTMS | 0.4 | 0.4 | 774 |
| VILRIGTQVLKTMSL | 0.3 | 0.5 | 775 |
| ILRIGTQVLKTMSLY | 0.3 | 0.5 | 776 |
| LRIGTQVLKTMSLYM | 0.4 | 0.4 | 130 |
| RIGTQVLKTMSLYMA | 0.4 | 0.5 | 131 |
| IGTQVLKTMSLYMAI | 0.1 | 0.4 | 132 |
| GTQVLKTMSLYMAIS | 0.2 | 0.4 | 133 |
| TQVLKTMSLYMAISP | 0.1 | 0.4 | 134 |
| QVLKTMSLYMAISPK | 0.1 | 0.5 | 135 |
| VLKTMSLYMAISPKF | 0.3 | 0.5 | 136 |
| LKTMSLYMAISPKFT | 0.1 | 0.4 | 137 |
| KTMSLYMAISPKFTT | 0.3 | 0.8 | 138 |
| TMSLYMAISPKFTTS | 0.2 | 0.5 | 777 |
| MSLYMAISPKFTTSL | 0.3 | 0.5 | 778 |
| SLYMAISPKFTTSLS | 0.2 | 0.3 | 779 |
| LYMAISPKFTTSLSL | 0.3 | 0.5 | 780 |
| YMAISPKFTTSLSLH | 0.3 | 0.5 | 781 |
| MAISPKFTTSLSLHK | 0.3 | 0.6 | 782 |
| AISPKFTTSLSLHKL | 0.2 | 0.4 | 783 |
| ISPKFTTSLSLHKLL | 0.3 | 0.4 | 784 |
| SPKFTTSLSLHKLLQ | 0.3 | 0.5 | 785 |
| PKFTTSLSLHKLLQT | 0.2 | 0.4 | 786 |
| KFTTSLSLHKLLQTL | 0.2 | 0.4 | 787 |
| FTTSLSLHKLLQTLV | 0.1 | 0.9 | 788 |
| TTSLSLHKLLQTLVL | 0.1 | 0.4 | 789 |
| TSLSLHKLLQTLVLK | 0.2 | 0.5 | 790 |
| SLSLHKLLQTLVLKM | 0.1 | 0.4 | 791 |
| LSLHKLLQTLVLKML | 0.3 | 0.5 | 792 |
| SLHKLLQTLVLKMLH | 0.3 | 0.5 | 793 |
| LHKLLQTLVLKMLHS | 0.2 | 0.4 | 794 |
| HKLLQTLVLKMLHSS | 0.2 | 0.4 | 795 |
| KLLQTLVLKMLHSSS | 0.2 | 0.4 | 796 |
| LLQTLVLKMLHSSSL | 0.2 | 0.4 | 797 |
| LQTLVLKMLHSSSLT | 0.3 | 0.3 | 798 |
| QTLVLKMLHSSSLTS | 0.3 | 0.4 | 799 |
| TLVLKMLHSSSLTSL | 0.2 | 0.4 | 800 |
| LVLKMLHSSSLTSLL | 0.3 | 0.4 | 801 |
| VLKMLHSSSLTSLLK | 0.2 | 0.3 | 802 |
| LKMLHSSSLTSLLKT | 0.2 | 0.4 | 803 |
| KMLHSSSLTSLLKTH | 0.2 | 0.2 | 804 |
| MLHSSSLTSLLKTHR | 0.2 | 0.5 | 805 |
| LHSSSLTSLLKTHRM | 0.2 | 0.4 | 806 |
| HSSSLTSLLKTHRMC | 0.2 | 0.5 | 807 |
| SSSLTSLLKTHRMCK | 0.3 | 0.2 | 808 |
| SSLTSLLKTHRMCKY | 0.3 | 0.5 | 809 |
| SLTSLLKTHRMCKYT | 0.1 | 0.3 | 810 |
| LTSLLKTHRMCKYTQ | 0.3 | 0.4 | 811 |
| TSLLKTHRMCKYTQS | 0.4 | 0.4 | 812 |
| SLLKTHRMCKYTQST | 0.3 | 0.3 | 813 |
| LLKTHRMCKYTQSTA | 0.5 | 0.3 | 814 |
| LKTHRMCKYTQSTAL | 0.4 | 0.5 | 815 |
| KTHRMCKYTQSTALQ | 0.3 | 0.3 | 816 |
| THRMCKYTQSTALQE | 0.3 | 0.3 | 817 |
| HRMCKYTQSTALQEL | 0.3 | 0.4 | 818 |
| RMCKYTQSTALQELL | 0.4 | 0.4 | 819 |
| MCKYTQSTALQELLI | 0.2 | 0.4 | 820 |
| CKYTQSTALQELLIQ | 0.2 | 0.2 | 821 |
| KYTQSTALQELLIQQ | 0.4 | 0.4 | 822 |
| YTQSTALQELLIQQW | 0.3 | 0.3 | 823 |
| TQSTALQELLIQQWI | 0.3 | 0.4 | 824 |
| QSTALQELLIQQWIQ | 0.2 | 0.4 | 825 |
| STALQELLIQQWIQF | 0.3 | 0.4 | 826 |
| TALQELLIQQWIQFM | 0.4 | 0.4 | 827 |
| ALQELLIQQWIQFMM | 0.2 | 0.4 | 828 |
| LQELLIQQWIQFMMS | 0.3 | 0.3 | 829 |
| QELLIQQWIQFMMSR | 0.3 | 0.4 | 830 |
| ELLIQQWIQFMMSRR | 0.4 | 0.4 | 831 |
| LLIQQWIQFMMSRRR | 0.5 | 0.6 | 832 |
| LIQQWIQFMMSRRRL | 0.3 | 1.2 | 833 |
| IQQWIQFMMSRRRLL | 0.4 | 1.0 | 834 |
| QQWIQFMMSRRRLLA | 0.8 | 1.5 | 835 |
| QWIQFMMSRRRLLAC | 1.0 | 2.0 | 836 |
| WIQFMMSRRRLLACL | 0.5 | 1.3 | 837 |
| IQFMMSRRRLLACLC | 0.5 | 1.1 | 838 |
| QFMMSRRRLLACLCK | 0.5 | 1.9 | 839 |
| FMMSRRRLLACLCKH | 0.4 | 0.6 | 840 |
| MMSRRRLLACLCKHK | 0.4 | 0.3 | 139 |
| MSRRRLLACLCKHKK | 0.2 | 0.3 | 140 |
| SRRRLLACLCKHKKV | 0.3 | 0.4 | 141 |
| RRRLLACLCKHKKVS | 0.5 | 0.2 | 142 |
| RRLLACLCKHKKVST | 0.6 | 0.3 | 143 |
| RLLACLCKHKKVSTN | 0.3 | 0.4 | 144 |
| LLACLCKHKKVSTNL | 0.4 | 0.4 | 145 |
| LACLCKHKKVSTNLC | 0.3 | 0.3 | 146 |
| ACLCKHKKVSTNLCT | 0.3 | 0.3 | 147 |
| CLCKHKKVSTNLCTH | 0.4 | 0.4 | 148 |
| LCKHKKVSTNLCTHS | 0.3 | 0.3 | 149 |
| CKHKKVSTNLCTHSF | 0.3 | 0.5 | 150 |
| KHKKVSTNLCTHSFR | 0.4 | 0.0 | 151 |
| HKKVSTNLCTHSFRK | 0.4 | 0.1 | 152 |
| KKVSTNLCTHSFRKK | 0.3 | 0.1 | 153 |
| KVSTNLCTHSFRKKQ | 0.3 | 0.1 | 154 |
| VSTNLCTHSFRKKQV | 0.3 | 0.2 | 155 |
| STNLCTHSFRKKQVR | 0.7 | 0.2 | 156 |

TABLE 27

Binding of a rabbit serum to linear and
looped/cyclic peptides of protein E of
SARS-CoV Urbani.

| | Rabbit | | |
|---|---|---|---|
| | serum | Rabbit serum | |
| Peptide | linear | looped | |
| sequence | peptides | peptides | SEQ ID NO |
| MYSFVSEETGTLIVN | 0.5 | 0.2 | 841 |
| YSFVSEETGTLIVNS | 0.2 | 0.4 | 842 |
| SFVSEETGTLIVNSV | 0.5 | 0.1 | 843 |
| VSEETGTLIVNSVLL | 0.4 | 0.1 | 844 |
| FVSEETGTLIVNSVL | 0.3 | 0.1 | 845 |
| SEETGTLIVNSVLLF | 0.7 | 0.0 | 846 |
| EETGTLIVNSVLLFL | 0.5 | 0.0 | 847 |
| ETGTLIVNSVLLFLA | 0.3 | 0.0 | 848 |
| TGTLIVNSVLLFLAF | 0.5 | 0.0 | 849 |
| GTLIVNSVLLFLAFV | 0.6 | 0.0 | 850 |
| TLIVNSVLLFLAFVV | 0.1 | 0.3 | 851 |
| LIVNSVLLFLAFVVF | 0.5 | 0.3 | 852 |
| IVNSVLLFLAFVVFL | 0.5 | 0.5 | 853 |
| VNSVLLFLAFVVFLL | 0.2 | 0.4 | 854 |
| NSVLLFLAFVVFLLV | 0.6 | 0.6 | 855 |
| SVLLFLAFVVFLLVT | 0.6 | 0.2 | 856 |
| VLLFLAFVVFLLVTL | 0.5 | 0.5 | 857 |
| LLFLAFVVFLLVTLA | 0.6 | 0.4 | 858 |
| LFLAFVVFLLVTLAI | 0.5 | 0.3 | 859 |
| FLAFVVFLLVTLAIL | 0.0 | 0.2 | 860 |
| LAFVVFLLVTLAILT | 0.5 | 0.2 | 861 |
| AFVVFLLVTLAILTA | 0.7 | 0.1 | 862 |
| FVVFLLVTLAILTAL | 0.2 | 0.2 | 863 |
| VVFLLVTLAILTALR | 0.5 | 0.2 | 864 |
| VFLLVTLAILTALRL | 0.4 | 0.3 | 865 |
| FLLVTLAILTALRLC | 0.1 | 0.0 | 866 |
| LLVTLAILTALRLCA | 0.5 | 0.8 | 867 |
| LVTLAILTALRLCAY | 0.4 | 0.3 | 868 |
| VTLAILTALRLCAYC | 0.1 | 0.4 | 869 |
| TLAILTALRLCAYCC | 0.7 | 0.4 | 870 |
| LAILTALRLCAYCCN | 0.6 | 0.6 | 871 |
| AILTALRLCAYCCNI | 0.2 | 0.6 | 872 |
| ILTALRLCAYCCNIV | 0.6 | 0.6 | 873 |
| LTALRLCAYCCNIVN | 0.6 | 0.4 | 874 |
| TALRLCAYCCNIVNV | 0.2 | 0.4 | 875 |
| ALRLCAYCCNIVNVS | 0.7 | 0.4 | 876 |
| LRLCAYCCNIVNVSL | 0.6 | 0.2 | 877 |
| RLCAYCCNIVNVSLV | 0.4 | 0.2 | 878 |
| LCAYCCNIVNVSLVK | 0.7 | 0.4 | 157 |
| CAYCCNIVNVSLVKP | 0.6 | 0.2 | 158 |
| AYCCNIVNVSLVKPT | 0.3 | 0.2 | 159 |
| YCCNIVNVSLVKPTV | 0.7 | 0.3 | 160 |
| CCNIVNVSLVKPTVY | 0.6 | 0.0 | 161 |
| CNIVNVSLVKPTVYV | 0.1 | 0.5 | 162 |
| NIVNVSLVKPTVYVY | 0.5 | 0.6 | 163 |
| IVNVSLVKPTVYVYS | 0.5 | 0.5 | 164 |
| VNVSLVKPTVYVYSR | 0.4 | 0.6 | 165 |
| NVSLVKPTVYVYSRV | 0.5 | 0.3 | 166 |
| VSLVKPTVYVYSRVK | 1.5 | 2.0 | 167 |
| SLVKPTVYVYSRVKN | 0.3 | 0.6 | 168 |
| LVKPTVYVYSRVKNL | 0.6 | 0.9 | 169 |
| VKPTVYVYSRVKNLN | 0.5 | 0.7 | 170 |
| KPTVYVYSRVKNLNS | 0.6 | 0.9 | 171 |
| PTVYVYSRVKNLNSS | 0.7 | 0.7 | 172 |
| TVYVYSRVKNLNSSE | 0.8 | 0.3 | 173 |
| VYVYSRVKNLNSSEG | 0.3 | 0.4 | 174 |
| YVYSRVKNLNSSEGV | 1.2 | 0.5 | 175 |
| VYSRVKNLNSSEGVP | 0.7 | 0.0 | 176 |
| YSRVKNLNSSEGVPD | 0.3 | 0.0 | 177 |
| SRVKNLNSSEGVPDL | 0.7 | 0.0 | 178 |
| RVKNLNSSEGVPDLL | 0.8 | 0.6 | 179 |
| VKNLNSSEGVPDLLV | 0.3 | 0.6 | 180 |

TABLE 28
Binding of a rabbit serum to linear and looped/cyclic
peptides of protein M of SARS-CoV Urbani.

| | Rabbit serum | Rabbit serum | |
|---|---|---|---|
| Peptide | linear | looped | SEQ ID |
| sequence | peptides | peptides | NO |
| 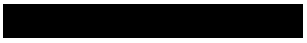 | 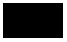 | 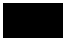 | 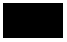 |
| 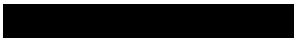 | 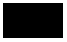 | 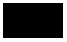 | 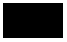 |
| 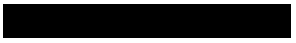 | 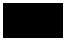 | 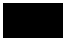 | 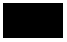 |
| 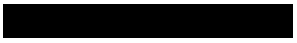 | 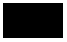 | 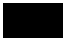 | 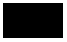 |
| 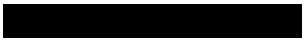 | 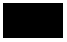 | 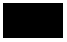 | 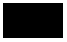 |
| 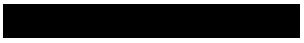 | 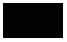 | 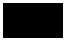 | 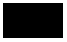 |
| 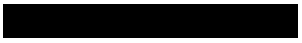 | 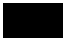 | 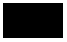 | 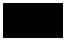 |
| 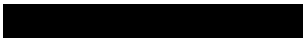 | 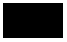 | 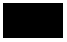 | 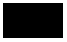 |
| 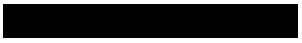 | 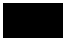 | 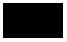 | 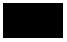 |
| EELKQLLEQWNLVIG | 0.4 | 0.4 | 190 |
| ELKQLLEQWNLVIGF | 0.0 | 0.2 | 879 |
| LKQLLEQWNLVIGFL | 0.2 | 0.1 | 880 |
| KQLLEQWNLVIGFLP | 0.0 | 0.1 | 881 |
| QLLEQWNLVIGFLFL | 0.2 | 0.0 | 882 |
| LLEQWNLVIGFLFLA | 0.6 | 0.0 | 883 |
| LEQWNLVIGFLFLAW | 0.2 | 0.4 | 884 |
| EQWNLVIGFLFLAWI | 0.2 | 0.4 | 885 |
| QWNLVIGFLFLAWIM | 0.3 | 0.3 | 886 |
| WNLVIGFLFLAWIML | 0.2 | 0.3 | 887 |
| NLVIGFLFLAWIMLL | 0.2 | 0.2 | 888 |
| LVIGFLFLAWIMLLQ | 0.1 | 0.3 | 889 |
| VIGFLFLAWIMLLQF | 0.3 | 0.3 | 890 |
| IGFLFLAWIMLLQFA | 0.2 | 0.3 | 891 |
| GFLFLAWIMLLQFAY | 0.3 | 0.4 | 892 |
| FLFLAWIMLLQFAYS | 0.2 | 0.3 | 893 |
| LFLAWIMLLQFAYSN | 0.7 | 0.3 | 894 |
| FLAWIMLLQFAYSNR | 0.3 | 0.5 | 895 |
| LAWIMLLQFAYSNRN | 0.8 | 0.1 | 896 |
| AWIMLLQFAYSNRNR | 0.3 | 0.4 | 897 |
| WIMLLQFAYSNRNRF | 0.8 | 0.3 | 898 |
| IMLLQFAYSNRNRFL | 0.2 | 0.5 | 899 |
| MLLQFAYSNRNRFLY | 0.5 | 0.0 | 900 |
| LLQFAYSNRNRFLYI | 0.2 | 0.4 | 901 |
| LQFAYSNRNRFLYII | 0.6 | 0.4 | 902 |
| QFAYSNRNRFLYIIK | 0.4 | 1.2 | 191 |
| FAYSNRNRFLYIIKL | 0.7 | 0.6 | 192 |
| AYSNRNRFLYIIKLV | 0.3 | 0.5 | 193 |
| YSNRNRFLYIIKLVF | 0.4 | 0.5 | 194 |
| SNRNRFLYIIKLVFL | 0.5 | 0.6 | 195 |
| NRNRFLYIIKLVFLW | 0.5 | 0.4 | 196 |
| RNRFLYIIKLVFLWL | 0.4 | 0.5 | 197 |
| NRFLYIIKLVFLWLL | 0.4 | 0.3 | 198 |
| RFLYIIKLVFLWLLW | 0.2 | 0.3 | 199 |
| FLYIIKLVFLWLLWP | 0.4 | 0.4 | 200 |
| LYIIKLVFLWLLWPV | 0.1 | 0.3 | 903 |
| YIIKLVFLWLLWPVT | 0.4 | 0.2 | 904 |
| IIKLVFLWLLWPVTL | 0.1 | 0.0 | 905 |
| IKLVFLWLLWPVTLA | 0.3 | 0.0 | 906 |
| KLVFLWLLWPVTLAC | 0.1 | 0.0 | 907 |
| LVFLWLLWPVTLACF | 0.3 | 0.3 | 908 |
| VFLWLLWPVTLACFV | 0.3 | 0.3 | 909 |
| FLWLLWPVTLACFVL | 0.3 | 0.4 | 910 |
| LWLLWPVTLACFVLA | 0.1 | 0.4 | 911 |
| WLLWPVTLACFVLAA | 0.3 | 0.3 | 912 |
| LLWPVTLACFVLAAV | 0.2 | 0.3 | 913 |
| LWPVTLACFVLAAVY | 0.4 | 0.4 | 914 |
| WPVTLACFVLAAVYR | 0.2 | 0.4 | 915 |
| PVTLACFVLAAVYRI | 0.4 | 0.3 | 916 |
| VTLACFVLAAVYRIN | 0.2 | 0.3 | 917 |
| TLACFVLAAVYRINW | 0.5 | 0.1 | 918 |
| LACFVLAAVYRINWV | 0.3 | 0.2 | 919 |
| ACFVLAAVYRINWVT | 0.4 | 0.3 | 920 |
| CFVLAAVYRINWVTG | 0.2 | 0.1 | 921 |
| FVLAAVYRINWVTGG | 0.5 | 0.0 | 922 |
| VLAAVYRINWVTGGI | 0.3 | 0.0 | 923 |
| LAAVYRINWVTGGIA | 0.4 | 0.0 | 924 |
| AAVYRINWVTGGIAI | 0.4 | 0.4 | 925 |
| AVYRINWVTGGIAIA | 0.4 | 0.5 | 926 |
| VYRINWVTGGIAIAM | 0.3 | 0.4 | 927 |
| YRINWVTGGIAIAMA | 0.4 | 0.3 | 928 |
| RINWVTGGIAIAMAC | 0.2 | 0.4 | 929 |
| INWVTGGIAIAMACI | 0.5 | 0.4 | 201 |
| NWVTGGIAIAMACIV | 0.2 | 0.3 | 202 |
| WVTGGIAIAMACIVG | 0.4 | 0.2 | 203 |
| VTGGIAIAMACIVGL | 0.3 | 0.4 | 204 |
| TGGIAIAMACIVGLM | 0.5 | 0.3 | 205 |
| GGIAIAMACIVGLMW | 0.2 | 0.3 | 206 |
| GIAIAMACIVGLMWL | 0.3 | 0.1 | 207 |
| IAIAMACIVGLMWLS | 0.1 | 0.3 | 208 |
| AIAMACIVGLMWLSY | 0.4 | 0.0 | 930 |
| IANACIVGLMWLSYF | 0.1 | 0.0 | 931 |
| AMACIVGLMWLSYFV | 0.4 | 0.1 | 932 |
| MACIVGLMWLSYFVA | 0.2 | 0.0 | 933 |
| ACIVGLMWLSYFVAS | 0.3 | 0.8 | 934 |
| CIVGLMWLSYFVASF | 0.1 | 0.3 | 935 |
| IVGLMWLSYFVASFR | 0.3 | 0.5 | 936 |
| VGLMWLSYFVASFRL | 0.0 | 0.4 | 937 |
| GLMWLSYFVASFRLF | 0.2 | 0.2 | 938 |
| LMWLSYFVASFRLFA | 0.0 | 0.3 | 209 |
| MWLSYFVASFRLFAR | 0.4 | 0.5 | 210 |
| WLSYFVASFRLFART | 0.2 | 0.4 | 211 |
| LSYFVASFRLFARTR | 0.4 | 0.6 | 212 |
| SYFVASFRLFARTRS | 0.2 | 0.4 | 213 |
| YFVASFRLFARTRSM | 0.6 | 0.8 | 214 |
| FVASFRLFARTRSMW | 0.2 | 0.3 | 215 |
| VASFRLFARTRSMWS | 0.8 | 0.4 | 216 |
| ASFRLFARTRSMWSF | 0.3 | 0.2 | 939 |
| SFRLFARTRSMWSFN | 0.8 | 0.2 | 940 |
| FRLFARTRSMWSFNP | 0.2 | 0.2 | 941 |
| RLFARTRSMWSFNPE | 0.4 | 0.0 | 942 |
| LFARTRSMWSFNPET | 0.1 | 0.3 | 943 |
| FARTRSMWSFNPETN | 0.4 | 0.3 | 944 |
| ARTRSMWSFNPETNI | 0.2 | 0.3 | 945 |
| RTRSMWSFNPETNIL | 0.6 | 0.4 | 946 |
| TRSMWSFNPETNILL | 0.1 | 0.3 | 947 |
| RSMWSFNPETNILLN | 0.4 | 0.3 | 948 |
| SMWSFNPETNILLNV | 0.2 | 0.4 | 949 |
| MWSFNPETNILLNVP | 0.4 | 0.2 | 950 |
| WSFNPETNILLNVPL | 0.3 | 0.3 | 951 |
| SFNPETNILLNVPLR | 0.5 | 0.4 | 952 |
| FNPETNILLNVPLRG | 0.0 | 0.4 | 953 |
| NPETNILLNVPLRGT | 0.4 | 0.2 | 954 |
| PETNILLNVPLRGTI | 0.1 | 0.3 | 955 |
| ETNILLNVPLRGTIV | 0.5 | 0.0 | 956 |
| TNILLNVPLRGTIVT | 0.2 | 0.1 | 957 |
| NILLNVPLRGTIVTR | 0.5 | 0.4 | 217 |
| ILLNVPLRGTIVTRP | 0.0 | 0.2 | 218 |
| LLNVPLRGTIVTRPL | 0.4 | 0.0 | 219 |
| LNVPLRGTIVTRPLM | 0.2 | 0.4 | 220 |
| NVPLRGTIVTRPLME | 0.3 | 0.5 | 221 |
| VPLRGTIVTRPLMES | 0.1 | 0.6 | 222 |
| PLRGTIVTRPLMESE | 0.4 | 0.4 | 223 |
| LRGTIVTRPLMESEL | 0.0 | 0.5 | 224 |
| RGTIVTRPLMESELV | 0.3 | 0.3 | 225 |
| GTIVTRPLMESELVI | 0.1 | 0.5 | 226 |
| TIVTRPLMESELVIG | 0.3 | 0.3 | 227 |
| IVTRPLMESELVIGA | 0.1 | 0.4 | 229 |
| VTRPLMESELVIGAV | 0.4 | 0.2 | 230 |
| TRPLMESELVIGAVI | 0.2 | 0.3 | 231 |
| RPLMESELVIGAVII | 0.4 | 0.2 | 232 |
| PLMESELVIGAVIIR | 0.2 | 0.1 | 958 |
| LMESELVIGAVIIRG | 0.4 | 0.2 | 959 |
| MESELVIGAVIIRGH | 0.2 | 0.2 | 960 |
| ESELVIGAVIIRGHL | 0.4 | 0.1 | 961 |
| SELVIGAVIIRGHLR | 0.2 | 0.2 | 962 |
| ELVIGAVIIRGHLRM | 0.8 | 0.1 | 963 |
| LVIGAVIIRGHLRMA | 0.2 | 1.4 | 964 |
| VIGAVIIRGHLRMAG | 1.5 | 0.6 | 233 |
| IGAVIIRGHLRMAGH | 0.3 | 0.8 | 234 |
| GAVIIRGHLRMAGHP | 0.8 | 0.5 | 235 |
| AVIIRGHLRMAGHPL | 0.3 | 0.6 | 236 |
| VIIRGHLRMAGHPLG | 0.4 | 0.6 | 237 |
| IIRGHLRMAGHPLGR | 0.6 | 2.0 | 238 |
| IRGHLRMAGHPLGRC | 1.2 | 0.4 | 239 |
| RGHLRMAGHPLGRCD | 0.0 | 0.3 | 240 |
| GHLRMAGHPLGRCDI | 0.4 | 0.7 | 241 |
| HLRMAGHPLGRCDIK | 0.0 | 0.5 | 242 |
| LRMAGHPLGRCDIKD | 0.3 | 0.1 | 243 |
| RMAGHPLGRCDIKDL | 0.1 | 0.6 | 244 |
| MAGHPLGRCDIKDLP | 0.4 | 0.3 | 245 |
| AGHPLGRCDIKDLPK | 0.1 | 0.1 | 246 |
| GHPLGRCDTKDLPKE | 0.3 | 0.0 | 247 |
| HPLGRCDIKDLPKEI | 0.2 | 1.1 | 248 |
| PLGRCDIKDLPKEIT | 0.3 | 0.4 | 249 |
| LGRCDIKDLPKEITV | 0.1 | 0.6 | 250 |
| GRCDIKDLPKEITVA | 0.0 | 0.1 | 251 |
| RCDIKDLPKEITVAT | 0.0 | 0.3 | 965 |
| CDIKDLPKEITVATS | 0.4 | 0.6 | 966 |
| DIKDLPKEITVATSR | 0.2 | 0.3 | 967 |
| IKDLPKEITVATSRT | 0.3 | 0.2 | 968 |
| KDLPKEITVATSRTL | 0.2 | 0.3 | 969 |
| DLPKEITVATSRTLS | 0.1 | 0.3 | 970 |
| LPKEITVATSRTLSY | 0.2 | 0.2 | 971 |
| PKEITVATSRTLSYY | 0.5 | 0.2 | 972 |
| KEITVATSRTLSYYK | 0.4 | 0.5 | 973 |
| EITVATSRTLSYYKL | 0.7 | 0.1 | 974 |
| ITVATSRTLSYYKLG | 0.2 | 0.4 | 975 |
| TVATSRTLSYYKLGA | 0.8 | 0.2 | 976 |
| VATSRTLSYYKLGAS | 0.3 | 0.7 | 977 |
| ATSRTLSYYKLGASQ | 0.6 | 0.4 | 978 |
| TSRTLSYYKLGASQR | 0.3 | 1.1 | 979 |
| SRTLSYYKLGASQRV | 0.6 | 0.6 | 980 |
| RTLSYYKLGASQRVG | 0.5 | 1.0 | 981 |
| TLSYYKLGASQRVGT | 0.4 | 0.5 | 252 |
| LSYYKLGASQRVGTD | 0.2 | 0.3 | 253 |
| SYYKLGASQRVGTDS | 0.4 | 0.3 | 254 |
| YYKLGASQRVGTDSG | 0.1 | 0.0 | 255 |
| YKLGASQRVGTDSGF | 0.4 | 0.2 | 256 |
| KLGASQRVGTDSGFA | 0.1 | 0.1 | 257 |
| LGASQRVGTDSGFAA | 0.3 | 0.1 | 258 |
| GASQRVGTDSGFAAY | 0.1 | 0.1 | 259 |
| ASQRVGTDSGFAAYN | 0.4 | 0.1 | 260 |
| SQRVGTDSGFAAYNR | 0.2 | 0.1 | 982 |
| QRVGTDSGFAAYNRY | 0.4 | 0.0 | 983 |
| RVGTDSGFAAYNRYR | 0.0 | 0.3 | 984 |
| VGTDSGFAAYNRYRI | 0.4 | 0.0 | 985 |
| GTDSGFAAYNRYRIG | 0.2 | 0.4 | 986 |
| TDSGFAAYNRYRIGN | 0.0 | 0.5 | 987 |
| DSGFAAYNRYRIGNY | 0.2 | 0.3 | 988 |
| SGFAAYNRYRIGNYK | 1.2 | 1.6 | 989 |
| GFAAYNRYRIGNYKL | 0.2 | 0.5 | 990 |
| FAAYNRYRIGNYKLN | 0.3 | 0.6 | 991 |
| AAYNRYRIGNYKLNT | 0.4 | 0.6 | 992 |
| AYNRYRIGNYKLNTD | 0.3 | 0.3 | 993 |
| YNRYRIGNYKLNTDH | 0.3 | 0.5 | 994 |
| NRYRIGNYKLNTDHA | 0.2 | 0.3 | 995 |
| RYRIGNYKLNTDHAG | 0.1 | 0.2 | 996 |
| YRIGNYKLNTDHAGS | 0.6 | 0.1 | 997 |
| RIGNYKLNTDHAGSN | 0.1 | 0.2 | 998 |
| IGNYKLNTDHAGSND | 0.5 | 0.0 | 261 |
| GNYKLNTDHAGSNDN | 0.0 | 0.2 | 262 |
| NYKLNTDHAGSNDNI | 0.5 | 0.0 | 263 |
| YKLNTDHAGSNDNIA | 0.1 | 0.1 | 264 |
| KLNTDHAGSNDNIAL | 0.4 | 0.3 | 265 |
| LNTDHAGSNDNIALL | 0.0 | 0.3 | 266 |
| NTDHAGSNDNIALLV | 0.4 | 0.4 | 267 |
| TDHAGSNDNIALLVQ | 0.1 | 0.2 | 268 |

TABLE 29

Binding of a rabbit serum to linear and
looped/cyclic peptides of protein X3 of
SARS-CoV Urbani.

| | | | |
|---|---|---|---|
| | Rabbit | | |
| | serum | Rabbit serum | |
| Peptide | linear | looped | |
| sequence | peptides | peptides | SEQ ID NO |

| | | | |
|---|---|---|---|
| MFHLVDFQVTIAEIL | 0.3 | 0.4 | 999 |
| FHLVDFQVTIAEILI | 0.3 | 0.5 | 1000 |
| HLVDFQVTIAEILII | 0.3 | 0.3 | 1001 |
| LVDFQVTIAEILIII | 0.3 | 0.3 | 1002 |
| VDFQVTIAEILIIIM | 0.3 | 0.3 | 1003 |
| DFQVTIAEILIIIMR | 0.3 | 0.3 | 1004 |
| FQVTIAEILIIIMRT | 0.2 | 0.3 | 1005 |
| QVTIAEILIIIMRTF | 0.3 | 0.6 | 1006 |
| VTIAEILIIIMRTFR | 0.2 | 0.4 | 1007 |
| TIAEILIIIMRTFRI | 0.2 | 0.0 | 1008 |
| IAEILIIIMRTFRIA | 0.3 | 0.2 | 1009 |
| AEILIIIMRTFRIAI | 0.3 | 0.0 | 269 |
| EILIIIMRTFRIAIW | 0.5 | 0.3 | 270 |
| ILIIIMRTFRIAIWN | 0.5 | 0.6 | 271 |
| LIIIMRTFRIAIWNL | 0.5 | 0.3 | 272 |
| IIIMRTFRIAIWNLD | 0.4 | 0.5 | 273 |
| IIMRTFRIAIWNLDV | 0.3 | 0.5 | 274 |
| IMRTFRIAIWNLDVI | 0.4 | 0.6 | 275 |
| MRTFRIAIWNLDVII | 0.3 | 0.4 | 276 |
| RTFRIAIWNLDVIIS | 0.3 | 0.4 | 277 |
| TFRIAIWNLDVIISS | 0.3 | 0.4 | 1010 |
| FRIAIWNLDVIISSI | 0.3 | 0.2 | 1011 |
| RIAIWNLDVIISSIV | 0.3 | 0.4 | 1012 |
| IAIWNLDVIISSIVR | 0.3 | 0.3 | 1013 |
| AIWNLDVIISSIVRQ | 0.3 | 0.4 | 1014 |
| IWNLDVIISSIVRQL | 0.2 | 0.4 | 1015 |
| WNLDVIISSIVRQLF | 0.1 | 0.2 | 1016 |
| NLDVIISSIVRQLFK | 0.3 | 0.2 | 1017 |
| LDVIISSIVRQLFKP | 0.2 | 0.0 | 1018 |
| DVIISSIVRQLFKPL | 0.5 | 0.2 | 1019 |
| VIISSIVRQLFKPLT | 0.5 | 0.3 | 278 |
| IISSIVRQLFKPLTK | 0.6 | 0.4 | 279 |
| ISSIVRQLFKPLTKK | 0.3 | 0.5 | 280 |
| SSIVRQLFKPLTKKN | 0.4 | 0.5 | 281 |
| SIVRQLFKPLTKKNY | 0.3 | 0.4 | 282 |
| IVRQLFKPLTKKNYS | 0.4 | 0.6 | 283 |
| VRQLFKPLTKKNYSE | 0.4 | 2.2 | 284 |
| RQLFKPLTKKNYSEL | 0.3 | 0.5 | 285 |
| QLFKPLTKKNYSELD | 0.3 | 0.3 | 286 |
| LFKPLTKKNYSELDD | 0.3 | 0.4 | 287 |
| FKPLTKKNYSELDDE | 0.5 | 0.5 | 288 |
| KPLTKKNYSELDDEE | 0.4 | 0.5 | 289 |
| PLTKKNYSELDDEEP | 0.2 | 0.4 | 290 |
| LTKKNYSELDDEEPM | 0.2 | 0.4 | 291 |
| TKKNYSELDDEEPME | 0.2 | 0.1 | 292 |
| KKNYSELDDEEPMEL | 0.1 | 0.0 | 293 |
| KNYSELDDEEPMELD | 0.3 | 0.2 | 294 |
| NYSELDDEEPMELDY | 0.4 | 0.3 | 295 |
| YSELDDEEPMELDYP | 0.3 | 0.3 | 296 |

TABLE 30
Binding of a rabbit serum to linear and looped/cyclic
peptides of protein X4 of SARS-CoV Urbani.

| | Rabbit serum | Rabbit Serum | |
|---|---|---|---|
| Peptide | linear | looped | |
| sequence | peptides | peptides | SEQ ID NO |
| MKIILFLTLIVFTSC | 0.7 | 0.4 | 1020 |
| KIILFLTLIVFTSCE | 0.7 | 0.9 | 1021 |
| IILFLTLIVFTSCEL | 0.8 | 0.5 | 1022 |
| ILFLTLIVFTSCELY | 0.7 | 0.2 | 1023 |
| LFLTLIVFTSCELYH | 0.7 | 0.4 | 1024 |
| FLTLIVFTSCELYHY | 0.4 | 0.5 | 1025 |
| LTLIVFTSCELYHYQ | 0.5 | 0.3 | 1026 |
| TLIVFTSCELYHYQE | 0.5 | 0.8 | 1027 |
| LIVFTSCELYHYQEC | 0.5 | 0.5 | 1028 |
| IVFTSCELYHYQECV | 0.4 | 0.4 | 1029 |
| VFTSCELYHYQECVR | 0.5 | 0.5 | 1030 |
| FTSCELYHYQECVRG | 0.4 | 0.3 | 1031 |
| TSCELYHYQECVRGT | 0.4 | 0.2 | 1032 |
| SCELYHYQECVRGTT | 0.4 | 0.0 | 1033 |
| CELYHYQECVRGTTV | 0.4 | 0.2 | 1034 |
| ELYHYQECVRGTTVL | 0.7 | 0.7 | 297 |
| LYHYQECVRGTTVLL | 0.6 | 0.3 | 298 |
| YHYQECVRGTTVLLK | 1.7 | 0.6 | 299 |
| HYQECVRGTTVLLKE | 0.5 | 0.5 | 300 |
| YQECVRGTTVLLKEP | 0.5 | 0.5 | 301 |
| QECVRGTTVLLKEPC | 0.6 | 0.5 | 302 |
| ECVRGTTVLLKEPCP | 0.6 | 0.4 | 303 |
| CVRGTTVLLKEPCPS | 0.5 | 0.5 | 304 |
| VRGTTVLLKEPCPSG | 0.4 | 0.4 | 305 |
| RGTTVLLKEPCPSGT | 0.4 | 0.5 | 306 |
| GTTVLLKEPCPSGTY | 0.4 | 0.4 | 307 |
| TTVLLKEPCPSGTYE | 0.4 | 0.5 | 308 |
| TVLLKEPCPSGTYEG | 0.2 | 0.2 | 309 |
| VLLKEPCPSGTYEGN | 0.4 | 0.3 | 1035 |
| LLKEPCPSGTYEGNS | 0.3 | 0.1 | 1036 |
| LKEPCPSGTYEGNSP | 0.4 | 0.0 | 1037 |
| KEPCPSGTYEGNSPF | 0.4 | 0.3 | 1038 |
| EPCPSGTYEGNSPFH | 0.6 | 0.4 | 1039 |
| PCPSGTYEGNSPFHP | 0.6 | 0.4 | 1040 |
| CPSGTYEGNSPFNPL | 0.5 | 0.7 | 310 |
| PSGTYEGNSPFHPLA | 0.5 | 0.5 | 311 |
| SGTYEGNSPFHPLAD | 0.6 | 0.6 | 312 |
| GTYEGNSPFHPLADN | 0.6 | 0.5 | 313 |
| TYEGNSPFHPLADNK | 0.7 | 0.4 | 314 |
| YEGNSPFHPLADNKF | 0.6 | 0.5 | 315 |
| EGNSPFHPLADNKFA | 0.7 | 0.7 | 316 |
| GNSPFHPLADNKFAL | 0.5 | 1.0 | 317 |
| NSPFHPLADNKFALT | 0.5 | 0.7 | 318 |
| SPFHPLADNKFALTC | 0.4 | 0.5 | 319 |
| PFHPLADNKFALTCT | 0.4 | 0.4 | 320 |
| FHPLADNKFALTCTS | 0.4 | 0.2 | 321 |
| HPLADNKFALTCTST | 0.5 | 0.1 | 322 |
| PLADNKFALTCTSTH | 1.1 | 0.0 | 323 |
| LADNKFALTCTSTHF | 0.5 | 0.5 | 324 |
| ADNKFALTCTSTHFA | 0.7 | 0.2 | 325 |
| DNKFALTCTSTHFAF | 0.7 | 0.6 | 326 |
| NKFALTCTSTHFAFA | 0.5 | 0.5 | 1041 |
| KFALTCTSTHFAFAC | 0.5 | 0.6 | 1042 |
| FALTCTSTHFAFACA | 0.6 | 0.4 | 1043 |
| ALTCTSTHFAFACAD | 0.6 | 0.6 | 1044 |
| LTCTSTHFAFACADG | 0.5 | 0.3 | 1045 |
| TCTSTHFAFACADGT | 0.5 | 0.7 | 1046 |
| CTSTHFAFACADGTR | 0.4 | 0.6 | 1047 |
| TSTHFAFACADGTRH | 0.5 | 0.7 | 1048 |
| STHFAFACADGTRHT | 0.4 | 0.4 | 1049 |
| THFAFACADGTRHTY | 0.4 | 0.5 | 1050 |
| HFAFACADGTRHTYQ | 0.4 | 0.1 | 1051 |
| FAFACADGTRHTYQL | 0.5 | 0.1 | 1052 |
| AFACADGTRHTYQLR | 0.5 | 0.1 | 1053 |
| 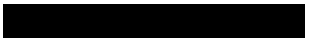 | 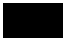 | 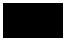 | 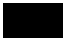 |
| 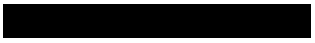 | 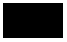 | 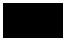 | 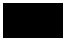 |
| 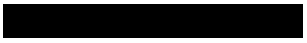 | 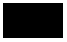 | 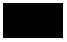 | 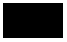 |
| 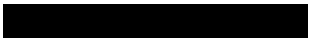 | 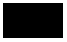 | 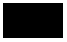 | 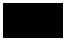 |
| 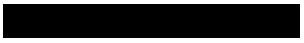 | 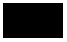 | 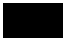 | 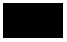 |
| 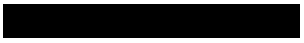 | 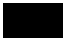 | 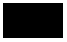 | 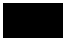 |
| 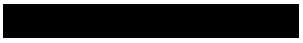 | 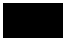 | 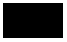 | 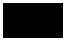 |
| 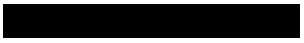 | 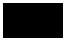 | 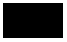 | 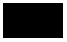 |
| 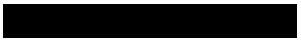 | 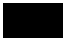 | 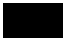 | 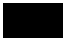 |
| 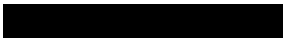 | 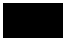 | 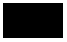 | 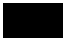 |
| 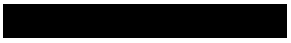 | 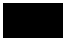 | 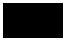 | 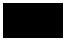 |
| 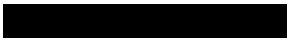 | 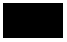 | 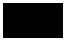 | 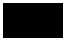 |
| 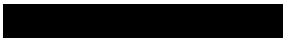 | 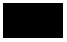 | 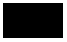 | 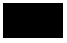 |
| 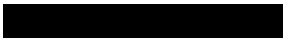 | 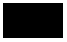 | 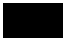 | 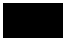 |
| ARSVSPKLFIRQEEV | 0.3 | 0.2 | 1054 |
| RSVSPKLFIRQEEVQ | 0.4 | 0.4 | 1055 |
| SVSPKLFIRQEEVQQ | 0.4 | 0.3 | 1056 |
| VSPKLFIRQEEVQQE | 0.4 | 0.3 | 1057 |
| SPKLFIRQEEVQQEL | 0.5 | 0.0 | 1058 |
| PKLFIRQEEVQQELY | 0.4 | 0.4 | 1059 |
| KLFIRQEEVQQELYS | 0.5 | 0.5 | 1060 |
| LFIRQEEVQQELYSP | 0.4 | 0.4 | 1061 |
| FIRQEEVQQELYSPL | 0.5 | 0.5 | 327 |
| IRQEEVQQELYSPLF | 0.4 | 0.4 | 328 |
| RQEEVQQELYSPLFL | 0.5 | 0.6 | 329 |
| QEEVQQELYSPLFLI | 0.4 | 0.4 | 330 |
| EEVQQELYSPLFLIV | 0.4 | 0.5 | 331 |
| EVQQELYSPLFLIVA | 0.5 | 0.5 | 332 |
| VQQELYSPLFLTVAA | 0.5 | 0.2 | 333 |
| QQELYSPLFLIVAAL | 0.4 | 0.4 | 1062 |
| QELYSPLFLIVAALV | 0.4 | 0.5 | 1063 |
| ELYSPLFLIVAALVF | 0.4 | 0.5 | 1064 |
| LYSPLFLIVAALVFL | 0.4 | 0.3 | 1065 |
| YSPLFLIVAALVFLI | 0.3 | 0.5 | 1066 |
| SPLFLIVAALVFLIL | 0.5 | 0.8 | 1067 |
| PLFLIVAALVFLILC | 0.4 | 0.0 | 1068 |
| LFLIVAALVFLILCF | 0.3 | 0.4 | 1069 |
| FLIVAALVFLILCFT | 0.4 | 0.3 | 1070 |
| LIVAALVFLILCFTI | 0.5 | 0.4 | 1071 |
| IVAALVFLILCFTIK | 0.4 | 0.6 | 1072 |
| VAALVFLILCFTIKR | 0.5 | 0.6 | 1073 |
| AALVFLILCFTIKRK | 0.8 | 0.6 | 1074 |
| ALVFLILCFTIKRKT | 0.6 | 0.6 | 1075 |
| LVFLILCFTIKRKTE | 0.5 | 0.6 | 1076 |

TABLE 31

Binding of a rabbit serum to linear and
looped/cyclic peptides of protein X5 of
SARS-CoV Urbani.

| | | | |
|---|---|---|---|
| | Rabbit | | |
| | serum | Rabbit serum | |
| Peptide | linear | looped | |
| sequence | peptides | peptides | SEQ ID NO |

| | | | |
|---|---|---|---|
| MCLKILVRYNTRGNT | 0.7 | 0.5 | 1077 |
| CLKILVRYNTRGNTY | 1.1 | 0.2 | 1078 |
| LKILVRYNTRGNTYS | 0.9 | 0.1 | 1079 |
| KILVRYNTRGNTYST | 0.7 | 0.4 | 1080 |
| ILVRYNTRGNTYSTA | 0.9 | 0.8 | 1081 |
| LVRYNTRGNTYSTAW | 0.7 | 0.3 | 1082 |
| VRYNTRGNTYSTAWL | 0.7 | 1.2 | 1083 |
| RYNTRGNTYSTAWLC | 0.7 | 0.0 | 1084 |
| YNTRGNTYSTAWLCA | 0.7 | 0.0 | 1085 |
| NTRGNTYSTAWLCAL | 0.8 | 0.5 | 1086 |
| TRGNTYSTAWLCALG | 0.7 | 0.0 | 1087 |
| RGNTYSTAWLCALGK | 1.3 | 0.8 | 1088 |
| GNTYSTAWLCALGKV | 0.9 | 0.6 | 1089 |
| NTYSTAWLCALGKVL | 0.6 | 0.5 | 1090 |
| TYSTAWLCALGKVLP | 0.6 | 0.7 | 1091 |
| YSTAWLCALGKVLPF | 0.7 | 1.0 | 1092 |
| STAWLCALGKVLPFH | 0.5 | 0.7 | 1093 |
| TAWLCALGKVLPFHR | 0.7 | 0.8 | 1094 |
| AWLCALGKVLPFHRW | 0.8 | 0.6 | 1095 |
| WLCALGKVLPFHRWH | 0.6 | 0.8 | 1096 |
| LCALGKVLPFHRWHT | 0.7 | 0.7 | 1097 |
| CALGKVLPFHRWHTM | 0.6 | 1.0 | 1098 |
| ALGKVLPFHRWHTMV | 0.6 | 0.1 | 1099 |
| LGKVLPFHRWHTMVQ | 0.5 | 0.3 | 1100 |
| GKVLPFHRWHTMVQT | 0.0 | 0.3 | 1101 |
| KVLPFHRWHTMVQTC | 0.6 | 0.5 | 1102 |
| VLPFHRWHTMVQTCT | 0.6 | 0.0 | 1103 |
| LPFHRWHTMVQTCTP | 0.5 | 0.3 | 1104 |
| PFHRWHTMVQTCTPN | 0.5 | 0.4 | 1105 |
| FHRWHTMVQTCTPNV | 0.8 | 0.4 | 1106 |
| HRWHTMVQTCTPNVT | 0.7 | 0.3 | 1107 |
| RWHTMVQTCTPNVTI | 0.5 | 0.6 | 334 |
| WHTMVQTCTPNVTIN | 0.5 | 0.0 | 335 |
| HTMVQTCTPNVTINC | 0.4 | 0.2 | 336 |
| TMVQTCTPNVTINCQ | 0.5 | 0.4 | 337 |
| MVQTCTPNVTINCQD | 0.5 | 0.2 | 338 |
| VQTCTPNVTINCQDP | 0.5 | 0.4 | 1108 |
| QTCTPNVTINCQDPA | 0.3 | 0.0 | 1109 |
| TCTPNVTINCQDPAG | 0.5 | 0.0 | 1110 |
| CTPNVTINCQDPAGG | 0.4 | 0.0 | 1111 |
| TPNVTINCQDPAGGA | 0.0 | 0.1 | 1112 |
| PNVTINCQDPAGGAL | 0.6 | 0.2 | 339 |
| NVTINCQDPAGGALI | 0.6 | 0.5 | 340 |
| VTINCQDPAGGALIA | 0.5 | 0.0 | 341 |
| TINCQDPAGGALIAR | 0.6 | 0.7 | 342 |
| INCQDPAGGALIARC | 0.5 | 0.6 | 343 |
| NCQDPAGGALIARCW | 0.5 | 0.5 | 344 |
| CQDPAGGALIARCWY | 0.5 | 0.6 | 345 |
| QDPAGGALIARCWYL | 0.5 | 0.9 | 346 |
| DPAGGALIARCWYLH | 0.3 | 0.5 | 1113 |
| PAGGALIARCWYLHE | 0.5 | 0.5 | 1114 |
| AGGALIARCWYLHEG | 0.5 | 0.4 | 1115 |
| GGALIARCWYLHEGH | 0.5 | 0.5 | 1116 |
| GALIARCWYLHEGHQ | 0.6 | 0.0 | 1117 |
| ALIARCWYLHEGHQT | 0.6 | 0.0 | 1118 |
| LIARCWYLHEGHQTA | 0.8 | 0.0 | 1119 |
| IARCWYLHEGHQTAA | 0.7 | 0.3 | 347 |
| ARCWYLHEGHQTAAF | 0.3 | 0.6 | 348 |
| RCWYLHEGHQTAAFR | 0.9 | 0.4 | 349 |
| CWYLHEGHQTAAFRD | 0.3 | 0.4 | 350 |
| WYLHEGHQTAAFRDV | 0.3 | 0.0 | 351 |
| YLHEGHQTAAFRDVL | 0.2 | 0.6 | 352 |
| LHEGHQTAAFRDVLV | 0.5 | 0.7 | 353 |
| HEGHQTAAFRDVLVV | 0.2 | 0.8 | 354 |
| EGHQTAAFRDVLVVL | 0.3 | 0.6 | 355 |
| GHQTAAFRDVLVVLN | 0.1 | 0.5 | 356 |
| HQTAAFRDVLVVLNK | 0.4 | 0.8 | 357 |
| QTAAFRDVLVVLNKR | 0.4 | 0.5 | 1120 |
| TAAFRDVLVVLNKRT | 0.3 | 0.4 | 1121 |
| AAFRDVLVVLNKRTN | 0.5 | 0.6 | 1122 |

TABLE 32
Binding of a rabbit serum to linear and looped/cyclic
peptides of protein N of SARS-CoV Urbani.

| | Rabbit serum | Rabbit serum | |
|---|---|---|---|
| Peptide | linear | looped | SEQ ID |
| sequence | peptides | peptides | NO |
| MSDNGPQSNQRSAPR | 0.1 | 0.4 | 1123 |
| SDNGPQSNQRSAPRI | 0.1 | 0.2 | 1124 |
| DNGPQSNQRSAPRIT | 0.1 | 0.3 | 1125 |
| 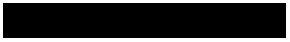 | 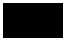 | 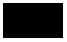 | 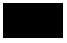 |
| 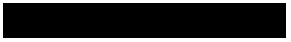 | 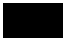 | 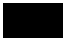 | 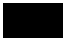 |
| 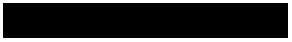 | 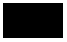 | 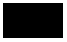 | 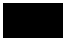 |
| 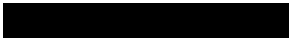 | 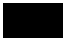 | 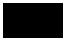 | 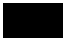 |
| SNQRSAPRITFGGPT | 0.4 | 0.9 | 596 |
| NQRSAPRITFGGPTD | 0.3 | 0.4 | 597 |
| QRSAPRITFGGPTDS | 0.3 | 1.2 | 598 |
| RSAPRITFGGPTDST | 0.3 | 0.7 | 599 |
| SAPRITFGGPTDSTD | 0.2 | 0.5 | 600 |
| APRITFGGPTDSTDN | 0.2 | 0.6 | 601 |
| PRITFGGPTDSTDNN | 0.3 | 0.4 | 602 |
| RITFGGPTDSTDNNQ | 0.3 | 0.1 | 603 |
| ITFGGPTDSTDNNQN | 0.1 | 0.3 | 604 |
| TFGGPTDSTDNNQNG | 0.1 | 0.1 | 1126 |
| FGGPTDSTDNNQNGG | 0.0 | 0.1 | 1127 |
| GGPTDSTDNNQNGGR | 0.1 | 0.1 | 1128 |
| GPTDSTDNNQNGGRN | 0.1 | 0.4 | 1129 |
| PTDSTDNNQNGGRNG | 0.1 | 0.1 | 1130 |
| TDSTDNNQNGGRNGA | 0.1 | 0.2 | 1131 |
| DSTDNNQNGGRNGAR | 0.1 | 0.5 | 1132 |
| STDNNQNGGRNGARP | 0.1 | 0.2 | 1133 |
| TDNNQNGGRNGARPK | 0.2 | 0.4 | 1134 |
| DNNQNGGRNGARPKQ | 0.1 | 0.4 | 1135 |
| NNQNGGRNGARPKQR | 0.3 | 0.7 | 1136 |
| NQNGGRNGARPKQRR | 0.5 | 1.1 | 1137 |
| QNGGRNGARPKQRRP | 0.2 | 0.4 | 1138 |
| NGGRNGARPKQRRPQ | 0.1 | 1.1 | 1139 |
| GGRNGARPKQRRPQG | 0.2 | 0.8 | 1140 |
| GRNGARPKQRRPQGL | 0.2 | 0.7 | 1141 |
| RNGARPKQRRPQGLP | 0.1 | 0.2 | 1142 |
| NGARPKQRRPQGLPN | 0.1 | 0.3 | 1143 |
| GARPKQRRPQGLPNN | 0.1 | 0.2 | 1144 |
| ARPKQRRPQGLPNNT | 0.2 | 0.4 | 1145 |
| RPKQRRPQGLPNNTA | 0.4 | 0.4 | 1146 |
| PKQRRPQGLPNNTAS | 0.2 | 0.2 | 1147 |
| KQRRPQGLPNNTASW | 0.1 | 0.6 | 1148 |
| QRRPQGLPNNTASWF | 0.1 | 0.6 | 1149 |
| RRPQGLPNNTASWFT | 0.1 | 0.4 | 1150 |
| RPQGLPNNTASWFTA | 0.1 | 0.5 | 1151 |
| PQGLPNNTASWFTAL | 0.1 | 0.6 | 1152 |
| QGLPNNTASWFTALT | 0.1 | 0.6 | 1153 |
| GLPNNTASWFTALTQ | 0.1 | 0.4 | 1154 |
| LPNNTASWFTALTQH | 0.1 | 0.6 | 1155 |
| PNNTASWFTALTQHG | 0.1 | 0.4 | 1156 |
| NNTASWFTALTQHGK | 0.1 | 0.3 | 1157 |
| NTASWFTALTQHGKE | 0.1 | 0.1 | 1158 |
| TASWFTALTQHGKEE | 0.1 | 0.7 | 1159 |
| ASWFTALTQHGKEEL | 0.1 | 0.1 | 1160 |
| SWFTALTQHGKEELR | 0.1 | 0.0 | 1161 |
| WFTALTQHGKEELRF | 0.1 | 0.3 | 1162 |
| FTALTQHGKEELRFP | 0.2 | 0.1 | 1163 |
| TALTQHGKEELRFPR | 0.1 | 0.4 | 1164 |
| ALTQHGKEELRFPRG | 0.2 | 0.3 | 1165 |
| LTQHGKEELRFPRGQ | 0.1 | 0.4 | 1166 |
| TQHGKEELRFPRGQG | 0.1 | 0.3 | 1167 |
| QHGKEELRFPRGQGV | 0.2 | 0.5 | 1168 |
| HGKEELRFPRGQGVP | 0.1 | 0.3 | 1169 |
| GKEELRFPRGQGVPI | 0.1 | 0.6 | 1170 |
| KEELRFPRGQGVPIN | 0.1 | 0.6 | 1171 |
| EELRFPRGQGVPINT | 0.1 | 0.6 | 1172 |
| ELRFPRGQGVPINTN | 0.1 | 0.4 | 1173 |
| LRFPRGQGVPINTNS | 0.2 | 0.6 | 1174 |
| RFPRGQGVPINTNSG | 0.1 | 0.5 | 1175 |
| FPRGQGVPINTNSGP | 0.2 | 0.2 | 1176 |
| PRGQGVPINTNSGPD | 0.1 | 0.0 | 1177 |
| RGQGVPINTNSGPDD | 0.1 | 0.0 | 1178 |
| GQGVPINTNSGPDDQ | 0.1 | 0.0 | 1179 |
| QGVPINTNSGPDDQI | 0.2 | 0.8 | 1180 |
| GVPINTNSGPDDQIG | 0.1 | 0.2 | 1181 |
| VPINTNSGPDDQIGY | 0.1 | 0.3 | 1182 |
| PINTNSGPDDQIGYY | 0.1 | 0.4 | 1183 |
| INTNSGPDDQIGYYR | 0.2 | 0.5 | 1184 |
| NTNSGPDDQIGYYRR | 0.1 | 0.6 | 1185 |
| TNSGPDDQIGYYRRA | 0.2 | 0.5 | 1186 |
| NSGPDDQIGYYRRAT | 0.1 | 0.5 | 1187 |
| 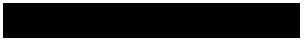 | 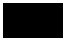 | 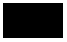 | 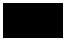 |
| 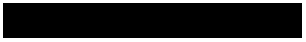 | 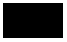 | 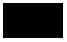 | 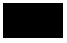 |
| 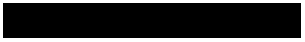 | 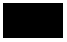 | 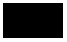 | 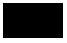 |
| 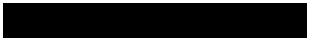 | 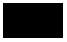 | 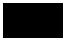 | 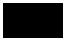 |
| 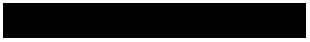 | 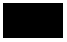 | 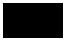 | 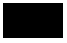 |
| 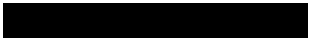 | 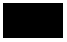 | 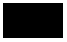 | 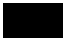 |
| 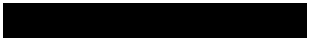 | 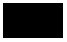 | 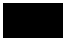 | 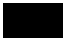 |
| 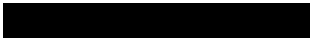 | 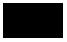 | 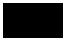 | 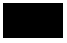 |
| YYRRATRRVRGGDGK | 0.2 | 0.0 | 1188 |
| YRRATRRVRGGDGKM | 0.2 | 0.0 | 1189 |
| RRATRRVRGGDGKMK | 0.6 | 0.1 | 1190 |
| RATRRVRGGDGKMKE | 0.2 | 0.2 | 1191 |
| ATRRVRGGDGKMKEL | 0.3 | 0.3 | 1192 |
| TRRVRGGDGKMKELS | 0.2 | 0.3 | 1193 |
| RRVRGGDGKMKELSP | 0.2 | 0.4 | 1194 |
| RVRGGDGKMKELSPR | 0.2 | 0.6 | 1195 |
| VRGGDGKMKELSPRW | 0.2 | 0.4 | 1196 |
| RGGDGKMKELSPRWY | 0.1 | 0.4 | 1197 |
| GGDGKMKELSPRWYF | 0.1 | 0.5 | 1198 |
| GDGKMKELSPRWYFY | 0.2 | 0.6 | 1199 |
| DGKMKELSPRWYFYY | 0.1 | 0.6 | 1200 |
| GKMKELSPRWYFYYL | 0.1 | 0.7 | 1201 |
| KMKELSPRWYFYYLG | 0.1 | 0.6 | 1202 |
| MKELSPRWYFYYLGT | 0.1 | 0.4 | 1203 |
| KELSPRWYFYYLGTG | 0.0 | 0.4 | 1204 |
| ELSPRWYFYYLGTGP | 0.1 | 0.4 | 1205 |
| LSPRWYFYYLGTGPE | 0.2 | 1.1 | 1206 |
| SPRWYFYYLGTGPEA | 0.2 | 0.6 | 1207 |
| PRWYFYYLGTGPEAS | 0.2 | 0.6 | 1208 |
| RWYFYYLGTGPEASL | 0.2 | 0.6 | 1209 |
| WYPYYLGTGPEASLP | 0.1 | 0.3 | 1210 |
| YFYYLGTGPEASLPY | 0.1 | 0.6 | 1211 |
| FYYLGTGPEASLPYG | 0.1 | 0.6 | 1212 |
| YYLGTGPEASLPYGA | 0.1 | 0.5 | 1213 |
| YLGTGPEASLPYGAN | 0.1 | 0.6 | 1214 |
| LGTGPEASLPYGANK | 0.2 | 0.4 | 1215 |
| GTGPEASLPYGANKE | 0.1 | 0.3 | 1216 |
| TGPEASLPYGANKEG | 0.1 | 0.5 | 1217 |
| GPEASLPYGANKEGI | 0.1 | 0.3 | 1218 |
| PEASLPYGANKEGIV | 0.1 | 0.2 | 1219 |
| EASLPYGANKEGIVW | 0.1 | 0.3 | 1220 |
| ASLPYGANKEGIVWV | 0.2 | 0.2 | 1221 |
| SLPYGANKEGIVWVA | 0.2 | 0.3 | 1222 |
| LPYGANKEGIVWVAT | 0.2 | 0.6 | 1223 |
| PYGANKEGIVWVATE | 0.2 | 0.2 | 1224 |
| YGANKEGIVWVATEG | 0.1 | 0.5 | 1225 |
| GANKEGIVWVATEGA | 0.2 | 0.5 | 1226 |
| ANKEGIVWVATEGAL | 0.1 | 0.3 | 1227 |
| NKEGIVWVATEGALN | 0.1 | 0.2 | 1228 |
| KEGIVWVATEGALNT | 0.1 | 0.4 | 1229 |
| EGIVWVATEGALNTP | 0.1 | 0.4 | 1230 |
| GIVWVATEGALNTPK | 0.2 | 0.5 | 1231 |
| IVWVATEGALNTPKD | 0.1 | 0.2 | 1232 |
| VWVATEGALNTPKDH | 0.1 | 0.4 | 1233 |
| WVATEGALNTPKDHI | 0.1 | 0.2 | 1234 |
| VATEGALNTPKDHIG | 0.2 | 0.0 | 1235 |
| ATEGALNTPKDHIGT | 0.1 | 0.1 | 1236 |
| TEGALNTPKDHIGTR | 0.2 | 0.0 | 1237 |
| EGALNTPKDHIGTRN | 0.1 | 0.0 | 1238 |
| GALNTPKDHIGTRNP | 0.2 | 0.0 | 1239 |
| ALNTPKDHIGTRNPN | 0.2 | 0.2 | 1240 |
| LNTPKDHIGTRNPNN | 0.2 | 0.0 | 1241 |
| NTPKDHIGTRNPNNN | 0.1 | 0.1 | 1242 |
| TPKDHIGTRNPNNNA | 0.1 | 0.4 | 1243 |
| PKDHIGTRNPNNNAA | 0.2 | 0.2 | 1244 |
| KDHIGTRNPNNNAAT | 0.1 | 0.2 | 1245 |
| DHIGTRNPNNNAATV | 0.1 | 0.5 | 1246 |
| HIGTRNPNNNAATVL | 0.1 | 0.7 | 1247 |
| IGTRNPNNNAATVLQ | 0.1 | 0.4 | 1248 |
| GTRNPNNNAATVLQL | 0.1 | 0.6 | 1249 |
| TRNPNNNAATVLQLP | 0.1 | 0.5 | 1250 |
| RNPNNNAATVLQLPQ | 0.1 | 0.8 | 1251 |
| NPNNNAATVLQLPQG | 0.1 | 0.3 | 1252 |
| PNNNAATVLQLPQGT | 0.0 | 0.2 | 1253 |
| NNNAATVLQLPQGTT | 0.1 | 0.4 | 1254 |
| NNAATVLQLPQGTTL | 0.2 | 0.4 | 358 |
| NAATVLQLPQGTTLP | 0.2 | 0.0 | 359 |
| AATVLQLPQGTTLPK | 0.3 | 1.2 | 360 |
| ATVLQLPQGTTLPKG | 0.2 | 0.3 | 361 |
| TVLQLPQGTTLPKGF | 0.3 | 1.2 | 362 |
| VLQLPQGTTLPKGFY | 0.2 | 0.4 | 363 |
| LQLPQGTTLPKGFYA | 0.2 | 1.0 | 364 |
| QLPQGTTLPKGFYAE | 0.2 | 0.4 | 365 |
| LPQGTTLPKGFYAEG | 0.1 | 0.7 | 366 |
| PQGTTLPKGFYAEGS | 0.1 | 0.5 | 367 |
| QGTTLPKGFYAEGSR | 0.1 | 0.6 | 368 |
| GTTLPKGFYAEGSRG | 0.2 | 0.3 | 369 |
| TTLPKGFYAEGSRGG | 0.2 | 0.4 | 370 |
| TLPKGFYAEGSRGGS | 0.5 | 0.3 | 371 |
| LPKGFYAEGSRGGSQ | 0.1 | 0.1 | 1255 |
| PKGFYAEGSRGGSQA | 0.1 | 0.1 | 1256 |
| KGFYAEGSRGGSQAS | 0.1 | 0.1 | 1257 |
| GFYAEGSRGGSQASS | 0.1 | 0.0 | 1258 |
| FYAEGSRGGSQASSR | 0.3 | 0.4 | 1259 |
| YAEGSRGGSQASSRS | 0.2 | 0.0 | 1260 |
| AEGSRGGSQASSRSS | 0.2 | 0.6 | 1261 |
| EGSRGGSQASSRSSS | 0.2 | 0.7 | 1262 |
| GSRGGSQASSRSSSR | 0.3 | 0.6 | 1263 |
| SRGGSQASSRSSSRS | 0.2 | 0.5 | 1264 |
| RGGSQASSRSSSRSR | 0.3 | 0.7 | 1265 |
| GGSQASSRSSSRSRG | 0.2 | 0.5 | 1266 |
| GSQASSRSSSRSRGN | 0.2 | 0.7 | 1267 |
| SQASSRSSSRSRGNS | 0.1 | 0.6 | 1268 |
| QASSRSSSRSRGNSR | 0.3 | 1.1 | 1269 |
| ASSRSSSRSRGNSRN | 0.3 | 0.7 | 1270 |
| SSRSSSRSRGNSRNS | 0.2 | 0.7 | 1271 |
| SRSSSRSRGNSRNST | 0.1 | 0.3 | 1272 |
| RSSSRSRGNSRNSTP | 0.1 | 0.2 | 1273 |
| SSSRSRGNSRNSTPG | 0.1 | 0.4 | 1274 |
| SSRSRGNSRNSTPGS | 0.1 | 0.0 | 1275 |
| SRSRGNSRNSTPGSS | 0.3 | 0.7 | 1276 |
| RSRGNSRNSTPGSSR | 0.4 | 1.1 | 1277 |
| SRGNSRNSTPGSSRG | 0.2 | 0.2 | 1278 |
| RGNSRNSTPGSSRGN | 0.2 | 0.7 | 1279 |
| GNSRNSTPGSSRGNS | 0.2 | 0.8 | 1280 |
| NSRNSTPGSSRGNSP | 0.2 | 0.5 | 1281 |
| SRNSTPGSSRGNSPA | 0.1 | 0.6 | 1282 |
| 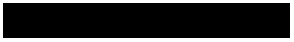 | 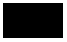 | 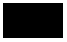 | 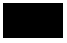 |
| 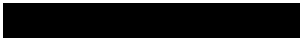 | 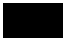 | 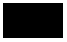 | 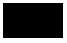 |
| 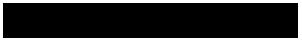 | 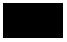 | 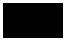 | 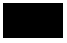 |
| 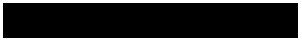 | 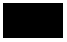 | 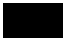 | 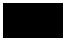 |
| 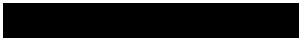 | 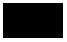 | 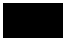 | 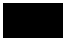 |
| 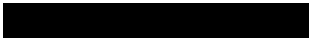 | 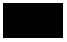 | 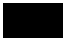 | 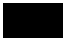 |
| SSRGNSPARMASGGG | 0.2 | 0.0 | 1283 |
| SRGNSPARMASGGGE | 0.1 | 0.2 | 1284 |
| RGNSPARMASGGGET | 0.1 | 0.2 | 1285 |
| GNSPARMASGGGETA | 0.1 | 0.0 | 1286 |
| NSPARMASGGGETAL | 0.3 | 0.3 | 372 |
| SPARMASGGGETALA | 0.2 | 0.0 | 373 |
| PARMASGGGETALAL | 0.2 | 0.7 | 374 |
| ARMASGGGETALALL | 0.2 | 0.0 | 375 |
| RMASGGGETALALLL | 0.2 | 0.4 | 376 |
| MASGGGETALALLLL | 0.2 | 1.1 | 377 |
| ASGGGETALALLLLD | 0.1 | 0.4 | 378 |
| SGGGETALALLLLDR | 0.2 | 0.7 | 1287 |
| GGGETALALLLLDRL | 0.1 | 0.7 | 1288 |
| GGETALALLLLDRLN | 0.1 | 0.6 | 1289 |
| GETALALLLLDRLNQ | 0.2 | 0.6 | 1290 |
| ETALALLLLDRLNQL | 0.4 | 0.6 | 1291 |
| TALALLLLDRLNQLE | 0.2 | 0.5 | 1292 |
| ALALLLLDRLNQLES | 0.2 | 0.6 | 1293 |
| LALLLLDRLNQLESK | 0.1 | 0.6 | 1294 |
| ALLLLDRLNQLESKV | 0.1 | 0.4 | 1295 |
| LLLLDRLNQLESKVS | 0.1 | 0.0 | 1296 |
| LLLDRLNQLESKVSG | 0.2 | 0.2 | 1297 |
| LLDRLNQLESKVSGK | 0.6 | 0.1 | 1298 |
| LDRLNQLESKVSGKG | 0.0 | 0.4 | 1299 |
| DRLNQLESKVSGKGQ | 0.3 | 0.4 | 1300 |
| RLNQLESKVSGKGQQ | 0.2 | 0.6 | 1301 |
| LNQLESKVSGKGQQQ | 0.2 | 0.4 | 1302 |
| NQLESKVSGKGQQQQ | 0.2 | 0.5 | 1303 |
| QLESKVSGKGQQQQG | 0.1 | 0.4 | 1304 |
| LESKVSGKGQQQQGQ | 0.1 | 0.6 | 1305 |
| ESKVSGKGQQQQGQT | 0.1 | 0.6 | 1306 |
| SKVSGKGQQQQGQTV | 0.1 | 0.6 | 1307 |
| KVSGKGQQQQGQTVT | 0.2 | 0.4 | 1308 |
| VSGKGQQQQGQTVTK | 0.2 | 0.4 | 1309 |
| SGKGQQQQGQTVTKK | 0.2 | 0.4 | 1310 |
| GKGQQQQGQTVTKKS | 0.2 | 0.4 | 1311 |
| KGQQQQGQTVTKKSA | 0.2 | 0.0 | 1312 |
| GQQQQGQTVTKKSAA | 0.2 | 0.1 | 1313 |
| QQQQGQTVTKKSAAE | 0.3 | 0.0 | 1314 |
| QQQGQTVTKKSAAEA | 0.0 | 0.0 | 1315 |
| QQGQTVTKKSAAEAS | 0.2 | 0.0 | 379 |
| QGQTVTKKSAAEASK | 0.2 | 0.2 | 380 |
| GQTVTKKSAAEASKK | 0.2 | 0.2 | 381 |
| QTVTKKSAAEASKKP | 0.2 | 0.3 | 382 |
| TVTKKSAAEASKKPR | 0.2 | 0.5 | 383 |
| VTKKSAAEASKKPRQ | 0.1 | 0.6 | 384 |
| TKKSAAEASKKPRQK | 0.1 | 0.2 | 385 |
| KKSAAEASKKPRQKR | 0.4 | 0.8 | 386 |
| KSAAEASKKPRQKRT | 0.1 | 0.2 | 387 |
| SAAEASKKPRQKRTA | 0.2 | 0.3 | 388 |
| AAEASKKPRQKRTAT | 0.3 | 0.5 | 389 |
| AEASKKPRQKRTATK | 0.2 | 0.1 | 1316 |
| EASKKPRQKRTATKQ | 0.2 | 0.2 | 1317 |
| ASKKPRQKRTATKQY | 0.2 | 0.4 | 1318 |
| SKKPRQKRTATKQYN | 0.2 | 0.1 | 1319 |
| KKPRQKRTATKQYNV | 0.3 | 0.0 | 1320 |
| KPRQKRTATKQYNVT | 0.3 | 0.0 | 390 |
| 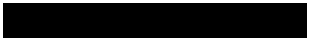 | 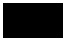 | 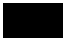 | 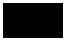 |
| 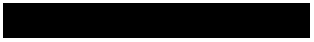 | 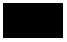 | 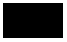 | 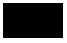 |
| 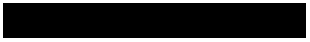 | 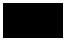 | 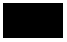 | 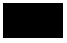 |
| 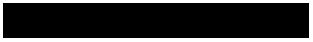 | 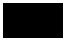 | 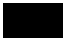 | 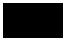 |
| 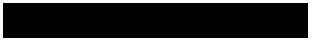 | 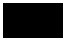 | 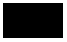 | 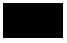 |
| 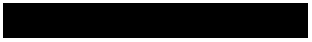 | 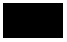 | 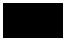 | 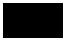 |
| 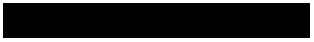 | 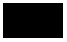 | 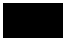 | 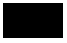 |
| 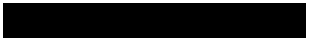 | 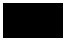 | 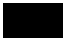 | 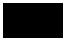 |
| 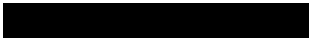 | 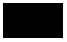 | 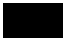 | 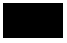 |
| 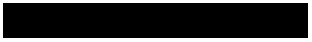 | 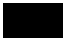 | 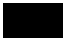 | 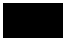 |
| 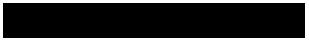 | 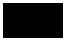 | 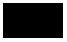 | 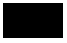 |
| 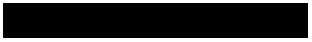 | 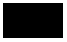 | 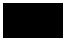 | 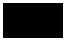 |
| 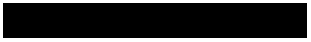 | 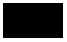 | 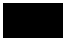 | 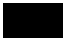 |
| 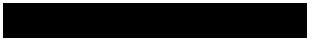 | 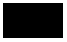 | 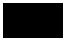 | 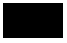 |
| QAFGRRGPEQTQGNF | 0.1 | 0.3 | 1321 |
| AFGRRGPEQTQGNFG | 0.1 | 0.0 | 1322 |
| FGRRGPEQTQGNFGD | 0.2 | 0.0 | 397 |
| GRRGPEQTQGNFGDQ | 0.1 | 0.2 | 398 |
| RRGPEQTQGNFGDQD | 0.1 | 0.1 | 399 |
| RGPEQTQGNFGDQDL | 0.2 | 0.3 | 400 |
| GPEQTQGNFGDQDLI | 0.2 | 0.3 | 401 |
| PEQTQGNFGDQDLIR | 0.1 | 0.5 | 402 |
| EQTQGNFGDQDLIRQ | 0.2 | 0.0 | 403 |
| QTQGNFGDQDLIRQG | 0.1 | 0.6 | 404 |
| TQGNFGDQDLIRQGT | 0.2 | 0.6 | 1323 |
| QGNFGDQDLIRQGTD | 0.2 | 0.4 | 1324 |
| GNFGDQDLIRQGTDY | 0.2 | 0.5 | 1325 |
| NFGDQDLIRQGTDYK | 0.1 | 0.1 | 1326 |
| FGDQDLIRQGTDYKH | 0.1 | 0.5 | 1327 |
| GDQDLIRQGTDYKHW | 0.1 | 0.5 | 1328 |
| DQDLIRQGTDYKHWP | 0.1 | 0.1 | 1329 |
| QDLIRQGTDYKHWPQ | 0.1 | 0.2 | 1330 |
| DLIRQGTDYKHWPQI | 0.0 | 0.5 | 1331 |
| LIRQGTDYKHWPQIA | 0.2 | 0.0 | 1332 |
| TRQGTDYKHWPQIAQ | 0.1 | 0.3 | 1333 |
| RQGTDYKHWPQIAQF | 0.2 | 0.4 | 1334 |
| QGTDYKHWPQIAQFA | 0.2 | 0.3 | 1335 |
| GTDYKHWPQIAQFAP | 0.2 | 0.5 | 1336 |
| TDYKHWPQIAQFAPS | 0.2 | 0.5 | 1337 |
| DYKHWPQIAQFAPSA | 0.1 | 0.5 | 1338 |
| YKHWPQIAQFAPSAS | 0.1 | 0.5 | 1339 |
| KHWPQIAQFAPSASA | 0.2 | 0.0 | 1340 |
| HWPQIAQFAPSASAF | 0.1 | 0.7 | 1341 |
| WPQIAQFAPSASAFF | 0.1 | 0.6 | 1342 |
| PQIAQFAPSASAFFG | 0.1 | 0.5 | 1343 |
| QIAQFAPSASAFFGM | 0.1 | 0.5 | 1344 |
| IAQFAPSASAFFGMS | 0.2 | 0.4 | 1345 |
| AQFAPSASAFFGMSR | 0.1 | 0.7 | 1346 |
| QFAPSASAFFGMSRI | 0.1 | 0.4 | 1347 |
| FAPSASAFFGMSRIG | 0.0 | 0.3 | 1348 |
| APSASAFFGMSRIGM | 0.1 | 0.4 | 1349 |
| PSASAFFGMSRIGME | 0.1 | 0.3 | 1350 |
| SASAFFGMSRIGMEV | 0.1 | 0.6 | 1351 |
| ASAFFGMSRIGMEVT | 0.1 | 0.5 | 1352 |
| SAFFGMSRIGMEVTP | 0.2 | 0.5 | 1353 |
| AFFGMSRIGMEVTPS | 0.1 | 0.5 | 1354 |
| FFGMSRIGMEVTPSG | 0.2 | 0.4 | 1355 |
| FGMSRIGMEVTPSGT | 0.2 | 0.3 | 1356 |
| GMSRIGMEVTPSGTW | 0.1 | 0.4 | 1357 |
| MSRIGMEVTPSGTWL | 0.2 | 0.5 | 1358 |
| SRIGMEVTPSGTWLT | 0.1 | 0.4 | 1359 |
| RIGMEVTPSGTWLTY | 0.0 | 0.5 | 1360 |
| IGMEVTPSGTWLTYH | 0.1 | 0.6 | 1361 |
| GMEVTPSGTWLTYHG | 0.1 | 0.4 | 1362 |
| MEVTPSGTWLTYHGA | 0.1 | 0.4 | 1363 |
| EVTPSGTWLTYHGAI | 0.1 | 0.3 | 1364 |
| VTPSGTWLTYHGAIK | 0.1 | 0.1 | 1365 |
| TPSGTWLTYHGAIKL | 0.1 | 0.4 | 1366 |
| PSGTWLTYHGAIKLD | 0.1 | 0.0 | 1367 |
| SGTWLTYHGAIKLDD | 0.1 | 0.5 | 1368 |
| GTWLTYHGAIKLDDK | 0.1 | 0.2 | 1369 |
| TWLTYHGAIKLDDKD | 0.1 | 0.3 | 1370 |
| WLTYHGAIKLDDKDP | 0.2 | 0.2 | 1371 |
| LTYHGAIKLDDKDPQ | 0.1 | 0.5 | 1372 |
| TYHGAIKLDDKDPQF | 0.1 | 0.2 | 1373 |
| YHGAIKLDDKDPQFK | 0.2 | 0.3 | 1374 |
| HGAIKLDDKDPQFKD | 0.1 | 0.3 | 1375 |
| GAIKLDDKDPQFKDN | 0.1 | 0.3 | 1376 |
| AIKLDDKDPQFKDNV | 0.1 | 0.3 | 1377 |
| IKLDDKDPQFKDNVI | 0.1 | 0.3 | 405 |
| KLDDKDPQFKDNVIL | 0.1 | 0.2 | 406 |
| LDDKDPQFKDNVILL | 0.1 | 0.2 | 407 |
| DDKDPQFKDNVILLN | 0.1 | 0.4 | 408 |
| DKDPQFKDNVILLNK | 0.1 | 0.5 | 409 |
| KDPQFKDNVILLNKH | 0.2 | 0.5 | 410 |
| DPQFKDNVILLNKHI | 0.2 | 0.7 | 411 |
| PQFKDNVILLNKHID | 0.2 | 0.4 | 412 |
| QFKDNVILLNKHIDA | 0.1 | 0.7 | 413 |
| FKDNVILLNKHIDAY | 0.1 | 0.5 | 1378 |
| KDNVILLNKHIDAYK | 0.2 | 0.4 | 1379 |
| DNVILLNKHIDAYKT | 0.1 | 0.6 | 1380 |
| NVILLNKHIDAYKTF | 0.2 | 0.6 | 1381 |
| VILLNKHIDAYKTFP | 0.1 | 0.4 | 1382 |
| ILLNKHIDAYKTFPP | 0.2 | 0.6 | 1383 |
| LLNKHIDAYKTFPPT | 0.2 | 0.4 | 1384 |
| LNKHIDAYKTFPPTE | 0.1 | 0.4 | 1385 |
| NKHIDAYKTFPPTEP | 0.0 | 0.3 | 1386 |
| KHIDAYKTFPPTEPK | 0.1 | 0.2 | 1387 |
| HIDAYKTFPPTEPKK | 0.1 | 0.0 | 1388 |
| IDAYKTFPPTEPKKD | 0.0 | 0.2 | 1389 |
| DAYKTFPPTEPKKDK | 0.1 | 0.1 | 1390 |
| AYKTFPPTEPKKDKK | 0.2 | 0.0 | 1391 |
| YKTFPPTEPKKDKKK | 0.2 | 0.2 | 1392 |
| KTFPPTEPKKDKKKK | 0.1 | 0.1 | 1393 |
| TFPPTEPKKDKKKKT | 0.2 | 0.3 | 1394 |
| FPPTEPKKDKKKKTD | 0.1 | 0.1 | 1395 |
| PPTEPKKDKKKKTDE | 0.2 | 0.2 | 1396 |
| PTEPKKDKKKKTDEA | 0.1 | 0.2 | 1397 |
| TEPKKDKKKKTDEAQ | 0.2 | 0.3 | 1398 |
| EPKKDKKKKTDEAQP | 0.2 | 0.3 | 1399 |
| PKKDKKKKTDEAQPL | 0.1 | 0.0 | 1400 |
| KKDKKKKTDEAQPLP | 0.1 | 0.2 | 1401 |
| KDKKKKTDEAQPLPQ | 0.1 | 0.0 | 1402 |
| DKKKKTDEAQPLPQR | 0.1 | 0.3 | 1403 |
| KKKKTDEAQPLPQRQ | 0.2 | 0.1 | 1404 |
| KKKTDEAQPLPQRQK | 0.2 | 0.1 | 1405 |
| KKTDEAQPLPQRQKK | 0.2 | 0.0 | 1406 |
| KTDEAQPLPQRQKKQ | 0.0 | 0.0 | 1407 |
| TDEAQPLPQRQKKQP | 0.1 | 0.0 | 1408 |
| DEAQPLPQRQKKQPT | 0.1 | 0.0 | 1409 |
| EAQPLPQRQKKQPTV | 0.1 | 0.0 | 1410 |
| AQPLPQRQKKQPTVT | 0.2 | 0.2 | 1411 |
| QPLPQRQKKQPTVTL | 0.1 | 0.7 | 414 |
| PLPQRQKKQPTVTLL | 0.1 | 0.7 | 415 |
| LPQRQKKQPTVTLLP | 0.2 | 0.7 | 416 |
| PQRQKKQPTVTLLPA | 0.2 | 0.7 | 417 |
| QRQKKQPTVTLLPAA | 0.2 | 0.7 | 418 |
| RQKKQPTVTLLPAAD | 0.1 | 0.4 | 419 |
| QKKQPTVTLLPAADM | 0.2 | 0.7 | 420 |
| KKQPTVTLLPAADMD | 0.1 | 0.2 | 1412 |
| KQPTVTLLPAADMDD | 0.1 | 0.1 | 1413 |
| QPTVTLLPAADMDDF | 0.1 | 0.0 | 1414 |
| PTVTLLPAADMDDFS | 0.1 | 0.0 | 1415 |
| TVTLLPAADMDDFSR | 0.1 | 0.3 | 1416 |
| VTLLPAADMDDFSRQ | 0.0 | 0.0 | 1417 |
| TLLPAADMDDFSRQL | 0.1 | 0.0 | 1418 |
| LLPAADMDDFSRQLQ | 0.1 | 0.2 | 1419 |
| LPAADMDDFSRQLQN | 0.2 | 0.2 | 1420 |
| PAADMDDFSRQLQNS | 0.2 | 0.3 | 1421 |
| AADMDDFSRQLQNSM | 0.2 | 0.3 | 1422 |
| ADMDDFSRQLQNSMS | 0.2 | 0.4 | 1423 |
| DMDDFSRQLQNSMSG | 0.2 | 0.4 | 1424 |
| MDDFSRQLQNSMSGA | 0.2 | 0.4 | 1425 |
| DDFSRQLQNSMSGAS | 0.5 | 0.3 | 1426 |
| DFSRQLQNSMSGASA | 0.5 | 0.6 | 1427 |
| FSRQLQNSMSGASAD | 0.4 | 0.1 | 1428 |
| SRQLQNSMSGASADS | 0.5 | 0.6 | 1429 |
| RQLQNSMSGASADST | 0.5 | 0.3 | 1430 |
| QLQNSMSGASADSTQ | 0.7 | 0.5 | 1431 |
| LQNSMSGASADSTQA | 0.9 | 0.4 | 1432 |

TABLE 33
Binding of single-chain (scFv) phage
antibodies to a SARS-CoV preparation
(Frankfurt 1 strain) and to FBS as measured by ELISA.

| | SARS-CoV preparation | FBS |
|---|---|---|
| Name phage antibody | (OD492nm) | (OD492nm) |
| SC03-001 | 0.979 | 0.142 |
| SC03-002 | 0.841 | 0.091 |
| SC03-003 | 0.192 | 0.092 |
| SC03-005 | 0.869 | 0.098 |
| SC03-006 | 1.056 | 0.086 |
| SC03-007 | 0.876 | 0.096 |
| SC03-008 | 0.358 | 0.114 |
| SC03-009 | 0.760 | 0.087 |
| SC03-010 | 0.327 | 0.082 |
| SC03-012 | 0.495 | 0.100 |
| SC03-013 | 0.979 | 0.101 |
| SC03-014 | 0.917 | 0.089 |
| SC03-015 | 0.796 | 0.077 |
| Anti-thyroglobulin | 0.108 | 0.090 |
| (SC02-006) | | |
| No phage antibody | 0.072 | 0.083 |

TABLE 34
Binding of alternatively selected single-chain (scFv)
phage antibodies to a SARS-CoV preparation (Frankfurt
1 strain) and to FBS as measured by ELISA.

| | SARS-CoV preparation | FBS |
|---|---|---|
| Name phage antibody | (OD492nm) | (OD492nm) |
| SC03-016 | 0.313 | 0.205 |
| SC03-017 | 0.106 | 0.059 |
| SC03-018 | 1.523 | 0.072 |
| Anti-CD46 (SC02-300) | 0.171 | 0.070 |
| No phage antibody | 0.081 | 0.045 |

TABLE 35
Binding of antibody 03-018 to linear and looped/cyclic
peptides of the N protein of SARS-CoV Urbani.

| | Antibody | | |
|---|---|---|---|
| Peptides of N | 03-018 | Antibody 03-018 | SEQ |
| protein | linear peptides | looped peptides | ID NO |
| MSDNGPQSNQRSAPR | 0.1 | 0.3 | 1123 |
| SDNGPQSNQRSAPRI | 0.0 | 0.2 | 1124 |
| DNGPQSNQRSAPRIT | 0.2 | 0.3 | 1125 |
| 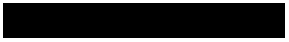 | 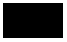 | 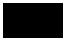 | 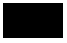 |
| 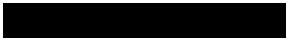 | 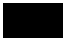 | 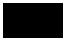 | 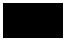 |
| 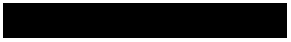 | 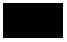 | 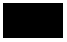 | 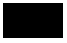 |
| 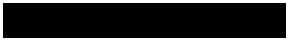 | 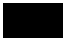 | 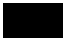 | 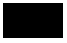 |
| 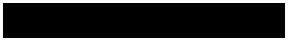 | 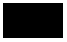 | 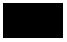 | 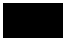 |
| 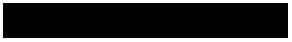 | 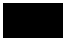 | 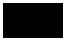 | 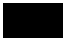 |
| 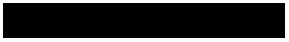 | 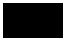 | 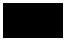 | 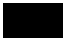 |
| 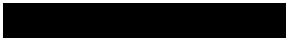 | 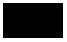 | 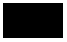 | 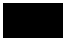 |
| 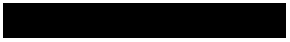 | 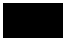 | 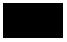 | 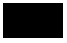 |
| 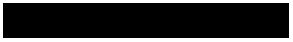 | 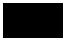 | 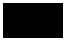 | 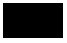 |
| 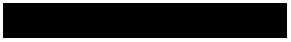 | 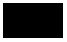 | 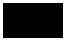 | 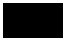 |
| 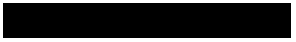 | 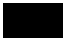 | 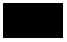 | 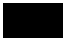 |
| 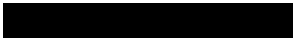 | 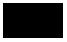 | 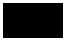 | 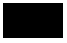 |
| TFGGPTDSTDNNQNG | 0.1 | 0.2 | 1126 |
| FGGPTDSTDNNQNGG | 0.1 | 0.2 | 1127 |
| GGPTDSTDNNQNGGR | 0.1 | 0.2 | 1128 |
| GPTDSTDNNQNGGRN | 0.2 | 0.2 | 1129 |
| PTDSTDNNQNGGRNG | 0.1 | 0.2 | 1130 |
| TDSTDNNQNGGRNGA | 0.2 | 0.2 | 1131 |
| DSTDNNQNGGRNGAR | 0.2 | 0.3 | 1132 |
| STDNNQNGGRNGARP | 0.2 | 0.2 | 1133 |
| TDNNQNGGRNGARPK | 0.2 | 0.2 | 1134 |
| DNNQNGGRNGARPKQ | 0.2 | 0.3 | 1135 |
| NNQNGGRNGARPKQR | 0.2 | 0.2 | 1136 |
| NQNGGRNGARPKQRR | 0.2 | 0.2 | 1137 |
| QNGGRNGARPKQRRP | 0.2 | 0.3 | 1138 |
| NGGRNGARPKQRRPQ | 0.2 | 0.3 | 1139 |
| GGRNGARPKQRRPQG | 0.2 | 0.2 | 1140 |
| GRNGARPKQRRPQGL | 0.1 | 0.2 | 1141 |
| RNGARPKQRRPQGLP | 0.1 | 0.3 | 1142 |
| NGARPKQRRPQGLPN | 0.1 | 0.3 | 1143 |
| GARPKQRRPQGLPNN | 0.1 | 0.2 | 1144 |
| ARPKQRRPQGLPNNT | 0.1 | 0.2 | 1145 |
| RPKQRRPQGLPNNTA | 0.1 | 0.2 | 1146 |
| PKQRRPQGLPNNTAS | 0.2 | 0.3 | 1147 |
| KQRRPQGLPNNTASW | 0.1 | 0.2 | 1148 |
| QRRPQGLPNNTASWF | 0.1 | 0.2 | 1149 |
| RRPQGLPNNTASWFT | 0.1 | 0.2 | 1150 |
| RPQGLPNNTASWFTA | 0.1 | 0.2 | 1151 |
| PQGLPNNTASWFTAL | 0.1 | 0.3 | 1152 |
| QGLPNNTASWFTALT | 0.1 | 0.3 | 1153 |
| GLPNNTASWFTALTQ | 0.1 | 0.3 | 1154 |
| LPNNTASWFTALTQH | 0.1 | 0.3 | 1155 |
| PNNTASWFTALTQHG | 0.1 | 0.3 | 1156 |
| NNTASWFTALTQHGK | 0.1 | 0.2 | 1157 |
| NTASWFTALTQHGKE | 0.1 | 0.2 | 1158 |
| TASWFTALTQHGKEE | 0.1 | 0.2 | 1159 |
| ASWFTALTQHGKEEL | 0.1 | 0.2 | 1160 |
| SWFTALTQHGKEELR | 0.1 | 0.2 | 1161 |
| WFTALTQHGKEELRF | 0.1 | 0.2 | 1162 |
| FTALTQHGKEELRFP | 0.1 | 0.2 | 1163 |
| TALTQHGKEELRFPR | 0.1 | 0.3 | 1164 |
| ALTQHGKEELRFPRG | 0.2 | 0.2 | 1165 |
| LTQHGKEELRFPRGQ | 0.1 | 0.2 | 1166 |
| TQHGKEELRFPRGQG | 0.1 | 0.2 | 1167 |
| QHGKEELRFPRGQGV | 0.1 | 0.2 | 1168 |
| HGKEELRFPRGQGVP | 0.1 | 0.2 | 1169 |
| GKEELRFPRGQGVPI | 0.1 | 0.3 | 1170 |
| KEELRFPRGQGVPIN | 0.1 | 0.3 | 1171 |
| EELRFPRGQGVPINT | 0.1 | 0.3 | 1172 |
| ELRFPRGQGVPINTN | 0.1 | 0.2 | 1173 |
| LRFPRGQGVPINTNS | 0.1 | 0.2 | 1174 |
| RFPRGQGVPINTNSG | 0.1 | 0.2 | 1175 |
| FPRGQGVPINTNSGP | 0.1 | 0.2 | 1176 |
| PRGQGVPINTNSGPD | 0.1 | 0.2 | 1177 |
| RGQGVPINTNSGPDD | 0.1 | 0.2 | 1178 |
| GQGVPINTNSGPDDQ | 0.1 | 0.2 | 1179 |
| QGVPINTNSGPDDQI | 0.1 | 0.1 | 1180 |
| GVPINTNSGPDDQIG | 0.1 | 0.2 | 1181 |
| VPINTNSGPDDQIGY | 0.1 | 0.2 | 1182 |
| PINTNSGPDDQIGYY | 0.1 | 0.2 | 1183 |
| INTNSGPDDQIGYYR | 0.1 | 0.2 | 1184 |
| NTNSGPDDQIGYYRR | 0.1 | 0.3 | 1185 |
| TNSGPDDQIGYYRRA | 0.1 | 0.2 | 1186 |
| NSGPDDQIGYYRRAT | 0.1 | 0.2 | 1187 |
| SGPDDQIGYYRRATR | 0.1 | 0.3 | 545 |
| GPDDQTGYYRRATRR | 0.1 | 0.3 | 546 |
| PDDQIGYYRRATRRV | 0.1 | 0.3 | 547 |
| DDQIGYYRRATRRVR | 0.1 | 0.3 | 548 |
| DQIGYYRRATRRVRG | 0.1 | 0.3 | 549 |
| QIGYYRRATRRVRGG | 0.1 | 0.2 | 550 |
| IGYYRRATRRVRGGD | 0.1 | 0.2 | 551 |
| GYYRRATRRVRGGDG | 0.1 | 0.2 | 552 |
| YYRRATRRVRGGDGK | 0.1 | 0.2 | 1188 |
| YRRATRRVRGGDGKM | 0.1 | 0.2 | 1189 |
| RRATRRVRGGDGKMK | 0.1 | 0.2 | 1190 |
| RATRRVRGGDGKMKE | 0.1 | 0.2 | 1191 |
| ATRRVRGGDGKMKEL | 0.1 | 0.2 | 1192 |
| TRRVRGGDGKMKELS | 0.1 | 0.2 | 1193 |
| RRVRGGDGKMKELSP | 0.1 | 0.2 | 1194 |
| RVRGGDGKMKELSPR | 0.1 | 0.2 | 1195 |
| VRGGDGKMKELSPRW | 0.1 | 0.2 | 1196 |
| RGGDGKMKELSPRWY | 0.1 | 0.2 | 1197 |
| GGDGKMKELSPRWYF | 0.1 | 0.2 | 1198 |
| GDGKMKELSPRWYFY | 0.1 | 0.2 | 1199 |
| DGKMKELSPRWYFYY | 0.1 | 0.2 | 1200 |
| GKMKELSPRWYFYYL | 0.1 | 0.3 | 1201 |
| KMKELSPRWYFYYLG | 0.1 | 0.2 | 1202 |
| MKELSPRWYFYYLGT | 0.1 | 0.2 | 1203 |
| KELSPRWYFYYLGTG | 0.1 | 0.3 | 1204 |
| ELSPRWYFYYLGTGP | 0.1 | 0.2 | 1205 |
| LSPRWYFYYLGTGPE | 0.1 | 0.2 | 1206 |
| SPRWYFYYLGTGPEA | 0.1 | 0.2 | 1207 |
| PRWYFYYLGTGPEAS | 0.1 | 0.2 | 1208 |
| RWYFYYLGTGPEASL | 0.1 | 0.2 | 1209 |
| WYFYYLGTGPEASLP | 0.1 | 0.2 | 1210 |
| YFYYLGTGPEASLPY | 0.1 | 0.2 | 1211 |
| FYYLGTGPEASLPYG | 0.1 | 0.2 | 1212 |
| YYLGTGPEASLPYGA | 0.1 | 0.2 | 1213 |
| YLGTGPEASLPYGAN | 0.1 | 0.2 | 1214 |
| LGTGPEASLPYGANK | 0.1 | 0.2 | 1215 |
| GTGPEASLPYGANKE | 0.1 | 0.2 | 1216 |
| TGPEASLPYGANKEG | 0.1 | 0.2 | 1217 |
| GPEASLPYGANKEGI | 0.1 | 0.2 | 1218 |
| PEASLPYGANKEGIV | 0.1 | 0.2 | 1219 |
| EASLPYGANKEGIVW | 0.1 | 0.2 | 1220 |
| ASLPYGANKEGIVWV | 0.1 | 0.3 | 1221 |
| SLPYGANKEGIVWVA | 0.1 | 0.2 | 1222 |
| LPYGANKEGIVWVAT | 0.1 | 0.2 | 1223 |
| PYGANKEGIVWVATE | 0.1 | 0.2 | 1224 |
| YGANKEGIVWVATEG | 0.1 | 0.2 | 1225 |
| GANKEGIVWVATEGA | 0.1 | 0.2 | 1226 |
| ANKEGIVWVATEGAL | 0.1 | 0.2 | 1227 |
| NKEGIVWVATEGALN | 0.1 | 0.2 | 1228 |
| KEGIVWVATEGALNT | 0.1 | 0.2 | 1229 |
| EGIVWVATEGALNTP | 0.1 | 0.2 | 1230 |
| GIVWVATEGALNTPK | 0.1 | 0.2 | 1231 |
| IVWVATEGALNTPKD | 0.1 | 0.2 | 1232 |
| VWVATEGALNTPKDH | 0.1 | 0.3 | 1233 |
| WVATEGALNTPKDHI | 0.1 | 0.2 | 1234 |
| VATEGALNTPKDHIG | 0.2 | 0.2 | 1235 |
| ATEGALNTPKDHIGT | 0.1 | 0.2 | 1236 |
| TEGALNTPKDHTGTR | 0.2 | 0.3 | 1237 |
| EGALNTPKDHIGTRN | 0.1 | 0.3 | 1238 |
| GALNTPKDHIGTRNP | 0.1 | 0.2 | 1239 |
| ALNTPKDHIGTRNPN | 0.1 | 0.2 | 1240 |
| LNTPKDHIGTRNPNN | 0.1 | 0.2 | 1241 |
| NTPKDHIGTRNPNNN | 0.1 | 0.2 | 1242 |
| TPKDHIGTRNPNNNA | 0.1 | 0.2 | 1243 |
| PKDHIGTRNPNNNAA | 0.1 | 0.2 | 1244 |
| KDHIGTRNPNNNAAT | 0.1 | 0.2 | 1245 |
| DHIGTRNPNNNAATV | 0.1 | 0.3 | 1246 |
| HIGTRNPNNNAATVL | 0.1 | 0.3 | 1247 |
| IGTRNPNNNAATVLQ | 0.1 | 0.3 | 1248 |
| GTRNPNNNAATVLQL | 0.1 | 0.3 | 1249 |
| TRNPNNNAATVLQLP | 0.1 | 0.2 | 1250 |
| RNPNNNAATVLQLPQ | 0.1 | 0.2 | 1251 |
| NPNNNAATVLQLPQG | 0.1 | 0.3 | 1252 |
| PNNNAATVLQLPQGT | 0.1 | 0.3 | 1253 |
| NNNAATVLQLPQGTT | 0.1 | 0.3 | 1254 |
| NNAATVLQLPQGTTL | 0.1 | 0.3 | 358 |
| NAATVLQLPQGTTLP | 0.1 | 0.2 | 359 |
| AATVLQLPQGTTLPK | 0.1 | 0.2 | 360 |
| ATVLQLPQGTTLPKG | 0.1 | 0.2 | 361 |
| TVLQLPQGTTLPKGF | 0.1 | 0.3 | 362 |
| VLQLPQGTTLPKGFY | 0.1 | 0.3 | 363 |
| LQLPQGTTLPKGFYA | 0.1 | 0.2 | 364 |
| QLPQGTTLPKGFYAE | 0.1 | 0.2 | 365 |
| LPQGTTLPKGFYAEG | 0.1 | 0.3 | 366 |
| PQGTTLPKGFYAEGS | 0.1 | 0.2 | 367 |
| QGTTLPKGFYAEGSR | 0.1 | 0.2 | 368 |
| GTTLPKGFYAEGSRG | 0.1 | 0.2 | 369 |
| TTLPKGFYAEGSRGG | 0.1 | 0.2 | 370 |
| TLPKGFYAEGSRGGS | 0.1 | 0.2 | 371 |
| LPKGFYAEGSRGGSQ | 0.1 | 0.2 | 1255 |
| PKGFYAEGSRGGSQA | 0.1 | 0.2 | 1256 |
| KGFYAEGSRGGSQAS | 0.1 | 0.2 | 1257 |
| GFYAEGSRGGSQASS | 0.1 | 0.2 | 1258 |
| FYAEGSRGGSQASSR | 0.1 | 0.1 | 1259 |
| YAEGSRGGSQASSRS | 0.1 | 0.2 | 1260 |
| AEGSRGGSQASSRSS | 0.1 | 0.2 | 1261 |
| EGSRGGSQASSRSSS | 0.1 | 0.2 | 1262 |
| GSRGGSQASSRSSSR | 0.1 | 0.2 | 1263 |
| SRGGSQASSRSSSRS | 0.1 | 0.2 | 1264 |
| RGGSQASSRSSSRSR | 0.1 | 0.1 | 1265 |
| GGSQASSRSSSRSRG | 0.1 | 0.2 | 1266 |
| GSQASSRSSSRSRGN | 0.1 | 0.2 | 1267 |
| SQASSRSSSRSRGNS | 0.1 | 0.2 | 1268 |
| QASSRSSSRSRGNSR | 0.1 | 0.2 | 1269 |
| ASSRSSSRSRGNSRN | 0.1 | 0.2 | 1270 |
| SSRSSSRSRGNSRNS | 0.1 | 0.2 | 1271 |
| SRSSSRSRGNSRNST | 0.1 | 0.2 | 1272 |
| RSSSRSRGNSRNSTP | 0.1 | 0.2 | 1273 |
| SSSRSRGNSRNSTPG | 0.1 | 0.2 | 1274 |
| SSRSRGNSRNSTPGS | 0.1 | 0.2 | 1275 |
| SRSRGNSRNSTPGSS | 0.1 | 0.2 | 1276 |
| RSRGNSRNSTPGSSR | 0.1 | 0.2 | 1277 |
| SRGNSRNSTPGSSRG | 0.1 | 0.2 | 1278 |
| RGNSRNSTPGSSRGN | 0.1 | 0.2 | 1279 |
| GNSRNSTPGSSRGNS | 0.1 | 0.2 | 1280 |
| NSRNSTPGSSRGNSP | 0.1 | 0.2 | 1281 |
| SRNSTPGSSRGNSPA | 0.1 | 0.2 | 1282 |
| RNSTPGSSRGNSPAR | 0.1 | 0.2 | 553 |
| NSTPGSSRGNSPARM | 0.2 | 0.3 | 554 |
| STPGSSRGNSPARMA | 0.1 | 0.2 | 555 |
| TPGSSRGNSPARMAS | 0.1 | 0.3 | 556 |
| PGSSRGNSPARMASG | 0.1 | 0.3 | 557 |
| GSSRGNSPARMASGG | 0.1 | 0.2 | 558 |
| SSRGNSPARMASGGG | 0.1 | 0.2 | 1283 |
| SRGNSPARMASGGGE | 0.1 | 0.2 | 1284 |
| RGNSPARMASGGGET | 0.1 | 0.2 | 1285 |
| GNSPARMASGGGETA | 0.2 | 0.2 | 1286 |
| NSPARMASGGGETAL | 0.1 | 0.2 | 372 |
| SPARMASGGGETALA | 0.1 | 0.1 | 373 |
| PARMASGGGETALAL | 0.1 | 0.3 | 374 |
| ARMASGGGETALALL | 0.1 | 0.3 | 375 |
| RMASGGGETALALLL | 0.1 | 0.3 | 376 |
| MASGGGETALALLLL | 0.1 | 0.3 | 377 |
| ASGGGETALALLLLD | 0.1 | 0.2 | 378 |
| SGGGETALALLLLDR | 0.1 | 0.2 | 1287 |
| GGGETALALLLLDRL | 0.1 | 0.2 | 1288 |
| GGETALALLLLDRLN | 0.1 | 0.2 | 1289 |
| GETALALLLLDRLNQ | 0.1 | 0.3 | 1290 |
| ETALALLLLDRLNQL | 0.1 | 0.3 | 1291 |
| TALALLLLDRLNQLE | 0.1 | 0.2 | 1292 |
| ALALLLLDRLNQLES | 0.1 | 0.3 | 1293 |
| LALLLLDRLNQLESK | 0.1 | 0.2 | 1294 |
| ALLLLDRLNQLESKV | 0.1 | 0.3 | 1295 |
| LLLLDRLNQLESKVS | 0.2 | 0.2 | 1296 |
| LLLDRLNQLESKVSG | 0.1 | 0.2 | 1297 |
| LLDRLNQLESKVSGK | 0.1 | 0.2 | 1298 |
| LDRLNQLESKVSGKG | 0.1 | 0.2 | 1299 |
| DRLNQLESKVSGKGQ | 0.1 | 0.3 | 1300 |
| RLNQLESKVSGKGQQ | 0.1 | 0.2 | 1301 |
| LNQLESKVSGKGQQQ | 0.1 | 0.3 | 1302 |
| NQLESKVSGKGQQQQ | 0.1 | 0.3 | 1303 |
| QLESKVSGKGQQQQG | 0.1 | 0.3 | 1304 |
| LESKVSGKGQQQQGQ | 0.1 | 0.3 | 1305 |
| ESKVSGKGQQQQGQT | 0.1 | 0.2 | 1306 |
| SKVSGKGQQQQGQTV | 0.1 | 0.2 | 1307 |
| KVSGKGQQQQGQTVT | 0.1 | 0.2 | 1308 |
| VSGKGQQQQGQTVTK | 0.1 | 0.3 | 1309 |
| SGKGQQQQGQTVTKK | 0.1 | 0.2 | 1310 |
| GKGQQQQGQTVTKKS | 0.1 | 0.2 | 1311 |
| KGQQQQGQTVTKKSA | 0.1 | 0.2 | 1312 |
| GQQQQGQTVTKKSAA | 0.1 | 0.2 | 1313 |
| QQQQGQTVTKKSAAE | 0.1 | 0.2 | 1314 |
| QQQGQTVTKKSAAEA | 0.1 | 0.2 | 1315 |
| QQGQTVTKKSAAEAS | 0.1 | 0.2 | 379 |
| QGQTVTKKSAAEASK | 0.1 | 0.2 | 380 |
| GQTVTKKSAAEASKK | 0.1 | 0.2 | 381 |
| QTVTKKSAAEASKKP | 0.1 | 0.2 | 382 |
| TVTKKSAAEASKKPR | 0.1 | 0.2 | 383 |
| VTKKSAAEASKKPRQ | 0.1 | 0.2 | 384 |
| TKKSAAEASKKPRQK | 0.1 | 0.2 | 385 |
| KKSAAEASKKPRQKR | 0.1 | 0.2 | 386 |
| KSAAEASKKPRQKRT | 0.1 | 0.1 | 387 |
| SAAEASKKPRQKRTA | 0.1 | 0.2 | 388 |
| AAEASKKPRQKRTAT | 0.1 | 0.2 | 389 |
| AEASKKPRQKRTATK | 0.1 | 0.2 | 1316 |
| EASKKPRQKRTATKQ | 0.1 | 0.3 | 1317 |
| ASKKPRQKRTATKQY | 0.1 | 0.2 | 1318 |
| SKKPRQKRTATKQYN | 0.1 | 0.2 | 1319 |
| KKPRQKRTATKQYNV | 0.1 | 0.2 | 1320 |
| KPRQKRTATKQYNVT | 0.1 | 0.2 | 390 |
| PRQKRTATKQYNVTQ | 0.1 | 0.2 | 391 |
| RQKRTATKQYNVTQA | 0.1 | 0.2 | 392 |
| QKRTATKQYNVTQAF | 0.1 | 0.2 | 393 |
| KRTATKQYNVTQAFG | 0.1 | 0.2 | 394 |
| RTATKQYNVTQAFGR | 0.1 | 0.2 | 395 |
| TATKQYNVTQAFGRR | 0.1 | 0.3 | 396 |
| ATKQYNVTQAFGRRG | 0.1 | 0.3 | 565 |
| TKQYNVTQAFGRRGP | 0.1 | 0.3 | 566 |
| KQYNVTQAFGRRGPE | 0.1 | 0.1 | 567 |
| QYNVTQAFGRRGPEQ | 0.1 | 0.3 | 568 |
| YNVTQAFGRRGPEQT | 0.1 | 0.2 | 569 |
| NVTQAFGRRGPEQTQ | 0.1 | 0.2 | 570 |
| VTQAFGRRGPEQTQG | 0.1 | 0.2 | 571 |
| TQAFGRRGPEQTQGN | 0.1 | 0.2 | 572 |
| QAFGRRGPEQTQGNF | 0.1 | 0.2 | 1321 |
| AFGRRGPEQTQGNFG | 0.1 | 0.2 | 1322 |
| FGRRGPEQTQGNFGD | 0.1 | 0.1 | 397 |
| GRRGPEQTQGNFGDQ | 0.1 | 0.2 | 398 |
| RRGPEQTQGNFGDQD | 0.1 | 0.2 | 399 |
| RGPEQTQGNFGDQDL | 0.1 | 0.2 | 400 |
| GPEQTQGNFGDQDLI | 0.1 | 0.2 | 401 |
| PEQTQGNFGDQDLIR | 0.1 | 0.2 | 402 |
| EQTQGNFGDQDLIRQ | 0.1 | 0.0 | 403 |
| QTQGNFGDQDLIRQG | 0.1 | 0.2 | 404 |
| TQGNFGDQDLIRQGT | 0.1 | 0.2 | 1323 |
| QGNFGDQDLIRQGTD | 0.1 | 0.2 | 1324 |
| GNFGDQDLIRQGTDY | 0.1 | 0.2 | 1325 |
| NFGDQDLIRQGTDYK | 0.1 | 0.2 | 1326 |
| FGDQDLIRQGTDYKH | 0.1 | 0.2 | 1327 |
| GDQDLIRQGTDYKHW | 0.1 | 0.2 | 1328 |
| DQDLIRQGTDYKHWP | 0.1 | 0.2 | 1329 |
| QDLIRQGTDYKHWPQ | 0.1 | 0.2 | 1330 |
| DLIRQGTDYKHWPQI | 0.1 | 0.2 | 1331 |
| LIRQGTDYKHWPQIA | 0.1 | 0.1 | 1332 |
| IRQGTDYKHWPQIAQ | 0.1 | 0.2 | 1333 |
| RQGTDYKHWPQIAQF | 0.1 | 0.2 | 1334 |
| QGTDYKHWPQIAQFA | 0.1 | 0.2 | 1335 |
| GTDYKHWPQIAQFAP | 0.1 | 0.2 | 1336 |
| TDYKHWPQIAQFAPS | 0.1 | 0.2 | 1337 |
| DYKHWPQIAQFAPSA | 0.1 | 0.2 | 1338 |
| YKHWPQIAQFAPSAS | 0.1 | 0.2 | 1339 |
| KHWPQIAQFAPSASA | 0.1 | 0.2 | 1340 |
| HWPQIAQFAPSASAF | 0.1 | 0.2 | 1341 |
| WPQIAQFAPSASAFF | 0.1 | 0.3 | 1342 |
| PQIAQFAPSASAFFG | 0.1 | 0.2 | 1343 |
| QIAQFAPSASAFFGM | 0.1 | 0.3 | 1344 |
| IAQFAPSASAFFGMS | 0.1 | 0.3 | 1345 |
| AQFAPSASAFFGMSR | 0.1 | 0.3 | 1346 |
| QFAPSASAFFGMSRI | 0.1 | 0.3 | 1347 |
| FAPSASAFFGMSRIG | 0.1 | 0.2 | 1348 |
| APSASAFFGMSRIGM | 0.1 | 0.2 | 1349 |
| PSASAFFGMSRIGME | 0.1 | 0.2 | 1350 |
| SASAFFGMSRIGMEV | 0.1 | 0.2 | 1351 |
| ASAFFGMSRIGMEVT | 0.1 | 0.2 | 1352 |
| SAFFGMSRIGMEVTP | 0.1 | 0.2 | 1353 |
| AFFGMSRIGMEVTPS | 0.1 | 0.2 | 1354 |
| FFGMSRIGMEVTPSG | 0.1 | 0.2 | 1355 |
| FGMSRIGMEVTPSGT | 0.1 | 0.2 | 1356 |
| GMSRIGMEVTPSGTW | 0.1 | 0.2 | 1357 |
| MSRIGMEVTPSGTWL | 0.1 | 0.2 | 1358 |
| SRIGMEVTPSGTWLT | 0.1 | 0.2 | 1359 |
| RIGMEVTPSGTWLTY | 0.1 | 0.2 | 1360 |
| IGMEVTPSGTWLTYH | 0.1 | 0.2 | 1361 |
| GMEVTPSGTWLTYHG | 0.1 | 0.2 | 1362 |
| MEVTPSGTWLTYHGA | 0.1 | 0.2 | 1363 |
| EVTPSGTWLTYHGAI | 0.1 | 0.2 | 1364 |
| VTPSGTWLTYHGAIK | 0.1 | 0.2 | 1365 |
| TPSGTWLTYHGAIKL | 0.1 | 0.2 | 1366 |
| PSGTWLTYHGAIKLD | 0.1 | 0.2 | 1367 |
| SGTWLTYHGAIKLDD | 0.1 | 0.2 | 1368 |
| GTWLTYHGAIKLDDK | 0.1 | 0.2 | 1369 |
| TWLTYHGAIKLDDKD | 0.1 | 0.2 | 1370 |
| WLTYHGAIKLDDKDP | 0.1 | 0.2 | 1371 |
| LTYHGAIKLDDKDPQ | 0.1 | 0.2 | 1372 |
| TYHGAIKLDDKDPQF | 0.1 | 0.1 | 1373 |
| YHGAIKLDDKDPQFK | 0.1 | 0.2 | 1374 |
| HGAIKLDDKDPQFKD | 0.1 | 0.2 | 1375 |
| GAIKLDDKDPQFKDN | 0.1 | 0.2 | 1376 |
| AIKLDDKDPQFKDNV | 0.1 | 0.2 | 1377 |
| IKLDDKDPQFKDNVI | 0.1 | 0.2 | 405 |
| KLDDKDPQFKDNVIL | 0.1 | 0.2 | 406 |
| LDDKDPQFKDNVILL | 0.1 | 0.3 | 407 |
| DDKDPQFKDNVILLN | 0.1 | 0.3 | 408 |
| DKDPQFKDNVILLNK | 0.1 | 0.4 | 409 |
| KDPQFKDNVILLNKH | 0.1 | 0.2 | 410 |
| DPQFKDNVILLNKHI | 0.1 | 0.3 | 411 |
| PQFKDNVILLNKHID | 0.1 | 0.2 | 412 |
| QFKDNVILLNKHIDA | 0.1 | 0.3 | 413 |
| FKDNVILLNKHIDAY | 0.1 | 0.2 | 1378 |
| KDNVILLNKHIDAYK | 0.1 | 0.2 | 1379 |
| DNVILLNKHIDAYKT | 0.1 | 0.2 | 1380 |
| NVILLNKHIDAYKTF | 0.1 | 0.2 | 1381 |
| VILLNKHIDAYKTFP | 0.1 | 0.2 | 1382 |
| ILLNKHIDAYKTFPP | 0.1 | 0.2 | 1383 |
| LLNKHIDAYKTFPPT | 0.1 | 0.2 | 1384 |
| LNKHIDAYKTFPPTE | 0.1 | 0.2 | 1385 |
| NKHIDAYKTFPPTEP | 0.1 | 0.2 | 1386 |
| KHIDAYKTFPPTEPK | 0.1 | 0.2 | 1387 |
| HIDAYKTFPPTEPKK | 0.1 | 0.2 | 1388 |
| IDAYKTFPPTEPKKD | 0.1 | 0.2 | 1389 |
| DAYKTFPPTEPKKDK | 0.1 | 0.2 | 1390 |
| AYKTFPPTEPKKDKK | 0.1 | 0.1 | 1391 |
| YKTFPPTEPKKDKKK | 0.1 | 0.2 | 1392 |
| KTFPPTEPKKDKKKK | 0.1 | 0.2 | 1393 |
| TFPPTEPKKDKKKKT | 0.1 | 0.2 | 1394 |
| FPPTEPKKDKKKKTD | 0.1 | 0.2 | 1395 |
| PPTEPKKDKKKKTDE | 0.1 | 0.2 | 1396 |
| PTEPKKDKKKKTDEA | 0.1 | 0.2 | 1397 |
| TEPKKDKKKKTDEAQ | 0.1 | 0.2 | 1398 |
| EPKKDKKKKTDEAQP | 0.1 | 0.2 | 1399 |
| PKKDKKKKTDEAQPL | 0.1 | 0.2 | 1400 |
| KKDKKKKTDEAQPLP | 0.1 | 0.2 | 1401 |
| KDKKKKTDEAQPLPQ | 0.1 | 0.2 | 1402 |
| DKKKKTDEAQPLPQR | 0.1 | 0.2 | 1403 |
| KKKKTDEAQPLPQRQ | 0.1 | 0.2 | 1404 |
| KKKTDEAQPLPQRQK | 0.1 | 0.2 | 1405 |
| KKTDEAQPLPQRQKK | 0.1 | 0.2 | 1406 |
| KTDEAQPLPQRQKKQ | 0.1 | 0.2 | 1407 |
| TDEAQPLPQRQKKQP | 0.1 | 0.1 | 1408 |
| DEAQPLPQRQKKQPT | 0.1 | 0.2 | 1409 |
| EAQPLPQRQKKQPTV | 0.1 | 0.2 | 1410 |
| AQPLPQRQKKQPTVT | 0.1 | 0.1 | 1411 |
| QPLPQRQKKQPTVTL | 0.1 | 0.3 | 414 |
| PLPQRQKKQPTVTLL | 0.1 | 0.3 | 415 |
| LPQRQKKQPTVTLLP | 0.1 | 0.3 | 416 |
| PQRQKKQPTVTLLPA | 0.1 | 0.3 | 417 |
| QRQKKQPTVTLLPAA | 0.1 | 0.3 | 418 |
| RQKKQPTVTLLPAAD | 0.1 | 0.2 | 419 |
| QKKQPTVTLLPAADM | 0.1 | 0.3 | 420 |
| KKQPTVTLLPAADMD | 0.1 | 0.2 | 1412 |
| KQPTVTLLPAADMDD | 0.1 | 0.2 | 1413 |
| QPTVTLLPAADMDDF | 0.1 | 0.2 | 1414 |
| PTVTLLPAADMDDFS | 0.1 | 0.2 | 1415 |
| TVTLLPAADMDDFSR | 0.1 | 0.2 | 1416 |
| VTLLPAADMDDFSRQ | 0.1 | 0.2 | 1417 |
| TLLPAADMDDFSRQL | 0.1 | 0.1 | 1418 |
| LLPAADMDDFSRQLQ | 0.1 | 0.2 | 1419 |
| LPAADMDDFSRQLQN | 0.1 | 0.2 | 1420 |
| PAADMDDFSRQLQNS | 0.1 | 0.2 | 1421 |
| AADMDDFSRQLQNSM | 0.2 | 0.2 | 1422 |
| ADMDDFSRQLQNSMS | 0.1 | 0.1 | 1423 |
| DMDDFSRQLQNSMSG | 0.1 | 0.2 | 1424 |
| MDDFSRQLQNSMSGA | 0.2 | 0.2 | 1425 |
| DDFSRQLQNSMSGAS | 0.2 | 0.2 | 1426 |
| DFSRQLQNSMSGASA | 0.1 | 0.2 | 1427 |
| FSRQLQNSMSGASAD | 0.1 | 0.2 | 1428 |
| SRQLQNSMSGASADS | 0.1 | 0.2 | 1429 |
| RQLQNSMSGASADST | 0.1 | 0.2 | 1430 |
| QLQNSMSGASADSTQ | 0.1 | 0.2 | 1431 |
| LQNSMSGASADSTQA | 0.2 | 0.2 | 1432 |

### REFERENCES

De Kruif J, Terstappen L, Boel E and Logtenberg T (1995a), Rapid selection of cell subpopulation-specific human monoclonal antibodies from a synthetic phage antibody library. Proc. Natl. Acad. Sci. USA 92:3938.

De Kruif J, Boel E and Logtenberg T (1995b), Selection and application of human single-chain Fv antibody fragments from a semi-synthetic phage antibody display library with designed CDR3 regions. J. Mol. Biol. 248:97-105.

Holmes K V. 2003. SARS coronavirus: a new challenge for prevention and therapy. J. Clin. Invest. 111, 1605-1609.

Ksiazek T G, et al. 2003. A novel coronavirus associated with severe acute respiratory syndrome. N. Eng. J. Med. 348, 1953-1966.

Marra M A, et al. 2003. The genome sequence of the SARS-associated coronavirus. Science 300, 1399-1404.

Rota P A, et al. 2003. Characterization of a novel coronavirus associated with severe acute respiratory syndrome. Science 300, 1394-1399.

Slootstra J W, et al. 1996. Structural aspects of antibody-antigen interaction revealed through small random peptide libraries. Mol. Divers. 1, 87-96.

1. An isolated peptide having an amino acid sequence selected from the group consisting of SEQ ID NO:9-SEQ ID NO:227, SEQ ID NO:229-SEQ ID NO:420, SEQ ID NO:492-SEQ ID NO:572, SEQ ID NO:592-SEQ ID NO:603, and SEQ ID NO:604.

2. The isolated peptide of claim 1, wherein said isolated peptide has an amino acid sequence selected from the group consisting of SEQ ID NO:358-SEQ ID NO:420, SEQ ID NO:545-SEQ ID NO:572, SEQ ID NO:592-SEQ ID NO:603, and SEQ ID NO:604.

3. The isolated peptide of claim 2, wherein said isolated peptide has an amino acid sequence selected from the group consisting of SEQ ID NO:358-SEQ ID NO:420, SEQ ID NO:545-SEQ ID NO:572, SEQ ID NO:592-SEQ ID NO:594, and SEQ ID NO:595.

4. The isolated peptide of claim 3, wherein said isolated peptide has an amino acid sequence selected from the group consisting of SEQ ID NO:360-SEQ ID NO:367 and SEQ ID NO:368.

5. A peptide comprising a part of the isolated peptide of claim 3, wherein said part comprises the amino acid sequence QGTTLPK (SEQ ID NO:606) and further wherein said part is recognized by antibodies present in serum derived from a subject that has been or is infected by Severe Acute Respiratory Syndrome Coronavirus (SARS-Co-V).

6. A second peptide consisting of an analogue of the isolated peptide of claim 3, wherein one or more amino acids of the isolated peptide of claim 3 are substituted, and wherein said analogue is recognized by antibodies present in serum derived from a subject that has been or is infected by Severe Acute Respiratory Syndrome Coronavirus (SARS-Co-V).

7. The isolated peptide of claim 2, wherein said isolated peptide has an amino acid sequence selected from the group consisting of SEQ ID NO: 592-SEQ ID NO:603 and SEQ ID NO:604.

8. The isolated peptide of claim 7, wherein said isolated peptide has an amino acid sequence selected from the group consisting of SEQ ID NO: 593-SEQ ID NO:598 and SEQ ID NO:599.

9. A second peptide comprising a part of the isolated peptide of claim 7, wherein said part is recognized by an antibody comprising a heavy chain CDR3 region having the amino acid sequence FNPFTSFDY (SEQ ID NO:587).

10. The second peptide of claim 9, wherein said part comprises an amino acid sequence RSAPRITFG (SEQ ID NO:605).

11. A second peptide consisting of an analogue of the isolated peptide of claim 7, wherein one or more amino acids of the isolated peptide of claim 7 are substituted in the isolated peptide of claim 7, and wherein said analogue is recognized by an antibody comprising a heavy chain CDR3 region having the amino acid sequence FNPFTSFDY (SEQ ID NO:587).

12. A fusion protein or a conjugate, wherein said fusion protein or conjugate comprises the peptide of claim 1.

13. An isolated nucleic acid molecule, wherein said isolated nucleic acid molecule encodes the isolated peptide of claim 1.

14. An isolated antibody, wherein said isolated antibody is able to specifically recognize the isolated peptide of claim 1.

15. The isolated antibody of claim 14, wherein said isolated antibody is a monoclonal antibody.

16. The isolated monoclonal antibody of claim 15, wherein said isolated monoclonal antibody is a human monoclonal antibody.

17. The isolated antibody of claim 14, wherein the isolated antibody has Severe Acute Respiratory Syndrome Coronavirus (SARS-Co-V) neutralizing activity.

18. An isolated nucleic acid molecule encoding the isolated antibody of claim 16.

19. A vector comprising at least one isolated nucleic acid molecule of claim 13.

20. A host comprising at least one vector of claim 19.

21. The host of claim 20, wherein the host is a cell.

22. A medicament or immunogen, wherein said medicament or immunogen comprises the isolated peptide of claim 1.

23. A vaccine comprising the isolated peptide of claim 22.

24. A medicament comprising the isolated antibody of claim 14.

25. A method for the detection, prevention and/or treatment of a condition in a subject resulting from a Severe Acute Respiratory Syndrome Coronavirus (SARS-Co-V), said method comprising the step of administering a medicament comprising the isolated peptide of claim 1 to the subject.

26. A method for the detection, prevention and/or treatment of a condition in a subject resulting from a Severe Acute Respiratory Syndrome Coronavirus (SARS-Co-V), said method comprising the step of administering a medicament comprising the isolated antibody of claim 14 to the subject.

27. A diagnostic test method for determining the presence of an antibody recognizing Severe Acute Respiratory Syndrome Coronavirus (SARS-Co-V) in a sample, said method comprising the steps of:
contacting said sample with the isolated peptide of claim 1 and
determining whether the antibody in the sample binds to the isolated peptide.

28. A diagnostic test method for determining the presence of Severe Acute Respiratory Syndrome Coronavirus (SARS-Co-V) in a sample, said method comprising the steps of:
contacting said sample with the isolated antibody of claim 14 and
determining whether the antibody in the sample binds to a molecule contained within said sample.

29. The diagnostic test method of claim 28, wherein the sample is from a human subject potentially infected with a Severe Acute Respiratory Syndrome Coronavirus (SARS-Co-V).

30. An isolated nucleic acid molecule encoding the fusion protein or conjugate of claim 12.

31. An isolated antibody able to specifically recognize the fusion protein or conjugate of claim 12.

32. A medicament comprising the fusion protein or conjugate of claim 12.

33. A medicament comprising the isolated nucleic acid molecule of claim 13.

34. A medicament comprising the isolated nucleic acid molecule of claim 18.

35. A method for the detection, prevention and/or treatment of a condition in a subject resulting from a Severe Acute Respiratory Syndrome Coronavirus (SARS-Co-V), said method comprising the step of administering a medicament comprising the fusion protein or conjugate of claim 12 to the subject.

36. A method for the detection, prevention and/or treatment of a condition in a subject resulting from a Severe Acute Respiratory Syndrome Coronavirus (SARS-Co-V), said method comprising the step of administering a medicament comprising the isolated nucleic acid molecule of claim 13 to the subject.

37. A method for the detection, prevention and/or treatment of a condition in a subject resulting from a Severe Acute Respiratory Syndrome Coronavirus (SARS-Co-V), said method comprising the step of administering a medicament comprising the isolated nucleic acid molecule of claim 18 to the subject.

38. A method for the detection, prevention and/or treatment of a condition in a subject resulting from a Severe Acute Respiratory Syndrome Coronavirus (SARS-Co-V), said method comprising the step of administering a medicament comprising the vector of claim 19 to the subject.

39. A diagnostic test method for determining the presence of an antibody recognizing Severe Acute Respiratory Syndrome Coronavirus (SARS-Co-V) in a sample, said method comprising the steps of:
contacting said sample with a peptide according to the fusion protein or conjugate of claim 12 and
determining whether the isolated antibody in the sample binds to the fusion protein or conjugate of claim 12.
